(19) United States
(12) Patent Application Publication
(10) Pub. No.: US 2019/0336615 A1
(43) Pub. Date: Nov. 7, 2019

(54) TUMOR TARGETING CONJUGATES AND METHODS OF USE THEREOF

(71) Applicant: SILVERBACK THERAPEUTICS, INC., Seattle, WA (US)

(72) Inventors: Peter Armstrong Thompson, Seattle, WA (US); Philip Huat Seng Tan, Seattle, WA (US); Peter Robert Baum, Seattle, WA (US); Robert Finley Dubose, Seattle, WA (US); Craig Alan Coburn, Seattle, WA (US); Sean Wesley Smith, Seattle, WA (US)

(21) Appl. No.: 16/476,640

(22) PCT Filed: Jan. 26, 2018
(86) PCT No.: PCT/US18/15607

§ 371 Date: Jul. 9, 2019

Related U.S. Application Data
(60) Provisional application No. 62/451,624, filed on Jan. 27, 2017.
(60) Provisional application No. 62/481,867, filed on Apr. 5, 2017.
(60) Provisional application No. 62/573,626, filed on Oct. 17, 2017.

(51) Int. Cl. A61K 47/68; C07K 16/32; C07K 16/28; A61P 35/00; A61K 9/00; C07K 16/30

(52) U.S. Cl. CPC A61K 47/6879; C07K 16/32; C07K 16/2878; A61P 35/00; A61K 9/0019; A61K 47/6803; A61K 2039/505; C07K 16/3023; C07K 2317/31; C07K 2317/52; C07K 2317/92; C07K 2317/732; C07K 16/3007

(57) ABSTRACT

Various compositions are disclosed. The compositions of conjugates comprising immune-stimulatory compounds are also provided. Additionally provided are the methods of preparation and use of the conjugates. This includes methods for treating disorders, such as cancer.

### PRIORITY

This application claims the benefit of priority under 35 U.S.C. § 119(e) to U.S. Provisional Application Nos. 62/451,624, filed Jan. 27, 2017; 62/481,867, filed Apr. 5, 2017; and 62/573,626, filed Oct. 17, 2017, each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 26, 2018, is named 50358-714_601_SL.txt and is 4,346,727 bytes in size.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The immune response may include the recruitment of immune cells that target tumors expressing these antigen markers. Additionally, the immune cells may express genes whose products are important to proper immune function and may serve as markers for specific types of immune cells. The boosting of this specific immune response has the potential to be a powerful anti-cancer treatment that can be more effective than conventional methods of cancer treatment and can have fewer side effects.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; c) an Fc comprising domain; and d) an immune-stimulatory compound attached to the recombinant bispecific antibody by a linker; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

In some aspects, a recombinant bispecific antibody, comprising: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; and c) an Fc comprising domain; and d) an immune-stimulatory compound attached to the recombinant bispecific antibody by a linker; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen. In some embodiments, the immune cell activation is measured by a cytokine release assay. In some embodiments, the immune cell activation by the recombinant bispecific antibody when the recombinant bispecific antibody is bound to the tumor associated antigen and to the antigen on the antigen presenting cell is at least two times, five times, or ten times greater than immune activation by the recombinant bispecific antibody when the recombinant bispecific antibody is bound to the antigen on the antigen presenting cell but not to the tumor associated antigen as measured by the cytokine release assay. In some embodiments, the immune cell activation by the recombinant bispecific antibody in the presence of cells having cell surface tumor associated antigen and antigen presenting cells having cell surface antigen is at least two times, five times, or ten times greater than immune cell activation by the recombinant bispecific antibody in the absence of the cells having cell surface tumor associated antigen as measured by the cytokine release assay. In some embodiments, the immune cell activation comprises an increase in one or more of: a) a secretion of one or more cytokines as measured by the cytokine release assay, b) a secretion of one or more chemokines as measured by an ELISA immunoassay, c) an expression level of one or more cell surface proteins associated with immune stimulation as measured by FACS, and d) an activity of one or more immune cell functions. In some embodiments, the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay. In some embodiments, the recombinant bispecific antibody induces tumor-cell directed antibody-dependent cell-mediated cytotoxicity. In some embodiments, the Fc comprising domain has one or more amino acid substitutions that decrease the binding affinity to one or more Fey receptors as compared to a wild-type Fc comprising domain. In some embodiments, the effector antigen binding domain has an increased binding affinity to the antigen on the antigen presenting cell as compared to the binding affinity of the effector antigen binding domain of an antibody that lacks the target antigen binding domain. In some embodiments, a K_{d} of the binding affinity of the effector antigen binding domain of the recombinant bispecific antibody to the antigen on the antigen presenting cell is increased by two times, five times, ten times, fifty times, or one-hundred times compared to the binding affinity of the effector antigen binding domain of an antibody that lacks the target antigen binding domain. In some embodiments, a K_{d} for binding of the effector antigen binding domain to the antigen on the antigen presenting cell is less than 20 nM, less than 100 nM, or less than 500 nM. In some embodiments, the Fc comprising domain is linked to the target antigen binding domain and to the effector antigen binding domain. In some embodiments, the target antigen binding domain comprises an immunoglobulin heavy chain variable region or antigen binding fragment thereof and an immunoglobulin light chain variable region or antigen binding fragment thereof. In some embodiments, the target antigen binding domain comprises a single chain variable region fragment (scFv). In some embodiments, the tumor associated antigen is an antigen selected from the group consisting of CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HERS, EPCAM, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PD-L1, CD204, CD206, CD301, VTCN1, and VISTA. In some embodiments, the tumor associated antigen is Her2/neu or p185HER2. In some embodiments, the target antigen binding domain comprises the following CDRs: a) HCDR1 comprising an amino acid sequence of SEQ ID NO: 13; b) HCDR2 comprising an amino acid sequence of SEQ ID NO: 14; c) HCDR3 comprising an amino acid sequence of SEQ ID NO: 15; d) LCDR1 comprising an amino acid sequence of SEQ ID NO: 18; e) LCDR2 comprising an amino acid sequence of SEQ ID NO: 19; and f) LCDR3 comprising an amino acid sequence of SEQ ID NO: 20; and wherein the recombinant bispecific antibody specifically binds to Her2/neu or p185HER2. In some embodiments, the target antigen binding domain comprises: a) a VH sequence having at least 80% sequence identity to an amino acid sequence of SEQ ID NO: 12; and b) a VL sequence having at least 80% sequence identity to an amino acid sequence of SEQ ID NO: 17. In some embodiments, the target antigen binding domain comprises: a) a heavy chain sequence having at least 80% sequence identity to an amino acid sequence of SEQ ID NO: 11; and b) a light chain sequence having at least 80% sequence identity to an amino acid sequence of SEQ ID NO: 16. In some embodiments, the target antigen binding domain comprises at least 80% sequence identity to the amino acid sequence between amino acid 20 and amino acid 110 of SEQ ID NO: 12 and at least 80% sequence identity to the amino acid sequence between amino acid 20 and amino acid 105 of SEQ ID NO: 17; and wherein the recombinant bispecific antibody specifically binds to Her2/neu or p185HER2. In some embodiments, the effector antigen binding domain comprises an immunoglobulin heavy chain variable region or antigen binding fragment thereof and an immunoglobulin light chain variable region or antigen binding fragment thereof. In some embodiments, the effector antigen binding domain comprises a single chain variable region fragment (scFv). In some embodiments, the scFv comprises at least 80% sequence identity to an amino acid sequence of SEQ ID NO: 1312. In some embodiments, the antigen presenting cell is a dendritic cell. In some embodiments, the antigen on the antigen presenting cell is a costimulatory molecule. In some embodiments, the antigen on the antigen presenting cell is selected from the group consisting of CD40, OX40L, DEC-205, 4-1BBL, CD36, CD204, MARCO, DC-SIGN, CLEC9A, CLECSA, Dectin 2, CLEC10A, CD206, CD64, CD32A, CD1A, HVEM, CD32B, PD-L1, or BDCA-2. In some embodiments, the effector antigen binding domain is a CD40 agonist. In some embodiments, the effector antigen binding domain comprises the following CDRs: a) HCDR1 comprising an amino acid sequence of SEQ ID NO: 3; b) HCDR2 comprising an amino acid sequence of SEQ ID NO: 4; c) HCDR3 comprising an amino acid sequence of SEQ ID NO: 5; d) LCDR1 comprising an amino acid sequence of SEQ ID NO: 8; e) LCDR2 comprising an amino acid sequence of SEQ ID NO: 9; and f) LCDR3 comprising an amino acid sequence of SEQ ID NO: 10. In some embodiments, the effector antigen binding domain comprises: a) a V_{H} sequence having at least 80% sequence identity to an amino acid sequence of SEQ ID NO: 2; and b) a VL sequence having at least 80% sequence identity to an amino acid sequence of SEQ ID NO: 7. In some embodiments, the effector antigen binding domain comprises: a) a heavy chain sequence having at least 80% sequence identity to an amino acid sequence of SEQ ID NO: 1; and b) a light chain having at least 80% sequence identity to an amino acid sequence of SEQ ID NO: 6. In some embodiments, the antigen on the antigen presenting cell is TREM2 or TNFR2. In some embodiments, the Fc comprising domain is linked C-terminal to the target antigen binding domain and N-terminal to the effector antigen binding domain. In some embodiments, the Fc comprising domain comprises one or more amino acid substitutions that reduce the affinity of the Fc comprising domain to an Fc receptor compared to the affinity of a reference Fc comprising domain to the Fc receptor in the absence of the one or more amino acid substitutions. In some embodiments, reference Fc comprising domain is selected from the group consisting of an Fc comprising domain having the amino acid sequence of SEQ ID NO: 1314, SEQ ID NO: 1315, SEQ ID NO: 1316, and SEQ ID NO: 1317. In some embodiments, reference Fc comprising domain comprises an amino acid sequence selected from the group consisting of SEQ ID NO: 1319, SEQ ID NO: 1320, SEQ ID NO: 1321, and SEQ ID NO: 1322. In some embodiments, the Fc comprising domain comprises a human IgG₁ Fc Region. In some embodiments, the one or more amino acid substitutions comprise L234A, L235A, G237A, and K322A, according to the EU index of Kabat. In some embodiments, the one or more amino acid substitutions comprise E233P, L234V, L235A, AG236, A327G, A330S, and P331S, according to the EU index of Kabat. In some embodiments, the Fc comprising domain comprises a human IgG₂ Fc Region. In some embodiments, the one or more amino acid substitutions comprises K322A, according to the EU index of Kabat. In some embodiments, the Fc comprising domain comprises a human IgG₂ₐ Fc Region. In some embodiments, the one or more amino acid substitutions comprises L235E, E318A, K320A, K322A, according to the EU index of Kabat. In some embodiments, the Fc comprising domain is an Fc null. In some embodiments, the Fc comprising domain has the amino acid sequence of SEQ ID NO: 1313. In some embodiments, the Fc comprising domain comprises the amino acid sequence of SEQ ID NO: 1318. In some embodiments, the Fc comprising domain is linked C-terminal to the target antigen binding domain and has the amino acid sequence of SEQ ID NO: 1311. In some embodiments, the linker links the immune-stimulatory compound to the Fc comprising domain. In some embodiments, the recombinant bispecific antibody further comprises an immune stimulatory compound and a linker, wherein the linker links the immune-stimulatory compound to the Fc comprising domain. In some embodiments, the immune-stimulatory compound is a damage-associated molecular pattern molecule or a pathogen-associated molecular pattern molecule. In some embodiments, the immune-stimulatory compound is a Toll-like receptor agonist, STING agonist, or RIG-I agonist. In some embodiments, the immune-stimulatory compound is a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663. In some embodiments, the immune-stimulatory compound is an inhibitor of TGFB, Beta-Catenin, PI3K-beta, STAT3, IL-10, IDO, or TDO. In some embodiments, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843. In some embodiments, the immune-stimulatory compound does not reduce the affinity of the recombinant bispecific antibody for binding to the tumor associated antigen or to the antigen on the antigen presenting cell. In some embodiments, the recombinant bispecific antibody further comprises a chemotherapeutic compound and a linker, wherein the linker links the chemotherapeutic compound to the Fc comprising domain. In some embodiments, the chemotherapeutic compound comprises an alkylating agent, an anthracycline, a cytoskeletal disruptor, a histone deacetylase inhibitor, an inhibitor of, a kinase inhibitor, a nucleoside analog or precursor analog, a peptide antibiotic, a platinum-based compound, or a plant alkaloid.

In some aspects, method of making a recombinant bispecific antibody comprises: a) producing an antibody construct comprising: i) a target antigen binding domain, wherein the target antigen binding domain specifically binds to a tumor associated antigen; ii) an effector antigen binding domain, wherein the effector antigen binding domain specifically binds to an antigen on an antigen presenting cell and the antigen is a molecule on the antigen presenting cell, wherein the antigen is a molecule on the antigen presenting cell; iii) an Fc comprising domain; and b) linking an immune-stimulatory compound to the antibody construct, wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a pharmaceutical composition comprises any recombinant bispecific antibody as described herein and a pharmaceutically acceptable carrier.

In some aspects, method of treating a subject in need thereof, comprising administering to the subject a therapeutic dose of any recombinant bispecific antibody as described herein or the pharmaceutical composition of any recombinant bispecific antibody as described herein. In some embodiments, the subject has cancer. In some embodiments, the recombinant bispecific antibody or the pharmaceutical composition is administered intravenously, cutaneously, subcutaneously, or injected at a site of affliction. In some embodiments, the recombinant bispecific antibody induces greater immune activation against a cancer as measured by a decrease in cancer cell number or volume as compared to non-cancerous tissue. In some embodiments, the recombinant bispecific antibody is administered intravenously to the subject at a minimum anticipated biological effect level of the recombinant bispecific antibody, a biological effect of the recombinant bispecific antibody is greater when the recombinant bispecific antibody is bound to the tumor associated antigen and to the antigen on the antigen presenting cell as compared to the biological effect of the recombinant bispecific antibody when it is not bound to the tumor associated antigen but is bound to the antigen on the antigen presenting cell; and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation. In some embodiments, the recombinant bispecific antibody is administered intravenously to the subject at the minimum anticipated biological effect level of the recombinant bispecific antibody, it induces a greater biological effect at the site of the cancer than at a non-cancerous site and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation.

In some aspects, a conjugate comprises: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the conjugate when bound to the tumor antigen as measured by a cytokine release assay is greater than immune cell activation in the absence of binding to the tumor antigen.

In some aspects, a conjugate comprises: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein antigen presenting cells are conditionally activated when the conjugate is bound to the tumor antigen as measured by a cytokine release assay.

In some aspects, an antibody construct comprises: a) a first binding domain, wherein the first binding domain specifically binds to a tumor antigen; b) a second binding domain, wherein the second binding domain specifically binds to an antigen on an antigen presenting cell, wherein the antigen is a molecule on the antigen presenting cell; and c) an Fc domain; wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain.

In some aspects, an antibody construct for use in inducing immune cell activation comprising: a) a first binding domain, wherein the first binding domain specifically binds to a tumor antigen; b) a second binding domain, wherein the second binding domain specifically binds to an antigen on an antigen presenting cell, wherein the antigen is a molecule on the antigen presenting cell; and c) an Fc domain; wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the antibody construct upon binding to tumor antigen as measured by a cytokine release assay is greater than immune cell activation caused by the antibody construct in the absence of binding to tumor antigen.

In some aspects, a conjugate for use in inducing immune cell activation comprising: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the conjugate when bound to the tumor antigen as measured by a cytokine release assay is greater than immune cell activation in the absence of binding to the tumor antigen.

In some aspects, a conjugate for use in conditionally activating an antigen presenting cell comprising: a) an antibody construct comprising: i) first binding domain, wherein the first binding domain specifically binds to a tumor antigen; ii) a second binding domain, wherein the second binding domain specifically binds to an antigen on the antigen presenting cell, and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein antigen presenting cells are conditionally activated when the conjugate is bound to the tumor antigen as measured cytokine release assay. In some embodiments, a K_{d} for binding of the first binding domain to the tumor antigen in the presence of the immune-stimulatory compound is no greater than about two times, five times, ten times, or fifty times a K_{d} for binding of the first binding domain to the tumor antigen in an absence of the immune-stimulatory compound In some embodiments, a Kd for binding of the second binding domain to the antigen on the antigen presenting cell in the presence of the immune-stimulatory compound is no greater than about two times, five times, ten times, or fifty times a Kd for binding of the second binding domain to the antigen on the antigen presenting cell in an absence of the immune-stimulatory compound. In some embodiments, a Kd for binding of the first binding domain to the tumor antigen is no greater than about 100 nM. In some embodiments, a Kd for binding of the second binding domain to the antigen on an antigen presenting cell is no greater than about 100 nM. In some embodiments, an amino acid sequence of the tumor antigen has at least 80% sequence identity with the amino acid sequence of a tumor antigen selected from the group consisting of HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, Her-2/neu, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, ETV6-AML, NY-BR-1, RGS5, SART3, Carbonic anhydrase IX, PAX5, 0Y-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, PD-L1, VTCN1 (B7-H4), VISTA, or a fragment thereof, and a fragment thereof. In some embodiments, an amino acid sequence of the tumor antigen has at least 80% sequence identity with the amino acid sequence of a tumor antigen selected from TABLE 1. In some embodiments, an amino acid sequence of the tumor antigen has at least 80% sequence identity with the amino acid sequence of a tumor antigen selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, Clorf186, and LRRC15, but not HER2 when the second binding domain specifically binds to CD40. In some embodiments, an amino acid sequence of the antigen on the antigen presenting cell has at least 80% sequence identity with the amino acid sequence of an antigen selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, and CD47, but not CD40 when the first binding domain specifically binds to HER2. In some embodiments, an amino acid sequence of the antigen on the antigen presenting cell has at least 80% sequence identity with the amino acid sequence of an antigen selected from TABLE 2. In some embodiments, the second binding domain is a CD40 agonist. In some embodiments, the first binding domain comprises a single chain variable fragment (scFv). In some embodiments, the second binding domain is a single chain variable fragment (scFv). In some embodiments, the second binding domain comprises a single chain variable fragment from an anti-CD40 antibody, an anti-DEC-205 antibody, an anti-CD36 mannose scavenger receptor 1 antibody, an anti-DC-SIGN antibody, an anti-CLEC9A antibody, an anti-CLEC12A antibody, an anti-BDCA-2 antibody, an anti-OX40L antibody, an anti-41BBL antibody, an anti-CD204 antibody, an anti-MARCO antibody, an anti-CLEC5A antibody, an anti-Dectin 1 antibody, an anti-Dectin 2 antibody, an anti-CLEC10A antibody, an anti-CD206 antibody, an anti-CD64 antibody, an anti-CD32A antibody, an anti-CD16A antibody, an anti-HVEM antibody, an anti-PD-L1, or an anti-CD32B antibody. In some embodiments, the second binding domain is attached to the Fc domain or the light chain of the first binding domain: a) as an Fc domain-second binding domain fusion peptide; b) as a light chain-second binding domain fusion peptide; or c) by a conjugation via a first linker. In some embodiments, the Fc domain is attached to the first binding domain: a) as an Fc domain-first binding domain fusion peptide; or b) by conjugation via a second linker. In some embodiments, the Fc domain is attached to both the first binding domain and to the second binding domain as a first binding domain-Fc domain-second binding domain fusion peptide. In some embodiments, the first binding domain is attached to both the Fc domain and the second binding domain as a first binding domain-second binding domain-Fc domain fusion peptide. In some embodiments, the first binding domain and the Fc domain comprise an antibody and the second binding domain comprises a single chain variable fragment (scFv). In some embodiments, the first binding domain has a set of variable region CDR sequences that comprises a set of variable region CDR sequences set forth in TABLE 3 or TABLE 4. In some embodiments, the second binding domain comprises a variable domain comprising a set of CDR sequences set forth in TABLE 11 or TABLE 12. In some embodiments, the first binding domain comprises a variable region comprising VH and VL sequences at least 80% sequence identity to a pair of VH and VL sequences set forth in TABLE 5 or TABLE 6. In some embodiments, the second binding domain comprises a variable region having VH and VL sequences having at least 80% sequence identity to a VH or VL sequence set forth in TABLE 13 or TABLE 14. In some embodiments, the first binding domain comprises an amino acid sequence having at least 80% sequence identity to any sequence in TABLE 7 or TABLE 8. In some embodiments, the second binding domain comprises an amino acid sequence having at least 80% sequence identity to any sequence in TABLE 15 or TABLE 16. In some embodiments, the second binding domain-Fc domain-first binding domain fusion peptide as described herein comprises an amino acid sequence having at least 80% sequence identity to a sequence in TABLE 9, TABLE 10, or TABLE 17. In some embodiments, the second binding domain-first binding domain-Fc domain fusion peptide as described herein comprises an amino acid sequence having at least 80% sequence identity to a sequence in TABLE 18 or TABLE 19.

In some aspects, a conjugate comprises: a) an immune-stimulatory compound; b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, Clorf186, LRRC15, DEC-205, CD36 mannose scavenger receptor 1, CLEC9A, DC-SIGN, CLEC12A, BDCA-2, 41BBL, MARCO, CLECSA, Dectin 1, Dectin 2, CD64, CD32A, CD16A, HVEM, and CD32B, and a fragment thereof; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate comprises: a) an immune-stimulatory compound; b) an antibody construct comprising a first binding domain and an Fc domain, wherein: i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, Clorf186, LRRC15, DEC-205, CD36 mannose scavenger receptor 1, CLEC9A, DC-SIGN, CLEC12A, BDCA-2, OX40L, 41BBL, MARCO, CLECSA, Dectin 1, Dectin 2, CD64, CD32A, CD16A, HVEM, CD32B, and CD47, and a fragment thereof, ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate comprises: a) an immune-stimulatory compound; b) an antibody construct comprising a first binding domain and an Fc domain, wherein: i) the first binding domain comprises a variable region comprising a set of CDR sequences that comprises at least 80% sequence identity to a set of variable region CDR sequences set forth in TABLE 3 or TABLE 11; ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate for use in activating an immune cell comprises: a) an immune-stimulatory compound; b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, Clorf186, LRRC15, DEC-205, CD36 mannose scavenger receptor 1, CLEC9A, DC-SIGN, CLEC12A, BDCA-2, 41BBL, MARCO, CLEC5A, Dectin 1, Dectin 2, CD64, CD32A, CD16A, HVEM, and CD32B, and a fragment thereof; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprises: a) an immune-stimulatory compound; b) an antibody construct comprising a first binding domain and an Fc domain, wherein: i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, Clorf186, LRRC15, DEC-205, CD36 mannose scavenger receptor 1, CLEC9A, DC-SIGN, CLEC12A, BDCA-2, OX40L, 41BBL, MARCO, CLEC5A, Dectin 1, Dectin 2, CD64, CD32A, CD16A, HVEM, CD32B, and CD47, and a fragment thereof, ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprises: a) an immune-stimulatory compound; b) an antibody construct comprising a first binding domain and an Fc domain, wherein the first binding domain comprises a variable region comprising a set of CDR sequences that comprises at least 80% sequence identity to a set of variable region CDR sequences set forth in TABLE 3 or TABLE 11; c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprising: a) an immune-stimulatory compound; b) an antibody construct comprising a first binding domain and an Fc domain, wherein: i) the first binding domain comprises a variable region comprising a set of CDR sequences that comprises at least 80% sequence identity to a set of variable region CDR sequences set forth in TABLE 3 or TABLE 11; ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen. In some embodiments, the first binding domain comprises a variable region comprising V_{H} and V_{L} sequences at least 80% sequence identity to a pair of V_{H} and V_{L} sequences set forth in TABLE 5 or TABLE 13. In some embodiments, the first binding domain comprises an amino acid sequence having at least 80% sequence identity to any sequence in TABLE 7 or TABLE 15. In some embodiments, a Kd for binding of the Fc domain to the Fc receptor in the presence of the immune-stimulatory compound is no greater than about two times, five times, ten times, or fifty times a Kd for binding of the Fc domain to the Fc receptor in an absence of the immune-stimulatory compound. In some embodiments, the immune-stimulatory compound is a damage-associated molecular pattern molecule or pathogen-associated molecular pattern molecule. In some embodiments, the immune-stimulatory compound is a toll-like receptor agonist, STING agonist, or RIG-I agonist. In some embodiments, the immune-stimulatory compound is a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663. In some embodiments, the immune-stimulatory compound is an inhibitor of TGFB, Beta-Catenin, TNIK, Tankyrase, PI3K-beta, STAT3, IL-10, IDO, or TDO. In some embodiments, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843. In some embodiments, the Fc domain is an Fc domain variant comprising at least one amino acid residue change as compared to a wild type sequence of the Fc domain. In some embodiments, the Fc domain variant binds to an Fc receptor with altered affinity as compared to the wild type Fc domain. In some embodiments, the at least one amino acid residue change is selected from a group consisting of: a) F243L, R292P, Y300L, L235V, and P396L, wherein numbering of amino acid residues in the Fc domain is according to the EU index; b) S239D and I332E, wherein numbering of amino acid residues in the Fc domain is according to the EU index; and c) S298A, E333A, and K334A, wherein numbering of amino acid residues in the Fc domain is according to the EU index. In some embodiments, the antibody construct or conjugate induces secretion of cytokines by an immune cell as measured by a cytokine release assay. In some embodiments, the cytokine is IFN-γ, IL-8, IL-12, IL-2, or a combination thereof. In some embodiments, the antibody construct or conjugate induces antigen presentation on a dendritic cell, B cell, macrophage, or a combination thereof.

In some aspects, a method of making a conjugate comprises linking an antibody construct as described herein to an immune stimulatory compound by a linker.

In some aspects, a pharmaceutical composition comprises the conjugate or antibody construct of as described herein and a pharmaceutically acceptable carrier.

In some aspects, a method of treatment for a subject in need thereof comprises administering a therapeutic dose of the antibody construct or conjugate as described herein or the pharmaceutical composition as described herein. In some embodiments, the subject has cancer. In some embodiments, the antibody construct or conjugate is administered intravenously, cutaneously, subcutaneously, or injected at a site of affliction. In some embodiments, after administration of antibody construct or conjugate to the subject, immune cell activation is increased in the subject as measured by a secretion of one or more cytokines as measured by a cytokine release assay, a secretion of one or more chemokines as measured by an ELISA immunoassay, an expression level of one or more cell surface proteins associated with immune stimulation as measured by an ELISA immunoassay, an activity of one or more immune cell functions, or combination thereof, as compared to before administration of the antibody construct or conjugate to the subject. In some embodiments, the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay. In some embodiments, after administration of the antibody construct or conjugate to the subject, tumor cell intracellular signaling is altered in the subject as compared to tumor cell intracellular signaling before administration of the antibody construct or conjugate as measured by an intracellular signaling assay. In some embodiments, the altered tumor cell intracellular signaling increases tumor immunogenicity as measured by an immunogencity assay.

In some aspects, a kit comprising a pharmaceutically acceptable dosage unit of a pharmaceutically effective amount of the conjugate or antibody construct as described herein or the pharmaceutical composition as described herein.

Described herein are recombinant bispecific antibodies useful in the treatment of cancer. The recombinant antibodies according to the current disclosure are bispecific antibodies that can comprise at least two different antigen binding domains that are coupled to an Fc comprising domain. This recombinant antibody can exhibit more potent immune activation when both antigen binding domains are bound to their respective antigen. One example method for increasing immune activation when both antigen binding domains are bound to their respective antigen can be accomplished by a recombinant antibody coupled to an Fc comprising domain that exhibits reduced affinity to an Fc receptor. Another example method for achieving an increased immune activation when both antigen binding domains are bound to their respective antigen can be accomplished by using a binding domain with a low avidity for its antigen as one of the antigen binding domains in the recombinant antibody. One binding domain of the bispecific antibody can specifically bind to a tumor associated antigen and another binding domain can specifically bind to a molecule on the surface of an antigen presenting cell (APC), such as a macrophage or dendritic cell. Thus, the two binding domains cooperate to bring APCs to cancerous cells or tumors allowing the APC to initiate/propagate a cancer cell/tumor specific immune response through cytokine release, chemokine release, or presentation of tumor associated antigens to effector or helper T cells.

Therapy with recombinant monoclonal or bispecific antibodies can generally be well tolerated, however, antibodies directed to immune response stimulating receptors on immune cells can result in systemic toxic release of cytokines and other immune modulators that can limit their clinical use or dose, thereby limiting their effectiveness in generating patient anti-tumor responses. This immune activation can be especially non-beneficial when it occurs systemically in the absence of tumor antigens. The systemic agonism exhibited by antibodies to many APC receptors can depend upon high affinity binding to the APC antigen and higher order cross-linking of the APC receptors by clustering of the cell bound antibodies. Many studies show this can be mediated by Fc gamma Receptor (FcγR) binding to the Fc domain of the antibodies, and cross-linking of different antibody molecules and their bound APC immune stimulating receptors. An additional complication of cross-linking by FcγR can be antibody dependent cell mediated cytotoxicity (ADCC) of the APCs resulting in lowered immune response to tumors and pathogens. ADCC can be attributed to the antibody Fc region which binds to FcγRs on effector cells (e.g., NK cells). Two non-mutually exclusive solutions to the above can be contemplated. In one, elevating the threshold for FcγR binding can reduce excessive systemic immune activation and unwanted ADCC directed to APCs of antibody therapy. In the second, the affinity of the antibody for its APC target can be lowered so that effective agonistic binding of antibody molecules to APCs can be driven by avidity, preferentially found when the bispecific antibody is bound to its tumor antigen target. As described herein, the Fc comprising region of the recombinant bispecific antibody can contain one or more mutations that can reduce binding to an FcγR. Alternatively, the Fc region can be derived from an IgG subclass that can bind FcγRs with low affinity, for example IgG₂. Fc receptors can be highly expressed on different antigen presenting cells such as dendritic cells, and their engagement can lead to activation of the immunostimulatory and antigen presenting function of these cells. By reducing binding of the Fc region to the FcγR the threshold for APC activation can be raised. By raising the threshold for APC activation, the possibility of a damaging immune/inflammatory response to healthy, non-cancerous tissue can be reduced. Attenuating activation by modifications made to the Fc regions can result in superior bioavailability and lower side effects. Also described herein are bispecific antibodies with high affinity anti-tumor antigen binding and low affinity immune receptor binding such that APC activation can be increased when the bispecific antibody is bound to its tumor antigen. As a result, the antibodies of this disclosure generally can have a higher maximum tolerated dosage, and can be administered at levels higher than therapeutic antibodies not modified as described herein.

In some embodiments, the recombinant bispecific antibody further comprises a chemotherapeutic compound and a linker, wherein the linker links the chemotherapeutic compound to the Fc comprising domain. In some aspects, the chemotherapeutic compound comprises an alkylating agent, an anthracycline, a cytoskeletal disruptor, a histone deacetylase inhibitor, an inhibitor of, a kinase inhibitor, a nucleoside analog or precursor analog, a peptide antibiotic, a platinum-based compound, or a plant alkaloid.

In some aspects, the recombinant bispecific antibody specifically binds to the tumor associated antigen in a cluster of recombinant antibodies and induces a signal in the antigen presenting cell. In some aspects, the recombinant antibody specifically binds to the tumor associated antigen in a cluster of recombinant antibodies and results in an increased avidity for the molecule on the antigen presenting cell. In some aspects, a recombinant antibody density resulting from the recombinant antibody binding to the tumor associated antigen induces signaling in the antigen presenting cell. In some aspects, the recombinant antibody density of greater than 5000 antibodies per cell resulting from the recombinant antibody specifically binding to the tumor associated antigen induces signaling in the antigen presenting cell.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 illustrates a schematic of an antibody construct comprising an antibody and a second binding domain. An antibody can comprise two heavy chains as shown in gray and two light chains as shown in light gray. A portion of the heavy chains can comprise Fc domains (705 and 720). An antibody can comprise a binding domain comprising two antigen binding sites (710 and 715). The second binding domain can be attached to the antibody (780 and 785), for example, at the C-terminus of the heavy chains.

FIG. 2 illustrates a schematic of an exemplary conjugate. A conjugate can comprise an antibody, which can comprise two heavy chains as shown in gray and two light chains as shown in light gray. The antibody can comprise a binding domain comprising two antigen binding sites (910 and 915), and a portion of the heavy chains contain Fc domains (905 and 920). The immune-stimulatory compounds (930 and 940) can be conjugated to the antibody by linkers (960 and 970). A second binding domain can be attached to the antibody (980 and 985), for example, at the C-terminus of the heavy chains.

FIG. 3 illustrates a schematic of an exemplary conjugate. A conjugate can comprise the Fc region of an antibody with the heavy chains shown in gray, and two scaffolds as shown in light gray. The conjugate can comprise a first binding domain comprising two antigen binding sites (1110 and 1115) in the scaffolds, and a portion of the heavy chains can comprise Fc domains (1105 and 1120). The immune-stimulatory compounds (1130 and 1140) can be conjugated to the scaffolds by linkers (1160 and 1170). A second binding domain can be attached to the conjugate (1180 and 1185), for example, at the C-terminus of the heavy chains.

FIG. 4 illustrates a schematic of an exemplary conjugate. A conjugate can comprise the F(ab′)2 region of an antibody with heavy chains shown in gray and light chains shown in light gray, and two scaffolds as shown in dark gray. The conjugate can comprise a first binding domain comprising two antigen binding sites (1310 and 1315), and a portion of two scaffolds contain Fc domains (1340 and 1345). The immune-stimulatory compounds (1330 and 1340) can be conjugated to the scaffold by linkers (1360 and 1370). A second binding domain can be attached to the conjugate (1380 and 1385).

FIG. 5 illustrates a schematic of an exemplary conjugate. A conjugate can comprise two scaffolds as shown in light gray and two scaffolds as shown in dark gray. The conjugate can comprise a first binding domain comprising two antigen binding sites (1510 and 1515), and a portion of the two dark gray scaffolds contain Fc domains (1540 and 1545). The immune-stimulatory compounds (1530 and 1535) can be conjugated to the scaffolds by linkers (1560 and 1570). A second binding domain can be attached to the conjugate (1580 and 1585).

FIG. 6 illustrates a CLUSTAL O(1.2.1) multiple amino acid sequence alignment of the amino acid sequences of SBT-040-G1VLPLL (SEQ ID NO: 1323), SBT-040-G1AAA (SEQ ID NO: 1324), SBT-040-G1WT (SEQ ID NO: 1325), and SBT-040-G1DE (SEQ ID NO: 1326). The SBT-040-G1VLPLL sequence is an amino acid sequence of an IgG1 isotype heavy chain of a human CD40 monoclonal antibody SBT-040 containing L235V, F243L, R292P, Y300L, and P396L amino acid residue modifications of a wild type IgG1 Fc domain. The L235V, F243L, R292P, Y300L, and P396L amino acid residue modifications are in bold. The SBT-040-G1AAA sequence is an amino acid sequence of an IgG1 isotype heavy chain of a human CD40 monoclonal antibody SBT-040 containing S298A, E333A, and K334A amino acid residue modifications of a wild type IgG1 Fc domain. The S298A, E333A, and K334A amino acid residue modifications are italics. The SBT-040-G1WT sequence is an amino acid sequence of an IgG1 isotype heavy chain of a human CD40 monoclonal antibody SBT-040. The SBT-040-G1AAA sequence is an amino acid sequence of an IgG1 isotype heavy chain of a human CD40 monoclonal antibody SBT-040 containing S239D and I332E amino acid residue modifications bold italics. Additionally, the hinge region of each amino acid sequence is differentiated from other regions of the amino acid sequence by brackets. The left bracket indicates the upper portion of the hinge region (UH). The four residues between the brackets are the middle portion of the hinge region. The right bracket indicates the lower portion of the hinge region (LH). SEQ ID NO: 1327 is the sequence of SBT-040-G1VLPLL without the leader sequence. SEQ ID NO: 1328 is the sequence of SBT-040-G1AAA without the leader sequence. SEQ ID NO: 577 is the sequence of SBT-040-G1WT without the leader sequence. SEQ ID NO: 1329 is the sequence of SBT-040-G1DE without the leader sequence.

FIGS. 7A and 7B illustrate that a bispecific anti-HER2×anti-CD40 IgG1 conjugate (HER2-CD40G1) and a bispecific anti-HER2×anti-CD40 IgG1 Fc null antibody (HER2-CD40 G1null) had decreased binding to CD40 on monocyte-derived dendritic cells (moDCs) compared to the parental anti-CD40 monoclonal antibody (SBT-040G1). moDCs were stained with either SBT-040G1, HER2-CD40G1 and HER2-CD40 G1ₙᵤₗₗ at equivalent molar concentrations. A secondary goat anti-human IgG polyclonal antibody was used to detect SBT-040G1, HER2-CD40G1 or HER2-CD40ₙᵤₗₗ binding by flow cytometry. MFI fold change was calculated as (MFI test Ab/MFI isotype control).

FIG. 8A illustrates activation of dendritic cells (DCs) was dependent on CD40 agonism and Fc receptor agonism by bispecific anti-HER2-anti-CD40 IgG1 antibody construct (HER2-CD40G1) bound to the tumor antigen HER2 as shown by increased expression of CD86. This figure also illustrates the anti-HER2×anti-CD40 IgG1 Fc null antibody (HER2-CD40 G1ₙᵤₗₗ) conditional activation of dendritic cells (DCs) when bound to the tumor antigen HER2. CD86 was measured by flow cytometry on DCs co-cultured with CHO cells with or without HER2 expression in the presence of the HER2-CD40G1 antibody construct, anti-HER2-anti-CD40 IgG1 Fc null (HER2-CD40G1ₙᵤₗₗ), or the parental anti-CD40 monoclonal antibody (SBT-040G1) at the indicated concentrations. Each data point was generated from pooled duplicate samples. HER2⁺ CHO indicates co-culture with HER2 expressing CHO cells; HERT CHO indicates a co-culture with CHO cells that were not expressing HER2.

FIG. 8B illustrates activation of dendritic cells (DCs) was dependent on CD40 agonism and Fc receptor agonism by bispecific anti-HER2-anti-CD40 IgG1 antibody construct (HER2-CD40G1) bound to the tumor antigen HER2 as shown by increased expression of CD83. This figure also illustrates anti-HER2×anti-CD40 IgG1 Fcₙᵤₗₗ (HER2-CD40G1ₙᵤₗₗ) antibody conditional activation of dendritic cells (DCs) when bound to the tumor antigen HER2. CD83 was measured by flow cytometry on DCs co-cultured with CHO cells with or without HER2 expression in the presence of the HER2-CD40G1 antibody construct, anti-HER2×anti-CD40 IgG1 Fcₙᵤₗₗ (HER2-CD40G1ₙᵤₗₗ), or the parental anti-CD40 monoclonal antibody (SBT-040G1) at the indicated concentrations. Each data point was generated from pooled duplicate samples. HER2⁺ CHO indicates co-culture with HER2 expressing CHO cells; HERT CHO indicates a co-culture with CHO cells that were not expressing HER2.

FIG. 9 illustrates macrophage-mediated antibody-dependent cellular cytotoxicity (ADCC) of HER2⁺ target cells was efficiently induced by bispecific anti-HER2×anti-CD40 IgG1 antibody construct (HER2-CD40G1). Monocyte-derived macrophages were generated by culturing monocytes for 7 days in the presence of GM-CSF. Macrophages were plated with HER2-expressing CHO cells at a 2:1 ratio in the presence of titrating concentrations of HER2-CD40G1 antibody construct, anti-HER2-anti-CD40 IgG1 Fc null antibody construct (HER2-CD40G1ₙᵤₗₗ), parental anti-CD40 monoclonal antibody (SBT-040G1), or parental anti-HER2 monoclonal antibody (SBT-050G1). After 24 hours, CHO viability was assessed by flow cytometry as a readout of ADCC activity.

FIG. 10 illustrates schematics for three separate non-limiting embodiments of recombinant bispecific antibodies.

FIG. 11 illustrates a schematic of an antibody construct comprising an antibody and a second binding domain. An antibody can comprise two heavy chains as shown in gray and two light chains as shown in light gray. A portion of the heavy chains can comprise Fc domains (1705 and 1720). An antibody can comprise a binding domain comprising two antigen binding sites (1710 and 1715). The second binding domain can be attached to the antibody (1780 and 1785), for example, at the C-terminus of the light chains.

FIG. 12 illustrates a schematic of an exemplary conjugate. A conjugate can comprise an antibody, which contains two heavy chains as shown in gray and two light chains as shown in light gray. The antibody can comprise a binding domain comprising two antigen binding sites (1910 and 1915), and a portion of the heavy chains can comprise Fc domains (1905 and 1920). The immune-stimulatory compounds (1930 and 1940) can be conjugated to the antibody by linkers (1960 and 1970). A second binding domain can be attached to the antibody (1980 and 1985), for example, at the C-terminus of the light chains.

FIG. 13 illustrates a schematic of an exemplary conjugate. A conjugate can comprise the Fc region of an antibody shown in gray, and two scaffolds as shown in light gray. The conjugate can comprise a first binding domain comprising two antigen binding sites (2110 and 2115) in the scaffolds, and a portion containing Fc domains (2105 and 2120). The immune-stimulatory compounds (2130 and 2140) can be conjugated to the scaffolds by linkers (2160 and 2170). A second binding domain can be attached to the conjugate (2180 and 2185).

FIG. 14 illustrates a schematic of an exemplary conjugate. A conjugate can comprise the F(ab′)2 region of an antibody with heavy chains shown in gray and light chains shown in light gray, and two scaffolds as shown in dark gray. The conjugate can comprise a first binding domain comprising two antigen binding sites (2310 and 2315), and a portion of two scaffolds can comprise Fc domains (2340 and 2345). The immune-stimulatory compounds (2330 and 2340) can be attached to the scaffolds by linkers (2360 and 2370). A second binding domain can be attached to the conjugate (2380 and 2385), for example, at the C-terminus of the light chains.

FIG. 15 illustrates a schematic of an exemplary conjugate. A conjugate can comprise two scaffolds as shown in light gray and two scaffolds as shown in dark gray. The conjugate can comprise a first binding domain comprising two antigen binding sites (2510 and 2515), and a portion of the two dark gray scaffolds contain Fc domains (2540 and 2545). The immune-stimulatory compounds (2530 and 2540) can be attached to the scaffolds by linkers (2560 and 2570). A second binding domain can be attached to the conjugate (2580 and 2585).

FIG. 16 illustrates a schematic of an antibody construct comprising an antibody. An antibody can comprise two heavy chains and two light chains. A portion of the heavy chains can comprise Fc domains (2705 and 2720). An antibody can comprise a binding domain comprising two antigen binding sites shown in black (2710 and 2715).

FIG. 17 illustrates a schematic of an antibody construct comprising an antibody. An antibody can comprise two heavy chains and two light chains. A portion of the heavy chains can comprise Fc domains (2925 and 2930). An antibody can comprise a first binding domain comprising two antigen binding sites shown in black (2910 and 2915). An antibody can comprise a second binding domain comprising two single chain variable fragments (2905 and 2920) attached to a C-terminus of the light chains. A single chain variable fragment can be attached to a light chain chain at a heavy chain variable domain of the single chain variable fragment. A single chain variable fragment can be attached to a light chain at a light chain variable domain of the single chain variable fragment.

FIG. 18 illustrates a schematic of an antibody construct comprising an antibody. An antibody can comprise two heavy chains and two light chains. A portion of the heavy chains can comprise Fc domains (3120 and 3125). An antibody can comprise a first binding domain comprising two antigen binding sites shown in black (3110 and 3115). An antibody can comprise a second binding domain comprising two single chain variable fragments (3130 and 3135) attached to a C-terminus of the heavy chains. A single chain variable fragment can be attached to a heavy chain chain at a heavy chain variable domain of the single chain variable fragment. A single chain variable fragment can be attached to a heavy chain at a light chain variable domain of the single chain variable fragment.

FIG. 19 illustrates a schematic of an antibody construct comprising an antibody. An antibody can comprise two heavy chains and two light chains. A portion of the heavy chains can comprise Fc domains (3330 and 3335). An antibody can comprise a first binding domain comprising two antigen binding sites shown in black (3310 and 3315). An antibody can comprise a second binding domain comprising two single chain variable fragments (3320 and 3325) attached to a C-terminus of the light chains. A single chain variable fragment can be attached to a light chain chain at a heavy chain variable domain of the single chain variable fragment. A single chain variable fragment can be attached to a light chain at a light chain variable domain of the single chain variable fragment. An antibody can comprise a third binding domain comprising two single chain variable fragments (3340 and 3345) attached to a C-terminus of the heavy chains. A single chain variable fragment can be attached to a heavy chain chain at a heavy chain variable domain of the single chain variable fragment. A single chain variable fragment can be attached to a heavy chain at a light chain variable domain of the single chain variable fragment.

FIG. 20 shows that HER2-TLR8 agonist conjugates and HER2×CD40 TLR8 agonist conjugates were active in the presence of PBMCs and SKBR3 cells that express HER2, as measured by TNFα production. HER2 antibody is HER2-G1WT.

FIG. 21 shows that TROP2(TROP2-G1WT)-TLR8 agonist conjugates were active in the presence of PBMCs and SKBR3 cells that express HER2, as measured by TNFα production. TROP2 antibody is TROP2-G1WT.

FIG. 22 shows that a CEA-TLR8 agonist conjugate was active in the presence of monocytes and CHO cells engineered to express CEA, while the CEA antibody alone, and the control antibodies (HER2-G1WT) and conjugates were not active, as measured by TNFα production. CEA antibody is CEA-G1WT.

FIG. 23 shows that an anti-CEA-TLR8 agonist conjugate and a CEA×CD40 TLR8 agonist conjugate were active in the presence of monocytes and SKCO-1 cells, as measured by TNFα production. CEA antibody is CEA-G1WT and bispecific CEA×CD40 antibody is CEA×CD40-G1WT.

FIG. 24 shows that a TROP TRL8 agonist conjugate was active in a dose-dependent manner on various cell lines expressing TROP2.

FIG. 25 shows that a TROP2 TLR8 agonist conjugate was active in a dose-dependent manner on various cell lines expressing TROP2.

FIG. 26 shows that a HER2×CD40 bispecific antibody conjugate was able to activate monocyte-derived dendritic cells.

FIG. 27 shows that a HER2×CD40 bispecific antibody conjugate was further able to stimulate T cells in the presence of HER2 positive tumor cells.

FIGS. 28A, 28B, and 28C show that activation of primary B cells (CD86 expression) was increased by bispecific HER2×CD40 recombinant antibody conjugate as compared to a Her2 recombinant antibody conjugate.

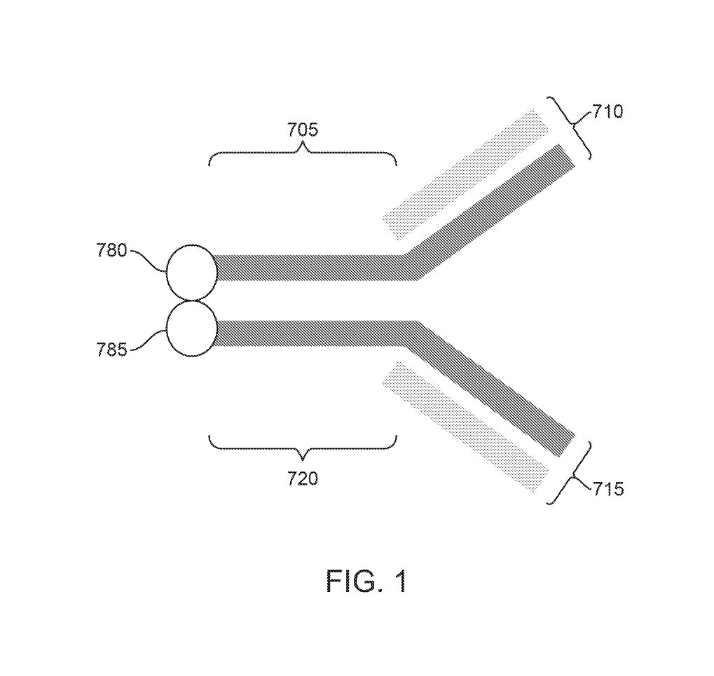

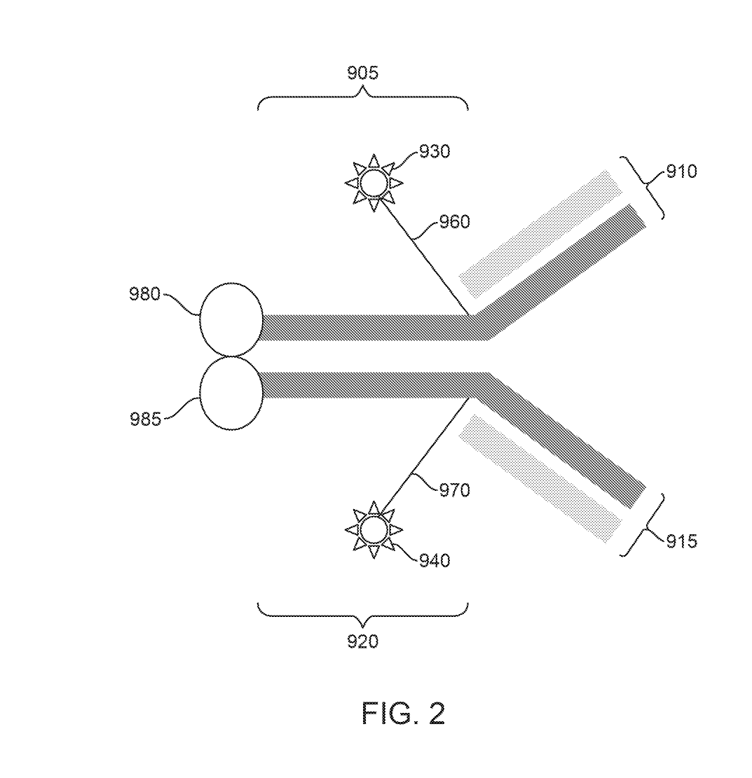

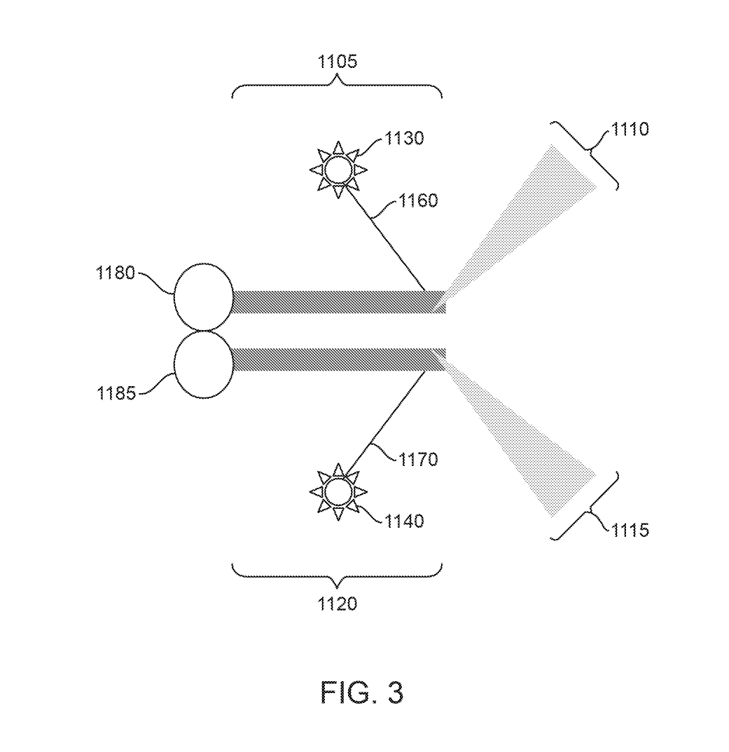

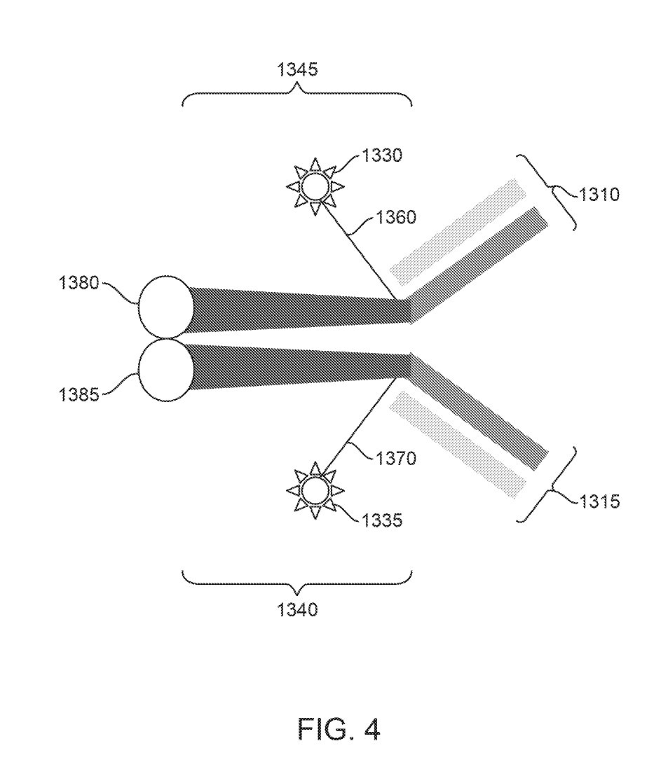

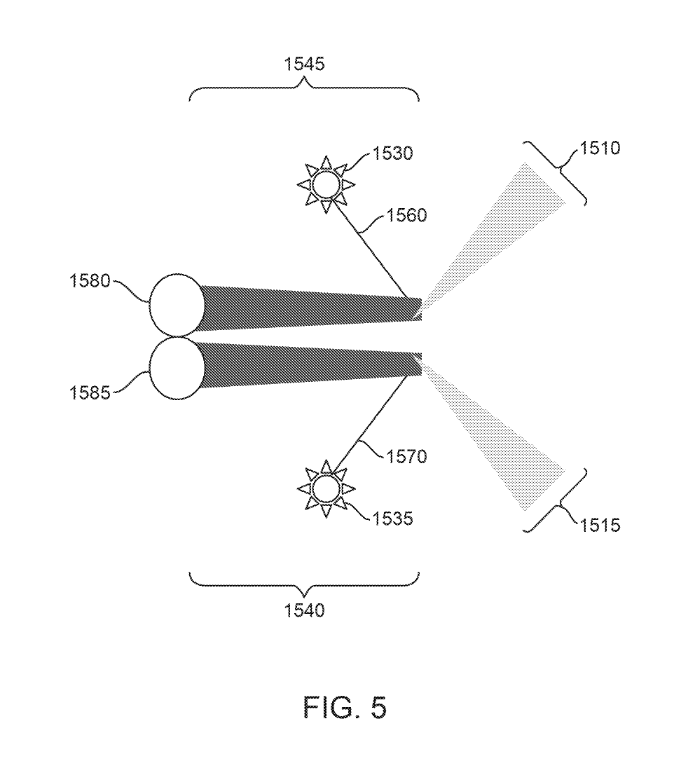

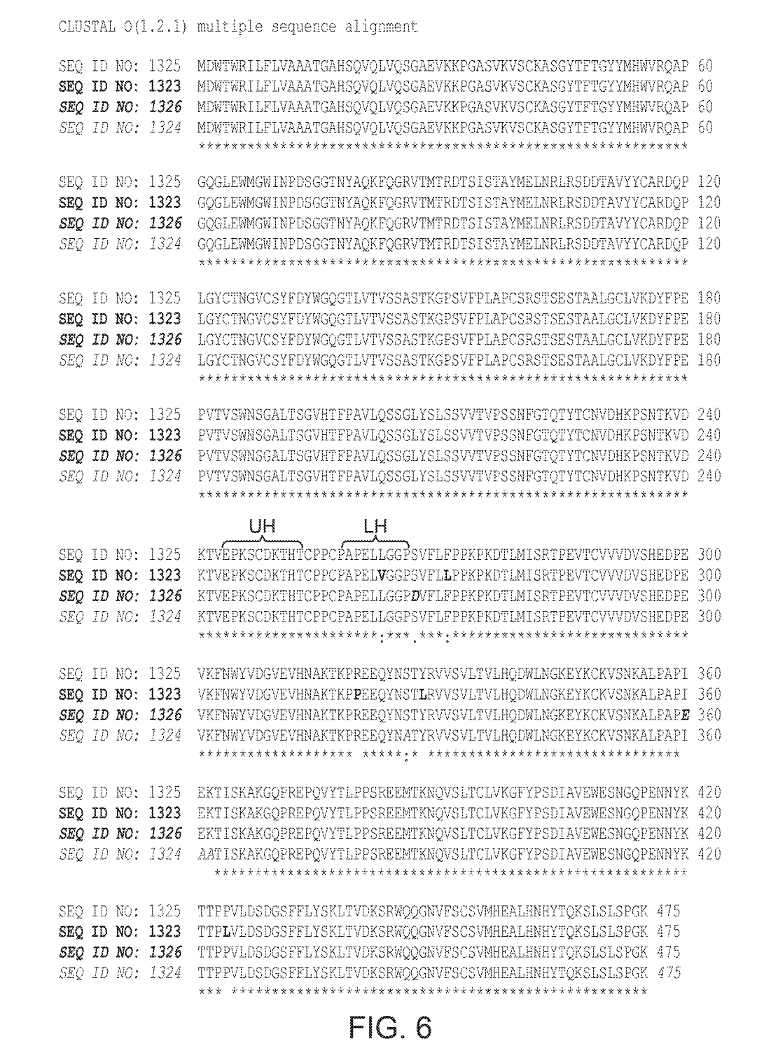

### DETAILED DESCRIPTION

Additional aspects and advantages of the present disclosure will become apparent to those skilled in this art from the following detailed description, wherein illustrative aspects of the present disclosure are shown and described. As will be realized, the present disclosure is capable of other and different aspects, and its several details are capable of modifications in various respects, all without departing from the disclosure. Accordingly, the drawings and description are to be regarded as illustrative in nature, and not as restrictive.

As used herein, “homologous” or “homology” refers to the similarity between a DNA, RNA, nucleotide, amino acid, or protein sequence to another DNA, RNA, nucleotide, amino acid, or protein sequence. Homology can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, Fab′, F(ab′)₂, Fab, Fv, rIgG, scFv, hcAbs (heavy chain antibodies), a single domain antibody, V_{HH}, V_{NAR}, sdAbs, or nanobody.

As used herein a “recombinant antibody” is an antibody that comprises an amino acid sequence derived from two different species or, or two different sources, and includes synthetic molecules. By way of non-limiting example, an antibody that comprises a non-human CDR and a human variable region framework or constant or Fc region, an antibody with binding domains from two different monoclonal antibodies, or an antibody comprising a mutation of one or more amino acid residues to increase or decrease biological activity or binding of a part of the antibody. In certain embodiments, recombinant antibodies are produced from a recombinant DNA molecule or synthesized. In certain embodiments, the antibodies described herein are a polypeptide(s) encoded by one or more polynucleotides.

As used herein, “recognize” refers to the association or binding between an antigen binding domain and an antigen.

As used herein, an “antigen” refers to an antigenic substance that can trigger an immune response in a host. An antigenic substance can be a molecule, such as a costimulatory molecule (e.g., CD40, OX40L, 4-1BBL, DEC-205, etc.) that can trigger an immune response in a host.

As used herein, a “tumor antigen” refers to an antigenic substance associated with a tumor or cancer cell, and that can trigger an immune response in a host.

As used herein, an “antigen on an antigen presenting cell” refers to an antigenic substance associated with an antigen presenting, and that can trigger an immune response in a host.

As used herein, an “antibody construct” refers to a construct that contains an antigen binding domain and an Fc domain.

As used herein, a binding domain refers to an antibody or non-antibody domain.

As used herein, an “antigen binding domain” refers to a binding domain from an antibody or from a non-antibody that can bind to an antigen. An antigen binding domain can be a tumor antigen binding domain or a binding domain that can bind to an antigen (such as a molecule) on an antigen presenting cell. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen or different antigens (e.g., first antigen binding domain that can bind to a tumor antigen, second antigen binding domain that can bind to a molecule on an antigen presenting cell (APC antigen), and third antigen binding domain that can bind to an APC antigen).

As used herein, an “antibody antigen binding domain” refers to a binding domain from an antibody that can bind to an antigen.

As used herein, an “Fc domain” refers to an Fc domain from an antibody or from a non-antibody that can bind to an Fc receptor. As used herein, an “Fc domain” and an “Fc comprising domain” can be used interchangeably.

As used herein, a “target binding domain” refers to a construct that contains an antigen binding domain from an antibody or from a non-antibody that can bind to an antigen.

As used herein, an “ATAC” refers to a construct of an immune-stimulatory compound and a linker.

As used herein, a “conjugate” refers to an antibody construct attached to an immune-stimulatory molecule.

As used herein, a “bispecific tumor targeting antibody construct” refers to a structure that comprises a tumor antigen binding domain, a binding domain that binds to a molecule on an immune cell, such as an antigen presenting cell, and an Fc domain. A bispecific tumor targeting conjugate refers to bispecific tumor targeting antibody construct attached to an immune-stimulatory compound. As used herein, a “bispecific tumor targeting antibody construct” is used interchangeably with a “recombinant bispecific antibody”. As used herein, a “bispecific tumor targeting antibody conjugate” is used interchangeably with a “recombinant bispecific antibody conjugate”.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, “minimum anticipated biological effect level” (MABEL) is the anticipated dose needed that results in a biological effect in a human subject, in which a biological effect is measured by an in vitro, ex vivo, and/or in vivo assay that measures a selected biological, biochemical, pharmacological, or pharmacodynamic effect. A selected biological, biochemical, pharmacological, or pharmacodynamic effect can be secretion of one or more cytokines, secretion of one or more chemokines, expression level of one or more cell surface proteins associated with immune stimulation, or activity of one or more immune cell functions. Cytokine release can be measured by a cytokine release assay. Chemokine secretion can be measured by an ELISA immunoassay. Expression level of one or more cell surface proteins associated with immune stimulation can be measured by Fluorescent-Activated Cell Sorting (FACS). Activity of one or more immune cell functions can be antibody-dependent cell-mediated cytotoxicity (ADCC), antibody dependent cellular phagocytosis (ADCP), or antigen cross-presentation. ADCC can be measured by an ADCC assay. ADCP can be measured by an ACDP assay. Antigen cross-presentation can be measured by a cross-presentation assay.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings wherein at least one of the rings includes a heteroatom. In an exemplary embodiment, an aromatic ring, e.g., pyridyl, may be fused to a saturated or unsaturated ring, e.g., cyclohexane, cyclopentane, morpholine, piperidine or cyclohexene. The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously, undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure. the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

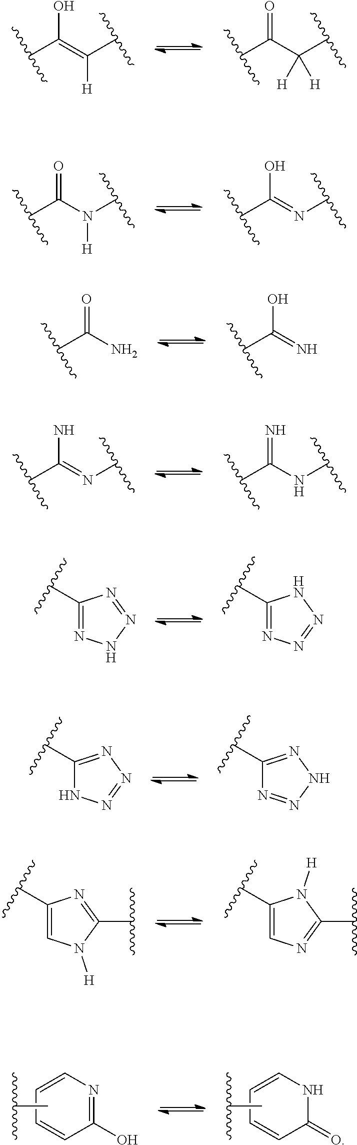

The compounds disclosed herein, in some embodiments, are used in different enriched isotopic forms, e.g., enriched in the content of ²H, ³H, C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion and metastasis formation are also termed malignant properties of cancers. These malignant properties differentiate cancers from benign tumors, which typically do not invade or metastasize.

In cancer, there are four general groups of tumor antigens: (i) viral tumor antigens which can be identical for any viral tumor of this type, (ii) carcinogenic tumor antigens which can be specific for patients and for the tumors, (iii) isoantigens of the transplantation type or tumor-specific transplantation antigens which can be different in all individual types of tumor but can be the same in different tumors caused by the same virus; and (iv) embryonic antigens.

As a result of the discovery of tumor antigens, tumor antigens have become important in the development of new cancer treatments that can specifically target the cancer. This has led to the development of antibodies directed against these tumor antigens.

In addition to the development of antibodies against tumor antigens for cancer treatment, antibodies that target immune cells to boost the immune response have also been developed. For example, an anti-CD40 antibody that is a CD40 agonist can be used to activate dendritic cells to enhance the immune response.

Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-βp, IFN-γ, GM-CSF, and LPS induced signaling events.

Humoral and cellular immune responses can be regulated, in part, by CD40. For example, in the absence of CD40 activation by its cognate binding partner, CD40 Ligand (CD40L), antigen presentation can result in tolerance. However, CD40 activation can ameliorate tolerance. In addition, CD40 activation can positively impact immune responses by enhancing antigen presentation by antigen presenting cells (APC), increasing cytokine and chemokine secretion, stimulating expression of and signaling by co-stimulatory molecules, and activating the cytolytic activity of different types of immune cells. Accordingly, the interaction between CD40 and CD40L can be essential to maintain proper humoral and cellular immune responses.

The intracellular effects of CD40 and CD40L interaction can include association of the CD40 cytoplasmic domain with TRAFs (TNF-R associated factors), which can lead to the activation of NFκB and Jun/AP1 pathways. While the response to activation of NFκB and Jun/AP1 pathways can be cell type-specific, often such activation can lead to increased production and secretion of cytokines, including IL-6, IL-8, IL-12, IL-15; increased production and secretion of chemokines, including MIP1α and β and RANTES; and increased expression of cellular adhesion molecules, including ICAM. While the effects of cytokines, chemokines and cellular adhesion molecules can be widespread, such effects can include enhanced survival and activation of T cells.

In addition to the enhanced immune responses induced by CD40 activation, CD40 activation can also be involved in chemokine- and cytokine-mediated cellular migration and differentiation; activation of immune cells, including monocytes; activation of and increased cytolytic activity of immune cells, including cytolytic T lymphocytes and natural killer cells; induction of CD40-positive tumor cell apoptosis and enhanced immunogenicity of CD40-positive tumors. In addition, CD40 can initiate and enhance immune responses by many different mechanisms, including, inducing antigen-presenting cell maturation and increased expression of costimulatory molecules, increasing production of and secretion of cytokines, and enhancing effector functions.

CD40 activation can be effective for inducing immune-mediated antitumor responses. For example, CD40 activation reverses host immune tolerance to tumor-specific antigens which leads to enhanced antitumor responses by T cells. Such antitumor activity can also occur in the absence of immune cells. Similarly, antitumor effects can occur in response to anti-CD40 antibody-mediated activation of CD40 and can be independent of or can involve antibody-dependent cellular cytotoxicity (ADCC). In addition to other CD40-mediated mechanisms of antitumor effects, CD40L-stimulation can cause dendritic cell maturation and stimulation. CD40L-stimulated dendritic cells can contribute to the antitumor response. Furthermore, vaccination strategies including CD40 can result in regression of CD40-positive and CD40-negative tumors.

CD40 activating antibodies (e.g., anti-CD40 activating monoclonal antibodies) can be useful for treatment of tumors. This can occur through one or more mechanisms, including cell activation, antigen presentation, production of cytokines and chemokines, amongst others. For example, CD40 antibodies activate dendritic cells, leading to processing and presentation of tumor antigens as well as enhanced immunogenicity of CD40-positive tumor cells. Not only can enhanced immunogenicity result in activation of CD40-positive tumor specific CD4⁺ and CD8⁺ T cells, but further antitumor activity can include, recruitment and activation monocytes, enhanced cytolytic activity of cytotoxic lymphocytes and natural killer cells as well as induction of apoptosis or by stimulation of a humoral response so as to directly target tumor cells. In addition, tumor cell debris, including tumor-specific antigens, can be presented to other cells of the immune system by CD40-activated antigen presenting cells.

Since CD40 can be important in an immune response, there is a need for enhanced CD40 meditated signaling events to provide reliable and rapid treatment options to patients suffering from diseases which may be ameliorated by treatment with CD40-targeted therapeutic strategies.

The CD40 mediated immune response can be further enhanced by targeting CD40 activation to the localized tumor site(s) through pairing with a tumor antigen binding domain. Such targeted CD40 activation and recruitment of immune cells to tumor cells may provide the advantage of maintaining therapeutic effectiveness with a lower dosage of a CD40 activating antibody construct or conjugate. A lower dosage may help mitigate any side effects of systemic CD40 activation such as cytokine release syndrome, which has been observed in some subjects treated with the agonistic CD40 monoclonal antibodies such as CP-870,893, dacetuzumab, Chi Lob 7/4, SEA-CD40, ADC-1013, 3C3, or 3G5. Systemic CD40 activation may also pose a risk of autoimmunity by causing APCs to break tolerance of autoantigens. For example, autoreactive T cells that manage to evade thymic selection may persist in the periphery in a state of tolerance against autoantigens, but CD40 activation can cause them to break tolerance and exhibit an autoimmune response. Accordingly, there is an important need for therapeutic, clinically relevant targeted CD40 activation that is enhanced at the localized tumor site relative to systemic activation. The presently described conjugate can be utilized as a safe and effective strategy to enhance the immune response. A conjugate can comprise an antigen binding domain and a CD40 binding domain, wherein the antigen binding domain specifically binds to a tumor antigen, wherein the CD40 binding domain comprises a CD40 agonist. This combination of a tumor antigen binding domain and a CD40 agonist can provide enhanced CD40 activation and recruitment of immune cells to the localized tumor site.

In addition to targeting immune cells to boost the immune response using anti-CD40 antibodies, antibodies that target other antigens expressed by immune cells have been developed. For example, an anti-DEC205 antibody, an anti-CD36 mannose scavenger receptor 1 antibody, an anti-CLEC9A antibody, CLEC12A, an anti-DC-SIGN antibody, an anti-BDCA-2 antibody, an anti-OX40L antibody, an anti-41BBL antibody, an anti-CD204 antibody, an anti-MARCO antibody, an anti-CLEC5A antibody, an anti-Dectin 1 antibody, and anti-Dectin 2 antibody, an anti-CLEC10A antibody, an anti-CD206 antibody, an anti-CD64 antibody, an anti-CD32A antibody, an anti-CD16A antibody, an anti-HVEM antibody, an anti-PD-L1 antibody, an anti-CD32B antibody or an anti-CD47 antibody can be used to target, respectively, surface DEC-205, CD36 mannose scavenger receptor 1, CLEC9A, CLEC12A, DC-SIGN, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B molecules expressed by antigen presenting cells or CD47 molecules expressed by T cells.

Cluster of Differentiation 205 (CD205 or DEC-205) is a member of the C-type multilectin family of endocytic receptors, which can include the macrophage mannose receptor (MMR) and the phospholipase A2 receptor (PLA₂R). DEC-205 can be a 205 kDa endocytic receptor highly expressed in cortical thymic epithelial cells, thymic medullary dendritic cells (CD11c⁺ CD8⁺), subpopulations of peripheral dendritic cells (CD11c⁺ CD8⁺). The DEC-205⁺ CD11c⁺ CD8⁺ dendritic cells (DCs) can function in cross-presentation of antigens derived from apoptotic cells. Additionally, DEC-205 can be significantly upregulated during DC maturation. DEC-205 can also be expressed at moderate levels in B cells and low levels in macrophages and T cells.

After antigen binding to DEC-205, the receptor-antigen complex can be internalized whereupon the antigen can be processed and be presented on the DC surface by a major histocompatibility complex class II (MHC II) or MHC class I. DEC-205 can deliver antigen to DCs for antigen presentation on MHC class II and cross-presentation on MHC class I. DEC-205 mediated antigen delivery for antigen presentation in DCs without an inflammatory stimulus can result in tolerance. Conversely, DEC-205 mediated antigen delivery in DCs in the presence of a maturational stimulus (e.g. a CD40 agonist) can result in long-term immunity via activation of antigen-specific CD4⁺ and CD8⁺ T cells.

CD36 mannose scavenger receptor 1 is an oxidized LDL receptor with two transmembrane domains located in the caveolae of the plasma membrane. It can be classified as a Class B scavenger receptor, which can be characterized by involvement in the removal of foreign substances and waste materials. This receptor can also be involved in cell adhesion, phagocytosis of apoptotic cells, and metabolism of long-chain fatty acids.

CLEC9A is a group V C-type lectin receptor. This receptor can be expressed as on myeloid lineage cells, and can be characterized as an activation receptor.

CLEC12A is a member of the C-type lectin/C-type lectin like domain super family that can be a negative regulator of granulocyte and monocyte function. It can also be involved in cell adhesion, cell-cell signaling, and glycoprotein turnover, and can play a role in the inflammatory response.

Dendritic cell-specific inter cellular adhesion molecule-3-grabbing non-integrin (DC-SIGN) or CD209, is a C-type lectin receptor that can be expressed on the surface of macrophages and dendritic cells. This receptor can recognize and bind to mannose type carbohydrates and be involved in activating phagocytosis, can mediate dendritic cell rolling, and can be involved in CD4+ T cell activation.

BDCA-2 is a C-type lectin that is a membrane protein of plasmacytoid dendritic cells. It can be involved in plasmacytoid dendritic cell function, such as ligand internalization and presentation.

OX40L, which can also be referred to as CD252, is the ligand for CD134 that can be expressed on dendritic cells. It can be involved in T cell activation.

41BBL, which can also be referred to as CD137L, is a member of the TNF superfamily, and can be expressed on B cells, dendritic cells, activated T cells, and macrophages. It can provide co-stimulatory signal for T cell activation and expansion.

CD204, which can also be referred to as macrophage scavenger receptor 1, is a macrophage scavenger receptor receptor. The gene for CD204 can encode three different class A macrophage scavenger receptor isoforms. The type 1 and type 2 isoforms can be involved in binding, internalizing, and processing negatively charged macromolecules, such as low density lipoproteins. The type 3 isoform can undergo altered intracellular processing in which it can be retained within the endoplasmic reticulum, and has been shown to have a dominant negative effect on the type 1 and type 2 isoforms.

Macrophage receptor with collagenous structure (MARCO), which can also be referred to as SCARA2, is a class A scavenger receptor with collagen-like and cysteine-rich domains. It can be expressed in macrophages, and can bind to modified low density lipoproteins. It can be involved in the removal of foreign substances and waste materials.

C-type lectin domain family 5 member A (CLEC5A) is a C-type lectin. It can be involved in the myeloid lineage activating pathway.

Dendritic cell-associated c-type lectin-1 (Dectin 1), which can also be referred to as CLEC7A, is member of the C-type lectin/C-type lectin-like super family. It can be expressed by myeloid dendritic cells, monocytes, macrophages, and B cells, and can be involved in antifungal immunity.

Dendritic cell-associated c-type lectin-2 (Dectin 2), which can also be referred to as CLEC6A, is member of the C-type lectin/C-type lectin-like super family. It can be expressed by dendritic cells, macrophages, monocytes and neutrophils. It can be involved in antifungal immunity.

CLEC10A, which can also be referred to as CD301, is member of the C-type lectin/C-type lectin-like super family. It can be expressed by dendritic cells, monocytes, and CD33+ myeloid cells, and can be involved in macrophage adhesion and migration.

CD206, which can also be referred to as macrophage mannose receptor, is a C-type lectin type I membrane glycoprotein. It can be expressed on dendritic cells, macrophages and endothelial cells, and can act as a pattern recognition receptor and bind high-mannose structures of viruses, bacteria, and fungi.

CD64, which can also be referred to as FcγRI, is a high affinity Fc receptor for IgG. It can be expressed by monocytes and macrophages. It can be involved in mediating phagocytosis, antigen capture, and antibody dependent cell-mediated cytoxicity.

CD32A, which can also be referred to as FcγRIIa, is a low affinity Fc receptor. It can be expressed on monocytes, granulocytes, B cells, and eosinophils. It can be involved in phagocytosis, antigen capture, and antibody dependent cell-mediated cytoxicity.

CD16A, which can also be referred to as FcγRIIIa, is low affinity Fc receptor. It can be expressed on NK cells, and can be involved in phagocytosis and antibody dependent cell-mediated cytotoxicity.

Herpesvirus entry mediator (HVEM), which can also be referred to as CD270, is a member of the TNF-receptor superfamily. It can be expressed on B cells, dendritic cells, T cells, NK cells, CD33+ myeloid cells, and monocytes. It can be involved in activating the immune response.

CD32B, which can also be referred to as FcγRIIb, is a low affinity Fc receptor. It can be expressed on B cells and myeloid dendritic cells. It can be involved in inhibiting maturation and cell activation of dendritic cells.

The HER2/neu (human epidermal growth factor receptor 2/receptor tyrosine-protein kinase erbB-2) is part of the human epidermal growth factor family. Overexpression of this protein can be shown to play an important role in the progression of cancer, for example, breast cancer. The HER2/neu protein can function as a receptor tyrosine kinase and autophosphorylates upon dimerization with binding partners. HER2/neu can activate several signaling pathways including, for example, mitogen-activated protein kinase, phosphoinositide 3-kinase, phospholipase Cγ, protein kinase C, and signal transducer and activator of transcription (STAT). Examples of antibodies that can target and inhibit HER2/neu can include trastuzumab and pertuzumab.

EGFR (epidermal growth factor receptor) encodes a member of the human epidermal growth factor family. Mutations that can lead to EGFR overexpression or over activity can be associated with a number of cancers, including squamous cell carcinoma and glioblastomas. EGFR can function as a receptor tyrosine kinase and ligand binding can trigger dimerization with binding partners and autophosphorylation. The phosphorylated EGFR can then activate several downstream signaling pathways including mitogen-activated protein kinase, phosphoinositide 3-kinase, phospholipase Cγ, protein kinase C, and signal transducer and activator of transcription (STAT). Examples of antibodies that can target and inhibit EGFR can include cetuximab, panutumumab, nimotuzumab, and zalutumumab. One mutant variant of EGFR is EGFRvIII (epidermal growth factor receptor variant III). EGFRvIII can be the result of an EGFR gene rearrangement in which exons 2-7 of the extracellular domain are deleted. This mutation can result in a mutant receptor incapable of binding to any known ligand. The resulting receptor can engage in a constitutive low-level signaling and can be implicated in tumor progression. Examples of antibodies that can target EGFRvIII can include AMG595 and ABT806.

C-Met (hepatocyte growth factor receptor) encodes a member of the receptor tyrosine kinase family of proteins. C-Met overexpression and over activity can be implicated in various cancers including lung adenocarcinomas, and high c-Met levels can be associated with poor patient outcome. Binding of hepatocyte growth factor can induce dimerization and autophosphorylation of c-Met. The c-Met receptor can activate various downstream signaling pathways including mitogen-activated protein kinase, phosphoinositide 3-kinase, and protein kinase C pathways. The antibody onartuzumab can target and inhibit c-Met.

HER3 (human epidermal growth factor receptor 3) encodes a member of the human epidermal growth factor receptor family. Ligand binding can induce dimerization and autophosphorylation of cytoplasmic tyrosine residues that then can recruit signaling proteins for downstream signaling pathway activation including mitogen-activated protein kinase and phosphoinoside 3-kinase pathways. HER3 can play an active role in cell proliferation and survival, and can be overexpressed, overactive, and/or mutated in various cancers. For example, HER3 can be overexpressed in breast, ovarian, prostate, colon, pancreas, stomach, oral, and lung cancers. The antibody patritumab can target and inhibit HER3.

MUC1 (mucin 1, cell surface associated) encodes a member of the mucin family of glycosylated proteins that can play an important role in cell adhesion and forming protective mucosal layers on epithelial surfaces. MUC1 can be proteolytically cleaved into alpha and beta subunits that form a heterodimeric complex with the N-terminal alpha subunit providing cell-adhesion functionality and the C-terminal beta subunit modulating cell signaling pathways including the mitogen activated map kinase pathway. MUC1 can play a role in cancer progression, for example, by regulating TP53-mediated transcription. MUC1 overexpression, aberrant intracellular localization, and glycosylation changes can all be associated with carcinomas including pancreatic cancer cells. The antibody clivatuzumab can target MUC1.

MUC16 (mucin 16, cell surface associated) encodes the largest member of the mucin family of glycosylated proteins that can play an important role in cell adhesion and forming protective mucosal layers on epithelial surfaces. MUC16 can be a highly glycosylated 2.5 MDa transmembrane protein that can provide a hydrophilic lubricating barrier on epithelial cells. The cytoplasmic tail of MUC16 can be involved with various signaling pathways including the JAK2-STAT3 and Src kinase pathways. A peptide epitope of MUC16 can be used as biomarker for detecting ovarian cancer. Elevated expression of MUC16 can be present in advanced ovarian cancers and pancreatic cancers. The antibody sofituzumab can target MUC16.

EPCAM (epithelial cell adhesion molecule) encodes a transmembrane glycoprotein that can be frequently and highly expressed in carcinomas and tumor-initiating cells. EPCAM can also be a pluripotent stem cell marker. EPCAM can modulate a variety of pathways including cell-cell adhesion, cellular proliferation, migration, invasion, maintenance of a pluripotent state, and differentiation in the context of tumor cells. The antibodies edrecolomab and adecatumumab can target EPCAM.

MSLN (mesothelin) encodes a 40 kDa cell GPI-anchored membrane surface protein believed to function in cell adhesion. MSLN is overexpressed in mesothelioma and certain types of pancreatic, lung, and ovarian cancers. MSLN-related peptides that circulate in serum of patients suffering from pleural mesothelioma are used as biomarkers for monitoring the disease. MSLN may promote metastasis by inducing matrix metalloproteinase 7 and 9 expression. The monoclonal antibody anetumab has been developed to target MSLN.

CA6 (carbonic anhydrase VI) encodes one of several isozymes of carbonic anhydrase. CA6 is found in salivary glands and may play a role in the reversible hydration of carbon dioxide. CA6 is expressed in human serous ovarian adenocarcinomas. The monoclonal antibody huDS6 has been developed to target CA6.

NAPI2B (sodium/phosphate cotransporter 2B) encodes a type II sodium-phosphate cotransporter. NAPI2B is highly expressed on the tumor surface in lung, ovarian, and thyroid cancers as well as in normal lung pneumocytes. The monoclonal antibody lifastuzumab has been developed to target NAPI2B.

TROP2 (trophoblast antigen 2) encodes a transmembrane glycoprotein that acts as an intracellular calcium signal transducer. TROP2 binds to multiple factors such as IGF-1, claudin-1, claudin-7, cyclin Dl, and PKC. TROP2 including intracellular calcium signaling and the mitogen activated protein kinase pathway. TROP 2 plays a role in cell self-renewal, proliferation, invasion, and survival. Discovered first in trophoblast cells that have the ability to invade uterine decidua during placental implantation, TROP2 overexpression has been shown to be capable of stimulating cancer growth. TROP2 overexpression has been observed in breast, cervix, colorectal, esophagus, lung, non-Hodgkin's lymphoma, chronic lymphocytic lymphoma, Raji Burkitt lymphoma, oral squamous cell, ovarian, pancreatic, prostate, stomach, thyroid, urinary bladder, and uterine carcinomas. The monoclonal antibody sactuzumab has been developed to target TROP2.

CEA (carcinoembryonic antigen) encodes a family of related glycoproteins involved in cell adhesion. CEA is a biomarker for gastrointestinal cancers and may promote tumor development by means of its cell adhesion function. CEA levels have been found to be elevated in serum of individuals with colorectal carcinoma. CEA levels have also been found to be elevated in gastric carcinoma, pancreatic carcinoma, lung carcinoma, breast carcinoma, and medullary thyroid carcinoma. The monoclonal antibodies PR1A3 and Ab2-3 have been developed to target CEA.

CLDN18.2 (claudin 18) encodes a member of the claudin family of integral membrane proteins. CLDN18.2 is a component of tight junctions that create a physical barrier to prevent diffusion of solutes and water through the paracellular space between epithelial cells. CLDN18.2 is overexpressed in infiltrating ductal adenocarcinomas, but is reduced in some gastric carcinomas. The monoclonal antibody claudiximab has been developed to target CLDN18.2.

FAP (fibroblast activation protein, alpha) encodes a homodimeric integral membrane protein from a family of serine proteases. FAP is believed to play a role in many processes including tissue remodeling, fibrosis, wound healing, inflammation, and tumor growth. FAP enhances tumor growth and invasion by promoting angiogenesis, collagen fiber degradation and apoptosis, and by downregulating the immune response. FAP is selectively expressed on fibroblasts within the tumor stroma. The monoclonal antibody sibrotuzumab has been developed to target FAP.

EphA2 (EPH Receptor A2) encodes a member of the ephrin receptor subfamily of the protein-tyrosine kinase family. EphA2 binds to ephrin-A ligands. Activation of EphA2 receptor upon ligand binding can result in modulation of migration, integrin-mediated adhesion, proliferation, and differentiation. EphA2 is overexpressed in various cancers including breast, prostate, urinary bladder, skin, lung, ovarian, and brain cancers. High EphA2 expression is also correlated with poor prognosis. The monoclonal antibodies DS-8895a opt1, DS-8895 opt2, and Anti-EphA2 of MEDI-547 have been developed to target EphA2.

RON (macrophage stimulating 1 receptor) encodes a cell surface receptor for macrophage stimulating protein (MSP) with tyrosine kinase activity and belongs to the MET proto-oncogene family. RON has significant structural similarity and sequence homology with the cancer-related gene C-MET. RON plays a significant role in KRAS oncogene addiction and has also been shown to be overexpressed in pancreatic cancers. Altered Ron expression and activation has been associated with decreased survival and cancer progression in various cancers including gastric, colon, breast, bladder, renal cell, ovarian, and hepatocellular cancers. The monoclonal antibody narnatumab has been developed to target RON.

LY6E (lymphocyte antigen 6 complex, locus E) encodes an interferon alpha-inducible GPI-anchored cell membrane protein. LY6E is overexpressed in numerous cancers including lung, gastric, ovarian, breast, kidney, pancreatic, and head and neck carcinomas. The monoclonal antibody RG7841 has been developed to target LY6E.

FRA (folate receptor alpha) encodes a GPI-anchored cell surface glycoprotein. FRA binds folic acid, a molecule needed for cell growth and DNA synthesis, and mediates its internalization via receptor-mediated endocytosis. FRA is overexpressed in various cancers including prostate, breast, ovarian, pancreatic, mesothelioma, non-small cell lung carcinoma, and head and neck cancer. FRA expression has also been found to enhance tumor cell proliferation. The monoclonal antibodies farletuzumab and mirvetuximab have been developed to target FRA.

PSMA (prostate specific membrane antigen) is a type II transmembrane glycoprotein belonging to the M28 peptidase family that is expressed in all types of prostate tissues. PSMA is upregulated in cancer cells within the prostate and is used as a marker for prostate cancer. PSMA expression may also serve as a predictor of disease recurrence in prostate cancer patients. The monoclonal antibodies J591 variant 1 and J591 variant 2 have been developed to target PSMA.

DLL3 (delta-like 3) encodes a ligand in the Notch signaling pathway that is associated with neuroendocrine cancer. DLL3 is most highly expressed in the fetal brain and is involved in somitogenesis in the paraxial mesoderm. DLL3 is expressed on tumor cell surfaces but not in normal tissues. The monoclonal antibody rovalpituzumab has been developed to target DLL3.

PTK7 (tyrosine protein kinase-like 7) encodes a receptor tyrosine kinase that lacks catalytic tyrosine kinase activity but is nevertheless capable of signal transduction. PTK7 interacts with the WNT signaling pathway, which itself has important roles in epithelial mesenchymal transition and various cancers such as breast cancer. PTK7 overexpression has been associated with patient prognosis depending on the cancer type. The monoclonal antibodies PF-06647020 and the anti-PTK7 antibody described by SEQ ID NO 440 and 445 have been developed to target PTK7.

LIV1 (LIV-1 protein, estrogen regulated) encodes a member of the LIV-1 subfamily of ZIP (Zrt-, Irt-like proteins) zinc transporters. LIV1 is an estrogen regulated protein that transports zinc and/or other ions across the cell membrane. Elevated levels of LIV1 have been shown in estrogen receptor positive breast cancers, and LIV1 is used as a marker of ER-positive cancers. LIV1 has also been implicated as a downstream target of the STAT3 transcription factor and as playing an essential role in the nuclear localization of the Snail transcription factor that modulates epithelial-to-mesenchymal transition. The monoclonal antibody Ladiratuzumab has been developed to target LIV1.

ROR1 (receptor tyrosine kinase-like orphan receptor 1) encodes a member of the ROR family of orphan receptors. ROR1 has been found to bind Wnt5a, a non-canonical Wnt via a Frizzled domain (FZD), and plays an important role in skeletal, cardiorespiratory, and neurological development. ROR1 expression is predominantly restricted to embryonic development and is absent in most mature tissues. In contrast, ROR1 expression is upregulated in B-Cell chronic lymphocytic leukemia, acute lymphocytic leukemia, non-Hodgkin lymphoma, and myeloid malignancies. The monoclonal antibody cirmtuzumab has been developed to target ROR1.

MAGE-A3 (melanoma-associated antigen 3) encodes a member of the melanoma-associated antigen gene family. The function of MAGE-A3 is not known, but its elevated expression has been observed in various cancers including melanoma, non-small cell lung cancer, and in putative cancer stem cell populations in bladder cancer. The monoclonal antibody described by SEQ ID NO 479 and 484 has been developed to target MAGE-A3.

NY-ESO-1 (New York esophageal squamous cell carcinoma 1) encodes a member of the cancer-testis family of proteins. Cancer-testis antigen expression is normally restricted to testicular germ cells in adult tissues, but has been found to be aberrantly expressed in various tumors including soft tissue sarcomas, melanoma, epithelial cancers, and myxoid and round cell liposarcomas. The monoclonal antibody described by SEQ ID NO 492 and 497 has been developed to target NY-ESO-1.

Immune-stimulatory molecular motifs, such as Pathogen-Associated Molecular Pattern molecules (PAMPs), can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens. Similar to other protein families, there are many different TLRs, including TLR4, TLR7 and TLR8. Several agonists targeting activation of different TLRs have been tried in various immunotherapies, including vaccine adjuvants and in cancer immunotherapies. However, therapeutic use of PAMPs and DAMPs or other mechanisms of intervention can be limited because systemic activation of PAMP and DAMP signaling pathways can have life-threatening consequences due to cytokine syndrome-induced or cytokine storm-induced toxic shock syndrome. Accordingly, there is a critical need for therapeutic, clinically relevant targeted delivery of PAMP and DAMP agonists for safe and effective strategies to enhance immune responses.

The presently described antibody constructs and conjugates can be utilized as strategy to enhance immune responses. A conjugate can comprise an antibody construct and an immune-stimulatory compound. A conjugate can comprise a first binding domain, a second binding domain, and an immune-stimulatory compound. A conjugate can comprise a first binding domain, a second binding domain, an Fc domain, and an immune-stimulatory compound. An antibody construct can comprise a first binding domain, a second binding domain, and a third binding domain. An antibody construct can comprise a first binding domain, a second binding domain, a third binding domain, and an Fc domain, wherein the first binding domain is attached to the Fc domain, wherein the second binding domain is attached to the Fc domain, and wherein the third binding domain is attached to a C-terminal end of a light chain of the first binding domain. A conjugate can comprise a first binding domain, a second binding domain, a third binding domain, and an immune-stimulatory compound. A conjugate can comprise a first binding domain, a second binding domain, a third binding domain, an Fc domain, and an immune-stimulatory compound, wherein the first binding domain is attached to the Fc domain, wherein the second binding domain is attached to the Fc domain, and wherein the third binding domain is attached to a C-terminal end of a light chain of the first binding domain. A conjugate can comprise a first binding domain, a second binding domain, a third binding domain, an Fc domain, and an immune-stimulatory compound, wherein the first binding domain is attached to the Fc domain, wherein the second binding domain is attached to the Fc domain, and wherein the third binding domain is attached to a C-terminal end of a light chain of the first binding domain.

### Binding Domains

A conjugate or antibody construct can contain one or more binding domains. A conjugate or antibody construct can comprise a first binding domain. A conjugate or antibody construct can comprise a second binding domain. A binding domain can specifically bind to a molecule on a cell surface or a fragment thereof. A binding domain can specifically bind to an antigen on a cell surface, for example, of a tumor cell, of an antigen presenting cell such as a dendritic cell or macrophage or other immune cell cell such as a T cell. In some embodiments, an immune cell is a T cell, B cell, NK cell, or NKT cell. In some embodiments, an immune cell is an antigen presenting cell. In some embodiments, an immune cell is not an antigen presenting cell. A binding domain can specifically bind to a molecule, wherein the molecule comprises an antigen. A binding domain can be a cell surface receptor agonist. A binding domain can be an antigen binding domain. An antigen binding domain can be a cell surface receptor agonist. An antigen binding domain can be a domain that can specifically bind to an antigen. An antigen binding domain can specifically bind to a tumor antigen. An antigen binding domain can be an antigen-binding portion of an antibody or an antibody fragment. An antigen binding domain can be one or more fragments of an antibody that can retain the ability to specifically bind to an antigen. An antigen binding domain can be any antigen binding fragment. An antigen binding domain can recognize a single antigen. A conjugate can comprise, for example, two, three, four, five, six, seven, eight, nine, ten, or more antigen binding domains. A conjugate or antibody construct can comprise two antigen binding domains in which each antigen binding domain can recognize the same antigen. A conjugate or antibody construct can comprise two antigen binding domains in which each antigen binding domain can recognize different antigens. A conjugate or antibody construct can comprise three antigen binding domains in which each antigen binding domain can recognize different antigens. A conjugate or antibody construct can comprise three antigen binding domains in which two of the antigen binding domains can recognize the same antigen. An antigen binding domain can be in a scaffold, in which a scaffold is a supporting framework for the antigen binding domain. An antigen binding domain can be in a non-antibody scaffold. An antigen binding domain can be in an antibody scaffold or antibody-like scaffold. A conjugate or antibody construct can comprise an antigen binding domain in a scaffold. The conjugate or antibody construct can comprise an Fc fusion protein product (also referred to as a fusion peptide). In some embodiments, the antibody construct is a fusion peptide or the antibody construct of a conjugate is a fusion peptide. For example, an antigen binding domain and an Fc domain can be expressed as fusion peptide. Two antigen binding domains and an Fc domain can be expressed as a fusion peptide.

The conjugates or antibody constructs described herein can comprise a binding domain that can specifically bind to a tumor antigen. A tumor antigen can be a tumor specific antigen and/or a tumor associated antigen. As described herein, a “tumor antigen” can refer to a molecular marker that can be expressed by a neoplastic tumor cell and/or within a tumor microenvironment. The molecular marker can be a cell surface receptor. For example, a tumor associated antigen can be an antigen expressed on a cell associated with a tumor, such as a neoplastic cell, stromal cell, endothelial cell, fibroblast, or tumor-infiltrating immune cell. For example, the tumor associated antigen Her2/Neu can be overexpressed by certain types of breast and ovarian cancer. A tumor antigen can also be ectopically expressed by a tumor and contribute to deregulation of the cell cycle, reduced apoptosis, metastasis, or escape from immune surveillance. Tumor associated antigens can generally be proteins or polypeptides derived therefrom, but can be glycans, lipids, or other small organic molecules. Additionally, a tumor antigen can arise through increases or decreases in post-translational processing exhibited by a cancer cell compared to a normal cell, for example, protein glycosylation, protein lipidation, protein phosphorylation, or protein acetylation.

In certain embodiments, a binding domain specifically can bind to a tumor associated antigen selected from the following: CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), ferritin, GD2, GD3, GM2, Ley, CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, Her-2/neu, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe(animal), CYP1B1, PLAV1, BORIS, Tn, GloboH, ETV6-AML, NY-BR-1, RGS5, SART3, STn, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, C1orf186, Fos-related antigen 1, VEGFR1, endoglin, PD-L, VTCN1 (B7-H4), VISTA, or a fragment thereof. In certain embodiments, a binding domain specifically binds to a tumor associated antigen comprising Her2/Neu (CD340).

In certain embodiments, a binding domain specifically can bind to a tumor associated antigen comprising GD2, GD3, GM2, Le^{y}, polysialic acid, fucosyl GM1, GM3, Tn, STn, sLe(animal), or GloboH. In certain embodiments, a binding domain specifically can bind to a tumor associated antigen comprising at least 80%, 90%, 95%, 97%, 98%, 99% or 100% homology to the amino acid sequence of CD5, CD19, CD20, CD25, CD37, CD30, CD33, CD45, CAMPATH-1, BCMA, CS-1, PD-L, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), ferritin, CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, Her-2/neu, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, PD-L1, VTCN1 (B7-H4), VISTA, or a fragment thereof. In certain embodiments, a binding domain specifically binds to a tumor associated antigen comprising Her2/Neu (CD340). TABLE 1 below shows some exemplary amino acid sequences of these tumor antigens.

TABLE 1
Exemplary Tumor Antigen Amino Acid Sequences

| | SEQ ID |
|---|---|
| Antigen | NO: |
| HER2 (isoform 1) | 850 |
| HER2 (isoform 2) | 851 |
| HER2 (isoform 3) | 852 |
| HER2 (isoform 4) | 853 |
| HER2 (isoform 5) | 854 |
| HER2 (isoform 6) | 855 |
| EGFR (isoform 1) | 856 |
| EGFR (isoform 2) | 857 |
| EGFR (isoform 3) | 858 |
| EGFR (isoform 4) | 859 |
| CMET (isoform 1) | 860 |
| CMET (isoform 2) | 861 |
| CMET (isoform 3) | 862 |
| HER3 (isoform 1) | 863 |
| HER3 (isoform 2) | 864 |
| HER3 (isoform 3) | 865 |
| HER3 (isoform 4) | 866 |
| HER3 (isoform 5) | 867 |
| MUC1 (isoform 1) | 868 |
| MUC1 (Isoform 2) | 869 |
| MUC1 (Isoform 3) | 870 |
| MUC1 (Isoform 4) | 871 |
| MUC1 (Isoform 5) | 872 |
| MUC1 (Isoform 6) | 873 |
| MUC1 (Isoform Y) | 874 |
| MUC1 (Isoform 8) | 875 |
| MUC1 (Isoform 9) | 876 |
| MUC1 (Isoform F) | 877 |
| MUC1 (Isoform Y- | 878 |
| LSP) | |
| MUC1 (Isoform | 879 |
| S2) | |
| MUC1 (Isoform | 880 |
| M6) | |
| MUC1 (Isoform | 881 |
| ZD) | |
| MUC1 (Isoform | 882 |
| T10) | |
| MUC1 (Isoform | 883 |
| E2) | |
| MUC1 (Isoform | 884 |
| J13) | |
| MUC16 | 885 |
| EPCAM | 886 |
| MSLN (isoform | 887 |
| Q13421-1) | |
| MSLN (isoform 2) | 888 |
| MSLN (isoform 3) | 889 |
| MSLN (isoform 4) | 890 |
| CA6 (isoform 1) | 891 |
| CA6 (isoform 2) | 892 |
| CA6 (isoform 3) | 893 |
| NAPI2B (isoform | 894 |
| 1) | |
| NAPI2B (isoform | 895 |
| 2) | |
| TROP2 | 896 |
| CEA (CEACAM5, | 897 |
| isoform 1) | |
| CEA (CEACAM5, | 898 |
| isoform 2) | |
| CLDN18.2 | 899 |
| (isoform A2) | |
| EGFRvIII | 900 |
| FAP (isoform 1) | 901 |
| FAP (isoform 2) | 902 |
| EphA2 (isoform 1) | 903 |
| EphA2 (isoform 2) | 904 |
| RON (isoform | 905 |
| RON) | |
| RON (isoform | 906 |
| delta) | |
| RON (isoform | 907 |
| RON-1) | |
| RON (isoform | 908 |
| RON-2) | |
| RON (isoform | 909 |
| RON-3) | |
| RON (isoform | 910 |
| RON-4) | |
| LY6E | 911 |
| FRA1 (isoform 1) | 912 |
| FRA1 (isoform 2) | 913 |
| PSMA (isoform 1) | 914 |
| PSMA (isoform | 915 |
| PMSA-3) | |
| PSMA (isoform | 916 |
| PMSA-4) | |
| PSMA (isoform | 917 |
| PMSA′) | |
| PSMA (isoform | 918 |
| PSMA-7) | |
| PSMA (isoform | 919 |
| PSMA-8) | |
| PSMA (isoform | 920 |
| PSMA-9) | |
| PSMA (isoform 10) | 921 |
| DLL3 (isoform 1) | 922 |
| DLL3 (isoform 2) | 923 |
| PTK7 (isoform 1) | 924 |
| PTK7 (isoform 2) | 925 |
| PTK7 (isoform 3) | 926 |
| PTK7 (isoform 4) | 927 |
| PTK7 (isoform 5) | 928 |
| PTK7 (isoform 6) | 929 |
| LIV1 (isoform 1) | 930 |
| LIV1 (isoform 2) | 931 |
| ROR1 (isoform 1) | 932 |
| ROR1 (isoform | 933 |
| short) | |
| ROR1 (isoform 3) | 934 |
| MAGE-A3 | 935 |
| NY-ESO-1 | 936 |
| LRRC15 (isoform | 937 |
| 1) | |
| LRRC15 (isoform | 938 |
| 2) | |
| GLP-3 (GPC3) | 939 |
| (isoform 1) | |
| GLP-3 (GPC3) | 940 |
| (isoform 2) | |
| GLP-3 (GPC3) | 941 |
| (isoform 3) | |
| CLDN6 | 942 |
| CLDN16 | 943 |
| UPK1B | 944 |
| VTCN1 | 945 |
| STRA6 (isoform 1) | 946 |
| STRA6 (isoform 2) | 947 |
| STRA6 (isoform 3) | 948 |
| STRA6 (isoform 4) | 949 |
| STRA6 (isoform 5) | 950 |
| STRA6 (isoform 6 | 951 |
| CD273 (isoform 1) | 952 |
| CD273 (isoform 2) | 953 |
| CD273 (isoform 3) | 954 |
| PD-L1 (isoform 1) | 955 |
| PD-L1 (isoform 2) | 956 |
| PD-L1 (isoform 3) | 957 |
| CD5 | 1030 |
| CD19 (isoform 1) | 1031 |
| CD19 (isoform 2) | 1032 |
| CD20 (isoform 1) | 1033 |
| CD20 (isoform 2) | 1034 |
| CD25 | 1035 |
| CD37 (isoform 1) | 1036 |
| CD37 (isoform 2) | 1037 |
| CD37 (isoform 3) | 1038 |
| CD30 (isoform 1) | 1039 |
| CD30 (isoform 2) | 1040 |
| CD30 (isoform 3) | 1041 |
| CD33 (isoform 1) | 1042 |
| CD33 (isoform 2) | 1043 |
| CD33 (isoform 3) | 1044 |
| CD45 (isoform 1) | 1045 |
| CD45 (isoform 2) | 1046 |
| CAMPTH-1 | 1047 |
| BCMA (isoform 1) | 1048 |
| BCMA (isoform 2) | 1049 |
| CS-1 (isoform 1) | 1050 |
| CS-1 (isoform 2) | 1051 |
| CS-1 (isoform 3) | 1052 |
| B7-H3 (isoform 1) | 1053 |
| B7-H3 (isoform 2) | 1054 |
| B7-H3 (isoform 3) | 1055 |
| B7-H3 (isoform 4) | 1056 |
| B7-DC (isoform 1) | 1057 |
| B7-DC (isoform 2) | 1058 |
| B7-DC (isoform 3) | 1059 |
| HLA-DR (isoform | 1060 |
| 1) | |
| HLA-DR (isoform | 1061 |
| 2) | |
| HLA-DR (isoform | 1062 |
| 3) | |
| MUC15 (isoform | 1063 |
| 1) | |
| MUC15 (isoform | 1064 |
| 2) | |
| Folate-Binding | 1065 |
| Protein | |
| A33 | 1066 |
| G250 | 1067 |
| Ferritin light chain | 1068 |
| Ferritin heavy | 1069 |
| chain | |
| EGF (isoform 1) | 1070 |
| EGF (isoform 2) | 1071 |
| EGF (isoform 3) | 1072 |
| IL-2 Receptor | 1073 |
| (gamma subunit) | |
| (isoform 1) | |
| IL-2 Receptor | 1074 |
| (gamma subunit) | |
| (isoform 2) | |
| IL-2 Receptor | 1075 |
| (alpha subunit) | |
| Tenascin (isoform | 1076 |
| 1) | |
| Tenascin (isoform | 1077 |
| 2) | |
| Tenascin (isoform | 1078 |
| 3) | |
| Tenascin (isoform | 1079 |
| 4) | |
| Tenascin (isoform | 1080 |
| 5) | |
| Tenascin (isoform | 1081 |
| 6) | |
| Endosialin (isoform | 1082 |
| 1) | |
| Endosialin (isoform | 1083 |
| 2) | |
| Vascular | 1084 |
| endothelial growth | |
| factor (isoform | |
| VEGF206) | |
| Vascular | 1085 |
| endothelial growth | |
| factor (isoform | |
| VEGF189) | |
| Vascular | 1086 |
| endothelial growth | |
| factor (isoform | |
| VEGF183) | |
| Vascular | 1087 |
| endothelial growth | |
| factor (isoform | |
| VEGF165) | |
| Vascular | 1088 |
| endothelial growth | |
| factor (isoform | |
| VEGF148) | |
| Vascular | 1089 |
| endothelial growth | |
| factor (isoform | |
| VEGF145) | |
| Vascular | 1090 |
| endothelial growth | |
| factor (isoform | |
| VEGF165B) | |
| Vascular | 1091 |
| endothelial growth | |
| factor (isoform | |
| VEGF121) | |
| Vascular | 1092 |
| endothelial growth | |
| factor (isoform | |
| VEGF111) | |
| Vascular | 1093 |
| endothelial growth | |
| factor (isoform L- | |
| VEGF165) | |
| Vascular | 1094 |
| endothelial growth | |
| factor (isoform L- | |
| VEGF121) | |
| Vascular | 1095 |
| endothelial growth | |
| factor (isoform L- | |
| VEGF189) | |
| Vascular | 1096 |
| endothelial growth | |
| factor (isoform L- | |
| VEGF206) | |
| Vascular | 1097 |
| endothelial growth | |
| factor (isoform 15) | |
| Vascular | 1098 |
| endothelial growth | |
| factor (isoform 16) | |
| Vascular | 1099 |
| endothelial growth | |
| factor (isoform 17) | |
| Vascular | 1100 |
| endothelial growth | |
| factor (isoform 18) | |
| Integrin alpha V | 1101 |
| (isoform 1) | |
| Integrin alpha V | 1102 |
| (isoform 2) | |
| Integrin alpha V | 1103 |
| (isoform 3) | |
| Integrin beta 3 | 1104 |
| (isoform Beta-3A) | |
| Integrin beta 3 | 1105 |
| (isoform Beta-3B) | |
| Integrin beta 3 | 1106 |
| (isoform Beta-3C) | |
| WT1 (isoform 1) | 1107 |
| WT1 (isoform 2) | 1108 |
| WT1 (isoform 3) | 1109 |
| WT1 (isoform 4) | 1110 |
| WT1 (isoform 6) | 1111 |
| WT1 (isoform 7) | 1112 |
| WT1 (isoform 8) | 1113 |
| WT1 (isoform 9) | 1114 |
| LMP2 (isoform | 1115 |
| LMP2.L) | |
| LMP2 (isoform | 1116 |
| LMP2.S) | |
| HPV E6 (strain 16) | 1117 |
| HPV E6 (strain 18) | 1118 |
| HPV E7 (strain 16) | 1119 |
| HPV E7 (strain 18) | 1120 |
| P53 nonmutant | 1121 |
| (isoform 1) | |
| P53 nonmutant | 1122 |
| (isoform 2) | |
| P53 nonmutant | 1123 |
| (isoform 3) | |
| P53 nonmutant | 1124 |
| (isoform 4) | |
| P53 nonmutant | 1125 |
| (isoform 5) | |
| P53 nonmutant | 1126 |
| (isoform 6) | |
| P53 nonmutant | 1127 |
| (isoform 7) | |
| P53 nonmutant | 1128 |
| (isoform 8) | |
| P53 nonmutant | 1129 |
| (isoform 9) | |
| MalenA/MART1 | 1130 |
| Gp100 (isoform 1) | 1131 |
| Gp100 (isoform 2) | 1132 |
| Gp100 (isoform 3) | 1133 |
| Gp100 (isoform 4) | 1134 |
| Gp100 (isoform 5) | 1135 |
| PR1 (TMEM37) | 1136 |
| BCR-abl (isofrom | 1137 |
| X3) | |
| BCR-abl (isofrom | 1138 |
| Y5) | |
| BCR-abl (isofrom | 1139 |
| X9) | |
| BCR-abl (isofrom | 1140 |
| X2) | |
| BCR-abl (isofrom | 1141 |
| e8a2 variant) | |
| BCR-abl (isofrom | 1142 |
| Y3) | |
| BCR-abl (isofrom | 1143 |
| Y2) | |
| BCR-abl (isofrom | 1144 |
| Y1) | |
| BCR-abl (isofrom | 1145 |
| X6) | |
| BCR-abl (isofrom | 1146 |
| Y4) | |
| BCR-abl (isofrom | 1147 |
| Y6) | |
| Tyrosinase | 1148 |
| (isoform 1) | |
| Tyrosinase | 1149 |
| (isoform 2) | |
| Survivin (isoform | 1150 |
| 1) | |
| Survivin (isoform | 1151 |
| 2) | |
| Survivin (isoform | 1152 |
| 3) | |
| Survivin (isoform | 1153 |
| 4) | |
| Survivin (isoform | 1154 |
| 5) | |
| Survivin (isoform | 1155 |
| 6) | |
| Survivin (isoform | 1156 |
| 7) | |
| PSA (isoform 1) | 1157 |
| PSA (isoform 2) | 1158 |
| PSA (isoform 3) | 1159 |
| PSA (isoform 4) | 1160 |
| PSA (isoform 5) | 1161 |
| hTERT (isoform 1) | 1162 |
| hTERT (isoform 2) | 1163 |
| hTERT (isoform 3) | 1164 |
| hTERT (isoform 4) | 1165 |
| Sarcoma | 1166 |
| translocation | |
| breakpoint fusion | |
| protein (Ewing | |
| sarcome breakpoint | |
| region 1 protein) | |
| (isoform EWS) | |
| Sarcoma | 1167 |
| translocation | |
| breakpoint fusion | |
| protein (Ewing | |
| sarcome breakpoint | |
| region 1 protein) | |
| (isoform EWS-B) | |
| Sarcoma | 1168 |
| translocation | |
| breakpoint fusion | |
| protein (Ewing | |
| sarcome breakpoint | |
| region 1 protein) | |
| (isoform 3) | |
| Sarcoma | 1169 |
| translocation | |
| breakpoint fusion | |
| protein (Ewing | |
| sarcome breakpoint | |
| region 1 protein) | |
| (isoform 4) | |
| Sarcoma | 1170 |
| translocation | |
| breakpoint fusion | |
| protein (Ewing | |
| sarcome breakpoint | |
| region 1 protein) | |
| (isoform 5) | |
| Sarcoma | 1171 |
| translocation | |
| breakpoint fusion | |
| protein (Ewing | |
| sarcome breakpoint | |
| region 1 protein) | |
| (isoform 6) | |
| PAP (isoform 1) | 1172 |
| PAP (isoform 2) | 1173 |
| PAP (isoform 3) | 1174 |
| ML-IAP (isoform | 1175 |
| 2) | |
| ML-IAP (isoform | 1176 |
| 1) | |
| AFP | 1177 |
| ERG (isoform | 1178 |
| ERG-3) | |
| ERG (isoform | 1179 |
| ERG-2) | |
| ERG (isoform | 1180 |
| ERG-1) | |
| ERG (isoform 5) | 1181 |
| ERG (isoform 7) | 1182 |
| ERG (isoform 8) | 1183 |
| NA17 | 1184 |
| PAX3 (isoform | 1185 |
| Pax3) | |
| PAX3 (isoform | 1186 |
| Pax3A) | |
| PAX3 (isoform | 1187 |
| Pax3B) | |
| PAX3 (isoform | 1188 |
| Pax3G) | |
| PAX3 (isoform | 1189 |
| Pax3H) | |
| PAX3 (isoform 6) | 1190 |
| PAX3 (isoform 7) | 1191 |
| PAX3 (isoform | 1192 |
| Pax3E) | |
| ALK | 1193 |
| Androgen receptor | 1194 |
| (isoform 1) | |
| Androgen receptor | 1195 |
| (isoform 2) | |
| Androgen receptor | 1196 |
| (isoform 3) | |
| Androgen receptor | 1197 |
| (isoform 4) | |
| Cyclin B1 (isoform | 1198 |
| 1) | |
| Cyclin B1 (isoform | 1199 |
| 2) | |
| MYCN | 1200 |
| RhoC | 1201 |
| TRP-2 | 1202 |
| TRP-2 | 1203 |
| TRP-2 | 1204 |
| PSCA | 1205 |
| MAGE A1 | 1206 |
| CYP1B1 | 1207 |
| BORIS (isoform 1) | 1208 |
| BORIS (isoform 2) | 1209 |
| BORIS (isoform 3) | 1210 |
| BORIS (isoform 4) | 1211 |
| BORIS (isoform 5) | 1212 |
| BORIS (isoform 6) | 1213 |
| BORIS (isoform 7) | 1214 |
| BORIS (isoform 8) | 1215 |
| BORIS (isoform 9) | 1216 |
| BORIS (isoform | 1217 |
| 10) | |
| BORIS (isoform | 1218 |
| 11) | |
| ETV6-AML | 1219 |
| NY-BR-1 | 1220 |
| RGS5 (isoform 1) | 1221 |
| RGS5 (isoform 2) | 1222 |
| RGS5 (isoform 3) | 1223 |
| SART3 (isoform 1) | 1224 |
| SART3 (isoform 2) | 1225 |
| SART3 (isoform 3) | 1226 |
| SART3 (isoform 4) | 1227 |
| Carbonic anhydrase | 1228 |
| IX | |
| PAX5 (isoform 1) | 1229 |
| PAX5 (isoform 2) | 1230 |
| PAX5 (isoform 3) | 1231 |
| PAX5 (isoform 4) | 1232 |
| PAX5 (isoform 5) | 1233 |
| PAX5 (isoform 6) | 1234 |
| PAX5 (isoform 7) | 1235 |
| PAX5 (isoform 8) | 1236 |
| PAX5 (isoform 9) | 1237 |
| PAX5 (isoform 10) | 1238 |
| PAX5 (isoform 11) | 1239 |
| OY-TES1 | 1240 |
| Sperm protein 17 | 1241 |
| LCK (isoform | 1242 |
| long) | |
| LCK (isoform | 1243 |
| short) | |
| LCK (isoform 3) | 1244 |
| HMWMAA | 1245 |
| AKAP-4 (isoform | 1246 |
| 1) | |
| AKAP-4 (isoform | 1247 |
| 2) | |
| SSX2 (isoform 1) | 1248 |
| SSX2 (isoform 2) | 1249 |
| XAGE 1 (isoform | 1250 |
| B) | |
| XAGE 1 (isoform | 1251 |
| D) | |
| Legumain (isoform | 1252 |
| 1) | |
| Legumain (isoform | 1253 |
| 2) | |
| Legumain (isoform | 1254 |
| 3) | |
| VEGFR2 (isoform | 1255 |
| 1) | |
| VEGFR2 (isoform | 1256 |
| 2) | |
| VEGFR2 (isoform | 1257 |
| 3) | |
| MAD-CT-1 | 1258 |
| (isoform 1) | |
| MAD-CT-1 | 1259 |
| (isoform 2) | |
| PDGFR-B (isoform | 1260 |
| 1) | |
| PDGFR-B (isoform | 1261 |
| 2) | |
| ROR2 | 1262 |
| TMPRSS3 | 1263 |
| (isoform A) | |
| TMPRSS3 | 1264 |
| (isoform B) | |
| TMPRSS3 | 1265 |
| (isoform D) | |
| TMPRSS3 | 1266 |
| (isoform T) | |
| TMPRSS3 | 1267 |
| (isoform E) | |
| TMPRSS3 | 1268 |
| (isoform 6) | |
| TMPRSS4 | 1269 |
| (isoform 1) | |
| TMPRSS4 | 1270 |
| (isoform 2) | |
| TMPRSS4 | 1271 |
| (isoform 3) | |
| TMPRSS4 | 1272 |
| (isoform 4) | |
| TMEM238 | 1273 |
| C1ORF186 | 1274 |
| Fos-related antigen | 1275 |
| 1 (isoform 1) | |
| Fos-related antigen | 1276 |
| 1 (isoform 2) | |
| VEGFR1 (isoform | 1277 |
| 1) | |
| VEGFR1 (isoform | 1278 |
| 2) | |
| VEGFR1 (isoform | 1279 |
| 3) | |
| VEGFR1 (isoform | 1280 |
| 4) | |
| VEGFR1 (isoform | 1281 |
| 5) | |
| VEGFR1 (isoform | 1282 |
| 6) | |
| VEGFR1 (isoform | 1283 |
| 7) | |
| VEGFR1 (isoform | 1284 |
| 8) | |
| Endoglin (isoform | 1285 |
| long) | |
| Endoglin (isoform | 1286 |
| short) | |
| VISTA | 1287 |
| MAGE C2 | 1288 |
| MAGE A4 | 1289 |
| GAGE 2B/C | 1290 |
| GAGE 7 | 1291 |
| GAGE 12B/C/D/E | 1292 |
| GAGE 2D | 1293 |
| GAGE 1 | 1294 |
| GAGE 5 | 1295 |
| GAGE 12I | 1296 |
| GAGE 12F | 1297 |
| GAGE 13 | 1298 |
| GAGE 12J | 1299 |
| GAGE 2A | 1300 |
| GAGE 6 | 1301 |
| GAGE 10 | 1302 |
| GAGE 4 | 1303 |
| GAGE 12G | 1304 |
| GAGE 12H | 1305 |
| GAGE 2E | 1306 |
| TRAIL1 (isoform | 1307 |
| long) | |
| TRAIL (isoform 3) | 1308 |
| TRAIL (isoform | 1309 |
| short) | |
| PAGE4 | 1310 |

In some embodiments, an amino acid sequence of the tumor antigen has at least 80% sequence identity with the amino acid sequence of a tumor antigen selected from the group consisting of HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, Her-2/neu, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, C1orf186, Fos-related antigen 1, VEGFR1, endoglin, PD-L1, VTCN1 (B7-H4), VISTA, or a fragment thereof, and a fragment thereof. In some embodiments, an amino acid sequence of the tumor antigen has at least 80% sequence identity with the amino acid sequence of a tumor antigen selected from TABLE 1.

In some embodiments, an amino acid sequence of the tumor antigen has at least 80% sequence identity with the amino acid sequence of a tumor antigen selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15, but not HER2 when the second binding domain specifically binds to CD40.

A binding domain of a conjugate or antibody construct can be selected from any domain that binds to an antigen including, but not limited to, from a monoclonal antibody, a polyclonal antibody, a recombinant antibody, or a functional fragment thereof, for example, a heavy chain variable domain (VH) and a light chain variable domain (VL), or from a non-antibody scaffold, such as a DARPin, an affimer, an avimer, a knottin, a monobody, lipocalin, an anticalin, ‘T-body’, an affibody, a peptibody, an affinity clamp, an ectodomain, a receptor ectodomain, a receptor, a cytokine, a ligand, an immunocytokine, a centryin, a T-cell receptor, or a recombinant T-cell receptor. The antigen binding domain of a conjugate or antibody construct can be at least 80%, at least 85%, at least 90%, at least 95%, or at least 99% homologous to an antigen binding domain selected from, but not limited to, a monoclonal antibody, a polyclonal antibody, a recombinant antibody, or a functional fragment thereof, for example, a heavy chain variable domain (VH) and a light chain variable domain (VL), or a DARPin, an affimer, an avimer, a knottin, a monobody, a lipocalin, an anticalin, ‘T-body’, an affibody, a peptibody, an affinity clamp, an ectodomain, a receptor ectodomain, a receptor, a cytokine, a ligand, an immunocytokine, a centryin, a T-cell receptor, or a recombinant T-cell receptor.

A binding domain of a conjugate or antibody construct, for example an antigen binding domain from a monoclonal antibody, can comprise a light chain and a heavy chain. In one aspect, the monoclonal antibody binds to an antigen present on the surface of an immune cell (immune cell antigen) and comprises the light chain of an anti-immune cell antigen antibody and the heavy chain of an anti-immune cell antigen antibody, which bind to an immune cell antigen. In another aspect, the monoclonal antibody binds to an antigen present on the surface of an antigen presenting cell (APC antigen) and comprises the light chain of an anti-APC antigen antibody and the heavy chain of an anti-APC antigen antibody, which bind to an APC antigen. In another aspect, the monoclonal antibody binds to CD40 and comprises the light chain of an anti-CD40 antibody and the heavy chain of an anti-CD40 antibody, which bind to a CD40 antigen. In another aspect, the monoclonal antibody binds to a tumor antigen comprises the light chain of a tumor antigen antibody and the heavy chain of a tumor antigen antibody, which bind to the tumor antigen.

A conjugate or antibody construct can comprise an antibody. An antibody molecule can consist of two identical light protein chains (light chains) and two identical heavy protein chains (heavy chains), all held together covalently by precisely located disulfide linkages. The N-terminal regions of the light and heavy chains together can form the antigen recognition site of each antibody. Structurally, various functions of an antibody can be confined to discrete protein domains (i.e., regions). The sites that can recognize and can bind to antigen consist of three complementarity determining regions (CDRs) that can lie within the variable heavy chain regions and variable light chain regions at the N-terminal ends of the two heavy and two light chains. The constant domains can provide the general framework of the antibody and may not be involved directly in binding the antibody to an antigen, but can be involved in various effector functions, such as participation of the antibody in antibody-dependent cellular cytotoxicity (ADCC).

The domains of natural light chain variable regions and heavy chain variable regions can have the same general structures, and each domain can comprise four framework regions, whose sequences can be somewhat conserved, connected by three hyper-variable regions or CDRs. The four framework regions can largely adopt a β-sheet conformation and the CDRs can form loops connecting, and in some aspects forming part of, the β-sheet structure. The CDRs in each chain can be held in close proximity by the framework regions and, with the CDRs from the other chain, can contribute to the formation of the antigen binding site.

An antibody of a conjugate or antibody construct can comprise an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, δ, ε, γ, and μ, respectively. The light chains can be one of either kappa or κ and lambda or λ, based on the amino acid sequences of the constant domains. The Fc region can comprise an Fc domain. An Fc receptor can bind to an Fc domain. A conjugate can also comprise any fragment or recombinant form thereof, including but not limited to an scFv, Fab, variable Fc fragment, domain antibody, and any other fragment thereof that can specifically bind to an antigen.

An antibody can comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. Examples of antibody binding domains can include, but are not limited to, Fab, variable Fv fragment and other fragments, combinations of fragments or types of fragments known or knowable to one of ordinary skill in the art.

A conjugate or antibody construct can comprise an antigen binding domain of an antibody. An antigen binding domain of an antibody can comprise one or more light chain (LC) CDRs (LCDRs) and one or more heavy chain (HC) CDRs (HCDRs), one or more LCDRs or one or more HCDRs. For example, an antibody binding domain of an antibody can comprise one or more of the following: a light chain complementary determining region 1 (LCDR1), a light chain complementary determining region 2 (LCDR2), or a light chain complementary determining region 3 (LCDR3). For another example, an antibody binding domain can comprise one or more of the following: a heavy chain complementary determining region 1 (HCDR1), a heavy chain complementary determining region 2 (HCDR2), or a heavy chain complementary determining region 3 (HCDR3). In some embodiments an antibody binding domain comprises all of the following: a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). Unless stated otherwise, the CDRs described herein can be defined according to the IMGT (the international ImMunoGeneTics information system). An antigen binding domain can comprise only the heavy chain of an antibody (e.g., does not include any other portion of the antibody). An antigen binding domain can comprise only the variable domain of the heavy chain of an antibody. Alternatively, an antigen binding domain can comprise only the light chain of an antibody. An antigen binding domain can comprise only the variable light chain of an antibody.

A conjugate or antibody construct can comprise an antibody fragment. An antibody fragment can include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; and (iii) a Fv fragment consisting of the VL and VH domains of a single arm of an antibody. Although the two domains of the Fv fragment, VL and VH, can be coded for by separate genes, they can be linked by a synthetic linker to be made as a single protein chain in which the VL and VH regions pair to form monovalent molecules.

F(ab′)₂ and Fab′ moieties can be produced, for example, recombinantly or by treating immunoglobulin (monoclonal antibody) with a protease such as pepsin and papain, and can include an antibody fragment generated by digesting immunoglobulin near the disulfide bonds existing between the hinge regions in each of the two H chains. The Fab fragment can also contain the constant domain of the light chain and the first constant domain (CH1) of the heavy chain. Fab′ fragments can differ from Fab fragments by the addition of a few residues at the carboxyl terminus of the heavy chain CH1 domain including one or more cysteine(s) from the antibody hinge region.

An Fv can be the minimum antibody fragment which contains a complete antigen-recognition and antigen-binding site. This region can consist of a dimer of one heavy chain and one light chain variable domain in tight, non-covalent association. In this configuration, the three CDRs of each variable domain can interact to define an antigen-binding site on the surface of the VH-VL dimer. A single variable domain (or half of an Fv comprising only three CDRs specific for an antigen) can recognize and bind to antigen, although at a lower affinity than the entire binding site.

An antibody used herein can be “humanized.” Humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which can contain minimal sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody can also comprise at least a portion of an immunoglobulin constant region (Fc), typically that of a human immunoglobulin consensus sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins that do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be a derivatized antibody. For example, derivatized antibodies can be modified by glycosylation, acetylation, pegylation, phosphorylation, amidation, derivatization by known protecting/blocking groups, proteolytic cleavage, or the like.

An antibody described herein can have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced or increased binding to an Fc receptor (FcR). FcR binding can be reduced or increased by, for example, mutating the immunoglobulin constant region segment of the antibody at particular regions necessary for FcR interactions.

An antibody described herein can be modified to acquire or improve at least one constant region-mediated biological effector function relative to an unmodified antibody, e.g., to enhance FcγR interactions. For example, an antibody with a constant region that binds FcγRIIA, FcγRIIB and/or FcγRIIIA with greater affinity than the corresponding wild type constant region can be produced according to the methods described herein.

An antibody described herein can bind to tumor cells, such as an antibody against a cell surface receptor or a tumor antigen.

A conjugate or antibody construct can comprise a first binding domain. A conjugate or antibody construct can comprise a first binding domain that specifically binds to an antigen. A conjugate or antibody construct can comprise a first binding domain that specifically binds to a tumor antigen. A first binding domain can specifically bind to a tumor antigen, wherein the tumor antigen has an amino acid sequence that comprises at least 80% homology to an amino acid sequence of an antigen selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, and a fragment thereof. A first binding domain can specifically bind to a tumor antigen, wherein the tumor antigen has an amino acid sequence that comprises at least 80% homology to an amino acid sequence of an antigen selected from the group consisting of EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4) and STRA6 and a fragment thereof. A first binding domain can specifically bind to a tumor antigen, wherein the tumor antigen has an amino acid sequence that comprises at least 80% homology to an amino acid sequence of an antigen selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4) and STRA6 and a fragment thereof. A conjugate can comprise a first binding domain that specifically binds to a tumor antigen on a tumor cell, to an immune cell such as an antigen presenting cell, to an immune cell other than an antigen presenting cell or to an antigen presenting cell.

A conjugate or antibody construct can comprise a first binding domain that specifically binds to a tumor antigen. A conjugate or antibody construct can comprise a first binding domain comprising one or more CDRs. A first binding domain can comprise at least 80% sequence identity to any sequence in TABLE 3. A first binding domain can comprise at least 80% sequence identity to any sequence in TABLE 3 or TABLE 4. A conjugate can comprise a first binding domain that binds to a tumor antigen, wherein the first binding domain comprises at least 80% sequence identity to: a) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 18, LCDR2 comprising an amino acid sequence of SEQ ID NO: 19, and LCDR3 comprising an amino acid sequence of SEQ ID NO: 20; b) HCDR1 comprising an amino acid sequence of SEQ ID NO: 26, HCDR2 comprising an amino acid sequence of SEQ ID NO: 27, HCDR3 comprising an amino acid sequence of SEQ ID NO: 28, LCDR1 comprising an amino acid sequence of SEQ ID NO: 31, LCDR2 comprising an amino acid sequence of SEQ ID NO: 32, and LCDR3 comprising an amino acid sequence of SEQ ID NO: 33; c) HCDR1 comprising an amino acid sequence of SEQ ID NO: 39, HCDR2 comprising an amino acid sequence of SEQ ID NO: 40, HCDR3 comprising an amino acid sequence of SEQ ID NO: 41, LCDR1 comprising an amino acid sequence of SEQ ID NO: 44, LCDR2 comprising an amino acid sequence of SEQ ID NO: 45, and LCDR3 comprising an amino acid sequence of SEQ ID NO: 46; d) HCDR1 comprising an amino acid sequence of SEQ ID NO: 52, HCDR2 comprising an amino acid sequence of SEQ ID NO: 53, HCDR3 comprising an amino acid sequence of SEQ ID NO: 54, LCDR1 comprising an amino acid sequence of SEQ ID NO: 57, LCDR2 comprising an amino acid sequence of SEQ ID NO: 58, and LCDR3 comprising an amino acid sequence of SEQ ID NO: 59; e) HCDR1 comprising an amino acid sequence of SEQ ID NO: 65, HCDR2 comprising an amino acid sequence of SEQ ID NO: 66, HCDR3 comprising an amino acid sequence of SEQ ID NO: 67, LCDR1 comprising an amino acid sequence of SEQ ID NO: 70, LCDR2 comprising an amino acid sequence of SEQ ID NO: 71, and LCDR3 comprising an amino acid sequence of SEQ ID NO: 72; f) HCDR1 comprising an amino acid sequence of SEQ ID NO: 78, HCDR2 comprising an amino acid sequence of SEQ ID NO: 79, HCDR3 comprising an amino acid sequence of SEQ ID NO: 80, LCDR1 comprising an amino acid sequence of SEQ ID NO: 83, LCDR2 comprising an amino acid sequence of SEQ ID NO: 84, and LCDR3 comprising an amino acid sequence of SEQ ID NO: 85; g) HCDR1 comprising an amino acid sequence of SEQ ID NO: 91, HCDR2 comprising an amino acid sequence of SEQ ID NO: 92, HCDR3 comprising an amino acid sequence of SEQ ID NO: 93, LCDR1 comprising an amino acid sequence of SEQ ID NO: 96, LCDR2 comprising an amino acid sequence of SEQ ID NO: 97, and LCDR3 comprising an amino acid sequence of SEQ ID NO: 98; h) HCDR1 comprising an amino acid sequence of SEQ ID NO: 104, HCDR2 comprising an amino acid sequence of SEQ ID NO: 105, HCDR3 comprising an amino acid sequence of SEQ ID NO: 106, LCDR1 comprising an amino acid sequence of SEQ ID NO: 109, LCDR2 comprising an amino acid sequence of SEQ ID NO: 110, and LCDR3 comprising an amino acid sequence of SEQ ID NO: 111; i) HCDR1 comprising an amino acid sequence of SEQ ID NO: 117, HCDR2 comprising an amino acid sequence of SEQ ID NO: 118, HCDR3 comprising an amino acid sequence of SEQ ID NO: 119, LCDR1 comprising an amino acid sequence of SEQ ID NO: 122, LCDR2 comprising an amino acid sequence of SEQ ID NO: 123, and LCDR3 comprising an amino acid sequence of SEQ ID NO: 124; j) HCDR1 comprising an amino acid sequence of SEQ ID NO: 130, HCDR2 comprising an amino acid sequence of SEQ ID NO: 131, HCDR3 comprising an amino acid sequence of SEQ ID NO: 132, LCDR1 comprising an amino acid sequence of SEQ ID NO: 135, LCDR2 comprising an amino acid sequence of SEQ ID NO: 136, and LCDR3 comprising an amino acid sequence of SEQ ID NO: 137; k) HCDR1 comprising an amino acid sequence of SEQ ID NO: 143, HCDR2 comprising an amino acid sequence of SEQ ID NO: 144, HCDR3 comprising an amino acid sequence of SEQ ID NO: 145, LCDR1 comprising an amino acid sequence of SEQ ID NO: 148, LCDR2 comprising an amino acid sequence of SEQ ID NO: 149, and LCDR3 comprising an amino acid sequence of SEQ ID NO: 150; 1) HCDR1 comprising an amino acid sequence of SEQ ID NO: 156, HCDR2 comprising an amino acid sequence of SEQ ID NO: 157, HCDR3 comprising an amino acid sequence of SEQ ID NO: 158, LCDR1 comprising an amino acid sequence of SEQ ID NO: 161, LCDR2 comprising an amino acid sequence of SEQ ID NO: 162, and LCDR3 comprising an amino acid sequence of SEQ ID NO: 163; m) HCDR1 comprising an amino acid sequence of SEQ ID NO: 169, HCDR2 comprising an amino acid sequence of SEQ ID NO: 170, HCDR3 comprising an amino acid sequence of SEQ ID NO: 171, LCDR1 comprising an amino acid sequence of SEQ ID NO: 174, LCDR2 comprising an amino acid sequence of SEQ ID NO: 175, and LCDR3 comprising an amino acid sequence of SEQ ID NO: 176; n) HCDR1 comprising an amino acid sequence of SEQ ID NO: 182, HCDR2 comprising an amino acid sequence of SEQ ID NO: 183, HCDR3 comprising an amino acid sequence of SEQ ID NO: 184, LCDR1 comprising an amino acid sequence of SEQ ID NO: 187, LCDR2 comprising an amino acid sequence of SEQ ID NO: 188, and LCDR3 comprising an amino acid sequence of SEQ ID NO: 189; o) HCDR1 comprising an amino acid sequence of SEQ ID NO: 195, HCDR2 comprising an amino acid sequence of SEQ ID NO: 196, HCDR3 comprising an amino acid sequence of SEQ ID NO: 197, LCDR1 comprising an amino acid sequence of SEQ ID NO: 200, LCDR2 comprising an amino acid sequence of SEQ ID NO: 201, and LCDR3 comprising an amino acid sequence of SEQ ID NO: 202; p) HCDR1 comprising an amino acid sequence of SEQ ID NO: 208, HCDR2 comprising an amino acid sequence of SEQ ID NO: 209, HCDR3 comprising an amino acid sequence of SEQ ID NO: 210, LCDR1 comprising an amino acid sequence of SEQ ID NO: 213, LCDR2 comprising an amino acid sequence of SEQ ID NO: 214, and LCDR3 comprising an amino acid sequence of SEQ ID NO: 215; q) HCDR1 comprising an amino acid sequence of SEQ ID NO: 805, HCDR2 comprising an amino acid sequence of SEQ ID NO: 806, HCDR3 comprising an amino acid sequence of SEQ ID NO: 807, LCDR1 comprising an amino acid sequence of SEQ ID NO: 808, LCDR2 comprising an amino acid sequence of SEQ ID NO: 809, and LCDR3 comprising an amino acid sequence of SEQ ID NO: 810; r) HCDR1 comprising an amino acid sequence of SEQ ID NO: 823, HCDR2 comprising an amino acid sequence of SEQ ID NO: 824, HCDR3 comprising an amino acid sequence of SEQ ID NO: 825, LCDR1 comprising an amino acid sequence of SEQ ID NO: 826, LCDR2 comprising an amino acid sequence of SEQ ID NO: 827, and LCDR3 comprising an amino acid sequence of SEQ ID NO: 828; s) HCDR1 comprising an amino acid sequence of SEQ ID NO: 221, HCDR2 comprising an amino acid sequence of SEQ ID NO: 222, HCDR3 comprising an amino acid sequence of SEQ ID NO: 223, LCDR1 comprising an amino acid sequence of SEQ ID NO: 226, LCDR2 comprising an amino acid sequence of SEQ ID NO: 227, and LCDR3 comprising an amino acid sequence of SEQ ID NO: 228; t) HCDR1 comprising an amino acid sequence of SEQ ID NO: 260, HCDR2 comprising an amino acid sequence of SEQ ID NO: 261, HCDR3 comprising an amino acid sequence of SEQ ID NO: 262, LCDR1 comprising an amino acid sequence of SEQ ID NO: 265, LCDR2 comprising an amino acid sequence of SEQ ID NO: 266, and LCDR3 comprising an amino acid sequence of SEQ ID NO: 267; u) HCDR1 comprising an amino acid sequence of SEQ ID NO: 273, HCDR2 comprising an amino acid sequence of SEQ ID NO: 274, HCDR3 comprising an amino acid sequence of SEQ ID NO: 275, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; v) HCDR1 comprising an amino acid sequence of SEQ ID NO: 286, HCDR2 comprising an amino acid sequence of SEQ ID NO: 287, HCDR3 comprising an amino acid sequence of SEQ ID NO: 288, LCDR1 comprising an amino acid sequence of SEQ ID NO: 291, LCDR2 comprising an amino acid sequence of SEQ ID NO: 292, and LCDR3 comprising an amino acid sequence of SEQ ID NO: 293; w) HCDR1 comprising an amino acid sequence of SEQ ID NO: 299, HCDR2 comprising an amino acid sequence of SEQ ID NO: 300, HCDR3 comprising an amino acid sequence of SEQ ID NO: 301, LCDR1 comprising an amino acid sequence of SEQ ID NO: 304, LCDR2 comprising an amino acid sequence of SEQ ID NO: 305, and LCDR3 comprising an amino acid sequence of SEQ ID NO: 306; x) HCDR1 comprising an amino acid sequence of SEQ ID NO: 312, HCDR2 comprising an amino acid sequence of SEQ ID NO: 313, HCDR3 comprising an amino acid sequence of SEQ ID NO: 314, LCDR1 comprising an amino acid sequence of SEQ ID NO: 317, LCDR2 comprising an amino acid sequence of SEQ ID NO: 318, and LCDR3 comprising an amino acid sequence of SEQ ID NO: 319; y) HCDR1 comprising an amino acid sequence of SEQ ID NO: 325, HCDR2 comprising an amino acid sequence of SEQ ID NO: 326, HCDR3 comprising an amino acid sequence of SEQ ID NO: 327, LCDR1 comprising an amino acid sequence of SEQ ID NO: 330, LCDR2 comprising an amino acid sequence of SEQ ID NO: 331, and LCDR3 comprising an amino acid sequence of SEQ ID NO: 332; z) HCDR1 comprising an amino acid sequence of SEQ ID NO: 338, HCDR2 comprising an amino acid sequence of SEQ ID NO: 339, HCDR3 comprising an amino acid sequence of SEQ ID NO: 340, LCDR1 comprising an amino acid sequence of SEQ ID NO: 343, LCDR2 comprising an amino acid sequence of SEQ ID NO: 344, and LCDR3 comprising an amino acid sequence of SEQ ID NO: 345; aa) HCDR1 comprising an amino acid sequence of SEQ ID NO: 351, HCDR2 comprising an amino acid sequence of SEQ ID NO: 352, HCDR3 comprising an amino acid sequence of SEQ ID NO: 353, LCDR1 comprising an amino acid sequence of SEQ ID NO: 356, LCDR2 comprising an amino acid sequence of SEQ ID NO: 357, and LCDR3 comprising an amino acid sequence of SEQ ID NO: 358; bb) HCDR1 comprising an amino acid sequence of SEQ ID NO: 364, HCDR2 comprising an amino acid sequence of SEQ ID NO: 365, HCDR3 comprising an amino acid sequence of SEQ ID NO: 366, LCDR1 comprising an amino acid sequence of SEQ ID NO: 369, LCDR2 comprising an amino acid sequence of SEQ ID NO: 370, and LCDR3 comprising an amino acid sequence of SEQ ID NO: 371; cc) HCDR1 comprising an amino acid sequence of SEQ ID NO: 377, HCDR2 comprising an amino acid sequence of SEQ ID NO: 378, HCDR3 comprising an amino acid sequence of SEQ ID NO: 379, LCDR1 comprising an amino acid sequence of SEQ ID NO: 382, LCDR2 comprising an amino acid sequence of SEQ ID NO: 383, and LCDR3 comprising an amino acid sequence of SEQ ID NO: 384; dd) HCDR1 comprising an amino acid sequence of SEQ ID NO: 390, HCDR2 comprising an amino acid sequence of SEQ ID NO: 391, HCDR3 comprising an amino acid sequence of SEQ ID NO: 392, LCDR1 comprising an amino acid sequence of SEQ ID NO: 395, LCDR2 comprising an amino acid sequence of SEQ ID NO: 396, and LCDR3 comprising an amino acid sequence of SEQ ID NO: 397; ee) HCDR1 comprising an amino acid sequence of SEQ ID NO: 403, HCDR2 comprising an amino acid sequence of SEQ ID NO: 404, HCDR3 comprising an amino acid sequence of SEQ ID NO: 405, LCDR1 comprising an amino acid sequence of SEQ ID NO: 408, LCDR2 comprising an amino acid sequence of SEQ ID NO: 409, and LCDR3 comprising an amino acid sequence of SEQ ID NO: 410; ff) HCDR1 comprising an amino acid sequence of SEQ ID NO: 416, HCDR2 comprising an amino acid sequence of SEQ ID NO: 417, HCDR3 comprising an amino acid sequence of SEQ ID NO: 418, LCDR1 comprising an amino acid sequence of SEQ ID NO: 421, LCDR2 comprising an amino acid sequence of SEQ ID NO: 422, and LCDR3 comprising an amino acid sequence of SEQ ID NO: 423; gg) HCDR1 comprising an amino acid sequence of SEQ ID NO: 429, HCDR2 comprising an amino acid sequence of SEQ ID NO: 430, HCDR3 comprising an amino acid sequence of SEQ ID NO: 431, LCDR1 comprising an amino acid sequence of SEQ ID NO: 434, LCDR2 comprising an amino acid sequence of SEQ ID NO: 435, and LCDR3 comprising an amino acid sequence of SEQ ID NO: 436; hh) HCDR1 comprising an amino acid sequence of SEQ ID NO: 442, HCDR2 comprising an amino acid sequence of SEQ ID NO: 443, HCDR3 comprising an amino acid sequence of SEQ ID NO: 444, LCDR1 comprising an amino acid sequence of SEQ ID NO: 447, LCDR2 comprising an amino acid sequence of SEQ ID NO: 448, and LCDR3 comprising an amino acid sequence of SEQ ID NO: 449; ii) HCDR1 comprising an amino acid sequence of SEQ ID NO: 455, HCDR2 comprising an amino acid sequence of SEQ ID NO: 456, HCDR3 comprising an amino acid sequence of SEQ ID NO: 457, LCDR1 comprising an amino acid sequence of SEQ ID NO: 460, LCDR2 comprising an amino acid sequence of SEQ ID NO: 461, and LCDR3 comprising an amino acid sequence of SEQ ID NO: 462; jj) HCDR1 comprising an amino acid sequence of SEQ ID NO: 468, HCDR2 comprising an amino acid sequence of SEQ ID NO: 469, HCDR3 comprising an amino acid sequence of SEQ ID NO: 470, LCDR1 comprising an amino acid sequence of SEQ ID NO: 473, LCDR2 comprising an amino acid sequence of SEQ ID NO: 474, and LCDR3 comprising an amino acid sequence of SEQ ID NO: 475; kk) HCDR1 comprising an amino acid sequence of SEQ ID NO: 481, HCDR2 comprising an amino acid sequence of SEQ ID NO: 482, HCDR3 comprising an amino acid sequence of SEQ ID NO: 483, LCDR1 comprising an amino acid sequence of SEQ ID NO: 486, LCDR2 comprising an amino acid sequence of SEQ ID NO: 487, and LCDR3 comprising an amino acid sequence of SEQ ID NO: 488; ll) HCDR1 comprising an amino acid sequence of SEQ ID NO: 494, HCDR2 comprising an amino acid sequence of SEQ ID NO: 495, HCDR3 comprising an amino acid sequence of SEQ ID NO: 496, LCDR1 comprising an amino acid sequence of SEQ ID NO: 499, LCDR2 comprising an amino acid sequence of SEQ ID NO: 500, and LCDR3 comprising an amino acid sequence of SEQ ID NO: 501; or mm) HCDR1 comprising an amino acid sequence of SEQ ID NO: 673, HCDR2 comprising an amino acid sequence of SEQ ID NO: 674, HCDR3 comprising an amino acid sequence of SEQ ID NO: 675, LCDR1 comprising an amino acid sequence of SEQ ID NO: 676, LCDR2 comprising an amino acid sequence of SEQ ID NO: 677, and LCDR3 comprising an amino acid sequence of SEQ ID NO: 678.

A conjugate or antibody construct can comprise a first binding domain that specifically binds to a tumor antigen. A conjugate can comprise a first binding domain comprising one or more variable domains. A conjugate or antibody construct can comprise a first binding domain comprising a light chain variable domain (VL domain). A first binding domain can comprise a VL sequence in TABLE 5. A first binding domain can comprise at least 80% sequence identity to a VL sequence in TABLE 5. A conjugate or antibody construct can comprise a first binding domain comprising a heavy chain variable domain (VH domain). A first binding domain can comprise VH sequence in TABLE 5. A first binding domain can comprise at least 80% sequence identity to any VH sequence in TABLE 5. A first binding domain can comprise at least 80% sequence identity to a sequence in TABLE 5. A conjugate or antibody construct can comprise a first binding domain comprising a light chain variable domain (VL domain). A first binding domain can comprise a VL sequence in TABLE 5 or TABLE 6. A first binding domain can comprise at least 80% sequence identity to a VL sequence in TABLE 5 or TABLE 6. A conjugate or antibody construct can comprise a first binding domain comprising a heavy chain variable domain (VH domain). A first binding domain can comprise VH sequence in TABLE 5 or TABLE 6. A first binding domain can comprise at least 80% sequence identity to any VH sequence in TABLE 5 or TABLE 6. A first binding domain can comprise at least 80% sequence identity to a sequence in TABLE 5 or TABLE 6.

A conjugate or antibody construct can comprise a first binding domain that specifically binds to a tumor antigen, wherein the first binding domain comprises: a) a VH sequence having at least 80% sequence identity to an amino acid sequence of SEQ ID NO: 12, and a VL sequence having at least 80% sequence identity to an amino acid sequence of SEQ ID NO: 17; b) a VH sequence having at least 80% sequence identity to an amino acid sequence of SEQ ID NO: 25, and a VL sequence having at least 80% sequence identity to an amino acid sequence of SEQ ID NO: 30; c) a VH sequence having at least 80% sequence identity to an amino acid sequence of SEQ ID NO: 38, and a VL sequence having at least 80% sequence identity to an amino acid sequence of SEQ ID NO: 43; d) a VH sequence having at least 80% sequence identity to an amino acid sequence of SEQ ID NO: 51, and a VL sequence having at least 80% sequence identity to an amino acid sequence of SEQ ID NO: 56; e) a VH sequence having at least 80% sequence identity to an amino acid sequence of SEQ ID NO: 64, and a VL sequence having at least 80% sequence identity to an amino acid sequence of SEQ ID NO: 69; f) a VH sequence having at least 80% sequence identity to an amino acid sequence of SEQ ID NO: 77, and a VL sequence having at least 80% sequence identity to an amino acid sequence of SEQ ID NO: 82; g) a VH sequence having at least 80% sequence identity to an amino acid sequence of SEQ ID NO: 90, and a VL sequence having at least 80% sequence identity to an amino acid sequence of SEQ ID NO: 95; h) a VH sequence having at least 80% sequence identity to an amino acid sequence of SEQ ID NO: 103, and a VL sequence having at least 80% sequence identity to an amino acid sequence of SEQ ID NO: 108; i) a VH sequence having at least 80% sequence identity to an amino acid sequence of SEQ ID NO: 116, and a VL sequence having at least 80% sequence identity to an amino acid sequence of SEQ ID NO: 121; j) a VH sequence having at least 80% sequence identity to an amino acid sequence of SEQ ID NO: 129, and a VL sequence having at least 80% sequence identity to an amino acid sequence of SEQ ID NO: 134; k) a VH sequence having at least 80% sequence identity to an amino acid sequence of SEQ ID NO: 142, and a VL sequence having at least 80% sequence identity to an amino acid sequence of SEQ ID NO: 147; 1) a VH sequence having at least 80% sequence identity to an amino acid sequence of SEQ ID NO: 155, and a VL sequence having at least 80% sequence identity to an amino acid sequence of SEQ ID NO: 160; m) a VH sequence having at least 80% sequence identity to an amino acid sequence of SEQ ID NO: 168, and a VL sequence having at least 80% sequence identity to an amino acid sequence of SEQ ID NO: 173; n) a VH sequence having at least 80% sequence identity to an amino acid sequence of SEQ ID NO: 181, and a VL sequence having at least 80% sequence identity to an amino acid sequence of SEQ ID NO: 186; o) a VH sequence having at least 80% sequence identity to an amino acid sequence of SEQ ID NO: 194, and a VL sequence having at least 80% sequence identity to an amino acid sequence of SEQ ID NO: 199; p) a VH sequence having at least 80% sequence identity to an amino acid sequence of SEQ ID NO: 207, and a VL sequence having at least 80% sequence identity to an amino acid sequence of SEQ ID NO: 212; q) a VH sequence having at least 80% sequence identity to an amino acid sequence of SEQ ID NO: 811, and a VL sequence having at least 80% sequence identity to an amino acid sequence of SEQ ID NO: 812; r) a VH sequence having at least 80% sequence identity to an amino acid sequence of SEQ ID NO: 829, and a VL sequence having at least 80% sequence identity to an amino acid sequence of SEQ ID NO: 830; s) a VH sequence having at least 80% sequence identity to an amino acid sequence of SEQ ID NO: 220, and a VL sequence having at least 80% sequence identity to an amino acid sequence of SEQ ID NO: 225; t) a VH sequence having at least 80% sequence identity to an amino acid sequence of SEQ ID NO: 259, and a VL sequence having at least 80% sequence identity to an amino acid sequence of SEQ ID NO: 264; u) a VH sequence having at least 80% sequence identity to an amino acid sequence of SEQ ID NO: 272, and a VL sequence having at least 80% sequence identity to an amino acid sequence of SEQ ID NO: 277; v) a VH sequence having at least 80% sequence identity to an amino acid sequence of SEQ ID NO: 285, and a VL sequence having at least 80% sequence identity to an amino acid sequence of SEQ ID NO: 290; w) a VH sequence having at least 80% sequence identity to an amino acid sequence of SEQ ID NO: 298, and a VL sequence having at least 80% sequence identity to an amino acid sequence of SEQ ID NO: 303; x) a VH sequence having at least 80% sequence identity to an amino acid sequence of SEQ ID NO: 311, and a VL sequence having at least 80% sequence identity to an amino acid sequence of SEQ ID NO: 316; y) a VH sequence having at least 80% sequence identity to an amino acid sequence of SEQ ID NO: 324, and a VL sequence having at least 80% sequence identity to an amino acid sequence of SEQ ID NO: 328; z) a VH sequence having at least 80% sequence identity to an amino acid sequence of SEQ ID NO: 337, and a VL sequence having at least 80% sequence identity to an amino acid sequence of SEQ ID NO: 342; aa) a VH sequence having at least 80% sequence identity to an amino acid sequence of SEQ ID NO: 350, and a VL sequence having at least 80% sequence identity to an amino acid sequence of SEQ ID NO: 355; bb) a VH sequence having at least 80% sequence identity to an amino acid sequence of SEQ ID NO: 363, and a VL sequence having at least 80% sequence identity to an amino acid sequence of SEQ ID NO: 368; cc) a VH sequence having at least 80% sequence identity to an amino acid sequence of SEQ ID NO: 376, and a VL sequence having at least 80% sequence identity to an amino acid sequence of SEQ ID NO: 381; dd) a VH sequence having at least 80% sequence identity to an amino acid sequence of SEQ ID NO: 389, and a VL sequence having at least 80% sequence identity to an amino acid sequence of SEQ ID NO: 394; ee) a VH sequence having at least 80% sequence identity to an amino acid sequence of SEQ ID NO: 402, and a VL sequence having at least 80% sequence identity to an amino acid sequence of SEQ ID NO: 407; ff) a VH sequence having at least 80% sequence identity to an amino acid sequence of SEQ ID NO: 415, and a VL sequence having at least 80% sequence identity to an amino acid sequence of SEQ ID NO: 420; gg) a VH sequence having at least 80% sequence identity to an amino acid sequence of SEQ ID NO: 428, and a VL sequence having at least 80% sequence identity to an amino acid sequence of SEQ ID NO: 433; hh) a VH sequence having at least 80% sequence identity to an amino acid sequence of SEQ ID NO: 441, and a VL sequence having at least 80% sequence identity to an amino acid sequence of SEQ ID NO: 446; ii) a VH sequence having at least 80% sequence identity to an amino acid sequence of SEQ ID NO: 454, and a VL sequence having at least 80% sequence identity to an amino acid sequence of SEQ ID NO: 459; jj) a VH sequence having at least 80% sequence identity to an amino acid sequence of SEQ ID NO: 467, and a VL sequence having at least 80% sequence identity to an amino acid sequence of SEQ ID NO: 472; kk) a VH sequence having at least 80% sequence identity to an amino acid sequence of SEQ ID NO: 480, and a VL sequence having at least 80% sequence identity to an amino acid sequence of SEQ ID NO: 485; ll) a VH sequence having at least 80% sequence identity to an amino acid sequence of SEQ ID NO: 493, and a VL sequence having at least 80% sequence identity to an amino acid sequence of SEQ ID NO: 498; or mm) a VH sequence having at least 80% sequence identity to an amino acid sequence of SEQ ID NO: 679, and a VL sequence having at least 80% sequence identity to an amino acid sequence of SEQ ID NO: 680.

A conjugate or antibody construct can comprise a first binding domain and an Fc domain, wherein the first binding domain and the Fc domain comprise an antibody. A first binding domain can bind to a tumor antigen. A conjugate or antibody construct can comprise an antibody light chain. A conjugate or antibody construct can comprise a light chain comprising a light chain sequence in TABLE 7. A conjugate or antibody construct can comprise a light chain comprising at least 80% sequence identity to a light chain sequence in TABLE 7. A conjugate or antibody construct can comprise an antibody heavy chain. A conjugate or antibody construct can comprise a heavy chain comprising a heavy chain sequence in TABLE 7. A conjugate or antibody construct can comprise a heavy chain comprising at least 80% sequence identity to any heavy chain sequence in TABLE 7 or TABLE 8. A conjugate or antibody construct can comprise at least 80% sequence identity to any sequence in TABLE 7. A conjugate or antibody construct can comprise a first binding domain and an Fc domain, wherein the first binding domain and the Fc domain comprise an antibody. A first binding domain can bind to a tumor antigen. A conjugate or antibody construct can comprise an antibody light chain. A conjugate or antibody construct can comprise a light chain comprising a light chain sequence in TABLE 7 or TABLE 8. A conjugate or antibody construct can comprise a light chain comprising at least 80% sequence identity to a light chain sequence in TABLE 7 or TABLE 8. A conjugate or antibody construct can comprise an antibody heavy chain. A conjugate or antibody construct can comprise a heavy chain comprising a heavy chain sequence in TABLE 7 or TABLE 8. A conjugate or antibody construct can comprise a heavy chain comprising at least 80% sequence identity to any heavy chain sequence in TABLE 7 or TABLE 8. A conjugate or antibody construct can comprise at least 80% sequence identity to any sequence in TABLE 7 or TABLE 8.

A conjugate or antibody construct can comprise an anti-tumor antibody, wherein the antibody comprises: a) a heavy chain sequence having at least 80% sequence identity to an amino acid sequence of SEQ ID NO: 11, and a light chain sequence having at least 80% sequence identity to an amino acid sequence of SEQ ID NO: 16; b) a heavy chain sequence having at least 80% sequence identity to an amino acid sequence of SEQ ID NO: 24, and a light chain sequence having at least 80% sequence identity to an amino acid sequence of SEQ ID NO: 29; c) a heavy chain sequence having at least 80% sequence identity to an amino acid sequence of SEQ ID NO: 37, and a light chain sequence having at least 80% sequence identity to an amino acid sequence of SEQ ID NO: 42; d) a heavy chain sequence having at least 80% sequence identity to an amino acid sequence of SEQ ID NO: 50, and a light chain sequence having at least 80% sequence identity to an amino acid sequence of SEQ ID NO: 55; e) a heavy chain sequence having at least 80% sequence identity to an amino acid sequence of SEQ ID NO: 63, and a light chain sequence having at least 80% sequence identity to an amino acid sequence of SEQ ID NO: 68; f) a heavy chain sequence having at least 80% sequence identity to an amino acid sequence of SEQ ID NO: 76, and a light chain sequence having at least 80% sequence identity to an amino acid sequence of SEQ ID NO: 81; g) a heavy chain sequence having at least 80% sequence identity to an amino acid sequence of SEQ ID NO: 89, and a light chain sequence having at least 80% sequence identity to an amino acid sequence of SEQ ID NO: 94; h) a heavy chain sequence having at least 80% sequence identity to an amino acid sequence of SEQ ID NO: 102, and a light chain sequence having at least 80% sequence identity to an amino acid sequence of SEQ ID NO: 107; i) a heavy chain sequence having at least 80% sequence identity to an amino acid sequence of SEQ ID NO: 115, and a light chain sequence having at least 80% sequence identity to an amino acid sequence of SEQ ID NO: 120; j) a heavy chain sequence having at least 80% sequence identity to an amino acid sequence of SEQ ID NO: 128, and a light chain sequence having at least 80% sequence identity to an amino acid sequence of SEQ ID NO: 133; k) a heavy chain sequence having at least 80% sequence identity to an amino acid sequence of SEQ ID NO: 141, and a light chain sequence having at least 80% sequence identity to an amino acid sequence of SEQ ID NO: 146; 1) a heavy chain sequence having at least 80% sequence identity to an amino acid sequence of SEQ ID NO: 154, and a light chain sequence having at least 80% sequence identity to an amino acid sequence of SEQ ID NO: 159; m) a heavy chain sequence having at least 80% sequence identity to an amino acid sequence of SEQ ID NO: 167, and a light chain sequence having at least 80% sequence identity to an amino acid sequence of SEQ ID NO: 172; n) a heavy chain sequence having at least 80% sequence identity to an amino acid sequence of SEQ ID NO: 180, and a light chain sequence having at least 80% sequence identity to an amino acid sequence of SEQ ID NO: 185; o) a heavy chain sequence having at least 80% sequence identity to an amino acid sequence of SEQ ID NO: 193, and a light chain sequence having at least 80% sequence identity to an amino acid sequence of SEQ ID NO: 198; p) a heavy chain sequence having at least 80% sequence identity to an amino acid sequence of SEQ ID NO: 206, and a light chain sequence having at least 80% sequence identity to an amino acid sequence of SEQ ID NO: 211; q) a heavy chain sequence having at least 80% sequence identity to an amino acid sequence of SEQ ID NO: 813, and a light chain sequence having at least 80% sequence identity to an amino acid sequence of SEQ ID NO: 814; r) a heavy chain sequence having at least 80% sequence identity to an amino acid sequence of SEQ ID NO: 831, and a light chain sequence having at least 80% sequence identity to an amino acid sequence of SEQ ID NO: 832; s) a heavy chain sequence having at least 80% sequence identity to an amino acid sequence of SEQ ID NO: 219, and a light chain sequence having at least 80% sequence identity to an amino acid sequence of SEQ ID NO: 224; t) a heavy chain sequence having at least 80% sequence identity to an amino acid sequence of SEQ ID NO: 258, and a light chain sequence having at least 80% sequence identity to an amino acid sequence of SEQ ID NO: 263; u) a heavy chain sequence having at least 80% sequence identity to an amino acid sequence of SEQ ID NO: 271, and a light chain sequence having at least 80% sequence identity to an amino acid sequence of SEQ ID NO: 276; v) a heavy chain sequence having at least 80% sequence identity to an amino acid sequence of SEQ ID NO: 284, and a light chain sequence having at least 80% sequence identity to an amino acid sequence of SEQ ID NO: 289; w) a heavy chain sequence having at least 80% sequence identity to an amino acid sequence of SEQ ID NO: 297, and a light chain sequence having at least 80% sequence identity to an amino acid sequence of SEQ ID NO: 302; x) a heavy chain sequence having at least 80% sequence identity to an amino acid sequence of SEQ ID NO: 310, and a light chain sequence having at least 80% sequence identity to an amino acid sequence of SEQ ID NO: 315; y) a heavy chain sequence having at least 80% sequence identity to an amino acid sequence of SEQ ID NO: 323, and a light chain sequence having at least 80% sequence identity to an amino acid sequence of SEQ ID NO: 328; z) a heavy chain sequence having at least 80% sequence identity to an amino acid sequence of SEQ ID NO: 336, and a light chain sequence having at least 80% sequence identity to an amino acid sequence of SEQ ID NO: 341; aa) a heavy chain sequence having at least 80% sequence identity to an amino acid sequence of SEQ ID NO: 349, and a light chain sequence having at least 80% sequence identity to an amino acid sequence of SEQ ID NO: 354; bb) a heavy chain sequence having at least 80% sequence identity to an amino acid sequence of SEQ ID NO: 362, and a light chain sequence having at least 80% sequence identity to an amino acid sequence of SEQ ID NO: 367; cc) a heavy chain sequence having at least 80% sequence identity to an amino acid sequence of SEQ ID NO: 375, and a light chain sequence having at least 80% sequence identity to an amino acid sequence of SEQ ID NO: 380; dd) a heavy chain sequence having at least 80% sequence identity to an amino acid sequence of SEQ ID NO: 388, and a light chain sequence having at least 80% sequence identity to an amino acid sequence of SEQ ID NO: 393; ee) a heavy chain sequence having at least 80% sequence identity to an amino acid sequence of SEQ ID NO: 401, and a light chain sequence having at least 80% sequence identity to an amino acid sequence of SEQ ID NO: 406; ff) a heavy chain sequence having at least 80% sequence identity to an amino acid sequence of SEQ ID NO: 414, and a light chain sequence having at least 80% sequence identity to an amino acid sequence of SEQ ID NO: 419; gg) a heavy chain sequence having at least 80% sequence identity to an amino acid sequence of SEQ ID NO: 427, and a light chain sequence having at least 80% sequence identity to an amino acid sequence of SEQ ID NO: 432; hh) a heavy chain sequence having at least 80% sequence identity to an amino acid sequence of SEQ ID NO: 440, and a light chain sequence having at least 80% sequence identity to an amino acid sequence of SEQ ID NO: 445; ii) a heavy chain sequence having at least 80% sequence identity to an amino acid sequence of SEQ ID NO: 453, and a light chain sequence having at least 80% sequence identity to an amino acid sequence of SEQ ID NO: 458; jj) a heavy chain sequence having at least 80% sequence identity to an amino acid sequence of SEQ ID NO: 466, and a light chain sequence having at least 80% sequence identity to an amino acid sequence of SEQ ID NO: 471; kk) a heavy chain sequence having at least 80% sequence identity to an amino acid sequence of SEQ ID NO: 479, and a light chain sequence having at least 80% sequence identity to an amino acid sequence of SEQ ID NO: 484; ll) a heavy chain sequence having at least 80% sequence identity to an amino acid sequence of SEQ ID NO: 492, and a light chain sequence having at least 80% sequence identity to an amino acid sequence of SEQ ID NO: 497; or mm) a heavy chain sequence having at least 80% sequence identity to an amino acid sequence of SEQ ID NO: 681, and a light chain sequence having at least 80% sequence identity to an amino acid sequence of SEQ ID NO: 682.

A conjugate or antibody construct can comprise a second binding domain. A conjugate or antibody construct can comprise a second binding domain that specifically binds to an antigen. A conjugate or antibody construct can comprise a second binding domain that specifically binds to a molecule on an immune cell. An immune cell can be a T cell, B cell, dendritic cell, macrophage, NK cell, or NKT cell. In some embodiments, an immune cell is a T cell, B cell, NK cell, or NKT cell. In some embodiments, an immune cell is an antigen presenting cell. A conjugate or antibody construct can comprise a second binding domain that specifically binds to a molecule on an immune cell such as an antigen presenting cell. An antigen presenting cell can be a dendritic cell or a macrophage. A second binding domain can specifically bind to a molecule on an immune cell, wherein the molecule comprises at least 80% homology to an amino acid sequence of a group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, CD32B, PD-L1, and CD47. A second binding domain can specifically bind to a molecule on an immune cell, wherein the molecule comprises at least 80% homology to an amino acid sequence of a group consisting of DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, and CD32B. A second binding domain can specifically bind to a molecule on an immune cell, wherein the molecule comprises at least 80% homology to an amino acid sequence of a group consisting of tumor necrosis factor receptor 2 (TNFR2) or triggering receptor expressed on myeloid cells 2 (TREM2). A second binding domain can specifically bind to a molecule on an antigen presenting cell, wherein the molecule comprises at least 80% homology to a group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD47, and CD32B. A second binding domain can specifically bind to a molecule on an antigen presenting cell, wherein the molecule comprises at least 80% homology to a group consisting of DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, and CD32B. TABLE 2 shows exemplary amino acid sequences of molecules on an immune cell to which a second binding domain can specifically bind.

TABLE 2
Exemplary amino acid sequences of molecules on an immune
cell to which a second binding domain can specifically bind

| Immune Cell Molecule | SEQ ID NO: |
|---|---|
| CD40 (isoform I) | 958 |
| CD40 (isoform II) | 959 |
| DEC-205 (isoform 4) | 960 |
| DEC-205 (isoform 2) | 961 |
| DEC-205 (isoform 5) | 962 |
| DEC-205 (isoform 3) | 963 |
| DEC-205 (isoform 1) | 964 |
| CD36 mannose | 965 |
| scavenger receptor 1 | |
| (isoform 1) | |
| CD36 mannose | 966 |
| scavenger receptor 1 | |
| (isoform 2) | |
| CLEC9A | 967 |
| DC-SIGN (isoform 1) | 968 |
| DC-SIGN (isoform 2) | 969 |
| DC-SIGN (isoform 3) | 970 |
| DC-SIGN (isoform 4) | 971 |
| DC-SIGN (isoform 5) | 972 |
| DC-SIGN (isoform 6) | 973 |
| DC-SIGN (isoform 7) | 974 |
| DC-SIGN (isoform 8) | 975 |
| DC-SIGN (isoform 9) | 976 |
| DC-SIGN (isoform 10) | 977 |
| DC-SIGN (isoform 11) | 978 |
| DC-SIGN (isoform 12) | 979 |
| CLEC12A (isoform 2) | 980 |
| CLEC12A (isoform 1) | 981 |
| CLEC12A (isoform 3) | 982 |
| CLEC12A (isoform 4) | 983 |
| CLEC12A (isoform 5) | 984 |
| BDCA-2 (isoform 1) | 985 |
| BDCA-2 (isoform 2) | 986 |
| OX40L (isoform 1) | 987 |
| OX40L (isoform 2) | 988 |
| 41BBL | 989 |
| CD204 (isoform I) | 990 |
| CD204 (isoform II) | 991 |
| CD204 (isoform III) | 992 |
| MARCO (isoform 1) | 993 |
| MARCO (isoform 2) | 994 |
| CLEC5A (isoform 1) | 995 |
| CLEC5A (isoform 2) | 996 |
| Dectin 1 (isoform 1) | 997 |
| Dectin 1 (isoform 2) | 998 |
| Dectin 1 (isoform 3) | 999 |
| Dectin 1 (isoform 4) | 1000 |
| Dectin 1 (isoform 5) | 1001 |
| Dectin 1 (isoform 6) | 1002 |
| Dectin 1 (isoform 7) | 1003 |
| Dectin 1 (isoform 8) | 1004 |
| Dectin 1 (isoform 9) | 1005 |
| Dectin 1 (isoform 10) | 1006 |
| Dectin 2 (isoform 1) | 1007 |
| Dectin 2 (isoform 2) | 1008 |
| CLEC10A (isoform 1) | 1009 |
| CLEC10A (isoform 2) | 1010 |
| CLEC10A (isoform 3) | 1011 |
| CD206 (isoform 1) | 1012 |
| CD206 (isoform 2) | 1013 |
| CD64 (isoform 1) | 1014 |
| CD64 (isoform 2) | 1015 |
| CD32A (isoform 1) | 1016 |
| CD32A (isoform 2) | 1017 |
| CD32A (isoform 3) | 1018 |
| CD16A | 1019 |
| HVEM (isoform 1) | 1020 |
| HVEM (isoform 2) | 1021 |
| CD32B (isoform IIB2) | 1022 |
| CD32B (isoform IIB3) | 1023 |
| CD32B (isoform 4) | 1024 |
| CD32B (isoform 5) | 1025 |
| PD-L1 (isoform 1) | 955 |
| PD-L1 (isoform 2) | 956 |
| PD-L1 (isoform 3) | 957 |
| CD47 (isoform OA3- | 1026 |
| 323) | |
| CD47 (isoform OA3- | 1027 |
| 293) | |
| CD47 (isoform OA3- | 1028 |
| 305) | |
| CD47 (isoform OA3- | 1029 |
| 312) | |

In some embodiments, an amino acid sequence of the antigen on the antigen presenting cell has at least 80% sequence identity with the amino acid sequence of an antigen selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, and CD47, but not CD40 when the first binding domain specifically binds to HER2.

In some embodiments, an amino acid sequence of the antigen on the antigen presenting cell has at least 80% sequence identity with the amino acid sequence of an antigen selected from TABLE 2. In some embodiments, the second binding domain is a CD40 agonist. In some embodiments, the first binding domain comprises a single chain variable fragment (scFv). In some embodiments, the second binding domain is a single chain variable fragment (scFv). In some embodiments, the second binding domain comprises a single chain variable fragment from an anti-CD40 antibody, an anti-DEC-205 antibody, an anti-CD36 mannose scavenger receptor 1 antibody, an anti-DC-SIGN antibody, an anti-CLEC9A antibody, an anti-CLEC12A antibody, an anti-BDCA-2 antibody, an anti-OX40L antibody, an anti-41BBL antibody, an anti-CD204 antibody, an anti-MARCO antibody, an anti-CLEC5A antibody, an anti-Dectin 1 antibody, an anti-Dectin 2 antibody, an anti-CLEC10A antibody, an anti-CD206 antibody, an anti-CD64 antibody, an anti-CD32A antibody, an anti-CD16A antibody, an anti-HVEM antibody, an anti-PD-L1, or an anti-CD32B antibody.

A conjugate or antibody construct can comprise an Fc domain. A conjugate or antibody construct can comprise a first binding domain, a second binding domain, and an Fc domain, wherein the first binding domain is attached to the Fc domain. A conjugate or antibody construct can comprise a first binding domain, a second binding domain, and an Fc domain, wherein the second binding domain is attached to the Fc domain. A first binding domain can be attached to an Fc domain as a fusion peptide. A second binding domain can be attached to an Fc domain as a fusion peptide. A first binding domain can be attached to an Fc domain via a linker. A second binding domain can be attached to an Fc domain via a linker.

A conjugate or antibody construct can comprise a second binding domain comprising one or more CDRs. A second binding domain can comprise a sequence or pair of sequences in TABLE 11. A second binding domain can comprise a sequence or pair of sequences in TABLE 11 or TABLE 12.

A conjugate or antibody construct can comprise a second binding domain that specifically binds CD40. A conjugate can comprise a second binding domain that is a CD40 agonist. A conjugate or antibody construct can comprise a second binding domain that binds CD40, wherein the second binding domain comprises at least 80% sequence identity to: a) HCDR1 comprising an amino acid sequence of SEQ ID NO: 3, HCDR2 comprising an amino acid sequence of SEQ ID NO: 4, HCDR3 comprising an amino acid sequence of SEQ ID NO: 5, LCDR1 comprising an amino acid sequence of SEQ ID NO: 8, LCDR2 comprising an amino acid sequence of SEQ ID NO: 9, and LCDR3 comprising an amino acid sequence of SEQ ID NO: 10; b) HCDR1 comprising an amino acid sequence of SEQ ID NO: 582, HCDR2 comprising an amino acid sequence of SEQ ID NO: 583, HCDR3 comprising an amino acid sequence of SEQ ID NO: 584, LCDR1 comprising an amino acid sequence of SEQ ID NO: 587, LCDR2 comprising an amino acid sequence of SEQ ID NO: 588, and LCDR3 comprising an amino acid sequence of SEQ ID NO: 589; c) HCDR1 comprising an amino acid sequence of SEQ ID NO: 592, HCDR2 comprising an amino acid sequence of SEQ ID NO: 593, HCDR3 comprising an amino acid sequence of SEQ ID NO: 594, LCDR1 comprising an amino acid sequence of SEQ ID NO: 597, LCDR2 comprising an amino acid sequence of SEQ ID NO: 598, and LCDR3 comprising an amino acid sequence of SEQ ID NO: 599; d) HCDR1 comprising an amino acid sequence of SEQ ID NO: 602, HCDR2 comprising an amino acid sequence of SEQ ID NO: 603, HCDR3 comprising an amino acid sequence of SEQ ID NO: 604, LCDR1 comprising an amino acid sequence of SEQ ID NO: 607, LCDR2 comprising an amino acid sequence of SEQ ID NO: 608, and LCDR3 comprising an amino acid sequence of SEQ ID NO: 609; e) HCDR1 comprising an amino acid sequence of SEQ ID NO: 612, HCDR2 comprising an amino acid sequence of SEQ ID NO: 613, HCDR3 comprising an amino acid sequence of SEQ ID NO: 614, LCDR1 comprising an amino acid sequence of SEQ ID NO: 617, LCDR2 comprising an amino acid sequence of SEQ ID NO: 618, and LCDR3 comprising an amino acid sequence of SEQ ID NO: 619; f) HCDR1 comprising an amino acid sequence of SEQ ID NO: 622, HCDR2 comprising an amino acid sequence of SEQ ID NO: 623, HCDR3 comprising an amino acid sequence of SEQ ID NO: 624, LCDR1 comprising an amino acid sequence of SEQ ID NO: 627, LCDR2 comprising an amino acid sequence of SEQ ID NO: 628, and LCDR3 comprising an amino acid sequence of SEQ ID NO: 629; or g) HCDR1 comprising an amino acid sequence of SEQ ID NO: 632, HCDR2 comprising an amino acid sequence of SEQ ID NO: 633, HCDR3 comprising an amino acid sequence of SEQ ID NO: 634, LCDR1 comprising an amino acid sequence of SEQ ID NO: 637, LCDR2 comprising an amino acid sequence of SEQ ID NO: 638, and LCDR3 comprising an amino acid sequence of SEQ ID NO: 639.

A conjugate or antibody construct can comprise a second binding domain that specifically binds DC-SIGN. A conjugate or antibody construct can comprise a second binding domain that binds DC-SIGN, wherein the second binding domain comprises at least 80% sequence identity to: a) HCDR1 comprising an amino acid sequence of SEQ ID NO: 640, HCDR2 comprising an amino acid sequence of SEQ ID NO: 641, HCDR3 comprising an amino acid sequence of SEQ ID NO: 642, LCDR1 comprising an amino acid sequence of SEQ ID NO: 643, LCDR2 comprising an amino acid sequence of SEQ ID NO: 644, and LCDR3 comprising an amino acid sequence of SEQ ID NO: 645; b) HCDR1 comprising an amino acid sequence of SEQ ID NO: 646, HCDR2 comprising an amino acid sequence of SEQ ID NO: 647, HCDR3 comprising an amino acid sequence of SEQ ID NO: 648, LCDR1 comprising an amino acid sequence of SEQ ID NO: 649, LCDR2 comprising an amino acid sequence of SEQ ID NO: 650, and LCDR3 comprising an amino acid sequence of SEQ ID NO: 651; or c) HCDR1 comprising an amino acid sequence of SEQ ID NO: 652, HCDR2 comprising an amino acid sequence of SEQ ID NO: 653, HCDR3 comprising an amino acid sequence of SEQ ID NO: 654, LCDR1 comprising an amino acid sequence of SEQ ID NO: 655, LCDR2 comprising an amino acid sequence of SEQ ID NO: 656, and LCDR3 comprising an amino acid sequence of SEQ ID NO: 657.

A conjugate or antibody construct can comprise a second binding domain that specifically binds DEC-205. A conjugate or antibody construct comprising a second binding domain that binds DEC-205 can comprise at least 80% sequence identity to: a) HCDR1 comprising an amino acid sequence of SEQ ID NO: 234, HCDR2 comprising an amino acid sequence of SEQ ID NO: 235, HCDR3 comprising an amino acid sequence of SEQ ID NO: 236, LCDR1 comprising an amino acid sequence of SEQ ID NO: 239, LCDR2 comprising an amino acid sequence of SEQ ID NO: 240, and LCDR3 comprising an amino acid sequence of SEQ ID NO: 241; or b) HCDR1 comprising an amino acid sequence of SEQ ID NO: 247, HCDR2 comprising an amino acid sequence of SEQ ID NO: 248, HCDR3 comprising an amino acid sequence of SEQ ID NO: 249, LCDR1 comprising an amino acid sequence of SEQ ID NO: 252, LCDR2 comprising an amino acid sequence of SEQ ID NO: 253, and LCDR3 comprising an amino acid sequence of SEQ ID NO: 254.

A conjugate or antibody construct can comprise a second binding domain comprising one or more variable domains. A conjugate or antibody construct can comprise a second binding domain comprising a light chain variable domain (VL domain). A second binding domain can comprise at least 80% sequence identity to any VL sequence in TABLE 13. A conjugate or antibody construct can comprise a second binding domain comprising a heavy chain variable domain. A second binding domain can comprise at least 80% sequence identity to any VH sequence in TABLE 13. A second binding domain can comprise at least 80% sequence identity to any sequence in TABLE 13. A second binding domain can comprise at least 80% sequence identity to any VL sequence in TABLE 13 or TABLE 14. A conjugate or antibody construct can comprise a second binding domain comprising a heavy chain variable domain. A second binding domain can comprise at least 80% sequence identity to any VH sequence in TABLE 13 or TABLE 14. A second binding domain can comprise at least 80% sequence identity to any sequence in TABLE 13 or TABLE 14.

A conjugate or antibody construct can comprise a second binding domain that specifically binds CD40. A conjugate or antibody construct can comprise a second binding domain that is a CD40 agonist. A conjugate or antibody construct can comprise a second binding domain that binds CD40, wherein the second binding domain comprises: a) a VH sequence having at least 80% sequence identity to an amino acid sequence of SEQ ID NO: 2, and a VL sequence having at least 80% sequence identity to an amino acid sequence of SEQ ID NO: 7; b) a VH sequence having at least 80% sequence identity to an amino acid sequence of SEQ ID NO: 581, and a VL sequence having at least 80% sequence identity to an amino acid sequence of SEQ ID NO: 586; c) a VH sequence having at least 80% sequence identity to an amino acid sequence of SEQ ID NO: 591, and a VL sequence having at least 80% sequence identity to an amino acid sequence of SEQ ID NO: 596; d) a VH sequence having at least 80% sequence identity to an amino acid sequence of SEQ ID NO: 601, and a VL sequence having at least 80% sequence identity to an amino acid sequence of SEQ ID NO: 606; e) a VH sequence having at least 80% sequence identity to an amino acid sequence of SEQ ID NO: 611, and a VL sequence having at least 80% sequence identity to an amino acid sequence of SEQ ID NO: 616; f) a VH sequence having at least 80% sequence identity to an amino acid sequence of SEQ ID NO: 621, and a VL sequence having at least 80% sequence identity to an amino acid sequence of SEQ ID NO: 626; g) a VH sequence having at least 80% sequence identity to an amino acid sequence of SEQ ID NO: 631, and a VL sequence having at least 80% sequence identity to an amino acid sequence of SEQ ID NO: 636.

A conjugate or antibody construct can comprise a second binding domain that specifically binds DEC-205. A conjugate or antibody construct can comprise a second binding domain that binds DEC-205, wherein the second binding domain comprises: a) a VH sequence having at least 80% sequence identity to an amino acid sequence of SEQ ID NO: 233, and a VL sequence having at least 80% sequence identity to an amino acid sequence of SEQ ID NO: 238; or b) a VH sequence having at least 80% sequence identity to an amino acid sequence of SEQ ID NO: 246, and a VL sequence having at least 80% sequence identity to an amino acid sequence of SEQ ID NO: 251.

A conjugate or antibody construct can comprise a second binding domain that specifically binds CD36 mannose scavenger receptor 1. A conjugate or antibody construct can comprise a second binding domain that binds CD36 mannose scavenger receptor 1, wherein the second binding domain comprises a VH sequence having at least 80% sequence identity to an amino acid sequence of SEQ ID NO: 658, and a VL sequence having at least 80% sequence identity to an amino acid sequence of SEQ ID NO: 659.

A conjugate or antibody construct can comprise a second binding domain that specifically binds CLEC9A. A conjugate or antibody construct can comprise a second binding domain that binds CLEC9A, wherein the second binding domain comprises a VH sequence having at least 80% sequence identity to an amino acid sequence of SEQ ID NO: 660, and a VL sequence having at least 80% sequence identity to an amino acid sequence of SEQ ID NO: 661.

A conjugate or antibody construct can comprise a second binding domain and an Fc domain, wherein the second binding domain and the Fc domain comprise an antibody. A conjugate or antibody construct can comprise a heavy chain and a light chain that target a molecule expressed by an immune cell such as an antigen presenting cell. A conjugate or antibody construct can comprise an antibody light chain. A conjugate or antibody construct can comprise a light chain comprising at least 80% sequence identity to any light chain sequence in TABLE 15. A conjugate or antibody construct can comprise an antibody heavy chain. A conjugate or antibody construct can comprise a heavy chain comprising at least 80% sequence identity to any heavy chain sequence in TABLE 15. A conjugate or antibody construct can comprise at least 80% sequence identity to any sequence in TABLE 15. A conjugate or antibody construct can comprise a light chain comprising at least 80% sequence identity to any light chain sequence in TABLE 15 or TABLE 16. A conjugate or antibody construct can comprise an antibody heavy chain. A conjugate or antibody construct can comprise a heavy chain comprising at least 80% sequence identity to any heavy chain sequence in TABLE 15 or TABLE 16. A conjugate or antibody construct can comprise at least 80% sequence identity to any sequence in TABLE 15 or TABLE 16.

A conjugate or antibody construct can comprise a heavy chain and a light chain that target a molecule expressed by an immune cell such as an antigen presenting cell. A conjugate or antibody construct can comprise a first binding domain and an Fc domain, wherein the first binding domain and the Fc domain comprise an antibody. A conjugate or antibody construct can comprise an anti-CD40 antibody, the conjugate comprising: a) a heavy chain sequence having at least 80% sequence identity to an amino acid sequence of SEQ ID NO: 1 and a light chain sequence having at least 80% sequence identity to an amino acid sequence of SEQ ID NO: 6; b) a heavy chain sequence having at least 80% sequence identity to an amino acid sequence of SEQ ID NO: 577 or SEQ ID NO: 578, and a light chain sequence having at least 80% sequence identity to an amino acid sequence of SEQ ID NO: 579; c) a heavy chain sequence having at least 80% sequence identity to an amino acid sequence of SEQ ID NO: 580, and a light chain sequence having at least 80% sequence identity to an amino acid sequence of SEQ ID NO: 585; d) a heavy chain sequence having at least 80% sequence identity to an amino acid sequence of SEQ ID NO: 590, and a light chain sequence having at least 80% sequence identity to an amino acid sequence of SEQ ID NO: 595; e) a heavy chain sequence having at least 80% sequence identity to an amino acid sequence of SEQ ID NO: 600, and a light chain sequence having at least 80% sequence identity to an amino acid sequence of SEQ ID NO: 605; f) a heavy chain sequence having at least 80% sequence identity to an amino acid sequence of SEQ ID NO: 610, and a light chain sequence having at least 80% sequence identity to an amino acid sequence of SEQ ID NO: 615; g) a heavy chain sequence having at least 80% sequence identity to an amino acid sequence of SEQ ID NO: 620, and a light chain sequence having at least 80% sequence identity to an amino acid sequence of SEQ ID NO: 625; or h) a heavy chain sequence having at least 80% sequence identity to an amino acid sequence of SEQ ID NO: 630, and a light chain sequence having at least 80% sequence identity to an amino acid sequence of SEQ ID NO: 635.

A conjugate or antibody construct can comprise a first binding domain and an Fc domain, wherein the first binding domain and the Fc domain comprise an antibody. A conjugate or antibody construct can comprise an anti-DEC-205 antibody, the conjugate or antibody construct comprising: a) a heavy chain sequence having at least 80% sequence identity to an amino acid sequence of SEQ ID NO: 232, and a light chain sequence having at least 80% sequence identity to an amino acid sequence of SEQ ID NO: 237; or b) a heavy chain sequence having at least 80% sequence identity to an amino acid sequence of SEQ ID NO: 245, and a light chain sequence having at least 80% sequence identity to an amino acid sequence of SEQ ID NO: 250.

A conjugate or antibody construct can comprise a first binding domain and an Fc domain, wherein the first binding domain and the Fc domain comprise an antibody. A conjugate or antibody construct can comprise an anti-CLEC12A antibody, the conjugate comprising: a) a heavy chain sequence having at least 80% sequence identity to an amino acid sequence of SEQ ID NO: 662, and a light chain sequence having at least 80% sequence identity to an amino acid sequence of SEQ ID NO: 665; b) a heavy chain sequence having at least 80% sequence identity to an amino acid sequence of SEQ ID NO: 663, and a light chain sequence having at least 80% sequence identity to an amino acid sequence of SEQ ID NO: 665; or c) a heavy chain sequence having at least 80% sequence identity to an amino acid sequence of SEQ ID NO: 664, and a light chain sequence having at least 80% sequence identity to an amino acid sequence of SEQ ID NO: 665.

A conjugate or antibody construct can comprise a first binding domain and an Fc domain, wherein the first binding domain and the Fc domain comprise an antibody. A conjugate or antibody construct can comprise an anti-BDCA-2 antibody, the conjugate comprising: a) a heavy chain sequence having at least 80% sequence identity to an amino acid sequence of SEQ ID NO: 666, and a light chain sequence having at least 80% sequence identity to an amino acid sequence of SEQ ID NO: 669; b) a heavy chain sequence having at least 80% sequence identity to an amino acid sequence of SEQ ID NO: 667, and a light chain sequence having at least 80% sequence identity to an amino acid sequence of SEQ ID NO: 670; or c) a heavy chain sequence having at least 80% sequence identity to an amino acid sequence of SEQ ID NO: 668, and a light chain sequence having at least 80% sequence identity to an amino acid sequence of SEQ ID NO: 671.

### Antibody-ScFv Fusion Protein Products

A conjugate or antibody construct can comprise a first binding domain, a second binding domain, and an Fc domain, wherein the first binding domain and the second binding domain are attached to the Fc domain. The first binding domain and the second binding domain can be attached to the Fc domain as a fusion peptide (also referred to as a fusion protein). The first binding domain can be attached to the Fc domain at an N-terminal end of the Fc domain, wherein the second binding domain can be attached to the Fc domain at a C-terminal end. The first binding domain can be attached to the Fc domain at an N-terminal end of the Fc domain, wherein the second binding domain can be attached to the Fc domain at a C-terminal end via a polypeptide linker ranging from 10 to 25 amino acids comprising the sequence [G4S]n where n=2 to 5 (SEQ ID NO: 1330). Alternatively, the first binding domain can be attached to the Fc domain at a C-terminal end of the Fc domain, wherein the second binding domain can be attached to the Fc domain at an N-terminal end. A second binding domain and an Fc domain can comprise an antibody and a first binding domain can comprise a single chain variable fragment (scFv). A single chain variable fragment can comprise a heavy chain variable domain and a light chain variable domain of an antibody. The first binding domain of the fusion peptide can be attached to the second binding domain at a heavy chain variable domain of the single chain variable fragment of the first binding domain (HL orientation). Alternatively, the first binding domain of the fusion peptide can be attached to the second binding domain at a light chain variable domain of the single chain variable fragment of the first binding domain (LH orientation). In either orientation, the first binding domain and the second binding domain can be attached via a polypeptide linker varying in length from 15 to 25 amino acids, wherein the linker comprises the sequence [G4S]n where n=3 to 5 (SEQ ID NO: 1331).

Alternatively, a first binding domain and an Fc domain can comprise an antibody and the second binding domain can comprise a single chain variable fragment (scFv). The second binding domain of the fusion peptide can be attached to the first binding domain at a heavy chain variable domain of the single chain variable fragment of the first binding domain (HL orientation). Alternatively, the second binding domain of the fusion peptide can be attached to the first binding domain at a light chain variable domain of the single chain variable fragment of the first binding domain (LH orientation).

A conjugate or antibody construct can comprise a first binding domain and a second binding domain, wherein the second binding domain can be attached to the first binding domain. The conjugate or antibody construct can comprise an antibody comprising a light chain and a heavy chain. The first binding domain can comprise a Fab fragment of the light and heavy chains. The second binding domain can be attached to the light chain at a C-terminus or C-terminal end of the light chain as a fusion peptide. The second binding domain can comprise a single chain variable fragment (scFv).

A conjugate or antibody construct can comprise a first binding domain, a second binding domain, and an Fc domain, wherein the first binding domain and the second binding domain are attached to the Fc domain as a fusion peptide. The second binding domain of the fusion peptide can specifically bind to an antigen with at least 80% homology to CD40. The second binding domain of the fusion peptide can be a CD40 agonist. The first binding domain of the fusion peptide can target a tumor antigen. The conjugate or antibody construct can comprise a fusion peptide comprising a heavy chain (HC) attached to a single chain variable fragment. The conjugate or antibody construct comprising the fusion peptide can comprise a sequence in TABLE 9. The conjugate or antibody construct comprising the fusion peptide can comprise at least 80% sequence identity to any sequence in TABLE 9. The conjugate or antibody construct comprising the fusion peptide can comprise at least 80% sequence identity to a sequence of a heavy chain CD40 monoclonal antibody (mAb) with tumor ScFv in TABLE 9. The conjugate or antibody construct can comprise a fusion peptide comprising a sequence of a heavy chain CD40 mAb with tumor ScFv in TABLE 9 and a light chain comprising SEQ ID NO: 6. The conjugate or antibody construct can comprise a fusion peptide comprising at least 80% sequence identity to a sequence of a heavy chain CD40 mAb with tumor ScFv in TABLE 9 and a light chain comprising at least 80% sequence identity to SEQ ID NO: 6. The conjugate or antibody construct comprising the fusion peptide can comprise a sequence of a heavy chain tumor mAb with CD40 ScFv in TABLE 9. The conjugate or construct comprising the fusion peptide can comprise at least 80% sequence identity to any sequence of a heavy chain tumor mAb with CD40 ScFv in TABLE 9. The conjugate or antibody construct comprising the fusion peptide can comprise a sequence of a heavy chain tumor antigen mAb with CD40 ScFv in TABLE 9, and a light chain mAb for the tumor antigen in TABLE 7. The conjugate or antibody construct comprising the fusion peptide can comprise at least 80% sequence identity to any sequence of a heavy chain tumor antigen mAb with CD40 ScFv in TABLE 9, and at least 80% sequence identity to a light chain mAb for the tumor antigen in TABLE 7.

The conjugate or antibody construct comprising the fusion peptide can comprise a sequence in TABLE 9 or TABLE 10. The conjugate or antibody construct comprising the fusion peptide can comprise at least 80% sequence identity to any sequence in TABLE 9 or TABLE 10. The conjugate or antibody construct comprising the fusion peptide can comprise at least 80% sequence identity to a sequence of a heavy chain CD40 monoclonal antibody (mAb) with tumor ScFv in TABLE 9 or TABLE 10. The conjugate or antibody construct can comprise a fusion peptide comprising a sequence of a heavy chain CD40 mAb with tumor ScFv in TABLE 9 or TABLE 10 and a light chain comprising SEQ ID NO: 6. The conjugate or antibody construct can comprise a fusion peptide comprising at least 80% sequence identity to a sequence of a heavy chain CD40 mAb with tumor ScFv in TABLE 9 or TABLE 10 and a light chain comprising at least 80% sequence identity to SEQ ID NO: 6. The conjugate or antibody construct comprising the fusion peptide can comprise a sequence of a heavy chain tumor mAb with CD40 ScFv in TABLE 9 or TABLE 10. The conjugate or construct comprising the fusion peptide can comprise at least 80% sequence identity to any sequence of a heavy chain tumor mAb with CD40 ScFv in TABLE 9 or TABLE 10. The conjugate or antibody construct comprising the fusion peptide can comprise a sequence of a heavy chain tumor antigen mAb with CD40 ScFv in TABLE 9 or TABLE 10, and a light chain mAb for the tumor antigen in TABLE 7 or TABLE 8. The conjugate or antibody construct comprising the fusion peptide can comprise at least 80% sequence identity to any sequence of a heavy chain tumor antigen mAb with CD40 ScFv in TABLE 9 or TABLE 10, and at least 80% sequence identity to a light chain mAb for the tumor antigen in TABLE 7 or TABLE 8.

A conjugate or antibody construct can comprise a first binding domain and a second binding domain, wherein the second binding domain can be attached to the first binding domain. A conjugate or antibody construct can comprise a first binding domain, a second binding domain, and an Fc domain, wherein the second binding domain can be attached to the first binding domain. The second binding domain can be attached at a C-terminal end of the first binding domain as a fusion peptide. The first binding domain can comprise a Fab fragment comprising a light chain, wherein the second binding domain can be attached at a C-terminal end of the light chain as a fusion peptide. The second binding domain of the fusion peptide can comprise a single chain variable fragment (scFv). The second binding domain of the fusion peptide can be attached to the first binding domain at a heavy chain variable domain of the single chain variable fragment of the first binding domain (HL orientation). Alternatively, the second binding domain of the fusion peptide can be attached to the first binding domain at a light chain variable domain of the single chain variable fragment of the first binding domain (LH orientation). For example, a fusion peptide comprising a light chain of an anti-CEA antibody attached to an anti-CD40 scFv in the LH orientation can be illustrated by SEQ ID NO: 842. All fusion sequences comprising an scFv sequence are in the HL orientation unless indicated otherwise (e.g., sequence name recites “(LH)” indicating light heavy orientation).

The first binding domain of the fusion peptide can target a tumor antigen. The second binding domain of the fusion peptide can target an APC antigen. The second binding domain of the fusion peptide can target CD40. The first binding domain can comprise a Fab fragment comprising a light chain, wherein the second binding domain is attached at a C-terminal end of the light chain as a fusion peptide. The conjugate or antibody construct comprising the fusion peptide can comprise a sequence in TABLE 18. The conjugate or antibody construct comprising the fusion peptide can comprise at least 80% sequence identity to any sequence in TABLE 18. The conjugate or antibody construct comprising the fusion peptide can comprise a sequence of a light chain CD40 mAb with tumor ScFv in TABLE 18. The conjugate or antibody construct comprising the fusion peptide can comprise at least 80% sequence identity to any sequence of a light chain CD40 mAb with tumor ScFv in TABLE 18. The conjugate or antibody construct can comprise a fusion peptide comprising a sequence of a light chain CD40 mAb with tumor ScFv in TABLE 11 and a heavy chain comprising SEQ ID NO: 1. The conjugate or antibody construct can comprise a fusion peptide comprising at least 80% sequence identity to any sequence of a light chain CD40 mAb with tumor ScFv in TABLE 11 and a heavy chain comprising at least 80% sequence identity to SEQ ID NO: 1. The conjugate or antibody construct comprising the fusion peptide can comprise a sequence of a light chain tumor mAb with CD40 ScFv in TABLE 18. The conjugate or antibody construct comprising the fusion peptide can comprise at least 80% sequence identity to any sequence of a light chain tumor mAb with CD40 ScFv in TABLE 18. The conjugate or antibody construct can comprise a fusion peptide comprising a sequence of a light chain tumor antigen mAb with CD40 ScFv in TABLE 18, and a heavy chain mAb for the tumor antigen in TABLE 7. The conjugate or antibody construct can comprise a fusion peptide comprising at least 80% sequence identity to any sequence of a light chain tumor antigen mAb with CD40 ScFv in TABLE 18, and at least 80% sequence identity to a heavy chain mAb for the tumor antigen in TABLE 7. The conjugate or antibody construct can comprise a fusion peptide comprising a sequence of a light chain tumor antigen mAb with CD40 ScFv in TABLE 18, and a heavy chain mAb for the tumor antigen in TABLE 7 or TABLE 8. The conjugate or antibody construct can comprise a fusion peptide comprising at least 80% sequence identity to any sequence of a light chain tumor antigen mAb with CD40 ScFv in TABLE 18, and at least 80% sequence identity to a heavy chain mAb for the tumor antigen in TABLE 7 or TABLE 8.

A conjugate or antibody construct can comprise a first binding domain, a second binding domain, and an Fc domain, wherein the first binding domain and the second binding domain are attached to the Fc domain as a fusion peptide. The first binding domain of the fusion peptide can specifically bind to an antigen with at least 80% homology to DEC-205. The second binding domain of the fusion peptide can target a tumor antigen. The conjugate or antibody construct can comprise a fusion peptide comprising a heavy chain attached to a single chain variable fragment. The conjugate or antibody construct comprising the fusion peptide can comprise a sequence in TABLE 17. The conjugate or antibody construct comprising the fusion peptide can comprise at least 80% sequence identity to any sequence in TABLE 17. The conjugate or antibody construct comprising the fusion peptide can comprise a sequence of a heavy chain DEC-205 mAb with tumor ScFv in TABLE 17. The conjugate or antibody construct comprising the fusion peptide can comprise at least 80% sequence identity to any sequence of a heavy chain DEC-205 mAb with tumor ScFv in TABLE 17. The conjugate or antibody construct can comprise a fusion peptide comprising a sequence of a heavy chain DEC-205 mAb with tumor ScFv in TABLE 10 and a peptide comprising SEQ ID NO: 237. The conjugate or antibody construct can comprise a fusion peptide comprising at least 80% sequence identity to any sequence of a heavy chain DEC-205 mAb with tumor ScFv in TABLE 17 and a peptide comprising at least 80% sequence identity to SEQ ID NO: 237. The conjugate or antibody construct comprising the fusion peptide can comprise a sequence of a heavy chain tumor antigen mAb with CD40 ScFv in TABLE 17. The conjugate or antibody construct comprising the fusion peptide can comprise at least 80% sequence identity to any sequence of a heavy chain tumor antigen mAb with CD40 ScFv in TABLE 17. The conjugate or antibody construct comprising the fusion peptide can comprise a sequence of a heavy chain tumor antigen mAb with CD40 ScFv in TABLE 17, and a heavy chain mAb for the tumor antigen in TABLE 7. The conjugate or antibody construct comprising the fusion peptide can comprise at least 80% sequence identity to any sequence of a heavy chain tumor antigen mAb with CD40 ScFv in TABLE 17, and at least 80% sequence identity to a heavy chain mAb for the tumor antigen in TABLE 7. The conjugate or antibody construct comprising the fusion peptide can comprise a sequence of a heavy chain tumor antigen mAb with CD40 ScFv in TABLE 17, and a heavy chain mAb for the tumor antigen in TABLE 7 or TABLE 8. The conjugate or antibody construct comprising the fusion peptide can comprise at least 80% sequence identity to any sequence of a heavy chain tumor antigen mAb with CD40 ScFv in TABLE 17, and at least 80% sequence identity to a heavy chain mAb for the tumor antigen in TABLE 7 or TABLE 8.

The second binding domain of the fusion peptide can target DEC-205. The conjugate or antibody construct comprising the fusion peptide can comprise a sequence in TABLE 19. The conjugate or antibody construct comprising the fusion peptide can comprise at least 80% sequence identity to any sequence in TABLE 19. The conjugate or antibody construct comprising the fusion peptide can comprise a sequence of a light chain DEC-205 mAb with tumor ScFv in TABLE 19. The conjugate or antibody construct comprising the fusion peptide can comprise at least 80% sequence identity to any sequence of a light chain DEC-205 mAb with tumor ScFv in TABLE 19. The conjugate or antibody construct comprising a fusion peptide can comprise a sequence of a light chain DEC-205 mAb with tumor ScFv in TABLE 19 and SEQ ID NO: 237. The conjugate or antibody construct comprising a fusion peptide can comprise at least 80% sequence identity to any sequence of a light chain DEC-205 mAb with tumor ScFv in TABLE 19 and at least 80% sequence identity to SEQ ID NO: 237. The conjugate or antibody construct comprising the fusion peptide can comprise a sequence of a light chain tumor mAb with DEC-205 ScFv in TABLE 19. The conjugate or antibody construct comprising the fusion peptide can comprise at least 80% sequence identity to any sequence of a light chain tumor mAb with DEC-205 ScFv in TABLE 19. The conjugate or antibody construct comprising a fusion peptide can comprise a sequence of a light chain tumor antigen mAb with DEC-205 ScFv in TABLE 19, and a heavy chain mAb for the tumor antigen in TABLE 7. The conjugate or antibody construct comprising a fusion peptide can comprise at least 80% sequence identity to any sequence of a light chain tumor antigen mAb with DEC-205 ScFv in TABLE 19, and at least 80% sequence identity to a heavy chain mAb for the tumor antigen in TABLE 7. The conjugate or antibody construct comprising a fusion peptide can comprise a sequence of a light chain tumor antigen mAb with DEC-205 ScFv in TABLE 19, and a heavy chain mAb for the tumor antigen in TABLE 7 or TABLE 8. The conjugate or antibody construct comprising a fusion peptide can comprise at least 80% sequence identity to any sequence of a light chain tumor antigen mAb with DEC-205 ScFv in TABLE 19, and at least 80% sequence identity to a heavy chain mAb for the tumor antigen in TABLE 7 or TABLE 8.

The second binding domain of the fusion peptide can specifically bind to an antigen of an immune cell, such as an antigen presenting cell, (APC). The second binding domain of the fusion peptide can specifically bind to an antigen with at least 80% homology to CD40. The second binding domain of the fusion peptide can be a CD40 agonist. The second binding domain of the fusion peptide can specifically bind to an antigen with at least 80% homology to DEC-205. The second binding domain of the fusion peptide can specifically bind to an antigen with at least 80% homology to DC-SIGN. The second binding domain of the fusion peptide can specifically bind to an antigen with at least 80% homology to CD36 mannose scavenger receptor. The second binding domain of the fusion peptide can specifically bind to an antigen with at least 80% homology to CLEC12A. The second binding domain of the fusion peptide can specifically bind to an antigen with at least 80% homology to BDCA-2. The second binding domain of the fusion peptide can specifically bind to an antigen having an amino acid sequence with at least 80% or 100% homology to an amino acid sequence of CD40, CD47, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B The second binding domain of the fusion peptide can specifically bind to an antigen having an amino acid sequence with at least 80% or 100% homology to an amino acid sequence of CD40, CD47, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, TNFR2, or TREM2. The second binding domain of the fusion peptide can specifically bind to an antigen having an amino acid sequence with at least 80% or 100% homology to an amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B. The second binding domain of the fusion peptide can specifically bind to an antigen having an amino acid sequence with at least 80% or 100% homology to an amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, TNFR2, or TREM2. The first binding domain of the fusion peptide can target a tumor antigen. The first binding domain of the fusion peptide can target an antigen having an amino acid sequence with at least 80% or 100% homology to an amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. The first binding domain of the fusion peptide also can target an antigen having an amino acid sequence with at least 80% or 100% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6. In some embodiments, the first targeting domain can target an antigen having an amino acid sequence with at least 80% or 100% homology to the amino acid sequence of TROP2, CEA, MUC16, LRRC15, CLDN6, CLDN16, UPK1B, VTCN1 (B7-H4) or STRA6.

Alternatively, the second binding domain of the fusion peptide can target a tumor antigen. The second binding domain of the fusion peptide can target an antigen having an amino acid sequence with at least 80% or 100% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. The second binding domain of the fusion peptide also can target an antigen having an amino acid sequence with at least 80% or 100% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6. The first binding domain of the fusion peptide can specifically bind to an antigen having an amino acid sequence with at least 80% homology to the amino acid sequence of CD40. The first binding domain of the fusion peptide can be a CD40 agonist. The first binding domain of the fusion peptide can specifically bind to an antigen having an amino acid sequence with at least 80% homology to the amino acid sequence of DEC-205. The first binding domain of the fusion peptide can specifically bind to an antigen having an amino acid sequence with at least 80% or 100% homology to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B. The first binding domain of the fusion peptide can specifically bind to an antigen having an amino acid sequence with at least 80% homology to the amino acid sequence of DEC-205. The first binding domain of the fusion peptide can specifically bind to an antigen having an amino acid sequence with at least 80% or 100% homology to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, TNFR2, or TREM2.

In some embodiments, the first binding domain can specifically bind to an antigen having an amino acid sequence with at least 80% or 100% homology to the amino acid sequence of TROP2, CEA, MUC16, LRRC15, CLDN6, CLDN16, UPK1B, VTCN1 (B7-H4) and STRA6 and a second binding domain can target an antigen having an amino acid sequence with at least 80% or 100% homology to the amino acid sequence of CD40 or PD-L1.

A conjugate or antibody construct can comprise a first binding domain, a second binding domain, and a third binding domain. A conjugate or antibody construct can comprise a first binding domain, a second binding domain, a third binding domain, and an Fc domain. The first binding domain and the second binding domain can be attached to the Fc domain. The first and second binding domains are described herein throughout the specification. The first binding domain can be attached to the Fc domain at an N-terminal end of the Fc domain. The second binding domain can be attached at a C-terminal end of the Fc domain. The second binding domain can be attached at a C-terminal end of the Fc domain via a polypeptide linker having a length of 10 to 25 amino acid comprising the sequence [G4S]n where n=2 to 5 (SEQ ID NO: 1330). The third binding domain can be attached to a C-terminal end of the first binding domain. The third binding domain can be attached at a C-terminal end of the Fc domain via a polypeptide linker having a length of 10 to 25 amino acid comprising the sequence [G4S]n where n=2 to 5 (SEQ ID NO: 1330). The third binding domain can be attached to a C-terminal end of a light chain of the first binding domain. One or more of the first binding domain, the second binding domain, the third binding domain, and the Fc domain can be attached as a fusion peptide. The first binding domain can comprise a Fab fragment comprising a light chain, wherein the second binding domain is attached at a C-terminal end of the light chain as a fusion peptide. The second binding domain of the fusion peptide can comprise a single chain variable fragment (scFv). The second binding domain of the fusion peptide can be attached to the Fc domain at a heavy chain variable domain of the single chain variable fragment of the second binding domain (HL orientation). The second binding domain of the fusion peptide can be attached to the Fc domain at a light chain variable domain of the single chain variable fragment of the second binding domain (LH orientation). The third binding domain of the fusion peptide can comprise a single chain variable fragment (scFv). The conjugate or antibody construct can comprise a fusion peptide comprising the third binding domain attached to the first binding domain having at least 80% or 100% sequence identity to any sequence in TABLE 18 or TABLE 19. The third binding domain of the fusion peptide can be attached to the first binding domain at a heavy chain variable domain of the single chain variable fragment of the first binding domain (HL orientation). Alternatively, the third binding domain of the fusion peptide can be attached to the first binding domain at a light chain variable domain of the single chain variable fragment of the first binding domain (LH orientation). The third binding domain of the fusion peptide can target an antigen of an immune cell, such as an antigen presenting cell, (APC). The third binding domain of the fusion peptide can specifically bind to an antigen with at least 80% homology to the amino acid sequence of CD40. The third binding domain of the fusion peptide can be a CD40 agonist. The third binding domain of the fusion peptide can specifically bind to an antigen with at least 80% homology to the amino acid sequence of DEC-205. The third binding domain of the fusion peptide can specifically bind to an antigen having an amino acid sequence with at least 80% homology to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B. The third binding domain of the fusion peptide can specifically bind to an antigen having an amino acid sequence with at least 80% homology to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, TNFR2, or TREM2.

Alternatively, the third binding domain can target a tumor antigen. The third binding domain of the fusion peptide can target an antigen having an amino acid sequence with at least 80% or 100% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. The third binding domain of the fusion peptide can target an antigen having an amino acid sequence with at least 80% or 100% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6.

A conjugate or antibody construct can comprise a first binding domain targeting CD40 and a second binding domain targeting DEC-205. Alternatively, a conjugate or antibody construct can comprise a first binding domain targeting DEC-205 and a second binding domain targeting CD40. A conjugate or antibody construct can comprise a first binding domain, a second binding domain, and an Fc domain. The first binding domain and the second binding domain can be attached to the Fc domain. The first binding domain can be attached to the Fc domain at an N-terminal end of the Fc domain, wherein the second binding domain is attached to the Fc domain at a C-terminal end of the Fc domain. Alternatively, second binding domain can be attached to the Fc domain at an N-terminal end of the Fc domain, wherein the first binding domain is attached to the Fc domain at a C-terminal end of the Fc domain. A conjugate or antibody construct can comprise a fusion peptide comprising a first binding domain targeting CD40 and a second binding domain targeting DEC-205. The fusion peptide can comprise at least 80% or 100% sequence identity to any sequence in TABLE 20.

Additionally, conjugates or antibody constructs containing the sequences referenced in TABLES 3-20 can have a dissociation constant (Kd) that is less than 10 nM for the antigen of the first binding domain. The conjugates or antibody constructs containing the sequences referenced in TABLES 3-20 can have a dissociation constant (Kd) that is less than 10 nM for the antigen of the second binding domain. The conjugates or antibody constructs containing the sequences referenced in TABLES 3-20 can have a dissociation constant (Kd) that is less than 10 nM for the antigen of the third binding domain. The conjugates or antibody constructs can have a dissociation constant (Kd) for the antigen of the first binding domain that is less than 1 nM, less than 100 pM, less than 10 pM, less than 1 pM, or less than 0.1 pM. The conjugates or antibody constructs can have a dissociation constant (Kd) for the antigen of the second binding domain that is less than 1 nM, less than 100 pM, less than 10 pM, less than 1 pM, or less than 0.1 pM. The conjugates or antibody constructs can have a dissociation constant (Kd) for the antigen of the third binding domain that is less than 1 nM, less than 100 pM, less than 10 pM, less than 1 pM, or less than 0.1 pM.

An anti-CD40 light chain can be expressed with its corresponding anti-CD40 heavy chain or fragment thereof. The corresponding anti-CD40 heavy chain or fragment thereof can be a heavy chain or fragment that when paired with the anti-CD40 light chain, can bind to a CD40 antigen. The anti-CD40 light chain can also be expressed with its corresponding anti-CD40 heavy chain or fragment thereof to form an anti-CD40 antibody or fragment thereof. The anti-CD40 antibody or fragment thereof can be purified, and can be combined with a pharmaceutically acceptable carrier.

An anti-DEC-205 light chain can be expressed with its corresponding anti-DEC-205 heavy chain or fragment thereof. The corresponding anti-DEC-205 heavy chain or fragment thereof can be a heavy chain or fragment that when paired with the anti-DEC-205 light chain, can bind to a DEC-205 antigen. The anti-DEC-205 light chain can also be expressed with its corresponding anti-DEC-205 heavy chain or fragment thereof to form an anti-DEC-205 antibody or fragment thereof. The anti-DEC-205 antibody or fragment thereof can be purified, and can be combined with a pharmaceutically acceptable carrier.

An anti-tumor antigen light chain can be expressed with an anti-tumor antigen heavy chain or fragment thereof. The anti-tumor antigen light chain can also expressed with an anti-tumor antigen heavy chain or fragment thereof to form an anti-tumor antigen antibody or fragment thereof. The anti-tumor antibody or fragment thereof can be purified, and can be combined with a pharmaceutically acceptable carrier.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG isotype. A heavy chain of an anti-CD40 antibody can be dacetuzumab.

A conjugate or antibody construct can comprise an antibody light chain. A light chain can be a light chain of an anti-CD40 antibody which can bind to a CD40 antigen. A light chain of an anti-CD40 antibody can be dacetuzumab.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG4 isotype. A heavy chain of an anti-CD40 antibody can be bleselumab.

A conjugate or antibody construct can comprise an antibody light chain. A light chain can be a light chain of an anti-CD40 antibody which can bind to a CD40 antigen. A light chain of an anti-CD40 antibody can be bleselumab.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG isotype. A heavy chain of an anti-CD40 antibody can be lucatumumab.

A conjugate or antibody construct can comprise an antibody light chain. A light chain can be a light chain of an anti-CD40 antibody which can bind to a CD40 antigen. A light chain of an anti-CD40 antibody can be lucatumumab.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG isotype. A heavy chain of an anti-CD40 antibody can be ADC-1013.

A conjugate or antibody construct can comprise an antibody light chain. A light chain can be a light chain of an anti-CD40 antibody which can bind to a CD40 antigen. A light chain of an anti-CD40 antibody can be ADC-1013.

A conjugate or antibody construct can comprise an antibody heavy chain. A heavy chain can be a heavy chain of an anti-CD40 antibody which can bind to a CD40 antigen. A heavy chain of an anti-CD40 antibody can be the humanized rabbit antibody APX005.

A conjugate or antibody construct can comprise an antibody light chain. A light chain can be a light chain of an anti-CD40 antibody which can bind to a CD40 antigen. A light chain of an anti-CD40 antibody can be the humanized rabbit antibody APX005.

A conjugate or antibody construct can comprise an antibody heavy chain. A heavy chain can be a heavy chain of an anti-CD40 antibody which can bind to a CD40 antigen. A heavy chain of an anti-CD40 antibody can be Chi Lob 7/4.

A conjugate or antibody construct can comprise an antibody light chain. A light chain can be a light chain of an anti-CD40 antibody which can bind to a CD40 antigen. A light chain of an anti-CD40 antibody can be Chi Lob 7/4.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG isotype. A heavy chain of an anti-CD40 antibody can be SBT-040-G1WT.

A conjugate or antibody construct can comprise an antibody heavy chain. A heavy chain can be a heavy chain of an anti-CD40 antibody which can bind to a CD40 antigen. A heavy chain of an anti-CD40 antibody can be an IgG isotype. A heavy chain of an anti-CD40 antibody can be SBT-040 VH-hIgG1 wt.

A heavy chain of an anti-CD40 antibody can be an IgG2 isotype. A heavy chain of an anti-CD40 antibody can be SBT-040-G2.

A conjugate or antibody construct can comprise an antibody with modifications occurring at least at one amino acid residue. Modifications can be substitutions, additions, mutations, deletions, or the like. An antibody modification can be an insertion of an unnatural amino acid.

A conjugate or antibody construct can comprise a light chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A conjugate or antibody construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A heavy chain can be the heavy chain of an anti-CD40 antibody which can bind to the CD40 antigen.

A conjugate or antibody construct can comprise an Fc domain of an IgG isotype. A conjugate or antibody construct can comprise an Fc domain of an IgG2 isotype. A conjugate or antibody construct can comprise an Fc domain of an IgG3 isotype. A conjugate can comprise an Fc domain of an IgG4 isotype. A conjugate or antibody construct can have a hybrid isotype comprising constant regions from two or more isotypes. A conjugate or antibody construct can be an anti-CD40 antibody, in which the anti-CD40 antibody can be a monoclonal human antibody comprising a wild-type sequence of an IgG isoform, in particular, at an Fc region of the antibody.

Conjugates and antibody constructs disclosed herein can be non-natural, designed, and/or engineered. Conjugates and antibody constructs disclosed herein can be non-natural, designed, and/or engineered scaffolds comprising an antigen binding domain. Conjugates and antibody constructs disclosed herein can be non-natural, designed, and/or engineered antibodies. Conjugates and antibody constructs can include monoclonal antibodies. Conjugates and antibody constructs can comprise human antibodies. Conjugates and antibody constructs can comprise humanized antibodies. Conjugates and antibody constructs can comprise monoclonal humanized antibodies. Conjugates and antibody constructs can comprise recombinant antibodies.

The Kd for binding of the Fc domain to an Fc receptor of a conjugate or antibody construct as described herein can increase when the tumor antigen binding domain is bound to its tumor antigen as compared to the Kd for binding of the Fc domain to an Fc receptor of a conjugate or antibody construct as described herein when the tumor antigen binding domain is not bound to its tumor antigen. For example, a conjugate or antibody construct as described herein can have a Kd for binding of the Fc domain to an Fc receptor in the presence of the binding domain that binds to a molecule on an immune cell, such as an antigen presenting cell, and the tumor targeting binding domain when the tumor targeting binding domain is bound to its tumor antigen that can be greater than or greater than about 100 nM. The Kd for binding of the Fc domain to an Fc receptor in the presence of the binding domain that binds to a molecule on an immune cell, such as an antigen presenting cell, and the tumor targeting binding domain when the tumor targeting binding domain is bound to its tumor antigen can be or can be about 100 nM, 200 nM, 300 nM, 400 nM, 500 nM, or 1000 nM. The Kd for binding of the Fc domain to an Fc receptor in the presence of the binding domain that binds to a molecule on an immune cell, such as an antigen presenting cell, and the tumor targeting binding domain when the tumor targeting binding domain is bound to its tumor antigen can be from 100 nM to 200 nM, 100 nM to 300 nM, 100 nM to 400 nM, 100 nM to 500 nM, or 100 nM to 1000 nM. Additionally, the conjugate or antibody construct as described herein can have a Kd for binding of the Fc domain to an Fc receptor in the presence of the binding domain that binds to a molecule on an immune cell, such as an antigen presenting cell, and a tumor antigen binding domain when the tumor antigen binding domain is not bound to the tumor antigen is no greater than about 100 nM and is no greater than about 100 times a Kd for binding of the Fc domain to the Fc receptor in an absence of the binding domain that binds to a molecule on an immune cell, such as an antigen presenting cell, and a tumor antigen binding domain.

The Kd for binding of the binding domain that binds to a molecule on an immune cell, such as an antigen presenting cell, of a conjugate or antibody construct as described herein can increase when the tumor antigen binding domain is bound to its tumor antigen as compared to the Kd for binding of the binding domain that binds to a molecule on an immune cell, such as an antigen presenting cell, of a conjugate or antibody construct as described herein when the tumor antigen binding domain is not bound to its tumor antigen. For example, a conjugate or antibody construct as described herein can comprise a Kd for binding of the binding domain that binds to a molecule on an immune cell, such as an antigen presenting cell, when the tumor antigen binding domain is bound to its tumor antigen can be greater than or greater than about 100 nM. The Kd for binding of the binding domain that binds to a molecule on an immune cell, such as an antigen presenting cell, when the tumor antigen binding domain is bound to its tumor antigen can be or can be about 100 nM, 200 nM, 300 nM, 400 nM, 500 nM, or 1000 nM. Kd for binding of the binding domain that binds to a molecule on an immune cell, such as an antigen presenting cell, when the tumor antigen binding domain is bound to its tumor antigen can be from 100 nM to 200 nM, 100 nM to 300 nM, 100 nM to 400 nM, 100 nM to 500 nM, or 100 nM to 1000 nM.

The effect of the tumor antigen binding domain and the binding domain that binds to a molecule on the immune cell, such as an antigen presenting cell, together can be to cluster the conjugates or antibody constructs on cells expressing tumor antigen, and thus clustering immune cells such as an antigen presenting cells around cancerous cells and at tumor sites resulting in activation of the immune cell effector functions or antigen presenting cell effector functions. This can include the activation of the molecule on the immune cell, such as an antigen presenting cell, when a bispecific tumor targeting antibody construct or conjugate is bound to its tumor antigen, such as activation of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, or CD47. This can include the activation of the molecule on the immune cell, such as an antigen presenting cell, when a bispecific tumor targeting antibody construct or conjugate is bound to its tumor antigen, such as activation of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, CD47, TNFR2, or TREM2. In some embodiments, this activation of the molecule on the immune cell, such as an antigen presenting cell, only occurs when the bispecific tumor targeting antibody construct or conjugate is bound to its tumor antigen. An immune cell effector function or antigen presenting cell effector function can include antibody dependent cellular cytotoxicity (ADCC) of the tumor antigen expressing cell, which can occur when the bispecific tumor targeting conjugate is bound to its tumor antigen. In some embodiments, ADCC of the tumor antigen expressing cell only occurs with the bispecific tumor targeting antibody construct or conjugate is bound to its tumor antigen. An immune cell effector function or antigen presenting cell effector function can include antibody dependent cellular phagocytosis (ADCP) of the tumor antigen expressing cell, which can occur when the bispecific tumor targeting conjugate is bound to its tumor antigen. In some embodiments, ADCP of the tumor antigen expressing cell only occurs with the bispecific tumor targeting antibody construct or conjugate is bound to its tumor antigen. In certain embodiments, a bispecific tumor targeting antibody construct or conjugate density of greater than 1000, 2000, 3000, 4000, 5000, 6000, 7000, 8000, 9000, 10,000 or more per cell, resulting from the bispecific tumor targeting antibody construct or conjugate binding to the tumor antigen, induces signaling in the immune cell such as an antigen presenting cell. Signaling can suitably be measured in vitro using a cell line expressing the tumor antigen bound by the target antigen binding domain, and primary antigen presenting cells or other immune cells isolated from a human subject. Signaling can be assessed as cytokine release, chemokine release, or increased expression of cell surface markers. Cytokine release can be measured by a cytokine release assay. Chemokine release can be measured by an ELISA immunoassay. Expression of cell surface markers can be measured by Fluorescent-Activated Cell Sorting (FACS). In certain embodiments, a bispecific tumor targeting conjugate density of greater than 1000, 2000, 3000, 4000, 5000, 6000, 7000, 8000, 9000, 10,000 or more per cell, resulting from the bispecific tumor targeting antibody construct or conjugate binding to the tumor antigen, induces ADCC of the cells expressing tumor antigen. ADCC can suitably be measured in vitro using a cell line expressing the tumor antigen bound by the target antigen binding domain, and cells such as NK cells and/or macrophages isolated from a human subject. ADCC can be determined by the frequency of remaining tumor antigen expressing cells in the co-culture. ADCP can be measured by an ADCP assay, which can be determined by the frequency of remaining tumor antigen expressing cells in the co-culture.

In some embodiments, the bispecific tumor targeting antibody constructs or conjugates as described herein can specifically bind to a tumor antigen in a cluster of bispecific tumor targeting antibody constructs or conjugates, and this clustering can induce a signal in an immune cell such as an antigen presenting cell. The bispecific tumor targeting antibody constructs or conjugates as described herein can specifically bind to a tumor antigen in a cluster of bispecific tumor targeting antibody constructs or conjugates, and this clustering can induce antibody dependent cellular cytotoxicity. The bispecific tumor targeting antibody constructs or conjugates as described herein can specifically bind to a tumor antigen in a cluster of bispecific tumor targeting antibody constructs or conjugates and this clustering can result in an increased avidity for a molecule on an immune cell such as an antigen presenting cell. The bispecific tumor targeting antibody constructs or conjugates as described herein can specifically bind to a tumor antigen in a cluster of bispecific tumor targeting antibody constructs or conjugates and this clustering can result in an increased avidity of the Fc domain for an Fc receptor.

Sequences that can be used to produce antibodies for the antibody constructs and conjugates can comprise leader sequences. Leader sequences can be signal sequences. Leader sequences useful with the antibody constructs and conjugates and methods described herein can include, but are not limited to, an amino acid sequence comprising SEQ ID NO: 847, SEQ ID NO: 848, and SEQ ID NO: 849.

A binding domain of a antibody construct or conjugate can be selected in order to recognize an antigen or molecule. For example, an antigen can be a cell surface marker on target cells associated with a disease or condition. An antigen can be expressed on an immune cell. An antigen can be a peptide or fragment thereof. An antigen can be expressed on an antigen presenting cell. An antigen can be expressed on a T cell, NK cell, NKT cell, dendritic cell, a macrophage, or a B cell. An antigen on an immune cell, such as an antigen presenting cell, can be a cell lineage marker or a cell surface protein expressed preferentially on immune cells such as an antigen presenting cells or a subset of immune or antigen presenting cells. An antigen can be a peptide presented in a major histocompatibility complex by cell. As another example, a cell surface marker recognized by the antigen binding domain can include macromolecules associated with viral and bacterial diseases or infections, autoimmune diseases and cancerous diseases. An antigen can be CD40 and an antigen binding domain can recognize a CD40 antigen.

An antigen can be a tumor antigen or fragment thereof. A tumor antigen can be GD2, GD3, GM2, Ley, polysialic acid, fucosyl GM1, GM3, Tn, STn, sLe(animal), or GloboH. A tumor antigen can be any antigen listed on tumor antigen databases, such as TANTIGEN, or peptide databases for T cell-defined tumor antigens, such as the Cancer Immunity Peptide database. A tumor antigen can also be any antigen listed in the review by Chen (Chen, Cancer Immun 2004 [updated 2004 Mar. 10; cited 2004 Apr. 1]). Note that the ‘antibody’ can recognize the ‘tumor antigen’ or a peptide derived thereof, bound to an MHC molecule. An antigen can be or can be at least 80% homologous to an amino acid sequence of CD5, CD19, CD20, CD25, CD37, CD30, CD33, CD45, CAMPATH-1, BCMA, CS-1, PD-L1, B7-H3, B7-DC, HLA-DR, carcinoembryonic antigen (CEA), TAG-72, EpCAM, MUC1, folate-binding protein, A33, G250, prostate-specific membrane antigen (PSMA), ferritin, CA-125, CA19-9, epidermal growth factor, p185HER2, IL-2 receptor, EGFRvIII (de2-7), EGFR, fibroblast activation protein, tenascin, a metalloproteinase, endosialin, vascular endothelial growth factor, αvβ3, WT1, LMP2, HPV E6, HPV E7, Her-2/neu, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin, PSCA, MAGE A1, MAGE A3, CYP1B1, PLAV1, BORIS, ETV6-AML, NY-BR-1, RGS5, SART3, Carbonic anhydrase IX, PAX5, OY-TES1, Sperm protein 17, LCK, HMWMAA, AKAP-4, SSX2, XAGE 1, B7H3, Legumain, Tie 3, PAGE4, VEGFR2, MAD-CT-1, PDGFR-B, MAD-CT-2, ROR2, TRAIL1, MUC16, MAGE A4, MAGE C2, GAGE, EGFR, CMET, HER3, MUC1, CA6, NAPI2B, TROP2, CLDN6, CLDN16, CLDN18.2, RON, LY6E, FRA, DLL3, PTK7, UPK1B, VTCN1 (B7-H4), STRA6, TMPRSS3, TMRRSS4, TMEM238, C1orf186, LIV1, ROR1, Fos-related antigen 1, VEGFR1, endoglin, VISTA, LLRC15, or a fragment thereof. An antigen binding domain can be capable of recognizing a single antigen. An antigen binding domain can be capable of recognizing two or more different antigens.

An antibody construct or conjugate can comprise an Fc region with an Fc domain. An Fc domain is a structure that can bind to Fc receptors. An antibody construct or conjugate can comprise an Fc domain. Fc domains can be bound by Fc receptors (FcRs). Fc domains can be from antibodies. An Fc domain can be at least 80% homologous to an Fc domain from an antibody. An Fc region can be in a scaffold. An Fc region with an Fc domain can be in an antibody scaffold. An Fc region with an Fc domain can be in a non-antibody scaffold. An antibody construct or conjugate can comprise an Fc region with an Fc domain in an antibody scaffold. An antibody construct or conjugate can comprise an Fc region with an Fc domain in a non-antibody scaffold. An Fc domain can be in a scaffold. An Fc domain can be in an antibody scaffold. An Fc domain can be in a non-antibody scaffold. An antibody construct or conjugate can comprise an Fc domain in an antibody scaffold. An antibody construct or conjugate can comprise an Fc domain in a non-antibody scaffold. Fc domains of antibodies, including those of the present disclosure, can be bound by Fc receptors (FcRs). Fc domains can be a portion of the Fc region of an antibody. FcRs can bind to an Fc domain of an antibody. FcRs can bind to an Fc domain of an antibody bound to an antigen. FcRs can be organized into classes (e.g., gamma (γ), alpha (α) and epsilon (ε)) based on the class of antibody that the FcR recognizes. The FcαR class can bind to IgA and includes several isoforms, FcαRI (CD89) and FcαμR. The FcγR class can bind to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant. An FcγRIIIA (CD16a) can be an FcγRIIIA (CD16a) V158 variant. Each FcγR isoform can differ in affinity to the Fc region of the IgG antibody. For example, FcγRI can bind to IgG with greater affinity than FcγRII or FcγRIII. The affinity of a particular FcγR isoform to IgG can be controlled, in part, by a glycan (e.g., oligosacccharaide) at position CH2 84.4 of the IgG antibody. For example, fucose containing CH2 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc region binding to an FcR can lead to the maturation of immune cells. FcR-mediated signaling that can result from an Fc domain binding to an FcR can lead to the maturation of dendritic cells. FcR-mediated signaling that can result from an Fc domain binding to an FcR can lead to antibody dependent cellular cytotoxicity. FcR-mediated signaling that can result from an Fc domain binding to an FcR can lead to more efficient immune cell antigen uptake and processing. FcR-mediated signaling that can result from an Fc region binding to an FcR can lead to more efficient dendritic cell antigen uptake and processing. FcR-mediated signaling that can result from an Fc region binding to an FcR can increase antigen presentation. FcR-mediated signaling that can result from an Fc region binding to an FcR can increase antigen presentation by immune cells. FcR-mediated signaling that can result from an Fc region binding to an FcR can increase antigen presentation by antigen presenting cells. FcR-mediated signaling that can result from an Fc domain binding to an FcR can increase antigen presentation by dendritic cells. FcR-mediated signaling that can result from an Fc domain binding to an FcR can promote the expansion and activation of T cells. FcR-mediated signaling that can result from an Fc domain binding to an FcR can promote the expansion and activation of CD8+ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a TH1 cell response).

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

A modification in the amino acid sequence Fc domain can alter the recognition of an FcR for the Fc domain. However, such modifications can still allow for FcR-mediated signaling. A modification can be a substitution of an amino acid at a residue (e.g., wildtype) for a different amino acid at that residue. A modification can permit binding of an FcR to a site on the Fc region that the FcR may not otherwise bind to. A modification can increase binding affinity of an FcR to the Fc domain that the FcR may have reduced binding affinity for. A modification can decrease binding affinity of an FcR to a site on the Fc domain that the FcR may have increased binding affinity for. A modification can increase the subsequent FcR-mediated signaling after Fc binding to an FcR.

An antibody construct or conjugate can comprise an Fc region with at least one amino acid change as compared to the sequence of the wild-type Fc region. An antibody construct or conjugate can comprise an Fc domain with at least one amino acid change as compared to the sequence of the wild-type Fc domain. An amino acid change in an Fc region can allow the antibody construct or conjugate to bind to at least one Fc receptor with greater affinity compared to a wild-type Fc region. An amino acid change in an Fc domain can allow the antibody to bind to at least one Fc receptor with greater affinity compared to a wild-type Fc domain. An Fc region can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc region can be an Fc region of an anti-CD40 antibody. An Fc domain can be an Fc domain of an anti-CD40 antibody. An Fc region can contain an Fc domain. An Fc region can be an Fc domain.

An antibody construct or conjugate can comprise an antibody comprising a sequence of the IgG isoform that has been modified from the wildtype IgG sequence. A modification can comprise a substitution at one or more one amino acid residues of an Fc domain such as at 5 different amino acid residues including L235V/F243L/R292P/Y300L/P396L (IgG1VLPLL). The numbering of amino acids residues described herein can be according to the EU index. This modification can be located in a portion of an antibody sequence which can encode an Fc region of the antibody and in particular, can be located in portions of the Fc region that can bind to Fc receptors (i.e., the Fc domain). A modification can comprise a substitution at one or more amino acid residues such as at 2 different amino acid residues of an Fc domain including S239D/I332E (IgG1DE). This modification can be located in a portion of an antibody sequence which encodes an Fc region of the antibody and in particular, are located in portions of the Fc region that can bind to Fc receptors (i.e., the Fc domain). A modification can comprise a substitution at one or more amino acid residues such as at 3 different amino acid residues of an Fc domain including S298A/E333A/K334A (IgG1AAA). The modification can be located in a portion of an antibody sequence which can encode an Fc region of the antibody and in particular, can be located in portions of the Fc region that can bind Fc receptors (i.e., the Fc domain).

An antibody construct or conjugate can comprise a monoclonal anti-CD40 human antibody comprising a sequence of the IgG isoform that has been modified from the wildtype IgG1 sequence. A modification can comprise a substitution at one or more one amino acid residues such as at 5 different amino acid residues of an Fc domain including L235V/F243L/R292P/Y300L/P396L (SBT-040-G1VLPLL). The numbering of amino acids residues described herein can be according to the EU index. This modification can be located in a portion of an antibody sequence which can encode an Fc region of the antibody and in particular, can be located in portions of the Fc region that can bind to Fc receptors (i.e., the Fc domain). A modification can comprise a substitution at one or more amino acid residues such as at 2 different amino acid residues of an Fc domain including S239D/I332E (SBT-040-G1DE). This modification can be located in a portion of an antibody sequence which encodes an Fc region of the antibody and in particular, are located in portions of the Fc region that can bind to Fc receptors (i.e., the Fc domain). A modification can comprise a substitution at one or more one amino acid residues such as at 3 different amino acid residues of an Fc domain including S298A/E333A/K334A (SBT-040-G1AAA). This modification can be located in a portion of an antibody sequence which can encode an Fc region of the antibody and in particular, can be located in portions of the Fc region that can bind Fc receptors (i.e., the Fc domain).

Binding of Fc receptors to an Fc region can be affected by amino acid substitutions. For example, SBT-040-VLPLL is an antibody with an amino acid sequence of a heavy chain of human anti-CD40 monoclonal antibody with modifications to a wild-type IgG Fc domain (L235V/F243L/R292P/Y300L/P396L). Binding of some Fc receptors to the Fc region of SBT-040-VLPLL can be enhanced compared to wild-type by as result of the L235V/F243L/R292P/Y300L/P396L amino acid modifications. However, binding of other Fc receptors to the Fc region of SBT-040-VLPLL can be reduced compared to wild-type by the L235V/F243L/R292P/Y300L/P396L amino acid modifications. For example, the binding affinities of SBT-040-VLPLL to FcγRIIIA and to FcγRIIA can be enhanced compared to wild-type whereas the binding affinity of SBT-040-VLPLL to FcγRIIB can be reduced compared to wild-type. SBT-040-DE antibody is an antibody with an amino acid sequence of a heavy chain of human anti-CD40 monoclonal antibody with modifications to a wild-type IgG1 Fc domain (S239D/I332E). Binding of Fc receptors to the Fc region of SBT-040-DE can be enhanced compared to wild-type as a result of the S239D/I332E amino acid modification. However, binding of some Fc receptors to the Fc region of SBT-040-G1DE can be reduced compared to wild-type by S239D/I332E amino acid modification. For example, the binding affinities of SBT-040-DE to FcγRIIIA and to FcγRIIB can be enhanced compared to wild-type. Binding of Fc receptors to an Fc region of are affected by amino acid substitutions. SBT-040-G1AAA antibody is an antibody with an amino acid sequence of a heavy chain of a human anti-CD40 monoclonal antibody with modifications to a wild-type IgG1 Fc domain (S298A/E333A/K334A). Binding of Fc receptors to an Fc region of SBT-040-G1AAA can be enhanced compared to wild-type as a result of the S298A/E333A/K334A amino acid modification. However, binding of some Fc receptors to the Fc region of SBT-040-G1AAA can be reduced compared to wild-type by S298A/E333A/K334A amino acid modification. Binding affinities of SBT-040-G1AAA to FcγRIIIA can be enhanced compared to wild-type whereas the binding affinity of SBT-040-G1AAA to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

While an antibody construct or conjugate of the present disclosure can comprise a first binding domain and a second binding domain (or, in some cases, a third binding domain) with wild-type or modified amino acid sequences encoding the Fc region or Fc domain, the modifications of the Fc region or the Fc domain from the wild-type sequence may not significantly alter binding and/or affinity of the binding domains. For example, binding and/or affinity of an antibody construct or conjugate comprising a first binding domain and a second binding domain (or, in some cases, a third binding domain) and having the Fc domain modifications of SBT-040-G1WT, SBT-040-G1VLPLL, SBT-040-G1DE, or SBT-040-G1AAA may not be significantly altered by modification of an Fc region or Fc domain amino acid sequence compared to a wild-type sequence. Modifications of an Fc region or Fc domain from a wild-type sequence may not alter binding and/or affinity of a first binding domain that binds, for example, to CD40 or DEC-205. Additionally, the binding and/or affinity of the binding domains described herein, for example a first binding domain, a second binding domain (or, in some cases, a third binding domain), and an Fc domain modification selected from SBT-040-G1WT, SBT-040-G1VLPLL, SBT-040-G1DE, and SBT-040-G1AAA, may be comparable to the binding and/or affinity of wild-type antibodies.

In some embodiments, a K_{d} for binding of the first binding domain to the tumor antigen in the presence of the immune-stimulatory compound is no greater than about two times, five times, ten times, or fifty times a K_{d} for binding of the first binding domain to the tumor antigen in an absence of the immune-stimulatory compound. In some embodiments, a Kd for binding of the second binding domain to the antigen on the antigen presenting cell in the presence of the immune-stimulatory compound is no greater than about two times, five times, ten times, or fifty times a Kd for binding of the second binding domain to the antigen on the antigen presenting cell in an absence of the immune-stimulatory compound. In some embodiments, a Kd for binding of the first binding domain to the tumor antigen is no greater than about 100 nM. In some embodiments, a Kd for binding of the second binding domain to the antigen on an antigen presenting cell is no greater than about 100 nM.

In some embodiments, a Kd for binding of the Fc domain to the Fc receptor in the presence of the immune-stimulatory compound is no greater than about two times, five times, ten times, or fifty times a Kd for binding of the Fc domain to the Fc receptor in an absence of the immune-stimulatory compound.

In some embodiments, the Fc domain is an Fc domain variant comprising at least one amino acid residue change as compared to a wild type sequence of the Fc domain.

In some embodiments, the Fc domain variant binds to an Fc receptor with altered affinity as compared to the wild type Fc domain.

In some embodiments, the at least one amino acid residue change is selected from a group consisting of: a) F243L, R292P, Y300L, L235V, and P396L, wherein numbering of amino acid residues in the Fc domain is according to the EU index; b) S239D and I332E, wherein numbering of amino acid residues in the Fc domain is according to the EU index; and c) S298A, E333A, and K334A, wherein numbering of amino acid residues in the Fc domain is according to the EU index.

In some embodiments, the antibody construct or conjugate induces secretion of cytokines by an immune cell as measured by a cytokine release assay. In some embodiments, the cytokine is IFN-γ, IL-8, IL-12, IL-2, or a combination thereof. In some embodiments, the antibody construct or conjugate induces antigen presentation on a dendritic cell, B cell, macrophage, or a combination thereof.

TABLE 3
Exemplary Tumor Antibody CDRs

| Antibody | Region | SEQ ID NO: |
|---|---|---|
| Nimotuzumab | HCDR1 | 52 |
| | HCDR2 | 53 |
| | HCDR3 | 54 |
| | LCDR1 | 57 |
| | LCDR2 | 58 |
| | LCDR3 | 59 |
| Zalutumumab | HCDR1 | 65 |
| | HCDR2 | 66 |
| | HCDR3 | 67 |
| | LCDR1 | 70 |
| | LCDR2 | 71 |
| | LCDR3 | 72 |
| Onartuzumab | HCDR1 | 78 |
| | HCDR2 | 79 |
| | HCDR3 | 80 |
| | LCDR1 | 83 |
| | LCDR2 | 84 |
| | LCDR3 | 85 |
| Patritumab | HCDR1 | 91 |
| | HCDR2 | 92 |
| | HCDR3 | 93 |
| | LCDR1 | 96 |
| | LCDR2 | 97 |
| | LCDR3 | 98 |
| Clivatuzumab | HCDR1 | 104 |
| | HCDR2 | 105 |
| | HCDR3 | 106 |
| | LCDR1 | 109 |
| | LCDR2 | 110 |
| | LCDR3 | 111 |
| Sofituzumab | HCDR1 | 117 |
| | HCDR2 | 118 |
| | HCDR3 | 119 |
| | LCDR1 | 122 |
| | LCDR2 | 123 |
| | LCDR3 | 124 |
| Edrecolomab | HCDR1 | 130 |
| | HCDR2 | 131 |
| | HCDR3 | 132 |
| | LCDR1 | 135 |
| | LCDR2 | 136 |
| | LCDR3 | 137 |
| Adecatumumab | HCDR1 | 143 |
| | HCDR2 | 144 |
| | HCDR3 | 145 |
| | LCDR1 | 148 |
| | LCDR2 | 149 |
| | LCDR3 | 150 |
| Anetumab | HCDR1 | 156 |
| | HCDR2 | 157 |
| | HCDR3 | 158 |
| | LCDR1 | 161 |
| | LCDR2 | 162 |
| | LCDR3 | 163 |
| huDS6 | HCDR1 | 169 |
| | HCDR2 | 170 |
| | HCDR3 | 171 |
| | LCDR1 | 174 |
| | LCDR2 | 175 |
| | LCDR3 | 176 |
| Lifastuzumab | HCDR1 | 182 |
| | HCDR2 | 183 |
| | HCDR3 | 184 |
| | LCDR1 | 187 |
| | LCDR2 | 188 |
| | LCDR3 | 189 |
| Sacituzumab | HCDR1 | 195 |
| | HCDR2 | 196 |
| | HCDR3 | 197 |
| | LCDR1 | 200 |
| | LCDR2 | 201 |
| | LCDR3 | 202 |
| PR1A3 | HCDR1 | 208 |
| | HCDR2 | 209 |
| | HCDR3 | 210 |
| | LCDR1 | 213 |
| | LCDR2 | 214 |
| | LCDR3 | 215 |
| Humanized PR1A3 | HCDR1 | 805 |
| | HCDR2 | 806 |
| | HCDR3 | 807 |
| | LCDR1 | 808 |
| | LCDR2 | 809 |
| | LCDR3 | 810 |
| Humanized Ab2-3 | HCDR1 | 823 |
| | HCDR2 | 824 |
| | HCDR3 | 825 |
| | LCDR1 | 826 |
| | LCDR2 | 827 |
| | LCDR3 | 828 |
| IMAB362, | HCDR1 | 221 |
| Claudiximab | HCDR2 | 222 |
| | HCDR3 | 223 |
| | LCDR1 | 226 |
| | LCDR2 | 227 |
| | LCDR3 | 228 |
| AMG595 | HCDR1 | 260 |
| | HCDR2 | 261 |
| | HCDR3 | 262 |
| | LCDR1 | 265 |
| | LCDR2 | 266 |
| | LCDR3 | 267 |
| ABT806 | HCDR1 | 273 |
| | HCDR2 | 274 |
| | HCDR3 | 275 |
| | LCDR1 | 278 |
| | LCDR2 | 279 |
| | LCDR3 | 280 |
| Sibrotuzumab | HCDR1 | 286 |
| | HCDR2 | 287 |
| | HCDR3 | 288 |
| | LCDR1 | 291 |
| | LCDR2 | 292 |
| | LCDR3 | 293 |
| DS-8895a variant 1 | HCDR1 | 299 |
| | HCDR2 | 300 |
| | HCDR3 | 301 |
| | LCDR1 | 304 |
| | LCDR2 | 305 |
| | LCDR3 | 306 |
| DS-8895a variant 2 | HCDR1 | 312 |
| | HCDR2 | 313 |
| | HCDR3 | 314 |
| | LCDR1 | 317 |
| | LCDR2 | 318 |
| | LCDR3 | 319 |
| Anti-EphA2 | HCDR1 | 325 |
| | HCDR2 | 326 |
| | HCDR3 | 327 |
| | LCDR1 | 330 |
| | LCDR2 | 331 |
| | LCDR3 | 332 |
| Narnatumab | HCDR1 | 338 |
| | HCDR2 | 339 |
| | HCDR3 | 340 |
| | LCDR1 | 343 |
| | LCDR2 | 344 |
| | LCDR3 | 345 |
| RG7841 | HCDR1 | 351 |
| | HCDR2 | 352 |
| | HCDR3 | 353 |
| | LCDR1 | 356 |
| | LCDR2 | 357 |
| | LCDR3 | 358 |
| Farletuzumab | HCDR1 | 364 |
| | HCDR2 | 365 |
| | HCDR3 | 366 |
| | LCDR1 | 369 |
| | LCDR2 | 370 |
| | LCDR3 | 371 |
| Mirvetuximab | HCDR1 | 377 |
| | HCDR2 | 378 |
| | HCDR3 | 379 |
| | LCDR1 | 382 |
| | LCDR2 | 383 |
| | LCDR3 | 384 |
| J591 variant 1 | HCDR1 | 390 |
| | HCDR2 | 391 |
| | HCDR3 | 392 |
| | LCDR1 | 395 |
| | LCDR2 | 396 |
| | LCDR3 | 397 |
| J591 variant 2 | HCDR1 | 403 |
| | HCDR2 | 404 |
| | HCDR3 | 405 |
| | LCDR1 | 408 |
| | LCDR2 | 409 |
| | LCDR3 | 410 |
| Rovalpituzumab | HCDR1 | 416 |
| | HCDR2 | 417 |
| | HCDR3 | 418 |
| | LCDR1 | 421 |
| | LCDR2 | 422 |
| | LCDR3 | 423 |
| PF-06647020 | HCDR1 | 429 |
| | HCDR2 | 430 |
| | HCDR3 | 431 |
| | LCDR1 | 434 |
| | LCDR2 | 435 |
| | LCDR3 | 436 |
| Anti-PTK7 | HCDR1 | 442 |
| | HCDR2 | 443 |
| | HCDR3 | 444 |
| | LCDR1 | 447 |
| | LCDR2 | 448 |
| | LCDR3 | 449 |
| Ladiratuzumab | HCDR1 | 455 |
| | HCDR2 | 456 |
| | HCDR3 | 457 |
| | LCDR1 | 460 |
| | LCDR2 | 461 |
| | LCDR3 | 462 |
| Cirmtuzumab | HCDR1 | 468 |
| | HCDR2 | 469 |
| | HCDR3 | 470 |
| | LCDR1 | 473 |
| | LCDR2 | 474 |
| | LCDR3 | 475 |
| anti-MAGE-A3 | HCDR1 | 481 |
| | HCDR2 | 482 |
| | HCDR3 | 483 |
| | LCDR1 | 486 |
| | LCDR2 | 487 |
| | LCDR3 | 488 |
| Anti-NY-ESO-1 | HCDR1 | 494 |
| | HCDR2 | 495 |
| | HCDR3 | 496 |
| | LCDR1 | 499 |
| | LCDR2 | 500 |
| | LCDR3 | 501 |

TABLE 4
Additional Exemplary Tumor Antibody CDRs

| Antibody | Region | SEQ ID NO: |
|---|---|---|
| Pertuzumab | HCDR1 | 13 |
| | HCDR2 | 14 |
| | HCDR3 | 15 |
| | LCDR1 | 18 |
| | LCDR2 | 19 |
| | LCDR3 | 20 |
| Trastuzumab | HCDR1 | 673 |
| | HCDR2 | 674 |
| | HCDR3 | 675 |
| | LCDR1 | 676 |
| | LCDR2 | 677 |
| | LCDR3 | 678 |
| Cetuximab | HCDR1 | 26 |
| | HCDR2 | 27 |
| | HCDR3 | 28 |
| | LCDR1 | 31 |
| | LCDR2 | 32 |
| | LCDR3 | 33 |
| Panitumumab | HCDR1 | 39 |
| | HCDR2 | 40 |
| | HCDR3 | 41 |
| | LCDR1 | 44 |
| | LCDR2 | 45 |
| | LCDR3 | 46 |

TABLE 5
Tumor Antibody V_{H} sequences and V_{L} sequences

| Antibody | Region | SEQ ID NO: |
|---|---|---|
| Nimotuzumab | V_{H} | 51 |
| | V_{L} | 56 |
| Zalutumumab | V_{H} | 64 |
| | V_{L} | 69 |
| Onartuzumab | V_{H} | 77 |
| | V_{L} | 82 |
| Patritumab | V_{H} | 90 |
| | V_{L} | 95 |
| Clivatuzumab | V_{H} | 103 |
| | V_{L} | 108 |
| Sofituzumab | V_{H} | 116 |
| | V_{L} | 121 |
| Edrecolomab | V_{H} | 129 |
| | V_{L} | 134 |
| Adecatumumab | V_{H} | 142 |
| | V_{L} | 147 |
| Anetumab | V_{H} | 155 |
| | V_{L} | 160 |
| huDS6 | V_{H} | 168 |
| | V_{L} | 173 |
| Lifastuzumab | V_{H} | 181 |
| | V_{L} | 186 |
| Sacituzumab | V_{H} | 194 |
| | V_{L} | 199 |
| PR1A3 | V_{H} | 207 |
| | V_{L} | 212 |
| Humanized PR1A3 | V_{H} | 811 |
| | V_{L} | 812 |
| Humanized Ab2-3 | V_{H} | 829 |
| | V_{L} | 830 |
| IMAB362, | V_{H} | 220 |
| Claudiximab | V_{L} | 225 |
| AMG595 | V_{H} | 259 |
| | V_{L} | 264 |
| ABT806 | V_{H} | 272 |
| | V_{L} | 277 |
| Sibrotuzumab | V_{H} | 285 |
| | V_{L} | 290 |
| DS-8895a variant 1 | V_{H} | 298 |
| | V_{L} | 303 |
| DS-8895a variant 2 | V_{H} | 311 |
| | V_{L} | 316 |
| Anti-EphA2 | V_{H} | 324 |
| | V_{L} | 329 |
| Narnatumab | V_{H} | 337 |
| | V_{L} | 342 |
| RG7841 | V_{H} | 350 |
| | V_{L} | 355 |
| Farletuzumab | V_{H} | 363 |
| | V_{L} | 368 |
| Mirvetuximab | V_{H} | 376 |
| | V_{L} | 381 |
| J591 variant 1 | V_{H} | 389 |
| | V_{L} | 394 |
| J591 variant 2 | V_{H} | 402 |
| | V_{L} | 407 |
| Rovalpituzumab | V_{H} | 415 |
| | V_{L} | 420 |
| PF-06647020 | V_{H} | 428 |
| | V_{L} | 433 |
| Anti-PTK7 | V_{H} | 441 |
| | V_{L} | 446 |
| Ladiratuzumab | V_{H} | 454 |
| | V_{L} | 459 |
| Cirmtuzumab | V_{H} | 467 |
| | V_{L} | 472 |
| Anti-MAGE-A3 | V_{H} | 480 |
| | V_{L} | 485 |
| Anti-NY-ESO-1 | V_{H} | 493 |
| | V_{L} | 498 |

TABLE 6
Additional Exemplary Tumor Antibody
V_{H} sequences and V_{L} sequences

| Antibody | Region | SEQ ID NO: |
|---|---|---|
| Pertuzumab | V_{H} | 12 |
| | V_{L} | 17 |
| Cetuximab | V_{H} | 25 |
| | V_{L} | 30 |
| Panitumumab | V_{H} | 38 |
| | V_{L} | 43 |
| | V_{L} | 498 |
| Trastuzumab | V_{H} | 679 |
| | V_{L} | 680 |

TABLE 7
Exemplary Tumor Antibody Heavy Chain
(HC) and Light Chain (LC) sequences

| Antibody | Region | SEQ ID NO: |
|---|---|---|
| Nimotuzumab | HC | 50 |
| | LC | 55 |
| Zalutumumab | HC | 63 |
| | LC | 68 |
| Onartuzumab | HC | 76 |
| | LC | 81 |
| Patritumab | HC | 89 |
| | LC | 94 |
| Clivatuzumab | HC | 102 |
| | LC | 107 |
| Sofituzumab | HC | 115 |
| | LC | 120 |
| Edrecolomab | HC | 128 |
| | LC | 133 |
| Adecatumumab | HC | 141 |
| | LC | 146 |
| Anetumab | HC | 154 |
| | LC | 159 |
| huDS6 | HC | 167 |
| | LC | 172 |
| Lifastuzumab | HC | 180 |
| | LC | 185 |
| Sacituzumab | HC | 193 |
| | LC | 198 |
| PR1A3 | HC | 206 |
| | LC | 211 |
| Humanized PR1A3 | HC | 813 |
| | LC | 814 |
| Humanized Ab2-3 | HC | 831 |
| | LC | 832 |
| IMAB362, | HC | 219 |
| Claudiximab | LC | 224 |
| AMG595 | HC | 258 |
| | LC | 263 |
| ABT806 | HC | 271 |
| | LC | 276 |
| Sibrotuzumab | HC | 284 |
| | LC | 289 |
| DS-8895a variant 1 | HC | 297 |
| | LC | 302 |
| DS-8895a variant 2 | HC | 310 |
| | LC | 315 |
| Anti-EphA2 | HC | 323 |
| | LC | 328 |
| Narnatumab | HC | 336 |
| | LC | 341 |
| RG7841 | HC | 349 |
| | LC | 354 |
| Farletuzumab | HC | 362 |
| | LC | 367 |
| Mirvetuximab | HC | 375 |
| | LC | 380 |
| J591 variant 1 | HC | 388 |
| | LC | 393 |
| J591 variant 2 | HC | 401 |
| | LC | 406 |
| Rovalpituzumab | HC | 414 |
| | LC | 419 |
| PF-06647020 | HC | 427 |
| | LC | 432 |
| Anti-PTK7 | HC | 440 |
| | LC | 445 |
| Ladiratuzumab | HC | 453 |
| | LC | 458 |
| Cirmtuzumab | HC | 466 |
| | LC | 471 |
| Anti-MAGE-A3 | HC | 479 |
| | LC | 484 |
| Anti-NY-ESO-1 | HC | 492 |
| | LC | 497 |
| | LC | 682 |

TABLE 8
Additional Exemplary Tumor Antibody Heavy
Chain (HC) and Light Chain (LC) sequences

| Antibody | Region | SEQ ID NO: |
|---|---|---|
| Pertuzumab | HC | 11 |
| | LC | 16 |
| Cetuximab | HC | 24 |
| | LC | 29 |
| Panitumumab | HC | 37 |
| | LC | 42 |
| | LC | 263 |
| Trastuzumab | HC | 681 |
| | LC | 682 |

TABLE 9
Exemplary Fusion Sequences - CD40 fusions via the heavy chain

| | SEQ |
|---|---|
| Fusion | ID NO: |
| HC CD40 mAb with Cetuximab ScFv | 34 |
| HC Cetuximab with CD40 mAb ScFv | 35 |
| HC CD40 mAb with Panitumumab ScFv | 47 |
| HC Panitumumab with CD40 mAb ScFv | 48 |
| HC CD40 mAb with Nimotuzumab ScFv | 60 |
| HC Nimotuzumab with CD40 mAb ScFv | 61 |
| HC CD40 mAb with Zalutumumab ScFv | 73 |
| HC Zalutumumab with CD40 mAb ScFv | 74 |
| HC CD40 mAb with Onartuzumab ScFv | 86 |
| HC Onartuzumab with CD40 mAb ScFv | 87 |
| HC CD40 mAb with Patritumab ScFv | 99 |
| HC Patritumab with CD40 mAb ScFv | 100 |
| HC CD40 mAb with Clivatuzumab ScFv | 112 |
| HC Clivatuzumab with CD40 mAb ScFv | 113 |
| HC CD40 mAb with Sofituzumab ScFv | 125 |
| HC Sofituzumab with CD40 mAb ScFv | 126 |
| HC CD40 mAb with Edrecolomab ScFv | 138 |
| HC Edrecolomab with CD40 mAb ScFv | 139 |
| HC CD40 mAb with Adecatumumab ScFv | 151 |
| HC Adecatumumab with CD40 mAb ScFv | 152 |
| HC CD40 mAb with Anetumab ScFv | 164 |
| HC Anetumab with CD40 mAb ScFv | 165 |
| HC CD40 mAb with huDS6 mAb ScFv | 177 |
| HC huDS6mAb with CD40 mAb ScFv | 178 |
| HC CD40 mAb with Lifastuzumab ScFv | 190 |
| HC Lifastuzumab with CD40 mAb ScFv | 191 |
| HC CD40 mAb with Sacituzumab ScFv | 203 |
| HC Sacituzumab with CD40 mAb ScFv | 204 |
| HC CD40 mAb with PR1A3 mAb ScFv | 216 |
| HC PR1A3mAb with CD40 mAb ScFv | 217 |
| HC CD40 mAb with Humanized PR1A3 mAb ScFv | 815 |
| HC Humanized PR1A3 mAb with CD40 mAb ScFv | 816 |
| HC Humanized PR1A3with CD40 mAb scFv (LH) | 843 |
| HC CD40 mAb with Humanized Ab2-3 mAb ScFv | 833 |
| HC Humanized Ab2-3 mAb with CD40 mAb ScFv | 834 |
| HC Humanized Ab2-3 mAb with CD40 mAb scFv (LH) | 841 |
| HC CD40 mAb with Claudiximab ScFv | 229 |
| HC Claudiximab with CD40 mAb ScFv | 230 |
| HC CD40 mAb with AMG595 mAb ScFv | 268 |
| HC AMG595 mAb with CD40 mAb ScFv | 269 |
| HC CD40 mAb with ABT806 mAb ScFv | 281 |
| HC ABT806 mAb with CD40 mAb ScFv | 282 |
| HC CD40 mAb with Sibrotuzumab mAb ScFv | 294 |
| HC Sibrotuzumab with CD40 mAb ScFv | 295 |
| HC CD40 mAb with DS-8895a variant 1 mAb ScFv | 307 |
| HC DS-8895a variant 1 mAb with CD40 mAb ScFv | 308 |
| HC CD40 mAb with DS-8895a variant 2 mAb ScFv | 320 |
| HC DS-8895a variant 2 mAb with CD40 mAb ScFv | 321 |
| HC CD40 mAb with Anti-EphA2 mAb ScFv | 333 |
| HC Anti-EphA2 mAb with CD40 mAb ScFv | 334 |
| HC CD40 mAb with Narnatumab mAb ScFv | 346 |
| HC Narnatumab mAb with CD40 mAb ScFv | 347 |
| HC CD40 mAb with RG7841 mAb ScFv | 359 |
| HC RG7841 mAb with CD40 mAb ScFv | 360 |
| HC CD40 mAb with Farletuzumab mAb ScFv | 372 |
| HC Farletuzumab mAb with CD40 mAb ScFv | 373 |
| HC CD40 mAb with Mirvetuximab mAb ScFv | 385 |
| HC Mirvetuximab mAb with CD40 mAb ScFv | 386 |
| HC CD40 mAb with J591 variant 1 mAb ScFv | 398 |
| HC J591 variant 1 mAb with CD40 mAb ScFv | 399 |
| HC CD40 mAb with J591 variant 2 mAb ScFv | 411 |
| HC J591 variant 2 mAb with CD40 mAb ScFv | 412 |
| HC CD40 mAb with Rovalpituzumab mAb ScFv | 424 |
| HC Rovalpituzumab mAb with CD40 mAb ScFv | 425 |
| HC CD40 mAb with PF-06647020 mAb ScFv | 437 |
| HC PF-06647020 mAb with CD40 mAb ScFv | 438 |
| HC CD40 mAb with anti-PTK7 mAb ScFv | 450 |
| HC PTK7 mAb with CD40 mAb ScFv | 451 |
| HC CD40 mAb with mAb of Ladiratuzumab ScFv | 463 |
| HC Ladiratuzumab with CD40 mAb ScFv | 464 |
| HC CD40 mAb with Cirmtuzumab mAb ScFv | 476 |
| HC Cirmtuzumab mAb with CD40 mAb ScFv | 477 |
| HC CD40 mAb with anti-MAGE-A3 mAb ScFv | 489 |
| HC tumor mAb with CD40 mAb ScFv | 490 |
| HC CD40 mAb with anti-NY-ESO-1 mAb ScFv | 502 |
| HC anti-NY-ESO-1 mAb with CD40 mAb ScFv | 503 |

TABLE 10
Additional Exemplary Fusion Sequences -
CD40 fusions via the heavy chain

| | SEQ |
|---|---|
| Fusion | ID NO: |
| HC CD40 mAb with Pertuzumab ScFv | 21 |
| HC Pertuzumab with CD40 mAb ScFv | 22 |
| HC Pertuzumab with CD40 mAb scFv (LH) | 845 |
| HC CD40 mAb with Trastuzumab mAb ScFv | 683 |
| HC Trastuzumab mAb with CD40 mAb ScFv | 684 |
| HC CD40 mAb with Trastuzumab mAb ScFv (LH, 25mer) | 796 |

TABLE 11
Exemplary APC Antibody CDRs

| Antibody | Region | SEQ ID NO: |
|---|---|---|
| CP-870893 | HCDR1 | 3 |
| | HCDR2 | 4 |
| | HCDR3 | 5 |
| | LCDR1 | 8 |
| | LCDR2 | 9 |
| | LCDR3 | 10 |
| DEC-205 variant 1 | HCDR1 | 234 |
| | HCDR2 | 235 |
| | HCDR3 | 236 |
| | LCDR1 | 239 |
| | LCDR2 | 240 |
| | LCDR3 | 241 |
| DEC-205 variant 2 | HCDR1 | 247 |
| | HCDR2 | 248 |
| | HCDR3 | 249 |
| | LCDR1 | 252 |
| | LCDR2 | 253 |
| | LCDR3 | 254 |
| DC-SIGN variant 1 | HCDR1 | 640 |
| | HCDR2 | 641 |
| | HCDR3 | 642 |
| | LCDR1 | 643 |
| | LCDR2 | 644 |
| | LCDR3 | 645 |
| DC-SIGN variant 2 | HCDR1 | 646 |
| | HCDR2 | 647 |
| | HCDR3 | 648 |
| | LCDR1 | 649 |
| | LCDR2 | 650 |
| | LCDR3 | 651 |
| DC-SIGN variant 3 | HCDR1 | 652 |
| | HCDR2 | 653 |
| | HCDR3 | 654 |
| | LCDR1 | 655 |
| | LCDR2 | 656 |
| | LCDR3 | 657 |

TABLE 12
Additional Exemplary APC Antibody CDRs

| Antibody | Region | SEQ ID NO: |
|---|---|---|
| SBT-040 (G1/G2) | HCDR1 | 3 |
| | HCDR2 | 4 |
| | HCDR3 | 5 |
| | LCDR1 | 8 |
| | LCDR2 | 9 |
| | LCDR3 | 10 |
| Dacetuzumab | HCDR1 | 582 |
| | HCDR2 | 583 |
| | HCDR3 | 584 |
| | LCDR1 | 587 |
| | LCDR2 | 588 |
| | LCDR3 | 589 |
| Bleselumab | HCDR1 | 592 |
| | HCDR2 | 593 |
| | HCDR3 | 594 |
| | LCDR1 | 597 |
| | LCDR2 | 598 |
| | LCDR3 | 599 |
| lucatumumab | HCDR1 | 602 |
| | HCDR2 | 603 |
| | HCDR3 | 604 |
| | LCDR1 | 607 |
| | LCDR2 | 608 |
| | LCDR3 | 609 |
| ADC-1013 | HCDR1 | 612 |
| | HCDR2 | 613 |
| | HCDR3 | 614 |
| | LCDR1 | 617 |
| | LCDR2 | 618 |
| | LCDR3 | 619 |
| APX005 | HCDR1 | 622 |
| | HCDR2 | 623 |
| | HCDR3 | 624 |
| | LCDR1 | 627 |
| | LCDR2 | 628 |
| | LCDR3 | 629 |
| Chi Lob 7/4 | HCDR1 | 632 |
| | HCDR2 | 633 |
| | HCDR3 | 634 |
| | LCDR1 | 637 |
| | LCDR2 | 638 |
| | LCDR3 | 639 |
| | HCDR2 | 653 |
| | HCDR3 | 654 |
| | LCDR1 | 655 |
| | LCDR2 | 656 |
| | LCDR3 | 657 |

TABLE 13
Exemplary APC Antibody V_{H} sequences and V_{L} sequences

| Antibody | Region | SEQ ID NO: |
|---|---|---|
| CP-870893 | V_{H} | 2 |
| | V_{L} | 7 |
| DEC-205 variant 1 | V_{H} | 233 |
| | V_{L} | 238 |
| DEC-205 variant 2 | V_{H} | 246 |
| | V_{L} | 251 |
| CD36 mannose | V_{H} | 658 |
| Scavenger Receptor | V_{L} | 659 |
| CLEC9A | V_{H} | 660 |
| | V_{L} | 661 |

TABLE 14
Additional Exemplary APC Antibody
V_{H} sequences and V_{L} sequences

| Antibody | Region | SEQ ID NO: |
|---|---|---|
| SBT-040 | V_{H} | 2 |
| | V_{L} | 7 |
| Dacetuzumab | V_{H} | 581 |
| | V_{L} | 586 |
| Bleselumab | V_{H} | 591 |
| | V_{L} | 596 |
| Lucatumumab | V_{H} | 601 |
| | V_{L} | 606 |
| ADC-1013 | V_{H} | 611 |
| | V_{L} | 616 |
| APX005 | V_{H} | 621 |
| | V_{L} | 626 |
| Chi Lob 7/4 | V_{H} | 631 |
| | V_{L} | 636 |

TABLE 15
Exemplary APC Antibody Heavy Chain and Light Chain sequences

| Antibody | Region | SEQ ID NO: |
|---|---|---|
| CP-870893 | HC | 1 |
| | LC | 6 |
| DEC-205 | HC | 232 |
| (QVQLVQvariant 1) | LC | 237 |
| DEC-205 | HC | 245 |
| (variant 2) | LC | 250 |
| CLEC12A | HC variant 1 | 662 |
| | HC variant 2 | 663 |
| | HC variant 3 | 664 |
| | LC | 665 |
| BDCA-2 | HC | 666 |
| Variant 1 | LC | 669 |
| BDCA-2 | HC | 667 |
| Variant 2 | LC | 670 |
| BDCA-2 | HC | 668 |
| Variant 3 | LC | 671 |

TABLE 16
Additional Exemplary APC Antibody Heavy
Chain and Light Chain sequences

| Antibody | Region | SEQ ID NO: |
|---|---|---|
| Anti-CD40 | HC (IgG1) | 577 |
| | HC (IgG2) | 578 |
| | LC | 579 |
| Dacetuzumab | HC | 580 |
| | LC | 585 |
| Bleselumab | HC | 590 |
| | LC | 595 |
| Lucatumumab | HC | 600 |
| | LC | 605 |
| ADC-1013 | HC | 610 |
| | LC | 615 |
| APX005 | HC | 620 |
| | LC | 625 |
| Chi Lob 7/4 | HC | 630 |
| | LC | 635 |

TABLE 17
Exemplary Fusion Sequences - DEC-
205 fusions via the heavy chain

| | SEQ |
|---|---|
| Fusion | ID NO: |
| HC DEC-205 mAb with Pertuzumab mAb ScFv | 505 |
| HC Pertuzumab with DEC-205 mAb ScFv | 506 |
| LC Pertuzumab containing CD40 mab scFv (LH) | 846 |
| HC DEC-205 mAb with Cetuximab mAb ScFv | 507 |
| HC Cetuximab with DEC-205 mAb ScFv | 508 |
| HC DEC-205 mAb with Panitumumab mAb ScFv | 509 |
| HC Panitumumab with DEC-205 mAb ScFv | 510 |
| HC DEC-205 mAb with Nimotuzumab mAb ScFv | 511 |
| HC Nimotuzumab with DEC-205 mAb ScFv | 512 |
| HC DEC-205 mAb with Zalutumumab mAb ScFv | 513 |
| HC Zalutumumab with DEC-205 mAb ScFv | 514 |
| HC DEC-205 mAb with Onartuzumab mAb ScFv | 515 |
| HC Onartuzumab with DEC-205 mAb ScFv | 516 |
| HC DEC-205 mAb with Patritumab ScFv | 517 |
| HC Patritumab with DEC-205 mAb ScFv | 518 |
| HC DEC-205 mAb with Clivatuzumab ScFv | 519 |
| HC Clivatuzumab with DEC-205 mAb ScFv | 520 |
| HC DEC-205 mAb with Sofituzumab ScFv | 521 |
| HC Sofituzumab with DEC-205 mAb ScFv | 522 |
| HC DEC-205 mAb with Edrecolomab ScFv | 523 |
| HC Edrecolomab with DEC-205 mAb ScFv | 524 |
| HC DEC-205 mAb with Adecatumumab ScFv | 525 |
| HC Adecatumumab with DEC-205 mAb ScFv | 526 |
| HC DEC-205 mAb with Anetumab ScFv | 527 |
| HC Anetumab with DEC-205 mAb ScFv | 528 |
| HC DEC-205 mAb with huDS6 mAb ScFv | 529 |
| HC huDS6 mAb with DEC-205 mAb ScFv | 530 |
| HC DEC-205 mAb with Lifastuzumab ScFv | 531 |
| HC Lifastuzumab with DEC-205 mAb ScFv | 532 |
| HC DEC-205 mAb with Sacituzumab ScFv | 533 |
| HC Sacituzumab with DEC-205 mAb ScFv | 534 |
| HC DEC-205 mAb with PR1A3 mAb ScFv | 535 |
| HC PR1A3 mAb with DEC-205 mAb ScFv | 536 |
| HC DEC-205 mAb with Humanized PR1A3 mAb ScFv | 818 |
| HC Humanized PR1A3 mAb with DEC-205 mAb ScFv | 819 |
| HC DEC-205 mAb with Humanized Ab2-3 mAb ScFv | 836 |
| HC Humanized Ab2-3 mAb with DEC-205 mAb ScFv | 837 |
| HC DEC-205 mAb with Claudiximab ScFv | 537 |
| HC Claudiximab with DEC-205 mAb ScFv | 538 |
| HC DEC-205 mAb with AMG595 mAb ScFv | 539 |
| HC AMG595 mAb with DEC-205 mAb ScFv | 540 |
| HC DEC-205 mAb with ABT806 mAb ScFv | 541 |
| HC ABT806 mAb with DEC-205 mAb ScFv | 542 |
| HC DEC-205 mAb with Sibrotuzumab ScFv | 543 |
| HC Sibrotuzumab with DEC-205 mAb ScFv | 544 |
| HC DEC-205 mAb with DS-8895a variant 1 mAb ScFv | 545 |
| HC DS-8895a variant 1 with DEC-205 mAb ScFv | 546 |
| HC DEC-205 mAb with DS-8895a variant 2 mAb ScFv | 547 |
| HC DS-8895a variant 2 mAb with DEC-205 mAb ScFv | 548 |
| HC DEC-205 mAb with Anti-EphA2 mAb ScFv | 549 |
| HC Anti-EphA2 mAb with DEC-205 mAb ScFv | 550 |
| HC DEC-205 mAb with Narnatumab mAb ScFv | 551 |
| HC Narnatumab with DEC-205 mAb ScFv | 552 |
| HC DEC-205 mAb with RG7841 mAb ScFv | 553 |
| HC RG7841 mAb with DEC-205 mAb ScFv | 554 |
| HC DEC-205 mAb with Farletuzumab ScFv | 555 |
| HC Farletuzumab with DEC-205 mAb ScFv | 556 |
| HC DEC-205 mAb with Mirvetuximab ScFv | 557 |
| HC Mirvetuximab with DEC-205 mAb ScFv | 558 |
| HC DEC-205 mAb with J591 variant 1 mAb ScFv | 559 |
| HC J591 variant 1 mAb with DEC-205 mAb ScFv | 560 |
| HC DEC-205 mAb with J591 variant 2 mAb ScFv | 561 |
| HC J591 variant 2 mAb with DEC-205 mAb ScFv | 562 |
| HC DEC-205 mAb with Rovalpituzumab mAb ScFv | 563 |
| HC Rovalpituzumab with DEC-205 mAb ScFv | 564 |
| HC DEC-205 mAb with PF-06647020 mAb ScFv | 565 |
| HC PF-06647020 mAb with DEC-205 mAb ScFv | 566 |
| HC DEC-205 mAb with anti-PTK7 mAb ScFv | 567 |
| HC anti-PTK7 mAb with DEC-205 mAb ScFv | 568 |
| HC DEC-205 mAb with Ladiratuzumab ScFv | 569 |
| HC Ladiratuzumab with DEC-205 mAb ScFv | 570 |
| HC DEC-205 mAb with Cirmtuzumab ScFv | 571 |
| HC Cirmtuzumab with DEC-205 mAb ScFv | 572 |
| HC DEC-205 mAb with anti-MAGE-A3 mAb ScFv | 573 |
| HC anti-MAGE-A3 mAb with DEC-205 mAb ScFv | 574 |
| HC DEC-205 mAb with anti-NY-ESO-1 mAb ScFv | 575 |
| HC anti-NY-ESO-1 mAb with DEC-205 mAb ScFv | 576 |
| HC DEC-205 mAb with Trastuzumab ScFv | 686 |
| HC Trastuzumab with DEC-205 mAb ScFv | 687 |
| HC DEC205 mAb with Trastuzumab ScFv (LH, 25mer) | 797 |

TABLE 18
Exemplary Fusion Sequences - CD40 fusions via the light chain

| | SEQ |
|---|---|
| Fusion | ID NO: |
| LC CD40 mAb containing Pertuzumab ScFv | 766 |
| LC Pertuzumab containing CD40 mAb ScFv | 23 |
| LC CD40 mAb containing Cetuximab ScFv | 703 |
| LC Cetuximab containing CD40 mAb ScFv | 36 |
| LC CD40 mAb containing Panitumumab ScFv | 760 |
| LC Panitumumab containing CD40 mAb ScFv | 49 |
| LC CD40 mAb containing Nimotuzumab ScFv | 751 |
| LC Nimotuzumab containing CD40 mAb ScFv | 62 |
| LC CD40 mAb containing Zalutumumab ScFv | 802 |
| LC Zalutumumab containing CD40 mAb ScFv | 75 |
| LC CD40 mAb containing Onartuzumab ScFv | 757 |
| LC Onartuzumab containing CD40 mAb ScFv | 88 |
| LC CD40 mAb containing Patritumab ScFv | 763 |
| LC Patritumab containing CD40 mAb ScFv | 101 |
| LC CD40 mAb containing Clivatuzumab ScFv | 709 |
| LC Clivatuzumab containing CD40 mAb ScFv | 114 |
| LC CD40 mAb containing Sofituzumab ScFv | 790 |
| LC Sofituzumab containing CD40 mAb ScFv | 127 |
| LC CD40 mAb containing Edrecolomab ScFv | 718 |
| LC Edrecolomab containing CD40 mAb ScFv | 140 |
| LC CD40 mAb containing Adecatumumab ScFv | 694 |
| LC Adecatumumab containing CD40 mAb ScFv | 153 |
| LC CD40 mAb containing Anetumab ScFv | 700 |
| LC Anetumab containing CD40 mAb ScFv | 166 |
| LC CD40 mAb containing huDS6 mAb ScFv | 724 |
| LC huDS6 mAb containing CD40 mAb ScFv | 179 |
| LC CD40 mAb containing Lifastuzumab ScFv | 736 |
| LC Lifastuzumab containing CD40 mAb ScFv | 192 |
| LC CD40 mAb containing Sacituzumab ScFv | 781 |
| LC Sacituzumab containing CD40 mAb ScFv | 205 |
| LC CD40 mAb containing PR1A3 mAb ScFv | 772 |
| LC PR1A3 mAb containing CD40 mAb ScFv | 218 |
| LC CD40 mAb containing Humanized PR1A3 mAb ScFv | 820 |
| LC Humanized PR1A3 mAb containing CD40 mAb ScFv | 817 |
| LC Humanized PR1A3 mAb containing CD40 mab scFv (LH) | 844 |
| LC CD40 mAb containing Humanized Ab2-3 mAb ScFv | 838 |
| LC Humanized Ab2-3 mAb containing CD40 mAb ScFv | 835 |
| LC Humanized Ab2-3 mAb containing CD40 mab scFv (LH) | 842 |
| LC CD40 mAb containing Claudiximab ScFv | 727 |
| LC Claudiximab containing CD40 mAb ScFv | 231 |
| LC CD40 mAb containing AMG595 mAb ScFv | 697 |
| LC AMG595 mAb containing CD40 mAb ScFv | 270 |
| LC CD40 mAb containing ABT806 mAb ScFv | 691 |
| LC ABT806 mAb containing CD40 mAb ScFv | 283 |
| LC CD40 mAb containing Sibrotuzumab ScFv | 787 |
| LC Sibrotuzumab containing CD40 mAb ScFv | 296 |
| LC CD40 mAb containing DS-8895a variant 1 mAb ScFv | 712 |
| LC DS-8895a variant 1 mAb containing CD40 mAb ScFv | 309 |
| LC CD40 mAb containing DS-8895a variant 2 mAb ScFv | 715 |
| LC DS-8895a variant 2 mAb containing CD40 mAb ScFv | 322 |
| LC CD40 mAb containing Anti-EphA2 mAb ScFv | 742 |
| LC Anti-EphA2 mAb containing CD40 mAb ScFv | 335 |
| LC CD40 mAb containing Narnatumab ScFv | 748 |
| LC Narnatumab containing CD40 mAb ScFv | 348 |
| LC CD40 mAb containing RG7841 mAb ScFv | 775 |
| LC RG7841 mAb containing CD40 mAb ScFv | 361 |
| LC CD40 mAb containing Farletuzumab ScFv | 721 |
| LC Farletuzumab containing CD40 mAb ScFv | 374 |
| LC CD40 mAb containing Mirvetuximab ScFv | 745 |
| LC Mirvetuximab containing CD40 mAb ScFv | 387 |
| LC CD40 mAb containing J591 variant 1 mAb ScFv | 730 |
| LC J591 variant 1 mAb containing CD40 mAb ScFv | 400 |
| LC CD40 mAb containing J591 variant 2 mAb ScFv | 733 |
| LC J591 variant 2 mAb containing CD40 mAb ScFv | 413 |
| LC CD40 mAb containing Rovalpituzumab ScFv | 778 |
| LC Rovalpituzumab containing CD40 mAb ScFv | 426 |
| LC CD40 mAb containing PF-06647020 mAb ScFv | 769 |
| LC PF-06647020 mAb containing CD40 mAb ScFv | 439 |
| LC CD40 mAb containing anti-PTK7 mAb ScFv | 688 |
| LC anti-PTK7 mAb containing CD40 mAb ScFv | 452 |
| LC CD40 mAb containing Ladiratuzumab ScFv | 784 |
| LC Ladiratuzumab containing CD40 mAb ScFv | 465 |
| LC CD40 mAb containing Cirmtuzumab ScFv | 706 |
| LC Cirmtuzumab containing CD40 mAb ScFv | 478 |
| LC CD40 mAb containing anti-MAGE-A3 mAb ScFv | 739 |
| LC anti-MAGE-A3 mAb containing CD40 mAb ScFv | 491 |
| LC CD40 mAb containing anti-NY-ESO-1 mAb ScFv | 754 |
| LC anti-NY-ESO-1 mAb containing CD40 mAb ScFv | 504 |
| LC CD40 mAb containing Trastuzumab ScFv | 793 |
| LC Trastuzumab containing CD40 mAb ScFv | 685 |
| LC CD40 mAb containing Trastuzumab scFv (LH, 25mer) | 798 |

TABLE 19
Exemplary Fusion Sequences - DEC-205 fusions via the light chain

| | SEQ |
|---|---|
| Fusion | ID NO: |
| LC DEC-205 mAb containing Pertuzumab ScFv | 767 |
| LC Pertuzumab containing DEC-205 mAb ScFv | 768 |
| LC DEC-205 mAb containing Cetuximab ScFv | 704 |
| LC Cetuximab containing DEC-205 mAb ScFv | 705 |
| LC DEC-205 mAb containing Panitumumab ScFv | 761 |
| LC Panitumumab containing DEC-205 mAb ScFv | 762 |
| LC DEC-205 mAb containing Nimotuzumab ScFv | 752 |
| LC Nimotuzumab containing DEC-205 mAb ScFv | 753 |
| LC DEC-205 mAb containing Zalutumumab ScFv | 803 |
| LC Zalutumumab containing DEC-205 mAb ScFv | 804 |
| LC DEC-205 mAb containing Onartuzumab ScFv | 758 |
| LC Onartuzumab containing DEC-205 mAb ScFv | 759 |
| LC DEC-205 mAb containing Patritumab ScFv | 764 |
| LC Patritumab containing DEC-205 mAb ScFv | 765 |
| LC DEC-205 mAb containing Clivatuzumab ScFv | 710 |
| LC Clivatuzumab containing DEC-205 mAb ScFv | 711 |
| LC DEC-205 mAb containing Sofituzumab ScFv | 791 |
| LC Sofituzumab containing DEC-205 mAb ScFv | 792 |
| LC DEC-205 mAb containing Edrecolomab ScFv | 719 |
| LC Edrecolomab containing DEC-205 mAb ScFv | 720 |
| LC DEC-205 mAb containing Adecatumumab ScFv | 695 |
| LC Adecatumumab containing DEC-205 mAb ScFv | 696 |
| LC DEC-205 mAb containing Anetumab ScFv | 701 |
| LC Anetumab containing DEC-205 mAb ScFv | 702 |
| LC DEC-205 mAb containing huDS6 mAb ScFv | 725 |
| LC huDS6 mAb containing DEC-205 mAb ScFv | 726 |
| LC DEC-205 mAb containing Lifastuzumab ScFv | 737 |
| LC Lifastuzumab containing DEC-205 mAb ScFv | 738 |
| LC DEC-205 mAb containing Sacituzumab ScFv | 782 |
| LC Sacituzumab containing DEC-205 mAb ScFv | 783 |
| LC DEC-205 mAb containing PR1A3 mAb ScFv | 773 |
| LC PR1A3 mAb containing DEC-205 mAb ScFv | 774 |
| LC DEC-205 mAb containing Humanized PR1A3 mAb ScFv | 821 |
| LC Humanized PR1A3 mAb containing DEC-205 mAb ScFv | 822 |
| LC DEC-205 mAb containing Humanized Ab2-3 mAb ScFv | 839 |
| LC Humanized Ab2-3 mAb containing DEC-205 mAb ScFv | 840 |
| LC DEC-205 mAb containing Claudiximab ScFv | 728 |
| LC Claudiximab containing DEC-205 mAb ScFv | 729 |
| LC DEC-205 mAb containing AMG595 mAb ScFv | 698 |
| LC AMG595 mAb containing DEC-205 mAb ScFv | 699 |
| LC DEC-205 mAb containing ABT806 mAb ScFv | 692 |
| LC ABT806 mAb containing DEC-205 mAb ScFv | 693 |
| LC DEC-205 mAb containing Sibrotuzumab ScFv | 788 |
| LC Sibrotuzumab containing DEC-205 mAb ScFv | 789 |
| LC DEC-205 mAb containing DS-8895a variant 1 mAb ScFv | 713 |
| LC DS-8895a variant 1 mAb containing DEC-205 mAb ScFv | 714 |
| LC DEC-205 mAb containing DS-8895a variant 2 mAb ScFv | 716 |
| LC DS-8895a variant 2 mAb containing DEC-205 mAb ScFv | 717 |
| LC DEC-205 mAb containing Anti-EphA2 mAb ScFv | 743 |
| LC Anti-EphA2 mAb containing DEC-205 mAb ScFv | 744 |
| LC DEC-205 mAb containing Narnatumab ScFv | 749 |
| LC Narnatumab containing DEC-205 mAb ScFv | 750 |
| LC DEC-205 mAb containing RG7841 mAb ScFv | 776 |
| LC RG7841 mAb containing DEC-205 mAb ScFv | 777 |
| LC DEC-205 mAb containing Farletuzumab ScFv | 722 |
| LC Farletuzumab containing DEC-205 mAb ScFv | 723 |
| LC DEC-205 mAb containing Mirvetuximab ScFv | 746 |
| LC Mirvetuximab containing DEC-205 mAb ScFv | 747 |
| LC DEC-205 mAb containing J591 variant 1 mAb ScFv | 731 |
| LC J591 variant 1 mAb containing DEC-205 mAb ScFv | 732 |
| LC DEC-205 mAb containing J591 variant 2 mAb ScFv | 734 |
| LC J591 variant 2 mAb containing DEC-205 mAb ScFv | 735 |
| LC DEC-205 mAb containing Rovalpituzumab ScFv | 779 |
| LC Rovalpituzumab containing DEC-205 mAb ScFv | 780 |
| LC DEC-205 mAb containing PF-06647020 mAb ScFv | 770 |
| LC PF-06647020 mAb containing DEC-205 mAb ScFv | 771 |
| LC DEC-205 mAb containing anti-PTK7 mAb ScFv | 689 |
| LC anti-PTK7 mAb containing DEC-205 mAb ScFv | 690 |
| LC DEC-205 mAb containing Ladiratuzumab ScFv | 785 |
| LC Ladiratuzumab containing DEC-205 mAb ScFv | 786 |
| LC DEC-205 mAb containing Cirmtuzumab ScFv | 707 |
| LC Cirmtuzumab containing DEC-205 mAb ScFv | 708 |
| LC DEC-205 mAb containing anti-MAGE-A3 mAb ScFv | 740 |
| LC anti-MAGE-A3 mAb containing DEC-205 mAb ScFv | 741 |
| LC DEC-205 mAb containing anti-NY-ESO-1 mAb ScFv | 755 |
| LC anti-NY-ESO-1 mAb containing DEC-205 mAb ScFv | 756 |
| LC DEC-205 mAb containing Trastuzumab ScFv | 794 |
| LC Trastuzumab containing DEC-205 mAb ScFv | 795 |
| LC DEC205 mAb containing Trastuzumab scFv (LH, 25mer) | 799 |

TABLE 20
Exemplary Fusion Sequences - CD40 fusion with DEC205

| | SEQ |
|---|---|
| Region | ID NO: |
| HC DEC205 variant 1 mAb with CD40 mAb ScFv | 243 |
| HC CD40 mAb with DEC205 variant 1 mAb ScFv | 242 |
| LC DEC205 variant 1 mAb containing CD40 mAb scFv | 244 |
| LC CD40 mAb containing DEC205 variant 1mAb scFv | 800 |
| HC DEC205 variant 2 mAb with CD40 mAb ScFv | 256 |
| HC CD40 mAb with DEC205 variant 2 mAb ScFv | 255 |
| LC DEC205 variant 2 mAb containing CD40 mAb scFv | 257 |
| LC CD40 mAb containing DEC205 variant 2 mAb scFv | 801 |

In some aspects, an antibody construct comprising: a) a first binding domain, wherein the first binding domain specifically binds to a tumor antigen; b) a second binding domain, wherein the second binding domain specifically binds to an antigen on an antigen presenting cell, wherein the antigen is a molecule on the antigen presenting cell; and c) an Fc domain; wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain.

In some aspects, an antibody construct for use in inducing immune cell activation comprising: a) a first binding domain, wherein the first binding domain specifically binds to a tumor antigen; b) a second binding domain, wherein the second binding domain specifically binds to an antigen on an antigen presenting cell, wherein the antigen is a molecule on the antigen presenting cell; and c) an Fc domain; wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the antibody construct upon binding to tumor antigen as measured by a cytokine release assay is greater than immune cell activation caused by the antibody construct in the absence of binding to tumor antigen.

In some embodiments, the second binding domain is attached to the Fc domain or the light chain of the first binding domain: a) as an Fc domain-second binding domain fusion peptide; b) as a light chain-second binding domain fusion peptide; or c) by a conjugation via a first linker. In some embodiments, the Fc domain is attached to the first binding domain: a) as an Fc domain-first binding domain fusion peptide; or b) by conjugation via a second linker.

In some embodiments, the Fc domain is attached to both the first binding domain and to the second binding domain as a first binding domain-Fc domain-second binding domain fusion peptide. In some embodiments, the first binding domain is attached to both the Fc domain and the second binding domain as a first binding domain-second binding domain-Fc domain fusion peptide. In some embodiments, the first binding domain and the Fc domain comprise an antibody and the second binding domain comprises a single chain variable fragment (scFv).

In some embodiments, the first binding domain has a set of variable region CDR sequences that comprises a set of variable region CDR sequences set forth in TABLE 3 or TABLE 4. In some embodiments, the second binding domain comprises a variable domain comprising a set of CDR sequences set forth in TABLE 11 or TABLE 12.

In some embodiments, the first binding domain comprises a variable region comprising VH and VL sequences at least 80% sequence identity to a pair of VH and VL sequences set forth in TABLE 5 or TABLE 6.

In some embodiments, the second binding domain comprises a variable region having VH and VL sequences having at least 80% sequence identity to a VH or VL sequence set forth in TABLE 13 or TABLE 14.

In some embodiments, the first binding domain comprises an amino acid sequence having at least 80% sequence identity to any sequence in TABLE 7 or TABLE 8. In some embodiments, the second binding domain comprises an amino acid sequence having at least 80% sequence identity to any sequence in TABLE 15 or TABLE 16.

In some embodiments, the second binding domain-Fc domain-first binding domain fusion peptide as described herein comprises an amino acid sequence having at least 80% sequence identity to a sequence in TABLE 9, TABLE 10, or TABLE 17. In some embodiments, the second binding domain-first binding domain-Fc domain fusion peptide as described herein comprises an amino acid sequence having at least 80% sequence identity to a sequence in TABLE 18 or TABLE 19.

In some embodiments, the first binding domain comprises an amino acid sequence having at least 80% sequence identity to any sequence in TABLE 7 or TABLE 15.

In some embodiments, the antibody construct or conjugate induces secretion of cytokines by an immune cell as measured by a cytokine release assay. In some embodiments, the cytokine is IFN-γ, IL-8, IL-12, IL-2, or a combination thereof. In some embodiments, the antibody construct or conjugate induces antigen presentation on a dendritic cell, B cell, macrophage, or a combination thereof.

### Recombinant Bispecific Antibodies

In certain embodiments, recombinant antibodies are provided that are “bispecific” that possess the ability to specifically bind to two different targets through at least two different antigen binding domains (referred to as recombinant bispecific antibodies, bispecific recombinant antibodies or the like). These antibodies have a target antigen binding domain and an effector antigen binding domain. The target antigen binding domain specifically binds to a tumor associated antigen. The effector antigen binding domain specifically binds to a molecule present on an antigen presenting cell (APC).

The recombinant bispecific antibodies can exhibit more potent immune activation when both antigen binding domains are bound to their respective antigens. One format for increasing immune activation when both antigen binding domains are bound to their respective antigens can be accomplished by a recombinant bispecific antibody coupled to an Fc comprising domain that exhibits reduced affinity to an Fc receptor. Another format for achieving an increased immune activation when both antigen binding domains are bound to their respective antigens can be accomplished by using a binding domain with a low avidity for its antigen as one of the antigen binding domains in the recombinant bispecific antibody. One binding domain of the bispecific antibody can specifically bind to a tumor associated antigen and another binding domain can specifically bind to a molecule on the surface of an antigen presenting cell (APC), such as a macrophage or dendritic cell. Thus, the two binding domains cooperate to bring APCs to cancerous cells or tumors allowing the APC to initiate/propagate a cancer cell/tumor specific immune response through cytokine release, chemokine release, or presentation of tumor associated antigens to effector or helper T cells. Cytokine release can be measured by a cytokine release assay. Chemokine release can be measured by an ELISA immunoassay. Presentation of tumor associated antigens can be measured by a cross-presentation assay.

Therapy with recombinant monoclonal or bispecific antibodies can generally be well tolerated; however, antibodies directed to immune response stimulating receptors on immune cells can result in systemic toxic release of cytokines and other immune modulators that can limit their clinical use or dose, thereby limiting their effectiveness in generating patient anti-tumor responses. This immune activation can be especially non-beneficial when it occurs systemically in the absence of tumor antigens. The systemic agonism exhibited by antibodies to many APC receptors can depend upon high affinity binding to the APC antigen and higher order cross-linking of the APC receptors by clustering of the cell bound antibodies. Many studies show this can be mediated by Fc gamma receptor (FcγR) binding to the Fc domain of the antibodies, and cross-linking of different antibody molecules and their bound APC immune stimulating receptors. An additional complication of cross-linking by FcγR can be antibody dependent cell mediated cytotoxicity (ADCC) of the APCs resulting in lowered immune response to tumors and pathogens. ADCC can be attributed to the antibody Fc region which binds to FcγRs on effector cells (e.g., NK cells). Two non-mutually exclusive solutions to the above can be contemplated. In one, elevating the threshold for FcγR binding can reduce excessive systemic immune activation and unwanted ADCC directed to APCs of antibody therapy. In the second, the affinity of the antibody for its APC target can be lowered so that effective agonistic binding of antibody molecules to APCs can be driven by avidity, preferentially found when the bispecific antibody is bound to its tumor antigen target. As described herein, the Fc comprising domain of the recombinant bispecific antibody can contain one or more mutations that can reduce binding to an FcγR. Alternatively, the Fc comprising domain can be derived from an IgG subclass that can bind to FcγRs with low affinity, for example IgG2. Fc receptors can be highly expressed on different antigen presenting cells such as dendritic cells, and their engagement can lead to activation of the immunostimulatory and antigen presenting function of these cells. By reducing binding of the Fc comprising domain to the FcγR, the threshold for APC activation can be raised. By raising the threshold for APC activation, the possibility of a damaging, or non-beneficial, immune/inflammatory response to healthy, non-cancerous tissue can be reduced. Attenuating activation by modifications made to the Fc comprising domain can result in superior bioavailability and lower side effects. Also described herein are recombinant bispecific antibodies with high affinity anti-tumor antigen binding and low affinity immune receptor binding such that APC activation can be increased when the recombinant bispecific antibody is bound to its tumor antigen. As a result, the antibodies of this disclosure generally can have a higher maximum tolerated dosage, and can be administered at levels higher than therapeutic antibodies not modified as described herein.

In some embodiments, a recombinant bispecific antibody comprises a target antigen binding domain, wherein the target antigen binding domain specifically binds to a tumor associated antigen; an effector antigen binding domain, wherein the effector antigen binding domain specifically binds to a molecule present on an antigen presenting cell and is not a lipocalin mutein; and an Fc comprising domain; wherein the recombinant bispecific antibody induces more potent immune activation when the recombinant bispecific antibody is bound to the tumor associated antigen and to the molecule on the antigen presenting cell as compared to when the recombinant bispecific antibody is bound to the molecule on the antigen presenting cell but not to the tumor associated antigen. In some aspects, immune activation by the recombinant bispecific antibody when bound to the tumor associated antigen is at least two times, five times, or ten times greater than immune activation by the recombinant bispecific antibody when the recombinant bispecific antibody is not bound to the tumor associated antigen.

In some embodiments, immune activation by the recombinant bispecific antibody is greater in the presence of cells having cell surface tumor associated antigen and antigen presenting cells having cell surface molecule as compared to immune activation in the absence of cells having cell surface tumor associated antigen. In some embodiments, immune activation by the recombinant bispecific antibody is greater in the presence of cells having cell surface tumor associated antigen and antigen presenting cells having cell surface molecule as compared to immune activation in the absence of cells having cell surface tumor associated antigen but in the presence of the antigen presenting cells.

In some embodiments, a recombinant bispecific antibody comprises a target antigen binding domain, wherein the target antigen binding domain specifically binds to a tumor associated antigen; an effector antigen binding domain, wherein the effector antigen binding domain specifically binds to a molecule present on an antigen presenting cell and is not a lipocalin mutein; and an Fc comprising domain; wherein when administered at the minimum anticipated biological effect level of the recombinant bispecific antibody, the biological effect of the recombinant bispecific antibody is increased when the recombinant bispecific antibody is bound to the tumor associated antigen as compared to the biological effect of the recombinant bispecific antibody when the recombinant bispecific antibody is not bound to the tumor associated antigen, but is bound to the molecule on the antigen presenting cell. In some embodiments, the biological effect is immune activation. In some aspects, biological effect of the recombinant bispecific antibody when the recombinant bispecific antibody is bound to the tumor associated antigen is at least two times, five times, or ten times greater than the biological effect of the recombinant bispecific antibody when the recombinant bispecific antibody is not bound to the tumor associated antigen, but is bound to the molecule on the antigen presenting cell.

In further aspects, an increase in biological effect (e.g., immune activation) is an increase one or more of: a secretion of one or more cytokines, a secretion of one or more chemokines, an expression level of one or more cell surface proteins associated with immune stimulation, and an activity of one or more immune cell functions. In still further aspects, the activity of one or more immune cell functions comprises antibody-dependent cell-mediated cytotoxicity, antibody dependent cellular phagocytosis, or antigen cross-presentation. In some aspects, the increase in immune activation is two times, three times, five times, or ten times greater than immune activation by the recombinant bispecific antibody when the recombinant bispecific antibody is not bound to the tumor associated antigen, but is bound to the molecule on the antigen presenting cell. In further aspects, the recombinant bispecific antibody induces tumor-cell directed antibody-dependent cell-mediated cytotoxicity.

In some aspects, a binding affinity of the effector antigen binding domain of the recombinant bispecific antibody to the molecule present on the antigen presenting cell is decreased compared to a binding affinity of the effector antigen binding domain of an antibody that lacks the target antigen binding domain, and wherein the binding affinity of the effector antigen binding domain of the recombinant bispecific antibody to the molecule present on the antigen presenting cell in a recombinant form is similar to the binding affinity of the effector antigen binding domain of an antibody that lacks the target antigen binding domain. In further aspects, a Kd of the binding affinity of the effector antigen binding domain of the recombinant bispecific antibody to the molecule present on the antigen presenting cell is increased by two times, five times, ten times, fifty times, or one-hundred times compared to the binding affinity of the effector antigen binding domain of an antibody that lacks the target antigen binding domain. In additional aspects, a Kd for binding of the effector antigen binding domain to the molecule present on the antigen presenting cell is less than 20 nM, less than 100 nM, or less than 500 nM.

In some aspects, the Fc comprising domain comprises one or more amino acid substitutions that result in an increase in affinity to one or more Fcγ receptors as compared to a wild-type Fc comprising domain. In other aspects, the Fc comprising domain comprises one or more amino acid substitutions that result in a decrease in affinity to one or more Fcγ receptors as compared to a wild-type Fc comprising domain. In some aspects, the Fc comprising domain comprises one or more amino acid substitutions that result in a loss of binding to one or more Fcγ receptors as compared to a wild-type Fc comprising domain.

These recombinant bispecific antibodies can be designed in many different configurations. In certain embodiments, the bispecific antibodies comprise two different antigen binding domains and an Fc comprising domain. In certain embodiments, the Fc comprising domain comprises an immunoglobulin constant region or portion thereof. The Fc comprising domain comprises at least one mutation that reduces the affinity of the Fc domain for an Fc receptor. In certain aspects, the Fc comprising domain comprises at least 2, 3, 4, 5, 6, 7, 8, 9, 10 or more mutations that reduce the affinity of the comprising domain for an Fc receptor. In certain embodiments, the Fc comprising domain comprises the Fc region of the IgG1 isotype subclass. In certain embodiments, the Fc comprising domain comprises the Fc region of the IgG2 isotype subclass. Alternatively, the Fc comprising domain comprises the Fc region of the IgG2A isotype subclass. The target antigen binding domain is arranged in any configuration that exhibits specific binding to a desired molecule and comprise at least one CDR sequence. For example, in certain embodiments, a binding domain comprises a Fab, an scFv, a heavy chain variable binding region capable of specific binding without a corresponding light chain (e.g., a camelid heavy chain antibody), or a nanobody. The effector antigen binding domain is arranged in any configuration that exhibits specific binding to a desired molecule and comprises at least one CDR sequence or polypeptide that specifically binds to a molecule present on the surface of an APC. Antigen binding domains can either be attached or coupled to the Fc comprising domain. For example, an antigen binding domain is attached to the Fc comprising domain when it is encoded by the same polypeptide (e.g., a fusion protein of two different polypeptides connected by peptide bonds between two amino acids). This attachment can be effected by using peptide synthesis techniques or production in a cell based system. The antigen binding domains can also be coupled to the Fc comprising domain via a covalent linkage that is not a peptide bond, for example, via a polyethylene glycol (PEG) linker.

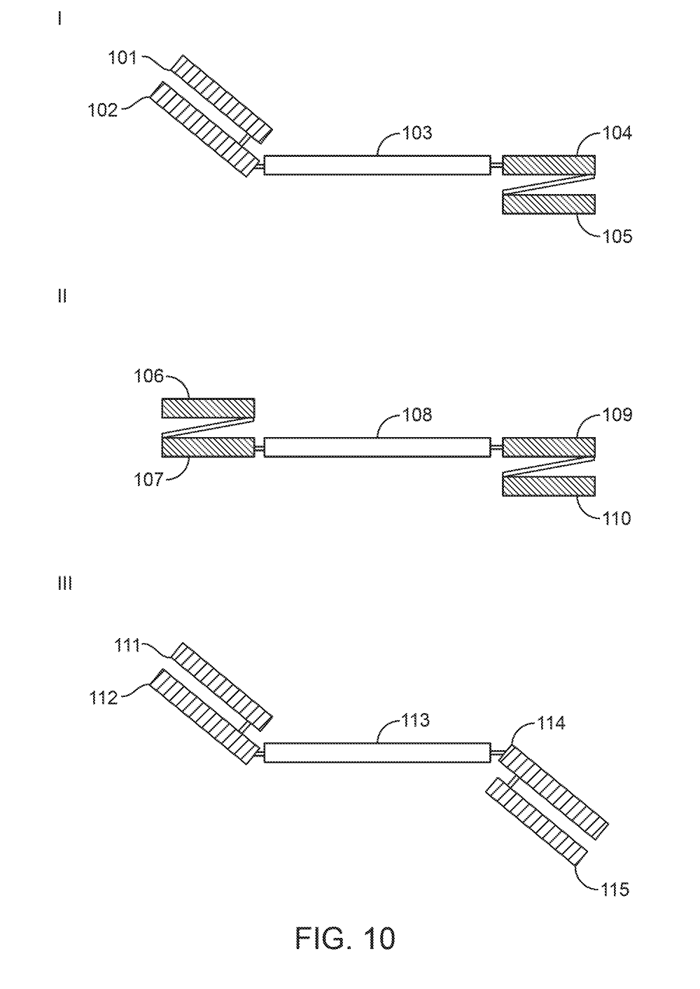

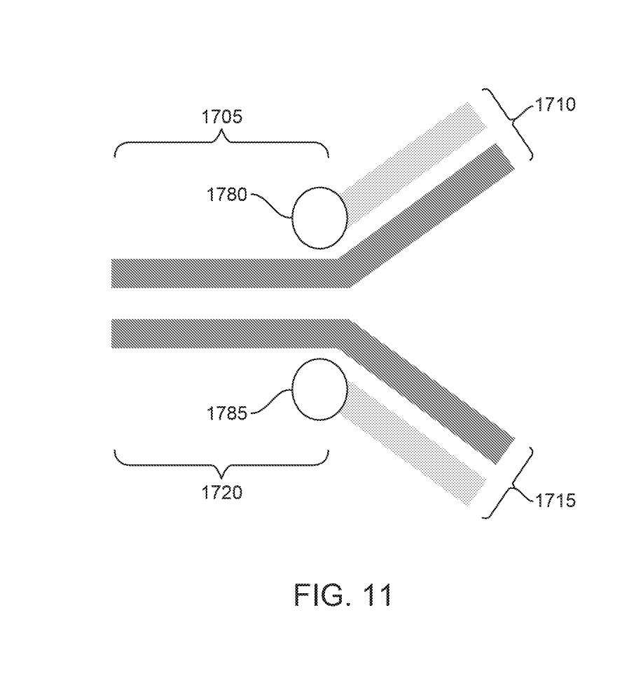

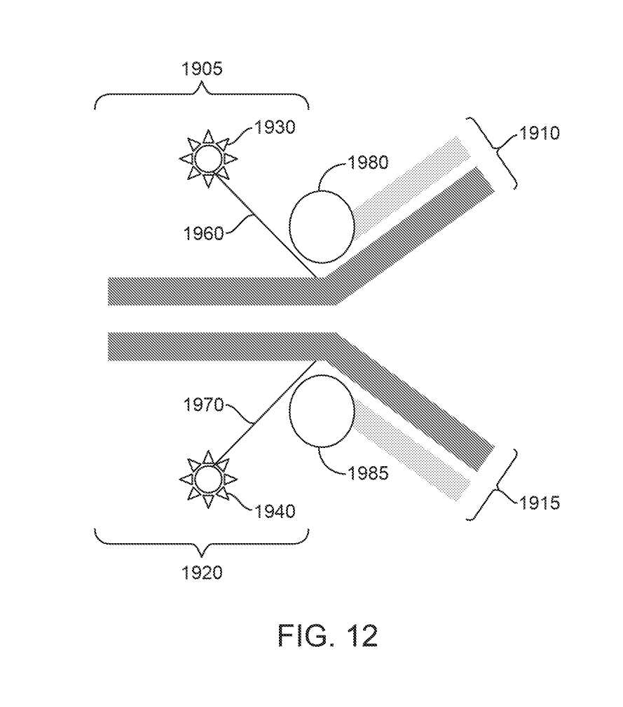

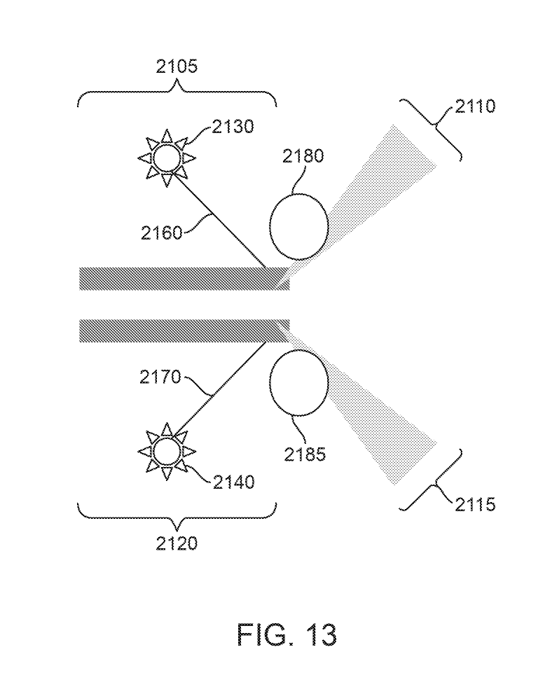

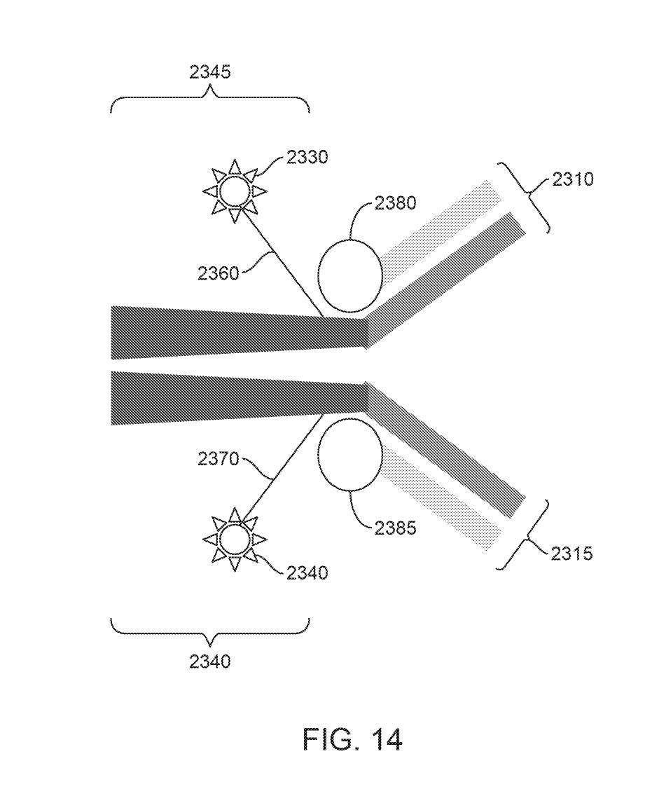

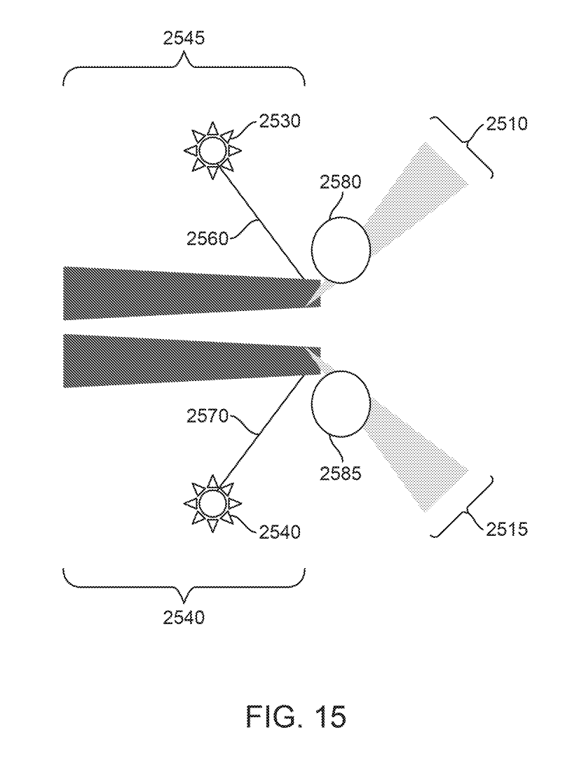

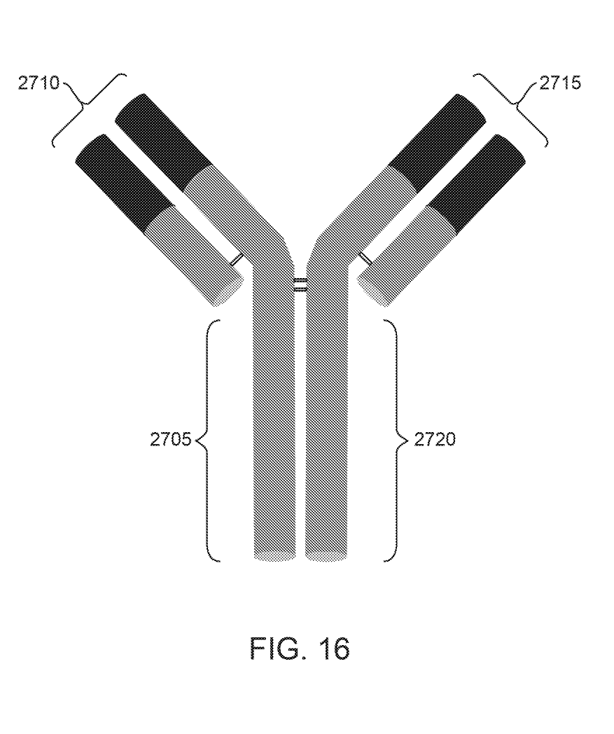

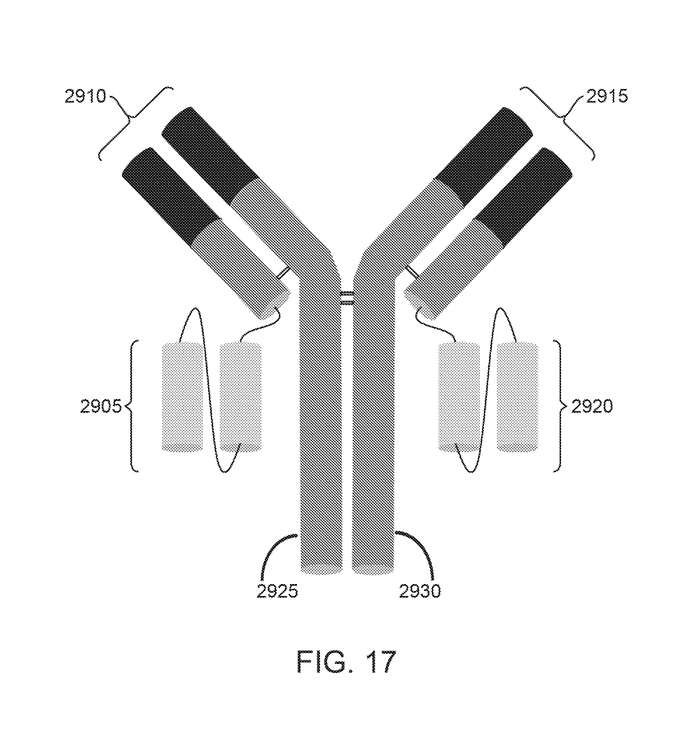

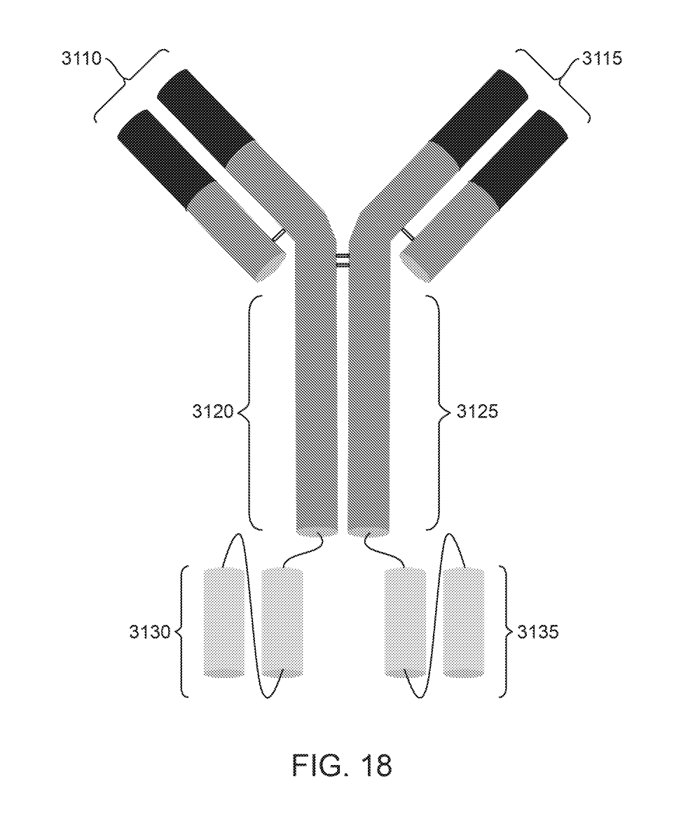

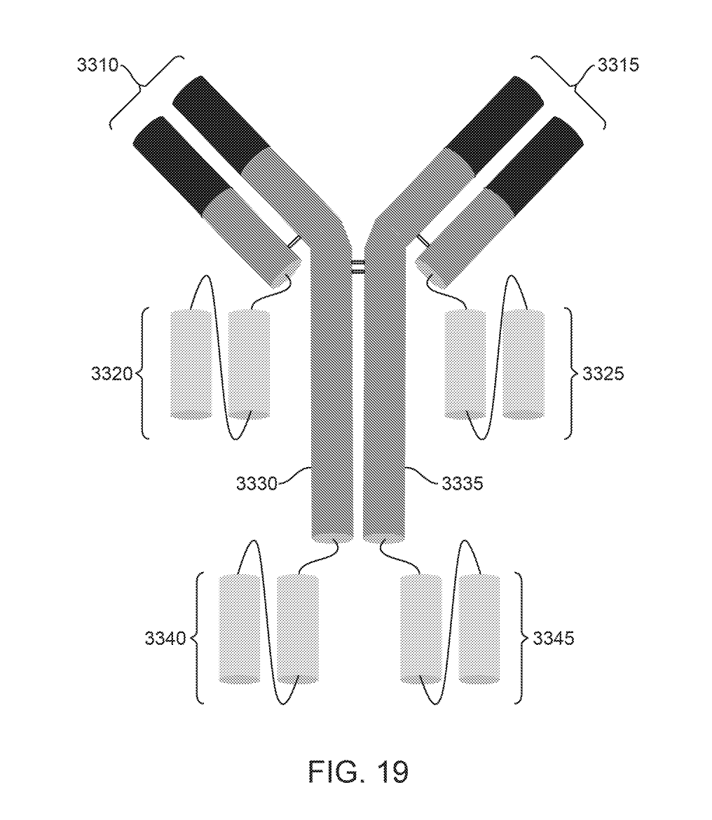

FIG. 10 depicts at least three examples of non-limiting embodiments of the recombinant bispecific antibodies. In embodiment I, a target antigen binding domain comprises a light chain variable and constant region 101 and a heavy chain variable and constant region 102. The heavy chain variable and constant region, either comprises an Fc region itself, or is attached to a polypeptide comprising an Fc region 103, which is in turn attached to an effector antigen binding domain comprising an scFv which comprises a heavy chain variable region 104 and a light chain variable region 105. In embodiment I the recombinant bispecific antibody is formed from two separate polypeptides. A single polypeptide encodes the heavy chain variable region 102, the Fc region 103, and the scFv 104 and 105, while a second polypeptide encodes the light chain variable region and constant region 101. The second polypeptide is covalently coupled to the first polypeptide by a disulfide linkage. In this embodiment, the target antigen binding domain approximates a Fab fragment. Each polypeptide comprises a leader sequence (not shown) that directs the polypeptide to the endoplasmic reticulum and the secretory pathway. The leader sequence is ultimately cleaved off of the polypeptides and the secreted molecule lacks the leader sequence. In a certain embodiment, the target antigen binding domain, the Fc region, and the effector antigen binding domain are attached via linkers that allow freedom of movement of the binding domains and prevent steric hindrance by the Fc region. The linkers can be any suitable length greater than 5, 6, 7, 8, 9, 10, 11, 12, 13, 14, or 15 amino acids. In certain embodiments, the linkers are resistant to proteases. In embodiment I, while the target antigen binding domain is depicted at the N-terminus and the effector antigen binding domain scFv at the C-terminus, the orientation can be reversed with the effector antigen binding domain at the N-terminus and the target antigen binding domain at the C-terminus. The target antigen binding domain specifically binds to a tumor associated antigen; the effector antigen binding domain specifically binds to a molecule present on an antigen presenting cell. It is also envisioned that the Fab fragment can be expressed separately from the Fc region-scFv 103-105 and coupled by chemical means through the use of a linker.

In alternative embodiment II, the recombinant bispecific antibody comprises a single polypeptide with two different antigen binding domains that comprise scFvs. A first scFv comprises a light chain variable region 106 attached to a heavy chain variable region 107, which is in turn attached to a polypeptide comprising an Fc region 108, which is attached to a second scFv comprising a heavy chain variable region 109 and a light chain variable region 110. In certain embodiments, the target antigen binding domain specifically binds to a tumor associated antigen, while the effector antigen binding domain specifically binds to a molecule present on an antigen presenting cell.

In alternative embodiment III, the recombinant bispecific antibody comprises two different Fab fragments (111 and 112; 114 and 115) that are coupled or attached to a polypeptide comprising an Fc region 113.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; c) an Fc comprising domain; and d) an immune-stimulatory compound attached to the recombinant bispecific antibody by a linker; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

In some aspects, a recombinant bispecific antibody, comprising: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; and c) an Fc comprising domain; and d) an immune-stimulatory compound attached to the recombinant bispecific antibody by a linker; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; c) an Fc comprising domain; and d) an immune-stimulatory compound attached to the recombinant bispecific antibody by a linker; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

In some aspects, a recombinant bispecific antibody, comprises: a) a target antigen binding domain, wherein the target antigen binding domain specifically binds to a tumor associated antigen; b) an effector antigen binding domain, wherein the effector antigen binding domain specifically binds to an antigen on an antigen presenting cell and is an antibody antigen binding domain, wherein the antigen is a molecule on the antigen presenting cell; and c) a domain comprising an Fc region; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

In some aspects, a recombinant bispecific antibody, comprising: a) a target antigen binding domain, wherein the target antigen binding domain specifically binds to a tumor associated antigen; b) an effector antigen binding domain, wherein the effector antigen binding domain specifically binds to an antigen on an antigen presenting cell and wherein the antigen is a molecule on the antigen presenting cell; and c) an Fc comprising domain; and d) an immune-stimulatory compound attached to the recombinant bispecific antibody by a linker; wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

In some embodiments, the Fc comprising domain is linked C-terminal to the target antigen binding domain and N-terminal to the effector antigen binding domain. In some embodiments, the linker links the immune-stimulatory compound to the Fc comprising domain.

In some embodiments, the immune cell activation is measured by a cytokine release assay.

In some embodiments, the immune cell activation by the recombinant bispecific antibody when the recombinant bispecific antibody is bound to the tumor associated antigen and to the antigen on the antigen presenting cell is at least two times, five times, or ten times greater than immune activation by the recombinant bispecific antibody when the recombinant bispecific antibody is bound to the antigen on the antigen presenting cell but not to the tumor associated antigen as measured by the cytokine release assay.

In some embodiments, the immune cell activation by the recombinant bispecific antibody in the presence of cells having cell surface tumor associated antigen and antigen presenting cells having cell surface antigen is at least two times, five times, or ten times greater than immune cell activation by the recombinant bispecific antibody in the absence of the cells having cell surface tumor associated antigen as measured by the cytokine release assay.

In some embodiments, the immune cell activation comprises an increase in one or more of: a) a secretion of one or more cytokines as measured by the cytokine release assay, b) a secretion of one or more chemokines as measured by an ELISA immunoassay, c) an expression level of one or more cell surface proteins associated with immune stimulation as measured by FACS, and d) an activity of one or more immune cell functions.

In some embodiments, the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay.

In some embodiments, the recombinant bispecific antibody induces tumor-cell directed antibody-dependent cell-mediated cytotoxicity.

In some aspects, method of making a recombinant bispecific antibody comprises: a) producing an antibody construct comprising: i) a target antigen binding domain, wherein the target antigen binding domain specifically binds to a tumor associated antigen; ii) an effector antigen binding domain, wherein the effector antigen binding domain specifically binds to an antigen on an antigen presenting cell and the antigen is a molecule on the antigen presenting cell, wherein the antigen is a molecule on the antigen presenting cell; iii) an Fc comprising domain; and b) linking an immune-stimulatory compound to the antibody construct, wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

### Constant Regions and Fe Regions

The recombinant bispecific antibodies described herein comprise a constant region, or portion thereof, in addition to a variable region (or CDR sequences derived from a variable region). The heavy chain constant region (CH) comprises three or four domains abbreviated CH1, CH2, CH3, and CH4, depending on the isotype of the constant region. The domains are located at the C-terminal end of the full heavy chain polypeptide, C-terminal to the variable region. The light chain constant region (CL) is much smaller that the CH and is located at the C-terminal end of the full light chain polypeptide, C-terminal to the variable region. In certain embodiments, the recombinant bispecific antibodies herein comprise a constant region lacking a CH4 region. The constant region is highly conserved and comprises different isotypes, that are associated with slightly different functions and properties. In certain embodiments, the constant region is not required for antibody binding to a target antigen. In certain embodiments, the constant regions of the recombinant bispecific antibodies, both heavy and light chains are not required for antibody binding to a target antigen. In certain embodiments, the recombinant bispecific antibodies lack one or more of a light chain constant region, heavy chain constant region, or both. Most monoclonal antibodies are of an IgG isotype; which is further divided into four subclasses IgG1, IgG2, IgG3, and IgG4. In certain embodiments, the recombinant bispecific antibodies comprise any IgG subclass. In certain embodiments, the IgG subclass comprises IgG. In certain embodiments, the IgG subclass comprises IgG2. In certain embodiments, the IgG subclass comprises IgG3. In certain embodiments, the IgG subclass comprises IgG4.

The recombinant bispecific antibodies described herein comprise an Fc comprising domain in addition to target and effector antigen binding domains. Natural antibodies comprise a fragment crystallizable region (Fc region) that is responsible for binding to complement and Fc receptors. The Fc region comprises the CH2, CH3, and CH4 regions of the antibody molecule, and is responsible for activating complement and antibody dependent cell cytotoxicity (ADCC). Much of the variability in Fc function can be attributed to the CH2 region. The Fc region also contributes to an antibody's serum half-life. IgG isotype subclass Fc regions exhibit varying affinity for Fc receptors. IgG1 and IgG3 exhibit high affinity binding to all Fc receptors, both with respect to affinity and amount of different Fc receptors. IgG2, however, exhibits the lowest binding affinity toward various Fc receptors, with IgG4 showing an intermediate binding affinity. In certain embodiments, the Fc region is attached to one or both of the antigen binding domains. In certain embodiments, the recombinant bispecific antibodies comprise an Fc region derived from the IgG2 isotype subclass.

There are several different Fc receptors with varying affinity for the antibody Fc region, ability to activate signaling though the Fc receptor, and expression on immune cells. For example, FcγRI possess a very high affinity for IgG (KD of approximately 1×10-8 M).

The Fc comprising domain of the recombinant bispecific antibodies comprises one or more, two or more, three or more, or four or more amino acid substitutions that decrease binding of the antibody to an Fc receptor. In certain embodiments, the Fc receptor comprises FcγRI (CD64), FcγRIIA (CD32), FcγRIIIA (CD16a), FcγRIIIB (CD16b), or any combination thereof. In certain embodiments, the Fc comprising domain of the recombinant bispecific antibodies comprise one or more amino acid substitutions that increase the serum half-life of the antibody. In certain embodiments, the one or more amino acid substitutions that increase the serum half-life of the antibody increase affinity of the antibody to the neonatal Fc receptor (FcRn). In order to decrease binding affinity of an Fc region to Fc receptor the Fc comprising domain may comprise one or more mutations that has the effect of reducing the affinity of the Fc region to an Fc receptor. In certain embodiments, the one or more mutations comprise any one or more of IgG1 heavy chain mutations corresponding to E233P, L234V, L234A, L235A, L235E, AG236, G237A, E318A, K320A, K322A, A327G, A330S, or P331S according to the EU index of Kabat numbering.

The Fc comprising domain of the recombinant bispecific antibodies comprises one, two, three, four or more amino acid substitutions that decrease binding of the antibody to an Fc receptor. The one, two, three, four or more amino acid substitutions decrease binding by at least two fold, three fold, four fold, five fold or ten fold. In certain embodiments, the one, two, three, four or more amino acid substitutions completely abolish binding to an Fc receptor (Fc null). In certain embodiments, the one, two, three, four or more amino acid substitutions are differences compared to any one of SEQ ID NO: 1314, SEQ ID NO: 1315, SEQ ID NO: 1316, or SEQ ID NO: 1317. In certain embodiments, the one, two, three, four or more amino acid substitutions are differences compared to any one of SEQ ID NO: 1314, SEQ ID NO: 1315, SEQ ID NO: 1316, or SEQ ID NO: 1317. In certain embodiments, one, two, three, four or more amino acid substitutions are differences compared to any one of SEQ ID NO: 1319, SEQ ID NO: 1320, SEQ ID NO: 1321, or SEQ ID NO: 1322. In certain embodiments, one, two, three, four or more amino acid substitutions are differences compared to any one of SEQ ID NO: 1319, SEQ ID NO: 1320, SEQ ID NO: 1321, or SEQ ID NO: 1322. In certain embodiments, the Fc comprising domain comprises an amino sequence at least 80%, 90%, 95%, 98%, 99%, or 100% identical to the amino acid sequence set forth in SEQ ID NO: 1313. In certain embodiments, the Fc comprising domain comprises an amino sequence at least 80%, 90%, 95%, 98%, 99%, or 100% identical to the amino acid sequence set forth in SEQ ID NO: 1318.

Alternatively, an amino acid change in an Fc comprising domain of the recombinant bispecific antibodies can allow the recombinant bispecific antibody to bind to at least one Fc receptor with greater affinity compared to a wild-type Fc region. An Fc comprising domain can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc comprising domain can be an Fc region of an anti-CD40 antibody.

The Fc comprising domain of the recombinant bispecific antibody can comprise a sequence of the IgG1 isoform that has been modified from the wild-type IgG sequence. A modification can comprise a substitution at more than one amino acid residue such as at 5 different amino acid residues including L235V/F243L/R292P/Y300L/P396L (IgG1VLPLL). The numbering of amino acids residues described herein is according to the EU index as in Kabat. The 5 amino acid residues can be located in a portion of a recombinant bispecific antibody sequence which can encode an Fc comprising domain of the recombinant bispecific antibody and in particular, can be located in portions of the Fc region that can bind to Fc receptors (i.e., the Fc domain). A modification can comprise a substitution at more than one amino acid residue such as at 2 different amino acid residues including S239D/I332E (IgG1DE) according to the EU index of Kabat numbering. The 2 amino acid residues can be located in a portion of a recombinant bispecific antibody sequence which encodes an Fc comprising domain of the recombinant bispecific antibody and in particular, are located in portions of the Fc region that can bind to Fc receptors (i.e., the Fc domain). A modification can comprise a substitution at more than one amino acid residue such as at 3 different amino acid residues including S298A/E333A/K334A (IgG1AAA) according to the EU index of Kabat numbering. The 3 amino acid residues can be located in a portion of a recombinant bispecific antibody sequence which can encode an Fc comprising domain of the recombinant bispecific antibody and in particular, can be located in portions of the Fc region that can bind Fc receptors (i.e., the Fc domain).

The Fc comprising domain can be from a monoclonal anti-CD40 human antibody comprising a sequence of the IgG isoform that has been modified from the wild-type IgG sequence. A modification can comprise a substitution at more than one amino acid residue such as at 5 different amino acid residues including L235V/F243L/R292P/Y300L/P396L (SBT-040-G1VLPLL). The numbering of amino acids residues described herein is according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (SBT-040-G1DE) according to the EU index of Kabat numbering. The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (SBT-040-G1AAA) according to the EU index of Kabat numbering. The 3 amino acid residues can be located in a portion of an antibody sequence which can encode an Fc region of the antibody and in particular, can be located in portions of the Fc region that can bind to Fc receptors (i.e., the Fc domain).

Binding of Fc receptors to an Fc comprising domain can be affected by amino acid substitutions. For example, a recombinant bispecific antibody can comprise an amino acid sequence of a heavy chain of human anti-CD40 monoclonal antibody with modifications to a wild-type IgG1 Fc domain (L235V/F243L/R292P/Y300L/P396L). Binding of some Fc receptors to this Fc comprising domain with the L235V/F243L/R292P/Y300L/P396L amino acid modifications can be enhanced compared to wild-type as result of the L235V/F243L/R292P/Y300L/P396L amino acid modifications. The recombinant bispecific antibody can comprise an amino acid sequence of a heavy chain of human anti-CD40 monoclonal antibody with modifications to a wild-type IgG1 Fc domain (S239D/I332E). Binding of Fc receptors to the Fc comprising domain of the recombinant bispecific antibody can be enhanced compared to wild-type as a result of the S239D/I332E amino acid modification. The recombinant bispecific antibody can comprise an amino acid sequence of a heavy chain of a human anti-CD40 monoclonal antibody with modifications to a wild-type IgG1 Fc domain (S298A/E333A/K334A). Binding of Fc receptors to a Fc comprising domain of a recombinant bispecific antibody can be enhanced compared to wild-type as a result of the S298A/E333A/K334A amino acid modification.

In some embodiments, the Fc comprising domain has one or more amino acid substitutions that decrease the binding affinity to one or more Fcγ receptors as compared to a wild-type Fc comprising domain.

In some embodiments, the Fc comprising domain is linked to the target antigen binding domain and to the effector antigen binding domain.

In some embodiments, the Fc comprising domain comprises one or more amino acid substitutions that reduce the affinity of the Fc comprising domain to an Fc receptor compared to the affinity of a reference Fc comprising domain to the Fc receptor in the absence of the one or more amino acid substitutions.

In some embodiments, reference Fc comprising domain is selected from the group consisting of an Fc comprising domain having the amino acid sequence of SEQ ID NO: 1314, SEQ ID NO: 1315, SEQ ID NO: 1316, and SEQ ID NO: 1317.

In some embodiments, reference Fc comprising domain comprises an amino acid sequence selected from the group consisting of SEQ ID NO: 1319, SEQ ID NO: 1320, SEQ ID NO: 1321, and SEQ ID NO: 1322.

In some embodiments, the Fc comprising domain comprises a human IgG₁ Fc Region. In some embodiments, the one or more amino acid substitutions comprise L234A, L235A, G237A, and K322A, according to the EU index of Kabat. In some embodiments, the one or more amino acid substitutions comprise E233P, L234V, L235A, AG236, A327G, A330S, and P331S, according to the EU index of Kabat. In some embodiments, the Fc comprising domain comprises a human IgG₂ Fc Region.

In some embodiments, the one or more amino acid substitutions comprises K322A, according to the EU index of Kabat.

In some embodiments, the Fc comprising domain comprises a human IgG₂ₐ Fc Region In some embodiments, the one or more amino acid substitutions comprises L235E, E318A, K320A, K322A, according to the EU index of Kabat.

In some embodiments, the Fc comprising domain is an Fc null. In some embodiments, the Fc comprising domain has the amino acid sequence of SEQ ID NO: 1313.

In some embodiments, the Fc comprising domain comprises the amino acid sequence of SEQ ID NO: 1318.

In some embodiments, the Fc comprising domain is linked C-terminal to the target antigen binding domain and has the amino acid sequence of SEQ ID NO: 1311.

### Antigen Binding Domains

In certain embodiments, the recombinant bispecific antibodies comprise a target and effector antigen binding domain. Each antigen binding domain comprises one or more complementarity determining regions (CDRs). A CDR is a part of an immunoglobulin (antibody) variable region that is primarily responsible for the antigen binding specificity of the antibody. CDR regions are highly variable from one antibody to the next, even when the antibody specifically binds the same target or epitope. A heavy chain variable region comprises three CDR regions, abbreviated HCDR1, HCDR2, and HCDR3; and a light chain variable region comprises three CDR regions, abbreviated LCDR1, LCDR2, and LCDR3. These CDR regions are ordered consecutively in the variable region with the CDR1 being the most N-terminal and the CDR3 being the most C-terminal. Interspersed between the CDRs are framework regions which contribute to the structure and display much less variability than the CDR regions. A heavy chain variable region comprises four framework regions, abbreviated HFR1, HFR2, HFR3, and HFR4; and a light chain variable region comprises four framework regions, abbreviated LFR1, LFR2, LFR3, and LFR4. Complete full sized bivalent antibodies comprising two heavy and light chains will comprise: 12 CDRs, with three unique heavy chain CDRs and three unique light chain CDRs; 16 FR regions, with four unique heavy chain FR regions and four unique light chain FR regions. In certain embodiments, the antibodies described herein minimally comprise three heavy chain CDRs. In certain embodiments, the antibodies described herein minimally comprise three light chain CDRs. In certain embodiments, the antibodies described herein minimally comprise three heavy chain CDRs and three light chain CDRs. The CDRs may be expressed as an scFv or a traditional heavy chain and light chain pair. CDRs are identified from sequences using different numbering systems such as the Kabat or the IMGT numbering systems. In certain embodiments, the antibodies described herein comprise variable regions of non-human origin. In certain embodiments, the antibodies described herein comprise CDRs of non-human origin. In certain embodiments, the antibodies described herein comprise variable regions of mouse origin. In certain embodiments, the antibodies described herein comprise CDRs of mouse origin.

In some embodiments, the tumor associated antigen is an antigen selected from the group consisting of CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PD-L1, CD204, CD206, CD301, VTCN1, and VISTA. In some embodiments, the tumor associated antigen is Her2/neu or p185HER2.

### Target Antigen Binding Domain

The recombinant bispecific antibodies comprise a target antigen binding domain that specifically binds to a tumor associated antigen. As described herein, a “tumor associated antigen” refers to a molecular marker that is can be expressed by a neoplastic tumor cell and/or within a tumor microenvironment. For example, a tumor associated antigen can be an antigen expressed on a cell associated with a tumor, such as a neoplastic cell, stromal cell, endothelial cell, fibroblast, or tumor-infiltrating immune cell. For example, the tumor associated antigen Her2/Neu is overexpressed by certain types of breast and ovarian cancer. A tumor antigen may also be ectopically expressed by a tumor and contribute to deregulation of the cell cycle, reduced apoptosis, metastasis, or escape from immune surveillance. Tumor associated antigens are generally proteins or polypeptides derived therefrom, but can be glycans, lipids, or other small organic molecules. Additionally, a tumor antigen can arise through increases or decreases in post-translational processing exhibited by a cancer cell compared to a normal cell, for example, protein glycosylation, protein lipidation, protein phosphorylation, or protein acetylation. In certain embodiments, the target antigen binding domain specifically binds to a tumor associated antigen selected from the group consisting of CD5, CD19, CD20, CD25, CD37, CD30, CD33, CD45, CAMPATH-1, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Ley, CA-125, CA19-9, epidermal growth factor, p185HER2, IL-2 receptor, fibroblast activation protein, tenascin, a metalloproteinase, endosialin, vascular endothelial growth factor, avB3, WT1, LMP2, HPV E6, HPV E7, EGFRvIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7-H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, FAP, RON, LY6E, FRA, DLL3, PTK7, LIV1, ROR1, Fos-related antigen 1, VEGFR, endoglin, PD-L, CD204, CD206, CD301, VTCN1, or VISTA.

In certain embodiments, the target antigen binding domain specifically binds to a tumor associated antigen selected from the group consisting of GD2, GD3, GM2, Ley, polysialic acid, fucosyl GM1, GM3, Tn, STn, sLe(animal), or GloboH or having an amino acid sequence comprising at least 80%, 90%, 95%, 97%, 98%, 99% or 100% homology to CD5, CD19, CD20, CD25, CD37, CD30, CD33, CD45, CAMPATH-1, HLD-DR, carcinoembryonic antigen (CEA), TAG-72, EpCAM, MUC1, folate-binding protein, A33, G250, prostate-specific membrane antigen (PSMA), ferritin, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, CYP1B1, PLAV1, BORIS, ETV6-AML, NY-BR-1, RGS5, SART3, Carbonic anhydrase IX, PAX5, OY-TES1, Sperm protein 17, LCK, HMWMAA, AKAP-4, SSX2, XAGE 1, B7-H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PD-L1, CD204, CD206, CD301, VTCN1, or VISTA. In certain embodiments, the target antigen binding domain specifically binds to a tumor associated antigen comprising Her2/Neu (CD340). In certain embodiments, the target antigen binding domain specifically binds to a tumor associated antigen comprising at least 80%, 90%, 95%, 97%, 98%, 99% or 100% homology to CD5, CD19, CD20, CD25, CD37, CD30, CD33, CD45, CAMPATH-1, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PSMA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B, polysialic acid, MYCN, RhoC, TRP-2, fucosyl GM1, mesothelin, PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, MSLN, CA6, NAPI2B, TROP2, CLDN18.2, FAP, RON, LY6E, FRA, DLL3, PTK7, LIV1, ROR1, Fos-related antigen 1, VEGFR, endoglin, PDL-1, CD204, CD206, CD301, VTCN1, or VISTA.

The antigen binding domain that specifically binds a tumor associated antigen can be derived from any known antibody with the ability to bind to an antigen shown to be expressed by cancer cells. In certain embodiments, the target antigen binding domain is derived from an antibody selected from the group of Etaracizumab (Abegrin), Tacatuzumab tetraxetan, Bevacizumab (Avastin), Labetuzumab, Cetuximab (Erbitux), Obinutuzumab (Gazyva), Trastuzumab (Herceptin), Clivatuzumab, Rituximab (MabThera, Rituxan), Gemtuzumab of Gemtuzumab ozogamicin (Mylotarg), Girentuximab (Rencarex), or Nimotuzumab (Theracim, Theraloc). In certain embodiments, the target antigen binding domain is derived from Pertuzumab (Perjeta). A target antigen binding domain is derived from a known antibody when it comprises one or more CDR sequences identical to one or more CDR sequences of the known antibody. In certain embodiments, a target antigen binding domain is derived from a known antibody when it comprises three HCDR sequences identical to three HCDR sequences of the known antibody. In certain embodiments, a target antigen binding domain is derived from a known antibody when it comprises three LCDR sequences identical to three LCDR sequences of the known antibody.

In certain embodiments, the target antigen binding domain of the recombinant bispecific antibody is derived from pertuzumab. Pertuzumab is a monoclonal antibody that specifically binds the Her2/Neu antigen. The heavy chain amino acid sequence for pertuzumab is set forth in SEQ ID NO: 11; the light chain amino acid sequence for pertuzumab is set forth in SEQ ID NO: 16. In certain embodiments, the target antigen binding domain comprises a CDR sequence identified from either of SEQ ID NO: 11 or SEQ ID NO: 16 identified using the Kabat, IMGT or Chothia method. In certain embodiments, the target antigen binding domain comprises an amino acid sequence with at least 80%, 90%, 95%, 98%, 99%, or 100% identity to SEQ ID NO: 11. In certain embodiments, the target antigen binding domain comprises an amino acid sequence with at least 80%, 90%, 95%, 98%, 99%, or 100% identity to SEQ ID NO: 16. In certain embodiments, the target antigen binding domain comprises an amino acid sequence with at least 80%, 90%, 95%, 98%, 99%, or 100% identity to SEQ ID NO: 11 and SEQ ID NO: 16.

The heavy chain variable region amino acid sequence for pertuzumab is set forth in SEQ ID NO: 12; the light chain variable region amino acid sequence for pertuzumab is set forth in SEQ ID NO: 17. In certain embodiments, the target antigen binding domain comprises a CDR sequence identified from either of SEQ ID NO: 12 or SEQ ID NO: 17 identified using the Kabat, IMGT or Chothia method. In certain embodiments, the target antigen binding domain comprises an amino acid sequence with at least 80%, 90%, 95%, 98%, 99%, or 100% identity to SEQ ID NO: 12. In certain embodiments, the target antigen binding domain comprises an amino acid sequence with at least 80%, 90%, 95%, 98%, 99%, or 100% identity to SEQ ID NO: 17. In certain embodiments, the target antigen binding domain comprises an amino acid sequence with at least 80%, 90%, 95%, 98%, 99%, or 100% identity to SEQ ID NO: 12 and SEQ ID NO: 17.

In certain embodiments, the target antigen binding domain may comprise the CDR sequence of pertuzumab. The amino acid sequence of the HCDR1 of pertuzumab is set forth in SEQ ID NO: 13; the amino acid sequence of the HCDR2 of pertuzumab is set forth in SEQ ID NO: 14; the amino acid sequence of the HCDR3 of pertuzumab is set forth in SEQ ID NO: 15; the amino acid sequence of the LCDR1 of pertuzumab is set forth in SEQ ID NO: 18; the amino acid sequence of the LCDR2 of pertuzumab is set forth in SEQ ID NO: 19; and the amino acid sequence of the LCDR3 of pertuzumab is set forth in SEQ ID NO: 20. In certain embodiments, the target antigen binding domain comprises any one of SEQ ID NO: 13-SEQ ID NO: 15 and SEQ ID NO: 18-SEQ ID NO: 20. In certain embodiments, the target antigen binding domain comprises all of SEQ ID NO: 13-SEQ ID NO: 15 and SEQ ID NO: 18-SEQ ID NO: 20. In certain embodiments, the target antigen binding domain comprises any one of SEQ ID NO: 13-SEQ ID NO: 15. In certain embodiments, the target antigen binding domain comprises any one of SEQ ID NO: 18-SEQ ID NO: 20. In certain embodiments, the target antigen binding domain comprises a sequence at least all of SEQ ID NO: 13 and SEQ ID NO: 15. In certain embodiments, the target antigen binding domain comprises all of SEQ ID NO: 18 and SEQ ID NO 20. The CDR sequences above may be incorporated into the target antigen binding domain by any method of recombinant DNA technology. The incorporated CDRs can have any amount of amino acid sequence identity to any one of SEQ ID NO: 13-SEQ ID NO: 15 and SEQ ID NO: 18-SEQ ID NO: 20 that still retains the specific binding of pertuzumab, for example, 80%, 90%, 95%, 98%, 99%, or 100% identity.

In certain embodiments, the recombinant bispecific antibody comprises a target antigen binding domain and an Fc region that are attached in a single polypeptide, resulting in a fusion. In certain embodiments, the recombinant bispecific antibody comprises a target antigen binding domain and an Fc comprising domain as a single polypeptide, which is a fusion. In certain embodiments, the target antigen binding domain attached to the Fc region comprises an amino acid sequence set forth in SEQ ID NO: 1311. In certain embodiments, the target antigen binding domain attached to the Fc region comprises an amino acid sequence at least 80%, 90%, 95%, 98%, 99%, or 100% identical to that set forth in SEQ ID NO: 1311.

In certain embodiments, the recombinant bispecific antibody comprises a target antigen binding with at least 80%, 90%, 95%, 98%, 99%, or 100% sequence identity to 6, 7, 8, 9, 10, 11, 12 or more contiguous amino acids between amino acid 20 and amino acid 110 of SEQ ID NO: 12 and at least 80%, 90%, 95%, 98%, 99%, or 100% sequence identity to 6, 7, 8, 9, 10, 11, 12 or more contiguous amino acids between amino acid 20 and amino acid 105 of SEQ ID NO: 17.

In some embodiments, the target antigen binding domain comprises an immunoglobulin heavy chain variable region or antigen binding fragment thereof and an immunoglobulin light chain variable region or antigen binding fragment thereof.

In some embodiments, the target antigen binding domain comprises a single chain variable region fragment (scFv).

In some embodiments, the target antigen binding domain comprises the following CDRs: a) HCDR1 comprising an amino acid sequence of SEQ ID NO: 13; b) HCDR2 comprising an amino acid sequence of SEQ ID NO: 14; c) HCDR3 comprising an amino acid sequence of SEQ ID NO: 15; d) LCDR1 comprising an amino acid sequence of SEQ ID NO: 18; e) LCDR2 comprising an amino acid sequence of SEQ ID NO: 19; and f) LCDR3 comprising an amino acid sequence of SEQ ID NO: 20; and wherein the recombinant bispecific antibody specifically binds to Her2/neu or p185HER2.

In some embodiments, the target antigen binding domain comprises: a) a VH sequence having at least 80% sequence identity to an amino acid sequence of SEQ ID NO: 12; and b) a VL sequence having at least 80% sequence identity to an amino acid sequence of SEQ ID NO: 17. In some embodiments, the target antigen binding domain comprises: a) a heavy chain sequence having at least 80% sequence identity to an amino acid sequence of SEQ ID NO: 11; and b) a light chain sequence having at least 80% sequence identity to an amino acid sequence of SEQ ID NO: 16.

In some embodiments, the target antigen binding domain comprises at least 80% sequence identity to the amino acid sequence between amino acid 20 and amino acid 110 of SEQ ID NO: 12 and at least 80% sequence identity to the amino acid sequence between amino acid 20 and amino acid 105 of SEQ ID NO: 17; and wherein the recombinant bispecific antibody specifically binds to Her2/neu or p185HER2.

### Effector Antigen Binding Domain

The recombinant bispecific antibodies comprise an effector antigen binding domain. The effector antigen binding domain specifically binds to a molecule present on an antigen presenting cell (APC), such as a dendritic cell. Antigen presenting cells regulate immune response by priming, and/or sustaining cell mediated immunity by T cells (both helper and cytotoxic T cells). As such, attracting antigen presenting cells to tumors and cancerous cells has the potential to boost cancer immunity and provide an adjuvant to monoclonal antibody therapy. This can be achieved by either providing activating signals to the APC (e.g., agonizing a costimulatory molecule) or blocking inhibitory signals (e.g., antagonizing a checkpoint inhibitor). In certain embodiments, the molecule present on the antigen presenting cell comprises a costimulatory molecule or other molecule that results in activation of the APC upon ligand binding. In certain embodiments, the costimulatory molecule is CD40, OX40L, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, or BDCA-2. In certain embodiments, the molecule present on the antigen presenting cell comprises a costimulatory molecule or other molecule that results in activation of the APC upon ligand binding. In certain embodiments, the costimulatory molecule is CD40, OX40L, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, BDCA-2, TNFR2, or TREM2. The effector antigen binding domain can be a ligand, agonist, or agonistic antibody that results in induction of one or more activation markers of the APC, for example, cytokine release, chemokine release, increased expression of cell surface molecules that engage T cells, including MHC class I, MHC class II or costimulatory molecules. Cytokine release can be measured by a cytokine release assay. Chemokine release can be measured by an ELISA immunoassay. Expression of cell surface molecules can be measured by FACS. In certain embodiments, the effector antigen binding domain is an agonist of CD40, CD80, CD86, OX40L, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, or BDCA-2. In certain embodiments, the effector antigen binding domain is an agonist of CD40, CD80, CD86, OX40L, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, BDCA-2, TNFR2, or TREM2. In certain embodiments, the effector antigen binding domain is TNFR2 or TREM. In certain embodiments, the effector antigen binding domain is an agonist of CD40. In certain embodiments, the effector antigen binding domain is derived from an agonist antibody of CD40, and possesses one or more CDR sequences derived from a CD40 agonistic antibody, such as, CP-870,893, APX005M, 3C3, 3G5, Dacetuzmumab and its non-fucosylated form SEA-CD40, or Chi Lob 7/4. The effector antigen binding domain can be a ligand, antagonist, or antagonistic antibody that antagonizes a checkpoint inhibitor and results in induction of one or more activation markers of the APC, for example, cytokine release, chemokine release, increased expression of cell surface molecules that engage T cells, including MHC class I, MHC class II or costimulatory molecules. Cytokine release can be measured by a cytokine release assay. Chemokine release can be measured by an ELISA immunoassay. Expression of cell surface molecules can be measured by FACS. In certain embodiments, the effector antigen binding domain is an antagonist of PD-L1, PD-L2, galectin-9, Indoleamine 2,3-dioxygenase, or CD276. In certain embodiments, the effector antigen binding domain may not be a lipocalin mutein. In certain embodiments, the effector antigen binding domain can be an antibody antigen binding domain.

Dendritic cells are key antigen presenting cells that participate in anti-cancer immunity. In certain embodiments, the effector antigen binding domain specifically binds to an antigen present on a dendritic cell. In certain embodiments, the antigen present on a dendritic cell comprises CD11b, CD11c, MHC class II molecules, CD40, CD80, CD86, OX40L, DEC-205, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, or BDCA-2. In certain embodiments, the effector antigen binding domain specifically binds to a protein with at least 80%, 90%, 95%, 97%, 98%, 99%, or 100% homology to CD11b, CD11c, MHC class II molecules, CD40, CD80, CD86, OX40L, DEC-205, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, or BDCA-2. In certain embodiments, the effector antigen binding domain specifically binds to a protein with at least 80%, 90%, 95%, 97%, 98%, 99%, or 100% homology to CD11b, CD11c, MHC class II molecules, CD40, CD80, CD86, OX40L, DEC-205, 4-1BBL, DEC-205, CD36, CD204, MARCO, DC-SIGN, CLEC9A, CLEC12A, CLEC5A, Dectin 1, Dectin 2, CLEC10A, CD206, CD4, CD32A, CD16A, HVEM, CD32B, PD-L1, BDCA-2, TNFR2, or TREM2. In certain embodiments, the effector antigen binding domain binds to the molecule present on the antigen presenting cell comprising at least 80%, 90%, 95%, 97%, 98%, 99%, or 100% homology to CD40, OX40L, DEC-205, 4-1BBL, CD36, CD204, MARCO, DC-SIGN, CLEC9A, CLEC5A, Dectin 2, CLEC10A, CD206, CD64, CD32A, CD16A, HVEM, CD32B, PD-L1, or BDCA-2. In certain embodiments, the effector antigen binding domain binds to the molecule present on the antigen presenting cell comprising at least 80%, 90%, 95%, 97%, 98%, 99%, or 100% homology to CD40, OX40L, DEC-205, 4-1BBL, CD36, CD204, MARCO, DC-SIGN, CLEC9A, CLEC5A, Dectin 2, CLEC10A, CD206, CD64, CD32A, CD16A, HVEM, CD32B, PD-L1, TNFR2, TREM2, or BDCA-2.

In some embodiments, the effector antigen binding domain of the recombinant bispecific antibody can have a different binding affinity for a molecule present on an antigen presenting cell in recombinant form compared to the binding affinity for the molecule present on the antigen presenting cell when the molecule is expressed by the antigen presenting cell. As used herein, the recombinant form of a molecule present on an antigen presenting cell (i.e., the recombinant form) can describe the molecule in a form that is independent from the antigen presenting cell, and therefore is not present on a cell or a live cell. In some aspects, a binding affinity of the effector antigen binding domain of the recombinant bispecific antibody to the molecule present on the antigen presenting cell is decreased compared to a binding affinity of the effector antigen binding domain of an antibody that lacks the target antigen binding domain, and this can occur even when the binding affinity of the effector antigen binding domain of the recombinant bispecific antibody to the molecule present on the antigen presenting cell in a recombinant form is similar to the binding affinity of the effector antigen binding domain of an antibody that lacks the target antigen binding domain. Furthermore, a Kd of the binding affinity of the effector antigen binding domain of the recombinant bispecific antibody to the molecule present on the antigen presenting cell can be increased by two times, five times, ten times, fifty times, or one-hundred times compared to the binding affinity of the effector antigen binding domain of an antibody that lacks the target antigen binding domain. In some aspects, the Kd for binding of the effector antigen binding domain to the molecule present on the antigen presenting cell is less than 20 nM, less than 100 nM, or less than 500 nM.

The effect of the target antigen binding domain and the effector antigen binding domain together can be to cluster antigen presenting cells around cancerous cells and at tumor sites resulting in activation of the antigen presenting effector functions of these cells. In certain embodiments, a recombinant bispecific antibody density of greater than 1000, 2000, 3000, 4000, 5000, 6000, 7000, 8000, 9000, 10,000 or more per cell, resulting from recombinant bispecific antibody binding to the tumor associated antigen, induces signaling in the antigen presenting cell. Signaling and clustering can suitably be measured in vitro. Signaling can be suitably measured using a cell line expressing the tumor associated antigen bound by the target antigen binding domain, and primary antigen presenting cells isolated from a human subject. Signaling can be assessed as cytokine release, chemokine release, or increased expression of cell surface markers. Cytokine release can be measured by a cytokine release assay. Chemokine release can be measured by an ELISA immunoassay. Expression of cell surface molecules can be measured by FACS.

In certain embodiments, the effector antigen binding domain of the recombinant bispecific antibody disclosed herein is derived from monoclonal antibody CP-870,893. CP-870,893 is a monoclonal antibody that specifically binds to and agonizes CD40. The heavy chain amino acid sequence for CP-870,893 is set forth in SEQ ID NO: 1; the light chain amino acid sequence for CP-870,893 is set forth in SEQ ID NO: 6. In certain embodiments, the effector antigen binding domain comprises a CDR sequence identified from either of SEQ ID NO: 1 or SEQ ID NO: 6 identified using the Kabat, IMGT, or Chothia method. In certain embodiments, the effector antigen binding domain comprises an amino acid sequence with at least 80%, 90%, 95%, 98%, 99%, or 100% identity to SEQ ID NO: 1. In certain embodiments, the effector antigen binding domain comprises an amino acid sequence with at least 80%, 90%, 95%, 98%, 99%, or 100% identity to SEQ ID NO: 6. In certain embodiments, the effector antigen binding domain comprises amino acid sequences with at least 80%, 90%, 95%, 98%, 99%, or 100% identity to SEQ ID NO: 1 and SEQ ID NO: 6.

The heavy chain variable region amino acid sequence for CP-870,893 is set forth in SEQ ID NO: 2; the light chain variable region amino acid sequence for CP-870,893 is set forth in SEQ ID NO: 7. In certain embodiments, the effector antigen binding domain comprises a CDR sequence identified from either of SEQ ID NOs: 2 or 7 identified using the Kabat, IMGT, or Chothia method. In certain embodiments, the effector antigen binding domain comprises an amino acid sequence with at least 80%, 90%, 95%, 98%, 99%, or 100% identity to SEQ ID NO: 2. In certain embodiments, the effector antigen binding domain comprises an amino acid sequence with at least 80%, 90%, 95%, 98%, 99%, or 100% identity to SEQ ID NO: 7. In certain embodiments, the effector antigen binding domain comprises an amino acid sequence with at least 80%, 90%, 95%, 98%, 99%, or 100% identity to SEQ ID NO: 2 and SEQ ID NO: 7.

In certain embodiments, the effector antigen binding domain comprises the CDR sequences of monoclonal antibody CP-870,893. The amino acid sequence of the HCDR1 of CP-870,893 is set forth in SEQ ID NO: 3; the amino acid sequence of the HCDR2 of CP-870,893 is set forth in SEQ ID NO: 4; the amino acid sequence of the HCDR3 of CP-870,893 is set forth in SEQ ID NO: 5; the amino acid sequence of the LCDR1 of CP-870,893 is set forth in SEQ ID NO: 8; the amino acid sequence of the LCDR2 of CP-870,893 is set forth in SEQ ID NO: 9; and the amino acid sequence of the LCDR3 of CP-870,893 is set forth in SEQ ID NO: 10. In certain embodiments, the effector antigen binding domain comprises any one of SEQ ID NO: 3-SEQ ID NO: 5 and SEQ ID NO: 8-SEQ ID NO: 10. In certain embodiments, the effector antigen binding domain comprises all of SEQ ID NO: 3-SEQ ID NO: 5 and SEQ ID NO: 8-SEQ ID NO: 10. In certain embodiments, the effector antigen binding domain comprises any one of SEQ ID NO: 3-SEQ ID NO: 5. In certain embodiments, the effector antigen binding domain comprises any one of SEQ ID NO: 8-SEQ ID NO: 10. In certain embodiments, the effector antigen binding domain comprises all of SEQ IDO NO: 3-SEQ ID NO: 5. In certain embodiments, the effector antigen binding domain comprises all of SEQ ID NO: 8-SEQ ID NO: 10. The CDR sequences above may be incorporated into the effector antigen binding domain by any method of recombinant DNA technology. The incorporated CDRs can have any amount of amino acid sequence identity to any one of SEQ ID NO: 3-SEQ ID NO: 5 and SEQ ID NO: 8-SEQ ID NO: 10 that still retains the specific binding of CP-870,893, for example, 80%, 90%, 95%, 98%, 99%, or 100% identity.

In certain embodiments, the effector antigen binding domain comprises a CD40 scFv. The CD40 scFv can be either attached (fused via a peptide bond between amino acids) or coupled (via a linker) to the C-terminus of the Fc comprising domain polypeptide. In certain embodiments, the CD40 scFv comprises an amino acid sequence set forth in SEQ ID NO: 1312. In certain embodiments, the effector antigen binding domain attached to the Fc comprising domain comprises an amino acid sequence at least 80%, 90%, 95%, 98%, 99%, or 100% identical to that set forth in SEQ ID NO: 1312.

In certain embodiments, the recombinant bispecific antibody comprises an effector antigen binding with at least 80%, 90%, 95%, 98%, 99%, or 100% sequence identity to 6, 7, 8, 9, 10, 11, 12 or more contiguous amino acids between amino acid 20 and amino acid 110 of SEQ ID NO: 12 and at least 80%, 90%, 95%, 98%, 99%, or 100% sequence identity to 6, 7, 8, 9, 10, 11, 12 or more contiguous amino acids between amino acid 20 and amino acid 105 of SEQ ID NO: 17.

In some embodiments, the effector antigen binding domain comprises an immunoglobulin heavy chain variable region or antigen binding fragment thereof and an immunoglobulin light chain variable region or antigen binding fragment thereof. In some embodiments, the effector antigen binding domain comprises a single chain variable region fragment (scFv).

In some embodiments, the scFv comprises at least 80% sequence identity to an amino acid sequence of SEQ ID NO: 1312.

In some embodiments, the antigen presenting cell is a dendritic cell. In some embodiments, the antigen on the antigen presenting cell is a costimulatory molecule.

In some embodiments, the antigen on the antigen presenting cell is selected from the group consisting of CD40, OX40L, DEC-205, 4-1BBL, CD36, CD204, MARCO, DC-SIGN, CLEC9A, CLEC5A, Dectin 2, CLEC10A, CD206, CD64, CD32A, CD1A, HVEM, CD32B, PD-L1, or BDCA-2. In some embodiments, the effector antigen binding domain is a CD40 agonist.

In some embodiments, the effector antigen binding domain comprises the following CDRs: a) HCDR1 comprising an amino acid sequence of SEQ ID NO: 3; b) HCDR2 comprising an amino acid sequence of SEQ ID NO: 4; c) HCDR3 comprising an amino acid sequence of SEQ ID NO: 5; d) LCDR1 comprising an amino acid sequence of SEQ ID NO: 8; e) LCDR2 comprising an amino acid sequence of SEQ ID NO: 9; and f) LCDR3 comprising an amino acid sequence of SEQ ID NO: 10.

In some embodiments, the effector antigen binding domain comprises: a) a V_{H} sequence having at least 80% sequence identity to an amino acid sequence of SEQ ID NO: 2; and b) a VL sequence having at least 80% sequence identity to an amino acid sequence of SEQ ID NO: 7. In some embodiments, the effector antigen binding domain comprises: a) a heavy chain sequence having at least 80% sequence identity to an amino acid sequence of SEQ ID NO: 1; and b) a light chain having at least 80% sequence identity to an amino acid sequence of SEQ ID NO: 6.

In some embodiments, the antigen on the antigen presenting cell is TREM2 or TNFR2.

In some embodiments, the effector antigen binding domain has an increased binding affinity to the antigen on the antigen presenting cell as compared to the binding affinity of the effector antigen binding domain of an antibody that lacks the target antigen binding domain.

In some embodiments, a K_{d} of the binding affinity of the effector antigen binding domain of the recombinant bispecific antibody to the antigen on the antigen presenting cell is increased by two times, five times, ten times, fifty times, or one-hundred times compared to the binding affinity of the effector antigen binding domain of an antibody that lacks the target antigen binding domain.

In some embodiments, a K_{d} for binding of the effector antigen binding domain to the antigen on the antigen presenting cell is less than 20 nM, less than 100 nM, or less than 500 nM.

### Nucleotides Encoding Antibodies

In certain embodiments, antibodies are encoded by polynucleotides for expression and purification from a cell based system. Suitable polynucleotides include vectors such as DNA plasmids, viral vectors, and RNA molecules. Suitable viral vectors include retroviral, lentiviral, adenoviral, or baculoviral vectors.

Antibodies can be produced by a suitable method including by synthesis, in a cell based system, or a combination thereof. A suitable cell line for the production of the recombinant antibodies includes the CHO (Chinese hamster ovary) cell line or variants/derivatives thereof. Other suitable cell lines include AGE1.HN, NS0, Sp2/0, BHK21, HEK-293, HT-1080, PER.C6, HKB-11, CAP, and HuH-7 cell lines or variants/derivatives thereof. In a certain embodiment, the cell(s) utilized for production is transiently transfected or infected with the vector(s). In a certain embodiment, the production cell(s) is stably transfected with the vector(s), and constitutes a master cell bank for the production of antibodies. A master cell bank allows freezing and preservation of an antibody producing cell line. This allows for more efficient and consistent production of antibodies. Cells that are transfected or infected with vector(s) encoding an antibody are then cultured in a growth media for at least 1, 3, 5, 7, 9, 11, 14 days or more and the growth media is harvested for purification of the antibody. In certain embodiments, the growth media lacks serum of human or animal origin.

In certain embodiments, antibodies isolated or purified after secretion from a cell based system. Purification includes at least one step comprising centrifugation, precipitation, filtration, dialysis. The Fc region of the antibodies may retain the ability to interact with bacterial super antigens such as Protein A or Protein G. In certain embodiments, purification comprises a step utilizing Protein A, Protein G, or a combination thereof, to specifically separate recombinant antibodies from other secreted proteins and serum. In certain embodiments, column chromatography is utilized.

### Immune-Stimulatory Compounds

The antibody constructs and recombinant bispecific antibodies described herein can further be attached to an immune-stimulatory compound to form a conjugate. The immune-stimulatory compound can provide a direct, indirect or adjuvant effect. In certain embodiments, the immune-stimulatory compound can be coupled to the antibody construct, such as to the Fc domain of the antibody construct. An immune-stimulatory compound can be any compound that directly or indirectly stimulates an anti-tumor immune response after administration. For example, an immune-stimulatory compound can directly stimulate an anti-tumor immune response by causing the release of cytokines by its target cell, which results in the activation of immune cells. As another example, an immune-stimulatory compound can indirectly stimulate an immune response by suppressing IL-10 production and secretion by the target cell and/or by supressing the activity of regulatory T cells, resulting in an increased anti-tumor response by immune cells. The stimulation of an immune response by an immune-stimulatory compound can be measured by the upregulation of proinflammatory cytokines and/or increased activation of immune cells. This effect can be measured in vitro by co-culturing immune cells with cells targeted by the immune-stimulatory conjugate and measuring cytokine release, chemokine release, proliferation of immune cells, upregulation of immune cell activation markers, and/or ADCC. ADCC can be measured by an ADCC assay, which can determine the percentage of remaining target cells, such as tumor cells, in the co-culture after administration of the immune-stimulatory conjugate with the target cells and immune cells.

In certain embodiments, an immune-stimulatory compound can target a pattern recognition receptor (PRR). PRRs can recognize pathogen-associated molecular patterns (PAMPs) and damage-associated molecular patterns (DAMPs). A PRR can be membrane bound. A PRR can be cytosolic. A PRR can be a toll-like receptor (TLR). A PRR can be RIG-I-like receptor. A PRR can be a receptor kinase. A PRR can be a C-type lectin receptor. A PRR can be a NOD-like receptor. A PRR can be TLR1, TLR2, TLR3, TLR4, TLR5, TLR6, TLR7, TLR8, TLR9, TLR10, TLR11, TLR12 or TLR13. A PRR can be TLR1, TLR2, TLR3, TLR4, TLR5, TLR6, TLR7, TLR8, TLR9, and TLR10.

In certain embodiments, the immune-stimulatory compound can be a Damage-Associated Pattern Molecule (DAMP) or a Pathogen-Associated Molecular Pattern Molecule (PAMP). Immune-stimulatory molecular motifs, such as PAMPs, can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens. Similar to other protein families, TLRs can have many isoforms, including TLR4, TLR7 and TLR8. TLR agonists can range from simple molecules to complex macromolecules. Likewise, the sizes of TLR agonists can range from small to large. TLR agonists can be synthetic or biosynthetic agonists. TLR agonists can also be PAMPs. Additional immune-stimulatory compounds, such as cytosolic DNA and unique bacterial nucleic acids called cyclic dinucleotides, can be recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

Imiquimod, a synthetic TLR7 agonist, is currently approved for human therapeutic applications. Contained in a cream and marketed under the brand name Aldara™, imiquimod serves as a topical treatment for a variety of indications with immune components, such as, actinic keratosis, genital warts, and basal cell carcinomas. In addition, imiquimod is indicated as a candidate adjuvant for enhancing adaptive immune responses when applied topically at an immunization site.

Another type of immune stimulatory molecular motif, Damage-Associated Molecular Mattern molecules (DAMPs), can initiate and maintain an immune response occurring as part of the non-infectious inflammatory response. DAMPs can be specially localized proteins that, when detected by the immune system in a location other than where DAMPs should be located, activate the immune system. Often, DAMPs can be nuclear or cytosolic proteins and upon release from the nucleus or cytosol, DAMP proteins can become denatured through oxidation. Examples of DAMP proteins can include chromatin-associated protein high-mobility group box 1 (HMGB 1), S100 molecules of the calcium modulated family of proteins and also glycans, such as hyaluronan fragments, and glycan conjugates. DAMPs can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

In certain embodiments, an immune-stimulatory compound can be a Toll-like receptor agonist, a STING agonist, or a RIG-I agonist.

In certain embodiments, an immune-stimulatory compound can be a TLR agonist. Additionally, the immune response elicited by TLR agonists can further be enhanced when co-administered with a CD40-agonist antibody. For example, co-administration of a TLR agonist such as poly IC:LC with a CD40-agonist antibody can synergize to stimulate a greater CD8+ T cell response than either agonist alone.

In certain embodiments, the immune-stimulatory compound can be S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663.

In certain embodiments, the immune-stimulatory compound can be a TLR4 agonist, such as AZ126 (N-(2-(cyclopentylamino)-2-oxo-1-(pyridin-4-yl)ethyl)-N-(4-methoxyphenyl)-3-methyl-5-phenyl-1H-pyrrole-2-carboxamide) or AZ368 ((E)-3-(4-(2-(cyclopentylamino)-1-(N-(4-isopropylphenyl)-1,5-diphenyl-1H-pyrazole-3-carboxamido)-2-oxoethyl)phenyl)acrylic acid).

In some embodiments, the immune-stimulatory compound can be a TLR7 agonist, such as TLR7 agonist R848.

The immune-stimulatory compound can comprise an inhibitor of TGFB, Beta-Catenin, PI3K-beta, STAT3, IL-10, IDO or TDO. The immune-stimulatory compound can be an inhibitor of the beta-catenin pathway, such as an inhibitor of TNIK or Tankyrase. In certain embodiments, the immune-stimulatory compound be a kinase inhibitor. In certain embodiments, the kinase inhibitor can be an inhibitor of CDK4/6, such as, for example, abemaciclib or palbociclib.

The immune-stimulatory compound can be LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843.

In certain embodiments, the immune-stimulatory compound can be coupled to the antibody construct via a linker. In certain embodiments, the immune-stimulatory compound is coupled to the antibody construct using a linker wherein the immune-stimulatory compound comprises a Toll-like receptor agonist, STING agonist, or RIG-I agonist. In certain embodiments, the Toll-like receptor agonist comprises a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, or a peptidoglycan. In certain embodiments, the STING agonist comprises a cyclic dinucleotide. In certain embodiments, the RIG-1 agonist comprises a 5′ppp-dsRNA.

A PRR agonist can be pathogen-associated molecular pattern (PAMP) molecule. A PAMP molecule can be a toll-like receptor agonist. A PRR agonist can be a toll-like receptor agonist. A toll-like receptor agonist can be any molecule that acts as an agonist to at least one toll-like receptor. A toll-like receptor agonist can be bacterial lipoprotein. A toll-like receptor agonist can be bacterial peptidoglycans. A toll-like receptor agonist can be double stranded RNA. A toll-like receptor agonist can be lipopolysaccharides. A toll-like receptor agonist can be bacterial flagella. A toll-like receptor agonist can be single stranded RNA. A toll-like receptor can be CpG DNA. A toll-like receptor agonist can be imiquimod. A toll-like receptor agonist can be CL307. A toll-like receptor agonist can be S-27609. A toll-like receptor agonist can be resiquimod. A toll-like receptor agonist can be UC-IV150. A toll-like receptor agonist can be gardiquimod. A toll-like receptor agonist can be motolimod. A toll-like receptor agonist can be VTX-1463. A toll-like receptor agonist can be GS-9620. A toll-like receptor agonist can be GSK2245035. A toll-like receptor agonist can be TMX-101. A toll-like receptor agonist can be TMX-201. A toll-like receptor agonist can be TMX-202. A toll-like receptor agonist can be isatoribine. A toll-like receptor agonist can be AZD8848. A toll-like receptor agonist can be MEDI9197. A toll-like receptor agonist can be 3M-051. A toll-like receptor agonist can be 3M-852. A toll-like receptor agonist can be 3M-052. A toll-like receptor agonist can be 3M-854A. A toll-like receptor agonist can be S-34240. A toll-like receptor agonist can be CL663. A RIG-I agonist can be KIN1148. A RIG-I agonist can be SB-9200. A RIG-I agonist can be KIN700, KIN600, KIN500, KIN100, KIN101, KIN400, or KIN2000. A toll-like receptor agonist can be KU34B.

A PRR agonist can be a damage-associated molecular pattern (DAMP) molecule. A DAMP molecule can be an intracellular protein. A DAMP molecule can be a heat-shock protein. A DAMP molecule can be an HMGB 1 protein. A DAMP molecule can be a protein derived from the extracellular matrix that is generated after tissue injury. A DAMP molecule can be a hyaluronan fragment. A DAMP molecule can be DNA. A DAMP molecule can be RNA. A DAMP molecule can be an S100 molecule. A DAMP molecule can be nucleotides. A DAMP molecule can be an ATP. A DAMP molecule can be nucleosides. A DAMP molecule can be an adenosine. A DAMP molecule can be uric acid.

Additionally, an immune-stimulatory compound can target stimulator of interferon genes (STING). STING can act as a cytosolic DNA sensor wherein cytosolic DNA and unique bacterial nucleic acids called cyclic dinucleotides are recognized by STING, and therefore STING agonists. Interferon Regulatory Factor (IRF) agonist can be KIN-100. Non-limiting examples of STING agonists include:

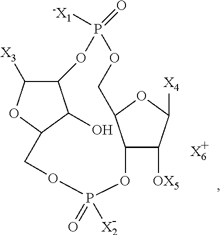

wherein in some embodiments, X₁═X₂═O; X₃=G; X₄=G; X₅═CO(CH₂)₁₂CH₃; X₆=2 TEAH; in some embodiments, X₁═X₂═S [Rₚ,Rₚ]; X₃=G; X₄=A; X₅═H; X₆=2 TEAH; in some embodiments, X₁═X₂═S [Rₚ,Rₚ]; X₃=A; X₄=A; X₅═H; X₆=2 Na; in some embodiments, X₁═X₂═S [Rₚ,Rₚ]; X₃=A; X₄=A; X₅═H; X₆=2 NH₄; and in some embodiments, X₁═X₂═O; X₃=G; X₄=A; X₅═H; X₆=2 TEAH,

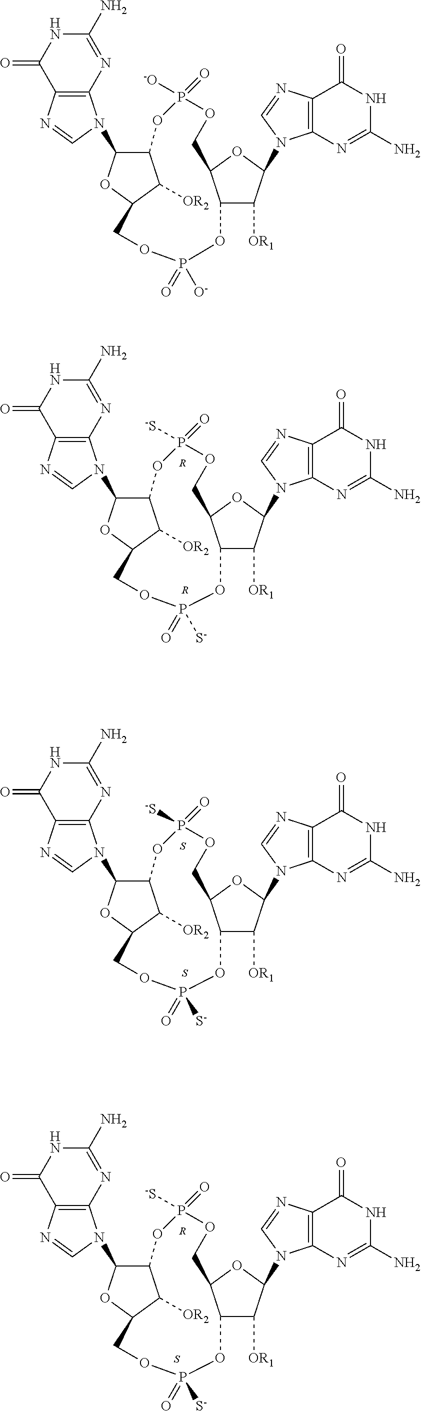

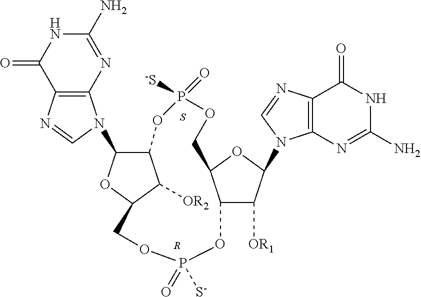

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

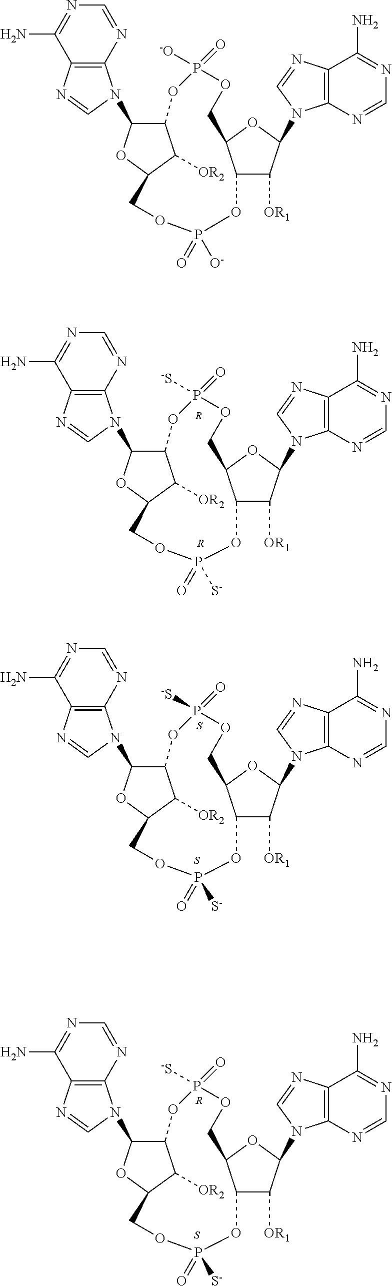

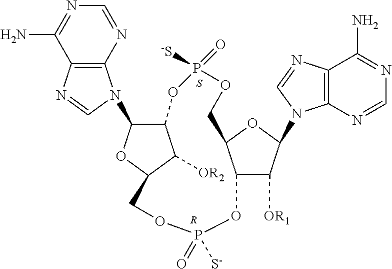

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

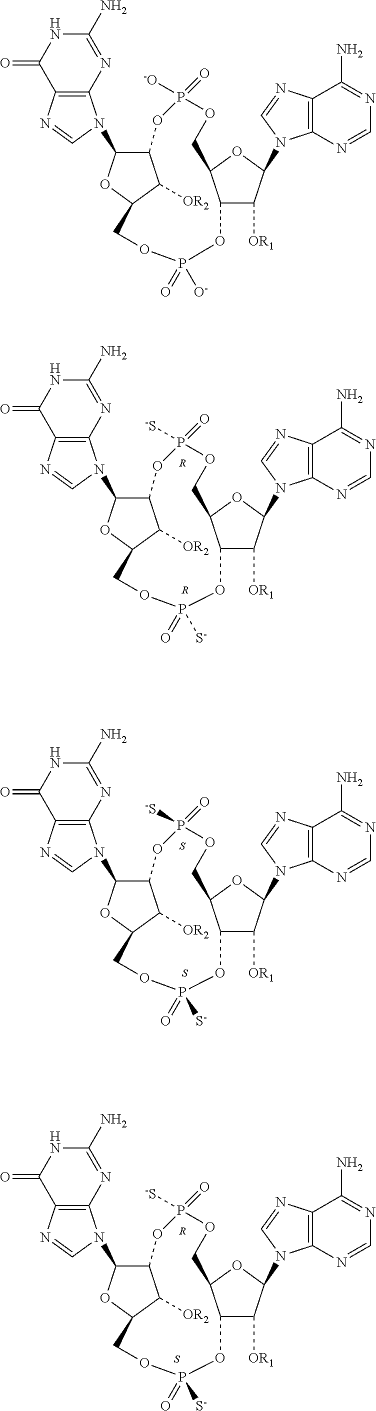

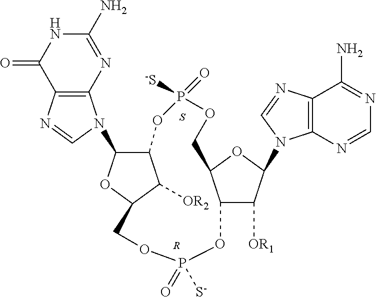

wherein R₁═R₂═H; R₁=propargyl; R₂═H; R₁═H, R₂=propargyl; R₁=allyl, R₂═H; R₁═H, R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

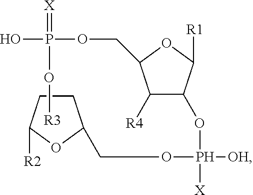

wherein each X is independently O or S, and R3 and R4 are each independently H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, OH, —O, —COOR′— where R′ is H or lower alkyl, —CH₂OH, and —CONH₂, wherein R3 and R4 are not both H,

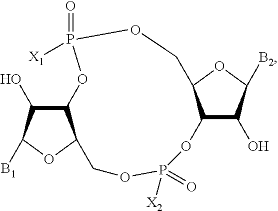

wherein X₁═X₂═O; X₁═X₂═S; or X₁═O and X₂═S,

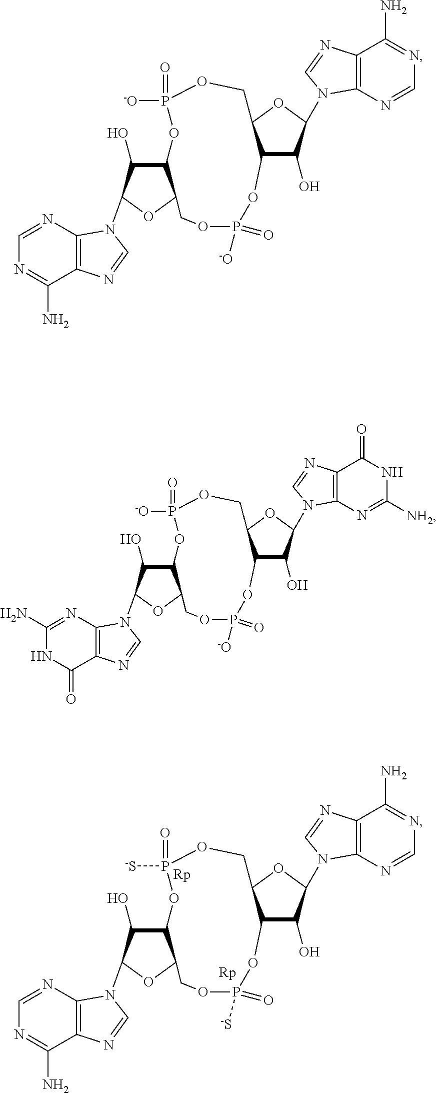

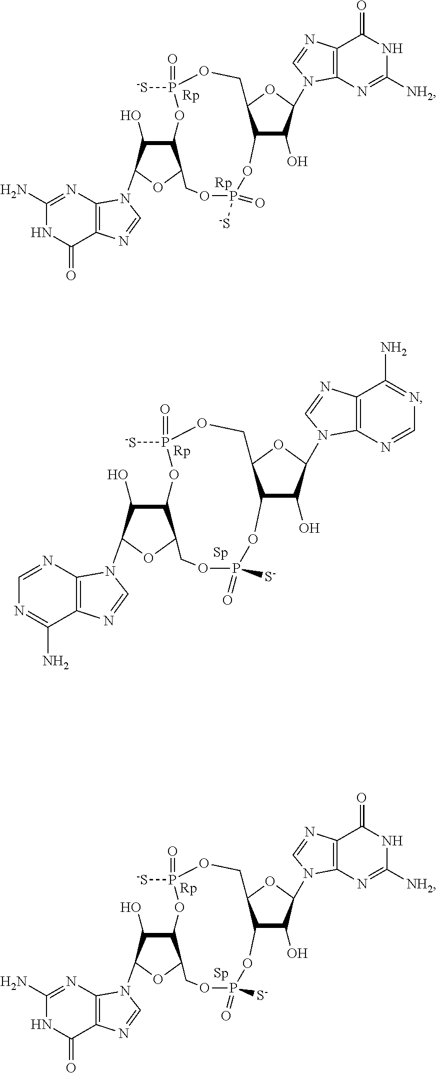

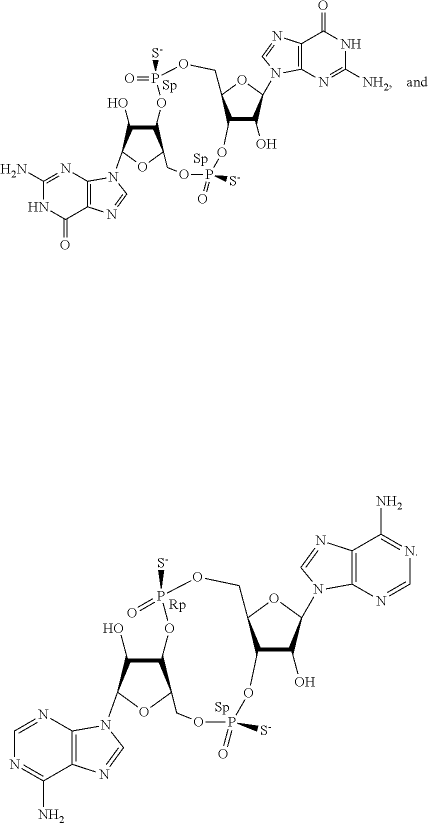

An immune-stimulatory compound can be a PRR agonist. An immune-stimulatory compound can be a PAMP. An immune-stimulatory compound can be a DAMP. An immune-stimulatory compound can be a TLR agonist. An immune-stimulatory compound can be a STING agonist. An immune-stimulatory compound can be a cyclic dinucleotide.

The specificity of the antigen-binding domain to an antigen of a conjugate disclosed herein can be influenced by the presence of an immune-stimulatory compound. The antigen-binding domain of the conjugate can bind to an antigen with at least about 10%, about 20%, about 30%, about 40%, about 50%, about 60%, about 70%, about 80%, about 85%, about 90%, about 95%, or about 100% of a specificity of the antigen-binding domain to the antigen in the absence of the immune-stimulatory compound.

The specificity of the Fc domain to an Fc receptor of a conjugate disclosed herein can be influenced by the presence of an immune-stimulatory compound. The Fc domain of the conjugate can bind to an Fc receptor with at least about 10%, about 20%, about 30%, about 40%, about 50%, about 60%, about 70%, about 80%, about 85%, about 90%, about 95%, or about 100% of a specificity of the Fc domain to the Fc receptor in the absence of the immune-stimulatory compound.

The affinity of the antigen-binding domain to an antigen of a conjugate disclosed herein can be influenced by the presence of an immune-stimulatory compound. The antigen-binding domain of the conjugate can bind to an antigen with at least about 1%, about 5%, about 10%, about 20%, about 30%, about 40%, about 50%, about 60%, about 70%, about 80%, about 85%, about 90%, about 95%, or about 100% of an affinity of the antigen-binding domain to the antigen in the absence of the immune-stimulatory compound.

The affinity of the Fc domain to an Fc receptor of a conjugate disclosed herein can be influenced by the presence of an immune-stimulatory compound. The Fc domain of the conjugate can bind to an Fc receptor with at least about 1%, about 5%, about 10%, about 20%, about 30%, about 40%, about 50%, about 60%, about 70%, about 80%, about 85%, about 90%, about 95%, or about 100% of an affinity of the Fc domain to the Fc receptor in the absence of the immune-stimulatory compound.

The K_{d} for binding of an antigen-binding domain to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the immune-stimulatory compound.

The K_{d} for binding of an Fc domain to a Fc receptor in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulator compound.

Affinity can be the strength of the sum total of noncovalent interactions between a single binding site of a molecule, for example, an antibody, and the binding partner of the molecule, for example, an antigen. The affinity can also measure the strength of an interaction between an Fc portion of an antibody or antibody construct and the Fc receptor. Unless indicated otherwise, as used herein, “binding affinity” can refer to intrinsic binding affinity which reflects a 1:1 interaction between members of a binding pair (e.g., antibody and antigen or Fc domain and Fc receptor). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

In some embodiments, an antibody or antibody construct provided herein can have a dissociation constant (K_{d}) of about 1 μM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸ M or less, e.g., from 10⁻⁸ M to 10⁻¹³ M, e.g., from 10⁻⁹ M to 10⁻¹³ M). An affinity matured antibody can be an antibody with one or more alterations in one or more complementarity determining regions (CDRs), compared to a parent antibody, which may not possess such alterations, such alterations resulting in an improvement in the affinity of the antibody for antigen. These antibodies can bind to their antigen with a K_{d} of about 5×10⁻⁹ M, about 2×10⁻⁹ M, about 1×10⁻⁹ M, about 5×10⁻¹ M, about 2×10⁻⁹ M, about 1×10⁻¹⁰ M, about 5×10−11 M, about 1×10¹¹ M, about 5×10⁻¹² M, about 1×10⁻¹² M, or less. In some embodiments, the conjugate can have an increased affinity of at least 1.5-fold, 2-fold, 2.5-fold, 3-fold, 4-fold, 5-fold, 10-fold, 20-fold, or greater as compared to a conjugate without alterations in one or more complementarity determining regions.

K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000).

Agonism can be described as the binding of a chemical to a receptor to induce a biological response. A chemical can be, for example, a small molecule, a compound, or a protein. An agonist causes a response, an antagonist can block the action of an agonist, and an inverse agonist can cause a response that is opposite to that of the agonist. A receptor can be activated by either endogenous or exogenous agonists.

The molar ratio of a conjugate refers to the average number of immune-stimulatory compounds conjugated to the antibody construct in a preparation of a conjugate. The molar ratio can be determined, for example, by Liquid Chromatography/Mass Spectrometry (LC/MS), in which the number of immune-stimulatory compounds conjugated to the antibody construct can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of immune-stimulatory compound to antibody construct can be less than 8. In other embodiments, the molar ratio of immune-stimulatory compound to antibody construct can be 8, 7, 6, 5, 4, 3, 2, or 1.

In some aspects, the present disclosure provides an immune-stimulatory compound represented by the structure of Formula (I):

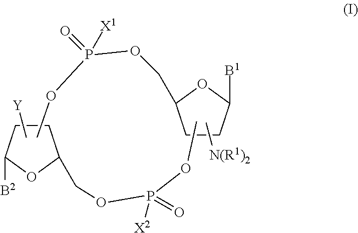

or a pharmaceutically acceptable salt thereof, wherein:
X¹ is selected from —OR² and —SR²;
X² is selected from —OR³ and —SR³;
B¹ and B² are independently selected from optionally substituted nitrogenous bases;
Y is selected from —OR⁴, —NR⁴R⁴, and halogen;

R¹, R², R³ and R⁴ are independently selected at each occurrence from hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³ and R⁴ is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl; and R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl.

In some embodiments, the compound of Formula (I) is represented by Formula (IA):

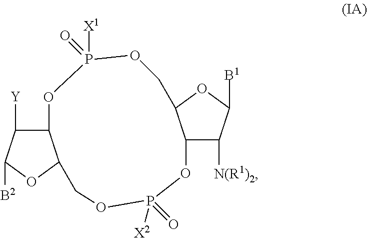

or pharmaceutically acceptable salts thereof.

In an alternative embodiment, the compound of Formula (I) is represented by Formula (IB):

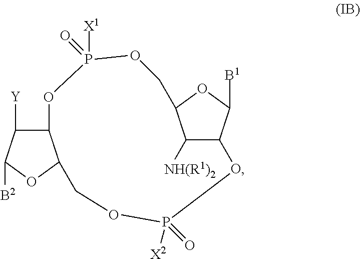

or a pharmaceutically acceptable salt thereof.

In various embodiments, B¹ and B² are independently selected from optionally substituted purines. In certain embodiments, B¹ and B² are independently selected from:

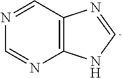

In certain embodiments, B¹ and B² are independently selected from optionally substituted pyrimidines.

In some embodiments, optionally substituted purines may include optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In certain embodiments, B¹ and B² are independently selected from:

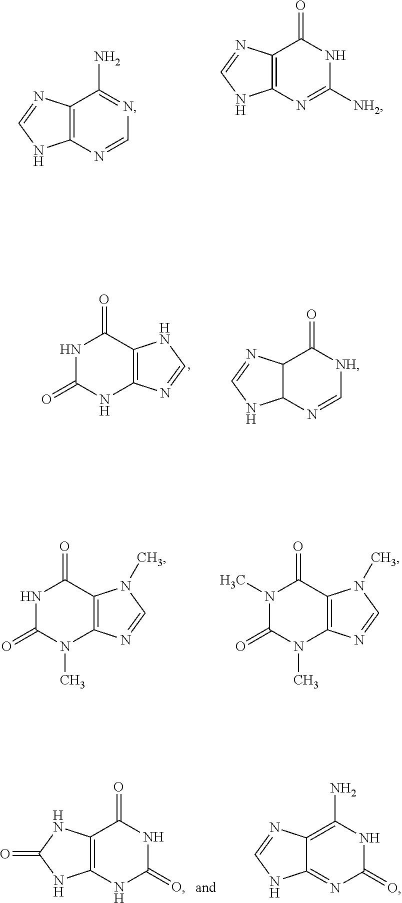

optionally substituted by one or more additional substituents.

In certain embodiments, B¹ and B² are independently selected from:

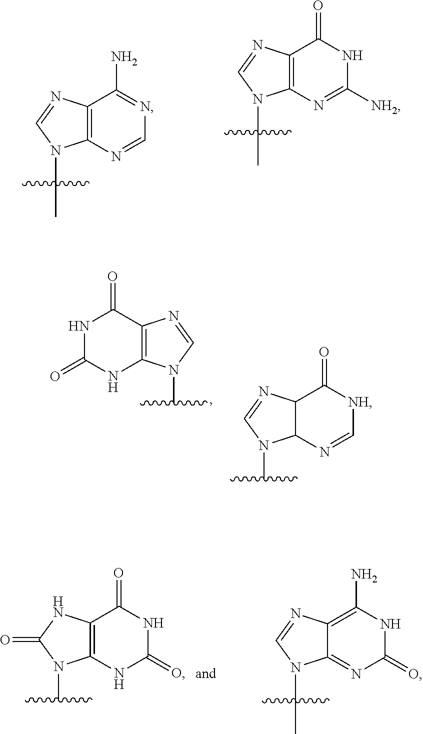

wherein the point of connectivity of B to the remainder of the compound is represented by

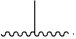

In a preferred embodiment, B¹ and B² are independently selected from optionally substituted adenine and optionally substituted guanine. In certain embodiments, B¹ and B² are independently selected from

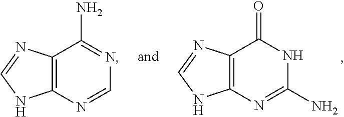 [SMILES: Nc1ncnc2[nH]cnc12 ; Nc1nc2[nH]cnc2c(=O)[nH]1]

optionally further substituted by one or more substituents. In certain embodiments, B¹ and B² are independently selected from:

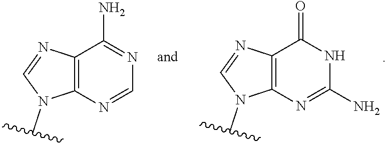 [SMILES: *n1cnc2c(N)ncnc21 ; *n1cnc2c(=O)[nH]c(N)nc21]

In some embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂ and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, — —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl.

In certain embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂, —CN and C₁₋₁₀ alkyl.

In some embodiments, B¹ is an optionally substituted guanine. In certain embodiments, B¹ is

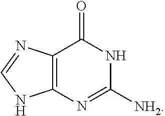 [SMILES: Nc1nc2[nH]cnc2c(=O)[nH]1]

In certain embodiments, B¹ is

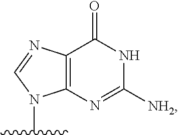 [SMILES: *n1cnc2c(=O)[nH]c(N)nc21]

wherein the point of connectivity of B1 to the remainder of the compound is represented by

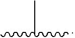

In some embodiments, B¹ is an optionally substituted adenine. In certain embodiments, B¹ is

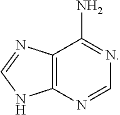

In certain embodiments, B¹ is

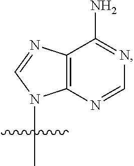 [SMILES: *n1cnc2c(N)ncnc21]

wherein the point of connectivity of B¹ to the remainder of the compound is represented by

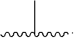

In some embodiments, B² is an optionally substituted guanine. In certain, embodiments, B² is

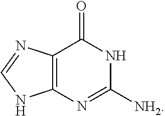 [SMILES: Nc1nc2[nH]cnc2c(=O)[nH]1]

In certain embodiments, B² is

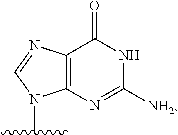 [SMILES: *n1cnc2c(=O)[nH]c(N)nc21]

wherein the point of connectivity on B² is represented by

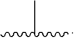

In some embodiments, B² is an optionally substituted adenine. In certain embodiments, B² is

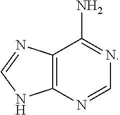

In certain embodiments, B² is

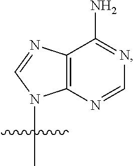 [SMILES: *n1cnc2c(N)ncnc21]

wherein the point of connectivity on B² is represented by

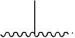

In some embodiments, B¹ is an optionally substituted guanine and B² is an optionally substituted guanine. In some embodiments, B¹ is an optionally substituted adenine and B² is an optionally substituted guanine.

In various embodiments, X¹ is selected from —OH and —SH. For example, X¹ may be —OH. In various embodiments, X² is selected from —OH and —SH. For example, X² may be —OH. In some embodiments, X¹ is —OH and X² is —OH. In some embodiments, X¹ is —SH and X² is —SH.

In various embodiments, Y is selected from —OH, —O—C₁₋₁₀ alkyl, —NH(C₁₋₁₀ alkyl), and —NH₂. For example, Y may be —OH.

In various embodiments, R¹⁰⁰ is independently selected at each occurrence from hydrogen and C₁₋₁₀ alkyl optionally substituted at each occurrence with one or more substituents selected from halogen, —CN, —NO₂, ═O, and ═S.

In various embodiments, the compound of Formula (I) is represented by Formula (IC):

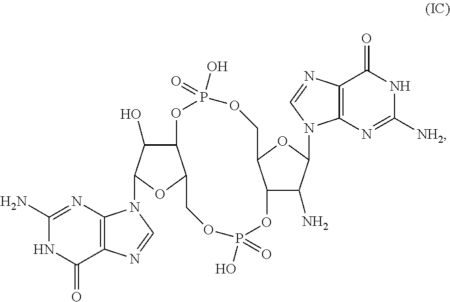 [SMILES: Nc1nc2c(ncn2C2OC3COP(=O)(O)OC4C(COP(=O)(O)OC3C2N)OC(n2cnc3c(=O)[nH]c(N)nc32)C4O)c(=O)[nH]1]

or a pharmaceutically acceptable salt thereof. In some embodiments, the compound of Formula (IC) is represented by Formula (ID):

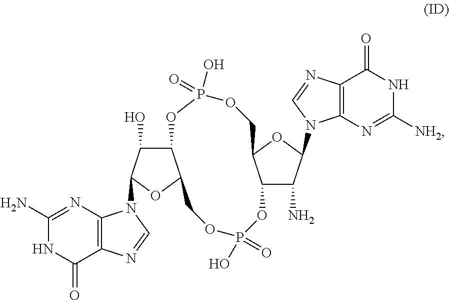 [SMILES: Nc1nc2c(ncn2[C@@H]2O[C@@H]3COP(=O)(O)O[C@H]4[C@@H](O)[C@H](n5cnc6c(=O)[nH]c(N)nc65)O[C@@H]4COP(=O)(O)O[C@H]3[C@H]2N)c(=O)[nH]1]

or a pharmaceutically acceptable salt thereof.

In various embodiments, the compound is a pharmaceutically acceptable salt. In some embodiments, the compound or salt is a modulator of a stimulator of interferon genes (STING). The compound or salt may agonize a stimulator of interferon genes (STING). In certain embodiments, the compound or salt may cause STING to coordinate multiple immune responses to infection, including the induction of interferons and STAT6-dependent response and selective autophagy response. In certain embodiments, the compound or salt may cause STING to mediate type I interferon production.

In some aspects, the present disclosure provides a compound represented by the structure of Formula (II):

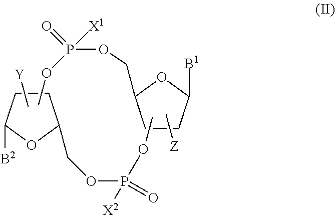

or a pharmaceutically acceptable salt thereof, wherein:

X¹ is selected from —OR² and —SR²;

X² is selected from —OR³ and —SR³;
B¹ and B² are independently selected from optionally substituted nitrogenous bases, wherein each optional substituent is independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, R⁶, and —X³;

Y is selected from —OR⁴, —SR⁴, —NR⁴R⁴, and halogen;

Z is selected from —OR⁵, —SR⁵, and —NR⁵R⁵;
R¹, R², R³, R⁴, and R⁵ are independently selected from a —X³; hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³, R⁴, and R⁵ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl;
R⁶ is independently selected from —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R⁶ is optionally substituted with one or more substituents selected from halogen, —OR¹⁰⁰, —SR¹⁰⁰, —N(R)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl;

R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl; and

X³ is a linker moiety, wherein at least one of R¹, R², R³, R⁴, R⁵, X¹, X², a B¹ substituent and a B² substituent is —X³.

In various embodiments, the compound of Formula (II) is represented by a structure of Formula (IIA):

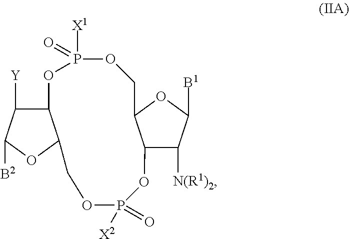

or pharmaceutically acceptable salts thereof.

In various embodiments, the compound of Formula (II) is represented by a structure of Formula (IIB):

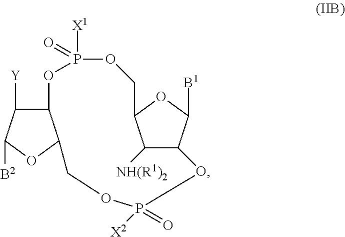

or a pharmaceutically acceptable salt thereof.

In various embodiments, B¹ and B² are independently selected from optionally substituted purines. B¹ and B² may be each, independently selected from one another, adenine, guanine, and derivatives thereof. B¹ and B² may be independently selected from optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In a preferred embodiment, B¹ and B² are independently selected from optionally substituted adenine and optionally substituted guanine.

In various embodiments, B¹ is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B¹ may be represented by:

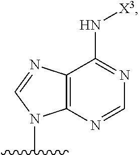

and wherein B¹ is optionally further substituted by one or more substituents.

In various embodiments, B is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B² may be represented by:

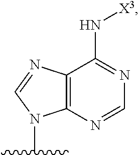

and wherein B² is optionally further substituted by one or more substituents.

In some embodiments, B¹ is represented by

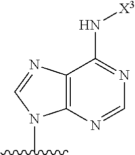

and B² is represented by

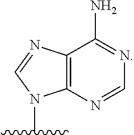 [SMILES: *n1cnc2c(N)ncnc21]

In some embodiments, B¹ is represented by

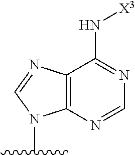

and B² is represented by

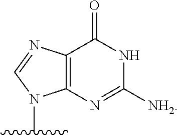 [SMILES: *n1cnc2c(=O)[nH]c(N)nc21]

In various embodiments, X¹ is selected from —O—X³ and —S—X³. In some embodiments, X¹ is selected from —OH and —SH. In some embodiments, X¹ is —SH.

In various embodiments, X² is selected from —O—X³ and —S—X³. In some embodiments, X² is selected from —OH and —SH. In some embodiments, X² is —S—X³.

In some embodiments, X¹ is —SH and X² is —S—X³.

In certain embodiments, Y is selected from —NR⁴X³, —S—X³, and —O—X³. In some embodiments, Y is selected from —OH, —SH, —O—C₁₋₁₀ alkyl, —NH(C₁₋₁₀ alkyl), and —NH₂. In a preferred embodiment, Y is selected from —OH.

In various embodiments, Z is selected from —NR⁴X³, —S—X³, and —O—X³. In some embodiments, Z is selected from —OH, —SH, —O—C₁₋₁₀ alkyl, —NH(C₁₋₁₀ alkyl), and —NH₂.

In various embodiments, —X³ is represented by the formula:

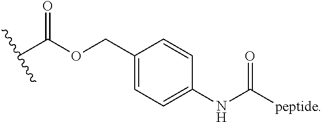

In some embodiments, —X³ is represented by the formula:

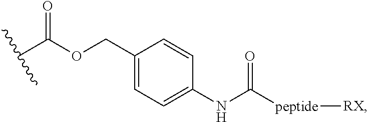

wherein RX comprises a reactive moiety, such a maleimide.

In some embodiments, —X³ is represented by the formula:

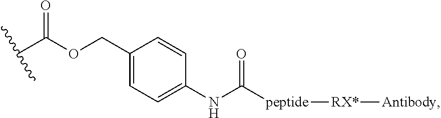

wherein RX* is a reactive moiety that has reacted with a moiety on an antibody construct to form a conjugate.

In some embodiments, —X³ is represented by the formula:

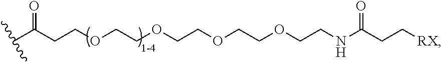

wherein RX is a reactive moiety, such as a maleimide.

In some embodiments, —X³ is represented by the formula:

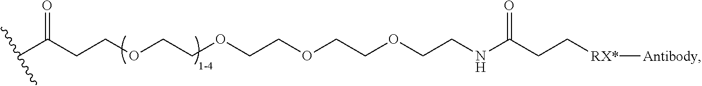

wherein RX* is a reactive moiety that has reacted with a moiety on an antibody construct to form a conjugate, such as an antibody drug conjugate.

In some embodiments, the compound is represented by the formula:

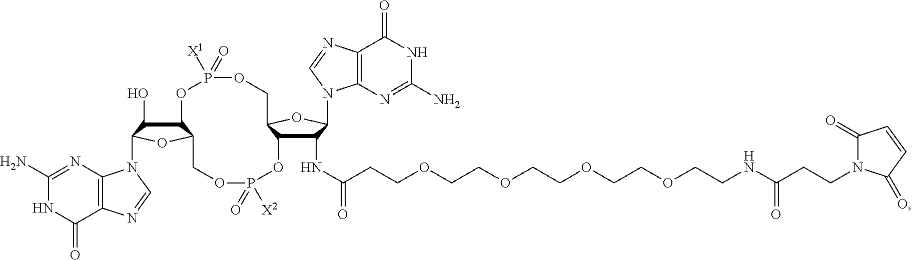

or a pharmaceutically acceptable salt thereof. The compound may be represented by the formula:

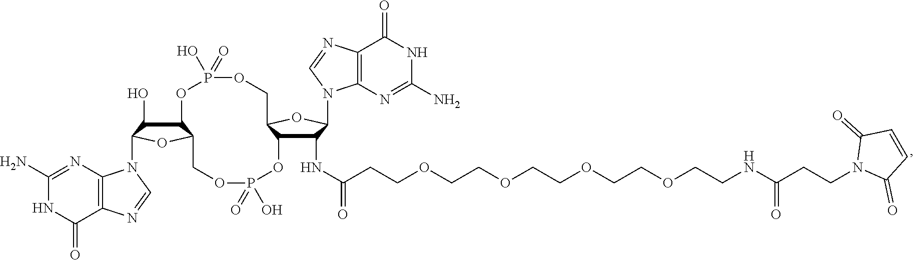

or a pharmaceutically acceptable salt thereof.

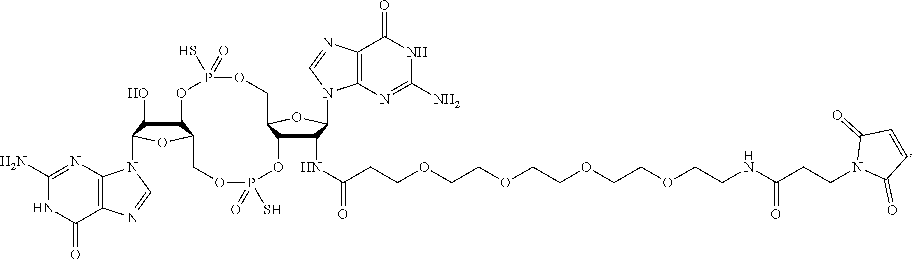

In some embodiments, the compound is represented by the formula:

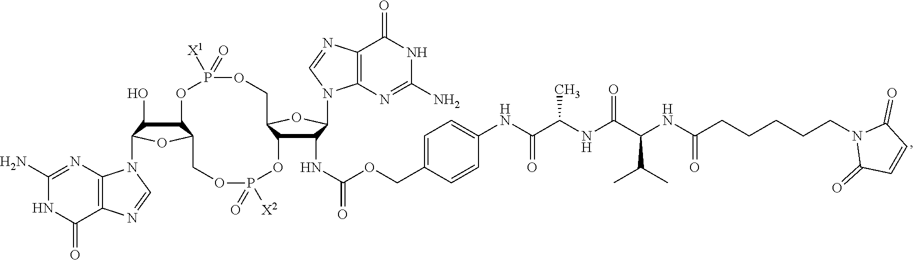

or a pharmaceutically acceptable salt thereof. The compound may be represented by the formula:

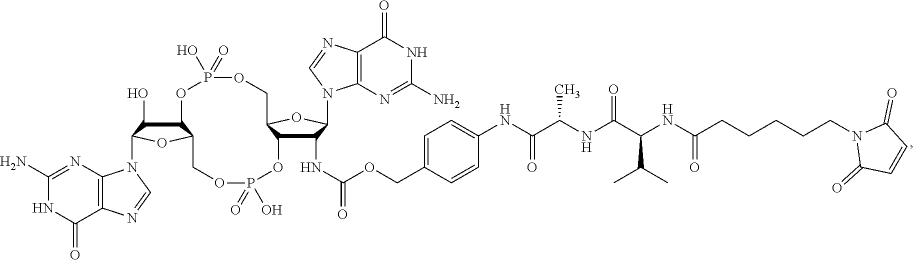

or a pharmaceutically acceptable salt thereof. The compound may be represented by the formula:

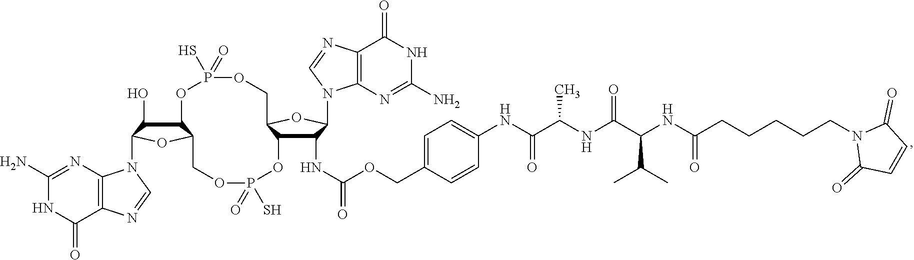

or a pharmaceutically acceptable salt thereof.

In some embodiments, the compound is represented by the formula:

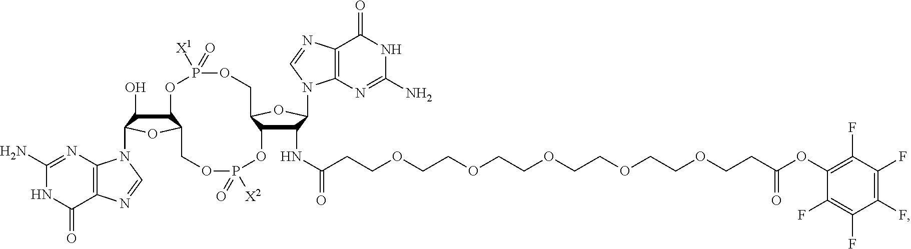

or a pharmaceutically acceptable salt thereof. The compound may be represented by the formula:

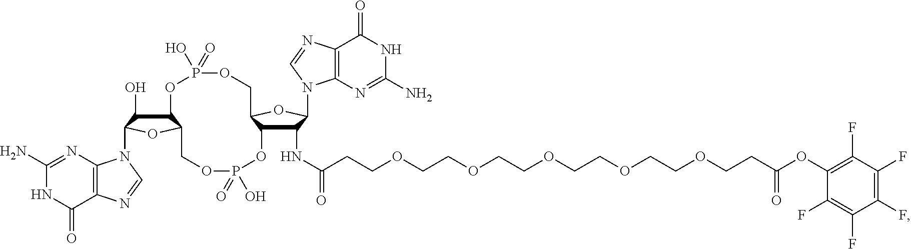

or a pharmaceutically acceptable salt thereof.

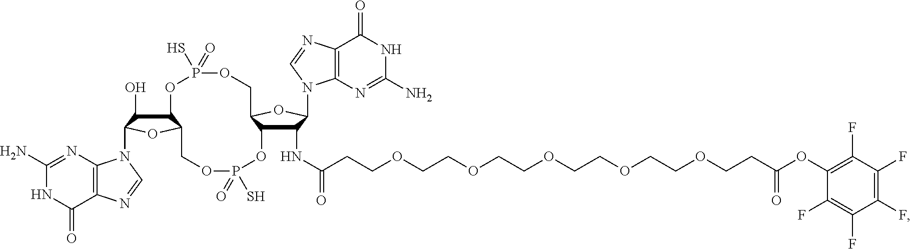

In some embodiments, the compound is represented by the formula:

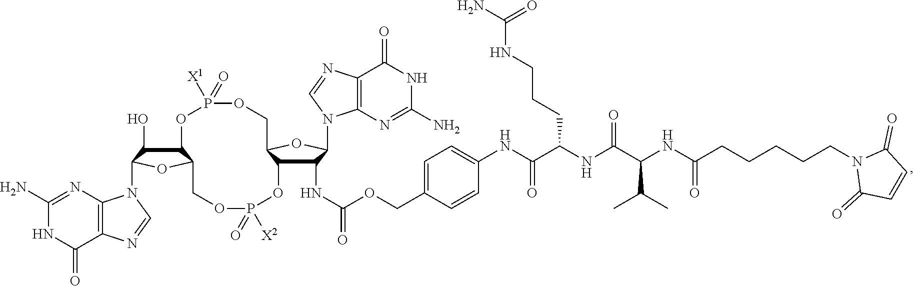

or a pharmaceutically acceptable salt thereof. The compound may be represented by the formula:

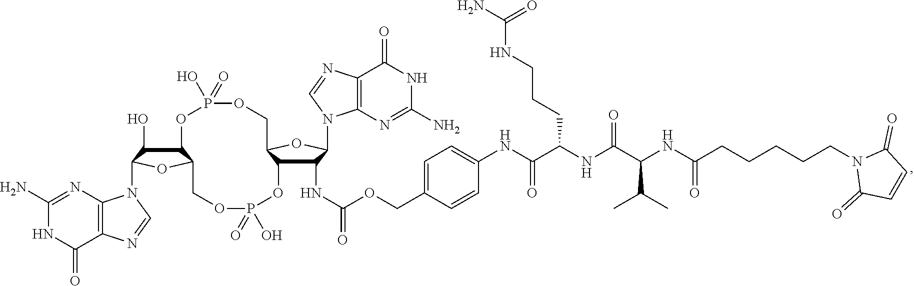

or a pharmaceutically acceptable salt thereof. The compound may be represented by the formula:

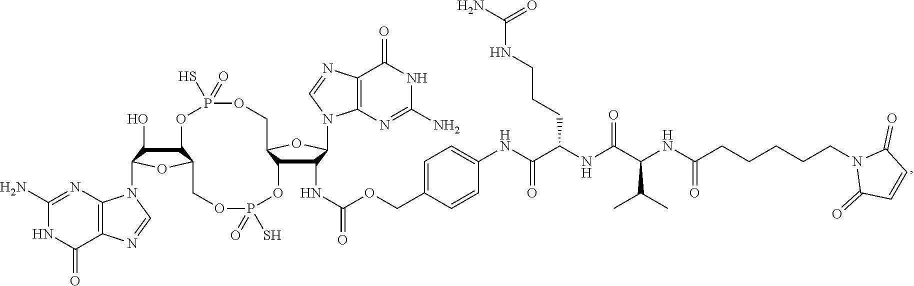

or a pharmaceutically acceptable salt thereof.

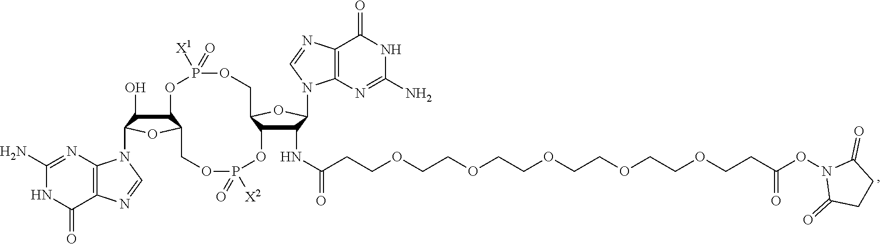

The compound is represented by the formula:

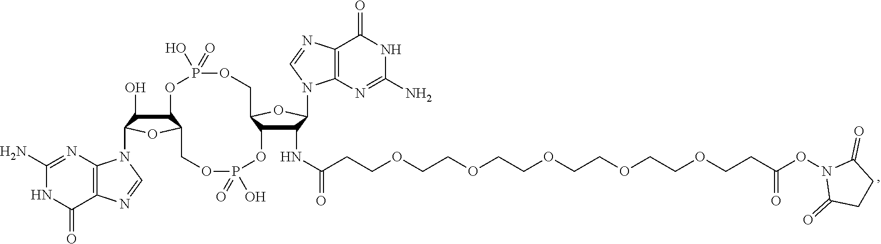

or a pharmaceutically acceptable salt thereof.

The compound may be represented by the formula:

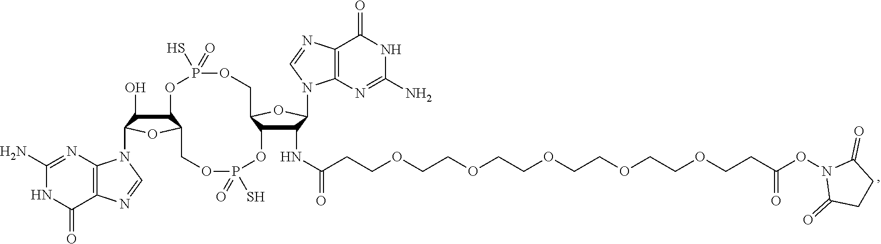

or a pharmaceutically acceptable salt thereof.

In some embodiments, the compound is represented by the formula:

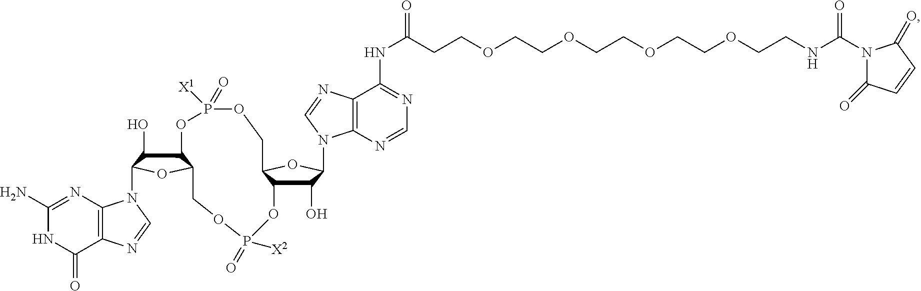

or a pharmaceutically acceptable salt thereof. The compound may be represented by the formula:

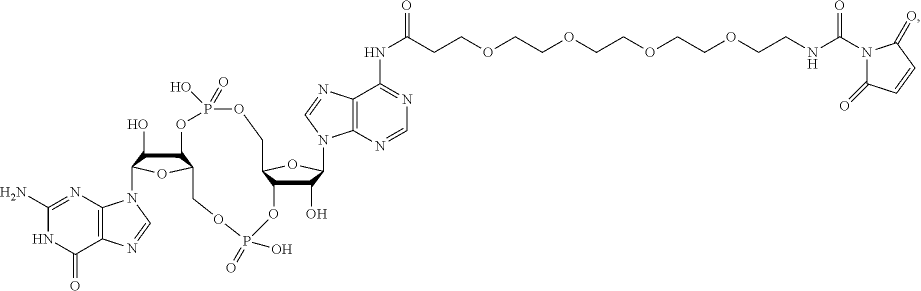

or a pharmaceutically acceptable salt thereof. The compound may be represented by the formula:

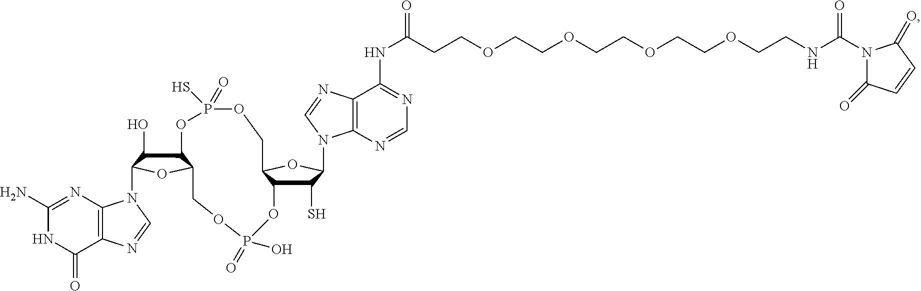

or a pharmaceutically acceptable salt thereof.

In some embodiments, the compound is represented by the formula:

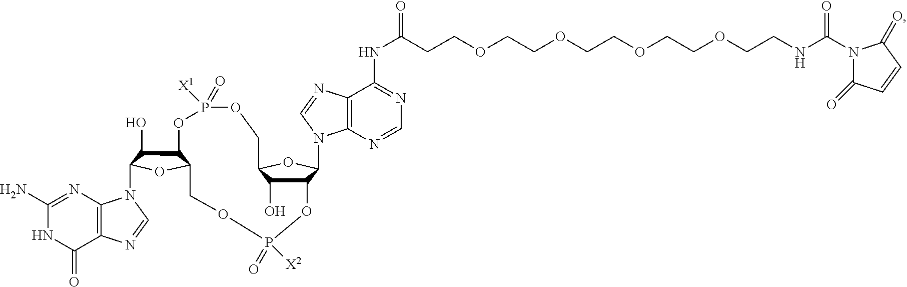

or a pharmaceutically acceptable salt thereof. The compound may be represented by the formula:

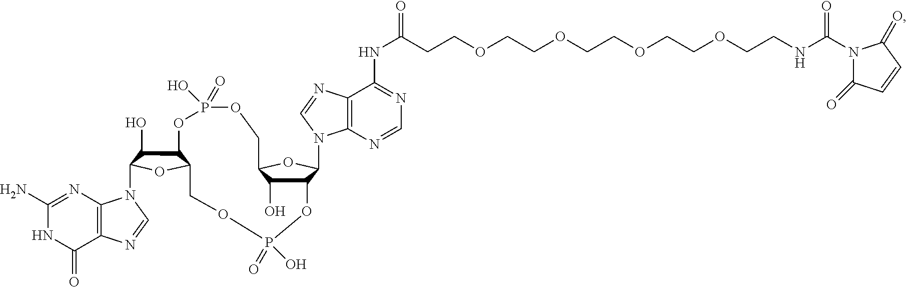

or a pharmaceutically acceptable salt thereof. The compound may be represented by the formula:

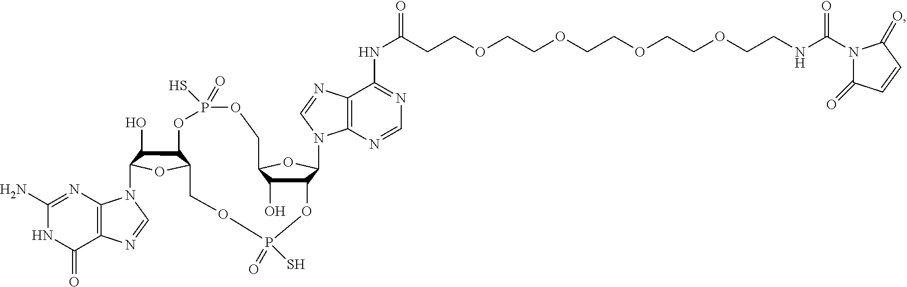

for a pharmaceutically acceptable salt thereof.

In some embodiments, the compound is represented by the formula:

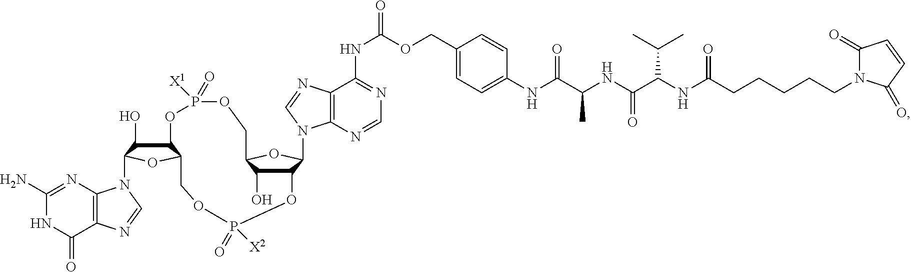

or a pharmaceutically acceptable salt thereof. The compound may be represented by the formula:

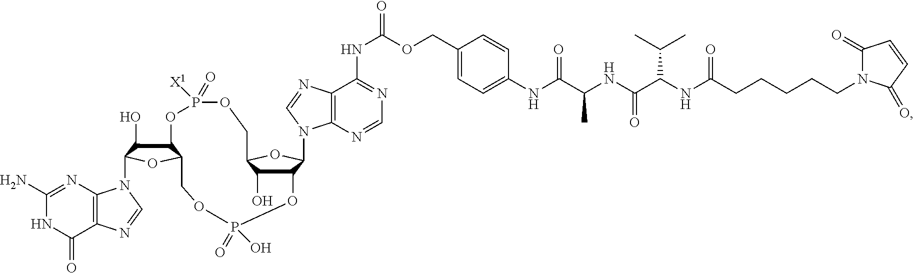

or a pharmaceutically acceptable salt thereof. The compound may be represented by the formula:

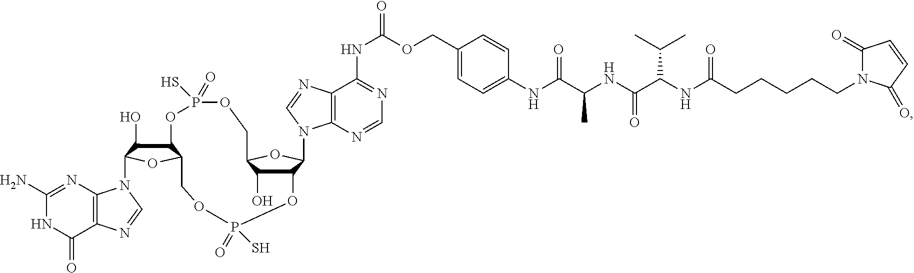

or a pharmaceutically acceptable salt thereof.

In some embodiments, the compound is represented by the formula:

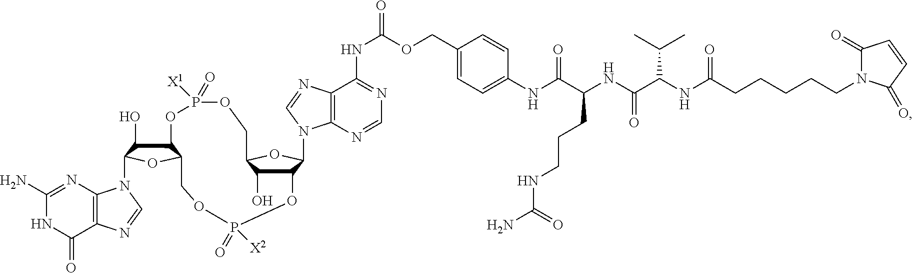

or a pharmaceutically acceptable salt thereof. The compound may be represented by the formula:

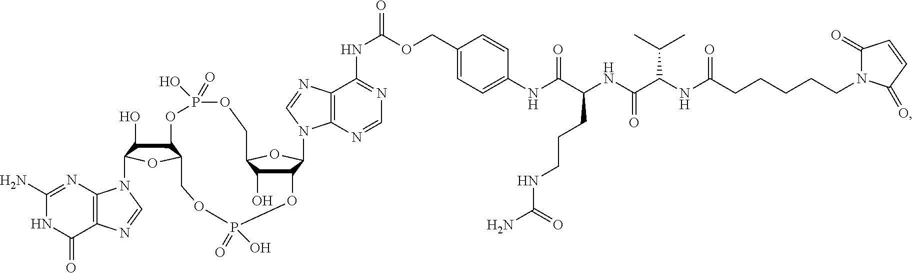

or a pharmaceutically acceptable salt thereof. The compound may be represented by the formula:

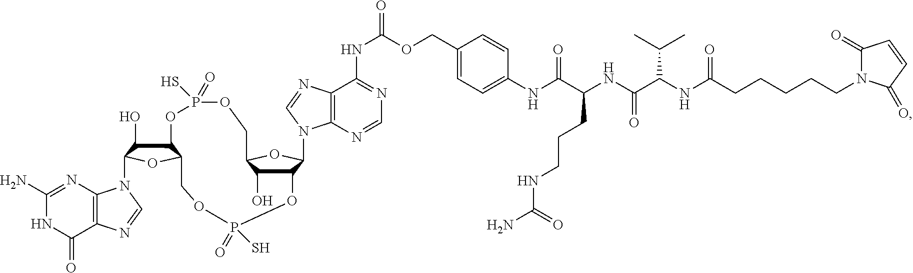

or a pharmaceutically acceptable salt thereof.

In some embodiments, the immune-stimulatory compound is a damage-associated molecular pattern molecule or a pathogen-associated molecular pattern molecule.

In some embodiments, the immune-stimulatory compound is a Toll-like receptor agonist, STING agonist, or RIG-I agonist.

In some embodiments, the immune-stimulatory compound is a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663.

In some embodiments, the immune-stimulatory compound is an inhibitor of TGFB, Beta-Catenin, PI3K-beta, STAT3, IL-10, IDO, or TDO. In some embodiments, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843.

In some embodiments, the immune-stimulatory compound does not reduce the affinity of the recombinant bispecific antibody for binding to the tumor associated antigen or to the antigen on the antigen presenting cell.

### Chemotherapeutic Agent Recombinant Bispecific Antibody Conjugates

In certain embodiments, the recombinant bispecific antibodies further comprise a chemotherapeutic compound. The recombinant bispecific antibody further comprising a chemotherapeutic compound can be a recombinant bispecific antibody conjugate. The chemotherapeutic compound can be coupled to the Fc region of the recombinant bispecific antibody. In certain embodiments, the chemotherapeutic compound is covalently coupled to the recombinant bispecific antibody by a linker creating a recombinant bispecific antibody conjugate. In certain embodiments, the coupled chemotherapeutic compound comprises an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), an inhibitor of topoisomerase (e.g., irinotecan, topotecan, amsacrine, etoposide, or teniposide), a kinase inhibitor (e.g., bortezomib, erlotinib, gefitinib, imatinib, vemurafenib, or vismodegib), a nucleoside analog or precursor analog (e.g., azacitidine, azathioprine, capecitabine, cytarabine, fluorouracil, gemcitabine, hydroxyurea, mercaptopurine, methotrexate, or thioguanine), a peptide antibiotic (e.g., actinomycin or bleomycin), a platinum-based agent (e.g., cisplatin, oxaloplatin, or carboplatin), or a plant alkaloid (e.g., vincristine, vinblastine, vinorelbine, vindesine, podophyllotoxin, paclitaxel, or docetaxel). In some embodiments, the chemotherapeutic agent is a nucleoside analog. In some embodiments, the chemotherapeutic agent is gemcitabine. The chemotherapeutic compound can be coupled to the recombinant bispecific antibody via a linker, as further described herein.

### Linkers

The conjugates and methods of using such conjugates described herein include conjugates that can comprise a linker, e.g., cleavable or non-cleavable linker, attached to an antibody construct or to a recombinant bispecific antibody. Linkers of the conjugates and methods described herein may not affect the binding of active portions of a conjugate (e.g., active portions include antigen binding domains, Fc domains, Fc comprising domains, binding domains, antibodies (e.g., recombinant bispecific antibodies), antibody constructs, agonists or the like) to a target, which can be a cognate binding partner such as an antigen. A linker can form a linkage between different parts of a conjugate. A conjugate can comprise multiple linkers. These linkers can be the same linkers or different linkers. As will be appreciated by the skilled artisan, the following description of conjugates comprising antibody constructs is applicable to conjugates comprising recombinant bispecific antibodies.

As will be appreciated by skilled artisans, the linkers can link the immune-stimulatory compound to the antibody construct of the conjugate by forming a covalent linkage to the immune-stimulatory compound at one location and a covalent linkage to the antibody construct of the conjugate at another location. The covalent linkages can be formed by reaction between functional groups on the linker and functional groups on the compounds and antibody construct. As used herein, the expression “linker” can include (i) unconjugated forms of the linker that can include a functional group capable of covalently linking the linker to an immune-stimulatory compound and a functional group capable of covalently linking the linker to an antibody construct; (ii) partially conjugated forms of the linker that can include a functional group capable of covalently linking the linker to an antibody construct of the conjugate and that can be covalently linked to an immune-stimulatory compound, or vice versa; and (iii) fully conjugated forms of the linker that can be covalently linked to both an immune-stimulatory compound and an antibody construct. In some specific embodiments immune-stimulatory conjugates described herein, moieties comprising the functional groups on the linker and covalent linkages formed between the linker and antibody construct of the conjugate can be specifically illustrated as Rx and LK, respectively. One embodiment pertains to a conjugate formed by contacting an antibody construct that binds to a cell surface receptor or tumor antigen expressed on a tumor cell with a linker described herein under conditions in which the linker covalently links to the antibody construct. One embodiment pertains to a method of making a conjugate formed by contacting a linker described herein under conditions in which the linker covalently links to an antibody construct. One embodiment pertains to a method of stimulating immune activity in a cell that expresses CD40, comprising contacting the cell with a conjugate described herein that is capable of binding the cell, under conditions in which the conjugate binds the cell.

Attachment via a linker can involve incorporation of a linker between parts of a conjugate. A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker can be contain a valine-citrulline peptide or a valine-alanine peptide. A valine-citrulline- or valine-alanine-containing linker can contain a pentafluorophenyl group. A valine-citrulline or valine-alanine-containing linker can contain a succimide or a maleimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a succinimide group. A valine-citrulline- or valine-alanine-containing linker can contain a PABA group and a maleimide group. A non-cleavable linker can be protease insensitive. A non-cleavable linker can contain a maleimide group. A non-cleavable linker can contain a succinimide group. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can be a THIOMAB linker. A THIOMAB linker can be a (maleimidocaproyl)-(valine-citrulline)-(para-aminobenzyloxycarbonyl) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 672) to an N-terminal GGG motif to regenerate a native amide bond. The linker created can therefore link a moiety attached to the LXPTG recognition motif (SEQ ID NO: 672) with a moiety attached to the N-terminal GGG motif. A linker can be a link created between an unnatural amino acid on one moiety reacting with oxime bond that was formed by modifying a ketone group with an alkoxyamine on another moiety. A moiety can be a conjugate. A moiety can be an antibody construct, such as an antibody. A moiety can be an immune-stimulatory compound. A moiety can be a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In a conjugate as described herein, the immune-stimulatory compound can be linked to the antibody construct of the conjugate by way of linkers. The linker linking an immune-stimulatory compound to the antibody construct of the conjugate can be short, long, hydrophobic, hydrophilic, flexible or rigid, or may be composed of segments that each independently have one or more of the above-mentioned properties such that the linker may include segments having different properties. The linkers can be polyvalent such that they covalently link more than one immune-stimulatory compound to a single site on the antibody construct, or monovalent such that covalently they link a single immune-stimulatory compound to a single site on the antibody construct of the the conjugate.

Exemplary polyvalent linkers that may be used to link many immune-stimulatory compounds to an antibody construct of the conjugate are described. For example, Fleximer® linker technology has the potential to enable high-DAR conjugate with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with immune-stimulatory compound as shown in the Scheme below.

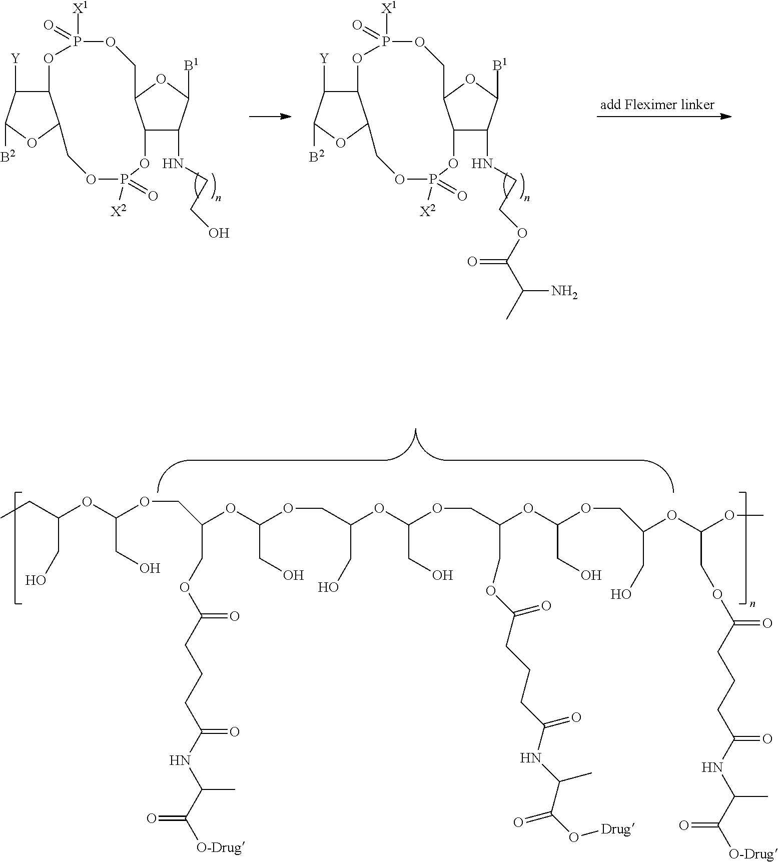

To utilize the Fleximer® linker technology depicted in the scheme above, an aliphatic alcohol can be present or introduced into the immune-stimulatory compound. The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an immune-stimulatory compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate immune-stimulatory compound release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the immune-stimulatory compound once the conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

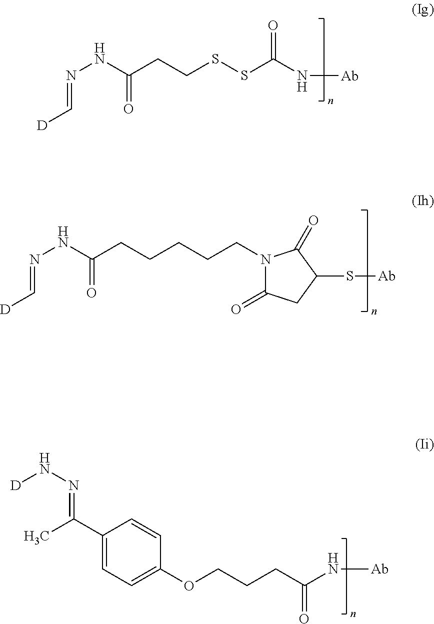

wherein D and Ab represent the immune-stimulatory compound and antibody construct, respectively, and n represents the number of immune-stimulatory compound—linkers linked to the antibody construct. In certain linkers such as linker (Ig), the linker can comprise two cleavable groups—a disulfide and a hydrazone moiety. For such linkers, effective release of the unmodified free immune-stimulatory compound can require acidic pH or disulfide reduction and acidic pH. Linkers such as (Ih) and (Ii) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the immune-stimulatory compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the immune-stimulatory compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Conjugates including exemplary disulfide-containing linkers can include the following structures:

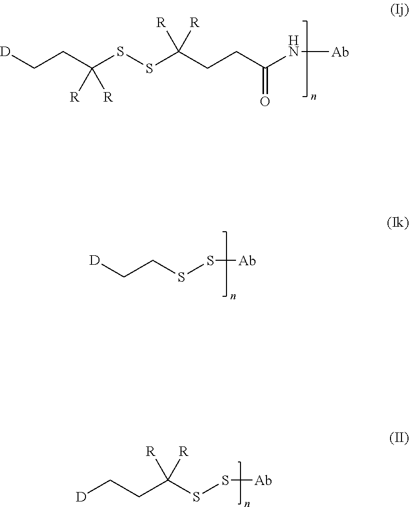

wherein D and Ab represent the immune-stimulatory compound and antibody construct, respectively, n represents the number of immune-stimulatory compound-linkers linked to the antibody construct and R is independently selected at each occurrence from hydrogen or alkyl, for example. Increasing steric hindrance adjacent to the disulfide bond can increase the stability of the linker. Structures such as (Ij) and (Il) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of an immune-stimulatory compound from a conjugate can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

In a linker, a cleavable peptide can be selected from tetrapeptides such as Gly-Phe-Leu-Gly (SEQ ID NO: 1332), Ala-Leu-Ala-Leu (SEQ ID NO: 1333) or dipeptides such as Val-Cit, Val-Ala, and Phe-Lys. Dipeptides can have lower hydrophobicity compared to longer peptides, depending on the composition of the peptide.

A variety of dipeptide-based cleavable linkers can be used in the conjugates described herein.

Enzymatically cleavable linkers can include a self-immolative spacer to spatially separate the immune-stimulatory compound from the site of enzymatic cleavage. The direct attachment of an immune-stimulatory compound to a peptide linker can result in proteolytic release of an amino acid adduct of the immune-stimulatory compound, thereby impairing its activity. The use of a self-immolative spacer can allow for the elimination of the fully active, chemically unmodified immune-stimulatory compound upon amide bond hydrolysis.

One self-immolative spacer can be a bifunctional para-aminobenzyl alcohol group, which can link to the peptide through the amino group, forming an amide bond, while amine containing immune-stimulatory compounds can be attached through carbamate functionalities to the benzylic hydroxyl group of the linker (to give a p-amidobenzylcarbamate, PABC). The resulting pro-immune-stimulatory compound can be activated upon protease-mediated cleavage, leading to a 1,6-elimination reaction releasing the unmodified immune-stimulatory compound, carbon dioxide, and remnants of the linker group. The following scheme depicts the fragmentation of p-amidobenzyl carbamate and release of the immune-stimulatory compound:

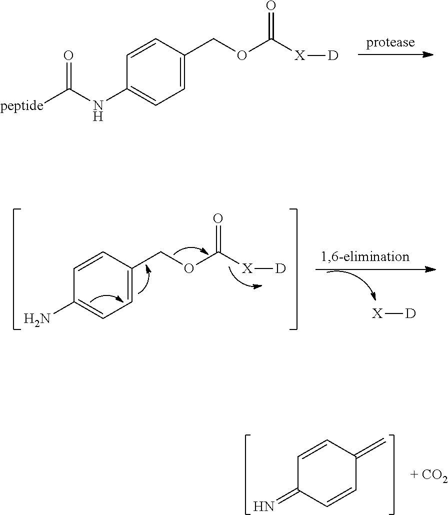

wherein X-D represents the unmodified immune-stimulatory compound.

The enzymatically cleavable linker can be a ß-glucuronic acid-based linker. Facile release of the immune-stimulatory compound can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of a conjugate to undergo aggregation due to the hydrophilic nature of ß-glucuronides. In certain embodiments, ß-glucuronic acid-based linkers can link an antibody construct to a hydrophobic immune-stimulatory compound. The following scheme depicts the release of an immune-stimulatory compound (D) from an antibody construct of the conjugate (Ab) containing a ß-glucuronic acid-based linker:

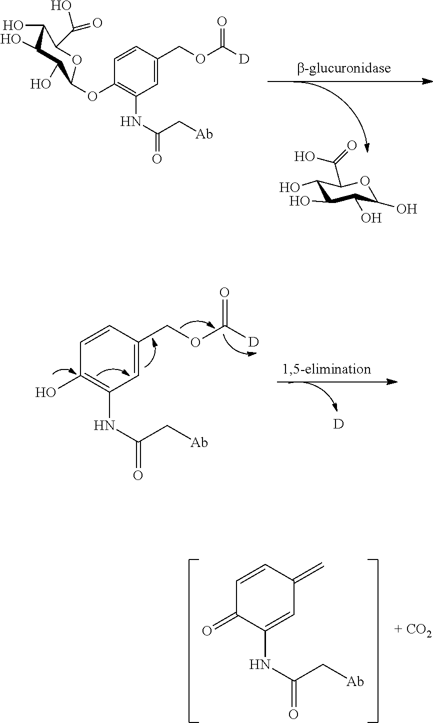

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the conjugates comprising an immune-stimulatory compound described herein. In certain embodiments, the enzymatically cleavable linker is a β-galactoside-based linker. β-Galactoside is present abundantly within lysosomes, while the enzyme activity outside cells is low.

Additionally, immune-stimulatory compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Immune-stimulatory compounds containing an aromatic or aliphatic hydroxyl group can be covalently bonded to a linker through the hydroxyl group using a methodology that relies on a methylene carbamate linkage, as described in WO 2015/095755.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-stimulatory compound, wherein such ester groups can hydrolyze under physiological conditions to release the immune-stimulatory compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can comprise an enzymatically cleavable peptide moiety, for example, a linker comprising structural formula (IVa), (IVb), (IVc), or (IVd):

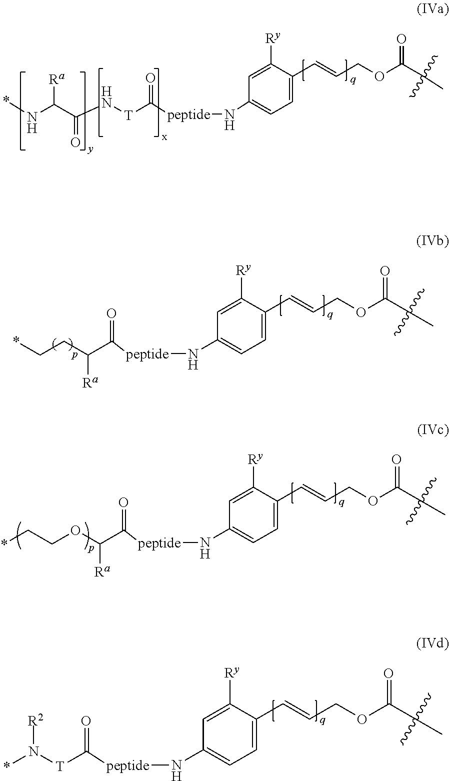

or a salt thereof, wherein: peptide represents a peptide (illustrated N→C, wherein peptide includes the amino and carboxy “termini”) a cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is [symbol] 0 or 1; [symbol] represents the point of attachment of the linker to the immune-stimulatory compound; and * represents the point of attachment to the remainder of the linker.

In certain embodiments, the peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) that can be included in the conjugates described herein can include the linkers illustrated below (as illustrated, the linkers include a group suitable for covalently linking the linker in a conjugate and the wavy line or unlinked bond indicates an attachment site for an immune-stimulatory compound):

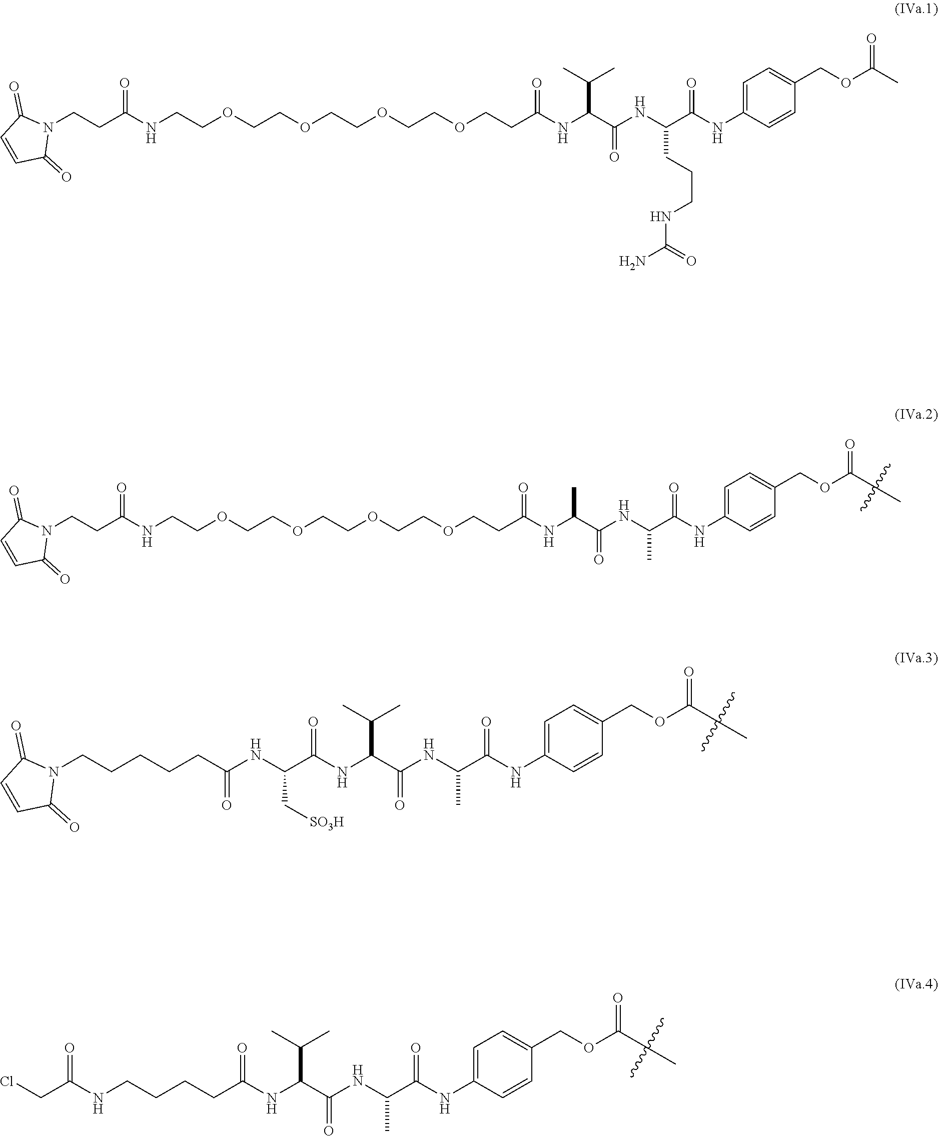 [SMILES: CC(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1]

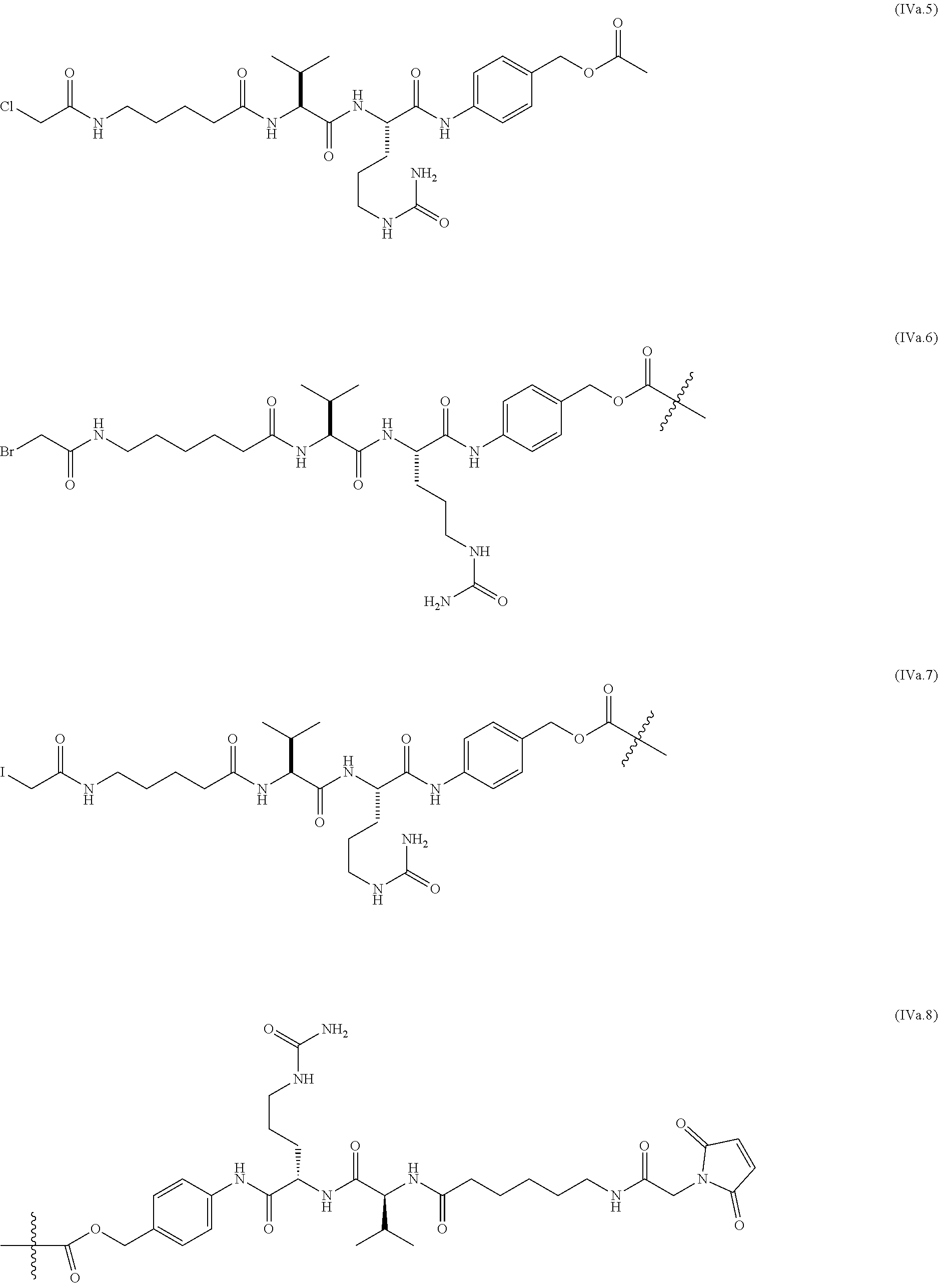 [SMILES: CC(=O)OCc1ccc(NC(=O)[C@H](CCCNC(N)=O)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

Exemplary embodiments of linkers according to structural formula (IVb), (IVc), or (IVd) that can be included in the conjugates described herein can include the linkers illustrated below (as illustrated, the linkers can include a group suitable for covalently linking the linker to a conjugate and the wavy line indicates an attachment site for an immune-stimulatory compound):

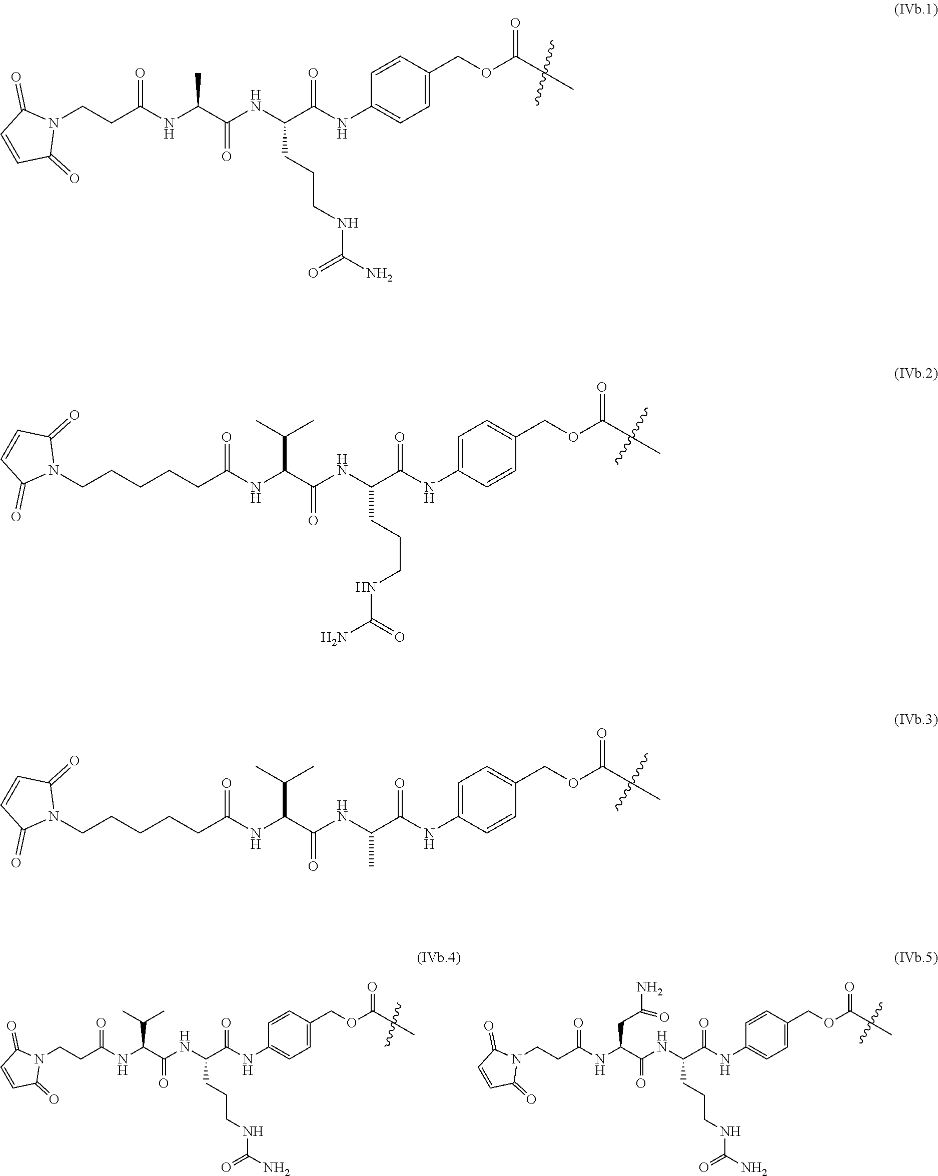 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

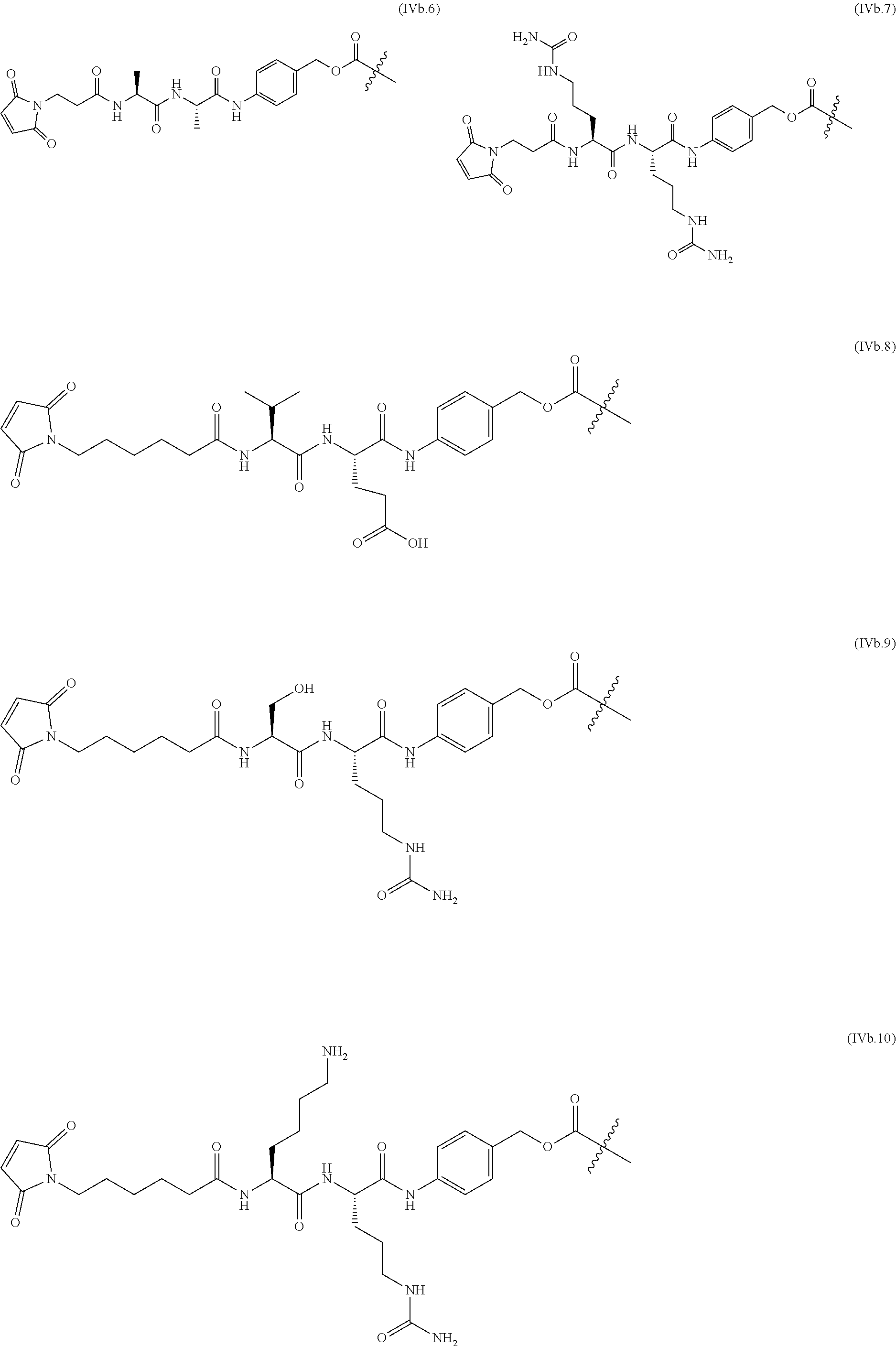 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

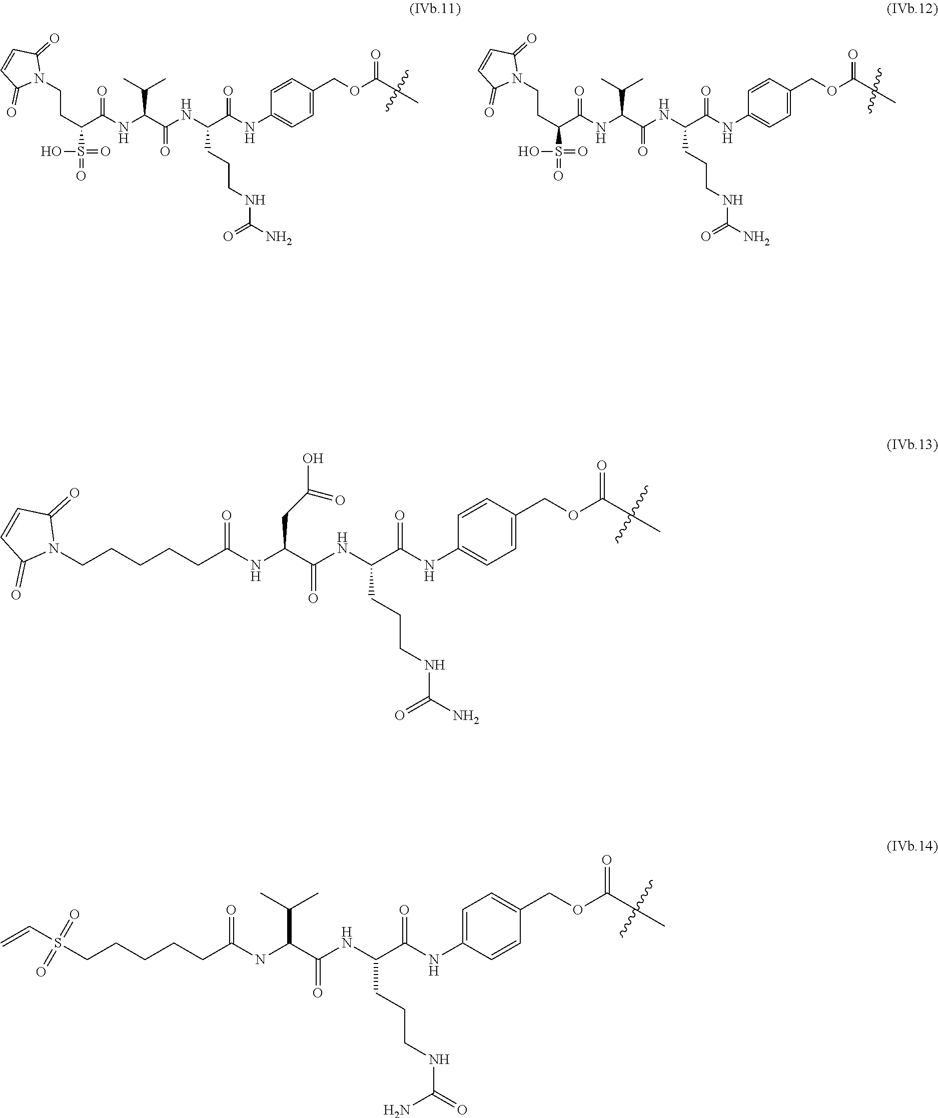 [SMILES: *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1]

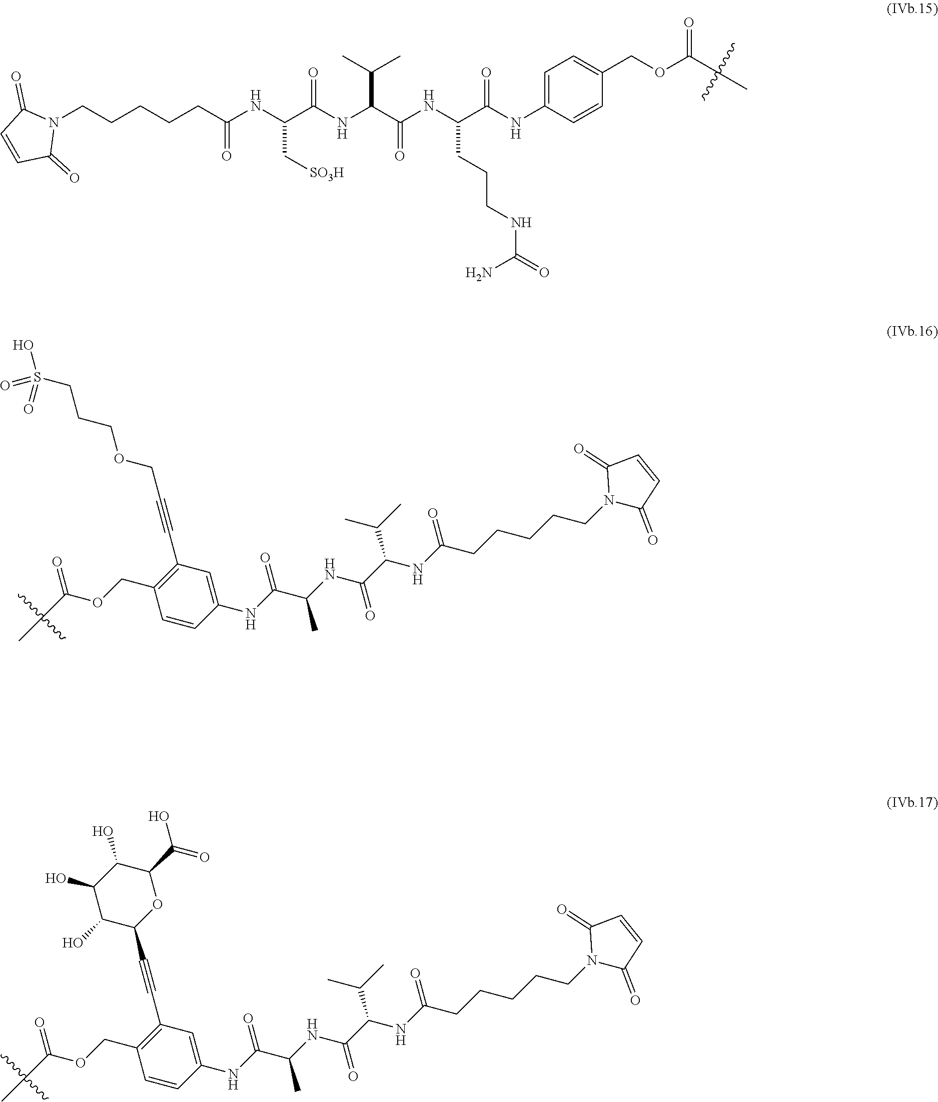 [SMILES: *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O]

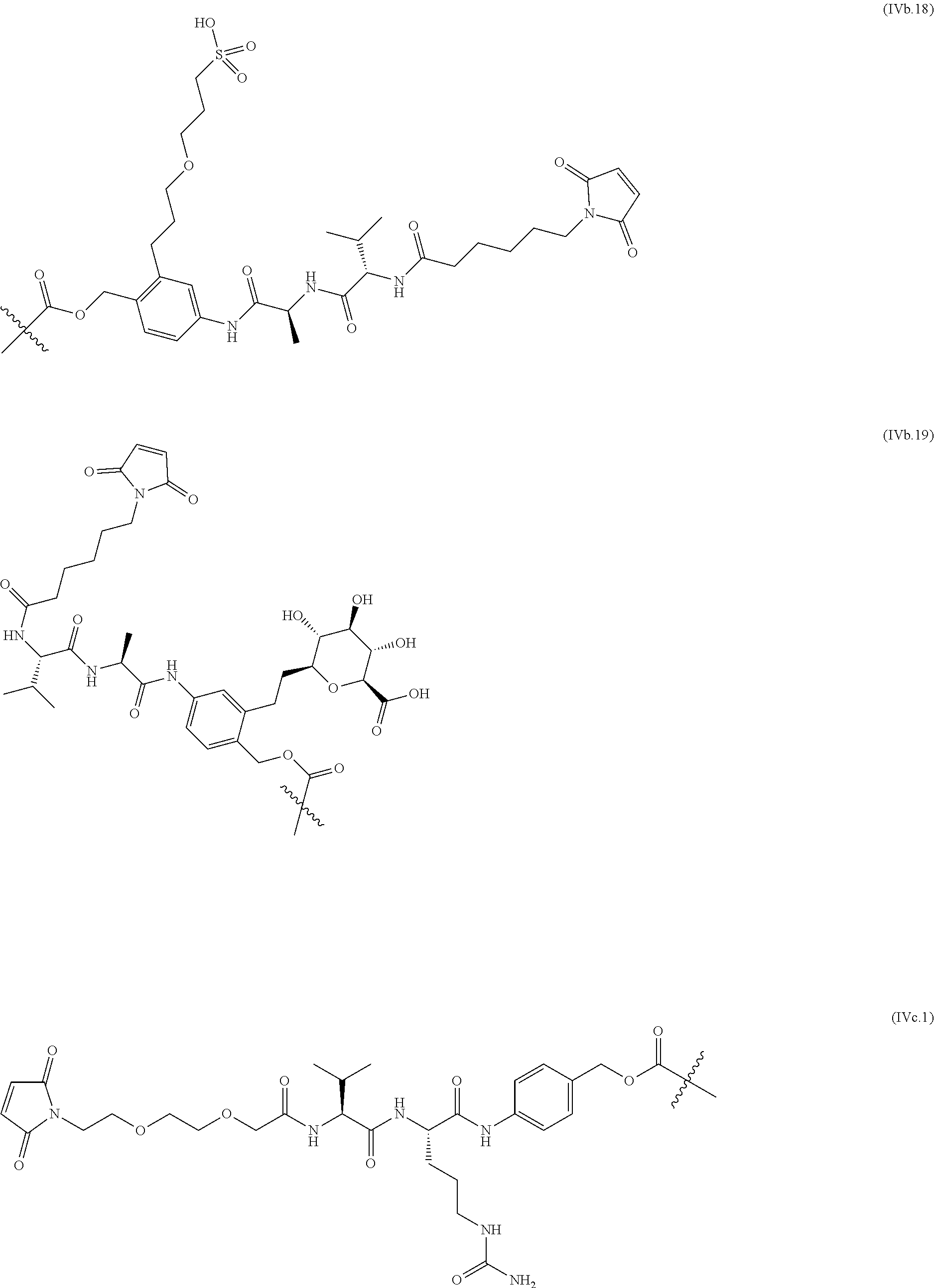 [SMILES: *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1]

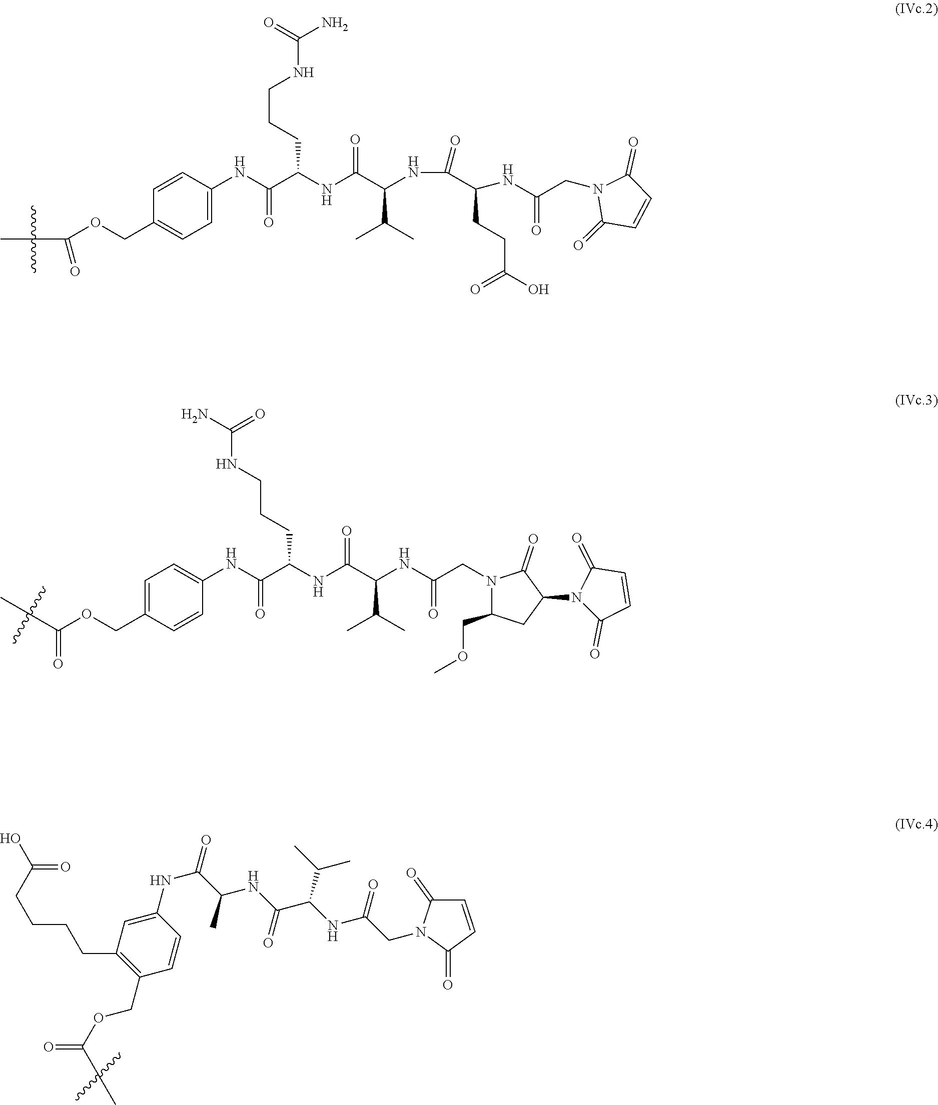 [SMILES: *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O]

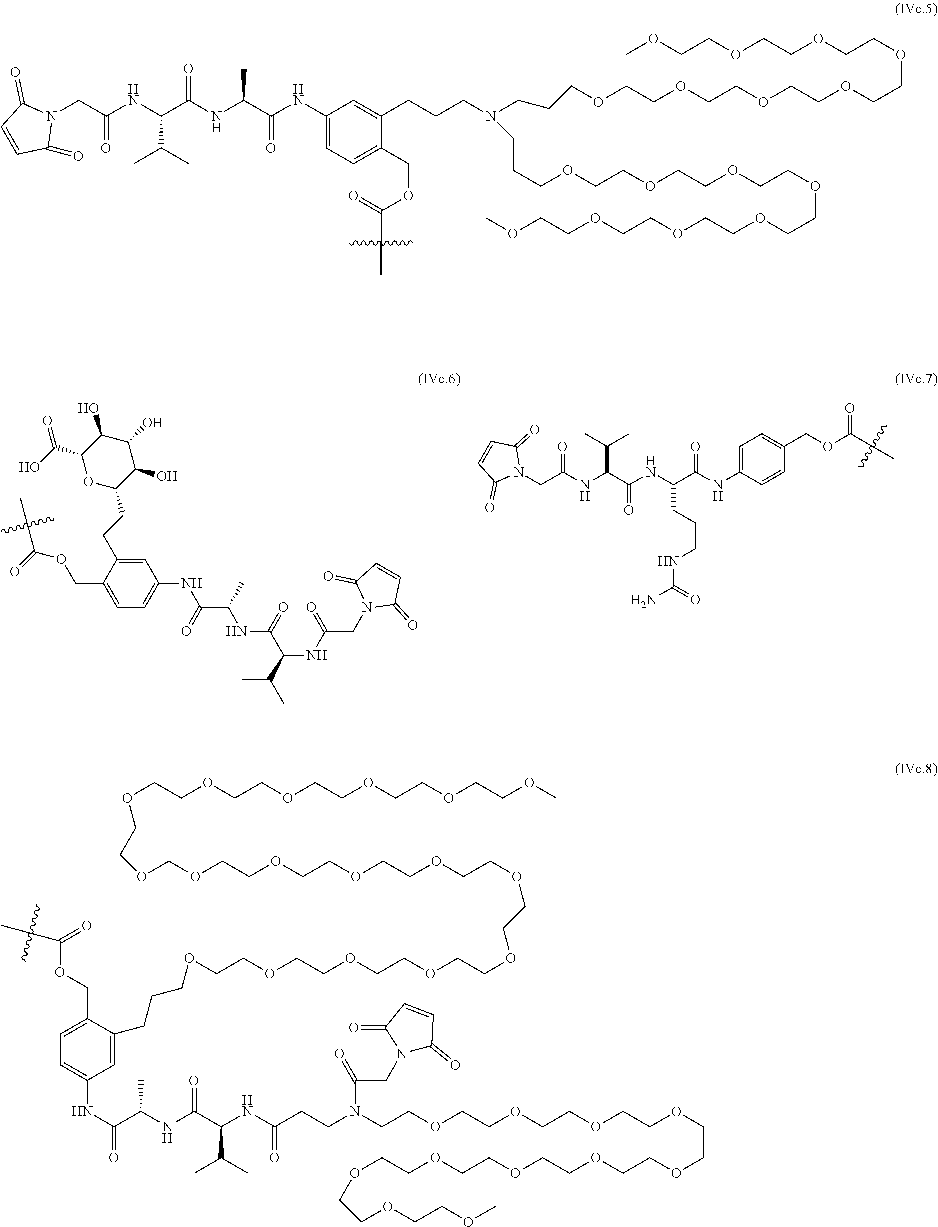

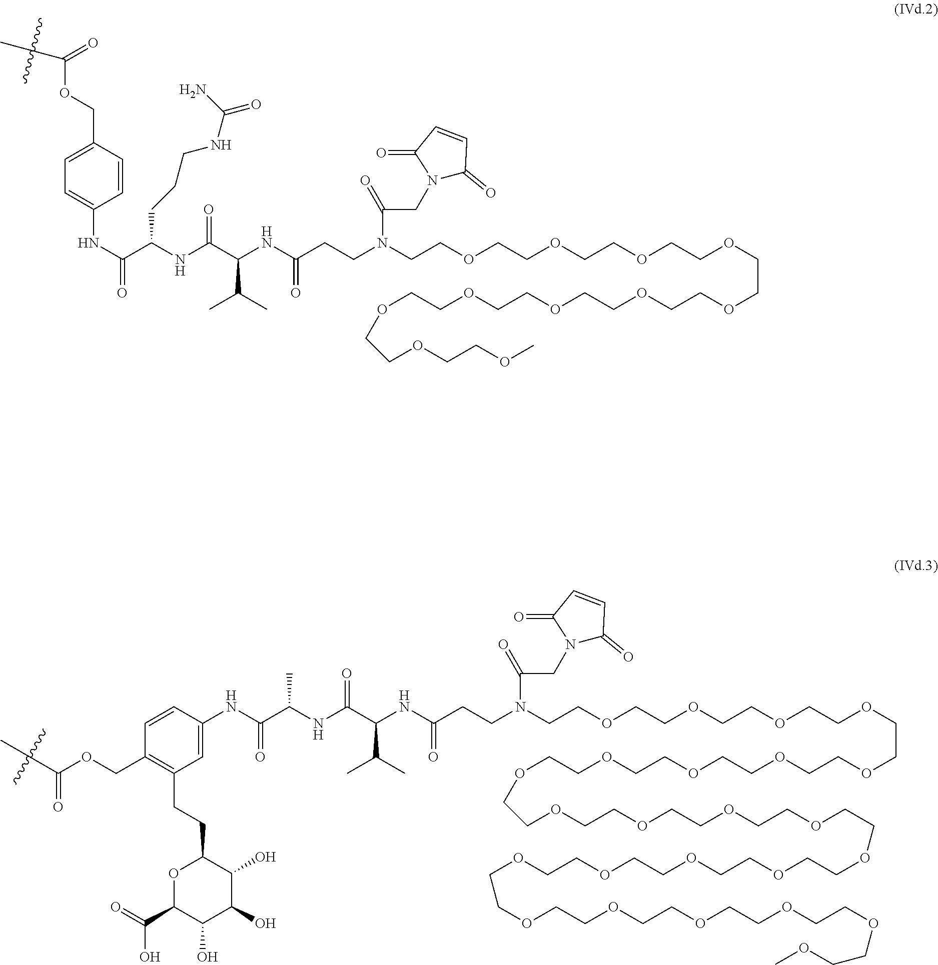 [SMILES: *C(=O)OCc1ccc(NC(=O)[C@H](CCCNC(N)=O)NC(=O)[C@@H](NC(=O)CCN(CCOCCOCCOCCOCCOCCOCCOCCOCCOCCOCCOC)C(=O)CN2C(=O)C=CC2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOCCOCCOCCOCCOCCOCCOCCOCCOCCOCCOCCOCCOCCOC)C(=O)CN2C(=O)C=CC2=O)C(C)C)cc1CC[C@@H]1O[C@H](C(=O)O)[C@@H](O)[C@H](O)[C@H]1O]

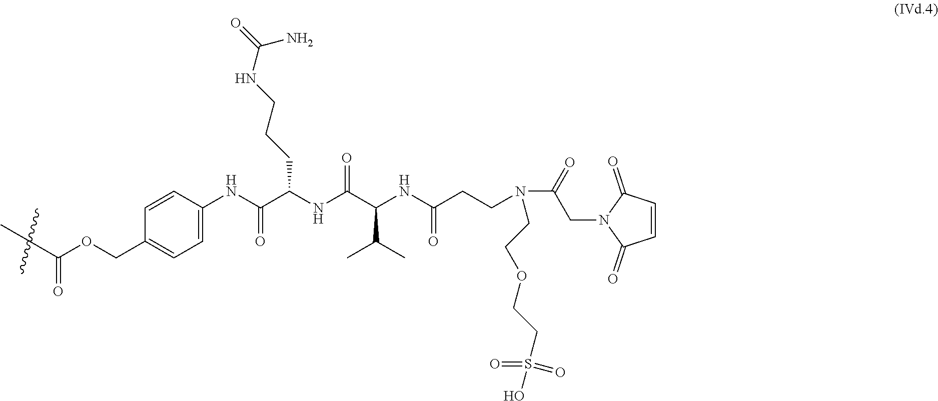 [SMILES: *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

The linker can contain an enzymatically cleavable sugar moiety, for example, a linker comprising structural formula (Va), (Vb), (Vc), (Vd), or (Ve):

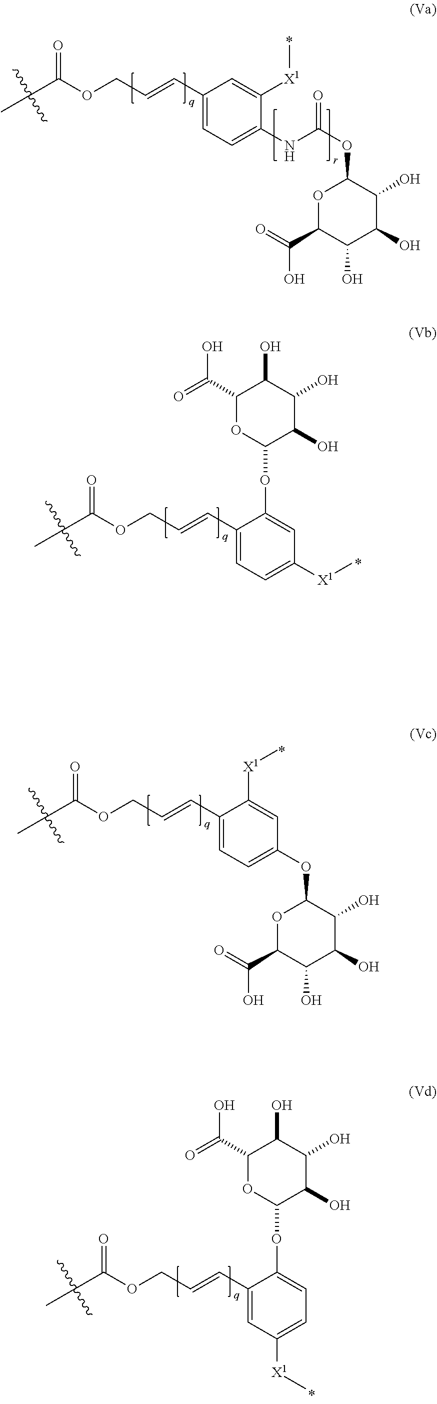

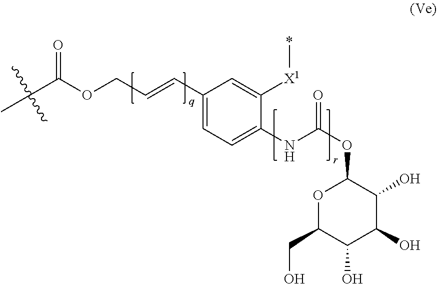

or a salt thereof, wherein: q is 0 or 1; r is 0 or 1; X¹ is CH₂, O or NH; [symbol] represents the point of attachment of the linker to the immune-stimulatory compound; and represents the point of attachment to the remainder of the linker.

Exemplary embodiments of linkers according to structural formula (Va) that may be included in the immune-stimulatory conjugates described herein can include the linkers illustrated below (as illustrated, the linkers include a group suitable for covalently linking the linker in a conjugate and the wavy line indicates an attachment site for an immune-stimulatory compound):

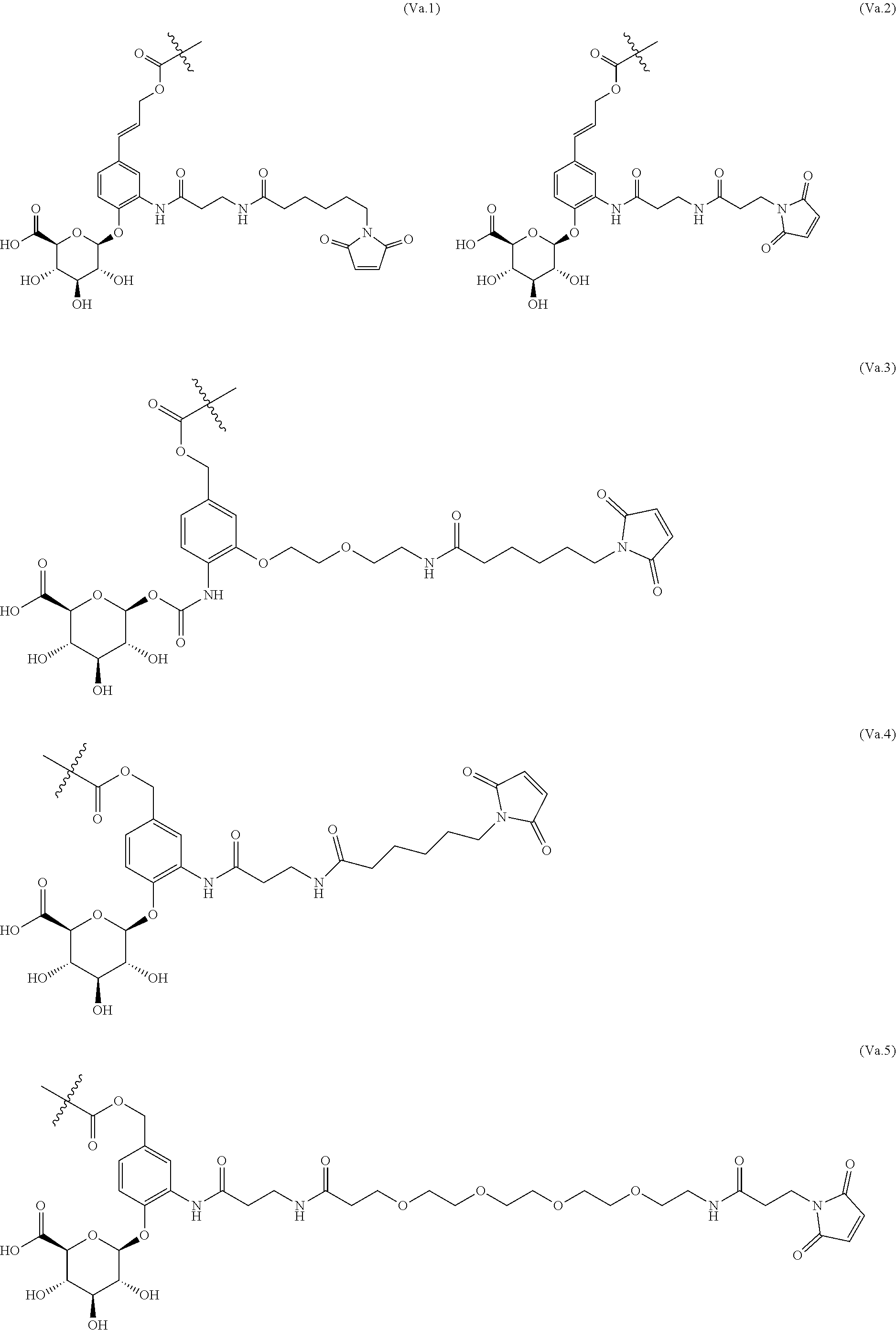

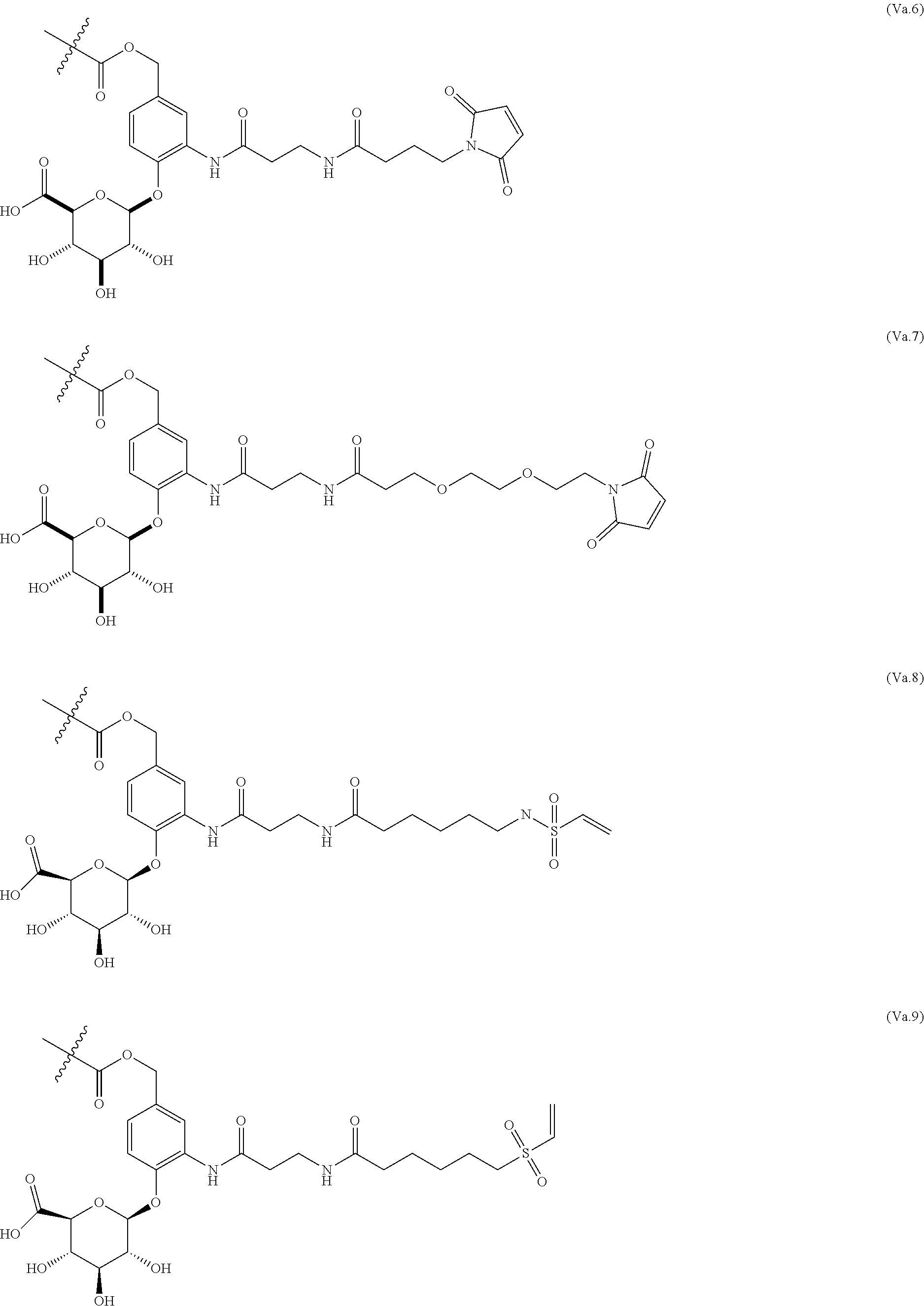

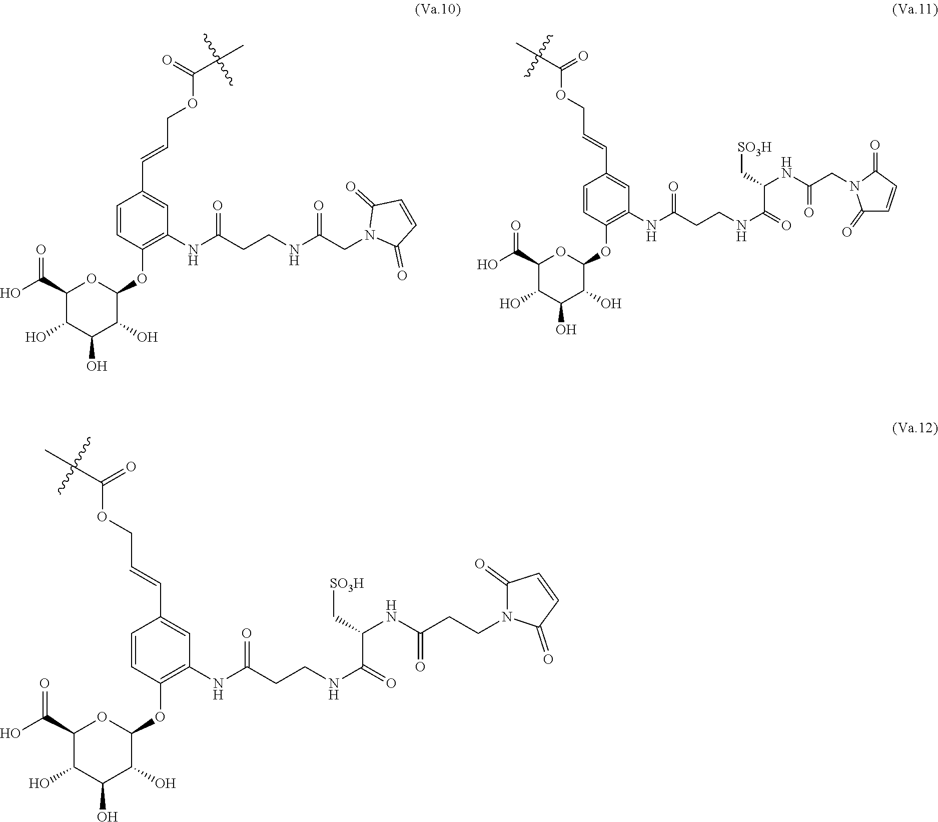 [SMILES: *C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of linkers according to structural formula (Vb) that may be included in the conjugates described herein include the linkers illustrated below (as illustrated, the linkers include a group suitable for covalently linking the linker in a conjugate and the wavy line indicates an attachment site for an immune-stimulatory compound):

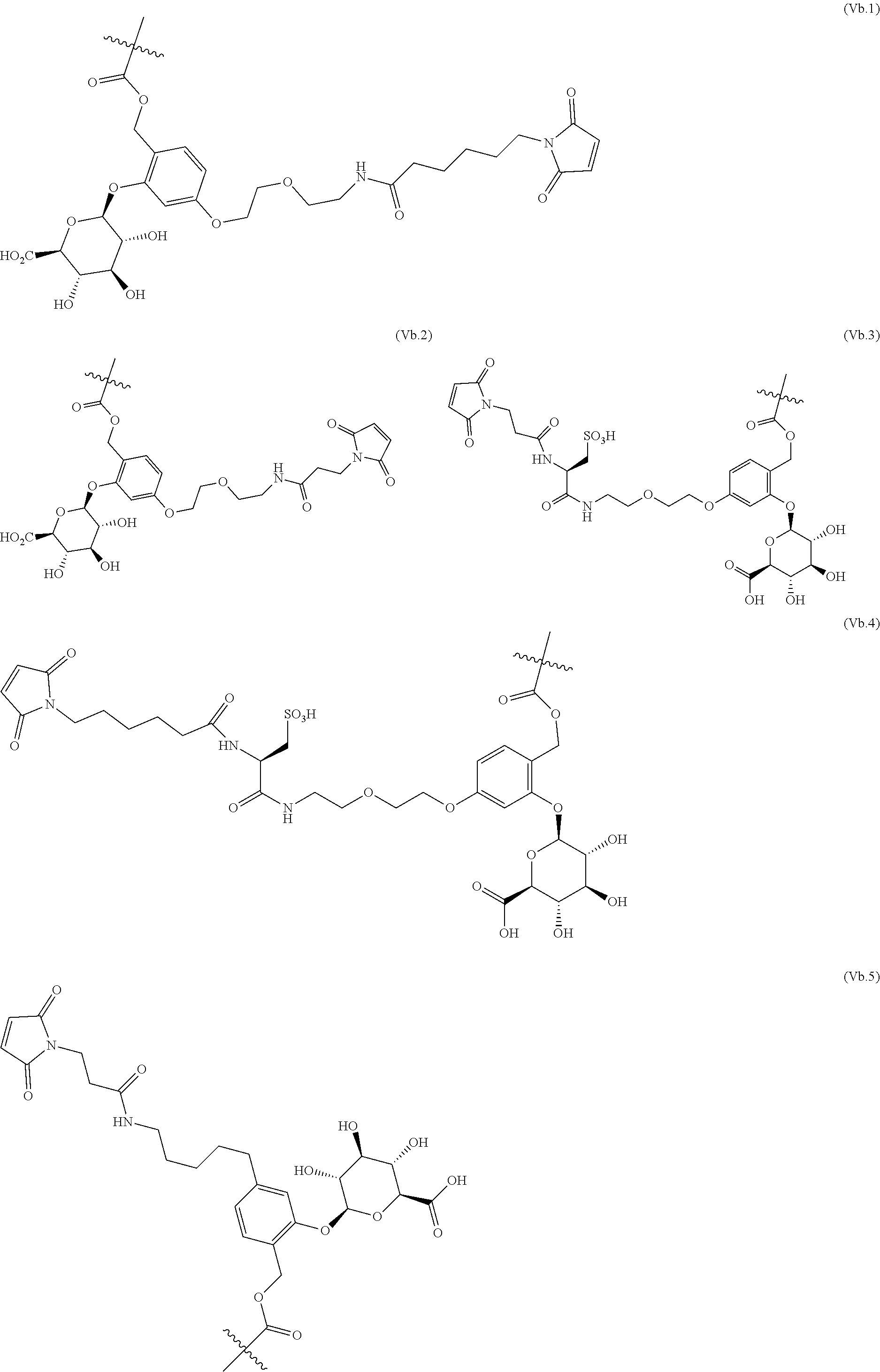

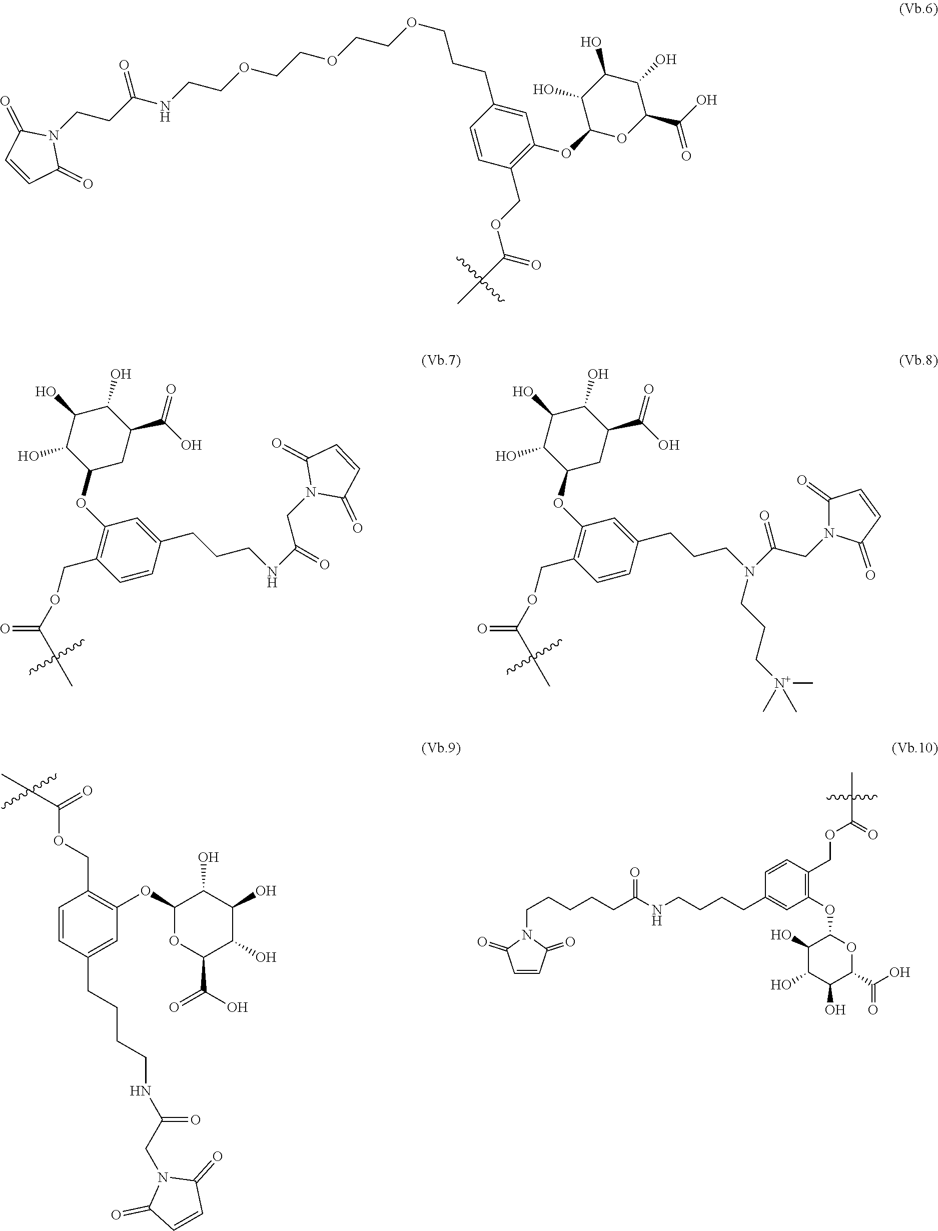

Exemplary embodiments of linkers according to structural formula (Vc) that may be included in the conjugates described herein include the linkers illustrated below (as illustrated, the linkers include a group suitable for covalently linking the linker in a conjugate and the wavy line indicates an attachment site for an immune-stimulatory compound):

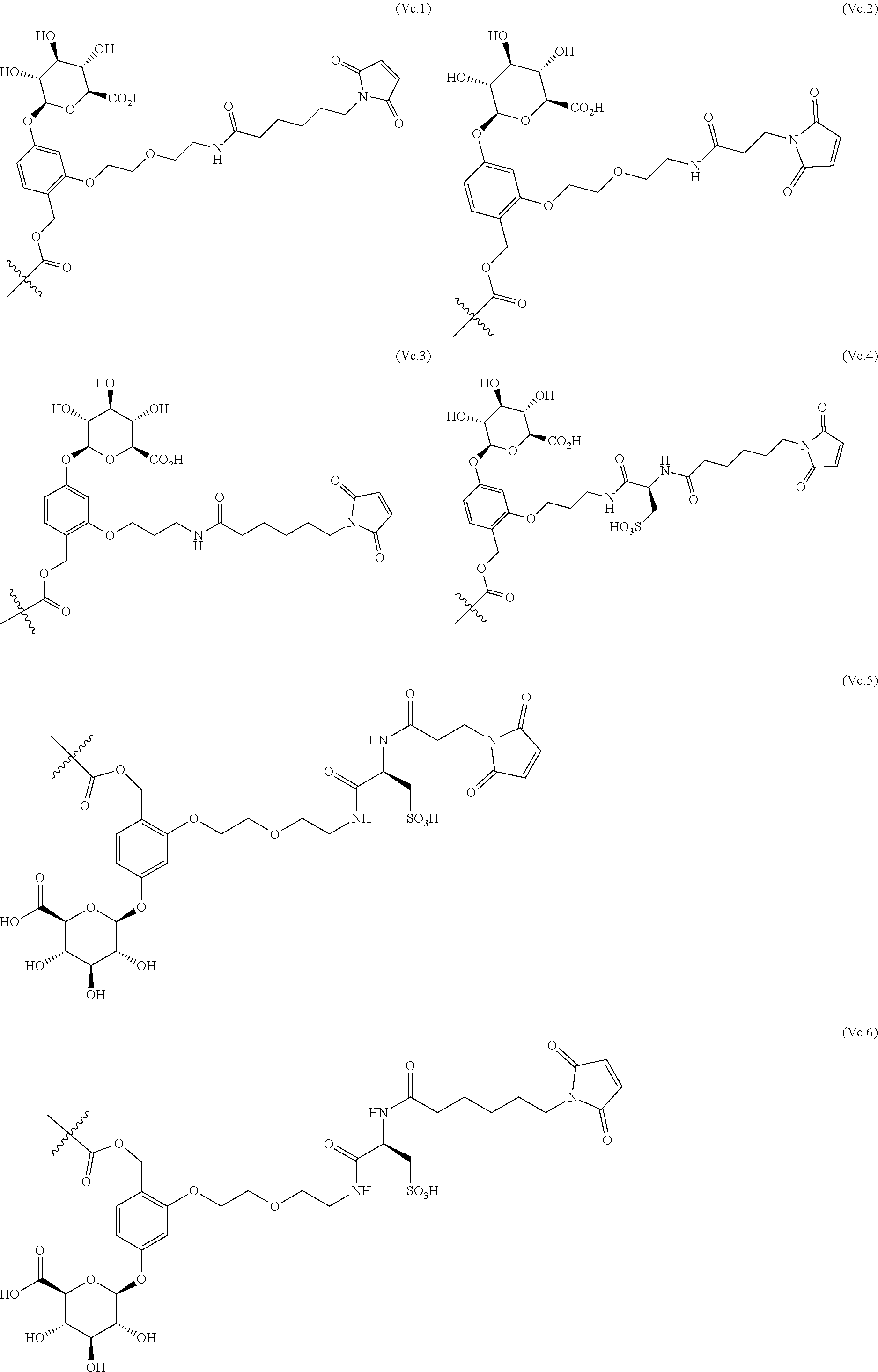

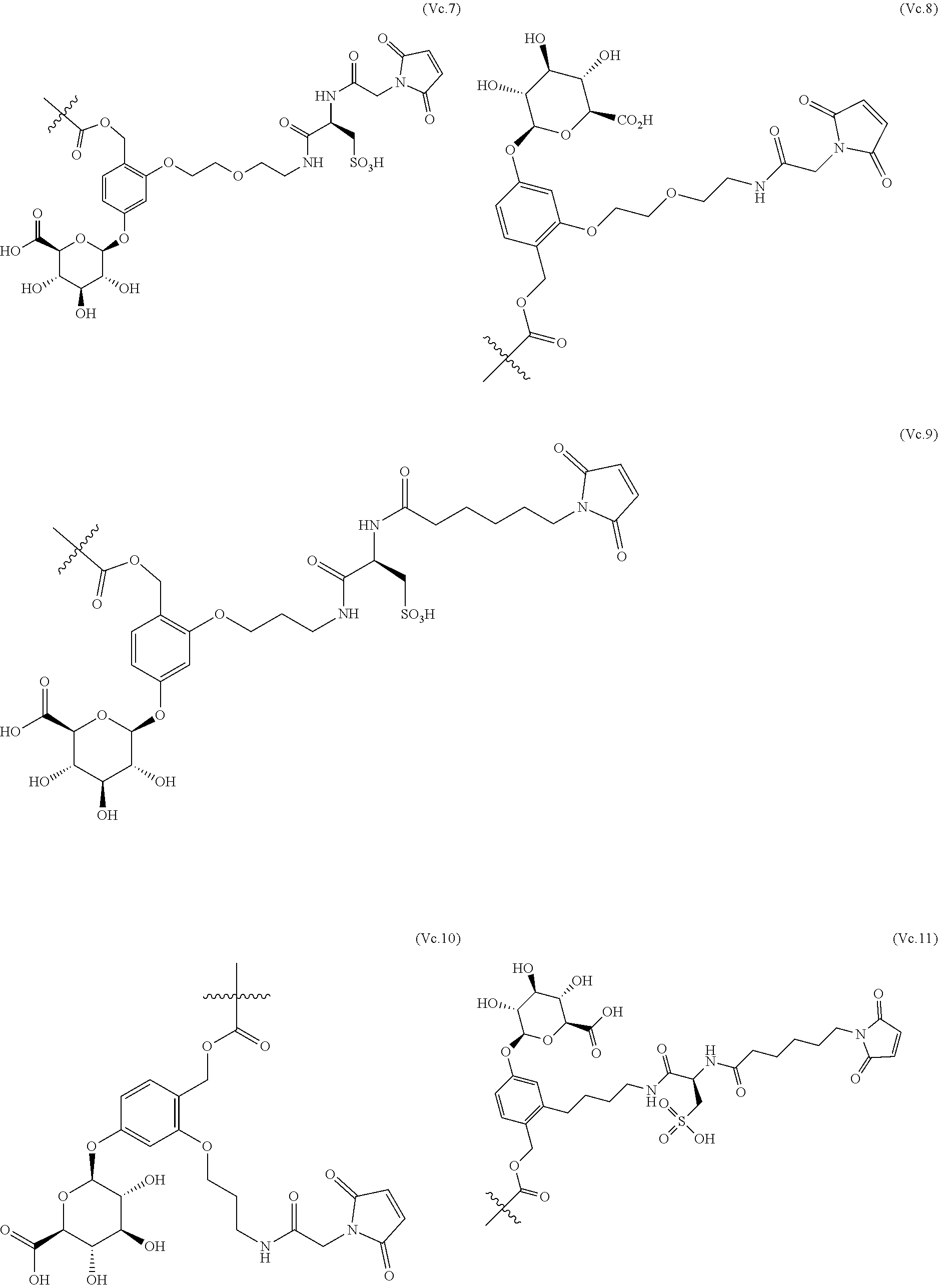

Exemplary embodiments of linkers according to structural formula (Vd) that may be included in the conjugates described herein include the linkers illustrated below (as illustrated, the linkers include a group suitable for covalently linking the linker in a conjugate and the wavy line indicates an attachment site for an immune-stimulatory compound):

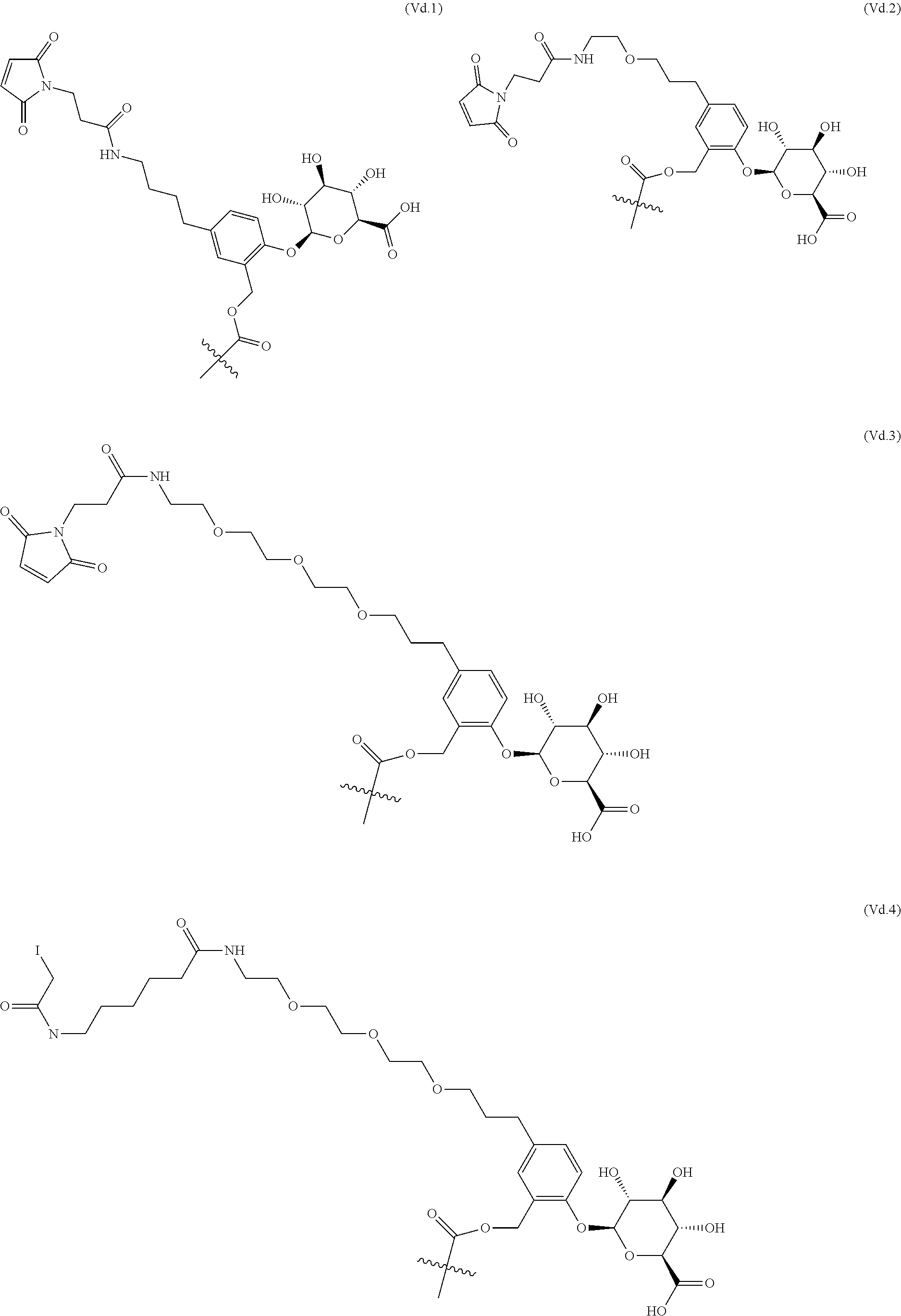 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCNC(=O)CI)ccc1O[C@@H]1O[C@H](C(=O)O)[C@@H](O)[C@H](O)[C@H]1O]

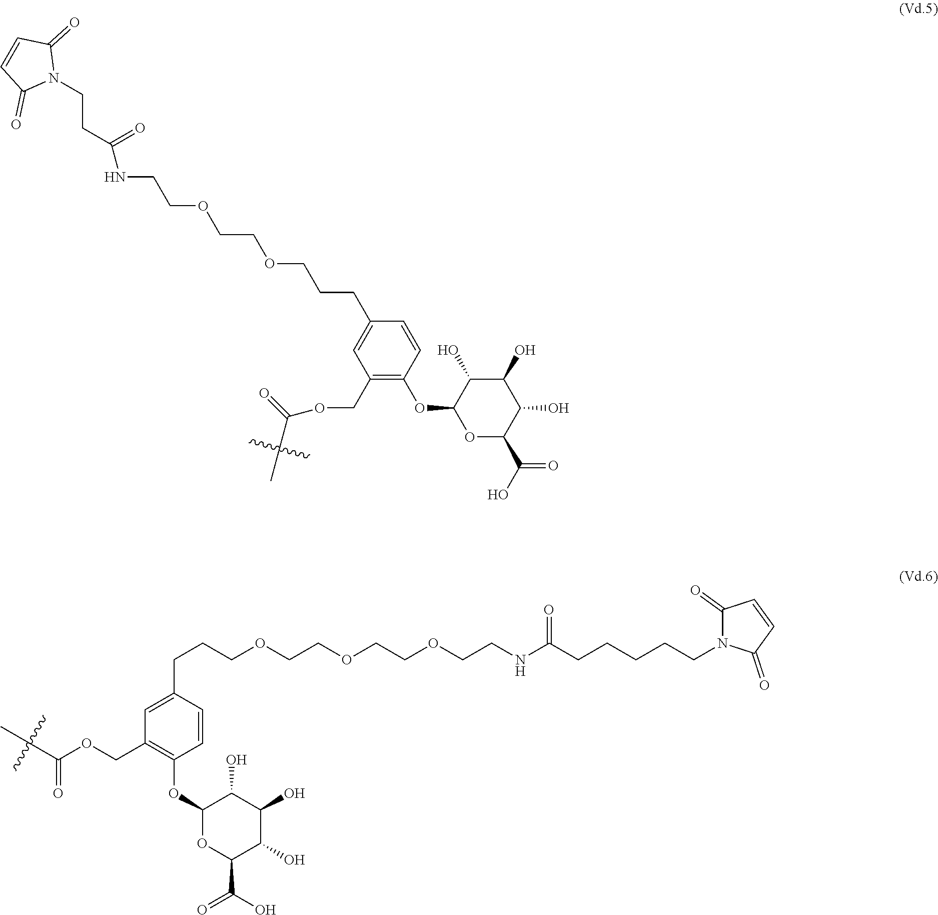 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of linkers according to structural formula (Ve) that may be included in the immune-stimulatory conjugates described herein include the linkers illustrated below (as illustrated, the linkers include a group suitable for covalently linking the linker in a conjugate and the wavy line indicates an attachment site for an immune-stimulatory compound):

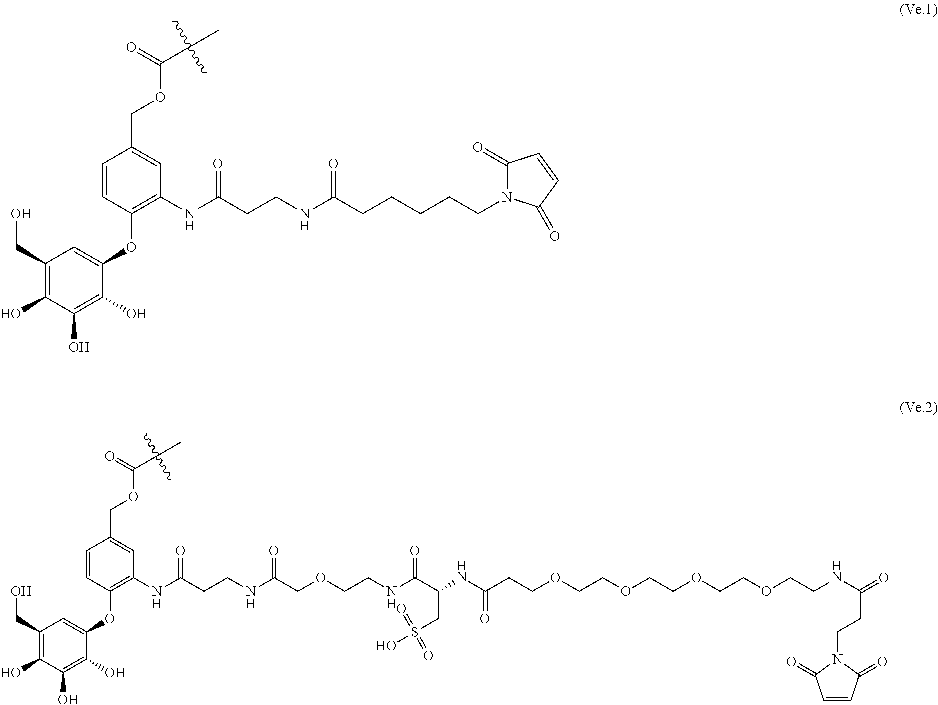

Although cleavable linkers can provide certain advantages, the linkers comprising the conjugate described herein need not be cleavable. For non-cleavable linkers, the immune-stimulatory compound release may not depend on the differential properties between the plasma and some cytoplasmic compartments. The release of the immune-stimulatory compound can occur after internalization of the conjugate via antigen-mediated endocytosis and delivery to lysosomal compartment, where the conjugate can be degraded to the level of amino acids through intracellular proteolytic degradation. This process can release an immune-stimulatory compound derivative, which is formed by the immune-stimulatory compound, the linker, or a portion thereof, and in some instances the amino acid residue to which the linker was covalently attached. The immune-stimulatory compound derivative from conjugates with non-cleavable linkers can be more hydrophilic and less membrane permeable, which can lead to less bystander effects and less nonspecific toxicities compared to conjugates with a cleavable linker. Conjugates with non-cleavable linkers can have greater stability in circulation than conjugates with cleavable linkers. Non-cleavable linkers can be alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

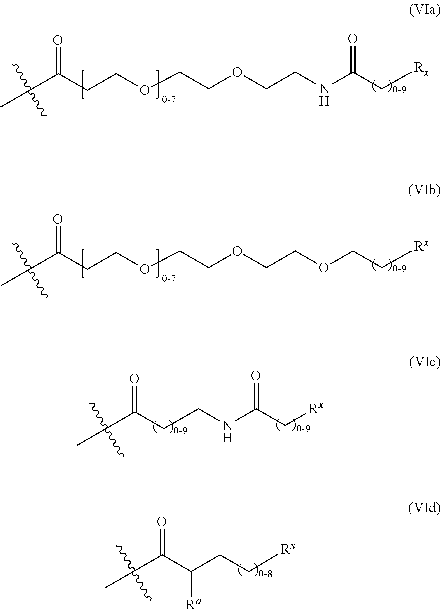

or salts thereof, wherein: R^{a} is selected from hydrogen, alkyl, sulfonate and methyl sulfonate; R^{x} is a moiety including a functional group capable of covalently linking the linker to an antibody construct of the conjugate; and [symbol] represents the point of attachment of the linker to the immune-stimulatory compound.

Exemplary embodiments of linkers according to structural formula (VIa)-(VId) that may be included in the conjugates described herein include the linkers illustrated below (as illustrated, the linkers include a group suitable for covalently linking the linker in a conjugate, and [symbol] represents the point of attachment in a conjugate):

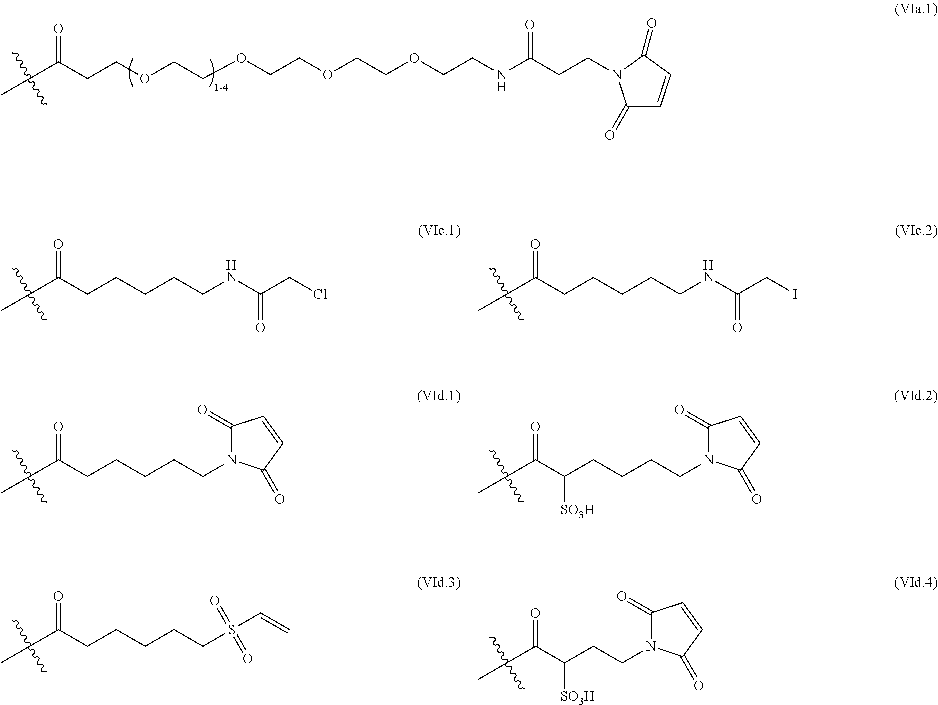

Attachment groups that are used to attach the linkers in a conjugate can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under conjugation conditions to give a conjugate species with improved stability is depicted in the schematic below. Thus, the maleimide attachment group is reacted with a sulfhydryl of a antibody construct to give an intermediate succinimide ring. The hydrolyzed (open ring) form of the attachment group is resistant to deconjugation in the presence of plasma proteins.

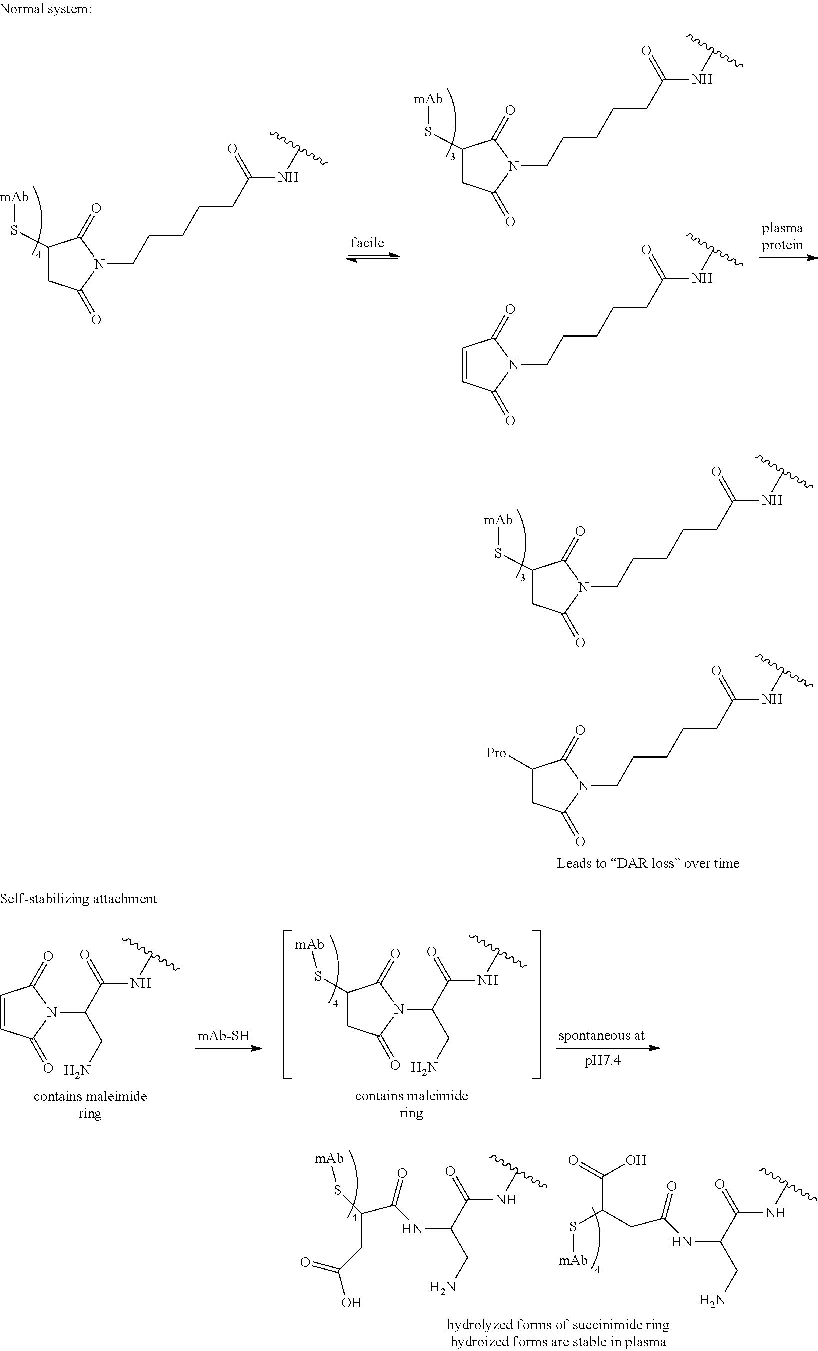

A method for bridging a pair of sulfhydryl groups derived from reduction of a native hinge disulfide bond has been disclosed and is depicted in the schematic below. An advantage of this methodology can be the ability to synthesize homogenous DAR4 conjugates by full reduction of IgGs (to give 4 pairs of sulfhydryls) followed by reaction with 4 equivalents of the alkylating agent. Conjugates containing “bridged disulfides” can also have increased stability.

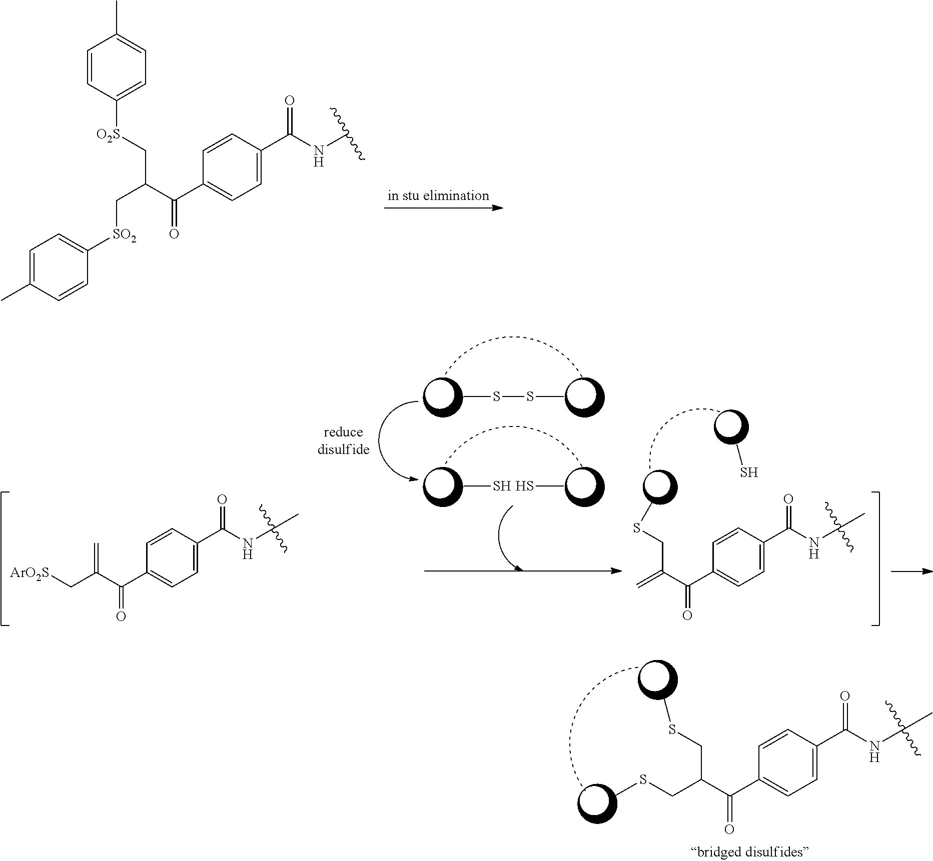

Similarly, as depicted below, a maleimide derivative that can bridge a pair of sulfhydryl groups has been developed.

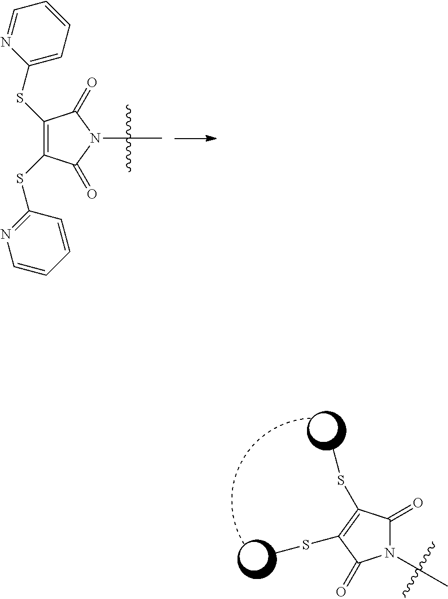 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIIa), (VIIb), or (VIIc):

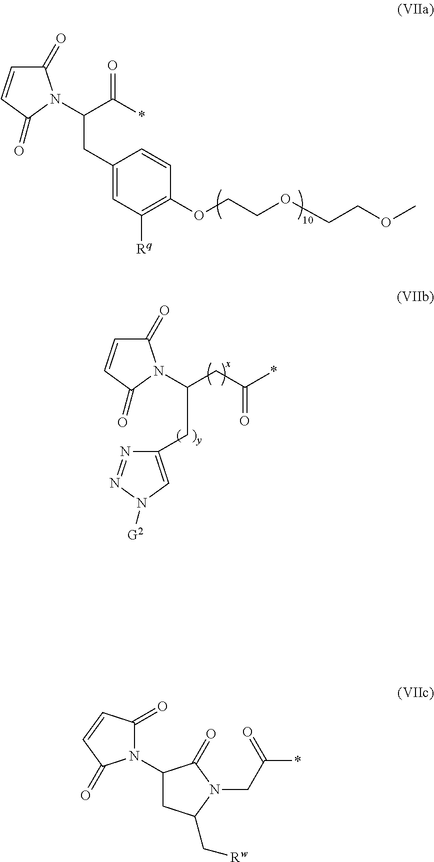

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIIa) and (VIIb) that can be included in the conjugates described herein can include the linkers illustrated below (as illustrated, the linkers can include a group suitable for covalently linking the linker in a conjugate and the wavy line or unlinked bond indicates an attachment site for an immune-stimulatory compound):

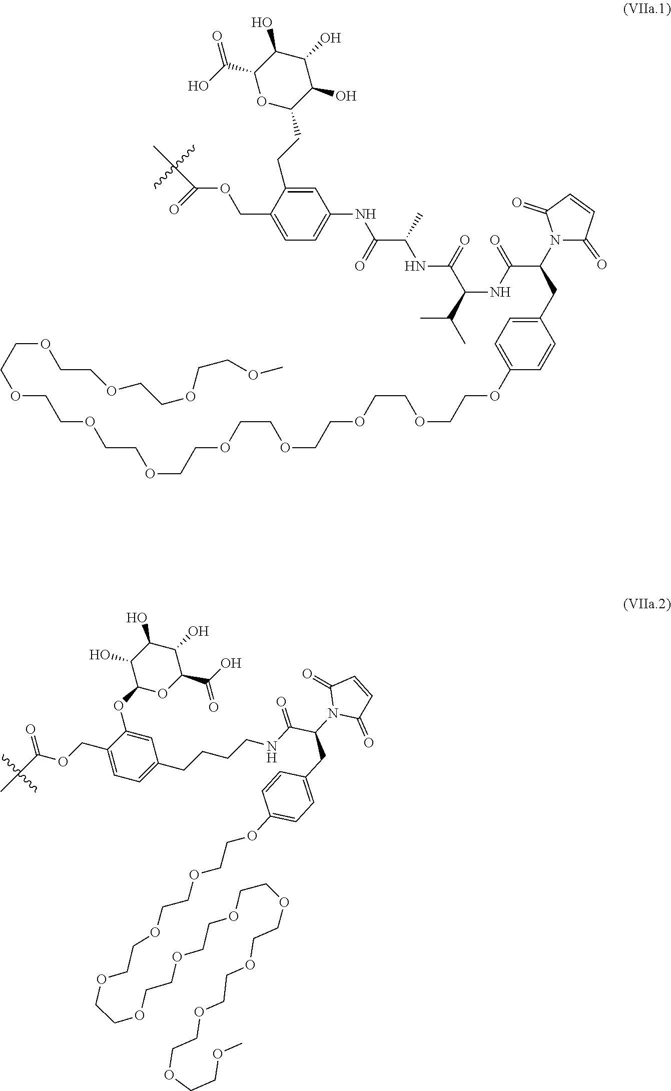 [SMILES: *C(=O)OCc1ccc(NC(=O)[C@H](C)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1CC[C@@H]1O[C@H](C(=O)O)[C@@H](O)[C@H](O)[C@H]1O ; *C(=O)OCc1ccc(CCCCNC(=O)[C@H](Cc2ccc(OCCOCCOCCOCCOCCOCCOCCOCCOCCOCCOCCOC)cc2)N2C(=O)C=CC2=O)cc1O[C@@H]1O[C@H](C(=O)O)[C@@H](O)[C@H](O)[C@H]1O]

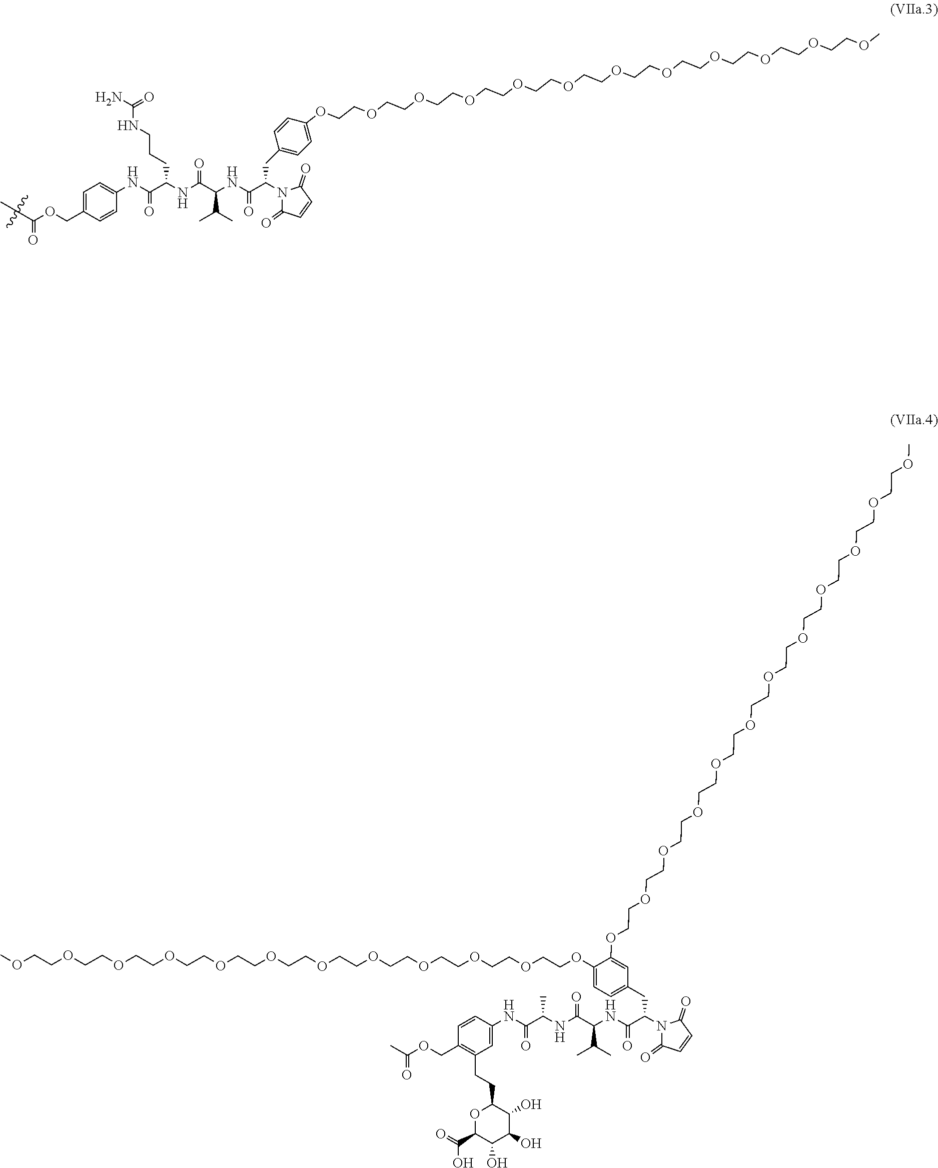

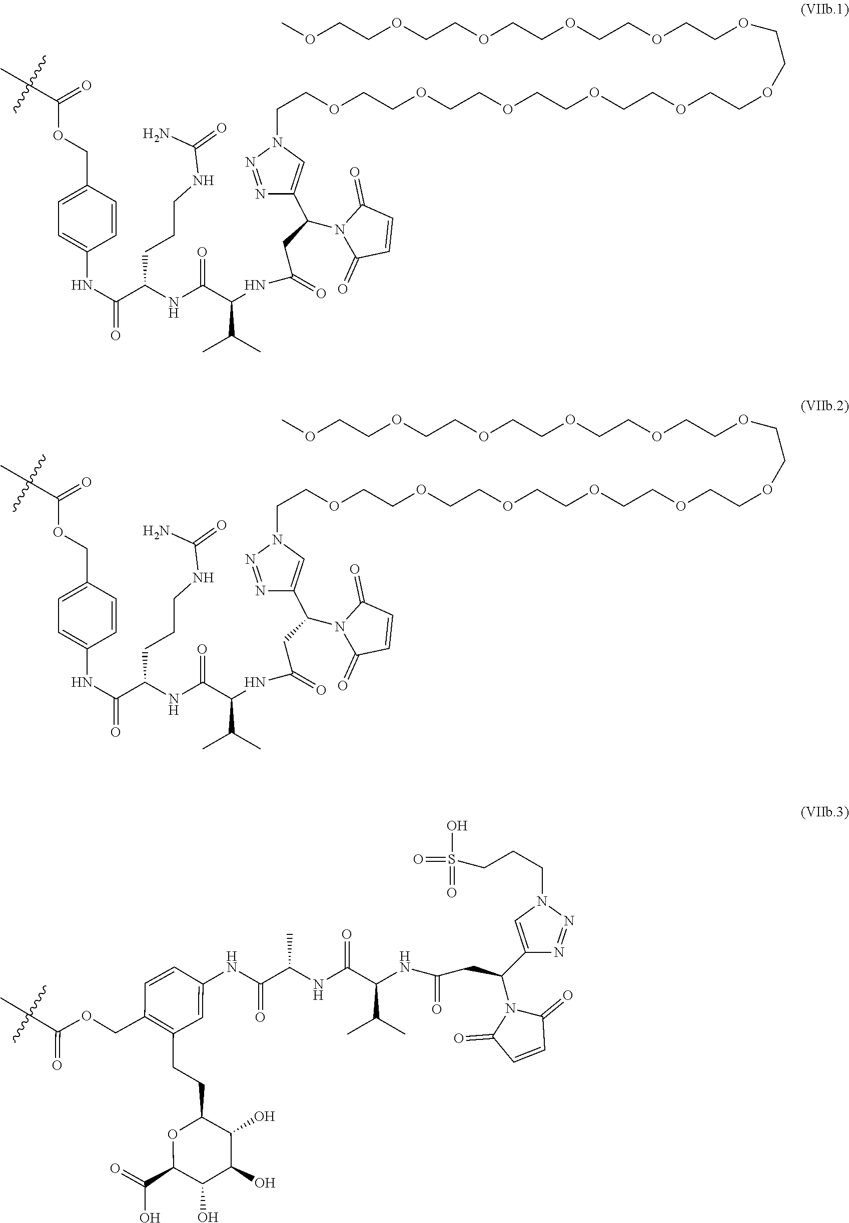 [SMILES: *C(=O)OCc1ccc(NC(=O)[C@H](CCCNC(N)=O)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](CCCNC(N)=O)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](C)NC(=O)[C@@H](NC(=O)C[C@@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O]

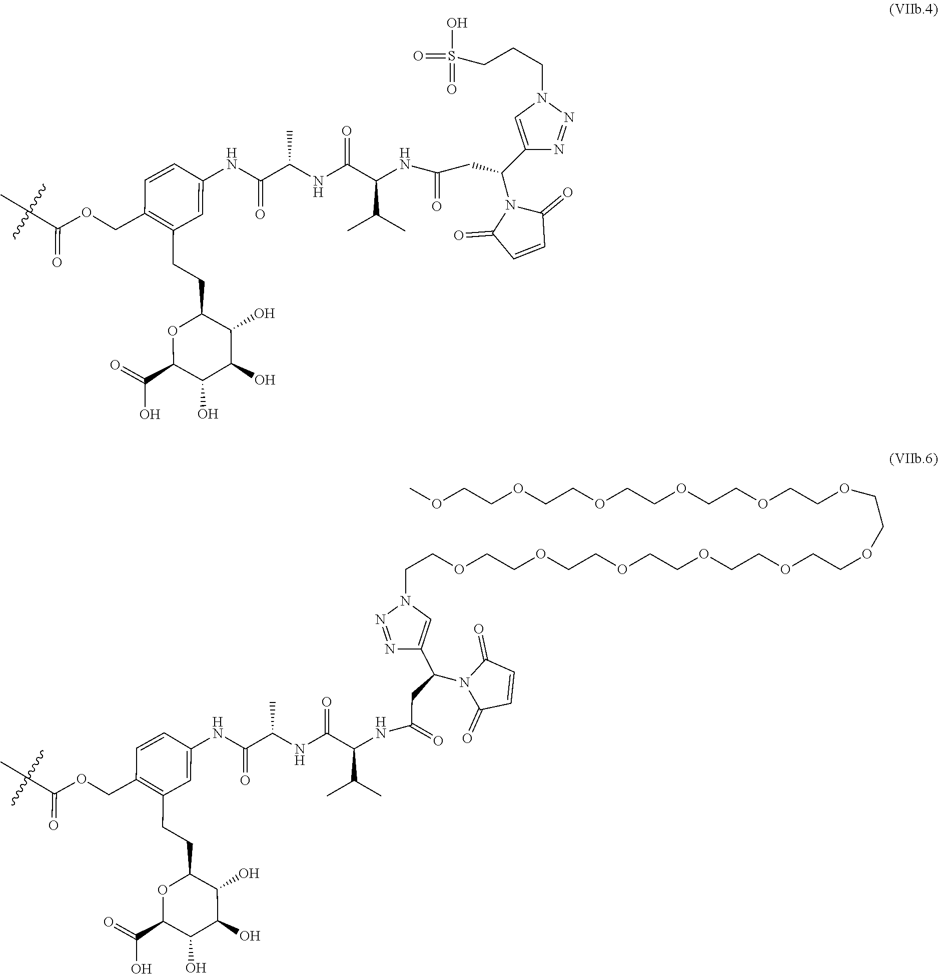 [SMILES: *C(=O)OCc1ccc(NC(=O)[C@H](C)NC(=O)[C@@H](NC(=O)C[C@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

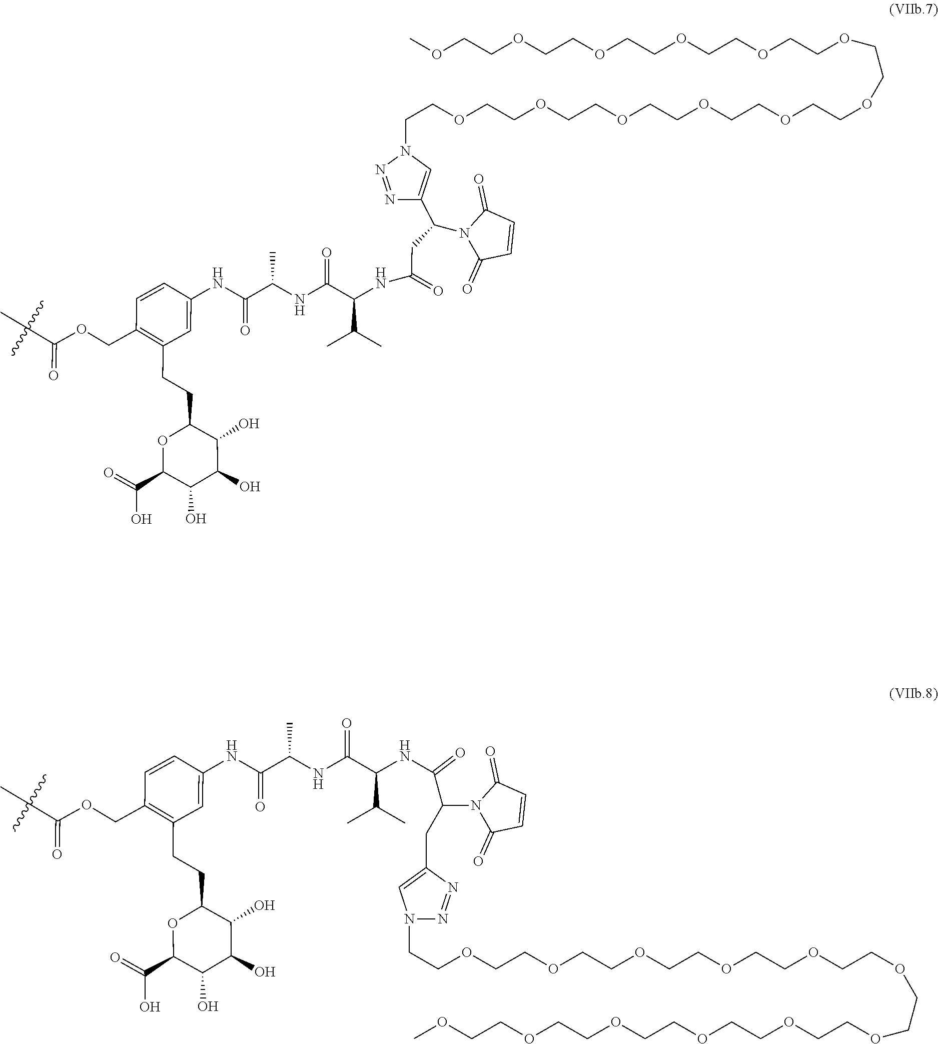 [SMILES: *C(=O)OCc1ccc(NC(=O)[C@H](C)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C(Cc2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

Exemplary embodiments of linkers according to structural formula (VIIc) that can be included in the conjugates described herein can include the linkers illustrated below (as illustrated, the linkers can include a group suitable for covalently linking the linker in a conjugate):

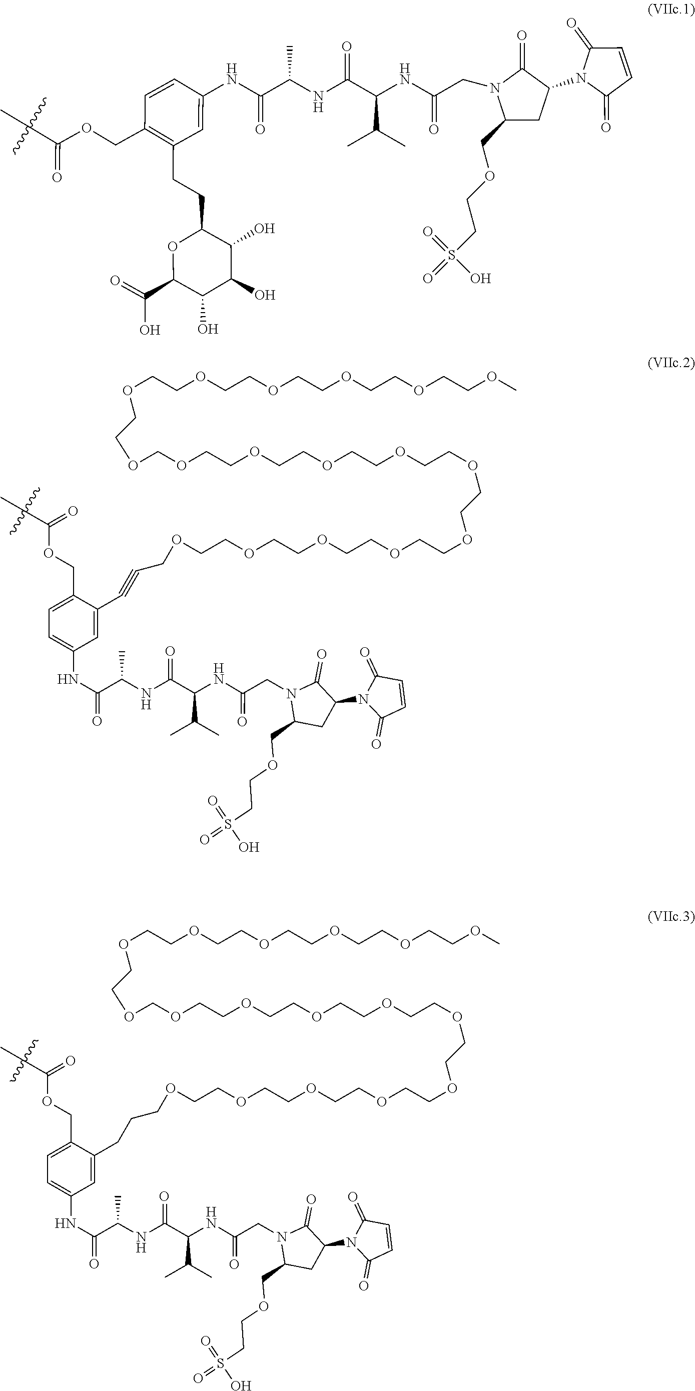 [SMILES: *C(=O)OCc1ccc(NC(=O)[C@H](C)NC(=O)[C@@H](NC(=O)CN2C(=O)[C@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1C#CCOCCOCCOCCOCCOCCOCCOCCOCCOCCOCOCCOCCOCCOCCOCCOCCOC ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CCCOCCOCCOCCOCCOCCOCCOCCOCCOCCOCOCCOCCOCCOCCOCCOCCOC]

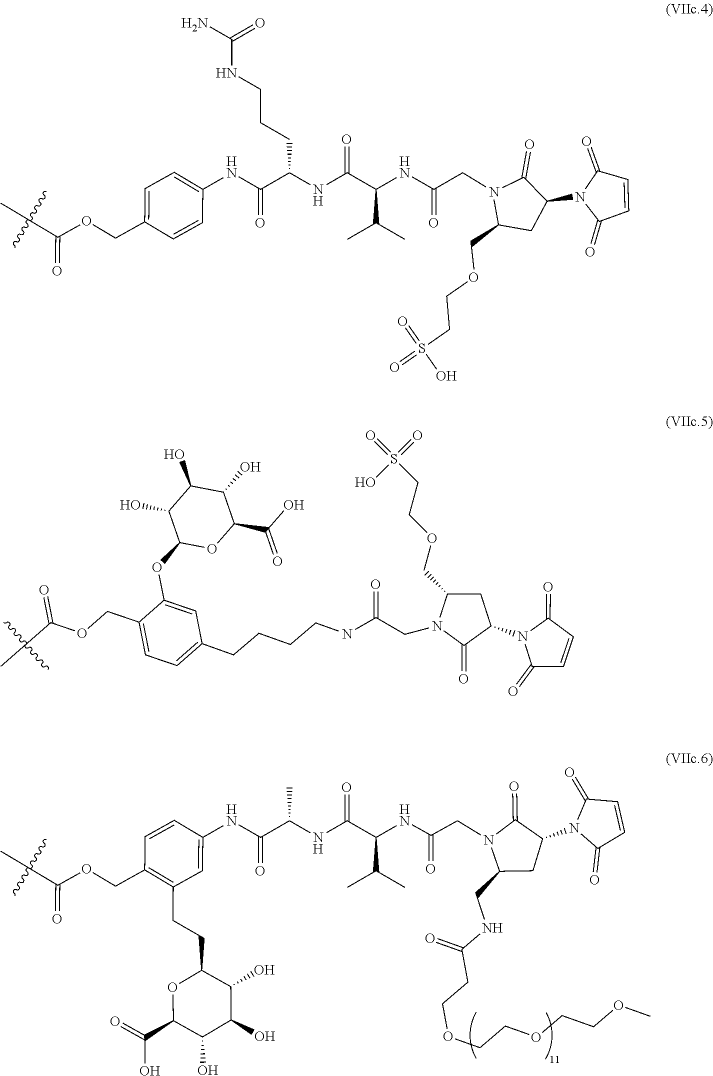

### Conjugates

A conjugate as described herein can comprise an antibody construct or a recombinant bispecific antibody and at least one linker connected to an immune-stimulatory compound. As will be appreciated by the skilled artisan, the following description of conjugates comprising antibody constructs is applicable to conjugates comprising recombinant bispecific antibodies.

In some aspects, the present disclosure provides a conjugate comprising an immune-stimulatory compound or salt thereof, an antibody construct, and a linker, wherein the compound or salt is linked, i.e., covalently bound, to the antibody construct through the linker. The linker can be selected from a cleavable or non-cleavable linker. In some embodiments, the linker is cleavable. In alternative embodiments, the linker is non-cleavable. Linkers are further described in the present application in the subsequent section, any one of which can be used to connect an antibody to a compound described herein.

In a conjugate, the drug loading is represented by p, the number of immune-stimulatory compound-linker molecules per antibody construct, or the number of immune-stimulatory compounds per antibody construct, depending on the particular conjugate. Depending on the context, p can represent the average number of immune-stimulatory compounds (-linker) molecules per antibody construct, also referred to the average drug loading. P can range from 1 to 20, from 1-50 or from 1-100. In some conjugates, p is preferably from 1 to 8. In some preferred embodiments, when p represents the average drug loading, p ranges from about 2 to about 5. In some embodiments, p is about 2, about 3, about 4, or about 5. The average number of immune-stimulatory compounds per antibody construct in a preparation may be characterized by conventional means such as mass spectroscopy, HIC, ELISA assay, and HPLC.

A conjugate can comprise an antibody construct, an immune-stimulatory compound, and a linker. A conjugate can comprise an antibody construct, a pattern recognition receptor (PRR) agonist, and a linker. A conjugate can comprise an antibody construct, a pattern-associated molecular pattern (PAMP) molecule, and a linker. A conjugate can comprise an antibody construct, a damage-associated molecular pattern (DAMP) molecule, and a linker. A conjugate can comprise an antibody construct, a STING agonist, and a linker. A conjugate can comprise an antibody construct, a toll-like receptor agonist molecule, and a linker. A conjugate can comprise an antibody construct, imiquimod, and a linker. A conjugate can comprise an antibody construct, S-27609, and a linker. A conjugate can comprise an antibody construct, CL307, and a linker. A conjugate can comprise an antibody construct, resiquimod, and a linker. A conjugate can comprise an antibody construct, gardiquimod, and a linker. A conjugate can comprise an antibody construct, UC-IV150, and a linker. A conjugate can comprise an antibody construct, KU34B, and a linker. A conjugate can comprise an antibody construct, motolimod, and a linker. A conjugate can comprise an antibody construct, VTX-1463, and a linker. A conjugate can comprise an antibody construct, GS-9620, and a linker. A conjugate can comprise an antibody construct, GSK2245035, and a linker. A conjugate can comprise an antibody construct, TMX-101, and a linker. A conjugate can comprise an antibody construct, TMX-201, and a linker. A conjugate can comprise an antibody construct, TMX-202, and a linker. A conjugate can comprise an antibody construct, isatoribine, and a linker. A conjugate can comprise an antibody construct, AZD8848, and a linker. A conjugate can comprise an antibody construct, MEDI9197, and a linker. A conjugate can comprise an antibody construct, 3M-051, and a linker. A conjugate can comprise an antibody construct, 3M-852, and a linker. A conjugate can comprise an antibody construct, 3M-052, and a linker. A conjugate can comprise an antibody construct, 3M-854A, and a linker. A conjugate can comprise an antibody construct, S-34240, and a linker. A conjugate can comprise an antibody construct, CL663, and a linker. A conjugate can comprise an antibody construct, KIN1148, and a linker. A conjugate can comprise an antibody construct, SB-9200, and a linker. A conjugate can comprise an antibody construct, KIN-100, and a linker. A conjugate can comprise an antibody construct, ADU-S100, and a linker. A conjugate can comprise an antibody construct, KU34B, and a linker.

A conjugate described herein can have a native Fc domain. A conjugate described herein can have a modified Fc domain. The modified Fc domain can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A. The numbering of amino acids residues described herein can be according to the EU index.

The linker can be a linker as described herein. A linker can be cleavable, non-cleavable, hydrophilic, or hydrophobic. A cleavable linker can be sensitive to enzymes. A cleavable linker can be cleaved by enzymes such as proteases. A cleavable linker can be a linker containing a valine-citrulline or a valine-alanine peptide. A valine-citrulline- or valine-alanine-containing linker can contain a pentafluorophenyl group. A valine-citrulline- or valine-alanine-containing linker can contain a succimide group. A valine-citrulline- or valine-alanine-containing linker can contain a PABA group. A valine-citrulline- or valine-alanine-containing linker can contain a PABA group and a pentafluorophenyl group. A valine-citrulline-containing or valine-alanine-containing linker can contain a PABA group and a maleimide group. A valine-citrulline-containing or valine-alanine-containing linker can contain a PABA group and a succinimide group. A non-cleavable linker can be protease insensitive. A non-cleavable linker can contain a maleimide group. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimide group and one or more polyethylene glycol molecules. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can be a THIOMAB linker. A THIOMAB linker can be a (maleimidocaproyl)-(valine-citrulline)-(para-aminobenzyloxycarbonyl) linker. A linker can also comprise an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 672) to an N-terminal GGG motif to regenerate a native amide bond. The linker created can therefore link a moiety attached to the LXPTG recognition motif (SEQ ID NO: 672) with a moiety attached to the N-terminal GGG motif. A linker can be a link created between an unnatural amino acid on one moiety reacting with oxime bond that was formed by modifying a ketone group with an alkoxyamine on another moiety. A moiety can be an antibody construct. A moiety can be a binding domain. A moiety can be an antibody. A moiety can be an immune-stimulatory compound.

A conjugate can be an anti-tumor antigen conjugate. The conjugate can comprise an anti-tumor antigen antibody or antibody construct. An antigen recognized by the conjugate can be CD5, CD19, CD20, CD25, CD37, CD30, CD33, CD45, CAMPATH-1, BCMA, CS-1, PD-L1, B7-H3, B7-DC, HLA-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, EGRFvIII (de2-7), EGFR, fibroblast activation protein, tenascin, a metalloproteinase, endosialin, vascular endothelial growth factor, αvβ3, WT1, LMP2, HPV E6, HPV E7, Her-2/neu, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, PAGE4, VEGFR2, MAD-CT-1, PDGFR-B, MAD-CT-2, ROR2, TRAIL1, MUC16, MAGE A4, MAGE C2, GAGE, EGFR, CMET, HER3, CA6, NAPI2B, TROP2, Claudin-6 (CLDN6), Claudin-16 (CLDN16), CLDN18.2, RON, LY6E, FRA, DLL3, PTK7, Uroplakin-IB (UPK1B), VTCN1 (B7-H4), STRA6, TMPRSS3, TMRRSS4, TMEM238, C1orf186, LIV1, ROR1, Fos-related antigen 1, VEGFR1, endoglin, LRRC15, VISTA, or a fragment thereof. The conjugate can recognize an antigen that can be expressed on a cell. The conjugate can recognize an antigen that can be expressed by a cell. The conjugate can recognize an antigen that can be expressed in the context of a Major Histocompatibility Complex. The conjugate can recognize an antigen that can stimulate activity of a cell. The conjugate can recognize an antigen that can stimulate an immune response. The conjugate can recognize an antigen that can reduce an immune response. The conjugate can recognize an antigen that can reduce activity of a cell. The conjugate can recognize an antigen that can be expressed on an immune cell. The conjugate can recognize an antigen that can be expressed by an immune cell. The conjugate can recognize an antigen that can be in the context of a Major Histocompatibility Complex. The conjugate can recognize an antigen on a cell wherein the antigen can be involved in stimulating activity of a cell. The conjugate can recognize an antigen on an immune cell that can be involved in the costimulation of an immune cell. The conjugate can recognize an antigen on an immune cell that can be involved in the costimulation of an immune cell during an immune response. The conjugate can recognize a receptor. The conjugate can recognize a receptor on a cell. The conjugate can recognize a receptor ligand. The conjugate can recognize a receptor on a cell wherein the receptor can be involved in stimulating activity of a cell. The conjugate can recognize a receptor on an immune cell. The conjugate can recognize a receptor on an immune cell that can be involved in stimulating activity of an immune cell. The conjugate can recognize a receptor on an immune cell that can be involved in the costimulation of an immune cell. The conjugate can recognize a receptor on an immune cell that can be involved in the costimulation of an immune cell during an immune response. The conjugate can recognize an antigen that can be expressed on an immune cell and that can stimulate activity of an immune cell. The conjugate can recognize an antigen that can be expressed on an immune that can reduce activity of an immune cell. The conjugate can be an anti-CD40 antibody. The conjugate can comprise a light chain of an SBT-040 antibody. The conjugate can comprise an SBT-040-G1WT heavy chain. The conjugate can comprise an SBT-040-G1VLPLL heavy chain. The conjugate can comprise an SBT-040-G1DE heavy chain. The conjugate can comprise an SBT-040-G1AAA heavy chain. The conjugate can comprise an SBT-040-CDR sequence.

The conjugate can be capable of recognizing a single antigen. The conjugate can be capable of recognizing two or more antigens. The conjugate can be capable of recognizing three or more antigens. The Kd for binding of a second binding domain of a conjugate to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the second binding domain to the antigen of a conjugate in the absence of the immune-stimulatory compound. The Kd for binding of a second binding domain of a conjugate to an antigen in the presence of the immune-stimulatory compound can be less than 10 nM. The Kd for binding of a second binding domain of a conjugate to an antigen in the presence of the immune-stimulatory compound can be less than 100 nM, less than 50 nM, less than 20 nM, less than 5 nM, less than 1 nM, or less than 0.1 nM. In contrast, the Kd for binding of a second binding domain of conjugate to an antigen in the presence of the immune-stimulatory compound when the first binding domain is bound to the first binding domain's antigen can be greater than 100 nM. The Kd for binding of a second binding domain of a conjugate to an antigen in the presence of the immune-stimulatory compound when the first binding domain is bound to the first binding domain's antigen can be greater than 100 nM, greater than 200 nM, greater than 300 nM, greater than 400 nM, greater than 500 nM, or greater than 1000 nM. The Kd for binding of a first binding domain of a conjugate to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the first binding domain to the antigen of a conjugate in the absence of the immune-stimulatory compound. The Kd for binding of a first binding domain of a conjugate to an antigen in the presence of the immune-stimulatory compound can be less than 10 nM. The Kd for binding of a first binding domain of a conjugate to an antigen in the presence of the immune-stimulatory compound can be less than 100 nM, less than 50 nM, less than 20 nM, less than 5 nM, less than 1 nM, or less than 0.1 nM.

The conjugate can comprise a binding domain. A binding domain of a conjugate can recognize an antigen. For example, an antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be CD40 and a binding domain can recognize a CD40 antigen. A binding domain of a conjugate can be a CD40 agonist.

The conjugate can comprise an Fc domain that can bind to an FcR when linked to an immune-stimulatory compound. The conjugate can comprise an Fc domain that can bind to an FcR to initiate FcR-mediated signaling when linked to an immune stimulatory compound. The conjugate can bind to its antigen when linked to an immune-stimulatory compound. The conjugate can bind to its antigen when linked to an immune-stimulatory compound and the Fc domain of the conjugate can bind to an FcR when linked to an immune-stimulatory compound. The conjugate can bind to its antigen when linked to an immune-stimulatory compound and the Fc domain of the conjugate can bind to an FcR to initiate FcR-mediated signaling when linked to an immune stimulatory compound. The Fc domain linked to an immune-stimulatory compound can be a modified Fc domain. The modified Fc domain can comprise a substitution at more than one amino acid residue, such as at 5 different amino acid residues including L235V/F243L/R292P/Y300L/P396L, as at 2 different amino acid residues including S239D/I332E, or as at 3 different amino acid residues including S298A/E333A/K334A. The Kd for binding of an Fc domain to a Fc receptor when the Fc domain is linked to an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the Fc domain to the Fc receptor in the absence of linking to the immune-stimulatory compound. The Kd for binding of an Fc domain to an Fc receptor when linked to an immune-stimulatory compound can be less than 10 nM. The Kd for binding of an Fc domain to an Fc receptor when linked to an immune-stimulatory compound can be less than 100 nM, less than 50 nM, less than 20 nM, less than 5 nM, less than 1 nM, or less than 0.1 nM. In contrast, the Kd for binding of an Fc domain to an Fc receptor when linked to an immune-stimulatory compound and when the first binding domain is bound to its antigen can be greater than 100 nM. The Kd for binding of an Fc domain to an Fc receptor when linked to an immune-stimulatory compound and when the first binding domain is bound to its antigen can be greater than 100 nM, greater than 200 nM, greater than 300 nM, greater than 400 nM, greater than 500 nM, or greater than 1000 nM.

The binding domain can be selected in order to recognize an antigen. For example, an antigen can be expressed on an immune cell. An antigen can be expressed on a T cell, a B cell, an NKT cell, or an NK cell. An antigen can be a peptide or fragment thereof. An antigen can be expressed on an antigen-presenting cell. An antigen can be expressed on a dendritic cell, a macrophage, or a B cell. An antigen can be CD40 and a binding domain can recognize a CD40 antigen. A binding domain can be a CD40 agonist. A binding domain can be CD40.

The immune-stimulatory compound of the conjugate can be a PRR agonist. The PRR agonist can be a toll-like receptor agonist. The toll-like receptor agonist can be a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, a TLR12 agonist or a TLR13 agonist. The toll-like receptor agonist can activate two or more TLRs. The immune-stimulatory compound of the conjugate can be a PAMP molecule. The PAMP molecule can be a RIG-I agonist.

A conjugate can comprise an antibody construct, KU34B, and a linker. An antibody construct of any of the conjugates described herein can have a modified Fc domain of the antibody construct. The modified Fc domain can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A. The numbering of amino acids residues described herein can be according to the EU index.

A conjugate can be formed by a linker that can connect an antibody construct to an immune-stimulatory compound. A conjugate can be formed by a linker that can connect an antibody construct to a PRR molecule. A conjugate can be formed by a linker that can connect an antibody construct to a PAMP molecule. A conjugate can be formed by a linker that can connect an antibody construct and a DAMP molecule. A conjugate can be formed by a linker that can connect an antibody construct to a PRR, and a linker that can connect an antibody construct and a binding domain. A conjugate can be formed by a linker that can connect an antibody construct to a PAMP molecule, and a linker that can connect an antibody construct and a binding domain. A conjugate can be formed by a linker that can connect an antibody construct and a DAMP molecule, and a linker that can connect an antibody construct and a binding domain.

A linker can be connected to an antibody construct of a conjugate by a direct linkage between the antibody construct and the linker. A linker can be connected to an anti-CD40 antibody construct by a direct linkage between the anti-CD40 antibody construct and the linker. A linker can be connected to an anti-CD40 antibody by a direct linkage between the anti-CD40 antibody and the linker. A linker can be connected to an anti-tumor antigen antibody construct by a direct linkage between the anti-tumor antigen antibody construct and the linker. A linker can be connected to an anti-tumor antigen antibody by a direct linkage between the anti-tumor antigen antibody and the linker. A direct linkage is a covalent bond.

A linker can be attached to an antibody construct at any suitable site, such as for example at a terminus of an amino acid sequence or at a side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment site can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO: 672) to an N-terminal GGG motif to regenerate a native amide bond. The linker created can therefore link a moiety attached to the LXPTG recognition motif (SEQ ID NO: 672) with a moiety attached to the N-terminal GGG motif.

An attachment can be via any of a number of bonds, for example but not limited to, an amide bond, an ester bond, an ether bond, a carbon-nitrogen bond, a carbon-carbon single, double or triple bond, a disulfide bond, or a thioether bond. A linker can have at least one functional group, which can be linked to the antibody construct or the antibody. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

A linker can be connected to an antibody construct at a hinge cysteine. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to an antibody construct at a heavy chain constant domain lysine. A linker can be connected to an antibody construct at an engineered cysteine in the heavy chain. A linker can be connected to an antibody construct at an engineered heavy chain glutamine. A linker can be connected to an antibody construct an unnatural amino acid engineered into the heavy chain. Amino acids can be engineered into an amino acid sequence of an antibody construct as described herein, for example, and can be connected to a linker of a conjugate. Engineered amino acids can be added to a sequence of existing amino acids. Engineered amino acids can be substituted for one or more existing amino acids of a sequence of amino acids.

A linker can be conjugated to an antibody construct via a sulfhydryl group. A linker can be conjugated to an antibody construct via a primary amine. A linker can be a link created between an unnatural amino acid on an antibody construct reacting with oxime bond that was formed by modifying a ketone group with an alkoxyamine on an immune-stimulatory compound. When a linker is connected to an antibody construct at the sites described herein, an Fc domain of the conjugate can bind to Fc receptors. When a linker is connected to an antibody construct at the sites described herein, the antigen binding domain of the conjugate can bind its antigen. When a linker is connected to an antibody construct at the sites described herein, a binding domain of the conjugate can bind its antigen.

An antibody with engineered reactive cysteine residues can be used to link a binding domain to the antibody. A linker can connect an antibody construct to a binding domain via Sortase A linker. A Sortase A linker can be created by a Sortase A enzyme fusing an LXPTG recognition motif (SEQ ID NO: 672) to an N-terminal GGG motif to regenerate a native amide bond. The linker created can therefore link an antibody construct attached to the LXPTG recognition motif (SEQ ID NO: 672) with a binding domain attached to the N-terminal GGG motif. A binding domain can be connected to a linker by a direct linkage. A direct linkage is a covalent bond. For example, a linker can be attached to a terminus of an amino acid sequence of a binding domain, or could be attached to a side chain modification to the binding domain, such as the side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the binding domain. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the binding domain. Engineered amino acids can be added to a sequence of existing amino acids. Engineered amino acids can be substituted for one or more existing amino acids of a sequence of amino acids. A linker can be conjugated to a binding domain via a sulfhydryl group. A linker can be conjugated to a binding domain via a primary amine. A binding domain can be conjugated to the C-terminal of an Fc domain of a conjugate.

An antibody or antibody construct with engineered reactive cysteine residues can be used to link an immune-stimulatory compound to the antibody or antibody construct. A linker can connect an antibody construct to an immune-stimulatory compound via linker. A linker can connect an antibody construct to an immune-stimulatory compound via Sortase A linker. A Sortase A linker can be created by a Sortase A enzyme fusing an LXPTG recognition motif (SEQ ID NO: 672) to an N-terminal GGG motif to regenerate a native amide bond. The linker created can therefore link an antibody attached the LXPTG recognition motif (SEQ ID NO: 672) with an immune-stimulatory compound attached to the N-terminal GGG motif. A linker can be a link created between an unnatural amino acid an antibody reacting with oxime bond that was formed by modifying a ketone group with an alkoxyamine on an immune-stimulatory compound. The immune-stimulatory compound can comprise one or more rings selected from carbocyclic and heterocyclic rings. The immune-stimulatory compound can be covalently bound to a linker by a bond to an exocyclic carbon or nitrogen atom on the immune-stimulatory compound. A linker can be conjugated to an immune-stimulatory compound via an exocyclic nitrogen or carbon atom of an immune-stimulatory compound. A linker can be connected to a STING agonist, for example:

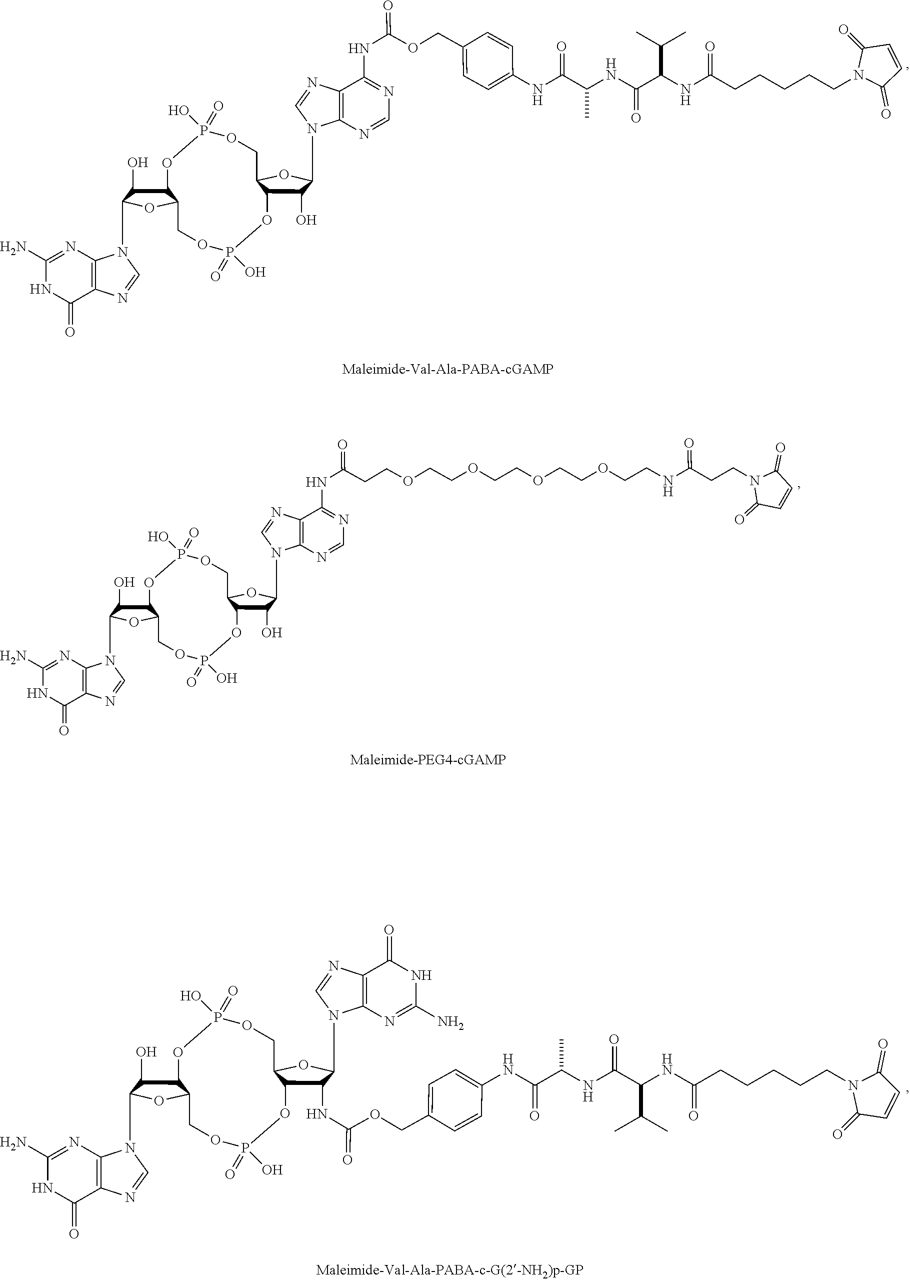

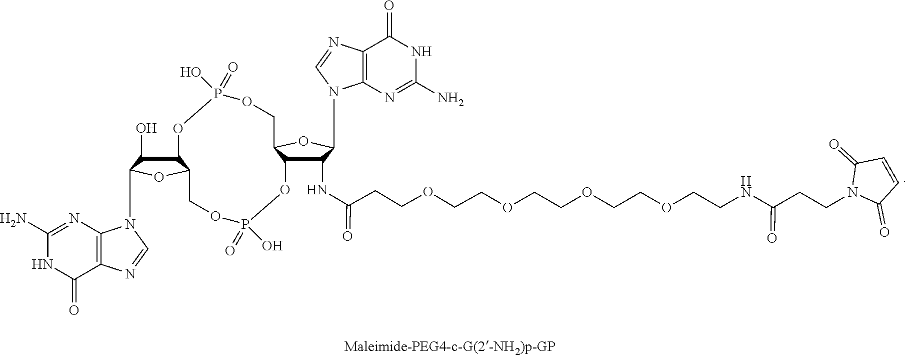

A linker agonist complex can dissociate under physiological conditions to yield an active agonist.

A linker can be connected to a PRR agonist by a direct linkage between the PRR agonist and the linker. A linker can be connected to a PAMP molecule by a direct linkage between the PAMP molecule and the linker. A linker can be connected to a toll-like receptor agonist by a direct linkage between the toll-like receptor agonist and the linker.

Examples of toll-like receptor agonists connected to a linker in a manner able to release an active toll-like receptor agonist under physiologic conditions can include:

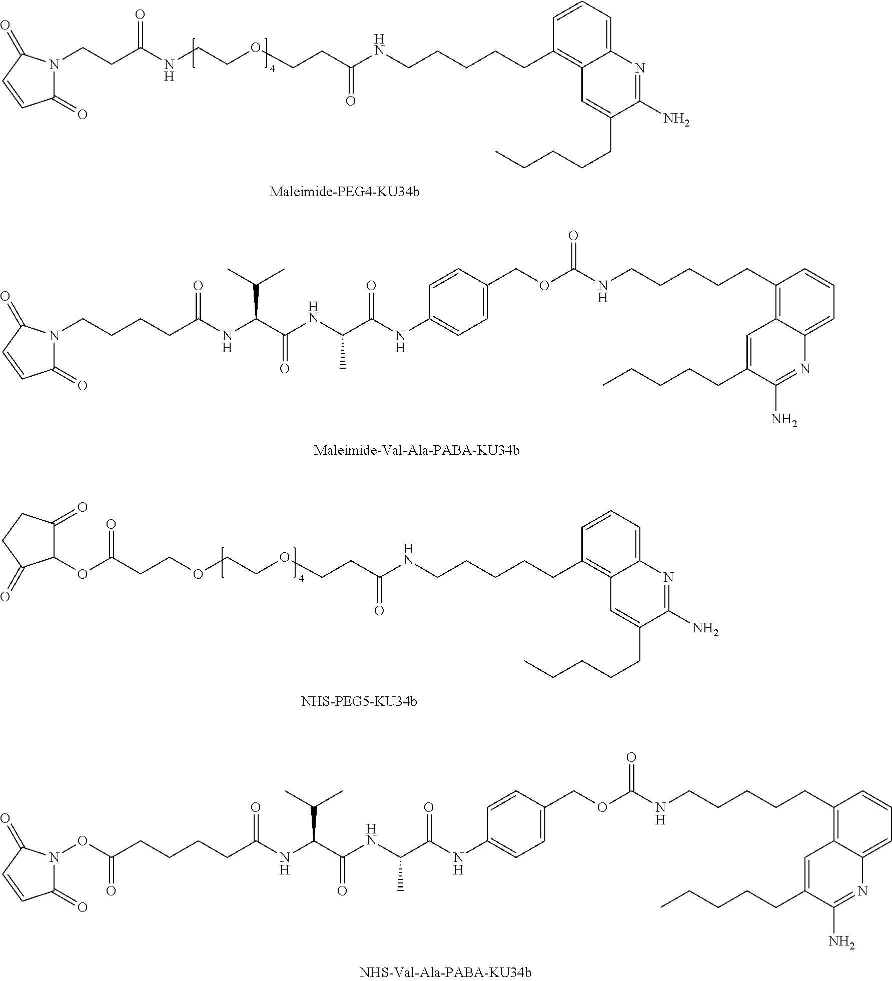

Examples of RIG-I agonists connected to a linker in a manner able to release an active toll-like receptor agonist under physiologic conditions can include:

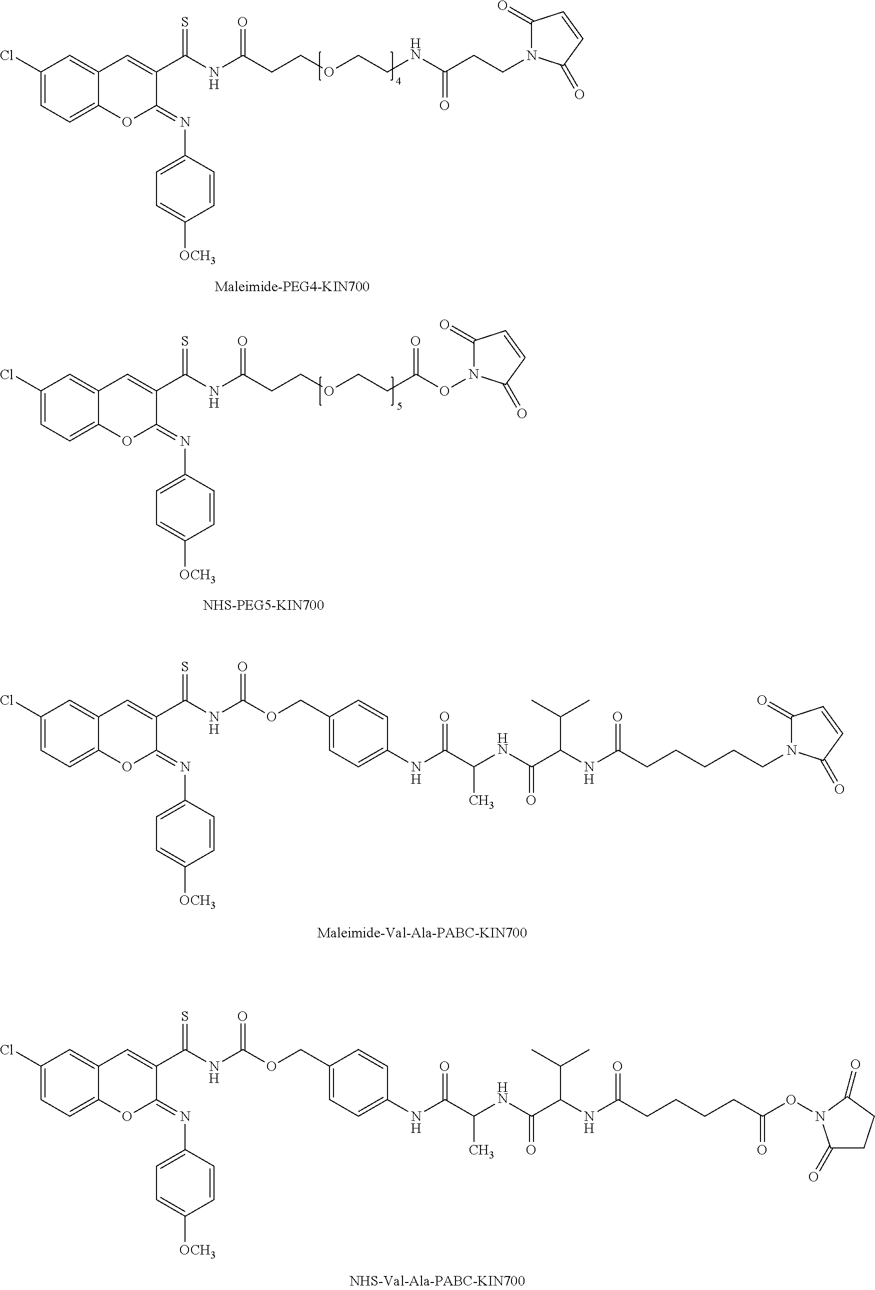

A linker can be connected to a DAMP molecule by a direct linkage between the DAMP molecule and the linker. A direct linkage can be a covalent bond. For example, a linker can be attached to a terminus of an amino acid sequence of an antibody or antibody construct, or could be attached to a side chain modification to the antibody or antibody construct, such as example at a side chain of a cysteine residue, an engineered cysteine residue, a lysine residue, a serine residue, a threonine residue, a tyrosine residue, an aspartic acid residue a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO: 672) to an N-terminal GGG motif to regenerate a native amide bond. The linker created can therefore link a moiety attached to the LXPTG recognition motif (SEQ ID NO: 672) with a moiety attached to the N-terminal GGG motif. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody construct. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

In some embodiments, the linker is not attached to an amino acid residue of the Fc domain of the antibody construct selected from a group consisting of: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in said Fc domain is according to the EU index as in Kabat. In some embodiments, the linker is not attached to an amino acid residue of the Fc domain of the antibody construct selected from a group consisting of: 221, 224, 227, 230, 231, 232, 234, 235, 236, 237, 239, 240, 243, 244, 245, 247, 249, 258, 262, 263, 264, 265, 266, 267, 268, 269, 270, 271, 272, 273, 275, 278, 280, 281, 283, 285, 286, 291, 292, 293, 294, 295, 296, 297, 298, 299, 300, 305, 313, 323, 324, 325, 327, 328, 329, 330, 331, 332, 333, 335, 336, 396, or 428, wherein numbering of amino acid residues in said Fc domain is according to the EU index as in Kabat. In some embodiments, wherein the linker is covalently bound to a residue of the antibody construct selected from the group consisting of a lysine residue, cysteine residue, and a glutamine residue, or is covalently bound to said antibody construct using a Sortase A linker.

In some embodiments, a linker-immune-stimulatory compound (an ATAC) can be formed by conjugating a noncleavable maleimide-PEG4 linker containing a succinimide group with an immune-stimulatory compound. For example, an ATAC can be N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (ATAC11); N-(5-(2-amino-3-pentylquinolin-5-yl)pentyl)-1-(3-(2,5-dioxo-2,5-dihydro-1H-pyrrol-1-yl)propanamido)-3,6,9,12-tetraoxapentadecan-15-amide (ATAC12); 1-(3-(2,5-dioxo-2,5-dihydro-1H-pyrrol-1-yl)propanamido)-N-(3-pentylquinolin-2-yl)-3,6,9,12-tetraoxapentadecan-15-amide (ATAC13); 1-(3-(2,5-dioxo-2,5-dihydro-1H-pyrrol-1-yl)propanamido)-N-(1-isobutyl-1H-imidazo[4,5-c]quinolin-4-yl)-3,6,9,12-tetraoxapentadecan-15-amide (ATAC14); 1-(3-(2,5-dioxo-2,5-dihydro-1H-pyrrol-1-yl)propanamido)-N-methyl-N-(2-(3-(7-methylbenzo[1,2-d:3,4-d′]bis(thiazole)-2-yl)ureido)ethyl)-3,6,9,12-tetraoxapentadecan-15-amide (ATAC15); (S)-1-(3-(2,5-dioxo-2,5-dihydro-1H-pyrrol-1-yl)propanamido)-N-(1-((7-methylbenzo[1,2-d:3,4-d′]bis(thiazole)-2-yl)amino)-1-oxo-3-phenylpropan-2-yl)-3,6,9,12-tetraoxapentadecan-15-amide (ATAC16); N-(benzo[d]thiazol-2-yl)-1-(3-(2,5-dioxo-2,5-dihydro-1H-pyrrol-1-yl)propanamido)-N-((8-hydroxyquinolin-7-yl)(4-(trifluoromethoxy)phenyl)methyl)-3,6,9,12-tetraoxapentadecan-15-amide (ATAC17); N-((2R,3R,3aS,7aR,9R,10R,10aS,14aR)-2,9-bis(2-amino-6-oxo-1H-purin-9(6H)-yl)-5,10,12-trihydroxy-5,12-dioxidodecahydrodifuro[3,2-d:3′,2′-j][1,3,7,9,2,8]tetra-oxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC18); N-((2R,3R,3aS,7aR,9R,10R,10aS,14aR)-2,9-bis(2-amino-6-oxo-1H-purin-9(6H)-yl)-10-hydroxy-5,12-dimercapto-5,12-dioxidodecahydrodifuro[3,2-d:3′,2′-j][1,3,7,9,2,8]tetraoxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC19); N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetra-oxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC20); or N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetraoxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC21).

An ATAC can be formed by conjugating a cleavable linker containing a valine-alanine or valine-citrulline dipeptide, a PABA group and a maleimide group with an immune-stimulatory compound. For example, an ATAC can be 4-((S)-2-((S)-2-(6-(2,5-dioxo-2,5-dihydro-1H-pyrrol-1-yl)hexanamido)-3-methylbutanamido)propanamido)benzyl ((4-amino-1-(2-hydroxy-2-methyl-propyl)-1H-imidazo[4,5-c]quinolin-2-yl)methyl)(ethyl)carbamate (ATAC22); 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate (ATAC23); 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate (ATAC24); 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt (ATAC25); 2-(3-{2-[N-Methyl({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino]ethyl}ureido)-7-methyl-1,6-dithia-3,8-diaza-as-indacene (ATAC26); 2-{[(8-Hydroxy-7-quinolyl)(p-trifluoromethoxyphenyl)methyl]({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino}-1,3-benzothiazole (ATAC27); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC28); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}propionylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC29); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-9-hydroxy-3,12-dimercapto-2.4.7.11.13.16-hexaoxa-3λ5.12λ5-diphosphatricyclo[13.3.0.06,10]octadecane-3,12-dione (ATAC30); {p-[(S)-2-{(S)-2-[6-(2,5-Dioxo-1H-pyrrol-1-yl)hexanoylamino]-3-methylbutyrylamino}-5-ureidovalerylamino]phenyl}methyl 9-{(1S,6R,8R,9S,10S,15R,17R,18S)-8-(2-amino-6-oxo-1,9-dihydropurin-9-yl)-3,12-dihydroxy-9,18-dihydroxy-3,12-dioxo-2.4.7.11.13.16-hexaoxa-3λ5.12λ5-diphosphatricyclo[13.2.1.06,10]octadec-17-yl}-9a-adenineecarboxylate (ATAC31; 1-{6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexyl}-1H-pyrrole-2,5-dione (ATAC32); 1-{[4-({6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexylamino}carbonyl)cyclohexyl]methyl}-1H-pyrrole-2,5-dione (ATAC33); or 1-[(4-{[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-carbonyl}cyclohexyl)methyl]-1H-pyrrole-2,5-dione (ATAC34).

An ATAC can be formed by conjugating a noncleavable maleimide-PEG4 linker containing an activated ester such as a pentafluorophenyl group or an N-hydroxysuccinimide group with an immune-stimulatory compound. For example, an ATAC can be pentafluorophenyl 25-(2-amino-3-pentylquinolin-5-yl)-19-oxo-4,7,10,13,16-pentaoxa-20-azapentacosanoate (ATAC1); perfluorophenyl 3-((4-amino-1-(2-hydroxy-2-methylpropyl)-1H-imidazo[4,5-c]quinolin-2-yl)methyl)-4-oxo-7,10,13,16,19-pentaoxa-3-azadocosan-22-oate (ATAC2); pentafluorophenyl 25-(2-amino-3-pentylquinolin-5-yl)-19-oxo-4,7,10,13,16-pentaoxa-20-azapentacosanoate (ATAC3); or 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4).

An ATAC can be formed by conjugating a cleavable linker containing a valine-alanine or valine-citrulline dipeptide, a PABA group and an activated ester such as a pentafluorophenyl group or an N-hydroxysuccinimde group to an immune-stimulatory compound. For example, an ATAC can be 2,5-dioxopyrrolidin-1-yl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC5); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC6); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC7); perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC8); perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC9); or perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC10).

An antibody construct of a conjugate can comprise an anti-CD40 antibody. An anti-CD40 antibody can comprise two SBT-040-G1WT heavy chains and two light chains from a SBT-040 antibody, which can be referred to as SBT-040-WT. An anti-CD40 antibody can comprise two SBT-040-G1VLPLL heavy chains and two light chains from a SBT-040 antibody, which can be referred to as SBT-040-VLPLL. An anti-CD40 antibody can comprise two SBT-040-G1DE heavy chains and two light chains from a SBT-040 antibody, which can be referred to as SBT-040-DE. An anti-CD40 antibody can comprise two SBT-040-G1AAA heavy chains and two light chains from a SBT-040 antibody, which can be referred to as SBT-040-AAA. An anti-CD40 antibody can comprise two IgG2 heavy chains and two light chains from a SBT-040 antibody, which can be referred to as SBT-040-G2.

A conjugate can comprise SBT-040-WT-ATAC1. A conjugate can comprise SBT-040-WT-ATAC2. A conjugate can comprise SBT-040-WT-ATAC3. A conjugate can comprise SBT-040-WT-ATAC4. A conjugate can comprise SBT-040-WT-ATAC5. A conjugate can comprise SBT-040-WT-ATAC6. A conjugate can comprise SBT-040-WT-ATAC7. A conjugate can comprise SBT-040-WT-ATAC8. A conjugate can comprise SBT-040-WT-ATAC9. A conjugate can comprise SBT-040-WT-ATAC10. A conjugate can comprise SBT-040-WT-ATAC11. A conjugate can comprise SBT-040-WT-ATAC12. A conjugate can comprise SBT-040-WT-ATAC13. A conjugate can comprise SBT-040-WT-ATAC14. A conjugate can comprise SBT-040-WT-ATAC15. A conjugate can comprise SBT-040-WT-ATAC16. A conjugate can comprise SBT-040-WT-ATAC17. A conjugate can comprise SBT-040-WT-ATAC18. A conjugate can comprise SBT-040-WT-ATAC19. A conjugate can comprise SBT-040-WT-ATAC20. A conjugate can comprise SBT-040-WT-ATAC21. A conjugate can comprise SBT-040-WT-ATAC22. A conjugate can comprise SBT-040-WT-ATAC23. A conjugate can comprise SBT-040-WT-ATAC24. A conjugate can comprise SBT-040-WT-ATAC25. A conjugate can comprise SBT-040-WT-ATAC26. A conjugate can comprise SBT-040-WT-ATAC27. A conjugate can comprise SBT-040-WT-ATAC28. A conjugate can comprise SBT-040-WT-ATAC29. A conjugate can comprise SBT-040-WT-ATAC30. A conjugate can comprise SBT-040-WT-ATAC31. A conjugate can comprise SBT-040-WT-ATAC32. A conjugate can comprise SBT-040-WT-ATAC33. A conjugate can comprise SBT-040-WT-ATAC34. A conjugate can comprise SBT-040-VLPLL-ATAC1. A conjugate can comprise SBT-040-VLPLL-ATAC2. A conjugate can comprise SBT-040-VLPLL-ATAC3. A conjugate can comprise SBT-040-VLPLL-ATAC4. A conjugate can comprise SBT-040-VLPLL-ATAC5. A conjugate can comprise SBT-040-VLPLL-ATAC6. A conjugate can comprise SBT-040-VLPLL-ATAC7. A conjugate can comprise SBT-040-VLPLL-ATAC8. A conjugate can comprise SBT-040-VLPLL-ATAC9. A conjugate can comprise SBT-040-VLPLL-ATAC10. A conjugate can comprise SBT-040-VLPLL-ATAC11. A conjugate can comprise SBT-040-VLPLL-ATAC12. A conjugate can comprise SBT-040-VLPLL-ATAC13. A conjugate can comprise SBT-040-VLPLL-ATAC14. A conjugate can comprise SBT-040-VLPLL-ATAC15. A conjugate can comprise SBT-040-VLPLL-ATAC16. A conjugate can comprise SBT-040-VLPLL-ATAC17. A conjugate can comprise SBT-040-VLPLL-ATAC18. A conjugate can comprise SBT-040-VLPLL-ATAC19. A conjugate can comprise SBT-040-VLPLL-ATAC20. A conjugate can comprise SBT-040-VLPLL-ATAC21. A conjugate can comprise SBT-040-VLPLL-ATAC22. A conjugate can comprise SBT-040-VLPLL-ATAC23. A conjugate can comprise SBT-040-VLPLL-ATAC24. A conjugate can comprise SBT-040-VLPLL-ATAC25. A conjugate can comprise SBT-040-VLPLL-ATAC26. A conjugate can comprise SBT-040-VLPLL-ATAC27. A conjugate can comprise SBT-040-VLPLL-ATAC28. A conjugate can comprise SBT-040-VLPLL-ATAC29. A conjugate can comprise SBT-040-VLPLL-ATAC30. A conjugate can comprise SBT-040-VLPLL-ATAC31. A conjugate can comprise SBT-040-VLPLL-ATAC32. A conjugate can comprise SBT-040-VLPLL-ATAC33. A conjugate can comprise SBT-040-VLPLL-ATAC34. A conjugate can comprise SBT-040-VLPLL-ATAC34. A conjugate can comprise SBT-040-DE-ATAC1. A conjugate can comprise SBT-040-DE-ATAC2. A conjugate can comprise SBT-040-DE-ATAC3. A conjugate can comprise SBT-040-DE-ATAC4. A conjugate can comprise SBT-040-DE-ATAC5. A conjugate can comprise SBT-040-DE-ATAC6. A conjugate can comprise SBT-040-DE-ATAC7. A conjugate can comprise SBT-040-DE-ATAC8. A conjugate can comprise SBT-040-DE-ATAC9. A conjugate can comprise SBT-040-DE-ATAC10. A conjugate can comprise SBT-040-DE-ATAC11. A conjugate can comprise SBT-040-DE-ATAC12. A conjugate can comprise SBT-040-DE-ATAC13. A conjugate can comprise SBT-040-DE-ATAC14. A conjugate can comprise SBT-040-DE-ATAC15. A conjugate can comprise SBT-040-DE-ATAC16. A conjugate can comprise SBT-040-DE-ATAC17. A conjugate can comprise SBT-040-DE-ATAC18. A conjugate can comprise SBT-040-DE-ATAC19. A conjugate can comprise SBT-040-DE-ATAC20. A conjugate can comprise SBT-040-DE-ATAC21. A conjugate can comprise SBT-040-DE-ATAC22. A conjugate can comprise SBT-040-DE-ATAC23. A conjugate can comprise SBT-040-DE-ATAC24. A conjugate can comprise SBT-040-DE-ATAC25. A conjugate can comprise SBT-040-DE-ATAC26. A conjugate can comprise SBT-040-DE-ATAC27. A conjugate can comprise SBT-040-DE-ATAC28. A conjugate can comprise SBT-040-DE-ATAC29. A conjugate can comprise SBT-040-DE-ATAC30. A conjugate can comprise SBT-040-DE-ATAC31. A conjugate can comprise SBT-040-DE-ATAC32. A conjugate can comprise SBT-040-DE-ATAC33. A conjugate can comprise SBT-040-DE-ATAC34. A conjugate can comprise SBT-040-AAA-ATAC1. A conjugate can comprise SBT-040-AAA-ATAC2. A conjugate can comprise SBT-040-AAA-ATAC3. A conjugate can comprise SBT-040-AAA-ATAC4. A conjugate can comprise SBT-040-AAA-ATAC5. A conjugate can comprise SBT-040-AAA-ATAC6. A conjugate can comprise SBT-040-AAA-ATAC7. A conjugate can comprise SBT-040-AAA-ATAC8. A conjugate can comprise SBT-040-AAA-ATAC9. A conjugate can comprise SBT-040-AAA-ATAC10. A conjugate can comprise SBT-040-AAA-ATAC11. A conjugate can comprise SBT-040-AAA-ATAC12. A conjugate can comprise SBT-040-AAA-ATAC13. A conjugate can comprise SBT-040-AAA-ATAC14. A conjugate can comprise SBT-040-AAA-ATAC15. A conjugate can comprise SBT-040-AAA-ATAC16. A conjugate can comprise SBT-040-AAA-ATAC17. A conjugate can comprise SBT-040-AAA-ATAC18. A conjugate can comprise SBT-040-AAA-ATAC19. A conjugate can comprise SBT-040-AAA-ATAC20. A conjugate can comprise SBT-040-AAA-ATAC21. A conjugate can comprise SBT-040-AAA-ATAC22. A conjugate can comprise SBT-040-AAA-ATAC23. A conjugate can comprise SBT-040-AAA-ATAC24. A conjugate can comprise SBT-040-AAA-ATAC25. A conjugate can comprise SBT-040-AAA-ATAC26. A conjugate can comprise SBT-040-AAA-ATAC27. A conjugate can comprise SBT-040-AAA-ATAC28. A conjugate can comprise SBT-040-AAA-ATAC29. A conjugate can comprise SBT-040-AAA-ATAC30. A conjugate can comprise SBT-040-AAA-ATAC31. A conjugate can comprise SBT-040-AAA-ATAC32. A conjugate can comprise SBT-040-AAA-ATAC33. A conjugate can comprise SBT-040-AAA-ATAC34. The Kd for binding of the CD40 binding domain of any of these conjugates to CD40 can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the CD40 binding domain to CD40 in the absence of the immune-stimulatory compound or ATAC. The Kd for binding of the CD40 binding domain of any of these conjugates to CD40 can be less than 10 nM. The Kd for binding of the CD40 binding domain of any of the conjugates to CD40 can be less than 100 nM, less than 50 nM, less than 20 nM, less than 5 nM, less than 1 nM, or less than 0.1 nM. The Kd for binding of the Fc domain of any of the conjugates to an Fc receptor can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the Fc domain to the Fc receptor in the absence of the immune-stimulatory compound or ATAC. The Kd for binding of the Fc domain of any of the conjugates to an Fc receptor can be less than 10 nM. The Kd for binding of the Fc domain of any of the conjugates to an Fc receptor can be less than 100 nM, less than 50 nM, less than 20 nM, less than 5 nM, less than 1 nM, or less than 0.1 nM.

In a conjugate, an antibody can be linked to an immune-stimulatory compound in such a way that the antibody can still bind to an antigen and the Fc domain of the antibody can still bind to an FcR or FcR-mediated signaling resulting from the Fc domain of the antibody from binding to an FcR. In a conjugate, an antibody construct is linked to an immune-stimulatory compound in such a way that the linking does not interfere with ability of the antigen binding domain of the antibody construct to bind to antigen, the ability of the Fc domain of the antibody construct to bind to an FcR, or FcR-mediated signaling resulting from the Fc domain of the antibody construct from binding to an FcR. In a conjugate, an immune-stimulatory compound can be linked to an antibody construct in such a way the linking does not interfere with the ability of the immune-stimulatory compound to bind to its receptor. A conjugate can produce stronger immune stimulation and a greater therapeutic window than components of the conjugate alone. In an anti-CD40 antibody linked to a TLR agonist conjugate, the combination of CD40 agonism, TLR agonism, and an accessible Fc domain of the anti-CD40 antibody to allow FcR-mediated signaling can produce stronger immune stimulation and a greater therapeutic window than the CD40 agonism, TLR agonism, or the FcR-mediated signaling alone.

In some embodiments, the recombinant bispecific antibody further comprises an immune stimulatory compound and a linker, wherein the linker links the immune-stimulatory compound to the Fc comprising domain.

In some aspects, a method of making a conjugate comprises linking an antibody construct as described herein to an immune stimulatory compound by a linker.

### Synthesis of Immune-Stimulatory Compounds

An immune stimulatory compound can be synthesized as shown in Scheme A1.

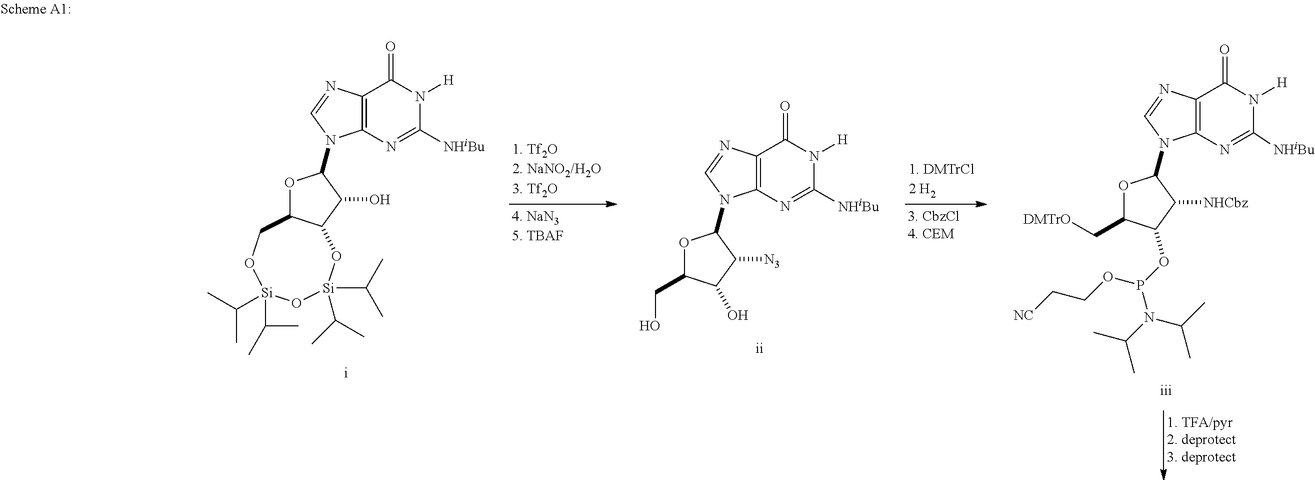

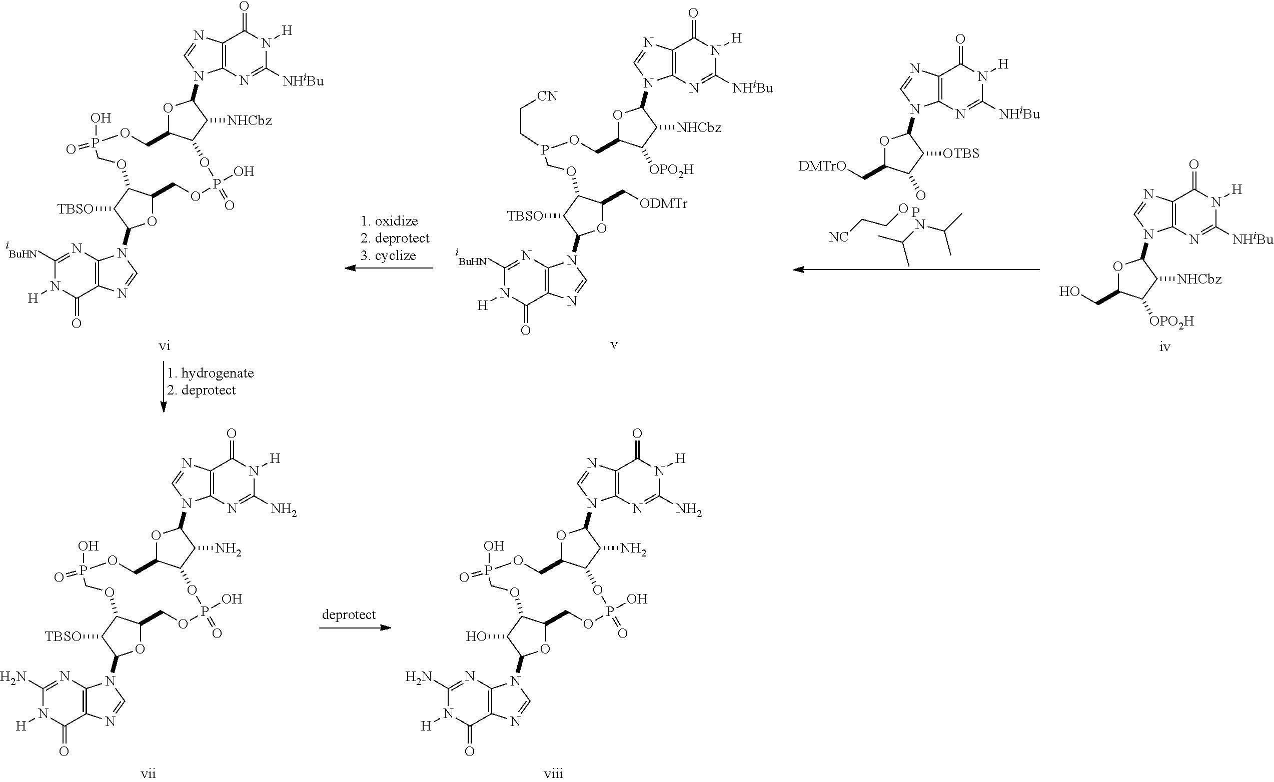

Synthesis of the C-2′ amino cyclic dinucleotide (viii) can be accomplished using a multistep synthesis as outlined in scheme A1 above.

### Synthesis of Exemplary ATAC Compounds

An ATAC compound can be synthesized by various methods. For example, ATAC compounds, such as ATAC1-ATAC4, can be synthesized as shown in Scheme B1.

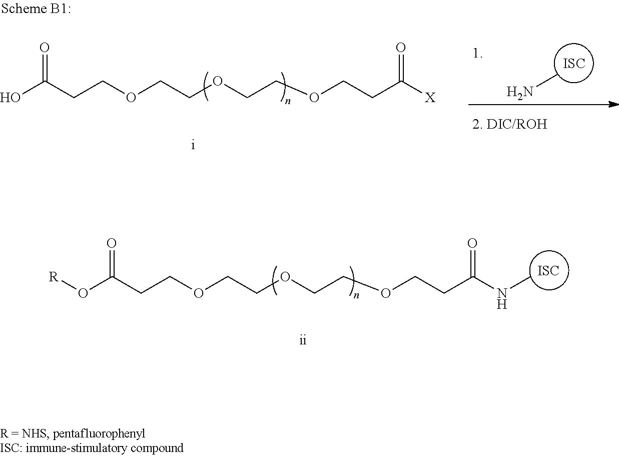

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An ATAC compound can be synthesized by various methods. For example, ATAC compounds, such as ATAC5-ATAC10, can be synthesized as shown in Scheme B2.

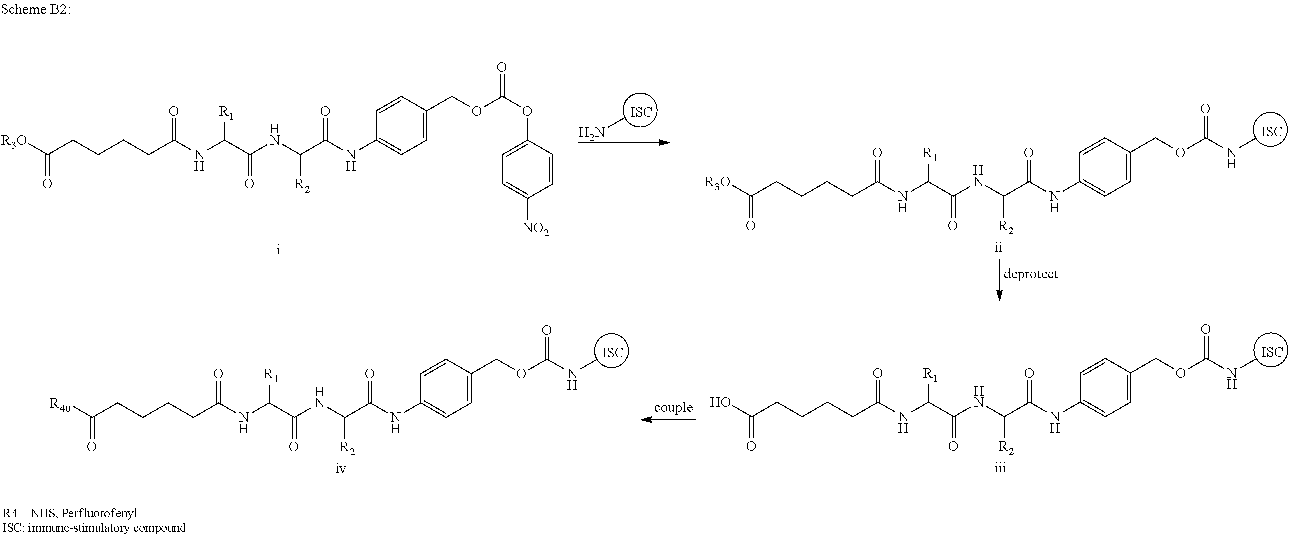

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

An ATAC compound can be synthesized by various methods. For example, ATAC compounds, such as ATAC11-ATAC21, can be synthesized as shown in Scheme B3.

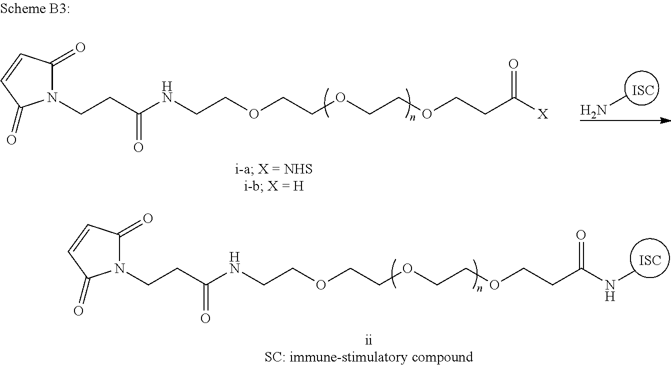

An activated carboxylic ester such as (i-a) can be reacted with an appropriately substituted amine containing immune-stimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired ATAC compounds.

An ATAC compound can be synthesized by various methods. For example, ATAC compounds, such as ATAC22-ATAC31, can be synthesized as shown in Scheme B4.

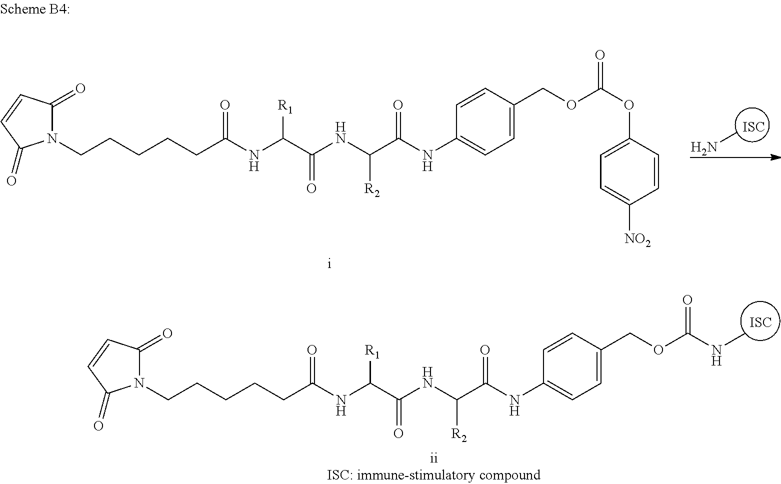

An activated carbonate such as (i) can be reacted with an appropriately substituted amine containing immune-stimulatory compound to afford carbamates (ii) as the target ATAC compounds.

An ATAC compound can be synthesized by various methods. For example, ATAC compounds, such as ATAC32-ATAC34, can be synthesized as shown in Scheme B5.

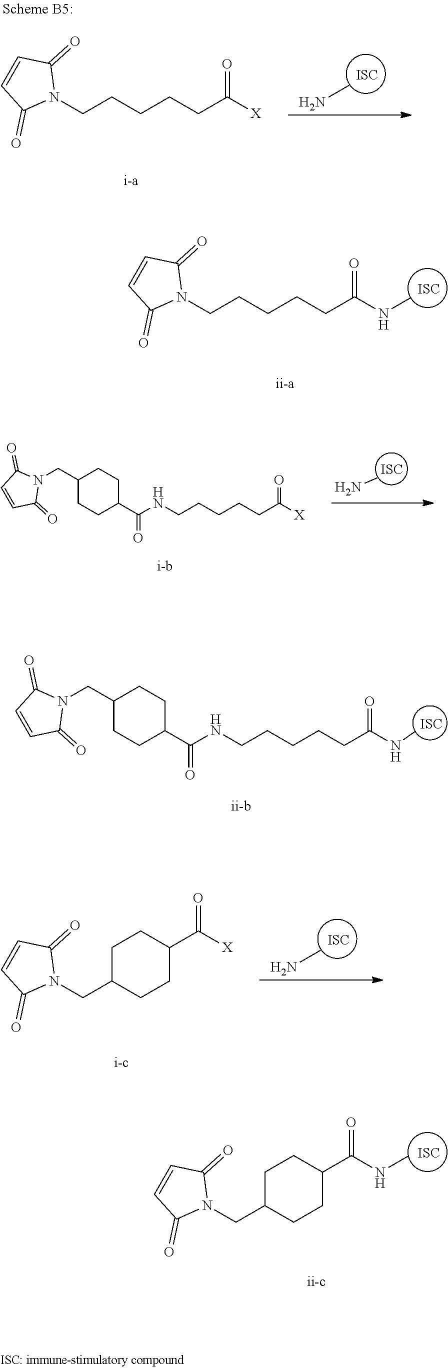

An activated carboxylic acid such as (i-a, i-b, i-c) can be reacted with an appropriately substituted amine containing immune-stimulatory compound to afford amides (ii-a, ii-b, ii-c) as the target ATAC compounds.

### Synthesis of Exemplary TLR8 Agonist Compounds

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

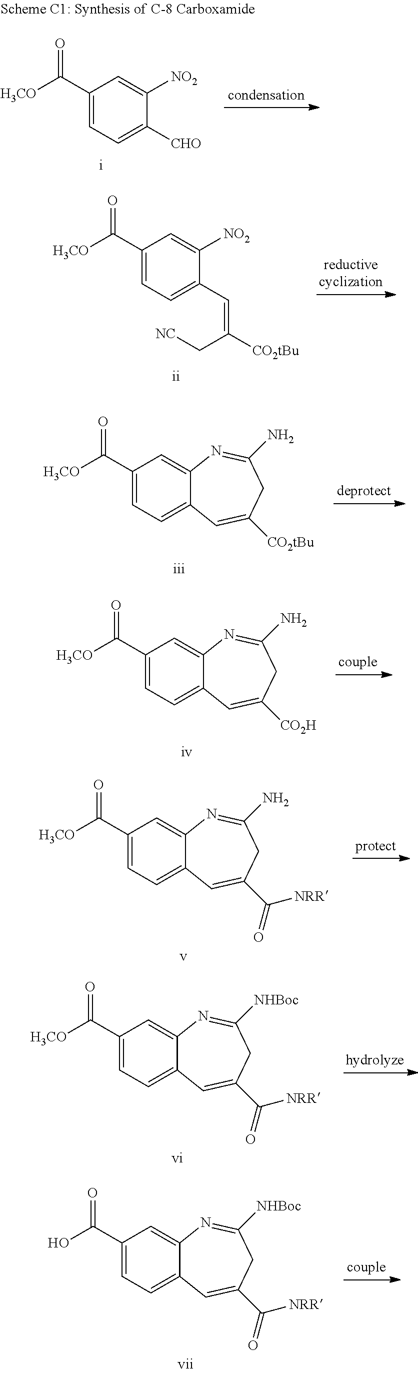

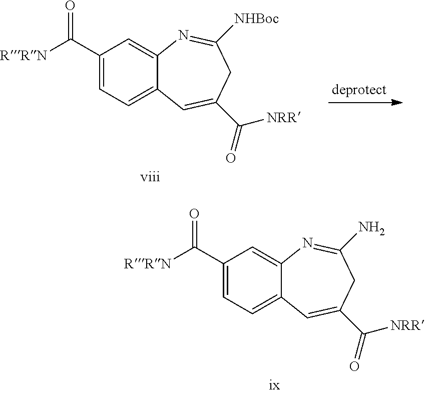

React an aldehyde (i) with an appropriately Wittig reagent, such as tert-butyl 3-cyano-2-(triphenylphosphorylidene)propanoate, at elevated temperatures to afford an olefin (ii), which undergoes reductive cyclization by treating the olefin (ii) with a reducing agent, such as iron powder in hot acetic acid, to afford azepines (iii). Deprotect the C-4 ester group by using a strong acid such as HCl to give compounds (iv), which is in turn coupled with a substituted amine using a coupling agent, such as BOP reagent. Protect the 2-amino substituent of compounds (v) with a tert-butoxycarbonyl group. Hydrolyze the resulting compounds (vi) with reagents such as LiOH in a mixture of THF and methanol to afford compounds (vii). Convert the C-8 carboxylic acid of (vii) to the amide group using known reagents such as HBTU and a tertiary amine base. Acid-mediated deprotection of compounds (viii) using a reagent such as TFA in dichloromethane provides the target compounds (ix).

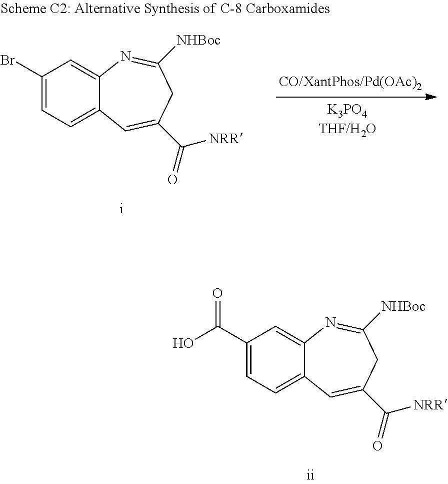

React (i) under standard conditions used for the carbonylation of aryl halides such as carbon monoxide, a palladium catalyst such as Pd(OAc)₂ and a ligand such as 4,5-bis(diphenylphosphino)-9,9-dimethylxanthene (XantPhos) and a base such as potassium phosphate in a mixture of THF and water to provide carboxylic acids (ii). Conversion to final products can then be carried out in a manner similar to that described in Scheme 1 (vii→ix).

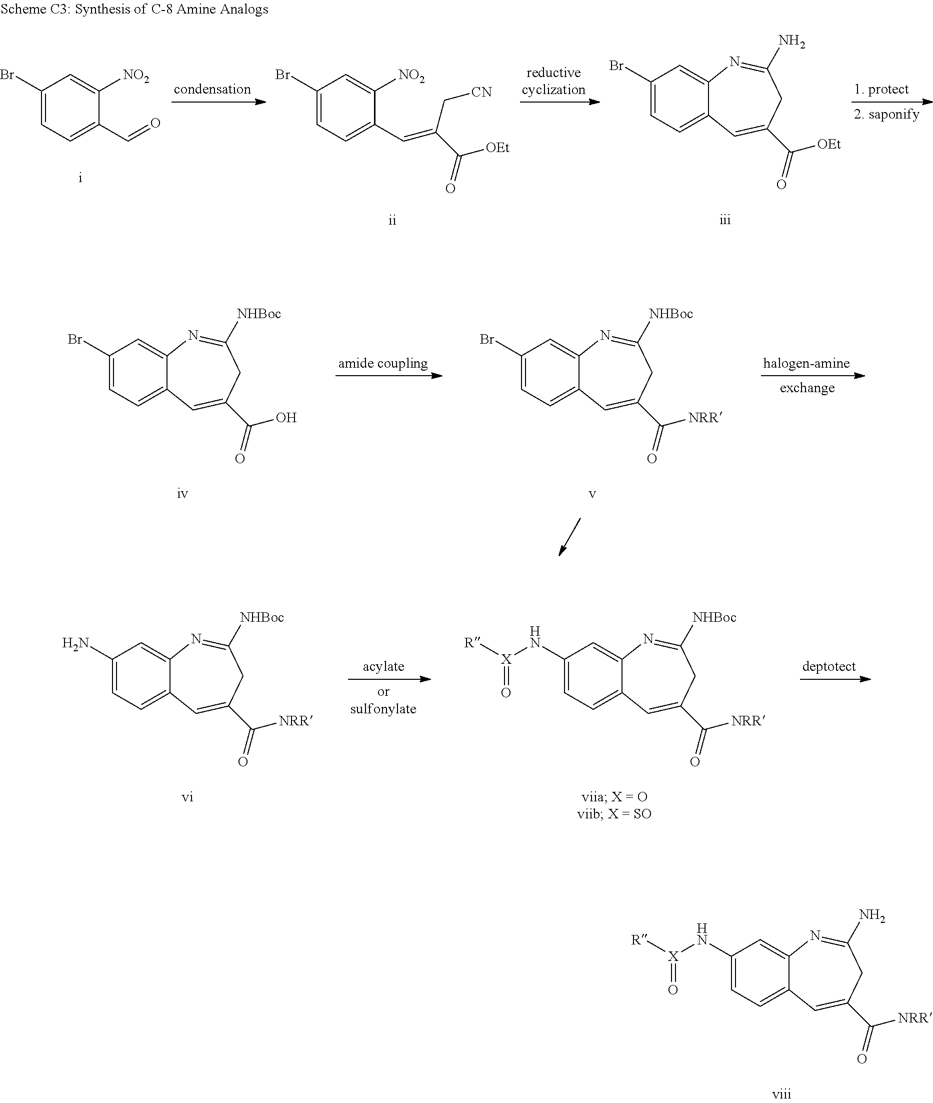

React an aldehyde (i) with an appropriately Wittig reagent, such as ethyl 3-cyano-2-(triphenylphosphorylidene)propanoate, at ambient temperature to afford an olefin (ii), which undergoes reductive cyclization by treating the olefin (ii) with a reducing agent, such as iron powder in hot acetic acid, to afford azepines (iii). Protect the C-2 amine group by using Boc anhydride to give compounds (iii), which is in turn saponified with an alkaline metal hydroxide such as LiOH to afford the carboxylic acid which is coupled with a substituted amine using a coupling agent, such as BOP reagent to provide compounds (iv). Convert the C-8 carboxylic acid of (v) to the amide group using known reagents such as EDCI/HOBT and a tertiary amine base. Halogen-amine exchange can be effected using standard methodology such as copper-mediated or palladium-catalyzed couplings (benzophenone imine/Pd(II)) to provide C-8 anilines (vi). Functionalization of amines (vi) by acylation or sulfonylation provides anilides (X═C) or sulfonamides (X═SO) compounds (vii). Alternatively, compounds (vii) can be prepared directly through a palladium-mediated coupling of bromide (v) and an appropriately substituted amide or sulfonamide. Acid-mediated deprotection of compounds (vii) using a reagent such as TFA in dichloromethane provides the target compounds (viii).

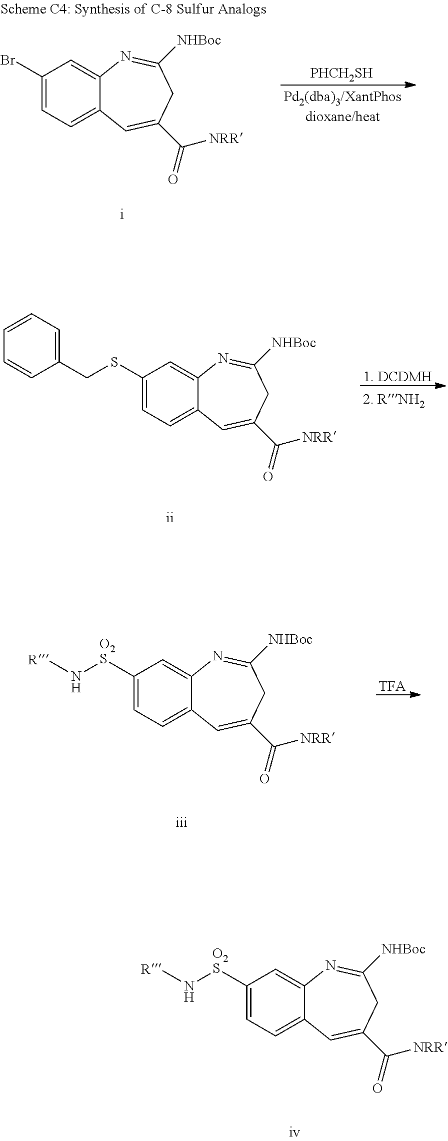

React (i) with benzyl thiol in the presence of a palladium catalyst such as Pd₂(dba)₃ and a ligand such as XantPhos at elevated temperatures to provide C-8 sulfides (ii). Oxidative chlorination of sulfides (ii) with a reagent such as 1,3-dichloro-5,5-dimethylhydantoin (DCDMH) affords intermediate sulfonyl chlorides which can be reacted with an appropriately substituted amine of structure R′″NH₂ to provides sulfonamides (iii). Acid-mediated deprotection of compounds (iii) using a reagent such as TFA in dichloromethane provides the target compounds (iv).

These conjugates can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

In some aspects, a conjugate comprises: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the conjugate when bound to the tumor antigen as measured by a cytokine release assay is greater than immune cell activation in the absence of binding to the tumor antigen.

In some aspects, a conjugate comprises: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein antigen presenting cells are conditionally activated when the conjugate is bound to the tumor antigen as measured by a cytokine release assay.

In some aspects, a conjugate for use in inducing immune cell activation comprising: a) an antibody construct comprising: i) first binding domain, wherein the first binding domain specifically binds to a tumor antigen; ii) a second binding domain, wherein the second binding domain specifically binds to an antigen on an antigen presenting cell, wherein the antigen is a molecule on the antigen presenting cell; and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein immune cell activation caused by the conjugate when bound to the tumor antigen as measured by a cytokine release assay is greater than immune cell activation in the absence of binding to the tumor antigen.

In some aspects, a conjugate for use in conditionally activating an antigen presenting cell comprising: a) an antibody construct comprising: i) first binding domain, wherein the first binding domain specifically binds to a tumor antigen; ii) a second binding domain, wherein the second binding domain specifically binds to an antigen on the antigen presenting cell, and iii) an Fc domain; b) an immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; wherein the first binding domain is attached to the Fc domain and the second binding domain is attached to the Fc domain or to a C-terminal end of a light chain of the first binding domain; wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and wherein antigen presenting cells are conditionally activated when the conjugate is bound to the tumor antigen as measured cytokine release assay.

In some aspects, a conjugate comprises: a) an immune-stimulatory compound; b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, 41BBL, MARCO, CLEC5A, Dectin 1, Dectin 2, CD64, CD32A, CD16A, HVEM, and CD32B, and a fragment thereof; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate comprises: a) an immune-stimulatory compound; b) an antibody construct comprising a first binding domain and an Fc domain, wherein: i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, OX40L, 41BBL, MARCO, CLEC5A, Dectin 1, Dectin 2, CD64, CD32A, CD16A, HVEM, CD32B, and CD47, and a fragment thereof, ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate comprises: a) an immune-stimulatory compound; b) an antibody construct comprising a first binding domain and an Fc domain, wherein: i) the first binding domain comprises a variable region comprising a set of CDR sequences that comprises at least 80% sequence identity to a set of variable region CDR sequences set forth in TABLE 3 or TABLE 11; ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

In some aspects, a conjugate for use in activating an immune cell comprises: a) an immune-stimulatory compound; b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, 41BBL, MARCO, CLEC5A, Dectin 1, Dectin 2, CD64, CD32A, CD16A, HVEM, and CD32B, and a fragment thereof; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprises: a) an immune-stimulatory compound; b) an antibody construct comprising a first binding domain and an Fc domain, wherein: i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, OX40L, 41BBL, MARCO, CLEC5A, Dectin 1, Dectin 2, CD64, CD32A, CD16A, HVEM, CD32B, and CD47, and a fragment thereof, ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprises: a) an immune-stimulatory compound; b) an antibody construct comprising a first binding domain and an Fc domain, wherein the first binding domain comprises a variable region comprising a set of CDR sequences that comprises at least 80% sequence identity to a set of variable region CDR sequences set forth in TABLE 3 or TABLE 11; c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen.

In some aspects, a conjugate for use in activating an immune cell comprising: a) an immune-stimulatory compound; b) an antibody construct comprising a first binding domain and an Fc domain, wherein: i) the first binding domain comprises a variable region comprising a set of CDR sequences that comprises at least 80% sequence identity to a set of variable region CDR sequences set forth in TABLE 3 or TABLE 11; ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and wherein immune cell activation caused by the conjugate when bound to the tumor antigen as measured by a cytokine release assay is greater than immune cell activation is greater than immune cell activation in the absence of binding to the tumor antigen. In some embodiments, the first binding domain comprises a variable region comprising V_{H} and V_{L} sequences at least 80% sequence identity to a pair of V_{H} and V_{L} sequences set forth in TABLE 5 or TABLE 13.

### Pharmaceutical Formulations

The conjugates, antibody constructs, recombinant bispecific antibodies and methods described herein can be considered useful as pharmaceutical compositions for administration to a subject in need thereof. As used herein the terms “individual,” “subject,” and “patient” are used interchangeably and include humans diagnosed with or suspected of being afflicted with a tumor, a cancer or other neoplasm. As will be appreciated by the skilled artisan, the following description of pharmaceutical formulations of conjugates and antibody constructs is applicable to recombinant bispecific antibodies and conjugates comprising recombinant bispecific antibodies.

Pharmaceutical compositions can comprise at least the antibody constructs and/or conjugates described herein and one or more pharmaceutically acceptable carriers, diluents, excipients, stabilizers, dispersing agents, suspending agents, and/or thickening agents. The pharmaceutical composition can comprise the antibody construct. The pharmaceutical composition can comprise the conjugate comprising an antibody construct and an immune-stimulatory compound, such as an agonist. The pharmaceutical composition can comprise the conjugate comprising an Fc domain, a binding domain, and an immune-stimulatory compound, such as an agonist. The pharmaceutical composition can comprise any conjugate described herein. The pharmaceutical composition can contain a anti-CD40 antibody. A pharmaceutical composition can comprise an anti-CD40 antibody conjugated to PAMP molecule. A pharmaceutical composition can comprise an anti-CD40 antibody conjugated to a DAMP molecule. A pharmaceutical composition can further comprise buffers, antibiotics, steroids, carbohydrates, drugs (e.g., chemotherapy drugs), radiation, polypeptides, chelators, adjuvants and/or preservatives.

In a pharmaceutical composition, the conjugates can have an average drug loading. The drug loading, p, is the average number of immune-stimulatory compound-linker molecules per antibody construct, or the number of immune-stimulatory compounds per antibody construct. P can range ranges from 1 to 20, or 1-100. In some conjugates, p is preferably from 1 to 8. The average number of immune-stimulatory compounds per antibody construct in a preparation may be characterized by conventional means such as mass spectroscopy, HIC, ELISA assay, and HPLC.

Pharmaceutical compositions can be formulated using one or more physiologically-acceptable carriers comprising excipients and auxiliaries. Formulation can be modified depending upon the route of administration chosen. Pharmaceutical compositions comprising a conjugate as described herein can be manufactured, for example, by lyophilizing the conjugate, mixing, dissolving, emulsifying, encapsulating or entrapping the conjugate. Pharmaceutical compositions comprising an antibody construct as described herein can be manufactured, for example, by lyophilizing the antibody construct, mixing, dissolving, emulsifying, encapsulating or entrapping the antibody construct. The pharmaceutical compositions can also include the conjugates or antibody constructs described herein in a free-base form or pharmaceutically-acceptable salt form.

Methods for formulation of the pharmaceutical compositions can include formulating any of the conjugates or antibody constructs described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use

Pharmaceutical compositions of the conjugates or antibody constructs described herein can comprise at least a conjugate or antibody construct as an active ingredient, respectively. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the pharmaceutical composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the pharmaceutical compositions described herein can be lyophilized or in powder form for reconstitution with a suitable vehicle, e.g., sterile pyrogen-free water, before use.

For parenteral administration, the recombinant antibodies and conjugates can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate or antibody construct with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In certain embodiments, the recombinant antibodies and conjugates of the current disclosure are administered in or packaged with a pharmaceutically acceptable carrier. The antibodies can be administered suspended in a sterile solution. In certain embodiments, the solution comprises 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, or dextran 40; amino acids, for example, glycine or arginine; antioxidants, for example, ascorbic acid, methionine; and chelating agents, for example, EGTA or EGTA. In certain embodiments, the antibodies and conjugates of the current disclosure are shipped/stored lyophilized and reconstituted before administration. In certain embodiments, lyophilized antibody formulations comprise a bulking agent such as mannitol, sorbitol, sucrose, trehalose, or dextran 40.

### Therapeutic Applications

The conjugates, antibody constructs, recombinant bispecific antibodies and pharmaceutical compositions thereof, and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. As will be appreciated by the skilled artisan, the following description of therapeutic applications of antibody constructs and conjugates comprising antibody constructs is applicable to recombinant bispecific antibodies and conjugates comprising recombinant bispecific antibodies.

The conjugates, antibody constructs, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the conjugates, antibody constructs, or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

Prevent, preventing and the like also can refer to the prevention of relapse of the disease or condition, e.g., tumor formation, in the patient. For example, an individual at risk of relapse of a tumor or other form of cancer after treatment and obtaining a state of remission can be treated with the methods of the present disclosure to present relapse.

A therapeutically effective amount can be the amount of conjugates, antibody constructs, or pharmaceutical compositions or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates, antibody constructs, or pharmaceutical compositions described herein that can be used in therapy can be formulated and dosages established in a fashion consistent with good medical practice taking into account the disorder to be treated, the condition of the individual patient, the site of delivery of the conjugate, antibody construct, or pharmaceutical composition, the method of administration and other factors known to practitioners. The conjugates, antibody constructs, or pharmaceutical compositions can be prepared according to the description of preparation described herein.

One of ordinary skill in the art would understand that the amount, duration and frequency of administration of a pharmaceutical composition, antibody construct, or conjugate described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, conjugates, antibody constructs, and pharmaceutical compositions described herein can be for administration to a subject in need thereof. Often, administration of the conjugates, recombinant antibodies, antibody constructs, or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition, antibody construct, or conjugate can be administered to a subject by additional routes of administration, for example, by inhalation, oral, dermal, intranasal, or intrathecal administration.

Pharmaceutical compositions, antibody constructs, or conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The conjugates, antibody constructs, or pharmaceutical compositions can be administered to the subject in need thereof in cycles of 21 days, 14 days, 10 days, 7 days, 4 days or daily over a period of one to seven days.

In some aspects, a pharmaceutical composition comprises any recombinant bispecific antibody as described herein and a pharmaceutically acceptable carrier.

In some aspects, a pharmaceutical composition comprises the conjugate or antibody construct of as described herein and a pharmaceutically acceptable carrier.

### Increased Dosages and Reduced Side-Effects

In certain embodiments, the conjugates described herein can be administered in a dosage that is about 10%, about 25%, about 50%, about 100% or greater than an antibody from which one of the tumor antigen binding domains or binding domains that bind to a molecule on a target cell, such as a tumor antigen, or on an immune cell, such as an antigen presenting cell (APC), is derived. For example, a common regimen for administering pertuzumab comprises 840 mg intravenous (IV) administered as an initial dose over 60 minutes, followed every 3 weeks thereafter by 420 mg IV over 30 to 60 minutes. Using a conjugate of this disclosure, an initial dosage can range from 900 mg to 1700 mg or more and a maintenance dose can range from 450 mg to 900 mg or more. An increased initial dose and/or maintenance dose can be used with an conjugate of this disclosure, such as a bispecific tumor targeting conjugate comprising a tumor antigen binding domain that binds to a tumor antigen with at least 80% homology to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, or a fragment thereof, as compared with the respective parental monoclonal antibody. An increased initial dose and/or maintenance dose also can be used with any bispecific tumor targeting conjugate of this disclosure, such as bispecific tumor targeting conjugate comprising a tumor antigen binding domain that binds to a tumor antigen with at least 80% homology to the amino acid sequence of LRRC15, GLP-3, CLDN6, CLDN16, UPK1B, VTCN1 and STRA6, or a fragment thereof, as compared with the respective parental monoclonal antibody. Antibodies that bind costimulatory molecules or other cell surface molecules on immune cells such as APCs can have small therapeutic windows and high dose-limiting toxicity. For example, the antibody CP-870,893 can be shown to have a maximum tolerated dosage of 0.2 mg/kg to 0.3 mg/kg. Using a bispecific tumor targeting conjugate of this disclosure can allow administration of the conjugate at greater than 0.2 mg/kg to 0.3 mg/kg that comprises a binding domain derived from CP-870,893 or any binding domain that binds to a molecule on an immune cell with at least 80% homology to the amino acid sequence of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, CD47, or a fragment thereof. Using a bispecific tumor targeting conjugate of this disclosure can allow administration of the conjugate at greater than 0.2 mg/kg to 0.3 mg/kg that comprises a binding domain derived from CP-870,893 or any binding domain that binds to a molecule on an immune cell with at least 80% homology to the amino acid sequence of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, CD47, TNFR2, TREM2, or a fragment thereof. Using a bispecific tumor targeting conjugate of this disclosure can allow administration of the conjugate at greater than 0.2 mg/kg to 0.3 mg/kg that comprises a binding domain derived from CP-870,893 or any binding domain that binds to a molecule on an APC with at least 80% homology to the amino acid sequence of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, CD47, or a fragment thereof. Using a bispecific tumor targeting conjugate of this disclosure can allow administration of the conjugate at greater than 0.2 mg/kg to 0.3 mg/kg that comprises a binding domain derived from CP-870,893 or any binding domain that binds to a molecule on an APC with at least 80% homology to the amino acid sequence of CD40, DEC-205, CD36 mannose scavenger receptor 1, DC-SIGN, CLEC9A, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, CD47, TNFR2, TREM2, or a fragment thereof. In certain embodiments, using a bispecific tumor targeting conjugate of this disclosure can allow administration of a conjugate that comprises a binding domain that binds to a molecule on an immune cell, such as an antigen presenting cell, at greater levels than an antibody comprising a binding domain that binds to that molecule on an antigen presenting cell. In certain embodiments, a bispecific tumor targeting conjugate that comprises a binding domain that binds to a molecule on an immune cell, such as an antigen presenting cell, can be administered at levels equivalent to that of an antibody from which the binding domain is derived. In certain embodiments, the bispecific tumor targeting conjugate can be administered at a level higher than the maximum tolerated dose for bispecific tumor targeting antibody construct in the absence of a conjugated immune-stimulatory compound. In certain embodiments, if the bispecific tumor targeting antibody construct is conjugated to a chemotherapeutic agent, then the conjugate can be administered at a level higher than the maximum tolerated dose for that chemotherapeutic agent administered in the absence of the chemotherapeutic agent conjugated to a bispecific tumor targeting antibody construct. In certain embodiments, administration of the bispecific tumor targeting conjugate can be associated with fewer side effects than an antibody from which one of binding domains is derived. In certain embodiments, administration of the bispecific tumor targeting conjugate can be associated with fewer side effects than when the immune-stimulatory compound is conjugated to an antibody from which one of binding domains is derived. In certain embodiments, administration of the bispecific tumor targeting antibody construct conjugated to a chemotherapeutic agent can be associated with fewer side effects than when the chemotherapeutic agent is conjugated to an antibody from which one of binding domains is derived.

In some embodiments described herein is a method of treating a subject in need thereof, comprising administering to the individual a therapeutic dose of a recombinant bispecific antibody, or the pharmaceutical composition, to an individual having cancer. In some embodiments, the administering comprises administering the recombinant bispecific antibody, or the pharmaceutical composition, intravenously, cutaneously, subcutaneously, or injected at a site of affliction. In some embodiments, the recombinant bispecific antibody induces more potent immune activation to cancer cells or tissue as compared to non-cancerous tissue. In some further embodiments, when the recombinant antibody is administered intraveneously to the subject at the minimum anticipated biological effect level of the recombinant antibody, a biological effect of the recombinant antibody is greater when the recombinant antibody is bound to the tumor associated antigen at the cancer and to the molecule on the antigen presenting cell as compared to the biological effect of the recombinant antibody when it is not bound to the tumor associated antigen but is bound to the molecule on the antigen presenting cell.

In additional aspects, the recombinant bispecific antibodies and conjugates thereof can have an increased minimum anticipated biological effect level (MABEL), as compared to a corresponding mono-specific antibody or conjugate. The MABEL of the recombinant bispecific antibody is higher than the MABEL of the control monospecific antibody or conjugate. When the recombinant bispecific antibody is administered to a subject at the MABEL, the biological effect is greater when the target antigen binding domain is bound to its tumor associated antigen as compared to the biological effect of the recombinant antibody when the recombinant antibody is not bound to the tumor associated antigen. In some embodiments, the biological effect is greater when the target antigen binding domain is bound to its tumor associated antigen and the effector antigen binding domain is bound to the molecule on the antigen presenting cell as compared to the biological effect of the recombinant antibody when the recombinant antibody is not bound to the tumor associated antigen but the effector antigen binding domain is bound to the molecule on the antigen presenting cell. The biological effect can be immune activation.

MABEL is the anticipated dose needed that results in a biological effect in a human subject, in which a biological effect is measured by an in vitro, ex vivo, and/or in vivo assay that measures a selected biological, biochemical, pharmacological, or pharmacodynamic effect. A selected biological, biochemical, pharmacological, or pharmacodynamic effect can be secretion of one or more cytokines, secretion of one or more chemokines, expression level of one or more cell surface proteins associated with immune stimulation, or activity of one or more immune cell functions. For example, the secretion of one or more cytokines can be the secretion of one or more of IL-6, TNFα, IL-12p40, IL-12p70, and/or IL-10. Secretion of one or more chemokines can be IL-8, IP-10, MIP-1α, and/or MIP-113. Expression level of one or more cell surface proteins associated with immune stimulation can be CD54, CD86, CD80, MHC class II, and/or CD83. Activity of one or more immune cell functions can be antibody-dependent cell-mediated cytotoxicity, antibody dependent cellular phagocytosis, and/or antigen cross-presentation.

In some aspects, when dosing intraveneously at the MABEL of the recombinant antibody, when the recombinant antibody is bound to the tumor associated antigen the biological effect can be at least two times, five times, or ten times greater than the biological effect of the recombinant antibody when the recombinant antibody is not bound to the tumor associated antigen. The biological effect can be immune activation.

In some aspects, method of treating a subject in need thereof, comprising administering to the subject a therapeutic dose of any recombinant bispecific antibody as described herein or the pharmaceutical composition of any recombinant bispecific antibody as described herein. In some embodiments, the subject has cancer.

In some embodiments, the recombinant bispecific antibody or the pharmaceutical composition is administered intravenously, cutaneously, subcutaneously, or injected at a site of affliction. In some embodiments, the recombinant bispecific antibody induces greater immune activation against a cancer as measured by a decrease in cancer cell number or volume as compared to non-cancerous tissue.

In some embodiments, the recombinant bispecific antibody is administered intravenously to the subject at a minimum anticipated biological effect level of the recombinant bispecific antibody, a biological effect of the recombinant bispecific antibody is greater when the recombinant bispecific antibody is bound to the tumor associated antigen and to the antigen on the antigen presenting cell as compared to the biological effect of the recombinant bispecific antibody when it is not bound to the tumor associated antigen but is bound to the antigen on the antigen presenting cell; and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation.

In some embodiments, the recombinant bispecific antibody is administered intravenously to the subject at the minimum anticipated biological effect level of the recombinant bispecific antibody, it induces a greater biological effect at the site of the cancer than at a non-cancerous site and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation.

### Additional Therapeutic Agents

In certain embodiments, conjugates, antibody constructs, recombinant bispecific antibodies, pharmaceutical compositions thereof, and methods provided herein can be administered with or during treatment with an additional therapeutic agent. As will be appreciated by the skilled artisan, the following description as applied to antibody constructs and conjugates comprising antibody constructs is applicable to recombinant bispecific antibodies and conjugates comprising recombinant bispecific antibodies

In certain embodiments, the therapeutic agent comprises a recombinant protein or monoclonal antibody. In certain embodiments, the recombinant protein or monoclonal antibody comprises Etaracizumab (Abegrin), Tacatuzumab tetraxetan, Bevacizumab (Avastin), Labetuzumab, Cetuximab (Erbitux), Obinutuzumab (Gazyva), Trastuzumab (Herceptin), Clivatuzumab, Trastuzumab emtansine (Kadcyla), Rituximab (MabThera, Rituxan), Gemtuzumab ozogamicin (Mylotarg), Girentuximab (Rencarex), or Nimotuzumab (Theracim, Theraloc). In certain embodiments, the additional therapeutic agent is a chemotherapeutic agent. In certain embodiments, the chemotherapeutic agent is an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), an inhibitor of topoisomerase (e.g., irinotecan, topotecan, amsacrine, etoposide, or teniposide), a kinase inhibitor (e.g., bortezomib, erlotinib, gefitinib, imatinib, vemurafenib, or vismodegib), a nucleoside analog or precursor analog (e.g., azacitidine, azathioprine, capecitabine, cytarabine, fluorouracil, gemcitabine, hydroxyurea, mercaptopurine, methotrexate, or thioguanine), a peptide antibiotic (e.g., actinomycin or bleomycin), a platinum-based agent (e.g., cisplatin, oxaloplatin, or carboplatin), or a plant alkaloid (e.g., vincristine, vinblastine, vinorelbine, vindesine, podophyllotoxin, paclitaxel, or docetaxel). In some embodiments, the chemotherapeutic agent is a nucleoside analog. In some embodiments, the chemotherapeutic agent is gemcitabine. In certain embodiments, the additional therapeutic agent is radiation therapy.

In some embodiments, the recombinant bispecific antibody further comprises a chemotherapeutic compound and a linker, wherein the linker links the chemotherapeutic compound to the Fc comprising domain.

In some embodiments, the chemotherapeutic compound comprises an alkylating agent, an anthracycline, a cytoskeletal disruptor, a histone deacetylase inhibitor, an inhibitor of, a kinase inhibitor, a nucleoside analog or precursor analog, a peptide antibiotic, a platinum-based compound, or a plant alkaloid.

In some aspects, a method of treatment for a subject in need thereof comprises administering a therapeutic dose of the antibody construct or conjugate as described herein or the pharmaceutical composition as described herein. In some embodiments, the subject has cancer.

In some embodiments, the antibody construct or conjugate is administered intravenously, cutaneously, subcutaneously, or injected at a site of affliction. In some embodiments, after administration of antibody construct or conjugate to the subject, immune cell activation is increased in the subject as measured by a secretion of one or more cytokines as measured by a cytokine release assay, a secretion of one or more chemokines as measured by an ELISA immunoassay, an expression level of one or more cell surface proteins associated with immune stimulation as measured by an ELISA immunoassay, an activity of one or more immune cell functions, or combination thereof, as compared to before administration of the antibody construct or conjugate to the subject.

In some embodiments, the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay.

In some embodiments, after administration of the antibody construct or conjugate to the subject, tumor cell intracellular signaling is altered in the subject as compared to tumor cell intracellular signaling before administration of the antibody construct or conjugate as measured by an intracellular signaling assay.

In some embodiments, the altered tumor cell intracellular signaling increases tumor immunogenicity as measured by an immunogencity assay.

In some aspects, a kit comprising a pharmaceutically acceptable dosage unit of a pharmaceutically effective amount of the conjugate or antibody construct as described herein or the pharmaceutical composition as described herein.

### Diseases, Conditions and the Like

The conjugates, antibody constructs, recombinant bispecific antibodies, pharmaceutical compositions thereof, and methods provided herein can be useful for the treatment of a plurality of diseases, conditions, preventing a disease or a condition in a subject or other therapeutic applications for subjects in need thereof. As will be appreciated by the skilled artisan, the following description as applied to antibody constructs and conjugates comprising antibody constructs is applicable to recombinant bispecific antibodies and conjugates comprising recombinant bispecific antibodies.

Often the conjugates, antibody constructs, pharmaceutical compositions, and methods provided herein can be useful for treatment of hyperplastic conditions, including but not limited to, neoplasms, cancers, tumors and the like. A condition, such as a cancer, can be associated with expression of a molecule on the cancer cells. Often, the molecule expressed by the cancer cells can comprise an extracellular portion capable of recognition by the antibody portion of the antibody construct. A molecule expressed by the cancer cells can be a tumor antigen. An antibody portion of the conjugate or pharmaceutical composition can recognize a tumor antigen. A tumor antigen can include CD5, CD19, CD20, CD25, CD37, CD30, CD33, CD45, CAMPATH-1, BCMA, CS-1, PD-L1, B7-H3, B7-DC, HLA-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, EGFRVIII (de2-7 EGFR), fibroblast activation protein, tenascin, a metalloproteinase, endosialin, vascular endothelial growth factor, αvβ3, WT1, LMP2, HPV E6 E7, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, EGFR, CMET, HER3, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), VTCN1 (B7-H4), STRA6, TMPRSS3, TMRRSS4, TMEM238, C1orf186, LIV1, ROR1, Fos-related antigen 1, VEGFR1, endoglin, VISTA, or a fragment thereof.

As described herein, an antigen binding domain portion of the conjugate, can be configured to recognize a molecule expressed by a cancer cell, such as for example, a disease antigen, tumor antigen or a cancer antigen. Often such antigens are known to those of ordinary skill in the art, or newly found to be associated with such a condition, to be commonly associated with, and/or, specific to, such conditions. For example, a disease antigen, tumor antigen or a cancer antigen is, but is not limited to, CD5, CD19, CD20, CD25, CD37, CD30, CD33, CD45, CAMPATH-1, BCMA, CS-1, PD-L1, B7-H3, B7-DC, HLA-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, αvβ3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, EGFR, CMET, HER3, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), VTCN1 (B7-H4), STRA6, TMPRSS3, TMRRSS4, TMEM238, C1orf186, LIV1, ROR1, Fos-related antigen 1, VEGFR1, endoglin, VISTA, or a fragment thereof. Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrim macroglobulinemia; Wilms tumor and any combination thereof.

In certain embodiments, the recombinant bispecific antibodies and conjugates comprising recombinant bispecific antibodies are useful for the treatment of a cancer or tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular and liver tumors. In certain embodiments, tumors which can be treated with the recombinant bispecific antibodies (including conjugates thereof) comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, multiple myeloma, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, vipoma, and Wilm's tumor. In certain embodiments, the tumor/cancer to be treated with one or more recombinant bispecific antibodies (including conjugates thereof) comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the recombinant bispecific antibodies (including conjugates thereof) comprises glioblastoma. In certain embodiments, the cancer treated with one or more recombinant bispecific antibodies (including conjugates thereof) comprises pancreatic cancer. In certain embodiments, the cancer treated with one or more recombinant bispecific antibodies (including conjugates thereof) comprises ovarian cancer. In certain embodiments, the cancer treated with one or more recombinant bispecific antibodies (including conjugates thereof) comprises or lung cancer.

In certain embodiments, the recombinant bispecific antibodies (including conjugates thereof) can be administered by any route suitable for the administration of antibody-containing pharmaceutical compositions, such as, for example, cutaneous, subcutaneous, intraperitoneal, intravenous, intramuscular, intratumoral, intracerebral, or at a tumor afflicted site, etc. In certain embodiments, the recombinant bispecific antibodies (including conjugates thereof) are administered intravenously. In certain embodiments, the recombinant bispecific antibodies (including conjugates thereof) are administered on a suitable dosage schedule, for example, weekly, twice weekly, three times weekly, once every two weeks, once every three weeks, monthly, twice monthly, three times monthly, etc. The recombinant bispecific antibodies (including conjugates thereof) can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. In certain embodiments, the therapeutically acceptable amount is between about 1 mg/kg and about 10 mg/kg.

### EXAMPLES

The following examples are included to further describe some embodiments of the present disclosure, and should not be used to limit the scope of the disclosure

### Example 1

### Synthesis of Linkers with Immune-Stimulatory Compounds

A linker is linked with an immune-stimulatory compound. A linker linked to an immune-stimulatory compound is formed to make a linker-immune stimulatory compound construct (ATAC). Subsequently, an ATAC is conjugated to an antibody, in which the ATAC is any one of ATAC1-ATAC34 (each of which is described in the below EXAMPLES).

A linker is linked with an antibody, in which the linker is a PEGylated linker, a linker containing a valine-alanine dipeptide, a linker containing a valine-citrulline dipeptide, or an N-Maleimidomethylcyclohexane-1-carboxylate (MCC) linker. Subsequently, an immune-stimulatory compound is conjugated to the linker linked with the antibody or antibody construct, in which the immune-stimulatory compound is a TLR agonist, a Nod-like receptor ligand, a RIG-Like receptor agonist, a CLR ligand, a CDS ligand, or an inflammasome inducer.

A linker is linked with an antibody or antibody construct, in which the linker is a PEGylated linker, a linker containing a valine-alanine dipeptide, a linker containing a valine-citrulline dipeptide, or an N-Maleimidomethylcyclohexane-1-carboxylate (MCC) linker. Subsequently, an immune-stimulatory compound is conjugated to the linker linked with the antibody or antibody construct, in which the immune-stimulatory compound is gardiquimod or an analog of a cyclic dinucleotide.

### Example 2

### Synthesis of ATAC1 and ATAC2

This example shows the synthesis of pentafluorophenyl 25-(2-amino-3-pentylquinolin-5-yl)-19-oxo-4,7,10,13,16-pentaoxa-20-azapentacosanoate (ATAC1) and Perfluorophenyl 3-((4-amino-1-(2-hydroxy-2-methylpropyl)-1H-imidazo[4,5-c]quinolin-2-yl)methyl)-4-oxo-7,10,13,16,19-pentaoxa-3-azadocosan-22-oate (ATAC2).

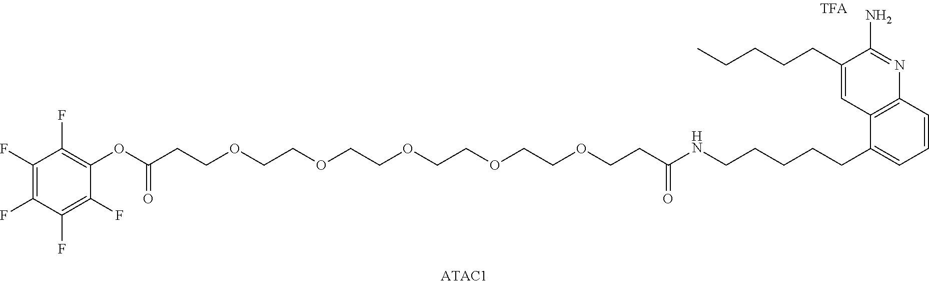 [SMILES: CCCCCc1cc2c(CCCCCNC(=O)CCOCCOCCOCCOCCOCCC(=O)Oc3c(F)c(F)c(F)c(F)c3F)cccc2nc1N]

### Step A: Preparation of Int ATAC1-1

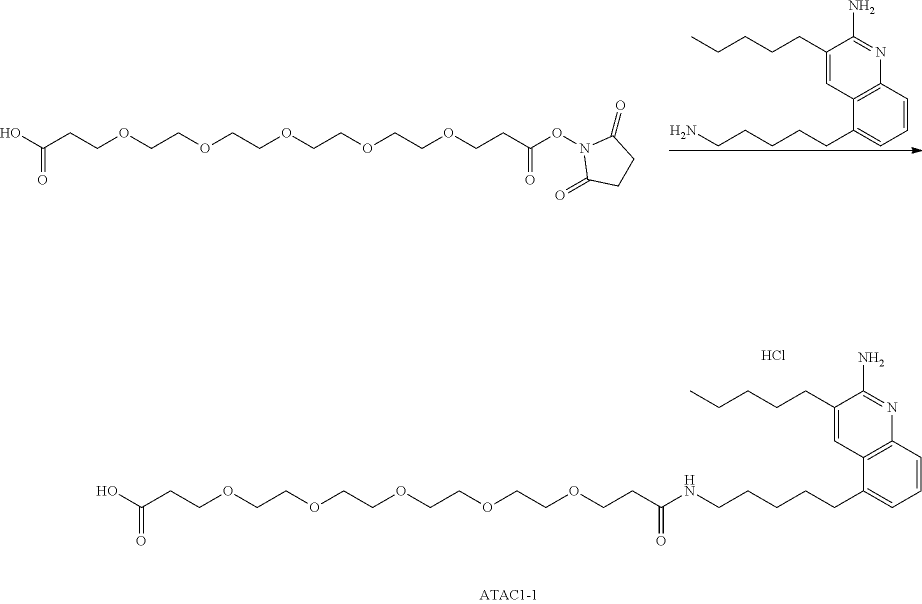 [SMILES: O=C(O)CCOCCOCCOCCOCCOCCC(=O)ON1C(=O)CCC1=O ; CCCCCc1cc2c(CCCCCN)cccc2nc1N ; Cl ; CCCCCc1cc2c(CCCCCNC(=O)CCOCCOCCOCCOCCOCCC(=O)O)cccc2nc1N]

To a 0° C. solution containing 271 mg (0.90 mmol) of 5-(5-aminopentyl)-3-pentylquinolin-2-amine in 4 mL of DCM was added 435 mg (1.00 mmol) of the NHS ester in 1 mL of DCM dropwise over 15 minutes. The reaction mixture was allowed to warm to ambient temperature over 19h before it was concentrated and purified by reverse phase chromatography. Pure fractions were lyophilized and dissolved in 3 mL of methanol then treated with 1 mL of 4N HCl in dioxane. The solution was stirred for 1h then concentrated to afford the desired compound as an HCl salt which was used directly in the next step.

### Step B: Preparation of ATAC1

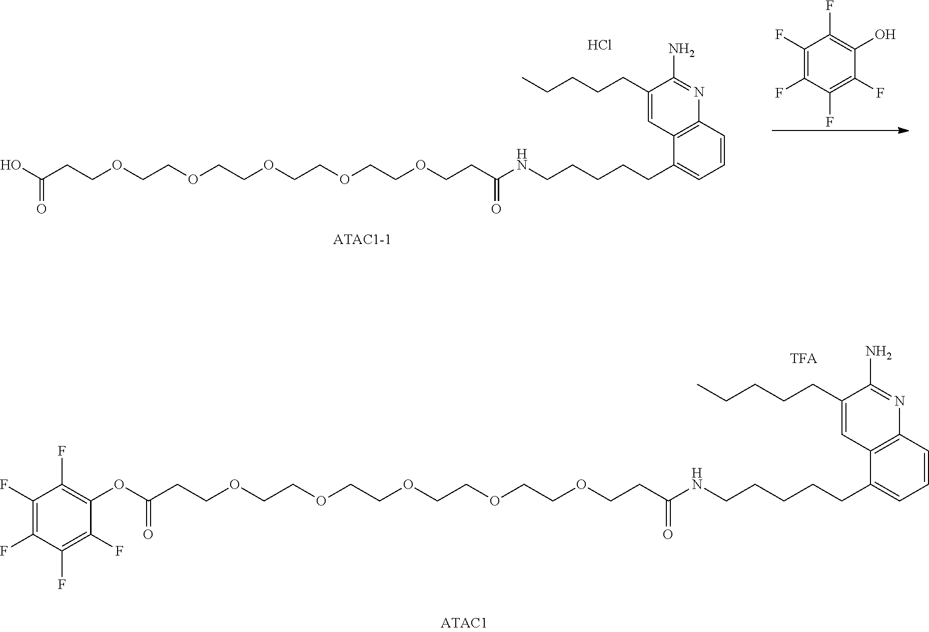 [SMILES: CCCCCc1cc2c(CCCCCNC(=O)CCOCCOCCOCCOCCOCCC(=O)O)cccc2nc1N ; Cl ; Oc1c(F)c(F)c(F)c(F)c1F ; CCCCCc1cc2c(CCCCCNC(=O)CCOCCOCCOCCOCCOCCC(=O)Oc3c(F)c(F)c(F)c(F)c3F)cccc2nc1N ; "TFA"]

To a stirred solution of 25-(2-amino-3-pentylquinolin-5-yl)-19-oxo-4,7,10,13,16-pentaoxa-20 azapentacosanoic acid hydrochloride (130 mg, 0.198 mmol) and pentafluorophenol (146 mg, 0.792 mmol) in DMF (2.5 ml) at room temperature was added N,N′-Diisopropylcarbodiimide (0.186 ml, 1.189 mmol) dropwise. The reaction was stirred at room temperature for 18h and then concentrated. The crude product was added to a 100 g C18 gold reverse phase column and was eluted with water/acetonitrile (0.1% TFA) 10-100%. The fractions were combined and concentrated then freeze dried to give perfluorophenyl 25-(2-amino-3-pentylquinolin-5-yl)-19-oxo-4,7,10,13,16-pentaoxa-20-azapentacosanoate-2,2,2-trifluoroacetate (110 mg, 61.7% yield) as a clear gum. ¹H NMR (DMSO-d⁶) δ 13.7 (s, 1H), 8.37-8.35 (m, 3H), 7.78 (t, J=5.5 Hz, 1H), 7.63 (t, J=7.5 Hz, 1H), 7.53 (d, J=8.5 Hz, 1H), 7.31 (d, J=7.0 Hz, 1H), 3.58 (t, J=6.0 Hz, 2H), 3.63-3.43 (m, 20H), 3.04-2.96 (m, 6H), 2.73 (t, J=7.5 Hz, 2H), 2.27 (t, J=7.5 Hz, 2H), 1.60-1.55 (m, 4H), 1.44-1.33 (m, 9H), 0.88 (t, J=7.5 Hz, 3H). LCMS [M+H]=786.3.

The following compound in TABLE 21 can be prepared using a method similar to that described above for ATAC1.

TABLE 21

| Com- | | | |
|---|---|---|---|
| pound | Structure | IUPAC | M + 1 |
| ATAC2 | 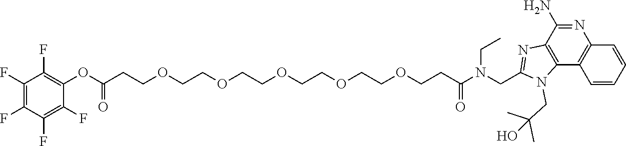 [SMILES: CCN(Cc1nc2c(N)nc3ccccc3c2n1CC(C)(C)O)C(=O)CCOCCOCCOCCOCCOCCC(=O)Oc1c(F)c(F)c(F)c(F)c1F] | Perfluorophenyl 3- ((4-amino- 1-(2-hydroxy-2- methylpropyl)-1H- imidazo[4,5-c]quinolin-2- yl)methyl)-4-oxo- 7,10,13,16,19-pentaoxa-3- azadocosan-22-oate | 800 |

### Example 3

### Synthesis of ATAC3 and ATAC4

This example shows the synthesis of pentafluorophenyl 25-(2-amino-3-pentylquinolin-5-yl)-19-oxo-4,7,10,13,16-pentaoxa-20-azapentacosanoate (ATAC3) and 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4).

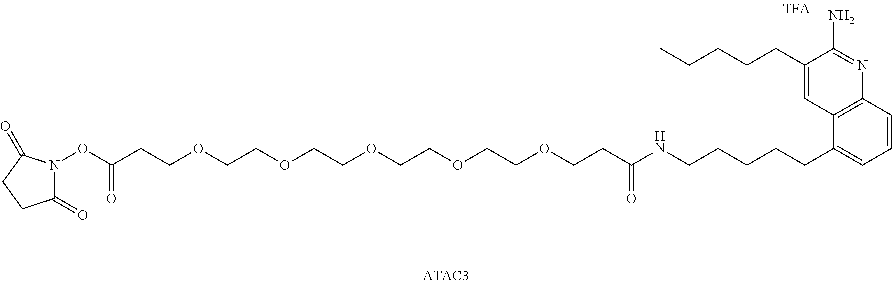 [SMILES: CCCCCc1cc2c(CCCCCNC(=O)CCOCCOCCOCCOCCOCCC(=O)ON3C(=O)CCC3=O)cccc2nc1N]

### Step A: Preparation of ATAC3

To a stirred solution of Int ATAC1-1 (185 mg, 0.282 mmol) and N-hydroxysuccinimide (130 mg, 1.128 mmol) in DMF (3 ml) was added N,N′-diisopropylcarbodiimide (0.221 ml, 1.411 mmol) dropwise and the reaction mixture was stirred at room temperature for 18 hours. The reaction mixture was filtered and washed with acetonitrile and the filtrate was evaporated. The resulting residue was purified by silica gel Silica gel column chromatography (DCM/MeOH/HOAc) to give 65 mg of the desired product as the acetic acid salt which was subsequently dissolved in 2 mL of DCM and treated with 2M HCl in diethyl ether. The solution was stirred for 1h then concentrated and lyophilized to afford the desired compound as the HCl salt. ¹H NMR (CDCl₃) δ 15.2 (s, 1H), 8.15 (d, J=7.8 Hz, 1H), 7.68 (d, J=7.9 Hz, 1H), 7.55 (t, J=8.1 Hz, 1H), 6.55 (bs, 1H), 3.98 (t, J=6.0 Hz, 2H), 3.83-3.55 (m, 18H), 3.33-3.22 (m, 2H), 2.95-2.56 (m, 11H), 2.27 (t, J=7.5 Hz, 2H), 1.60-1.55 (m, 4H), 1.44-1.33 (m, 9H), 0.88 (t, J=7.5 Hz, 3H). LCMS [M+H]=717.3.

The following 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4) compound can be prepared using a method similar to that described above for ATAC3.

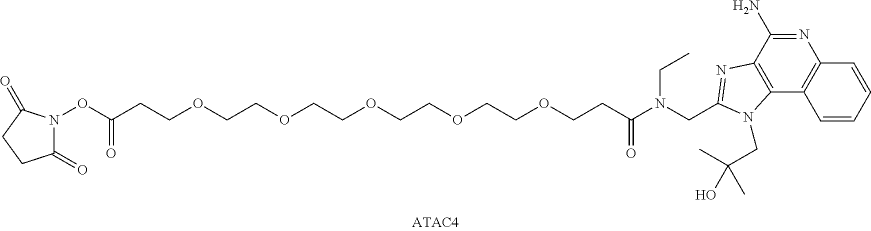 [SMILES: CCN(Cc1nc2c(N)nc3ccccc3c2n1CC(C)(C)O)C(=O)CCOCCOCCOCCOCCOCCC(=O)ON1C(=O)CCC1=O]

¹H NMR (CDCl₃) δ 14.9 (s, 1H), 8.88 (bs, 1H), 8.15 (d, 1H), 7.85 (d, 1H), 7.61 (t, 1H), 7.45 (t, 1H), 4.72 (s, 2H), 3.83 (m, 4H), 3.65-3.45 (m, 18H), 2.90-2.71 (m, 9H), 1.43 (t, J=7.0 Hz, 3H), 1.33 (s, 6H). LCMS [M+H]=731.

### Example 4

### Synthesis of ATAC5, ATAC6 and ATAC7

This example shows the synthesis of 2,5-dioxopyrrolidin-1-yl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC5), 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC6), and 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC7).

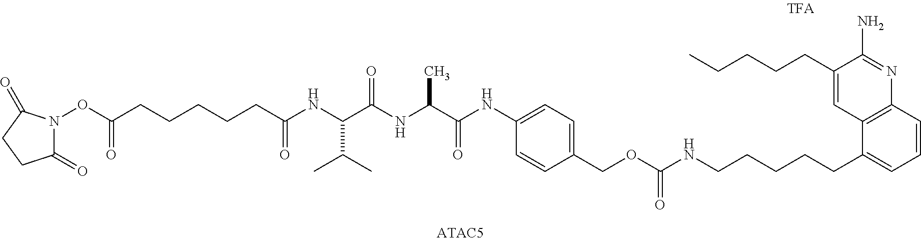 [SMILES: CCCCCc1cc2c(CCCCCNC(=O)OCc3ccc(NC(=O)[C@H](C)NC(=O)[C@@H](NC(=O)CCCCCC(=O)ON4C(=O)CCC4=O)C(C)C)cc3)cccc2nc1N]

### Step A: Preparation of Int ATAC5-1

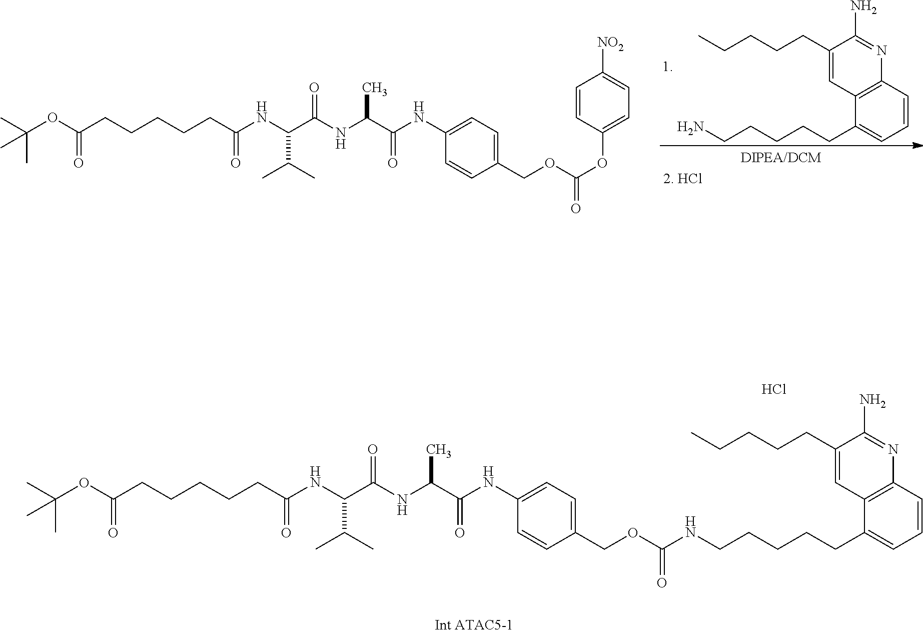 [SMILES: CC(C)[C@H](NC(=O)CCCCCC(=O)OC(C)(C)C)C(=O)N[C@@H](C)C(=O)Nc1ccc(COC(=O)Oc2ccc([N+](=O)[O-])cc2)cc1 ; CCCCCc1cc2c(CCCCCN)cccc2nc1N ; Cl ; CCCCCc1cc2c(CCCCCNC(=O)OCc3ccc(NC(=O)[C@H](C)NC(=O)[C@@H](NC(=O)CCCCCC(=O)OC(C)(C)C)C(C)C)cc3)cccc2nc1N]

A solution of 5-(5-aminopentyl)-3-pentylquinolin-2-amine (300 mg, 1.00 mmol) in 5 mL DCM was stirred at room temperature under nitrogen for 10 min before tert-butyladipate-valine-alanine-para-aminobenzyl-4-nitrophenylcarbonate (tBuAdip-va-PAB-OPNP, 656 mg, 1.00 mmol) and DIPEA (0.26 ml, 1.5 mmol) in 3 mL of DCM were added and the mixture was stirred at room temperature overnight. The mixture was concentrated and purified by column chromatography. Clean fractions were combined and evaporated and the residue was dissolved in 2 mL of DCM and treated with 2M HCl in diethyl ether. The solution was stirred for 1h then concentrated and lyophilized to afford the desired compound Int ATAC5-1 as the HCl salt. MS m/z 761 (M)⁺.

### Step B: Preparation of ATAC5

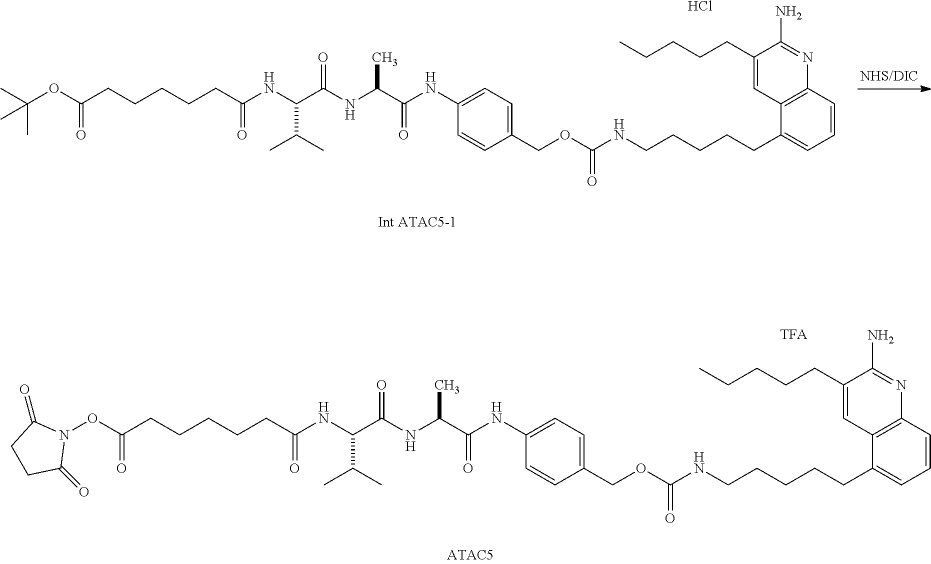

To a stirred solution of 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoic acid hydrochloride (221 mg, 0.282 mmol) and N-hydroxysuccinimide (130 mg, 1.12 mmol) in DMF (3 ml) was added N,N′-diisopropylcarbodiimide (0.221 ml, 1.41 mmol) dropwise and the reaction mixture was stirred at room temperature for 5h. HPLC indicated some starting material remained so the reaction was stirred at ambient temperature overnight. The reaction mixture was filtered and washed with acetonitrile. The filtrate was evaporated and the residue was dissolved in DMSO and purified by reverse phase chromatography [water/acetonitrile (0.1% TFA)] from 10% followed by a gradient from 20 to 80%. Pure fractions were combined to give 2,5-dioxopyrrolidin-1-yl 6-(((S)-1-(((S)-1-((4-((((5-(2-amino-3-pentylquinolin-5-yl)pentyl)-carbamoyl)oxy)methyl)phenyl)amino)-1-oxopropan-2-yl)amino)-3-methyl-1-oxobutan-2-yl)amino)-6-oxohexanoate 2,2,2-trifluoroacetate (109 mg, 40% yield) as a white solid. ¹H NMR (DMSO-d⁶) δ 13.6 (s, 1H), 9.92 (s, 1H), 8.32 (d, J=7.5 Hz, 1H), 7.84 (d, J=8.5 Hz, 1H), 7.61-7.55 (m, 4H), 7.30-7.17 (m, 4H), 4.92 (s, 2H), 4.37 (t, J=7.0 Hz, 1H), 4.18 (t, J=7.0 Hz, 1H), 2.96 (m, 4H), 2.81-2.62 (m, 8H), 2.33-2.11 (m, 2H), 1.95 (q, J=7.0 Hz, 1H), 1.63-1.55 (m, 8H), 1.50-1.40 (m, 2H), 1.38-1.33 (m, 4H), 1.29 (d, J=7.0 Hz, 3H), 0.83 (d, J=7.0 Hz, 6H). LCMS [M+H]=844.3.

The following ATAC6 compound and ATAC7 compound in TABLE 22 can be prepared using a method similar to that described above for ATAC5.

TABLE 22

| Com- | | | |
|---|---|---|---|
| pound | Structure | Name | M + 1 |
| ATAC6 | 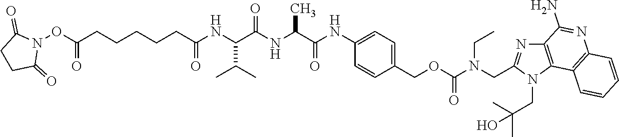 [SMILES: CCN(Cc1nc2c(N)nc3ccccc3c2n1CC(C)(C)O)C(=O)OCc1ccc(NC(=O)[C@H](C)NC(=O)[C@@H](NC(=O)CCCCCC(=O)ON2C(=O)CCC2=O)C(C)C)cc1] | 2,5-dioxopyrrolidin- 1-yl 7-(((S)-1-(((S)-1- ((4-(((((4- amino-1-(2-hydroxy- 2-methylpropyl)-1H- imidazo[4,5-c] quinolin-2- yl)methyl)(ethyl) carbamoyl)oxy)methyl) | 872 |
| | | phenyl)amino)-1- | |
| | | oxopropan-2-yl) | |
| | | amino)-3- | |
| | | methyl-1-oxobutan- | |
| | | 2-yl)amino)-7- | |
| | | oxoheptanoate | |
| ATAC7 | 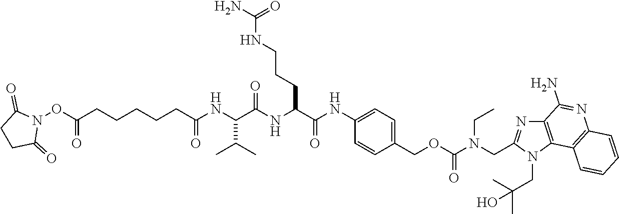 [SMILES: CCN(Cc1nc2c(N)nc3ccccc3c2n1CC(C)(C)O)C(=O)OCc1ccc(NC(=O)[C@H](CCCNC(N)=O)NC(=O)[C@@H](NC(=O)CCCCCC(=O)ON2C(=O)CCC2=O)C(C)C)cc1] | 2,5-dioxopyrrolidin- 1-yl 7-(((S)-1-(((S)-1- ((4-(((((4-amino-1- (2-hydroxy-2- methylpropyl)-1H- imidazo[4,5-c] quinolin-2- yl)methyl)(ethyl) carbamoyl)oxy)methyl) phenyl)amino)-1- oxo-5-ureidopentan- 2-yl)amino)-3- methyl-1- | 958 |
| | | oxobutan-2-yl) | |
| | | amino)-7- | |
| | | oxoheptanoate | |

### Example 5

### Synthesis of ATAC8, ATAC9, and ATAC10

This example shows synthesis of Perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC8), perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC9), and perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC10).

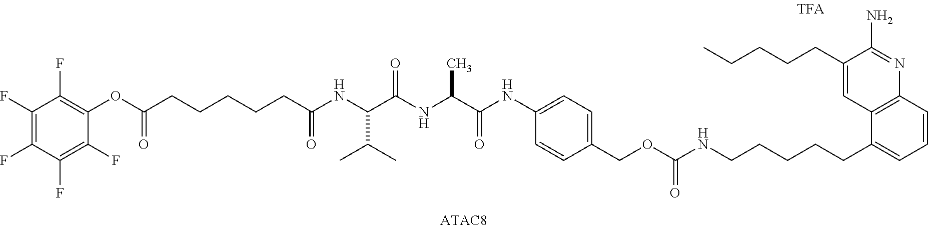 [SMILES: CCCCCc1cc2c(CCCCCNC(=O)OCc3ccc(NC(=O)[C@H](C)NC(=O)[C@@H](NC(=O)CCCCCC(=O)Oc4c(F)c(F)c(F)c(F)c4F)C(C)C)cc3)cccc2nc1N]

### Step A: Preparation of ATAC8

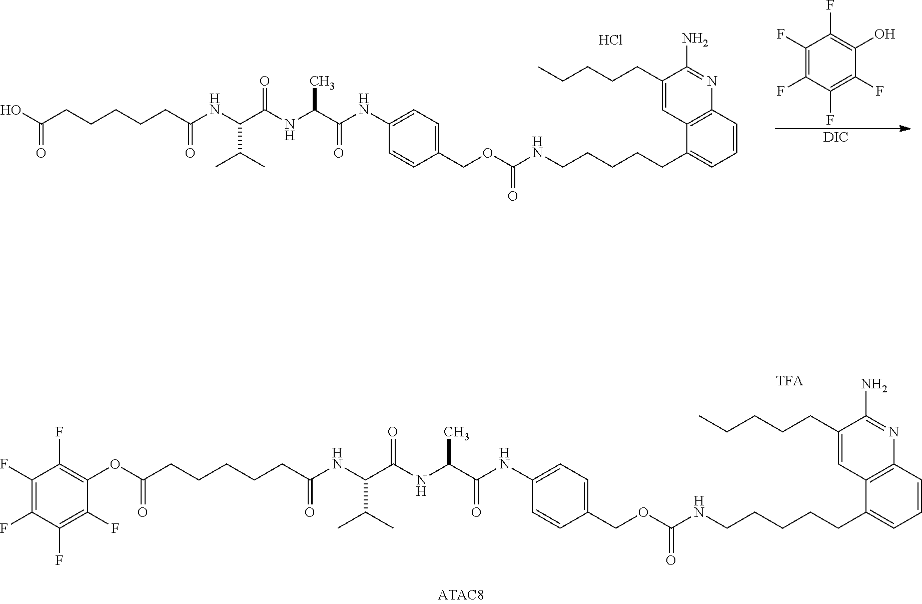

To a stirred solution of 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoic acid hydrochloride (168 mg, 0.215 mmol) and pentafluorophenol (158 mg, 0.86 mmol) in DMF (3 ml) was added N,N′-diisopropylcarbodiimide (0.166 ml, 1.07 mmol) dropwise and the reaction mixture was stirred at room temperature for 6h. The reaction mixture was concentrated and the residue was dissolved in DMSO and purified by reverse phase chromatography [water/acetonitrile (0.1% TFA)] from 10% followed by a gradient from 20 to 80%. Pure fractions were combined to give perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate 2,2,2-trifluoroacetate (122 mg) as a white solid. ¹H NMR (DMSO-d⁶) δ 13.5 (s, 1H), 9.92 (s, 1H), 8.35 (bs, 3H), 8.17 (d, J=7.0 Hz, 1H), 7.87 (d, J=7.0 Hz, 1H), 7.64-7.52 (m, 4H), 7.32-7.18 (m, 4H), 4.91 (s, 2H), 4.37 (t, J=7.0 Hz, 1H), 4.19 (t, J=7.0 Hz, 1H), 3.60-3.50 (m, 4H), 2.97 (m, 4H), 2.79 (t, J=7.0 Hz, 2H), 2.74 (t, J=7.0 Hz, 2H), 2.31-2.22 (m, 2H), 1.96 (q, J=7.0 Hz, 1H), 1.71-1.51 (m, 8H), 1.45-1.38 (m, 2H), 1.40-1.27 (m, 9H), 0.90-0.80 (m, 9H). LCMS [M+H]=913.4.

The following compounds in TABLE 23 can be prepared using a method similar to that described in above for ATAC8.

TABLE 23

| Compound | Structure | Name | M + 1 |
|---|---|---|---|
| ATAC9 | 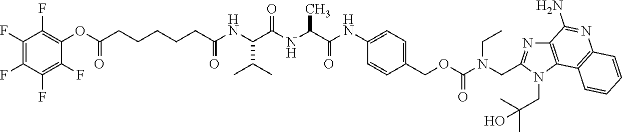 [SMILES: CCN(Cc1nc2c(N)nc3ccccc3c2n1CC(C)(C)O)C(=O)OCc1ccc(NC(=O)[C@H](C)NC(=O)[C@@H](NC(=O)CCCCCC(=O)Oc2c(F)c(F)c(F)c(F)c2F)C(C)C)cc1] | perfluorophenyl 7- (((S)-1-(((S)- 1-((4-(((((4- amino-1-(2- hydroxy-2- methylpropyl)-1H- imidazo[4,5-c] quinolin-2- yl)methyl)(ethyl) | 941 |
| | | carbamoyl) | |
| | | oxy)methyl) | |
| | | phenyl)amino)-1- | |
| | | oxopropan-2- | |
| | | yl)amino)-3- | |
| | | methyl-1- | |
| | | oxobutan-2-yl)amino)- | |
| | | 7-oxoheptanoate | |
| ATAC10 | 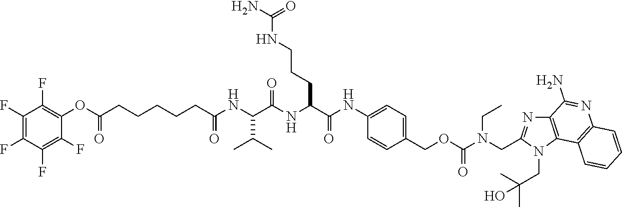 [SMILES: CCN(Cc1nc2c(N)nc3ccccc3c2n1CC(C)(C)O)C(=O)OCc1ccc(NC(=O)[C@H](CCCNC(N)=O)NC(=O)[C@@H](NC(=O)CCCCCC(=O)Oc2c(F)c(F)c(F)c(F)c2F)C(C)C)cc1] | perfluorophenyl 7-(((S)-1-(((S)- 1-((4-(((((4- amino-1-(2- hydroxy-2- methylpropyl)- 1H-imidazo[4,5-c] quinolin-2- yl)methyl)(ethyl) carbamoyl) oxy)methyl)phenyl) amino)-1-oxo-5- ureidopentan-2-yl) | 1027 |
| | | amino)-3- | |
| | | methyl-1-oxobutan- | |
| | | 2-yl)amino)- | |
| | | 7-oxoheptanoate | |

### Example 6

### Synthesis of ATAC11

This example shows the synthesis of N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (ATAC11).

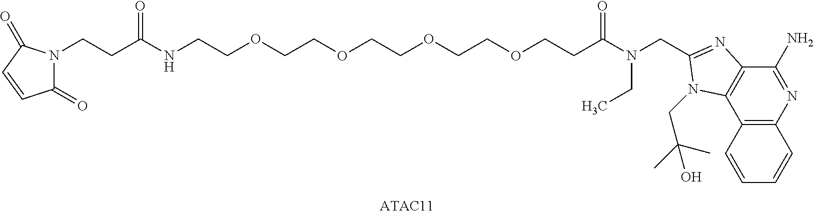 [SMILES: CCN(Cc1nc2c(N)nc3ccccc3c2n1CC(C)(C)O)C(=O)CCOCCOCCOCCOCCNC(=O)CCN1C(=O)C=CC1=O]

### Step A: Preparation of ATAC11

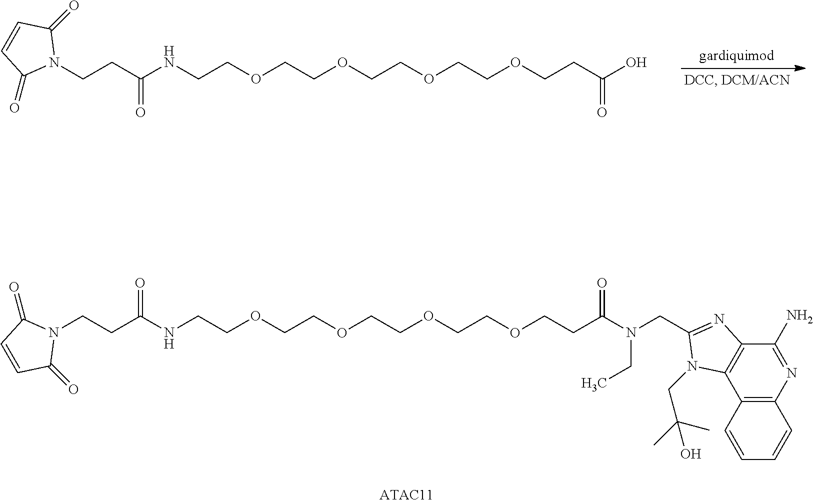 [SMILES: O=C(O)CCOCCOCCOCCOCCNC(=O)CCN1C(=O)C=CC1=O ; CCN(Cc1nc2c(N)nc3ccccc3c2n1CC(C)(C)O)C(=O)CCOCCOCCOCCOCCNC(=O)CCN1C(=O)C=CC1=O]

A solution of MAL-PEG4-acid (265.7 mg, 0.638 mmol) and N,N′-dicyclohexylcarbodiimide (DCC, 144.8 mg, 0.702 mmol) in dry dichloromethane/acetonitrile (1:1, 5 mL) was stirred at room temperature for 1h, followed by addition of compound 1 (100 mg, 0.319 mmol) in one portion. After 72h of stirring, volatile organics were removed under vacuum. The residue obtained was purified by flash column chromatography on silica gel, eluting with step gradients of methanol in dichloromethane at a ratio of v/v 1:20, 1:15, and 1:9, to afford the target product N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (80 mg, 35% yield) as white colored foamy solid oil. ¹H NMR (300 MHz, CDCl₃) δ 8.40-7.82 (br m, 1H), 7.74 (d, J=8.1 Hz, 1H), 7.44 (t, J=7.5 Hz, 1H), 7.30 (t, J=7.4 Hz, 1H), 6.76-6.28 (br m, 2H), 4.82-4.32 (br m, 2H), 4.08-3.64 (br m, 6H), 3.54 (br s, 14H), 3.31 (br s, 3H), 2.63 (br s, 2H), 2.38 (t, J=6.9 Hz, 2H), 1.27 (br s, 4H), 1.20-0.68 (br m, 5H). MS (ESI+) m/z 712 (M+1), 734 (M+Na).

### Example 7

### Synthesis of ATAC12, ATAC13, ATAC14, ATAC15, ATAC16, ATAC17, ATAC18, ATAC19, ATAC20, and ATAC21

This example shows the synthesis of N-(5-(2-amino-3-pentylquinolin-5-yl)pentyl)-1-(3-(2,5-dioxo-2,5-dihydro-1H-pyrrol-1-yl)propanamido)-3,6,9,12-tetraoxapentadecan-15-amide (ATAC12), 1-(3-(2,5-dioxo-2,5-dihydro-1H-pyrrol-1-yl)propanamido)-N-(3-pentylquinolin-2-yl)-3,6,9,12-tetraoxapentadecan-15-amide (ATAC13), 1-(3-(2,5-dioxo-2,5-dihydro-1H-pyrrol-1-yl)propanamido)-N-(1-isobutyl-1H-imidazo[4,5-c]quinolin-4-yl)-3,6,9,12-tetraoxapentadecan-15-amide (ATAC14), 1-(3-(2,5-dioxo-2,5-dihydro-1H-pyrrol-1-yl)propanamido)-N-methyl-N-(2-(3-(7-methylbenzo[1,2-d:3,4-d′]bis(thiazole)-2-yl)ureido)ethyl)-3,6,9,12-tetraoxapentadecan-15-amide (ATAC15), (S)-1-(3-(2,5-dioxo-2,5-dihydro-1H-pyrrol-1-yl)propanamido)-N-(1-((7-methylbenzo[1,2-d:3,4-d′]bis(thiazole)-2-yl)amino)-1-oxo-3-phenylpropan-2-yl)-3,6,9,12-tetraoxapentadecan-15-amide (ATAC16), N-(benzo[d]thiazol-2-yl)-1-(3-(2,5-dioxo-2,5-dihydro-1H-pyrrol-1-yl)propanamido)-N-((8-hydroxyquinolin-7-yl)(4-(trifluoromethoxy)phenyl)methyl)-3,6,9,12-tetraoxapentadecan-15-amide (ATAC17), N-((2R,3R,3aS,7aR,9R,10R,10aS,14aR)-2,9-bis(2-amino-6-oxo-1H-purin-9(6H)-yl)-5,10,12-trihydroxy-5,12-dioxidodecahydrodifuro[3,2-d:3′,2′-j][1,3,7,9,2,8]tetra-oxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC18), N-((2R,3R,3aS,7aR,9R,10R,10aS,14aR)-2,9-bis(2-amino-6-oxo-1H-purin-9(6H)-yl)-10-hydroxy-5,12-dimercapto-5,12-dioxidodecahydrodifuro[3,2-d:3′,2′-j][1,3,7,9,2,8]tetraoxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC19), N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetra-oxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC20), and N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetraoxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC21).

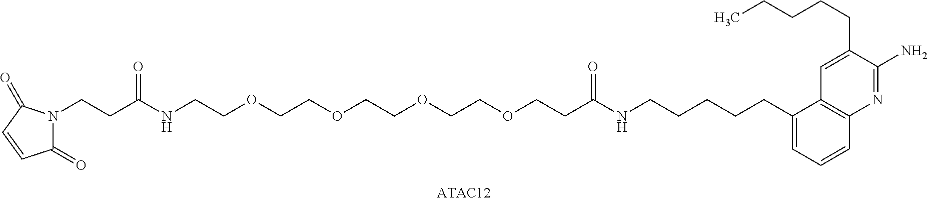 [SMILES: CCCCCc1cc2c(CCCCCNC(=O)CCOCCOCCOCCOCCNC(=O)CCN3C(=O)C=CC3=O)cccc2nc1N]

### Step A: Preparation of ATAC12

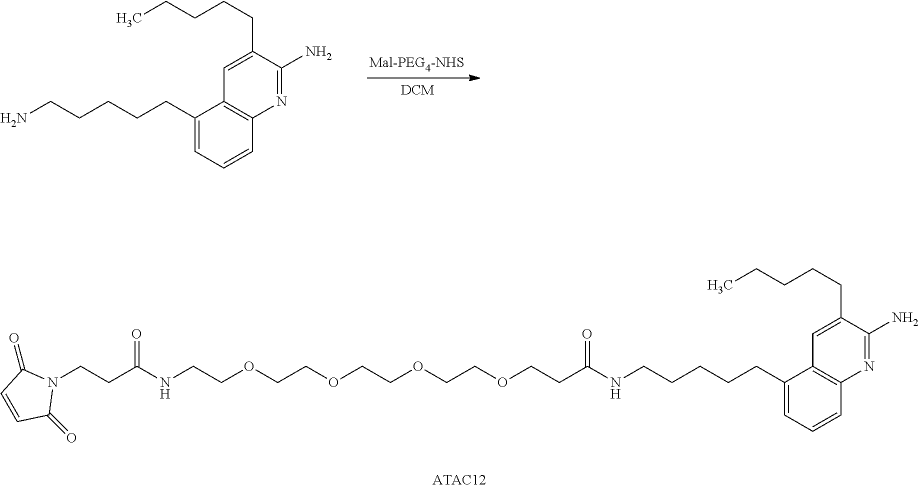 [SMILES: CCCCCc1cc2c(CCCCCN)cccc2nc1N ; CCCCCc1cc2c(CCCCCNC(=O)CCOCCOCCOCCOCCNC(=O)CCN3C(=O)C=CC3=O)cccc2nc1N]

To a stirred solution containing 100 mg (0.33 mmol) of 5-(5-aminopentyl)-3-pentylquinolin-2-amine in 13 mL of CH₂Cl₂ under N₂ was added a solution of MAL-PEG4-NHS [CAS No 756525-99-2] (171 mg, 0.33 mmol) in 3 mL of CH₂Cl₂ by syringe pump over 90 mins. The reaction mixture was stirred at room temperature for 16h then evaporated to afford a residue which was purified by silica gel chromatography (CombiFlash Gold (12g): CH₂Cl₂/CH₃OH/NH₄OH) to afford a light yellow syrup which was dissolved in 5 mL of CH₃CN and lyophilized to provide 164 mg of the desired compound. ¹H NMR (CD₃OD) δ 7.95 (s, 1H), 7.38 (s, 1H), 7.37 (s, 1H), 7.07 (t, J=8.5 Hz, 1H), 6.78 (s, 2H), 3.75 (t, J=6.0 Hz, 2H), 3.65 (t, J=6.0 Hz, 2H), 3.59-3.52 (m, 12H), 3.46 (t, J=5.5 Hz, 2H), 3.28 (t, J=7.5 Hz, 2H), 3.18 (t, J=7.5 Hz, 2H), 2.98 (t, J=8.5 Hz, 2H), 2.67 (t, J=7.5 Hz, 2H), 2.44 (t, J=7.0 Hz, 2H), 2.40 (t, J=7.0 Hz, 2H), 1.76-1.68 (m, 4H), 1.58-1.52 (m, 2H), 1.46-1.40 (m, 6H), 0.94 (t, J=7.0 Hz, 3H). (MS (ESI+) m/z 698 (M+1).

The following compounds in TABLE 24 can be prepared using a method similar to that as described above for ATAC12.

TABLE 24

| Compound | Structure | Name | M + 1 |
|---|---|---|---|
| ATAC13 | 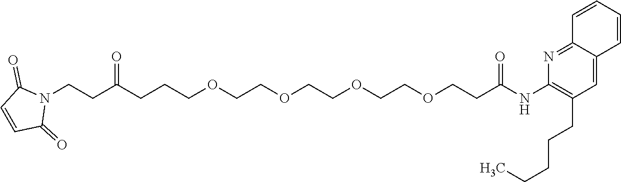 [SMILES: CCCCCc1cc2ccccc2nc1NC(=O)CCOCCOCCOCCOCCCC(=O)CCN1C(=O)C=CC1=O] | 1-(3-(2,5-dioxo- 2,5-dihydro-1H- pyrrol-1-yl) propanamido)- N-(3-pentyl- quinolin- 2-yl)-3,6,9,12- tetraoxapenta- decan-15-amide | 613 |
| ATAC14 | 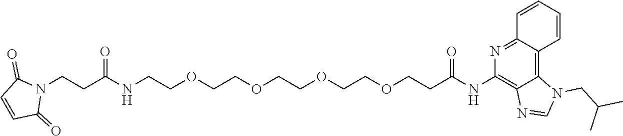 [SMILES: CC(C)Cn1cnc2c(NC(=O)CCOCCOCCOCCOCCNC(=O)CCN3C(=O)C=CC3=O)nc3ccccc3c21] | 1-(3-(2,5-dioxo- 2,5-dihydro-1H- pyrrol-1-yl) propanamido)- N-(1-isobutyl- 1H-imidazo[4,5- c]quinolin-4-yl)- 3,6,9,12- tetraoxapenta- decan-15-amide | 639 |
| ATAC15 | 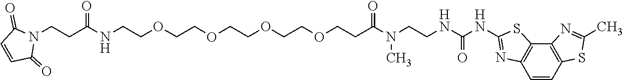 [SMILES: Cc1nc2c(ccc3nc(NC(=O)NCCN(C)C(=O)CCOCCOCCOCCOCCNC(=O)CCN4C(=O)C=CC4=O)sc32)s1] | 1-(3-(2,5-dioxo- 2,5-dihydro-1H- pyrrol-1-yl) propanamido)-N- methyl-N-(2-(3- (7-methylbenzo | 720 |
| | | [1,2-d:3,4- | |
| | | d′]bis(thiazole)- | |
| | | 2-yl)ureido)ethyl)- | |
| | | 3,6,9,12-tetra- | |
| | | oxapentadecan-15- | |
| | | amide | |
| ATAC16 | 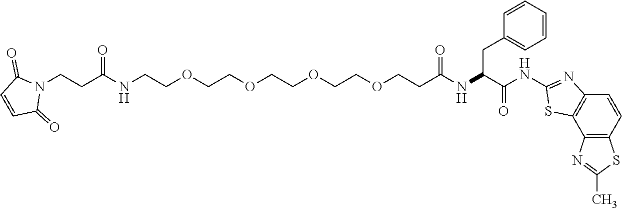 [SMILES: Cc1nc2c(ccc3nc(NC(=O)[C@H](Cc4ccccc4)NC(=O)CCOCCOCCOCCOCCNC(=O)CCN4C(=O)C=CC4=O)sc32)s1] | (S)-1-(3-(2,5- dioxo-2,5- dihydro-1H- pyrrol-1-yl) propanamido)- N-(1-((7- methylbenzo [1,2-d:3,4- d′]bis(thiazole)- 2-yl)amino)-1- oxo-3- phenylpropan- 2-yl)-3,6,9,12- | 635 |
| | | tetraoxapenta- | |
| | | decan-15-amide | |
| ATAC17 | 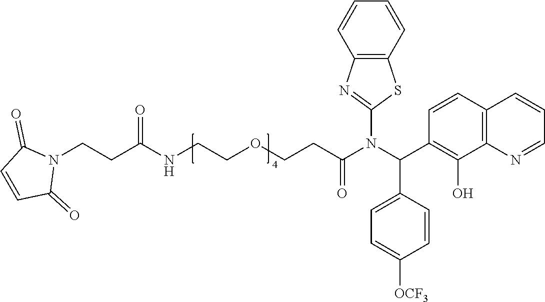 | N-(benzo[d] thiazol-2-yl)- 1-(3-(2,5-dioxo- 2,5-dihydro-1H- pyrrol-1-yl) propanamido)- N-((8- hydroxyquinolin- 7-yl)(4- (trifluoromethoxy) phenyl)methyl)- 3,6,9,12- tetraoxapenta- decan-15- amide | 734 |
| ATAC18 | 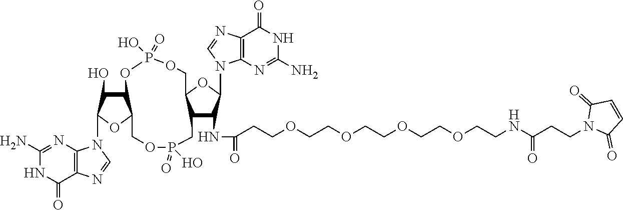 | N- ((2R,3R,3aS,7aR, 9R,10R,10aS,14aR)- 2,9-bis(2-amino- 6-oxo-1H-purin- 9(6H)-yl)-5,10,12- trihydroxy-5,12- dioxidodeca- hydrodifuro[3,2- d:3′,2′-j] [1,3,7,9,2,8]tetra- oxadiphospha- cyclododecin-3-yl)- | 1088 |
| | | 1-(3-(2,5-dioxo- | |
| | | 2,5-dihydro-1H- | |
| | | pyrrol-1-yl) | |
| | | propanamido)- | |
| | | 3,6,9,12- | |
| | | tetraoxapenta- | |
| | | decan-15-amide | |
| ATAC19 | 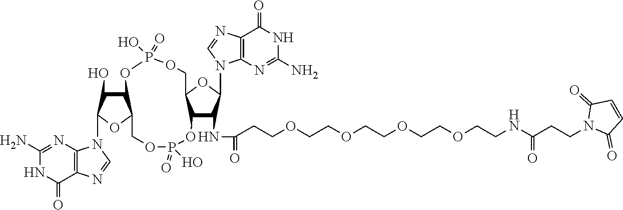 | N- ((2R,3R,3aS,7aR, 9R,10R,10aS, 14aR)-2,9-bis(2- amino-6-oxo-1H- purin-9(6H)-yl)- 10-hydroxy-5,12- dimercapto-5,12- dioxidodeca- hydrodifuro[3,2- d:3′,2′-j] [1,3,7,9,2,8] tetraoxadiphospha- | 1120 |
| | | cyclododecin-3- | |
| | | yl)-1-(3-(2,5-dioxo- | |
| | | 2,5-dihydro-1H- | |
| | | pyrrol-1-yl) | |
| | | propanamido)- | |
| | | 3,6,9,12- | |
| | | tetraoxapenta- | |
| | | decan-15-amide | |
| ATAC20 | 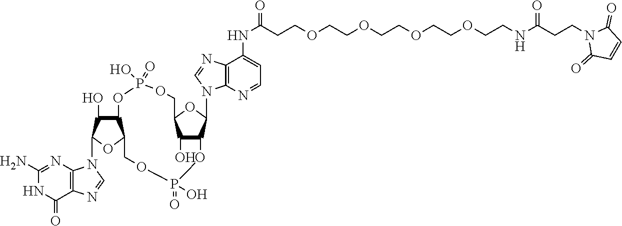 | N-(9- ((2R,3R,3aS,7aR, 9R,10R,10aS,14 aR)-9-(2-amino- 6-oxo-1H-purin- 9(6H)-yl)- 3,5,10,12- tetrahydroxy- 5,12-dioxidodeca- hydrodifuro[3,2- d:3′,2′-j] [1,3,7,9,2,8]tetra- oxadiphospha- cyclododecin-2- | 1073 |
| | | yl)-9H-purin-6- | |
| | | yl)-1-(3-(2,5- | |
| | | dioxo-2,5-dihydro- | |
| | | 1H-pyrrol-1-yl) | |
| | | propanamido)- | |
| | | 3,6,9,12- | |
| | | tetraoxapenta- | |
| | | decan-15-amide | |
| ATAC21 | 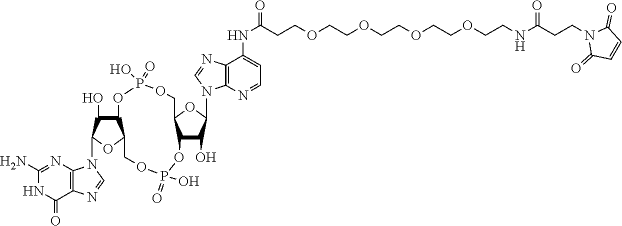 | N-(9- ((2R,3R,3aS,7aR, 9R,10R,10aS, 14aR)-9-(2-amino- 6-oxo-1H-purin- 9(6H)-yl)-3,5, 10,12-tetrahydroxy- 5,12-dioxidodeca- hydrodifuro[3,2- d:3′,2′-j] [1,3,7,9,2,8] tetraoxadiphospha- cyclododecin-2- yl)-9H-purin-6-yl)- | 1073 |
| | | 1-(3-(2,5-dioxo- | |
| | | 2,5-dihydro-1H- | |
| | | pyrrol-1-yl) | |
| | | propanamido)- | |
| | | 3,6,9,12- | |
| | | tetraoxapenta- | |
| | | decan-15-amide | |

### Example 8

### Synthesis of ATAC22, ATAC23, ATAC24, ATAC25, ATAC26, ATAC27, ATAC28, ATAC29, ATAC30, and ATAC31

This example shows the synthesis of 4-((S)-2-((S)-2-(6-(2,5-dioxo-2,5-dihydro-1H-pyrrol-1-yl)hexanamido)-3-methylbutanamido)propanamido)benzyl ((4-amino-1-(2-hydroxy-2-methyl-propyl)-1H-imidazo[4,5-c]quinolin-2-yl)methyl)(ethyl)carbamate (ATAC22), 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate (ATAC23), 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate (ATAC24), 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt (ATAC25), 2-(3-{2-[N-Methyl({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino]ethyl}ureido)-7-methyl-1,6-dithia-3,8-diaza-as-indacene (ATAC26), 2-{[(8-Hydroxy-7-quinolyl)(p-trifluoromethoxyphenyl)methyl] ({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino}-1,3-benzothiazole (ATAC27), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC28), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}propionylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC29), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-9-hydroxy-3,12-dimercapto-2.4.7.11.13.16-hexaoxa-3λ5.12λ5-diphosphatricyclo[13.3.0.06,10]octadecane-3,12-dione (ATAC30), and {p-[(S)-2-{(S)-2-[6-(2,5-Dioxo-1H-pyrrol-1-yl)hexanoylamino]-3-methylbutyrylamino}-5-ureidovalerylamino]phenyl}methyl 9-{(1S,6R,8R,9S,10S,15R,17R,18S)-8-(2-amino-6-oxo-1,9-dihydropurin-9-yl)-3,12-dihydroxy-9,18-dihydroxy-3,12-dioxo-2.4.7.11.13.16-hexaoxa-3λ5.12λ5-diphosphatricyclo[13.2.1.06,10]octadec-17-yl}-9a-adenineecarboxylate (ATAC31).

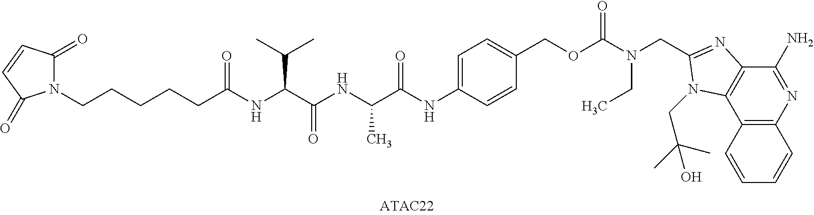 [SMILES: CCN(Cc1nc2c(N)nc3ccccc3c2n1CC(C)(C)O)C(=O)OCc1ccc(NC(=O)[C@H](C)NC(=O)[C@@H](NC(=O)CCCCCN2C(=O)C=CC2=O)C(C)C)cc1]

### Step A: Preparation of ATAC22

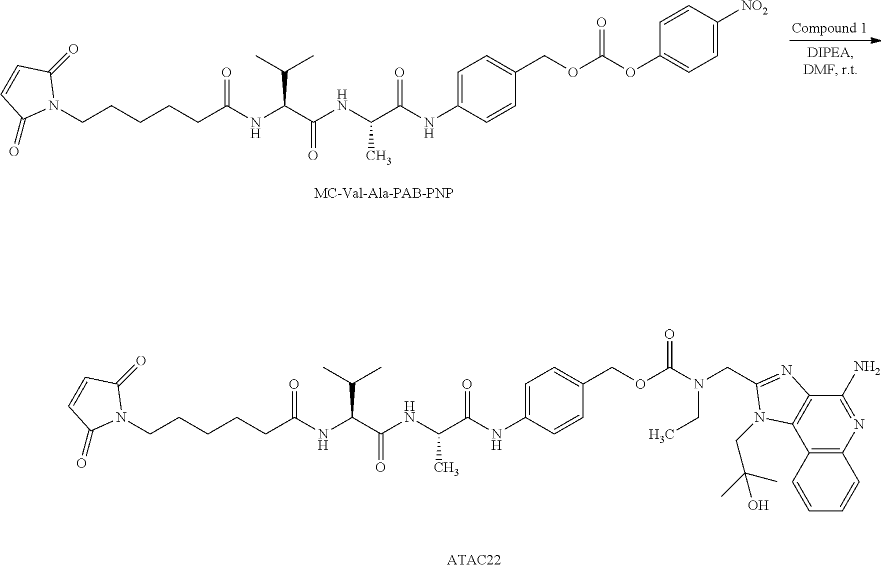 [SMILES: CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CCN(Cc1nc2c(N)nc3ccccc3c2n1CC(C)(C)O)C(=O)OCc1ccc(NC(=O)[C@H](C)NC(=O)[C@@H](NC(=O)CCCCCN2C(=O)C=CC2=O)C(C)C)cc1]

A solution of compound 1 (150 mg, 0.479 mmol) and N,N′-diisopropylethylamine (145.4 mg, 1.437 mmol) in dry DMF was stirred at room temperature for 5 min., followed by addition of maleimidocaproyl-valine-alanine-p-aminobenzyl alcohol p-nitrophenyl-carbonate (MC-Val-Ala-PAB-PNP, 343.6 mg, 0.527 mmol). After stirring for 24 h, volatile organics were removed under vacuum. The residue obtained was triturated with dry acetonitrile. The precipitated solid was collected by filtration, washed with acetonitrile and dried under vacuum to obtain unreacted MC-Val-Ala-PAB-PNP (130 mg) as beige solid. The filtrate and washings were combined and concentrated under vacuum. The residue obtained was purified by flash column chromatography on silica gel, eluting with step gradients of MeOH in dichloromethane at a ratio of v/v 1:20, 1:15, and 1:10, to afford the target product mc-Val-Ala-PAB-GDQ (70 mg, 18% yield) as beige colored foamy solid. ¹H NMR (DMSO-d⁶) δ 10.1-9.75 (br m, 1H), 8.58-8.24 (br m, 1H), 8.15 (d, J=6.6 Hz, 1H), 8.01 (br s, 1H), 7.81 (d, J=8.4 Hz, 1H), 7.71 (d, J=8.4 Hz, 1H), 7.65-7.48 (m, 2H), 7.46-7.34 (m, 2H), 7.29 (br s, 1H), 7.18 (br s, 1H), 6.99 (s, 2H), 5.03 (br s, 2H), 4.96 (br s, 1H), 4.72 (br s, 1H), 4.48-4.26 (m, 1H), 4.26-4.04 (m, 1H), 2.22-2.02 (m, 2H), 2.02-1.80 (m, 1H), 1.58-1.37 (m, 4H), 1.36-0.92 (br m, 15H), 0.92-0.53 (br m, 7H). MS (ESI+) m/z 826 (M+1).

The following ATAC30, ATAC31, ATAC32, ATAC33, ATAC34, ATAC35, ATAC36, ATAC37, ATAC38, ATAC39, ATAC40, ATAC41, and ATAC42 are prepared using a method similar to that described above for ATAC29.

### ATAC23: 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate

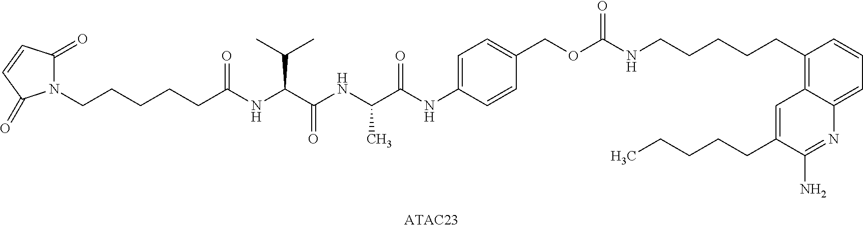 [SMILES: CCCCCc1cc2c(CCCCCNC(=O)OCc3ccc(NC(=O)[C@H](C)NC(=O)[C@@H](NC(=O)CCCCCN4C(=O)C=CC4=O)C(C)C)cc3)cccc2nc1N]

¹H NMR (CD₃OD) δ 8.35 (s, 1H), 7.63 (t, J=8.5 Hz, 1H), 7.55 (d, J=8.0 Hz, 1H), 7.47 (d, J=8.0 Hz, 1H), 7.33 (d, J=8.0 Hz, 1H), 7.27 (d, J=8.0 Hz, 1H), 6.78 (s, 2H), 5.00 (s, 2H), 4.46 (q, J=7.0 Hz, 2H), 4.13 (d, J=7.0 Hz, 1H), 3.47-3.4 (m, 3H), 3.17 (t, J=7.0 Hz, 2H), 3.05 (t, J=7.0 Hz, 2H), 2.75 (t, J=7.5 Hz, 2H), 2.27 (t, J=7.5 Hz, 2H), 2.07 (q, J=7.0 Hz, 1H), 1.72-1.51 (m, 10H), 1.46-1.35 (m, 8H), 1.32-1.26 (m, 3H), 1.00-0.92 (m, 9H). LCMS [M+H]=812.4.

### ATAC24: 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate

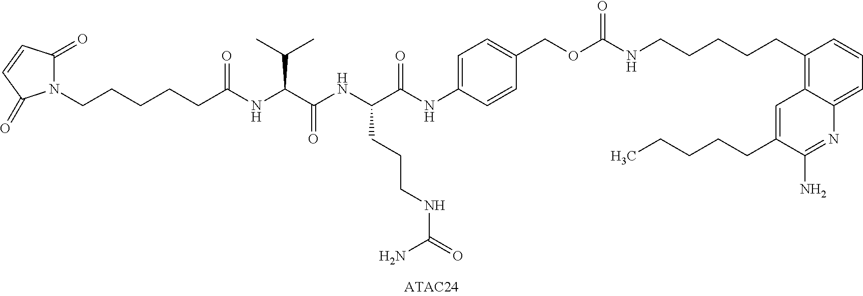 [SMILES: CCCCCc1cc2c(CCCCCNC(=O)OCc3ccc(NC(=O)[C@H](CCCNC(N)=O)NC(=O)[C@@H](NC(=O)CCCCCN4C(=O)C=CC4=O)C(C)C)cc3)cccc2nc1N]

¹H NMR (DMSO-d⁶) δ 13.5 (bs, 1H), 10.0 (s, 1H), 8.40 (m, 3H), 8.07 (d, J=7.5 Hz, 1H), 7.80 (d, J=8.5 Hz, 1H), 7.6-7.5 (m, 4H), 7.35-7.25 (m, 2H), 6.01 (m, 1H), 5.42 (s, 1H), 4.89 (s, 2H), 4.41 (q, J=7.0 Hz, 1H), 4.18 (t, J=7.0 Hz, 1H), 3.10-2.90 (m, 6H), 2.75 (t, J=7.5 Hz, 2H), 2.27 (t, J=7.5 Hz, 2H), 2.07 (q, J=7.0 Hz, 1H), 1.72-1.51 (m, 10H), 1.46-1.35 (m, 8H), 1.32-1.26 (m, 3H), 1.00-0.92 (m, 9H). LCMS [M+H]=898.

### ATAC25: 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt

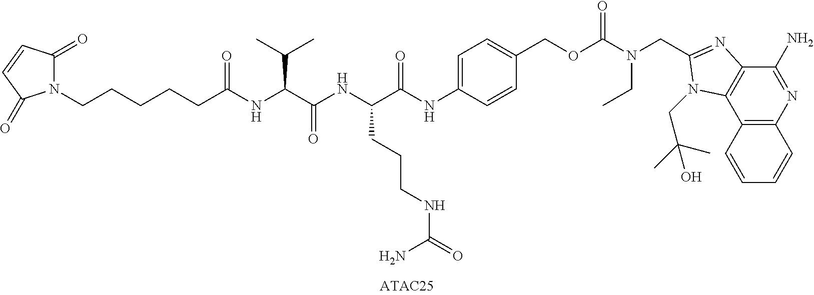 [SMILES: CCN(Cc1nc2c(N)nc3ccccc3c2n1CC(C)(C)O)C(=O)OCc1ccc(NC(=O)[C@H](CCCNC(N)=O)NC(=O)[C@@H](NC(=O)CCCCCN2C(=O)C=CC2=O)C(C)C)cc1]

¹H NMR (DMSO-d⁶) δ 13.4 (bs, 1H), 9.99-9.89 (br m, 1H), 9.09-8.40 (m, 3H), 8.07 (d, J=7.5 Hz, 1H), 7.80 (d, J=8.5 Hz, 1H), 7.68 (t, J=8.0 Hz, 1H), 7.59 (bs, 1H), 7.51 (t, J=8.5 Hz, 1H), 7.46-7.14 (m, 2H), 7.00 (s, 1H), 5.99 (br s, 1H), 5.05 (br s, 1H), 4.95 (br s, 1H), 4.37 (q, J=7.0 Hz, 1H), 4.18 (t, J=7.0 Hz, 1H), 3.37 (t, J=7.0 Hz, 2H), 3.03-2.93 (m, 2H), 2.22-2.07 (m, 2H), 1.99-1.92 (m, 1H), 1.75-1.05 (br m, 20H), 0.85 (d, J=8.5 Hz, 3H), 0.81 (d, J=8.5 Hz, 3H). MS (ESI+) m/z 912.5 (M+1).

TABLE 25

| Compound | Structure | Name | M + 1 |
|---|---|---|---|
| ATAC26 | 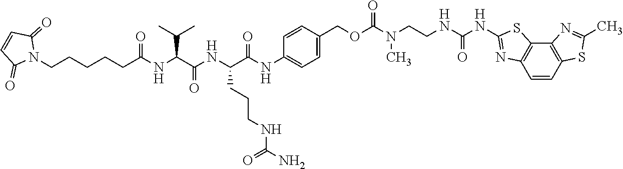 [SMILES: Cc1nc2c(ccc3nc(NC(=O)NCCN(C)C(=O)OCc4ccc(NC(=O)[C@H](CCCNC(N)=O)NC(=O)[C@@H](NC(=O)CCCCCN5C(=O)C=CC5=O)C(C)C)cc4)sc32)s1] | 2-(3-{2-[N-Methyl ({p-[(S)-2-{(S)-2- [6-(2,5-dioxo- 1H-pyrrol-1-yl) hexanoylamino]-3- methylbutyryl- amino}-5- ureidovaleryl- amino]phenyl} methoxycarbonyl) amino]ethyl} | 921 |
| | | ureido)-7- | |
| | | methyl-1,6-dithia- | |
| | | 3,8-diaza-as-indacene | |
| ATAC27 | 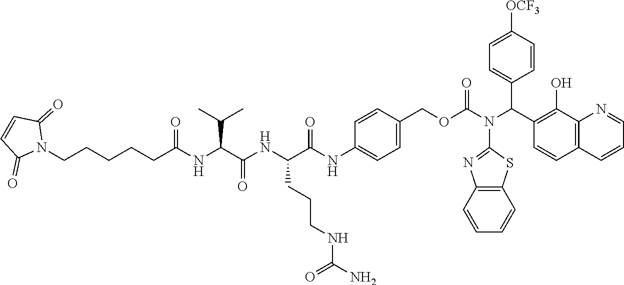 [SMILES: CC(C)[C@H](NC(=O)CCCCCN1C(=O)C=CC1=O)C(=O)N[C@@H](CCCNC(N)=O)C(=O)Nc1ccc(COC(=O)N(c2nc3ccccc3s2)C(c2ccc(OC(F)(F)F)cc2)c2ccc3cccnc3c2O)cc1] | 2-{[(8-Hydroxy- 7-quinolyl)(p- trifluoromethoxy- phenyl)methyl] ({p-[(S)-2-{(S)- 2-[6-(2,5-dioxo- 1H-pyrrol-1- yl)hexanoylamino]- 3-methylbutyryl- amino}-5- ureidovalerylamino] phenyl}methoxy- carbonyl) amino}-1,3- benzothiazole | 1067 |
| ATAC28 | 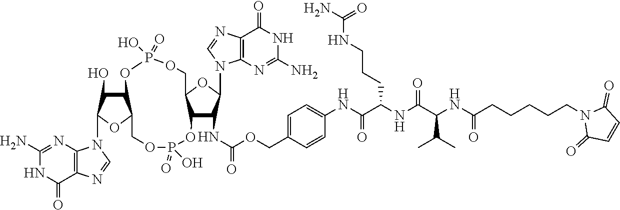 | (1R,6R,8R,9S,10S, 15R,17R,18S)-18- ({p-[(S)-2-{(S)-2-[6- (2,5-Dioxo-1H- pyrrol-1- yl)hexanoylamino]- 3-methylbutyryl- amino}-5- ureidovaleryl- amino]phenyl} methoxycarbonyl amino)-8,17-bis (2-amino-6-oxo- | 1289 |
| | | 1,9-dihydropurin- | |
| | | 9-yl)-3,12- | |
| | | dihydroxy-9-hydroxy- | |
| | | 2.4.7.11.13.16- | |
| | | hexaoxa-3λ5.12λ5- | |
| | | diphosphatricyclo | |
| | | [13.3.0.06,10] | |
| | | octadecane-3,12- | |
| | | dione | |
| ATAC29 | 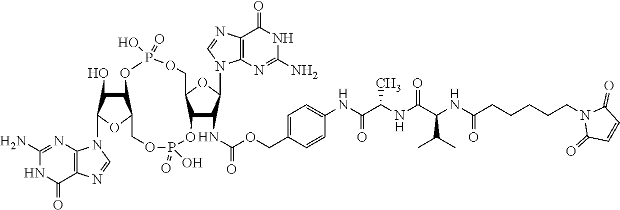 | (1R,6R,8R,9S,10S, 15R,17R,18S)-18- ({p-[(S)-2-{(S)-2-[6- (2,5-Dioxo-1H- pyrrol-1-yl) hexanoylamino]- 3-methylbutyryl- amino}propionyl- amino]phenyl} methoxycarbonyl- amino)-8,17- bis(2-amino-6- oxo-1,9-dihydro- | 1203 |
| | | purin-9-yl)-3,12- | |
| | | dihydroxy-9- | |
| | | hydroxy- | |
| | | 2.4.7.11.13.16- | |
| | | hexaoxa-3λ5.12λ5- | |
| | | diphosphatricyclo | |
| | | [13.3.0.06,10] | |
| | | octadecane-3,12- | |
| | | dione | |
| ATAC30 | 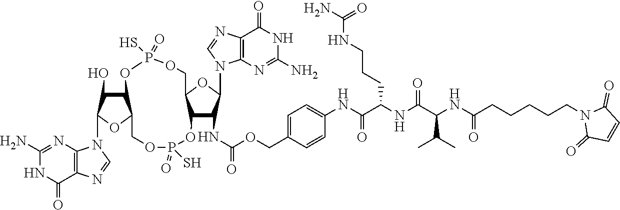 | (1R,6R,8R,9S, 10S,15R,17R,18S)- 18-({p-[(S)- 2-{(S)-2-[6-(2,5- Dioxo-1H-pyrrol-1- yl)hexanoylamino]- 3-methylbutyryl- amino}-5- ureidovalerylamino] phenyl}methoxy- carbonyl amino)-8,17- bis(2-amino-6-oxo- | 1321 |
| | | 1,9-dihydropurin-9- | |
| | | yl)-9-hydroxy-3,12- | |
| | | dimercapto- | |
| | | 2.4.7.11.13.16- | |
| | | hexaoxa-3λ5.12λ5- | |
| | | diphosphatricyclo | |
| | | [13.3.0.06,10] | |
| | | octadecane- | |
| | | 3,12-dione | |
| ATAC31 | 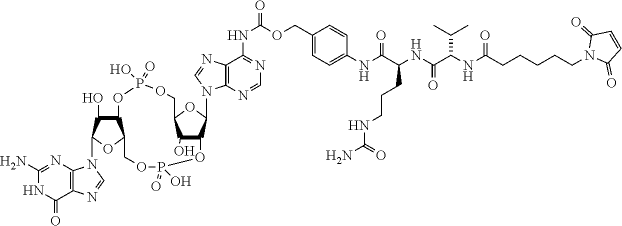 | {p-[(S)-2-{(S)-2- [6-(2,5-Dioxo-1H- pyrrol-1- yl)hexanoylamino]- 3-methylbutyryl- amino}-5- ureidovaleryl- amino]phenyl} methyl 9- {(1S,6R,8R,9S, 10S,15R,17R,18S)- 8-(2-amino-6-oxo- 1,9-dihydropurin- 9-yl)-3,12- | 1274 |
| | | dihydroxy-9,18- | |
| | | dihydroxy-3,12- | |
| | | dioxo- | |
| | | 2.4.7.11.13.16- | |
| | | hexaoxa-3λ5.12λ5- | |
| | | diphosphatricyclo | |
| | | [13.2.1.06,10] | |
| | | octadec-17- | |
| | | yl}-9a-adeninee- | |
| | | carboxylate | |

### Example 9

### Synthesis of ATAC32

This example shows the synthesis of 1-{6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexyl}-1H-pyrrole-2,5-dione (ATAC32).

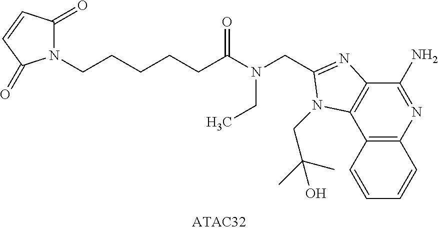 [SMILES: CCN(Cc1nc2c(N)nc3ccccc3c2n1CC(C)(C)O)C(=O)CCCCCN1C(=O)C=CC1=O]

### Step A: Preparation of ATAC32

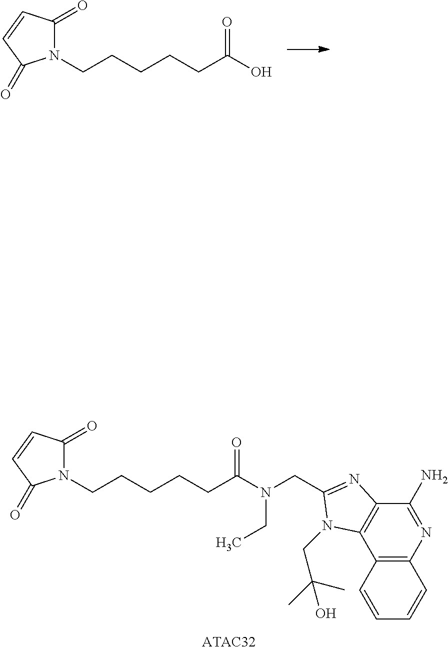 [SMILES: O=C(O)CCCCCN1C(=O)C=CC1=O ; CCN(Cc1nc2c(N)nc3ccccc3c2n1CC(C)(C)O)C(=O)CCCCCN1C(=O)C=CC1=O]

To an ice-cold solution of 6-(2,5-dioxo-2,5-dihydro-1H-pyrrol-1-yl)hexanoic acid (0.034 g, 0.16 mmol) in DCM (0.800 ml) was added 1-chloro-N,N,2-trimethylprop-1-en-1-amine (0.021 mL, 0.160 mmol) dropwise. This was stirred at 0° C. for 1h then added to an ice-cold mixture of compound 1 (50 mg, 0.160 mmol) and triethylamine (66.7 μL, 0.479 mmol) in DCM (800 μL). Overall molarity 0.1 M. The mixture was stirred to room temperature overnight and then chromatographed (DCM to 20% MeOH/DCM) without work-up. Fractions containing product were pooled and evaporated then dissolved in 1 mL of acetonitrile and treated with 0.1 mL of trifluoroacetic acid. The resulting material was evaporated to an oil then redissolved in CH₃CN and lyophilized the sample to give ATAC32 (65 mg) as a white solid. ¹H NMR (400 MHz, (DMSO-d⁶) δ 13.3 (s, 1H), 8.54-8.50 (m, 3H), 7.81 (d, J=8.5 Hz, 1H), 7.76 (d, J=7.5 Hz, 1H), 7.51 (d, J=7.5 Hz, 1H), 6.99 (s, 1H), 6.95 (s, 1H), 3.51 (q, J=7.0 Hz, 2H), 3.43-3.31 (m, 3H), 2.36-2.30 (m, 2H), 1.54-1.41 (m, 4H), 1.25-1.00 (m, 10H). ¹⁹F NMR (DMSO-d⁶) 8-74.0. LCMS [M+H]⁺=507.1.

### Example 10

### Synthesis of ATAC33

This example shows the synthesis of 1-{[4-({6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexylamino}carbonyl)cyclohexyl]methyl}-1H-pyrrole-2,5-dione (ATAC33).

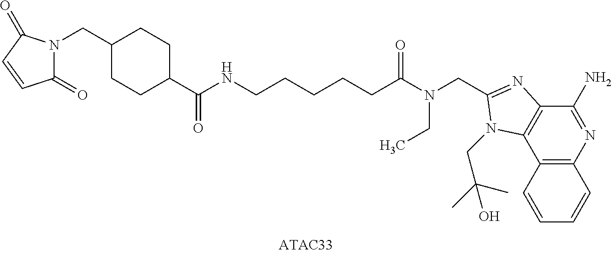 [SMILES: CCN(Cc1nc2c(N)nc3ccccc3c2n1CC(C)(C)O)C(=O)CCCCCNC(=O)C1CCC(CN2C(=O)C=CC2=O)CC1]

### Step A: Preparation of ATAC33

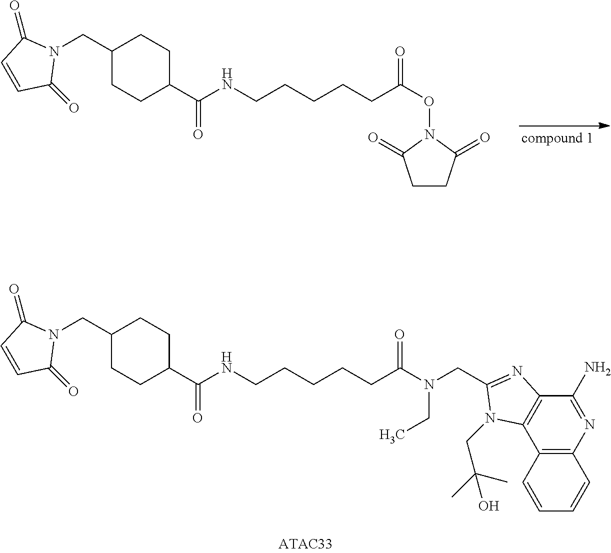 [SMILES: O=C(CCCCCNC(=O)C1CCC(CN2C(=O)C=CC2=O)CC1)ON1C(=O)CCC1=O ; CCN(Cc1nc2c(N)nc3ccccc3c2n1CC(C)(C)O)C(=O)CCCCCNC(=O)C1CCC(CN2C(=O)C=CC2=O)CC1]

To a stirred solution of 1-(4-amino-2-((ethylamino)methyl)-1H-imidazo[4,5-c]quinolin-1-yl)-2-methylpropan-2-ol (100 mg, 0.319 mmol) in DCM (10 mL) under nitrogen was added via a syringe pump a solution of 2,5-dioxopyrrolidin-1-yl 6-(4-((2,5-dioxo-2,5-dihydro-1H-pyrrol-1-yl)methyl)cyclohexane-1-carboxamido)hexanoate (143 mg, 0.319 mmol) in DCM (5 mL) over a period of 3.5 h. The reaction mixture was stirred at room temperature overnight. The reaction mixture was concentrated and the residue was purified by reverse phase column chromatography. Pure fractions identified by HPLC analysis were pooled and concentrated. The residue was lyophilized from CH₃CN to provide a white solid (52.8, mg) as the TFA salt of ATAC33 as a mixture of cis and trans isomers. ¹H NMR (400 MHz, (CD₃OD) δ 8.54 and 8.48 (d, J=8.3 Hz, 1H), 7.81-7.71 (m, 2H), 7.62-7.55 (m, 1H), 6.80 (s, 2H), 3.66 (q, J=7.0 Hz, 2H), 3.13 and 3.08 (t, J=7.0 Hz, 2H), 2.45 and 2.38 (t, J=7.5 Hz, 2H), 2.1-2.0 (m, 1H), 1.8-1.47 (m, 10H), 1.46-1.15 (m, 16H), 1.54-1.41 (m, 4H), 1.25-1.00 (m, 10H). LCMS [M+H]⁺=646.3.

### Example 11

### Synthesis of ATAC34

This example shows the synthesis of 1-[(4-{[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-carbonyl}cyclohexyl)methyl]-1H-pyrrole-2,5-dione (ATAC34).

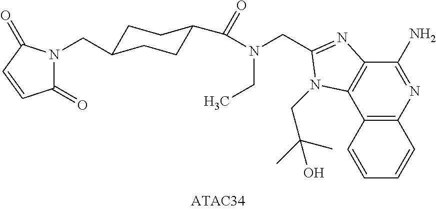 [SMILES: CCN(Cc1nc2c(N)nc3ccccc3c2n1CC(C)(C)O)C(=O)C1CCC(CN2C(=O)C=CC2=O)CC1]

### Step A: Preparation of ATAC34

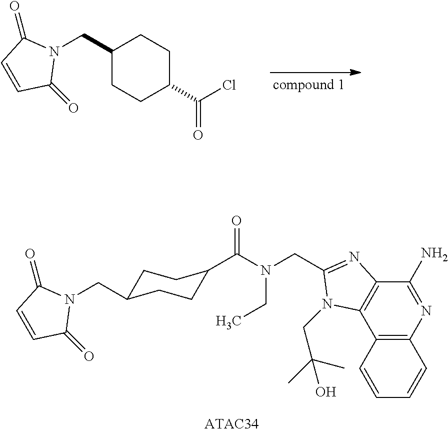 [SMILES: O=C1C=CC(=O)N1C[C@H]1CC[C@H](C(=O)Cl)CC1 ; CCN(Cc1nc2c(N)nc3ccccc3c2n1CC(C)(C)O)C(=O)C1CCC(CN2C(=O)C=CC2=O)CC1]

To an ice-cold solution of (1r,4r)-4-((2,5-dioxo-2,5-dihydro-1H-pyrrol-1-yl)methyl)cyclohexane-1-carboxylic acid (82 mg, 0.346 mmol) in DCM (1728 μl) was added 1-chloro-N,N,2-trimethylprop-1-en-1-amine (50.3 μL, 0.380 mmol) dropwise. This was stirred at 0° C. for 1h then added to an ice-cold mixture of 1-(4-amino-2-((ethylamino)methyl)-1H-imidazo[4,5-c]quinolin-1-yl)-2-methylpropan-2-ol (100 mg, 0.319 mmol) and triethylamine (133 μl, 0.957 mmol) in 1.6 mL of DCM. The mixture became a yellow solution as it stirred overnight to room temp. The reaction was concentrated to dryness, redissolved in MeOH/CH₂Cl₂, silica gel was added, then the solvents evaporated. Chromatography (12 g Gold silica, DCM to 20% MeOH/DCM, dry load) gave a solid which was dissolved in CH₃CN, frozen and lyophilized to afford 170 mg of N-((4-((1-(dimethylamino)-2-methylprop-1-en-1-yl)amino)-1-(2-((1-(dimethylamino)-2-methyl-prop-1-en-1-yl)oxy)-2-methylpropyl)-1H-imidazo[4,5-c]quinolin-2-yl)methyl)-4-((2,5-dioxo-2,5-dihydro-1H-pyrrol-1-yl)methyl)-N-ethylcyclohexane-1-carboxamide which was subsequently dissolved in 50% aqueous MeCN containing 0. I1% TFA and heated in a microwave reactor at 150° C. for 60 min. The reaction mixture was cooled and the solvents were evaporated and chromatographed to give ATAC34 (72 mg) as a white solid. ¹H NMR (400 MHz, (DMSO-d⁶) δ 13.3 (s, 1H), 8.70-8.50 (m, 3H), 7.83-7.79 (m, 1H), 7.71-7.65 (m, 1H), 7.55-7.48 (m, 1H), 7.00 (s, 1H), 6.98 (s, 1H), 5.13 (bs, 1H), 4.83 (bs, 1H), 3.65 (q, J=7.0 Hz, 2H), 3.38 (m, 1H), 3.25 and 3.18 (d, J=6.5 Hz, 2H), 1.69-1.52 (m, 5H), 1.45-0.88 (m, 13H). ¹⁹F NMR (DMSO-d⁶) 8-73.7. LCMS [M+H]⁺=533.1.

### Example 12

### Determination of Kd Values

K_{d} is measured by a radiolabeled antigen binding assay (RIA) performed with the Fab version, bispecific antibody construct version, or the conjugate version comprising the antigen binding domain of interest, and its antigen as described by the following assay.

Solution binding affinity of Fabs, bispecific antibody constructs, or conjugates comprising the antigen binding domain for the antigen of interest is measured by equilibrating the Fab, bispecific antibody constructs, or conjugate with a minimal concentration of (¹²⁵I)-labeled antigen in the presence of a titration series of unlabeled antigen, then capturing bound antigen with an anti-Fab, anti-bispecific antibody construct, or anti-conjugate antibody-coated plate (See, e.g., Chen et al., J. Mol. Biol. 293:865-881 (1999)). To establish conditions for the assay, multi-well plates are coated overnight with 5 μg/mL of a capturing anti-Fab, anti-bispecific antibody construct, or anti-conjugate antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab, bispecific antibody construct, or conjugate of interest (e.g., consistent with assessment of the anti-VEGF antibody, Fab-12, in Presta et al., Cancer Res. 57:4593-4599 (1997)). The Fab, bispecific antibody construct, or conjugate of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant is added, and the plates are counted on a TOPCOUNT™ gamma counter (Packard) for ten minutes. Concentrations of each Fab, bispecific antibody construct, or conjugate that give less than or equal to 20% of maximal binding are chosen for use in competitive binding assays.

### Example 13

### Determination of Kd Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab, bispecific antibody construct, or conjugate (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form, bispecific form, or conjugate form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 14

### Lysine-Based Bioconjugation

The antibody was exchanged into an appropriate buffer, for example, phosphate, borate, PBS, or Tris-Acetate, at a concentration of about 2 mg/mL to about 10 mg/mL. An appropriate number of equivalents of the immune stimulatory compound-linker construct (ATAC) were added as a solution with stirring. Dependent on the physical properties of the immune stimulatory compound-linker construct, a co-solvent was introduced prior to the addition of the immune stimulatory compound-linker construct to facilitate solubility. The reaction was stirred at room temperature for 2 hours to about 12 hours depending on the observed reactivity. The progression of the reaction was monitored by LC-MS. Once the reaction was deemed complete, the remaining immune stimulatory compound-linker constructs were removed by applicable methods and the lysine-linked immune-stimulatory conjugate was exchanged into the desired formulation buffer.

Lysine-linked immune-stimulatory conjugates were synthesized starting with 10 mg of antibody (mAb) and 10 equivalents of ATAC1, ATAC2, ATAC3, ATAC4, ATAC5, ATAC6, ATAC7, ATAC8, ATAC9, or ATAC10 using the conditions described in Scheme 34 below (ADC=antibody immune-stimulatory compound conjugate; immune-stimulatory conjugate).

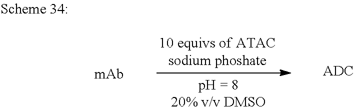

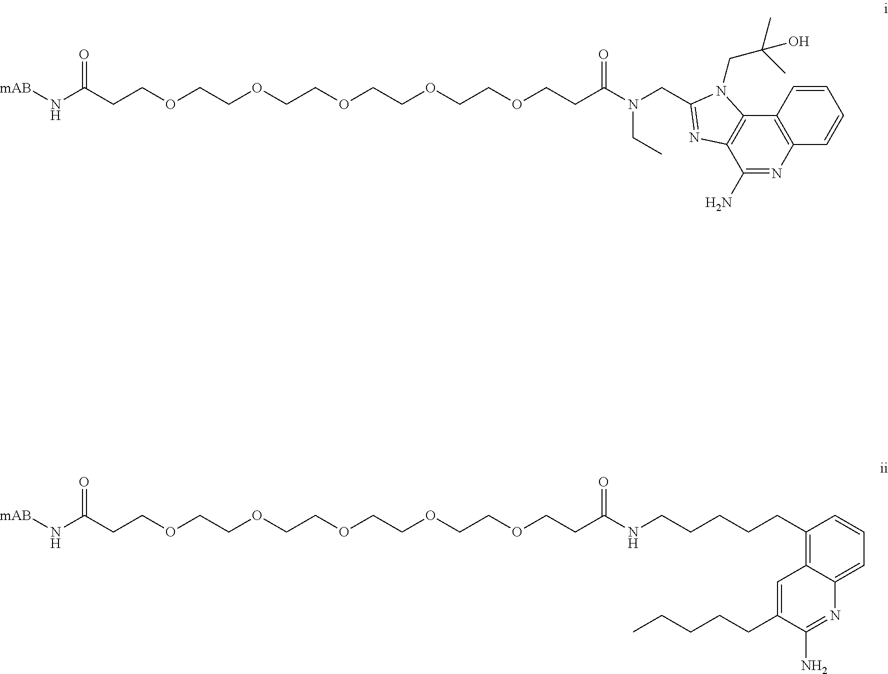

### Example 15

### Cysteine-Based Bioconjugation

The antibody was exchanged into an appropriate buffer, for example, phosphate, borate, PBS, or Tris-Acetate, at a concentration of about 2 mg/mL to about 10 mg/mL with an appropriate number of equivalents of a reducing agent, for example, dithiothreitol or tris(2-carboxyethyl)phosphine. The resultant solution was stirred for an appropriate amount of time and temperature to effect the desired reduction. The immune stimulatory compound-linker construct was added as a solution with stirring. Dependent on the physical properties of the immune stimulatory compound-linker construct, a co-solvent was introduced prior to the addition of the immune stimulatory compound-linker construct to facilitate solubility. The reaction was stirred at room temperature for about 1 hour to about 12 hours depending on the observed reactivity. The progression of the reaction was monitored by liquid chromatography-mass spectrometry (LC-MS). Once the reaction was deemed complete, the remaining free immune stimulatory compound-linker construct was removed by applicable methods and the conjugate was exchanged into the desired formulation buffer. Such cysteine-based conjugates can be synthesized starting with 10 mg of antibody (mAb) and 7 equivalents of ATAC11-ATAC34 using the conditions described in Scheme 35 below (ADC=antibody immune-stimulatory compound conjugates; immune-stimulatory conjugates). Monomer content and drug-antibody ratios can be determined by methods described in EXAMPLES 16-17.

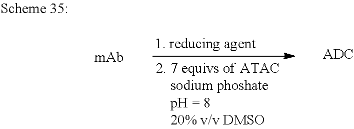

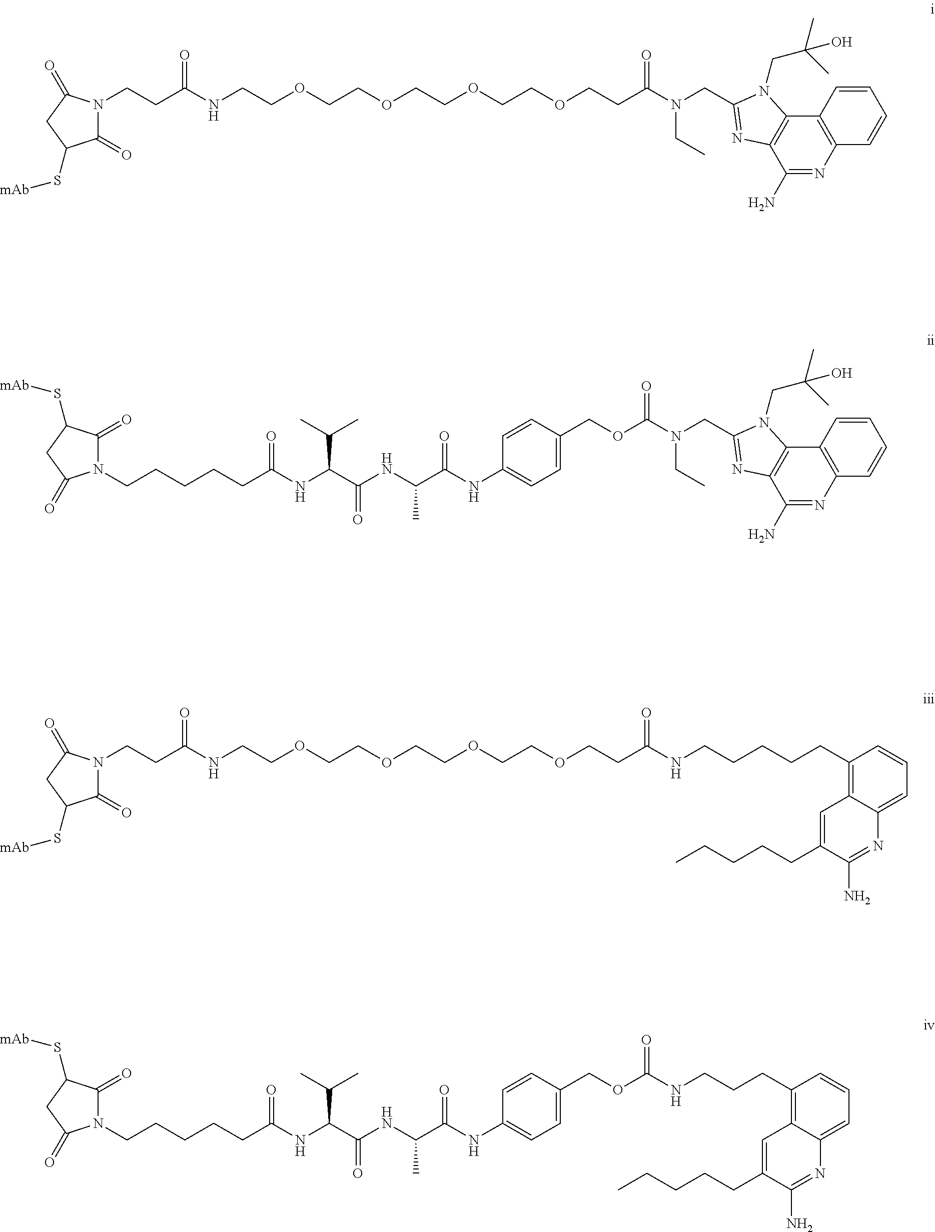

### Example 16

### Determination of Molar Ratio

This example illustrates one method by which the molar ratio is determined. One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which can use top peak height at 25% for the calculation. This data is then imported into another program to calculate the molar ratio of the immune-stimulatory compound:conjugate, such as Agilent molar ratio calculator.

### Example 17

### Additional Method for Determination of Molar Ratio

Another method for determination of molar ratio is as follows. First, 10 μL of a 5 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 18

### Generation of a Bispecific Anti-HER2×Anti-CD40 Recombinant Antibody Construct

The anti-CD40 single chain Fv (scFv) in the VH-VL orientation is fused to the C-terminus of the anti-HER2 heavy chain (VH—CH₁—CH₂—CH₃) (pertuzumab heavy chain) to generate the anti-HER2 VH—CH₁—CH₂—CH₃-anti-CD40 scFv heavy chain. This heavy chain is co-expressed with the light chain of anti-HER2 (VL-Ck) (pertuzumab light chain) in the transient CHO system in 180 mL of CHO media. The supernatant is harvested 7 days after the transfection and the bispecific antibody construct is purified over HiScreen MabSelect SuRe Protein A column on GE AKTA Pure machine. The bispecific anti-HER2×anti-CD40 antibody construct is recovered.

### Example 19

### Bispecific Anti-HER2×Anti-CD40 Recombinant Antibody Construct Binds to Human HER2 and Human CD40 Extracellular Domain with Similar Affinity as Parental Anti-HER2 Antibody and Parental Anti-CD40 Antibody

This example shows that the bispecific anti-HER2×anti-CD40 antibody construct binds to human HER2 and human CD40 extracellular domain (ECD).

The bispecific antibody constructs consisting of anti-HER2×anti-CD40 heavy chain and anti-HER2 light chain are co-expressed in the transient CHO system. The bispecific antibody was prepared as described in Example 18. The supernatant is harvested 7 days after transfection and the bispecific antibody construct was purified over HiScreen MabSelect SuRe Protein A column on GE AKTA Pure machine. Protein is analyzed for purity on Size Exclusion Chromatography using Agilent 1260 Infinity machine. Protein is recovered from 60 mL CHO transfection and is used for binding analysis. Analysis of human HER2 and CD40 ECD interactions are performed using Octet Red 96 instrument (ForteBio). The Octet systems use propriety BLI to analyze biomolecular interaction. Anti-HER2 parental antibody, anti-CD40 parental antibody and bispecific recombinant antibody construct are immobilized on anti-human Fc biosensors and incubated with varying concentration of monomeric human HER2 and human CD40 ECD ranging from 1.2 nM to 300 nM in PBS/0.1% BSA/0.02% Tween 20. The studies comprise 5 steps: (1) baseline acquisition (60 s); (2) loading of parental antibodies and bispecific recombinant antibody constructs onto an anti-human Fc biosensor (120 s); (3) second baseline acquisition (60 s); (4) association of interacting monomeric HER2 and CD40 ECD proteins for kₒₙ measurement (120 s); and (5) dissociation of interacting monomeric HER2 and CD40 ECD for k_{off} measurement (300 s). The interacting monomeric HER2 and CD40 ECDs are used at 6 concentrations of 3-fold concentration series. Data is analyzed using Octet Data Analysis Software 9.0 (ForteBio) and fitted to the 1:1 binding model. Equilibrium dissociation constants (K_{D}) are calculated by the ratio of kₒₙ to k_{off}. The binding of the bispecific anti-HER2×anti-CD40 antibody construct to HER2 and CD40 ECD are similar to the binding of the parental anti-HER2 antibody and anti-CD40 antibody.

### Example 20

### Bispecific Anti-HER2×Anti-CD40 Recombinant Antibody Construct Binds to Fcγ Receptors

This example shows that the bispecific anti-HER2×anti-CD40 antibody construct binds to human Fcγ receptors (FcγRs). The anti-HER2×anti-CD40 antibody was prepared as described in Example 18. Human FcγR interaction analysis was performed using an Octet Red 96 Instrument™. For human FcγRI and FcγRIIA interactions, the recombinant bispecific antibody was immobilized on anti-human Fc biosensors and incubated with varying concentration of monomeric FcγR ranging from 1.2 nM to 300 nM in PBS/0.1% BSA/0.02% Tween 20. The experiments were performed using five steps: (1) baseline acquisition (60 s); (2) bispecific construct loading onto anti-human Fc biosensor (120 s); (3) second baseline acquisition (60 s); (4) association of interacting FcγR for kₒₙ measurement (120 s); and (5) dissociation of interacting FcγR for k_{off} measurement (300 s). The interacting monomeric FcγRs are used at 4-6 concentrations of a 3-fold concentration series. The data were analyzed using Octet Data Analysis Software 9.0 (ForteBio)™ and fit to the 1:1 binding model. Equilibrium dissociation constants (K_{D}) are calculated by the ratio of kₒₙ to k_{off}.

For human FcγRIIIA F158 and FcγRIIIA V158 interaction studies, the FcγRs were immobilized on anti-His tag biosensors and incubated with varying concentrations of bispecific antibody ranging from 37 nM to 1 μM. The studies were performed using five steps: (1) Baseline acquisition (30 s); (2) human FcγRs loading to the anti-His tag biosensor (90 s); (3) second baseline acquisition (30 s); (4) association of interacting bispecific constructs for kₒₙ measurement (30 s); and (5) dissociation of interaction bispecific antibody constructs for k_{off} measurement (30 s). The interacting bispecific antibody constructs are used at 4 concentrations of a 3-fold concentration series. The data was analyzed using Octet Data Analysis Software 9.0 (ForteBio)™ and fit to the avidity binding model. Equilibrium dissociation constants (K_{D}) are calculated by the ratio of kₒₙ to k_{off}. The data are shown in TABLE 26 along with data for the control monospecific antibodies tested using the same protocol as just described. The bispecific anti-HER2×anti-CD40 antibody construct bound to the Fcγ receptors similarly to the antibody construct comprising the anti-HER2 IgG antibody.

TABLE 26
Binding affinity of recombinant antibody constructs targeting Her2/Neu and CD40

| | Hu CD40 | | Hu Her2 | huFcγR1 | huFcγR2A | huFcγR3 | huFcγR3 |
|---|---|---|---|---|---|---|---|
| | K_{D} | | K_{D} | K_{D} | K_{D} | F158 K_{D} | V158 K_{D} |
| Parental | 8.4 | nM | No binding | 0.7 nM | 27 nM | 0.86 uM | 0.51 uM |
| anti- | | | | | | | |
| CD40 | | | | | | | |
| antibody | | | | | | | |
| Parental | No binding | | 0.9 nM | 1.0 nM | 19 nM | 0.83 uM | 0.38 uM |
| anti- | | | | | | | |
| HER2 | | | | | | | |
| antibody | | | | | | | |
| Anti- | 13.3 | nM | 1.3 nM | 1.2 nM | 36 nM | 0.88 uM | 0.30 uM |

HER2 X
anti-
CD40 Fc
IgG1

Legend: nM = nanomolar

### Example 21

### Bispecific Anti-HER2×Anti-DEC205 IgG1 Antibody Construct Binds to Human HER2 and Human DEC205 Extracellular Domain with Similar Affinity as Parental Anti-HER2 Antibody and Parental Anti-DEC205 Antibody

This example shows that the anti-HER2×anti-DEC205 IgG1 bispecific antibody construct binds to human HER2 extracellular domain (ECD) and human DEC205 extracellular domain (ECD).

The bispecific antibody construct consisting of anti-HER2×anti-DEC205 IgG1 heavy chain and anti-HER2 light chain were co-expressed in the transient CHO system. The heavy chain comprised pertuzumab heavy chain having a DEC205 scFV attached at the C-terminal end. The light chain of pertuzumab was also used. The supernatant was harvested 7 days after transfection and the bispecific antibody construct was purified over HiScreen MabSelect SuRe Protein A column on GE AKTA Pure machine. Protein was analyzed for purity on Size Exclusion Chromatography using Agilent 1260 Infinity machine. 10.87 mg of protein was recovered from 60 mL CHO transfection and was used for binding analysis. Analysis of human HER2 and DEC205 ECD interactions were performed using Octet Red 96 instrument (ForteBio). The Octet systems use propriety BLI to analyze biomolecular interaction. Anti-HER2 parental antibody, anti-DEC205 parental antibody, and bispecific antibody constructs were immobilized on anti-human Fc biosensors and incubated with varying concentration of monomeric human HER2 ECD or human DEC205 ECD ranging from 1.2 nM to 300 nM in PBS/0.1% BSA/0.02% Tween 20. The studies were comprised of 5 steps: (1) baseline acquisition (60 s); (2) parental antibodies and bispecific antibody construct loading onto anti-human Fc biosensor (120 s); (3) second baseline acquisition (60 s); (4) association of interacting monomeric HER2 ECD and DEC205 ECD proteins for kₒₙ measurement (120 s); and (5) dissociation of interacting monomeric HER2 ECD and DEC205 ECD for k_{off} measurement (480-900 s). The interacting monomeric HER2 and DEC205 ECDs were used at 6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. The anti-HER2×anti-DEC205 bispecific antibody constructs demonstrated similar binding as the parental anti-HER2 antibody and weaker but sufficient binding as the parental anti-DEC205 as shown below in TABLE 27.

TABLE 27

| | Human HER2 | Human DEC205 |
|---|---|---|
| | K_{D} (nM) | K_{D} (nM) |
| Parental Anti-HER2 Antibody | 0.94 | No Binding |
| Parental Anti-DEC205 | No Binding | 0.85 |
| Antibody | | |
| Anti-HER2 × Anti-DEC205 | 0.72 | 12.2 |
| IgG1 Bispecific antibody | | |
| construct | | |

### Example 22

### Bispecific Anti-CEA×Anti-CD40 IgG1 Antibody Construct Binds to Human CEA and Human CD40 Extracellular Domain with Similar Affinity as Parental Anti-CEA Antibody and Parental Anti-CD40 Antibody

This example shows that the anti-CEA×anti-CD40 IgG1 bispecific antibody construct binds to human CEA extracellular domain (ECD) and human CD40 extracellular domain (ECD).

The bispecific antibody construct consisting of anti-CEA×anti-CD40 IgG1 heavy chain (prepared by attaching a CD40 scFC to the C terminus of the anti-CEA heavy chain) and anti-CEA light chain were co-expressed in the transient CHO system. The supernatant was harvested 7 days after transfection and the bispecific antibody construct was purified over HiScreen MabSelect SuRe Protein A column on GE AKTA Pure machine. Protein was analyzed for purity on Size Exclusion Chromatography using Agilent 1260 Infinity machine. 4.45 mg of protein was recovered from 30 mL CHO transfection and was used for binding analysis. Analysis of human CEA ECD or human CD40 ECD interactions were performed using Octet Red 96 instrument (ForteBio). The Octet systems use propriety BLI to analyze biomolecular interaction. Anti-CEA parental antibody, anti-CD40 parental antibody, and bispecific antibody constructs were immobilized on anti-human Fc biosensors and incubated with varying concentration of monomeric human CEA ECD or human CD40 ECD ranging from 1.2 nM to 300 nM in PBS/0.1% BSA/0.02% Tween 20. The studies were comprised of 5 steps: (1) baseline acquisition (60 s); (2) parental antibodies and bispecific antibody construct loading onto anti-human Fc biosensor (120 s); (3) second baseline acquisition (60 s); (4) association of interacting monomeric CEA ECD or CD40 ECD proteins for kₒₙ measurement (120 s); and (5) dissociation of interacting monomeric CEA ECD or CD40 ECD for k_{off} measurement (480-900 s). The interacting monomeric CEA ECDs or CD40 ECDs were used at 6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. The anti-CEA×anti-CD40 bispecific antibody construct demonstrated similar binding as the parental anti-CEA antibody and as the parental anti-CD40 antibody as shown below in TABLE 28.

TABLE 28

| | Human | |
|---|---|---|
| | CEA K_{D} (nM) | Human CD40 K_{D} (nM) |
| Parental Anti-CEA Antibody | 18.5 | No Binding |
| Parental Anti-CD40 Antibody | No Binding | 8.4 |
| Anti-CEA × anti-CD40 IgG1 | 23.7 | 10.0 |
| Bispecific antibody construct | | |

### Example 23

### Antibody Conjugates, Bispecific Antibody Constructs and Bispecific Conjugates Bind to Fcγ Receptors with Similar Affinity as Parental Controls

This example shows that antibody conjugates, bispecific mAbs and bispecific conjugates bind to Fcγ receptors with similar affinity as the parental controls. The analyses of Her2, Trop2, CEACAM5 (CEA), PD-L1, and ROR1 extracellular domain (ECD) interactions were performed using Octet Red 96 instrument (ForteBio). The Octet system uses propriety BLI to analyze biomolecular interactions.

The anti-HER2×anti-CD40 antibody was prepared as described in Example 18. Other bispecific antibodies were prepared by a similar procedure (attaching a CD40 scFc to the C terminal end of the heavy chain. Antibody conjugates and bispecific antibody conjugates were prepared by standard conjugation procedures, as described for example in Example 15. Unconjugated mono-/bispecific antibodies and antibody immune-stimulatory compound conjugates were immobilized on anti-human Fc biosensors and incubated with varying concentrations of monomeric human ECDs (Her2, Trop2, CEACAM5 (CEA), PD-L1, ROR1) ranging from 1.2 nM to 300 nM in PBS/1% BSA/0.2% Tween 20. The studies comprised 5 steps: (1) baseline acquisition (30 s); (2) unconjugated mono-/bispecific antibodies and antibody immune-stimulatory compound conjugates loading onto anti-human Fc biosensor (120 s); (3) second baseline acquisition (30 s); (4) association of interacting monomeric ECD proteins for kₒₙ measurement (120 s); and (5) dissociation of interacting monomeric ECDs for k_{off} measurement (240 s). The interacting monomeric ECDs were used at 5-6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. Data are shown in TABLE 29. The antibody immune-stimulatory compound conjugates had similar binding affinity (Kd) as unconjugated mono-/bispecific antibodies to monomeric human ECDs.

Human FcγR interaction analyses were also performed using Octet Red 96 instrument. For human FcγRI and FcγRIIA interactions, unconjugated mono-/bispecific antibodies or antibody immune-stimulatory compound conjugates were immobilized on anti-human Fc biosensors and incubated with varying concentrations of monomeric FcγRI or FcγRIIA protein ranging from 1.2 nM to 300 nM in PBS/1% BSA/0.2% Tween 20. The studies comprised 5 steps: (1) baseline acquisition (45 s); (2) mono-/bispecific antibodies and antibody immune-stimulatory compound conjugates loading onto anti-human Fc biosensor (120 s); (3) second baseline acquisition (45 s); (4) association of interacting FcγRs for kₒₙ measurement (90-120 s); and (5) dissociation of interacting FcγRs for k_{off} measurement (90-120 s). The interacting monomeric FcγRI and FcγRIIA proteins were used at 4-6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. Data are shown in TABLE 29. There were little to no changes in different antibody immune-stimulatory compound conjugate interactions with human FcγRI and FcγRIIA as compared to unconjugated mono-/bispecific antibody interactions with the respective FcγR monomeric proteins.

For human FcγRIIIA V176 interaction studies, the FcγRIIIA V176 protein was immobilized on anti-His tag biosensors and incubated with varying concentration of unconjugated mono-/bispecific antibodies or antibody immune-stimulatory compound conjugates ranging from 0.04 μM to 1 μM. This format was chosen because of weak interactions if antibodies were captured first and FcγRIIIA V176 protein added afterwards. The study comprised 5 steps: (1) baseline acquisition (45 s); (2) unconjugated mono-/bispecific antibodies or antibody immune-stimulatory compound conjugates loading onto anti-His tag biosensor (120 s); (3) second baseline acquisition (45 s); (4) association of interacting antibodies and antibody immune-stimulatory compound conjugates for kₒₙ measurement (60 s); and (5) dissociation of interacting antibodies and antibody immune-stimulatory compound conjugates for k_{off} measurement (60 s). The unconjugated mono-/bispecific antibodies or antibody immune-stimulatory compound conjugates were used at 4 concentrations of a 3-fold concentration series. Data were analyzed using Octet Data Analysis Software 9.0 (ForteBio) and fitted to the 1-1 binding model. Equilibrium dissociation constants (K_{D}) were calculated by the ratio of kₒₙ to k_{off}. Data are shown in TABLE 29. There were no changes in binding of the antibody immune-stimulatory compound conjugates with human FcγRIIIA V176 protein when compared to the parental unconjugated antibodies.

TABLE 29

| | | | huFcyRI | huFcyRIIA | huFcyRIIIA V176 |
|---|---|---|---|---|---|
| Antibody/Conjugate | huECDs (nM) | | (1-1, nM) | (1-1, nM) | (avidity, uM) |
| Parental anti-Her2 mAb | 1.4 | (Her2) | 1.1 | 11.1 | 0.26 |
| Anti-Her2 ATAC conjugate 1 | 1.7 | (Her2) | 1.3 | 17.8 | 0.37 |
| Anti-Her2 ATAC conjugate 2 | 1.6 | (Her2) | 1.5 | 14.3 | 0.33 |
| Anti-Her2 ATAC conjugate 3 | 1.9 | (Her2) | 1.3 | 9.1 | 0.34 |
| Her2 × CD40 bispecific | 1.9 | (Her2) | 1.2 | 26.9 | 0.22 |
| | 10.7 | (CD40) | | | |
| Her2 × CD40-ATAC conjugate 1 | 2.0 | (Her2) | 1.3 | 19.4 | 0.19 |
| | 9.5 | (CD40) | | | |
| Her2 × CD40-ATAC conjugate 2 | 2.0 | (Her2) | 1.9 | 14.5 | 0.14 |
| | 8.1 | (CD40) | | | |
| Parental anti-TROP2 mAb | 3.1 | (Trop2) | 1.6 | 12.0 | 0.45 |
| Anti-TROP2 ATAC conjugate 1 | 2.4 | (Trop2) | 2.3 | 19.4 | 0.47 |
| Anti-TROP2 ATAC conjugate 2 | 1.3 | (Trop2) | 1.8 | 16.1 | 0.39 |
| Anti-TROP2 ATAC conjugate 3 | 5.3 | (Trop2) | 1.6 | 26.3 | 0.54 |
| Trop2 × CD40 bispecific | 1.5 | (Trop2) | 0.9 | 15.0 | 0.21 |
| | 7.3 | (CD40) | | | |
| Trop2 × PDL1 bispecific | 2.1 | (Trop2) | 1.5 | 9.3 | 0.27 |
| | 3.5 | (PD-L1) | | | |
| Parental anti-CEACAM5 mAb | 1.9 | (CAM5) | 1.6 | 20.5 | 0.33 |
| Anti-CEACAM5 conjugate 4 | 2.5 | (CAM5) | 1.5 | 26.4 | 0.45 |
| Anti-CEACAM5 conjugate 5 | 2.5 | (CAM5) | 1.7 | 16.7 | 0.43 |
| Anti-PD-L1 mAb | 2.5 | (PD-L1) | 1.3 | 15.4 | 0.24 |
| Anti-PD-L1 ATAC conjugate 6 | 3.3 | (PD-L1) | 1.2 | 21.4 | 0.39 |
| Anti-ROR1 mAb | 2.6 | (ROR1) | 1.1 | 20.8 | 0.19 |
| ROR1 × CD40 bispecific | 2.4 | (ROR1) | 1.2 | 10.0 | 0.23 |
| | 8.2 | (CD40) | | | |

### Example 24 Dendritic Cell Cytokine Release by Bispecific Tumor Targeting Conjugate when the Conjugate is Bound to Tumor Antigen

This example shows that the immune stimulatory activity of a bispecific tumor targeting conjugate is retained when bound to a tumor antigen. Myeloid dendritic cells (mDCs) are derived from CD14⁺ monocytes isolated from human peripheral blood mononuclear cells (PBMCs) by negative selection using a commercially available kit. The monocytes are cultured in RPMI containing 10% fetal calf serum for seven days in complete medium supplemented with 25 ng/mL IL-4 and 10 ng/mL GM-CSF. The media is replaced with fresh media plus cytokines on day three. On day six, 5×10⁴ dendritic cells are added in the growth media to separate wells of a 96 well microtiter plate previously coated with varying concentrations of commercially available recombinant soluble HER2 ectodomain, a soluble CEA ectodomain, or a soluble MSLN ectodomain. The coating is performed by incubation of the added soluble tumor antigen at 1 ug/ml for 1 hour at room temperature, followed by 3 washes to remove any unbound protein. A CEA-CD40G1 antibody construct, CEA-DEC205G1 antibody construct, or CEA-CLEC9AG1 antibody construct is added to a well coated with CEA antigen comprising mDCs. A CEA-CD40G1-TLR8 agonist conjugate, CEA-DEC205G1-TLR8 agonist conjugate, or CEA-CLEC9AG1-TLR8 agonist conjugate is added to a well coated with CEA antigen comprising mDCs. A CEA-CD40G1-STING agonist conjugate, CEA-DEC205G1-STING agonist conjugate, or CEA-CLEC9AG1-STING agonist conjugate is added to a well coated with CEA antigen comprising mDCs. A MSLN-CD40G1 conjugate, MSLN-DEC205G1 conjugate, or MSLN-CLEC9AG1 conjugate is added to a well coated with MSLN antigen comprising mDCs. A MSLN-CD40G1-TLR8 agonist conjugate, MSLN-DEC205G1-TLR8 agonist conjugate, or MSLN-CLEC9AG1-TLR8 agonist conjugate is added to a well coated with MSLN antigen comprising mDCs. A MSLN-CD40G1-STING agonist conjugate, MSLN-DEC205G1-STING agonist conjugate, or MSLN-CLEC9AG1-STING agonist conjugate is added to a well coated with MSLN antigen comprising mDCs. A HER2-CD40G1 conjugate, HER2-DEC205G1 conjugate, or HER2-CLEC9AG1 conjugate is added to a well coated with HER2 antigen comprising mDCs. A HER2-CD40G1-TLR8 agonist conjugate, HER2-DEC205G1-TLR8 agonist conjugate, or HER2-CLEC9AG1-TLR8 agonist conjugate is added to a well coated with HER2 antigen comprising mDCs. A HER2-CD40G1-STING agonist conjugate, HER2-DEC205G1-STING agonist conjugate, or HER2-CLEC9AG1-STING agonist conjugate is added to a well coated with HER2 antigen comprising mDCs. Each antibody construct or conjugate is added to a different well at various concentrations. As controls, monospecific tumor antigen antibody is added to wells coated with tumor antigen or the bispecific tumor targeting conjugates described in this example are added to wells coated with an irrelevant tumor antigen. After 24 hour incubation in mDC growth media at 37° C. with a 5% CO₂ atmosphere supernatants are collected and the cytokines IL-6, TNFα, IL-12 and IL-10 that are produced by the mDC are quantitated by electrochemiluminesence signal by multiplex ELISA using commercially available reagents and plate reader from Meso Scale Discovery. When the bispecific tumor targeting conjugates are bound to tumor antigen, the cytokine is produced by mDCs in equal or greater amounts than the cytokine produced from mDCs in wells comprising the bispecific tumor targeting conjugates and an irrelevant tumor antigen or wells comprising monospecific tumor antigen antibody and tumor antigen.

### Example 25

### Cell Surface Immunomodulatory Molecule Expression is Increased by Bispecific Recombinant Antibody Conjugates when the Bispecific Recombinant Antibody Conjugate is Bound to Tumor Antigen

This example shows that human dendritic cell (DC) expression of CD54, CD80, CD86, MHC Class II (HLA-DR), and CD83 is increased when the bispecific recombinant antibody targeting conjugate is bound to tumor antigen, and that this increase is dose dependent. Human PBMCs are isolated. DCs are derived from human PBMCs by isolation of CD14⁺ monocytes followed by culture in RPMI containing 10% fetal calf serum for seven days in complete medium supplemented with 10 ng/mL IL-4 and 100 ng/mL GM-CSF. After three days of culture the media is removed and replaced with fresh media including cytokine supplement. On day six, the following bispecific recombinant antibody constructs or conjugates are added at varying concentrations: a CEA-CD40G1 antibody construct, CEA-DEC205G1 antibody construct, or CEA-CLEC9AG1 antibody construct is added to a well coated with CEA antigen comprising mDCs; a CEA-CD40G1-TLR8 agonist conjugate, CEA-DEC205G1-TLR8 agonist conjugate, or CEA-CLEC9AG1-TLR8 agonist conjugate is added to a well coated with CEA antigen comprising mDCs; a CEA-CD40G1-STING agonist conjugate, CEA-DEC205G1-STING agonist conjugate, or CEA-CLEC9AG1-STING agonist conjugate is added to a well coated with CEA antigen comprising mDCs; a MSLN-CD40G1 antibody construct, MSLN-DEC205G1 antibody construct, or MSLN-CLEC9AG1 antibody construct is added to a well coated with MSLN antigen comprising mDCs; a MSLN-CD40G1-TLR8 agonist conjugate, MSLN-DEC205G1-TLR8 agonist conjugate, or MSLN-CLEC9AG1-TLR8 agonist conjugate is added to a well coated with MSLN antigen comprising mDCs; a MSLN-CD40G1-STING agonist conjugate, MSLN-DEC205G1-STING agonist conjugate, or MSLN-CLEC9AG1-STING agonist conjugate is added to a well coated with MSLN antigen comprising mDCs; a HER2-CD40G1 antibody construct, HER2-DEC205G1 antibody construct, or HER2-CLEC9AG1 antibody construct is added to a well coated with HER2 antigen comprising mDCs; a HER2-CD40G1-TLR8 agonist conjugate, HER2-DEC205G1-TLR8 agonist conjugate, or HER2-CLEC9AG1-TLR8 agonist conjugate is added to a well coated with HER2 antigen comprising mDCs; or a HER2-CD40G1-STING agonist conjugate, HER2-DEC205G1-STING agonist conjugate, or HER2-CLEC9AG1-STING agonist conjugate is added to a well coated with HER2 antigen comprising mDCs. As controls, monospecific tumor antigen antibody is added to wells coated with tumor antigen or the bispecific recombinant antibody constructs described in this example are added to wells coated with an irrelevant tumor antigen. After 48 hours incubation in growth media, the cells are collected and washed by centrifugations and are then stained for 30 minutes on ice using manufacturer's recommended concentrations of commercially available anti-CD54, anti-CD80, anti-CD83, anti-CD86 and anti-MHC class II (anti-HLA-DR) monoclonal antibodies conjugated to laser sensitive fluors and are stained for viability. A separate aliquot for each treatment is stained with IgG matched isotype control antibody conjugate for anti-CD54, anti-CD80, anti-CD83, anti-CD86 and anti-MHC class II (anti-HLA-DR). After washing to remove unbound antibody-fluor molecules the stained cells are subjected to FACS analysis using a Celesta flow cytometer (BD Biosciences) with gating on live cells. The output was analyzed by FlowJo v10.2 software (FlowJo LLC) and curve fit with Prism 7.01 software (GraphPad Software, Inc.). When the bispecific tumor targeting conjugates are bound to tumor antigen, the DC expression of CD54, CD80, CD86, MHC Class II (HLA-DR), and CD83 is increased in equal or greater amounts than the DC expression of CD54, CD80, CD86, MHC Class II (HLA-DR), and CD83 in wells comprising the bispecific tumor targeting conjugates and an irrelevant tumor antigen or wells comprising monospecific tumor antigen antibody and tumor antigen.

### Example 26

### Pattern Recognition Receptors are Stimulated by Bispecific Recombinant Antibody when the Bispecific Recombinant Antibody Conjugate is Bound to Tumor Antigen

This example shows that pattern recognition receptors (PRRs) are stimulated by bispecific recombinant antibody conjugates when the bispecific recombinant antibody conjugates are bound to tumor antigen. The bispecific recombinant antibody conjugates with STING agonists attached are HER2-CD40G1-STING agonist conjugate, EGFRvIII-CD40G1-STING agonist conjugate, or CEA-CD40G1-STING agonist conjugate, which are tested for their ability to activate the PAMP signal using tumor cell lines bearing HER2, EGFRvIII, or CEA. Each conjugate without the CD40G1 antigen binding domain (i.e., HER2-STING agonist conjugate, EGFRvIII-STING agonist conjugate, and CEA-STING agonist conjugate) is used as a control. Titrations of the bispecific recombinant antibody conjugates with STING agonists or control conjugates are added to 10⁵ tumor cells in wells of a 96 well plate followed by 24 hour incubation (QPCR), 0-180 minutes (Western Blot) or 48 hour (FACS) incubations. Three assay formats were used to demonstrate tumor cell response to the PAMP/DAMP immune stimulators. In the first mRNA is isolated from the tumor cells after bispecific recombinant antibody conjugates conjugated with an immune-stimulatory compound and control treatment and QPCR is performed to quantitate type 1 and type 2 interferon mRNAs. In the second cell lysates are made, the proteins analyzed by Western blot analysis for relative levels of phosphor-TBK1 and phosphor-IRF3. In the third the relative levels of MHC Class I on the treated tumor cells is measured by FACS. For QPCR analysis mRNA is isolated, reverse transcribed and subjected QPCR analysis with fluor labeled primers and probes for type 1 and type 2 interferon using commercially available reagents (GoTaq Probe, Promega) and QPCR thermocycler (Roche LightCycler 480) and comparing to QPCR for “housekeeping gene” pair ACTB-GAPDH. For phospho-protein analysis proteins are extracted with triton cell lysis buffer containing proteinase (Thermo Fisher) and phosphatase (Sigma) inhibitors, 30 mg of protein is separated by SDS-PAGE, transferred to Immobilon FL membranes (Millipore) and the membrane is incubated with commercially available for phosphorylated TBK1, phosphorylated IRF3, STING, and GAPDH. After washing the bound antibodies are detected with anti-rabbit IRDye 680D and an Imaging system. FACS analysis is performed after dissociating the cells from the well using Accutase, incubating on ice for 30 minutes with fluor-conjugated mouse anti-human HLA-ABC (BD Biosciences). The results of the analysis show that the bispecific recombinant antibody conjugates retain the ability to activate immune stimulating activities in tumor cells specifically when bound to their tumor antigen.

### Example 27

### Immune Activation Capabilities of Immune Suppressive Cells are Increased when Bispecific Recombinant Antibody Constructs or Bispecific Recombinant Antibody Conjugates are Bound to their Specific Antigen

This example shows that the immune activation capabilities of immune suppressive cells are increased when bispecific recombinant antibody constructs are bound to their specific tumor antigen or when bispecific recombinant antibody conjugates are bound to their specific tumor antigen.

Activating immune-stimulatory cytokines and activating cell surface immune-stimulatory molecules important for T cell antigen responses from immunosuppressive Myeloid Derived Suppressive Cells (MDSC) are increased when bispecific recombinant antibody constructs are bound to their specific tumor antigen by their first antigen binding domain or when bispecific recombinant antibody conjugates conjugated with a TLR8 agonist or STING agonist are bound to their specific tumor antigen by their first antigen binding domain. Wells are coated with tumor antigen as described in EXAMPLE 24. Human peripheral blood mononuclear cells (PBMCs) are isolated as in EXAMPLE 38. MDSC are derived from PBMCs by isolation of CD14⁺ monocytes. The monocytes are cultured in T-25 flasks at 5×10⁵ cells/ml in complete medium (RPMI+10% FBS+P/S+L-Glut+Na-Pyr) for 7 d, supplemented with IL-6 (10 ng/ml; Peprotech) and GM-CSF (10 ng/ml; Peprotech). Medium and cytokines are refreshed every 2-3 d. On day 7 of culture, MDSC are lifted and disassociated using HyQTase (GE Life Sciences) and washed twice with complete media. To determine conditional immune stimulation, MDSCs are plated at 5×10⁴ cells/well into 96 well plates where the individual wells contained cells with surface expression of either HER2, CEA, or MSLN tumor antigens. The following bispecific recombinant antibody constructs are added at varying concentrations to the wells coated with the relevant tumor antigen: HER2-CD40G1, CEA-CD40G1, MSLN-CD40G1, HER2-DEC205G1, CEA-DEC205G1, MSLN-DEC205G1, HER2-CLEC9AG1, CEA-CLEC9AG1, or MSLN-CLEC9AG1. Additionally, each of these bispecific recombinant antibody conjugates conjugated with a TLR8 agonist or STING agonist are added to aliquots of MDSC and tumor antigen for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots of MDSCs and tumor antigens as controls. As an additional control, bispecific recombinant antibody conjugates were added wells containing MDSCs and irrelevant tumor antigen. After 24 hours of co-culture supernatants are removed and assayed for the cytokines IL-12p70, IL-10 and TNFα as described in EXAMPLE 22. In addition, the MDSCs are removed from the wells by dissociation with HyQtase (GE Life Sciences) then subjected to FACS analysis as described in EXAMPLE 25 using commercially available reagents for CD33, CD11b, CD80, CD86, MHC Class II, and viable cells. The level of CD80, CD86, and MHC Class II is assessed for viable MDSC defined as viable dye negative CD33⁺ CD11b cells. As is shown by the results, IL-12p70 and TNFα production, and CD80, CD86, and MHC Class II density on MDSCs is increased when bispecific recombinant antibody constructs are bound to their specific tumor antigen. Similar results are shown when bispecific recombinant antibody conjugates attached to a TLR8 agonist or a STING agonist are bound to their specific tumor antigen.

### Example 28

### Immune Suppressive Activity of Immune Suppressive Cells is Decreased when Bispecific Recombinant Antibody Conjugates are Bound to their Specific Tumor Antigen

This example shows that the immune suppressive activity of immune suppressive cells is decreased when bispecific recombinant antibody constructs are bound to their specific tumor antigen or when bispecific recombinant antibody conjugates are bound to their specific tumor antigen.

Myeloid Derived Suppressive Cell (MDSC) suppression of T cell activation is reduced when bispecific recombinant antibody constructs are bound to their specific tumor antigen by their second binding or when bispecific recombinant antibody conjugates with a TLR8 agonist or a STING agonist are bound to their specific tumor antigen by their first binding domain. Human PBMCs are isolated as in EXAMPLE 38. MDSC are generated as in EXAMPLE 27. MDSCs are harvested from the flasks using HyQTase (GE Life Sciences) and washed twice with complete media. Dilutions of the MDSC suspended in growth media beginning at 5×10⁴ are added to tumor antigen coated wells of a round bottom 96 well plates followed by incubation at 37° C. 5% CO₂ for 24 hours. The tumor antigen coating is performed with 1 ug/ml of soluble tumor cell antigens as described in EXAMPLE 25. The following bispecific recombinant antibody conjugates are added to aliquots of the MDSC with relevant tumor antigen for testing: HER2-CD40G1, CEA-CD40G1, MSLN-CD40G1, HER2-DEC205G1, CEA-DEC205G1, MSLN-DEC205G1, HER2-CLEC9AG1, CEA-CLEC9AG1, and MSLN-CLEC9AG1. Additionally, each of these bispecific recombinant antibody conjugates with a TLR8 agonist or STING agonist are added to aliquots of MDSCs and relevant tumor antigen for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots of MDSC and relevant tumor antigen as controls. After incubation of each well, a T cell suppression assay is performed as follows. Donor-matched T cells are isolated by negative selection (STEMCELL) and labeled with CellTrace Violet (ThermoFisher) according to the manufacturer's directions. 5×10⁴ labeled T cells are added to each MDSC-containing well to generate various MDSC to T cell ratios (i.e., 1:1, 1:2, 1:4, 1:16, 1:32). T cell-alone wells are included as a control for maximal proliferation. Cultures are stimulated by adding 5×10⁴ Dynabeads Human T-Activator CD3/CD28 (ThermoFisher) to each well. Cultures are then incubated at 37° C. for 4 days. On day 4, T cell proliferation is assessed by measuring proliferation dye dilution using FACS. As is shown by the results, a loss of suppressive function, as evidenced by increased numbers of T cells with lower MFIs for CellTrace Violet due to increased cell divisions, is found for T cells from wells incubated with bispecific recombinant antibody constructs and their tumor antigen. Similar results are shown incubation with bispecific recombinant antibody conjugates with a TLR8 agonist or STING agonist and their tumor antigen.

### Example 29

### Accumulation of Bispecific Tumor Targeting is Increased in Tumors in Comparison with Non-Tumor Targeting Anti-CD40 Conjugates

This example shows that the level of bispecific tumor targeting conjugates found in tumors is increased in comparison with the level of non-tumor targeting anti-CD40 conjugates found in tumors. Additionally, this example shows that the tumor/spleen ratio of bispecific tumor targeting conjugates is increased in comparison with the tumor/spleen ratio of non-tumor targeting anti-CD40 conjugates. Increased levels of the bispecific tumor targeting conjugate in tumors or in associated secondary lymphoid organs, where tumor antigens and tumor derived immunosuppression can be maximal, can help increase tumor innate and adaptive immune responses while limiting toxicities found with systemic immune activation.

Tumor accumulation of a HER2-CD40 conjugate with motolimod is compared to a control CEA-CD40 conjugate with motolimod and a control anti-CD40 conjugate with motolimod in an orthotopic HER2+ C57B16 syngeneic model using transgenic mice humanized for CD40. Transfectants of the b6 breast tumor cell line E0711 tumor line expressing human HER2 are generated by standard transfection protocols followed by drug selection and soft agar cloning. The human tumor antigen expressing line is implanted orthotopically in mammary fat pads in huCD40 tg mice at a dose of 1×10⁶ cells/mouse. The tumor volume is measured by calipers. Mice with tumors of approximately 200 mm² are intravenously injected with approximately 5-10 μCi of HER2-C40G1 conjugate with motolimod, CEA-CD40G1 conjugate with motolimod, or anti-CD40 antibody conjugate with motolimod, in which each are radiolabeled with 125I by Bolton Hunter technology to specific activity of approximately 1 uCi/μg. Mice are euthanized at 0 hours, at 24 hours or at 48 hours after injection. Blood, spleen and tumors are collected and the relative levels of the radiolabeled compositions or antibodies are quantitated by a gamma counter. A higher percentage of input tracer in the tumors and a greater tumor/spleen ratio is found in mice injected with HER2-C40G1 conjugate with motolimod as compared with the CEA-CD40G1 conjugate with motolimod or the anti-CD40 antibody conjugate with motolimod.

### Example 30

### The Anti-Tumor Response is Enhanced by Bispecific Tumor Targeting Conjugates

This example shows the anti-tumor immune response is enhanced by bispecific tumor targeting antibody constructs or by bispecific tumor targeting conjugates.

A human immune cell mediated anti-tumor response in tumor xenograft models using immunocompromised mice is enhanced by bispecific tumor targeting antibody constructs or by bispecific tumor targeting conjugates with a TLR8 agonist or STING agonist. In immunocompromised mice, 1×10⁷ cells/animal human tumor cells from lines bearing either HER2, CEA or EGFRvIII are co-injected subcutaneously into the flanks of mice from strain NSG (NOD.Cg-Prkdc^{scid}IL-2rg^{tm1Wj1}) along with donor matched human T cells (1×10⁶/animal) and mDCs (5×10⁵/animal). The incorporation of human mDCs in this model allows for the assessment of in vivo immune-mediated activity of bispecific tumor targeting antibody constructs or bispecific tumor targeting conjugates with a TLR8 agonist or STING agonist. Bispecific tumor targeting antibody constructs or by bispecific tumor targeting conjugates with a TLR8 agonist or STING agonist bearing one of the following first antigen specificities (HER2, CEA, EGFRvIII) and one of the following second antigen specificities (CD40, DEC205) is injected intraperitoneally immediately prior to tumor injection. As a control, bispecific tumor targeting antibody constructs or by bispecific tumor targeting conjugates with a TLR8 agonist or STING agonist in which the second antigen binding domain is not directed to antigen on the injected tumor is injected intraperitoneally immediately prior to tumor injection. Tumor growth is measured using calipers twice per week, beginning seven days post tumor cell transfer and ending at study termination, approximately 3 weeks after tumor cell inoculation. As is shown by the results, tumor growth is reduced only when the injected tumor cells express the first antigen specificity of the bispecific tumor targeting antibody constructs or of the bispecific tumor targeting conjugates with a TLR8 agonist or STING agonist.

### Example 31

### Treatment of HER2 Expressing Cancer by Administering a Bispecific Tumor Targeting Conjugate

This example describes treatment of a HER2 expressing cancer with a bispecific tumor targeting conjugate. A human patient is diagnosed with a cancer that expresses the tumor-associated antigen p185HER2, such as breast cancer or gastric cancer. A bispecific tumor targeting conjugate comprising a HER2-CD40 conjugate, which specifically binds the tumor-associated antigen p185HER2, conjugated with a TLR8 agonist or STING agonist is administered to the patient. An anti-tumor response is induced against tumor cells expressing p185HER2, such as a T cell-mediated immune response and an innate immune response, leading to control and eradication of tumor cells.

### Example 32

### Treatment of DLL3 Expressing Cancer by Administering a Bispecific Tumor Targeting Conjugate

This example describes treatment of a DLL3 expressing cancer with a bispecific tumor targeting conjugate. A human patient is diagnosed with a cancer that expresses the tumor-associated antigen DLL3, such as small cell lung cancer. A bispecific tumor targeting conjugate comprising DLL3-DEC-205 conjugate with a TLR8 agonist or STING agonist is administered to the patient. An anti-tumor response is induced against tumor cells expressing DLL3, such as a T cell-mediated immune response and an innate immune response, leading to control and eradication of tumor cells.

### Example 33

### Stability of Bispecific Tumor Targeting Conjugate Conjugated with an Immune-Stimulatory Compound Conjugate in IgG Depleted Human Serum

This example shows the stability of a bispecific tumor targeting antibody construct and bispecific tumor targeting conjugate in IgG depleted human serum. Stability of a bispecific tumor targeting antibody construct and bispecific tumor targeting conjugate in human serum (IgG depleted) is measured over 96 hours at 37° C. using either a direct HIC-UV analysis approach (Method A) or an affinity capture approach (Method B). Bispecific tumor targeting antibody constructs or bispecific tumor targeting conjugates are spiked in IgG-depleted human serum (BBI solutions # SF142-2) in sterile tubes (75% final serum concentration) and samples are split into 4 aliquots of equal size then transferred to a 37° C. incubator. One of the aliquots of each sample is taken from the incubator at each time-point (T=0h, 24h, 48h, 96h) and the stability of the bispecific tumor targeting antibody construct or the bispecific tumor targeting conjugate, or the average immune-stimulatory compound-bispecific tumor targeting conjugate ratios (DAR), is recorded.

### Method A: Direct HIC-UV Analysis

At 0, 24, 48, and 96 hours after the beginning of incubation, bispecific tumor targeting antibody construct or bispecific tumor targeting conjugate spiked in IgG depleted human serum is analyzed by analytical hydrophobic interaction chromatography (HIC) using a TOSOH TSKgel Butyl-NPR 4.6 mm×35 mm HIC column (TOSOH Bioscience, #14947) connected to a Dionex Ultimate 3000RS HPLC system (ThermoFisher Scientific, Hemel Hemstead, UK). The stability of the bispecific tumor targeting conjugate is assessed and is found to remain stable at each time point. The average DAR is found to remain stable at each timepoint.

### Method B: Affinity Capture, De-Glycosylation and RP-ESI-MS Analysis

A bispecific tumor targeting antibody construct or bispecific tumor targeting conjugate is immunocaptured from the IgG depleted human serum using an anti-Human IgG (Fc specific) biotin antibody immobilized on streptavidin beads at 0, 24, 48 and 96 hours after the beginning of incubation. After elution from the beads, the samples are de-glycosylated using agarose-immobilized EndoS (Genovis Inc, USA). The de-glycosylated bispecific tumor targeting antibody construct or bispecific tumor targeting conjugate is analyzed by reverse phase chromatography hyphenated to electrospray ionization mass spectrometry (RP-ESI-MS) using an Acquity nano UPLC in line with a Xevo G2S Q-TOF (Waters, Elstree, UK). The separation is performed using an Acquity UPLC online coupled to an ESI-MS mass spectrometer. Mass spectrometric analysis is performed in positive ion mode, scanning from 1000 to 4000 m/z in high mass operating mode. The ion envelope produced by each sample is deconvoluted using the MaxEnt1 algorithm provided within the MassLynx software (Waters, Elstree, UK). The stability of the bispecific tumor targeting conjugate is assessed and is found to remain stable at each time point. The average DAR is found to remain stable at each timepoint.

### Example 34

### Bispecific Recombinant Antibody Constructs Showed Reduction in Binding to the Cellularly Expressed Antigen Presenting Cell Target CD40 Compared to Parental Anti-CD40 Monoclonal Antibody

This example shows that a bispecific recombinant antibody construct had reduced binding to the antigen presenting cell (APC) target CD40 when binding was measured on whole cells expressing native protein compared to parental anti-CD40 monoclonal antibody.

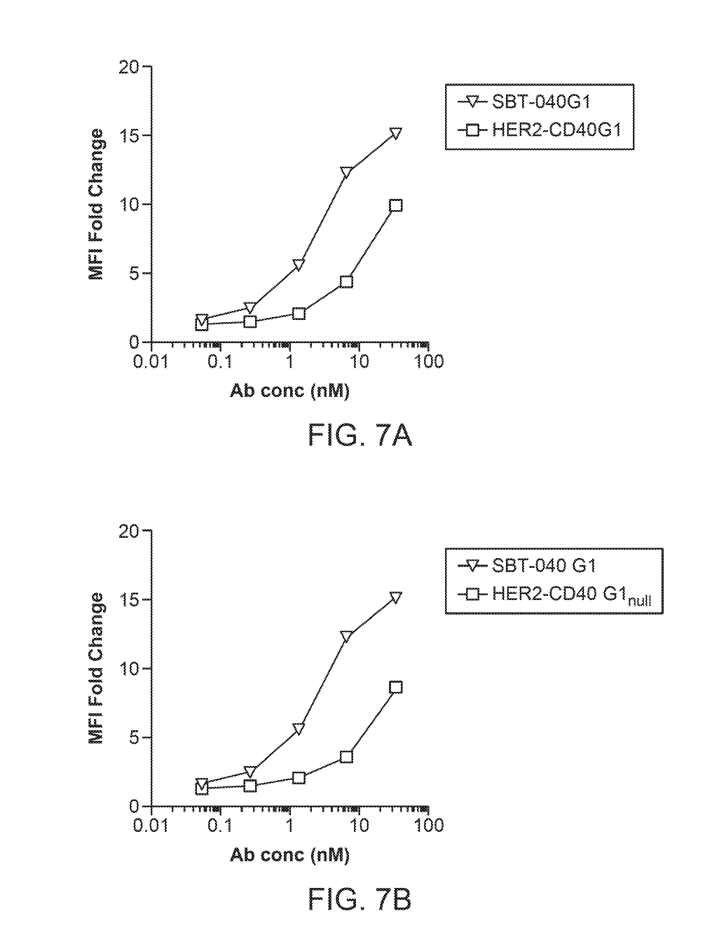

Bispecific anti-HER2-anti-CD40 IgG1 antibody construct (HER2-CD40G1) (prepared as described in Example 18) binds to CD40 expressed on dendritic cells less efficiently than the parental anti-CD40 monoclonal antibody (SBT-040G1). Monocytes were isolated from total human peripheral blood mononuclear cells (PBMCs) by magnetic bead-based negative selection. To generate monocyte-derived dendritic cells (moDCs), monocytes were cultured for 7 days in the presence of GM-CSF and IL-4. moDCs were stained with equivalent molar concentrations of either SBT-040G1 antibody or HER2-CD40G1 antibody construct followed by incubation with a PE-labeled goat anti-human IgG polyclonal detection antibody. Assessment of antibody binding by flow cytometry indicated a binding EC50 for SBT-040G1 antibody of 2.8 nM compared to a binding EC50 of 23.2 nM for HER2-CD40G1 antibody construct, and FIG. 7A also shows that HER2-CD40G1 antibody construct binds to CD40 expressed on dendritic cells less efficiently than SBT-040G1. Therefore, HER2-CD40G1 antibody construct binding to surface CD40 on moDCs was decreased compared to SBT-040G1 antibody.

### Example 35

### Generation of a Bispecific Anti-HER2×Anti-CD40 Fcnull Recombinant Antibody

This example shows a method for generating a bispecific recombinant antibody with a human Fcγ receptor (FcγR) binding null Fc domain. The anti-CD40 single chain Fv (scFv) in the VH-VL orientation was fused to the C-terminus of the anti-HER2 heavy chain (VH—CH1-CH2-CH3) (pertuzumab) containing the Fc null mutations to generate the anti-HER2 VH—CH1-CH2-CH3-anti-CD40 scFv heavy chain. The human IgG₁ null Fc domain was comprised the following mutations: L234A, L235A, G237A, and K322A.

This heavy chain was co-expressed with the light chain of anti-HER2 (VL-Ck) (pertuzumab) in the transient EpiCHO system in 180 mL of CHO media. The EpiCHO cells were transfected with heavy and light bispecific constructs using ExpiFectamine CHO reagent (Thermo Fisher) in 180 mL of CHO cell culture at a cell density of 6 million cells/mL. The supernatant was harvested 7 days after the transfection and the bispecific antibody was purified over HiScreen MabSelect SuRe Protein A column on a GE AKTA Pure machine. Protein was dialyzed against PBS and analyzed on Size Exclusion Chromatography which showed the % purity to be around 85%. 32 mg of protein was recovered giving a yield of 178 ug/mL expression from the harvested supernatant.

### Example 36

### Target Binding Affinity of a HER2×Anti-CD40 Fcnull Recombinant Bispecific Antibody

This example shows that the anti-HER2×anti-CD40 Fcₙᵤₗₗ recombinant bispecific antibody bound to monomeric human HER2 and the monomeric extracellular domain of human CD40 (CD40 ECD) with similar affinity as its parent anti-HER2 and anti-CD40 antibodies.

The anti-HER2×anti-CD40 Fcₙᵤₗₗ antibody was prepared as described in Example 35. Analysis of human HER2 and CD40 ECD interactions were performed using Octet Red 96 instrument (ForteBio). The Octet systems use propriety BLI to analyze biomolecular interaction. Anti-HER2 parental monoclonal antibody, anti-CD40 parental monoclonal antibody, and recombinant bispecific antibodies were immobilized on anti-human Fc biosensors and incubated with varying concentration of monomeric human HER2 and human CD40 ECD ranging from 1.2 nM to 300 nM in PBS/0.1% BSA/0.02% Tween 20. The experiments were performed using 5 steps: (1) baseline acquisition (60 s); (2) parental antibodies and bispecific antibodies loading onto anti-human Fc biosensor (120 s); (3) second baseline acquisition (60 s); (4) association of interacting monomeric HER2 and CD40 ECD proteins for kₒₙ measurement (120 s); and (5) dissociation of interacting monomeric HER2 and CD40 ECD for k_{off} measurement (300 s). The interacting monomeric HER2 and CD40 ECDs were used at 6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. A summary of this data is shown in TABLE 26. The binding of the bispecific recombinant antibody, anti-HER2×anti-CD40 Fcₙᵤₗₗ, to HER2 and CD40 ECD were similar to the binding of the parental anti-HER2 monoclonal antibody and anti-CD40 monoclonal antibody.

### Example 37

### Fc Receptor Binding Affinity of Anti-HER2×Anti-CD40 Fcnull Recombinant Bispecific Antibody

This example shows that the recombinant bispecific antibody anti-HER2×anti-CD40 Fcₙᵤₗₗ (HER2-CD40 Fcₙᵤₗₗ) containing the null mutations was not bound to Fcγ receptors as compared to a recombinant bispecific antibody with an intact Fc comprising domain.

The anti-HER2×anti-CD40 Fcₙᵤₗₗ antibody was prepared as described in Example 35. The anti-HER2×CD40 IgG antibody was prepared as described in Example 18. Human FcγR interaction analysis was performed using an Octet Red 96 instrument. For human FcγRI and FcγRIIA interactions, the HER2-CD40 Fcₙᵤₗₗ recombinant antibody or the bispecific recombinant antibody with an intact Fc comprising domain was immobilized on anti-human Fc biosensors and incubated with varying concentration of monomeric FcγR ranging from 1.2 nM to 300 nM in PBS/0.1% BSA/0.02% Tween 20. The experiments were performed using five steps: (1) baseline acquisition (60 s); (2) bispecific antibody loading onto anti-human Fc biosensor (120 s); (3) second baseline acquisition (60 s); (4) association of interacting FcγR for kₒₙ measurement (120 s); and (5) dissociation of interacting FcγR for k_{off} measurement (300 s). The interacting monomeric FcγRs were used at 4-6 concentrations of a 3-fold concentration series. The data were analyzed using Octet Data Analysis Software 9.0 (ForteBio)™ and fit to the 1:1 binding model. Equilibrium dissociation constants (K_{D}) were calculated by the ratio of kₒₙ to k_{off}.

For human FcγRIIIA F158 and FcγRIIIA V158 interaction studies, the FcγRs were immobilized on anti-His tag biosensors and incubated with varying concentrations of the HER2-CD40 Fcₙᵤₗₗ recombinant antibody or the recombinant bispecific antibody with an intact Fc comprising domain ranging from 37 nM to 1 μM. The experiment was performed using five steps: (1) Baseline acquisition (30 s); (2) human FcγRs loading to the anti-His tag biosensor (90 s); (3) second baseline acquisition (30 s); (4) association of interacting recombinant bispecific antibodies for kₒₙ measurement (30 s); and (5) dissociation of interaction recombinant bispecific antibodies for k_{off} measurement (30 s). The interacting HER2-CD40 Fcₙᵤₗₗ recombinant antibody or recombinant bispecific antibody with an intact Fc comprising domain was used at 4 concentrations of a 3-fold concentration series. The data were analyzed using Octet Data Analysis Software 9.0 (ForteBio)™ and fit to the avidity binding model. Equilibrium dissociation constants (K_{D}) were calculated by the ratio of kon to koff. The data are shown in TABLE 26 along with data for the control monospecific antibodies tested using the same protocol as just described. The recombinant bispecific antibody, HER2-CD40 Fcₙᵤₗₗ did not bind to the Fcγ receptors unlike the bispecific antibody, HER2-CD40₁ Fc IgG1 where there was an intact unmutated Fc region.

### Example 38

### Lower Immunostimulatory Cytokine is Elicited by Unbound IgG1 Fcnull Recombinant Bispecific Antibodies Compared with Immunostimulatory Cytokine Elicited by Unbound IgG1 Fc Recombinant Bispecific Antibodies

This example shows that lower immunostimulatory cytokine is produced by human dendritic cells (mDCs) in the presence of unbound IgG1 Fcₙᵤₗₗ recombinant bispecific antibodies as compared with immunostimulatory cytokine produced by mDCs in the presence of unbound IgG1 Fc recombinant bispecific antibodies. This example also shows that lower immunostimulatory cytokine is produced by human dendritic cells (mDCs) in the presence of unbound IgG1 Fcₙᵤₗₗ recombinant bispecific antibodies conjugated with an immunostimulatory molecule as compared with immunostimulatory cytokine produced by mDCs in the presence of unbound IgG1 Fc recombinant bispecific antibodies conjugated with an immunostimulatory molecule.

Human peripheral blood mononuclear cells (PBMCs) are isolated from normal donors by a standard Ficoll-Hypaque protocol. Dendritic cells (mDCs) are derived from CD14⁺ monocytes isolated from PBMCs by negative selection using a commercially available kit. The monocytes are cultured in RPMI containing 10% fetal calf serum for seven days in complete medium supplemented with 25 ng/mL IL-4 and 10 ng/mL GM-CSF. The media is replaced with fresh media plus cytokines on day three. On day six the following bispecific recombinant antibodies are added to aliquots of the mDCs for testing: HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, MSLN-CLEC9A G1ₙᵤₗₗ, and their respective bispecific recombinant antibody with a G1 Fc domain instead of an G1ₙᵤₗₗ Fc domain. Additionally, each of these bispecific recombinant antibodies conjugated with a TLR8 agonist or STING agonist are added to aliquots of mDCs for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots of mDCs as controls. After a 24 hour incubation, supernatants are collected and the cytokines IL-6, TNFα, IL-12p70, and IL-10 produced by the dendritic cells are quantitated by electrochemiluminesence signal by multiplex ELISA using commercially available reagents and plate reader from Meso Scale Discovery. Lower amounts of immunostimulatory cytokine is shown to be produced by human mDCs when bispecific IgG1 Fcₙᵤₗₗ recombinant antibodies are added as compared with immunostimulatory cytokine produced by human dendritic cells (mDCs) bispecific IgG1 Fc recombinant antibodies. Additionally, lower amounts of immunostimulatory cytokine is shown to be produced by human mDCs when bispecific IgG1 Fcₙᵤₗₗ recombinant antibodies conjugated to TLR8 agonists or STING agonists are added as compared with immunostimulatory cytokine produced by human dendritic cells (mDCs) bispecific IgG1 Fc recombinant antibodies conjugated to TLR8 agonists or STING agonists or monospecific antibodies conjugated to TLR8 agonists or STING agonists.

### Example 39

### Dendritic Cell Immunostimulatory Cytokine Release is Enhanced when Bispecific Recombinant Antibodies Containing G1null Fc Domains are Bound to their Specific Tumor Antigen

This example shows that immunostimulatory cytokine produced by human dendritic cells (mDCs) is enhanced when IgG1 Fcₙᵤₗₗ recombinant bispecific antibodies are bound to their specific tumor antigen or when IgG1 Fcₙᵤₗₗ recombinant bispecific antibodies conjugated with an immune stimulatory molecule are bound to their specific tumor antigen.

mDC cytokine production is enhanced when bispecific recombinant antibodies lacking FcγR binding (i.e., Fcₙᵤₗₗ) are bound to their specific tumor antigen by their first antigen binding domain or when recombinant bispecific antibodies lacking FcγR binding (i.e., Fcₙᵤₗₗ) conjugated with a TLR8 agonist or STING agonist are bound to their specific tumor antigen by their first antigen binding domain. Human PBMCs are isolated as in EXAMPLE 38. mDCs are derived from CD14⁺ monocytes isolated from the peripheral blood mononuclear cells (PBMCs) by negative selection using a commercially available kit. The monocytes are cultured in RPMI containing 10% fetal calf serum for seven days in complete medium supplemented with 25 ng/mL IL-4 and 10 ng/mL GM-CSF. The media is replaced with fresh media plus cytokines on day three. On day six, 5×10⁴ dendritic cells are added in the growth media to separate wells of a 96 well microtiter plate that is coated with varying concentrations of commercially available recombinant soluble HER2 ectodomain, a soluble CEA ectodomain or a soluble MSLN ectodomain. The coating is performed by incubation of the added soluble tumor antigen at 1 ug/ml for 1 hour at room temperature, followed by 3 washes to remove any unbound protein. The following bispecific recombinant antibodies containing G1ₙᵤₗₗ Fc domains added at varying concentrations to the wells coated with the relevant tumor antigen: HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, or MSLN-CLEC9A G1ₙᵤₗₗ, or any of these bispecific recombinant antibodies conjugated with a TLR8 agonist or STING agonist. The corresponding recombinant bispecific antibodies with G1 Fc domains or monospecific antibodies with G1 Fc domains, or these bispecific or monospecific antibodies conjugated with a TLR8 agonist or STING agonist are added at varying concentrations to wells coated with the relevant tumor antigen as controls. As an additional control, G1ₙᵤₗₗ Fc domain recombinant bispecific antibodies or G1ₙᵤₗₗ Fc domain recombinant bispecific antibodies conjugated with a TLR8 agonist or STING agonist are added to wells containing mDCs and irrelevant tumor antigen. After 24 hours incubation in mDC growth media at 37° C. with 5% CO₂ atmosphere supernatants are collected and the cytokines IL-6, TNFα, IL-12p70, and IL-10 produced by the mDCs are quantitated by electrochemiluminesence signal by multiplex ELISA using commercially available reagents and plate reader from Meso Scale Discovery. Immunostimulatory cytokine produced by mDCs is shown by the results to be enhanced when bispecific IgG1 Fcₙᵤₗₗ recombinant antibodies are bound to their specific tumor antigen. Similar results are shown when bispecific IgG1 Fcₙᵤₗₗ recombinant antibodies conjugated with a TLR8 agonist or STING agonist are bound to their specific tumor antigen.

### Example 40

### Cell Surface Immunomodulatory Molecule Expression is Increased when IgG1 Fcnull Recombinant Bispecific Antibodies are Bound to their Specific Tumor Antibodies

This example shows that the expression of cell surface immunomodulatory molecules on human myeloid dendritic cells (mDCs) is increased when bispecific IgG1 Fcₙᵤₗₗ recombinant antibodies are bound to their specific tumor antigen or when bispecific IgG1 Fcₙᵤₗₗ antibodies conjugated with an immune stimulatory molecule are bound to their specific tumor antigen.

mDC cell surface CD54, CD86, CD80, MHC Class II (HLA-DR) and CD83 expression are increased, in a dose dependent manner, when the first antigen binding domain of a bispecific IgG1 Fcₙᵤₗₗ recombinant antibody is bound to its specific tumor antigen or when the first antigen binding domain of a bispecific IgG1 Fcₙᵤₗₗ recombinant antibody conjugated with a TLR8 agonist or STING agonist is bound to its specific tumor antigen. Human peripheral blood mononuclear cells (PBMCs) are isolated as in EXAMPLE 38. mDCs are derived from PBMCs by isolation of CD14⁺ monocytes followed by culture in RPMI containing 10% fetal calf serum for seven days in complete medium supplemented with 10 ng/mL IL-4 and 100 ng/mL GM-CSF. After three days of culture the media is removed and replaced with fresh media including cytokine supplement. The following bispecific recombinant antibodies are added at varying concentrations to the wells coated with the relevant tumor antigen: HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, or MSLN-CLEC9A G1ₙᵤₗₗ, HER2-CD40 G1, CEA-CD40 G1, MSLN-CD40 G1, HER2-DEC205 G1, CEA-DEC205 G1, MSLN-DEC205 G1, HER2-CLEC9A G1, CEA-CLEC9A G1, or MSLN-CLEC9A G1. Additionally, each of these bispecific recombinant antibodies further comprising either an attached TLR8 agonist or STING agonist are added to aliquots of mDCs for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots with varying concentrations of mDCs as controls. As an additional control, bispecific G1ₙᵤₗₗ Fc domain recombinant antibodies were added to wells containing mDCs and irrelevant tumor antigen. After 48 hours of incubation in growth media the cells are collected and washed by centrifugations then stained for 30 minutes on ice using manufacturer's recommended concentrations of commercially available anti-CD83, anti-CD86 and anti-MHC class II monoclonal antibodies conjugated to laser sensitive fluors as well as stained for viability. A separate aliquot for each treatment is stained with IgG matched isotype control antibody conjugate for anti-CD54, anti-CD80, anti-CD86, anti-CD83 and anti-MHC Class II (HLA-DR). After washing to remove unbound antibody-fluor molecules the stained cells are subjected to FACS analysis using a Celesta flow cytometer (BD Biosciences) with gating on live cells. The output is analyzed by FlowJo v10.2 software (FlowJo LLC) and curve fit with Prism 7.01 software (GraphPad Software, Inc.). As shown by the results, mDC cell surface CD54, CD80, CD86, MHC Class II (HLA-DR), and CD83 expression is increased, in a dose dependent manner, by the addition of bispecific IgG1 Fcₙᵤₗₗ recombinant antibody to wells containing its specific tumor antigen. Similar results are shown by the addition of bispecific IgG1 Fcₙᵤₗₗ recombinant antibody conjugated with a TLR8 agonist or STING agonist.

### Example 41

### Immune Activation Capabilities of Immune Suppressive Cells are Increased when Bispecific IgG1 Fcnull Recombinant Antibodies are Bound to their Specific Tumor Antigen

This example shows that the immune activation capabilities of immune suppressive cells are increased when bispecific IgG1 Fcₙᵤₗₗ recombinant antibodies are bound to their specific tumor antigen or when bispecific IgG1 Fcₙᵤₗₗ recombinant antibodies conjugated with an immune stimulatory molecule are bound to their specific tumor antigen.

Activating immunostimulatory cytokines and activating cell surface immunostimulatory molecules important for T cell antigen responses from immunosuppressive Myeloid Derived Suppressive Cells (MDSC) are increased when bispecific IgG1 Fcₙᵤₗₗ recombinant antibodies are bound to their specific tumor antigen by their first antigen binding domain or when bispecific IgG1 Fcₙᵤₗₗ recombinant antibodies conjugated with a TLR8 agonist or STING agonist are bound to their specific tumor antigen by their first antigen binding domain. Wells are coated with tumor antigen as described in EXAMPLE 25. Human peripheral blood mononuclear cells (PBMCs) are isolated as in EXAMPLE 38. MDSC are derived from PBMCs by isolation of CD14⁺ monocytes. The monocytes are cultured in T-25 flasks at 5×10⁵ cells/ml in complete medium (RPMI+10% FBS+P/S+L-Glut+Na-Pyr) for 7 d, supplemented with IL-6 (10 ng/ml; Peprotech) and GM-CSF (10 ng/ml; Peprotech). Medium and cytokines are refreshed every 2-3 d. On day 7 of culture, MDSC are lifted and disassociated using HyQTase (GE Life Sciences) and are washed twice with complete media. To determine conditional immune stimulation, MDSCs are plated at 5×10⁴ cells/well into 96 well plates where the individual wells contained cells with surface expression of either HER2, CEA, or MSLN tumor antigens. The following bispecific recombinant antibodies are added at varying concentrations to the wells coated with the relevant tumor antigen: HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, or MSLN-CLEC9A G1ₙᵤₗₗ, HER2-CD40 G1, CEA-CD40 G1, MSLN-CD40 G1, HER2-DEC205 G1, CEA-DEC205 G1, MSLN-DEC205 G1, HER2-CLEC9A G1, CEA-CLEC9A G, or MSLN-CLEC9A G1. Additionally, each of these bispecific recombinant antibodies conjugated with a TLR8 agonist or STING agonist are added to aliquots of MDSC and tumor antigen for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots of MDSCs and tumor antigens as controls. As an additional control, bispecific G1ᵤ₁₁ Fc domain recombinant antibodies are added wells containing MDSCs and irrelevant tumor antigen. After 24 hours of co-culture supernatants are removed and are assayed for the cytokines IL-12p70, IL-10, and TNFα as described in Example 5. In addition, the MDSCs are removed from the wells by dissociation with HyQtase (GE Life Sciences) and then are subjected to FACS analysis as described in Example 6 using commercially available reagents for CD33, CD11b, CD80, CD86, MHC Class II, and viable cells. The level of CD80, CD86, and MHC Class II is assessed for viable MDSC defined as viable dye negative CD33+CD11b′ cells. As is shown by the results, IL-12p70 and TNFα production, and CD80, CD86, and MHC Class II density on MDSCs is increased when bispecific IgG1 Fcₙᵤₗₗ recombinant antibodies are bound to their specific tumor antigen. Similar results are shown when bispecific IgG1 Fcₙᵤₗₗ recombinant antibodies attached to a TLR8 agonist or a STING agonist are bound to their specific tumor antigen

### Example 42

### Immune Suppressive Activity of Immune Suppressive Cells is Decreased when Bispecific IgG1 Fcnull Recombinant Antibodies are Bound to their Specific Tumor Antigen

This example shows that the immune suppressive activity of immune suppressive cells is decreased when bispecific IgG1 Fcₙᵤₗₗ recombinant antibodies are bound to their specific tumor antigen or when bispecific IgG1 Fcₙᵤₗₗ recombinant antibodies conjugated with an immune stimulatory molecule are bound to their specific tumor antigen.

Myeloid Derived Suppressive Cell (MDSC) suppression of T cell activation is reduced when bispecific IgG1 Fcₙᵤₗₗ recombinant antibodies are bound to their specific tumor antigen by their first antigen binding domain or when bispecific IgG1 Fcₙᵤₗₗ recombinant antibodies conjugated with a TLR8 agonist or a STING agonist are bound to their specific tumor antigen by their first antigen binding domain. Human PBMCs are isolated as in EXAMPLE 38. MDSC are generated as in EXAMPLE 27. MDSCs are harvested from the flasks using HyQTase (GE Life Sciences) and are washed twice with complete media. Dilutions of the MDSC suspended in growth media beginning at 5×10⁴ are added to tumor antigen coated wells of a round bottom 96 well plates followed by incubation at 37° C. 5% CO₂ for 24 hours. The tumor antigen coatings is performed with 1 ug/ml of soluble tumor cell antigens as described in EXAMPLE 25. The following bispecific recombinant antibodies are added to aliquots of the MDSC with relevant tumor antigen for testing: HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, MSLN-CLEC9A G1ₙᵤₗₗ, and their respective bispecific recombinant antibody with a G1 Fc domain instead of an G1ₙᵤₗₗ Fc domain. Additionally, each of these bispecific recombinant antibodies conjugated with a TLR8 agonist or STING agonist are added to aliquots of MDSCs and relevant tumor antigen for testing. Anti-CD40 agonist antibody conjugated with a TLR8 agonist or STING agonist, anti-DEC-205 antibody conjugated with a TLR8 agonist or STING agonist, anti-CLEC9A antibody conjugated with a TLR8 agonist or STING agonist, or HER2 antibody conjugated with a TLR8 agonist or STING agonist are added to aliquots of MDSC and relevant tumor antigen as controls. After incubation of each well, a T cell suppression assay is performed as follows. Donor-matched T cells are isolated by negative selection (STEMCELL) and labeled with CellTrace Violet (ThermoFisher) according to the manufacturer's directions. 5×10⁴ labeled T cells are added to each MDSC-containing well to generate various MDSC to T cell ratios (i.e., 1:1, 1:2, 1:4, 1:16, 1:32). T cell-alone wells are included as a control for maximal proliferation. Cultures are stimulated by adding 5×10⁴ Dynabeads Human T-Activator CD3/CD28 (ThermoFisher) to each well. Cultures are then incubated at 37° C. for 4 days. On day 4, T cell proliferation is assessed by measuring proliferation dye dilution using FACS. As is shown by the results, a loss of suppressive function, as evidenced by increased numbers of T cells with lower MFIs for CellTrace Violet due to increased cell divisions, is found for T cells from wells incubated with bispecific IgG1 Fcₙᵤₗₗ recombinant antibodies and their tumor antigen. Similar results are shown incubation with bispecific IgG1 Fcₙᵤₗₗ recombinant antibodies conjugated to a TLR8 agonist or STING agonist and their tumor antigen.

### Example 43

### Systemic Immune Activation In Vivo is Reduced by Bispecific IgG1 Fcnull Recombinant Antibodies

This example shows that systemic immune activation in vivo is reduced by bispecific IgG1 Fcₙᵤₗₗ recombinant antibodies or by bispecific IgG1 Fcₙᵤₗₗ recombinant antibodies conjugated with an immune stimulatory molecule.

Systemic activation of immune cells can limit tolerated doses in patients to levels that compromise therapeutic benefit. However, systemic activation of immune cells after administration of bispecific IgG1 Fcₙᵤₗₗ recombinant antibodies is reduced as compared to activation after administration of bispecific IgG1 Fc recombinant antibodies. Additionally, systemic activation of immune cells after administration of bispecific IgG1 Fcₙᵤₗₗ recombinant antibodies conjugated with a TLR8 agonist or STING agonist is reduced as compared to activation after administration of bispecific IgG1 Fc recombinant antibodies. Transgenic mice humanized for CD40 by replacement of mouse CD40 by the human CD40 are injected intravenously with 0.2 mg/kg, 2.0 mg/kg and 20 mg/kg of HER2-CD40 G1ₙᵤₗₗ, CEA-CD40 G1ₙᵤₗₗ, MSLN-CD40 G1ₙᵤₗₗ, HER2-DEC205 G1ₙᵤₗₗ, CEA-DEC205 G1ₙᵤₗₗ, MSLN-DEC205 G1ₙᵤₗₗ, HER2-CLEC9A G1ₙᵤₗₗ, CEA-CLEC9A G1ₙᵤₗₗ, MSLN-CLEC9A G1ₙ₁₁, or their respective bispecific recombinant antibody with a G1 Fc domain instead of an G1ₙᵤₗₗ domain. Additionally, 0.2 mg/kg, 2.0 mg/kg and 20 mg/kg of each of these bispecific antibodies conjugated with a TLR8 agonist or STING agonist are intravenously injected into transgenic mice. Immune activation is monitored by observation of general health followed by euthanization at 24 hours and ascertaining immune activation of B and myeloid cell types from the spleen by FACS staining for CD86, CD80, MHC Class II, and intracellular levels of TNFα and IL-12p40. Cytokines IL-6, TNFα, IL-12p70, and IL-10 levels in serum are also quantitated by electrochemiluminesence signal by multiplex ELISA using commercially available reagents and plate reader from Meso Scale Discovery. As shown by the results, systemic immune activation is reduced after administration of bispecific IgG1 Fcₙᵤₗₗ recombinant antibodies. Similar results are shown after administration of bispecific IgG1 Fcₙᵤₗₗ recombinant antibodies conjugated with a TLR8 agonist or a STING agonist.

### Example 44

### The Anti-Tumor Immune Response is Enhanced by Bispecific IgG1 Fcnull Recombinant Antibodies

This example shows the anti-tumor immune response is enhanced by bispecific IgG1 Fcₙᵤₗₗ recombinant antibodies or by bispecific IgG1 Fcₙᵤₗₗ antibodies conjugated with an immune stimulatory molecule.

A human immune cell mediated anti-tumor response in tumor xenograft models using immunocompromised mice is enhanced by bispecific IgG1 Fcₙᵤₗₗ recombinant antibodies or by bispecific IgG1 Fcₙᵤₗₗ recombinant antibodies conjugated with a TLR8 agonist or STING agonist. In immunocompromised mice, 1×10⁷ cells/animal human tumor cells from lines bearing either HER2, CEA or EGFRvIII are co-injected subcutaneously into the flanks of mice from strain NSG (NOD.Cg-Prkdc^{scid}IL-2rg^{tm1Wj1}/SzJ) along with donor matched human T cells (1×10⁶/animal) and mDCs (5×10⁵/animal). The incorporation of human mDCs in this model allows for the assessment of in vivo immune-mediated activity of bispecific IgG1 Fcₙᵤₗₗ recombinant antibodies or bispecific IgG1 Fcₙᵤₗₗ u recombinant antibodies conjugated with a TLR8 agonist or STING agonist. Bispecific IgG1 Fcₙᵤₗₗ recombinant antibodies or by bispecific IgG1 Fcₙᵤₗₗ recombinant antibodies conjugated with a TLR8 agonist or STING agonist bearing one of the following target antigen specificities (HER2, CEA, EGFRvIII) and one of the following effector antigen specificities (CD40, DEC205) is injected intraperitoneally immediately prior to tumor injection. As a control, bispecific IgG1 Fcₙᵤₗₗ recombinant antibodies or by bispecific IgG1 Fcₙᵤₗₗ recombinant antibodies conjugated with a TLR8 agonist or STING agonist in which the target antigen binding domain is not directed to antigen on the injected tumor is injected intraperitoneally immediately prior to tumor injection. Tumor growth is measured using calipers twice per week, beginning seven days post tumor cell transfer and ending at study termination, approximately 3 weeks after tumor cell inoculation. As is shown by the results, tumor growth is reduced only when the injected tumor cells express the target antigen specificity of the bispecific IgG1 Fcₙᵤₗₗ recombinant antibodies or of the bispecific IgG1 Fcₙᵤₗₗ recombinant antibodies conjugated with a TLR8 agonist or STING agonist.

### Example 45

### Treatment of p185HER2 Expressing Cancer by Administering a Recombinant Antibody

This example describes treatment of p185HER2 expressing cancer with a recombinant antibody. A human patient is diagnosed with a cancer that expresses the tumor-associated antigen p185HER2, such as breast cancer or gastric cancer. The recombinant antibody is comprised of a target antigen binding domain, wherein the target antigen binding domain specifically binds the tumor-associated antigen p185HER2, and an effector antigen binding domain, wherein the effector antigen binding domain specifically binds CD40 molecules present on an antigen presenting cell, and an Fc comprising domain with one or more amino acid substitutions that reduce the affinity of the Fc domain to an Fc receptor compared to the affinity of the Fc domain to an Fc receptor in the absence of the one or more amino acid substitutions. The Fc comprising domain is attached to the p185HER2 antigen binding domain, and the Fc comprising domain is attached to the CD40 antigen binding domain. Conditional agonism of the CD40 molecules present on the antigen presenting cells that are present in proximity to the p185HER2 expressing tumor cells is induced by the recombinant antibody, thereby activating the antigen presenting cells and promoting an anti-tumor immune response.

### Example 46

### Treatment of CEA Expressing Cancer by Administering a Recombinant Antibody

This example describes treatment of carcinoembryonic antigen (CEA) expressing cancer with a recombinant antibody. A human patient is diagnosed with a cancer that expresses the tumor-associated antigen CEA, such as colorectal cancer. The recombinant antibody is comprised of a target antigen binding domain, wherein the target antigen binding domain specifically binds the tumor-associated antigen CEA, and an effector antigen binding domain, wherein the effector antigen binding domain specifically binds CD40 molecules present on an antigen presenting cell, and an Fc comprising domain with one or more amino acid substitutions that reduce the affinity of the Fc comprising domain to an Fc receptor compared to the affinity of the Fc domain to an Fc receptor in the absence of the one or more amino acid substitutions. The Fc comprising domain is attached to the CEA antigen binding domain, and the Fc comprising domain is attached to the CD40 antigen binding domain. An anti-tumor immune response and allowance of tolerable systemic use of a CD40 agonist at doses that are higher than can be achieved with CD40 agonist molecules alone are induced by the conditional agonism of the CD40 molecules present on the antigen presenting cells that are located in proximity to the CEA expressing tumor cells.

### Example 47

### Treatment of DLL3 Expressing Cancer by Administering a Recombinant Antibody

This example describes treatment of DLL3 expressing cancer with a recombinant antibody. A human patient is diagnosed with a cancer that expresses the tumor-associated antigen DLL3, such as small cell lung cancer. The recombinant antibody is comprised of a target antigen binding domain, wherein the target antigen binding domain specifically binds the tumor-associated antigen DLL3, and an effector antigen binding domain, wherein the effector antigen binding domain specifically binds CD40 molecules present on an antigen presenting cell, and an Fc comprising domain with one or more amino acid substitutions that reduce the affinity of the Fc comprising domain to an Fc receptor compared to the affinity of the Fc comprising domain to an Fc receptor in the absence of the one or more amino acid substitutions. The Fc comprising domain is attached to the DLL3 antigen binding domain, and the Fc comprising domain is attached to the CD40 antigen binding domain. An anti-tumor immune response and allowance of tolerable systemic use of a CD40 agonist at doses that are higher than can be achieved with CD40 agonist molecules alone are induced by the conditional agonism of the CD40 molecules present on the antigen presenting cells that are located in proximity to the DLL3 expressing tumor cells.

### Example 48

### Stability of Recombinant Antibody and Recombinant Antibody Conjugated with an Immunostimulatory Molecule in IgG Depleted Human Serum

This example shows the stability of recombinant antibody and recombinant antibody conjugated with an immune stimulatory molecule in IgG depleted human serum. Stability of recombinant antibody and recombinant antibody conjugated with an immune stimulatory molecule in human serum (IgG depleted) is measured over 96 hours at 37° C. using either a direct HIC-UV analysis approach (Method A) or an affinity capture approach (Method B). Recombinant antibody or recombinant antibody conjugated with an immune stimulatory molecule are spiked in IgG-depleted human serum (BBI solutions # SF142-2) in sterile tubes (75% final serum concentration) and samples are split into 4 aliquots of equal size then transferred to a 37° C. incubator. One of the aliquots of each sample is taken from the incubator at each time-point (T=0h, 24h, 48h, 96h) and the stability of the recombinant antibody or the average immune stimulatory molecule-recombinant antibody ratios (DAR) is recorded.

### Method A: Direct HIC-UV Analysis

At 0, 24, 48 and 96 hours after the beginning of incubation, the recombinant antibody or recombinant antibody conjugated with an immune stimulatory molecule spiked in IgG depleted human serum is analyzed by analytical hydrophobic interaction chromatography (HIC) using a TOSOH TSKgel Butyl-NPR 4.6 mm×35 mm HIC column (TOSOH Bioscience, #14947) connected to a Dionex Ultimate 3000RS HPLC system (ThermoFisher Scientific, Hemel Hemstead, UK). The stability of the recombinant antibody or conjugate is assessed and is found to remain stable at each time point. The average DAR is found to remain stable at each time point. at each time point.

### Method B: Affinity Capture, De-Glycosylation and RP-ESI-MS Analysis

Recombinant antibody or recombinant antibody conjugated with an immune stimulatory molecule is immunocaptured from the IgG depleted human serum using an anti-Human IgG (Fc specific) biotin antibody immobilized on streptavidin beads at 0, 24, 48 and 96 hours after the beginning of incubation. After elution from the beads, the samples are de-glycosylated using agarose-immobilized EndoS (Genovis Inc, USA). The de-glycosylated recombinant antibody conjugated with an immune stimulatory molecule is analyzed by reverse phase chromatography hyphenated to electrospray ionization mass spectrometry (RP-ESI-MS) using an Acquity nano UPLC in line with a Xevo G2S Q-TOF (Waters, Elstree, UK). The separation is performed using an Acquity UPLC online coupled to an ESI-MS mass spectrometer. Mass spectrometric analysis is performed in positive ion mode, scanning from 1000 to 4000 m/z in high mass operating mode. The ion envelope produced by each sample is deconvoluted using the MaxEnt1 algorithm provided within the MassLynx software (Waters, Elstree, UK). The stability of the recombinant antibody is assessed and is found to remain stable at each time point. The average DAR is found to remain stable at each time point.

### Example 49

### Bispecific IgG1 Fcnull Recombinant Antibody Showed Reduction in Binding to the Cellularly Expressed Antigen Presenting Cell Target CD40 Compared to Parental Anti-CD40 Monoclonal Antibody

This example shows that a bispecific IgG1 Fcₙᵤₗₗ recombinant antibody had reduced binding to the antigen presenting cell (APC) target CD40 when binding was measured on whole cells expressing native protein compared to parental anti-CD40 monoclonal antibody.

Anti-HER2-anti-CD40 Fcₙᵤₗₗ antibody was prepared as described in Example 35. Anti-HER2-anti-CD40 IgG1 Fcₙᵤₗₗ recombinant antibody (HER2-CD40 G1ₙᵤₗₗ) bound to CD40 expressed on dendritic cells less efficiently than the parental anti-CD40 monoclonal antibody (SBT-040 G1). Monocytes were isolated from total human peripheral blood mononuclear cells (PBMCs) by magnetic bead-based negative selection. To generate monocyte-derived dendritic cells (moDCs), monocytes were cultured for 7 days in the presence of GM-CSF and IL-4. moDCs were stained with equivalent molar concentrations of either SBT-040 G1 antibody or HER2-CD40 G1ₙᵤₗₗ recombinant antibody followed by incubation with a PE-labeled goat anti-human IgG polyclonal detection antibody. As shown in FIG. 7B, assessment of antibody binding by flow cytometry indicated a binding EC₅₀ for SBT-040 G1 antibody of 2.3 nM compared to a binding EC₅₀ of 33.9 nM for HER2-CD40 G1ₙᵤₗₗ recombinant antibody, and FIG. 7B also shows that HER2-CD40 G1ₙᵤₗₗ recombinant antibody bound to CD40 expressed on dendritic cells less efficiently than the parental anti-CD40 monoclonal antibody, SBT-040G1. Therefore, HER2-CD40 G1ₙᵤₗₗ recombinant antibody binding to surface CD40 on moDCs was decreased compared to SBT-040 G1 antibody.

### Example 50

### Dendritic Cells were Conditionally Activated by Bispecific IgG1 Fcnull Recombinant Antibody when Bound to Tumor Antigen as Measured by Activation Surface Marker

This example shows that expression of the activation surface markers CD54, CD86, CD83, CD80 and MHC II (HLA-DR) were increased on human dendritic cells (DCs) by a bispecific IgG1 Fcₙᵤₗₗ recombinant antibody when bound to tumor antigen.

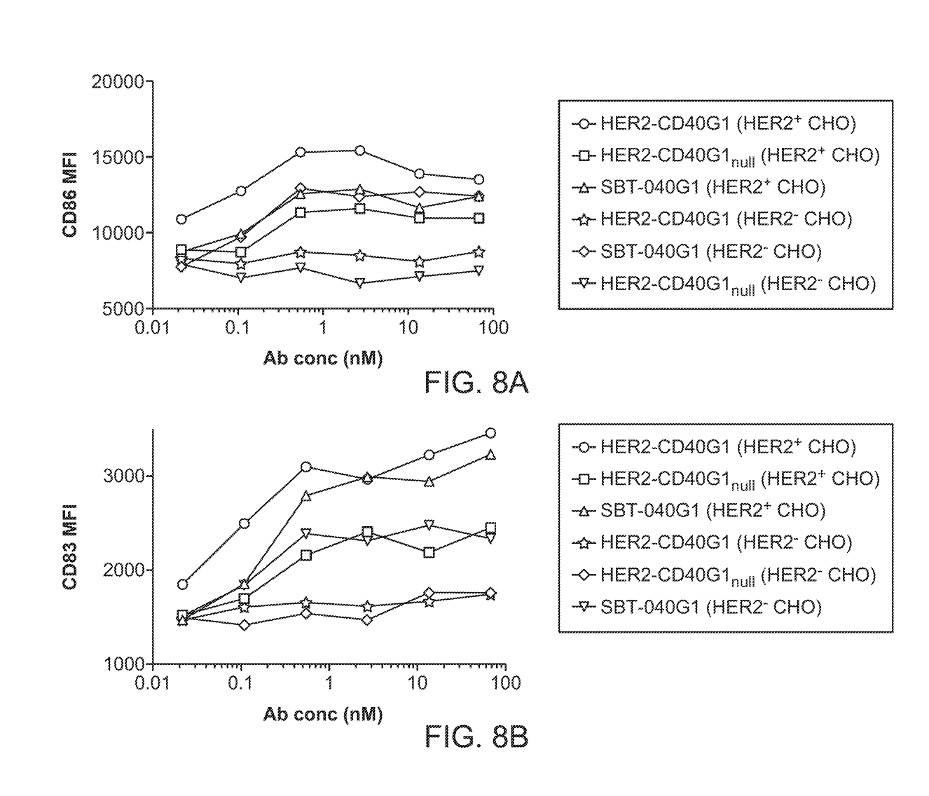

The anti-HER2-anti-CD40 Fcₙᵤₗₗ antibody was prepared as described in Example 35. Anti-HER2-antiCD40 G1 antibody was prepared as described in Example 18. DCs were derived from human peripheral blood mononuclear cells (PBMCs) by isolation of CD14⁺ monocytes followed by culture in RPMI containing 10% fetal calf serum for seven days in complete medium supplemented with 10 ng/mL IL-4 and 100 ng/mL GM-CSF. CHO cells were engineered to overexpress human HER2 using standard transient transfection methods and transiently transfected cells were used in assays 2-3 days after transfection. Dendritic cells were plated with either HER2-expressing CHO cells or parental CHO cells with no HER2 expression at a 2:1 ratio in the presence of titrating concentrations of anti-HER2-anti-CD40 IgG1 Fcₙᵤₗₗ recombinant antibody (HER2-CD40 G1ₙᵤₗₗ) or the parental anti-CD40 monoclonal antibody (SBT-040 G1). After 24 hours, the cells were collected, washed and stained on ice with a collection of commercially available antibodies conjugated to fluorophores directed against CD54, CD86, CD83, CD80 and HLA-DR as per the manufacturer's recommended protocol. After washing to remove unbound antibody-fluor molecules, the stained cells were subjected to FACS analysis using a Celesta flow cytometer (BD Biosciences) with gating on live cells. The output was analyzed by FlowJo v10.2 software (FlowJo LLC). The addition of HER2-CD40 G1ₙᵤₗₗ recombinant antibody to co-cultures containing CHO-HER2 cells resulted in increased DC expression of all activation surface markers. In contrast, activation surface marker expression was not increased above baseline after addition of HER2-CD40 G1ₙᵤₗₗ recombinant antibody to co-cultures containing the CHO parental line without HER2 expression. For the control SBT-040 G1 antibody, co-cultures containing CHO-HER2 cells or CHO parental line without HER2 expression showed similar increased DC expression of activation surface markers, indicating activation surface marker expression induced by SBT-040 G1 was not dependent on cross-linking to tumor antigen. FIGS. 8A & 8B show representative data for CD86 and CD83, respectively. Therefore, DCs were conditionally activated by bispecific IgG1 Fcₙᵤₗₗ recombinant antibodies in the presence of tumor antigen.

### Example 51

### Antibody-Dependent Cellular Cytoxicity was Effectively Mediated by Bispecific Recombinant Antibody Constructs

This example shows that the activation of cell surface Fcγ receptors (FcγRs) is lowered when recombinant antibody is bound to dendritic cells in the absence of tumor antigen. The recombinant antibody is comprised of a first antigen binding domain, wherein the first antigen binding domain specifically binds to the tumor-associated antigen, and a second antigen binding domain, wherein the second antigen binding domain specifically binds to CD40 molecules present on an antigen presenting cell, and an Fc domain with one or more amino acid substitutions that reduce the affinity of the Fc domain to an Fc receptor compared to the affinity of the Fc domain to an Fc receptor in the absence of the one or more amino acid substitutions.

A reporter gene system is used to demonstrate lowered FcγR activation by the recombinant antibody compared to CD40 monoclonal antibody. Stable reporter cell lines for individual FcγRs are produced using the human leukemic T cell line Jurkat. This is done by using cDNAs encoding the FcγRs (and encoding the Fcγ chain for FcγRI and FcγRIIIa), cloning them into pVITRO1-neo-mcs vector (InvivoGen) and then introducing them into cells by electroporation followed by G418 drug selection. Stable individual FcγR expressing lines are created by limit dilution for clones and screening by FACS for cell surface FcγR using commercially available fluor-conjugated anti-FcγR antibodies. Vector pGL4.30[luc2P/NFAT-RE/Hygro] vector (Promega) encoding the reporter for FcγR activation is then introduced into the lines by transfection followed by hygromycin selection to yield stable FcγR+NFAT:LUC reporter cell lines. Next, 2×10⁴ of either myeloid derived dendritic cells generated from human normal peripheral blood mononuclear cells (PBMCs) bearing cell surface CD40 or cells from the human B lymphoblast cell line Daudi bearing cell surface CD40, are added to wells of a 96-well tissue culture dish along with 1×10⁵ Jurkat-FcγR-NFAT:LUC cells with comprising serial dilutions of the recombinant antibody or a monoclonal CD40 antibody. No tumor-associated antigen that binds to the first antigen binding domain is present in the wells. After 5 hours of incubation at 37° C./5% CO₂, FcγR activation is assessed by measuring luciferase activity using the ONE-Glo Luciferase Assay System (Promega) and the EnSpire Multimode Plate Reader (PerkinElmer). Diminished luciferase activity is found over the dose titration with the recombinant antibody, which demonstrates lower FcγR activation of the recombinant antibody.

### Example 52

### Binding of Cell Surface Fcγ Receptors is Lowered when Recombinant Antibody is Bound to Dendritic Cells in the Absence of Tumor Antigen

This example shows that the binding of cell surface Fcγ receptors (FcγRs) is lowered when recombinant antibody is bound to dendritic cells in the absence of tumor antigen. The recombinant antibody is comprised of a target antigen binding domain, wherein the target antigen binding domain specifically binds to the tumor-associated antigen, and an effector antigen binding domain, wherein the effector antigen binding domain specifically binds to CD40 molecules present on an antigen presenting cell, and an Fc comprising domain with one or more amino acid substitutions that reduce the affinity of the Fc comprising domain to an Fc receptor compared to the affinity of the Fc comprising domain to an Fc receptor in the absence of the one or more amino acid substitutions.

A flow cytometry-based antibody-mediated cell bridging assay is used to assess FcγR binding. Stable Jurkat cell lines expressing individual FcγRs are produced using cDNAs encoding the FcγRs (and encoding the Fcγ chain for FcγRI and FcγRIIIa) cloned into pVITRO1-neo-mcs vector (InvivoGen) that are introduced into cells by electroporation followed by G418 drug selection. Stable individual FcγR expressing lines are created by limiting dilution of clones and screening by fluorescence activated cell sorting (FACS) for cell surface FcγR using commercially available fluor-conjugated anti-FcγR antibodies. Next, either myeloid derived dendritic cells generated from human normal donor peripheral blood mononuclear cells (PBMCs) bearing cell surface CD40 or the human B lymphoblast cell line Daudi bearing cell surface CD40 are labelled with calcein AM (Ebioscience). The Jurkat FcγR clones are labelled with calcein violet 450 AM (Ebioscience). Then, 3×10⁴ CD40⁺ cells, 3×10⁵ Jurkat FcγR cells, and serial dilutions of either the recombinant antibody or a monoclonal CD40 antibody are added to wells of a 96 well tissue culture plate, incubated at 37° C./5% CO₂ for 30 minutes, followed by FACS analysis using a BD Celesta flow cytometer with FACS Diva software. No tumor-associated antigen that binds to the first antigen binding domain is present in the wells. Blocking the bridging by including commercially available anti-FcγR antibodies during the 30 minute assay incubation is used a control for FcγR dependency on bridging. The number of events that are recorded as double calcein/calcein violet as compared to control as analyzed with FlowJo software are evidence of the extent of antibody mediated cell bridging. The extent of cell bridging is diminished for the recombinant antibody as compared to the monoclonal CD40 antibody, demonstrating that the capacity to bind to FcγRs when bound to antigen presenting cell is lowered with recombinant antibody in the absence of the tumor-associated antigen of the first antigen binding domain.

### Example 53

### Dendritic Cells were Conditionally Activated by Bispecific Recombinant Antibody Constructs when Bound to Tumor Antigen as Measured by Activation Surface Markers

This example shows that expression of the activation surface markers CD54, CD86, CD83, CD80, and MHC II (HLA-DR) are increased on human dendritic cells (DCs) by a bispecific tumor targeting antibody construct bound to tumor antigen. This example also illustrates that this increased expression of activation surface markers on DCs was dependent upon the agonism of CD40 and agonism of Fc receptors.

The anti-HER2-anti-CD40 Fcₙᵤₗₗ antibody was prepared as described in Example 35. Anti-HER2-antiCD40 G1 antibody was prepared as described in Example 18. DCs were derived from human peripheral blood mononuclear cells (PBMCs) by isolation of CD14+ monocytes followed by culture in RPMI containing 10% fetal calf serum for seven days in complete medium supplemented with 10 ng/mL IL-4 and 100 ng/mL GM-CSF. CHO cells were engineered to overexpress human HER2 using standard transient transfection methods and transiently transfected cells were used in assays 2-3 days after transfection. DCs were plated with either HER2-expressing CHO cells or parental CHO cells with no HER2 expression at a 2:1 ratio in the presence of titrating concentrations of bispecific anti-HER2-anti-CD40 IgG1 (HER2-CD40G1) antibody construct, bispecific anti-HER2-anti-CD40 IgG1 Fc null antibody construct (HER2-CD40G1ₙᵤₗₗ), or the parental anti-CD40 monoclonal antibody (SBT-040G1). After 24 hours, the cells were collected, washed and stained on ice with a collection of commercially available antibodies conjugated to fluorophores directed against CD54, CD86, CD83, CD80, and HLA-DR as per the manufacturer's recommended protocol. After washing to remove unbound antibody-fluor molecules, the stained cells were subjected to FACS analysis using a Celesta flow cytometer (BD Biosciences) with gating on live cells. The output was analyzed by FlowJo v10.2 software (FlowJo LLC). The addition of HER2-CD40G1 antibody construct to co-cultures containing HER2⁺ CHO cells resulted in increased DC expression of all activation surface markers. In contrast, activation surface marker expression was not increased above baseline after addition of HER2-CD40G1 antibody to co-cultures containing the CHO parental line without HER2 expression (HER2-CHO cells). For the control SBT-040G1 antibody, co-cultures containing HER2⁺ CHO cells or HER2-CHO cells showed similar increased DC expression of activation surface markers, indicating activation surface marker expression induced by SBT-040G1 was not dependent on cross-linking to tumor antigen. Additionally, the HER2-CD40G1ₙᵤₗₗ antibody construct increased activation marker expression significantly over baseline, but the increased expression was reduced compared to the HER2-CD40G1 antibody construct. FIGS. 8A & 8B show representative data for CD86 and CD83, respectively. These data demonstrate that CD40 agonism and Fc receptor agonism both contributed to the increase in activation surface marker expression on DCs observed with a bispecific recombinant antibody constructs in the presence of tumor antigen. Therefore, DCs were conditionally activated by bispecific recombinant antibody construct in the presence of tumor antigen.

### Example 54

### Antibody-Dependent Cellular Cytoxicity was Effectively Mediated by Bispecific Recombinant Antibody Constructs

This example shows that antibody-dependent cellular cytotoxicity (ADCC) was effectively mediated by bispecific recombinant antibody constructs.

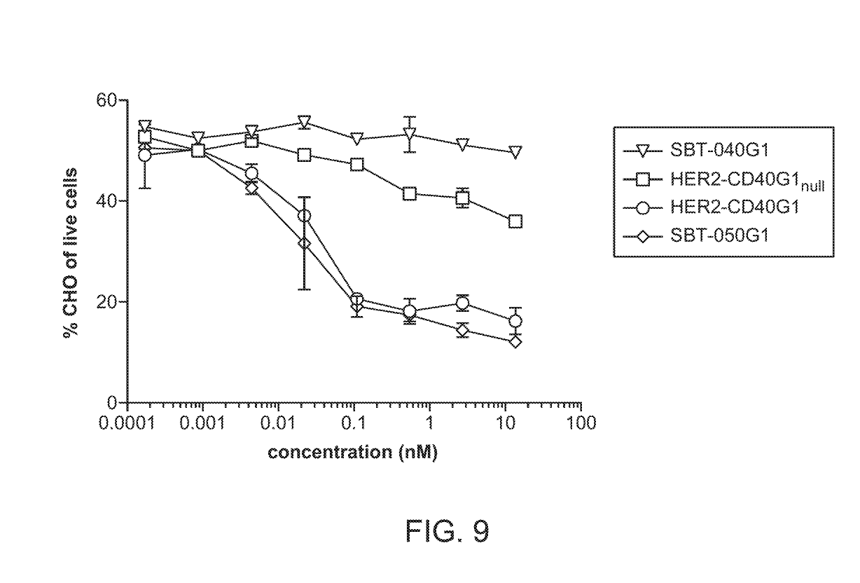

The anti-HER2-anti-CD40 Fcₙᵤₗₗ antibody was prepared as described in Example 35. Anti-HER2-antiCD40 G1 antibody was prepared as described in Example 18. The ability of the bispecific anti-HER2-anti-CD40 IgG1 (HER2-CD40G1) recombinant antibody construct to mediate ADCC as effectively as the anti-HER2 parental monoclonal antibody (SBT-050G1) was shown using a macrophage based in vitro assay. Monocytes were isolated from human peripheral blood mononuclear cells (PBMCs) using magnetic bead-based negative selection and cultured 7 days in the presence of GM-CSF to generate macrophages. A CHO cell line was engineered to overexpress human HER2 using standard transient transfection methods and used in assays 2 days after transfection. Macrophage cells were plated with HER2-expressing CHO cells at a 2:1 ratio in the presence of titrating concentrations of HER2-CD40G1 recombinant antibody constructs, anti-HER2-anti-CD40 IgG1 Fc null recombinant antibody constructs (HER2-CD40G1ₙᵤₗₗ), the parental anti-CD40 monoclonal antibodies (SBT-040G1) or SBT-050G1 antibodies. After 24 hours, CHO frequency was assessed by flow cytometry as a readout of ADCC activity. A reduction in CHO frequency was indicated by engulfment of the macrophages. Referring to FIG. 9, the HER2-CD40G1 recombinant antibody construct mediated equivalent ADCC activity to SBT-050G1 antibody. SBT-040G1 was unable to bind to the CHO target cells and did not facilitate ADCC. Treatment with the HER-CD40G1ₙᵤₗₗ recombinant antibody construct, which was engineered to no longer bind to Fc receptors, resulted in decreased ADCC relative to HER2-CD40G1 recombinant antibody construct. Therefore, the IgG1 Fc domain in the recombinant antibody construct was required to mediate the ADCC function.

### Example 55

### TNFα Production by PBMCs was Induced by Immune-Stimulatory Conjugates

This example shows that immune-stimulatory conjugates can increase production of a pro-inflammatory cytokine, TNFα, by PBMCs in the presence of tumor cells expressing a target tumor antigen.

Anti-HER2-antiCD40 G1 antibody was prepared as described in Example 18. Pertuzumab was used as the HER2 G1WT antibody. Conjugates were prepared as generally described in Example 15. PBMCs were isolated from human blood as described above. Briefly, PBMCs were isolated by Ficoll gradient centrifugation, resuspended in RPMI, and plated in 96-well flat bottom microtiter plates (125,000/well). Antigen-expressing tumor cells were then added (25,000/well) along with titrating concentrations of conjugates or unconjugated parental antibodies as controls. After overnight culture, supernatants were harvested, and TNFα levels were determined by AlphaLISA.

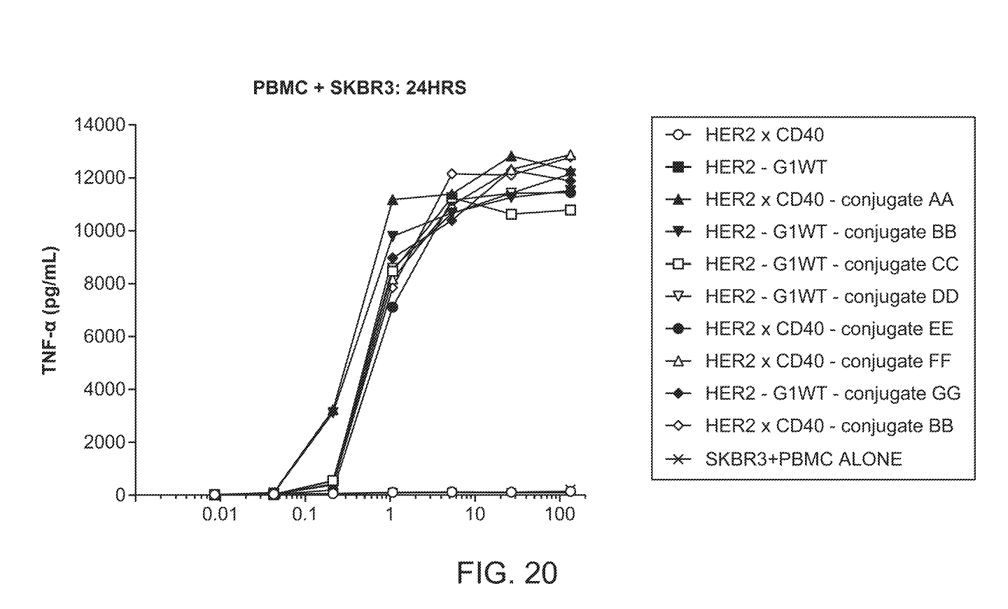

Referring to FIG. 20, SKBR3 tumor cells were added to PBMCs, prepared as described above. SKBR3 tumor cells express the tumor antigen HER2. Anti-HER2 TLR8 benzazepine agonist conjugates, either monospecific or bispecific with CD40, and conjugated to various TLR8 benzazepine agonists were added. The immune-stimulatory compound-linkers in the conjugates used in the study are identified in TABLES 30 and 31.

TABLE 30
Exemplary Compound-Linkers
Structure

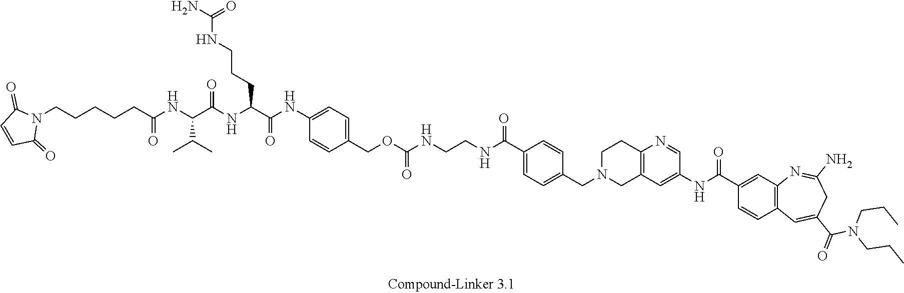 [SMILES: CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

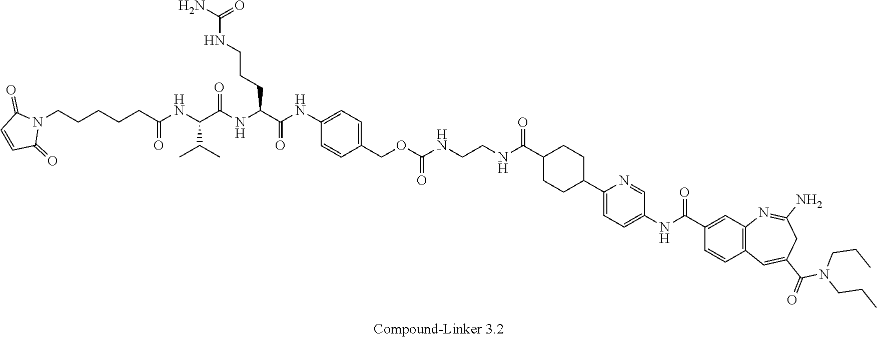 [SMILES: CCCN(CCC)C(=O)C1=Cc2ccc(C(=O)Nc3ccc(C4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1]

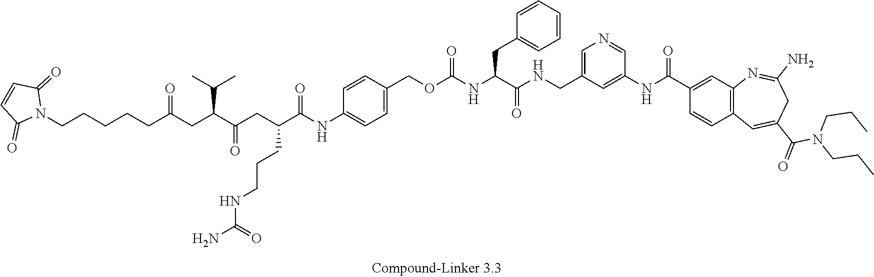 [SMILES: CCCN(CCC)C(=O)C1=Cc2ccc(C(=O)Nc3cncc(CNC(=O)[C@H](Cc4ccccc4)NC(=O)OCc4ccc(NC(=O)[C@H](CCCNC(N)=O)CC(=O)[C@@H](CC(=O)CCCCCN5C(=O)C=CC5=O)C(C)C)cc4)c3)cc2N=C(N)C1]

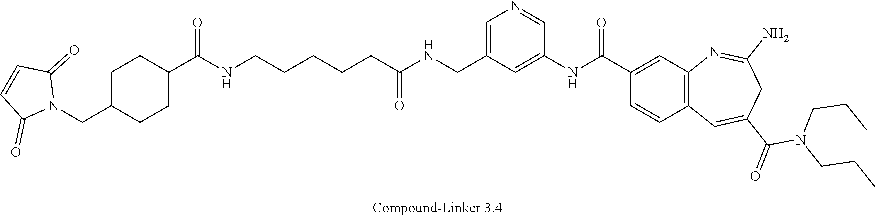 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1]

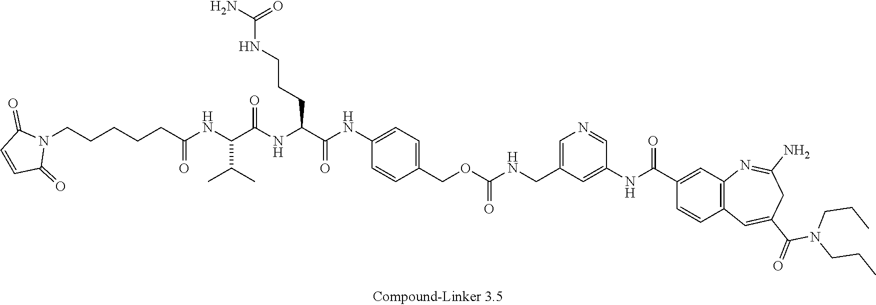 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

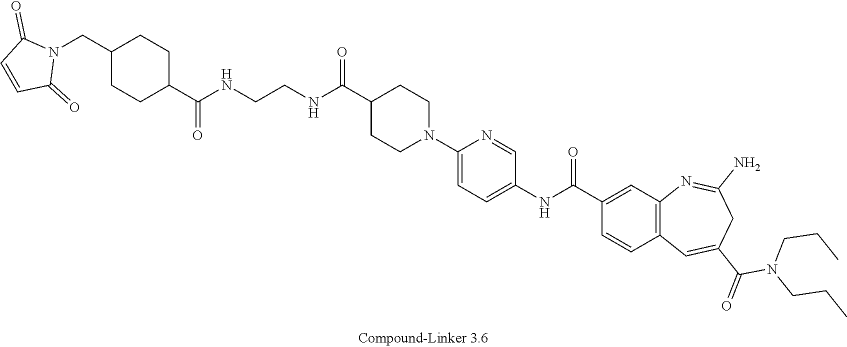 [SMILES: CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1]

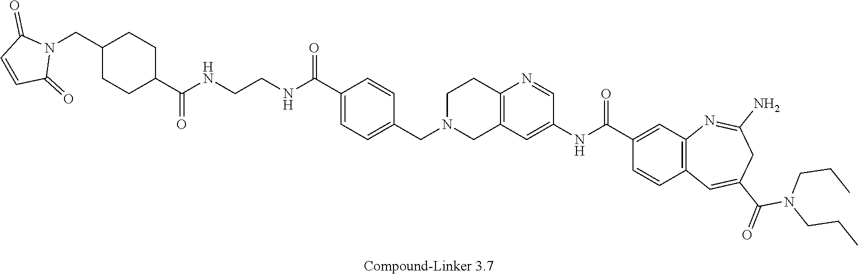 [SMILES: CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

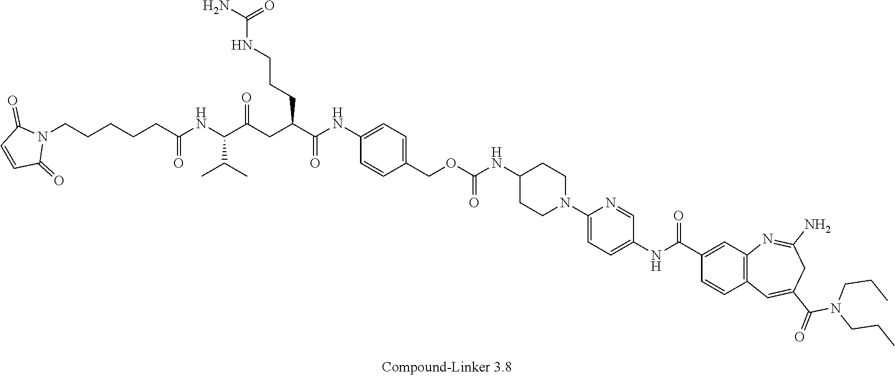 [SMILES: CCCN(CCC)C(=O)C1=Cc2ccc(C(=O)Nc3ccc(N4CCC(NC(=O)OCc5ccc(NC(=O)[C@H](CCCNC(N)=O)CC(=O)[C@@H](NC(=O)CCCCCN6C(=O)C=CC6=O)C(C)C)cc5)CC4)nc3)cc2N=C(N)C1]

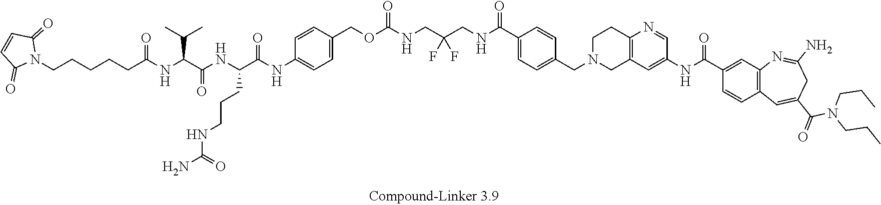 [SMILES: CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

TABLE 31
Exemplary Conjugates and Compound-Linkers Thereof

| Conjugate | Compound-Linker |
|---|---|
| Conjugate 1, Conjugate DD, | Compound-Linker 3.1 |
| Conjugate FF, Conjugate C | |
| Conjugate 2, Conjugate CC, | Compound-Linker 3.2 |
| Conjugate EE, Conjugate B | |
| Conjugate 3, Conjugate F | Compound-Linker 3.3 |
| Conjugate 6, conjugate, conjugate H | Compound-Linker 3.4 |
| conjugate I, TLR 8 Agonist | |
| Conjugate AA, Conjugate BB, | Compound-Linker 3.5 |
| Conjugate A | |
| Conjugate GG, Conjugate BB | Compound-Linker 3.6 |
| Conjugate D | Compound-Linker 3.7 |
| Conjugate E | Compound-Linker 3.8 |
| Conjugate G | Compound-Linker 3.9 |

TNFα production was measured after 24 hours. All of the conjugates were active, stimulating production of TNFα in a dose-dependent manner. In contrast, unconjugated HER2 antibody (HER2 G1WT) and bispecific HER2×CD40 antibody (HER2×CD40) did not stimulate TNFα production. (Both controls are adjacent the X-axis.)

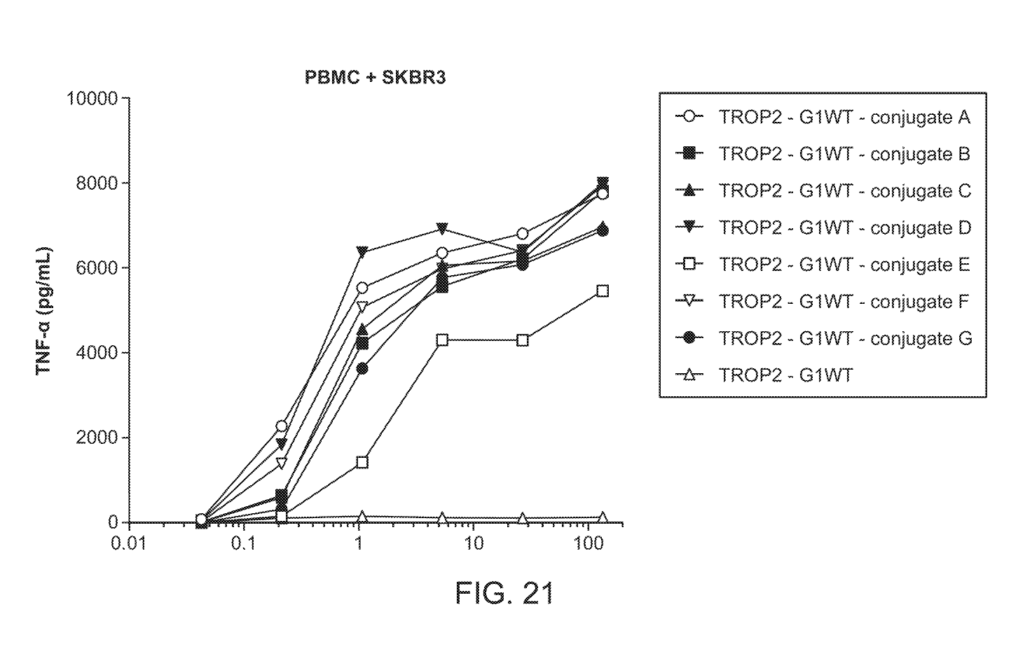

Referring to FIG. 21, a similar study was performed using anti-TROP2 conjugates. TROP2 antibodies conjugated to TLR8 benzazepine agonists stimulated the production of TNFα in the presence of PBMCs, while the unconjugated control antibody did not.

### Example 56

### TNFα Production by Monocytes was Induced by Immune Stimulatory Conjugates

This example shows that monospecific and bispecific immune-stimulatory conjugates can increase the production of a pro-inflammatory cytokine, TNFα, by monocytes in the presence of tumor cells a targeted tumor antigen.

Monocytes were prepared as follows: PBMCs were isolated from normal human blood using Ficoll purification, and monocytes were enriched from the PBMCs using Stem Cell Technologies Human Monocyte without CD16 Depletion Negative Selection Kits according to the manufacturer's instructions. Monocytes were then slowly frozen and stored in liquid nitrogen. Prior to assay monocytes were thawed and rested overnight at 37° C. in 5% CO₂ in assay media (RPMI 1640 media supplemented with 10% FBS, 50 μg/mL Penicillin, 50 U/mL Streptomycin, 1 mM HEPES, 1× non-essential amino acids, 0.1 mM sodium pyruvate). The following day, monocytes were plated at 4×10⁴ cell/well with or without tumor cells at 4×10⁴ cell/well in 96-well flat bottom plates in assay media described above. After overnight culture, supernatants were harvested, and TNFα levels were determined by AlphaLISA.

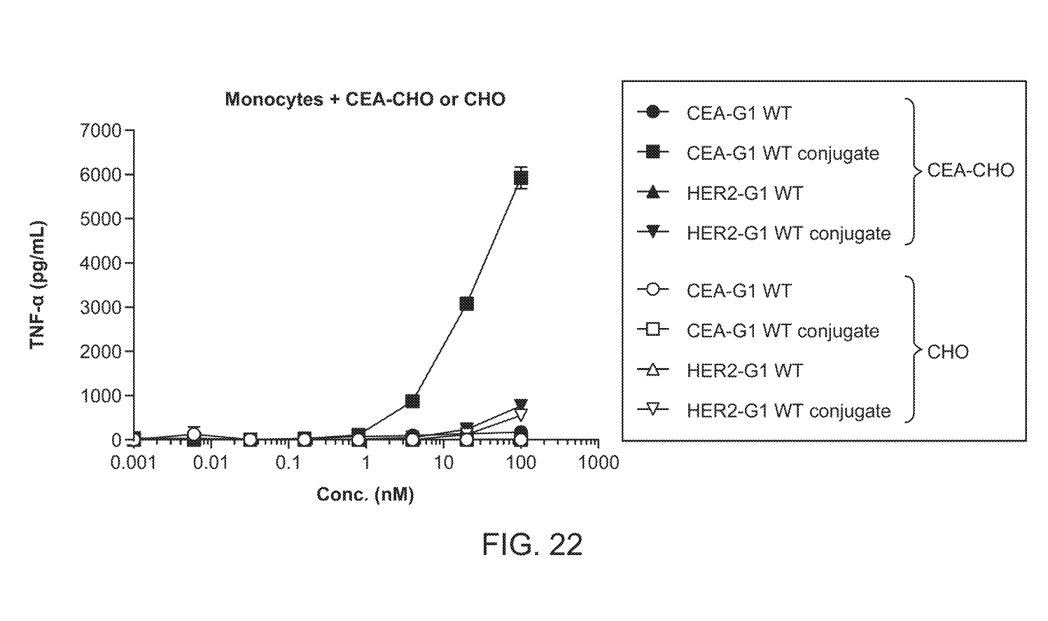

Referring to FIG. 22, the activity of a CEA TLR8 benzazepine agonist conjugate on CEA-expressing CHO cells and on control non-CEA expressing cells was determined. CEA antibody (CEA-G1 WT), HER2 antibody (HER2-G1 WT antibody) and HER2 conjugate (HER2-G1 WT conjugate) were used as controls. The immune-stimulatory compound-linkers in the conjugates used in the study are identified in TABLES 30 and 31. Referring to FIG. 22, only the CEA conjugate exhibited activity, as measured by TNFα production, in the presence of monocytes and tumor cells. The antibodies and conjugates did not stimulate any TNFα production in the presence of CHO cells not expressing detectable CEA. The specificity of the conjugate for its targeted antigen is shown by this example.

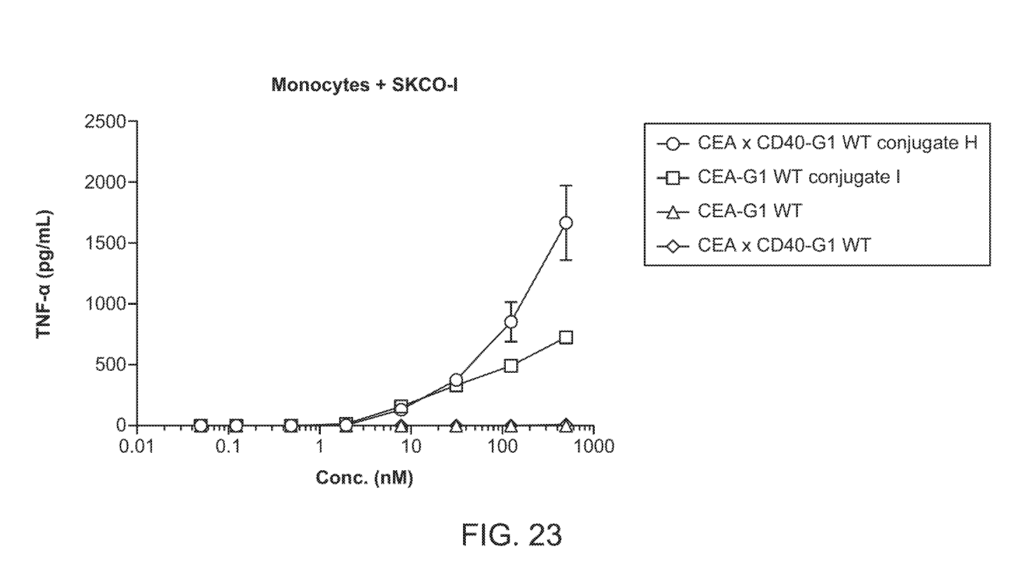

Referring to FIG. 23, SKCO-1 tumor cells were added to monocytes, prepared as described above. SKCO-1 cells express the tumor antigen CEA. Anti-CEA TLR8 benzazepine agonist conjugates, either monospecific or bispecific with CD40, and conjugated to a TLR8 benzazepine agonist, were added. The immune-stimulatory compound-linkers in the conjugates used in the study are identified in TABLES 30 and 31. Anti-CEA×CD40 bispecific antibody was prepared by attaching a CD40 scFv to the C-terminal end of the anti-CEA antibody heavy chain. TNFα production was measured after 24 hours. Both conjugates were active, stimulating production of TNFα in a dose-dependent manner. Notably the bispecific CEA×CD40 conjugate was more active than the monospecific conjugate. In contrast, unconjugated CEA antibody (CEA-G1 WT) and bispecific CEA×CD40 antibody (CEA×CD40) did not stimulate TNFα production.

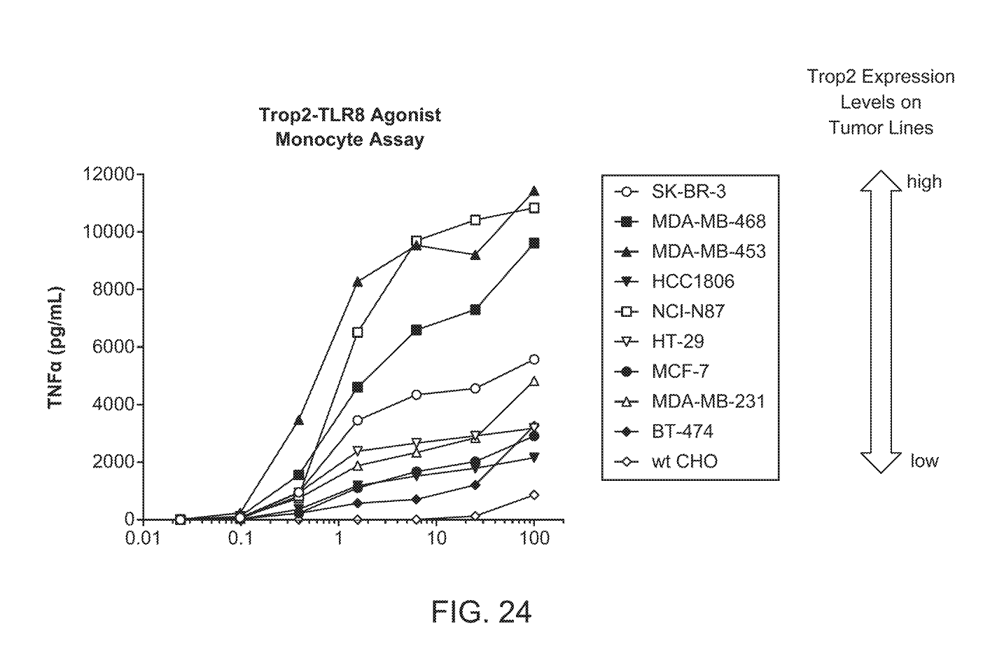

Referring to FIG. 24, another study was performed using an anti-TROP2 conjugate and ten different cell lines expressing varying levels of TROP2. The TROP2 antibody (sacituzumab) was conjugated with a TRL8 benzazepine agonist-linker as generally described in Example 15. The immune-stimulatory compound-linkers in the conjugates used in the study are identified in TABLES 30 and 31. As shown in FIG. 24, the activity of the TROP2-TLR8 benzazepine agonist conjugates, as measured by TNFα production, varied in the dose-dependent manner and generally in accordance with the level of TROP2 expression by the cell line. The control cell line, wt CHO, does not express detectable level of TROP2 and little TNFα production was detected.

### Example 57

### TNFα Production by Macrophages was Induced by Immune Stimulatory Conjugates

This example shows that immune-stimulatory conjugates can increase the production of a pro-inflammatory cytokine, TNFα, by macrophages in the presence of tumor cells expressing the targeted antigen.

Macrophages were generated as follows: Monocytes were isolated from human peripheral blood mononuclear cells (PBMCs) using magnetic bead-based negative selection and cultured 7 days in the presence of GM-CSF to generate macrophages. Macrophages were plated in 96-well flat bottom microtiter plates (40,000/well). Antigen-expressing tumor cells were then added (40,000/well) along with titrating concentrations of conjugates or unconjugated parental antibodies as controls. After overnight culture, supernatants were harvested, and TNFα levels were determined by AlphaLISA.

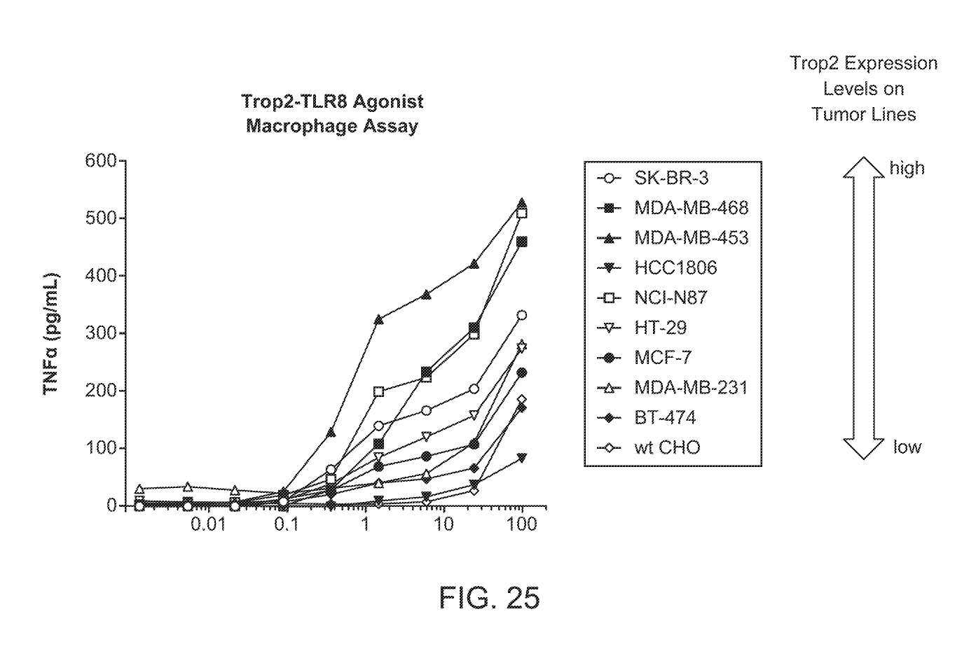

Referring to FIG. 25, a study was performed using an anti-TROP2 conjugate and ten different cell lines expressing varying levels of TROP2. The TROP2 antibody (sacituzumab) was conjugated with a TRL8 benzazepine agonist-linker as generally described in Example 15. The immune-stimulatory compound-linkers in the conjugates used in the study are identified in TABLES 30 and 31. As shown in FIG. 25, the activity of the TROP2-TLR8 benzazepine agonist conjugates, as measured by TNFα production, varied in the dose-dependent manner and generally in accordance with the level of TROP2 expression by the cell line. The control cell line, wt CHO, does not express detectable level of TROP2 and little TNFα production was detected.

### Example 58

### Bispecific Immunostimulatory Conjugates Increase Monocyte-Derived Dendritic Cell Cytokine Release

This example illustrates that TLR8 bispecific antibody conjugates increase activation of monocyte-derived dendritic cells, as shown by IL-12 production and that addition of a CD40 binding domain to a Her2 antibody increases the potency of the conjugate.

PBMCs were isolated from normal human leukapheresis product using Ficoll purification, and monocytes were enriched from PBMC using Stem Cell Technologies Human Monocyte without CD16 Depletion Negative Selection Kits according to the manufacturer's instructions. Monocytes were frozen and stored in liquid nitrogen. Seven days prior to assay, monocytes were thawed and differentiated into monocyte derived dendritic cells (mDC). For differentiation, monocytes were plated at a concentration of 1×10⁶ monocytes/mL in X-Vivo15 media supplemented with 10% fetal bovine serum (FBS), 100 ng/mL recombinant human GM-CSF and 10 ng/mL recombinant human IL-4 and incubated at 37° C. in 5% CO₂. On day 4 of differentiation, half of the media was removed and replaced with fresh X-Vivo15 media supplemented with 10% fetal bovine serum (FBS), 100 ng/mL recombinant human GM-CSF and 10 ng/mL recombinant human IL-4 and returned to 37° C. in 5% CO₂.

mDC were lifted and re-plated on day 7 of differentiation at 4×10⁴ cell/well with 4×10⁴ cell/well Her2 transfected CHO cells or wild type CHO cells in 96-well flat bottom plates in assay media (RPMI 1640 base media supplemented with 10% FBS, 50 μg/mL Penicillin, 50 U/mL Streptomycin, 1 mM HEPES, 1× non-essential amino acids, 0.1 mM sodium pyruvate). Antibodies and antibody conjugates were diluted in assay media and added to co-cultures at final concentrations starting at 667 nM and diluted 5-fold to 0.213 nM in 100 μL/well total volume. Co-cultures were incubated for 24 hours at 37° C. in 5% CO₂ before cell culture supernatants were harvested. IL-12p40 production was assessed in culture supernatants using the Biolegend Human IL-12/IL-23 p40 ELISA Kit according to the manufacturer's instructions. Plates were read using a Perkin Elmer Envision plate reader.

Bispecific HER2×CD40 recombinant antibody was prepared as described in Example 18. Bispecific HER2×CD40 recombinant antibody conjugate and Her2 recombinant antibody conjugate were prepared by conjugation of a HER2×CD40 bispecific antibody and a HER2 monoclonal antibody to a TLR8 benzazepine agonist and a linker, as generally described in Example 15. The average drug loading was about 4.

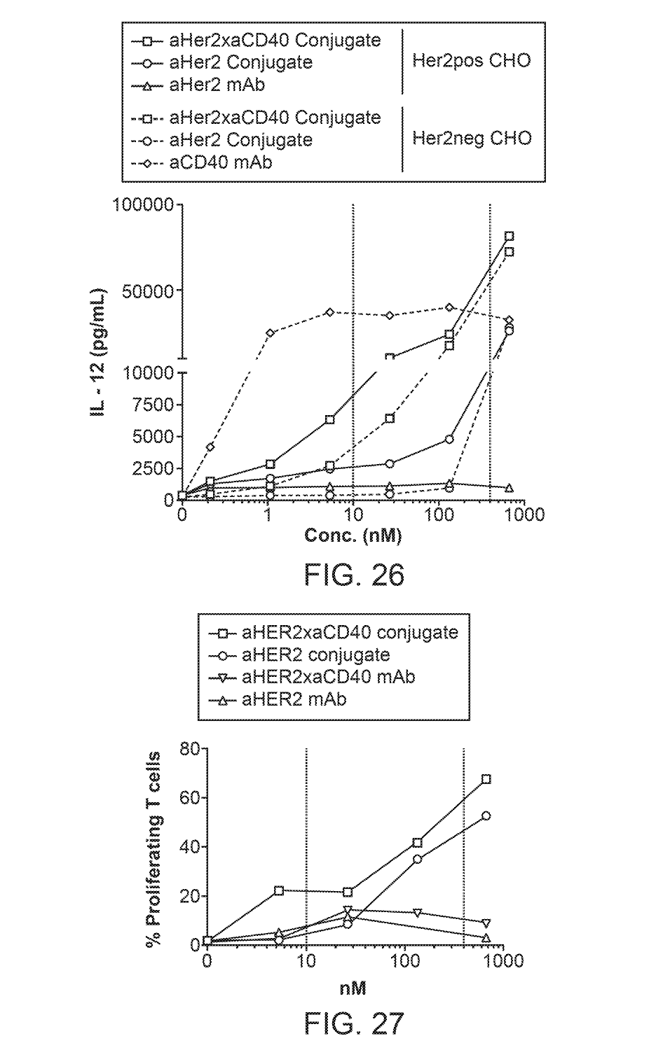

Referring to FIG. 26, a bispecific HER2×CD40 recombinant antibody conjugate was able to stimulate IL-12 production by mDCs in the presence of Her2 positive CHO cells. The bispecific HER2×CD40 recombinant antibody conjugate stimulated less IL-12 production in the absence of Her2 positive CHO cells (i.e., in the presence of HER2 negative CHO cells), showing both binding domains contribute to activity. The HER2 antibody conjugate stimulated less IL-12 production than the bispecific HER2×CD40 conjugate, showing the contribution of the second CD40 binding domain. The control HER2 antibody alone stimulated little IL-12 production by comparison.

### Example 59

### Bispecific and Monospecific Immune Stimulatory Conjugates Increase the Ability of Monocyte-Derived Dendritic Cells to Stimulate T Cells

This example illustrates that TLR8 antibody conjugates can increase activation of monocyte-derived dendritic cells, as shown by increased activation of T cells, and that a bispecific recombinant antibody having a CD40 binding domain is more potent than a monospecific recombinant antibody.

Bispecific HER2×CD40 recombinant antibody conjugate and the HER2 monospecific conjugate were prepared as described in Example 58. To measure T cell stimulation, DCs were generated by culturing human monocytes in the presence of GM-CSF and IL-4. On Day 7 of culture, DCs were lifted and re-plated with HER2-expressing tumor cells in the presence of titrating concentrations of the monoclonal antibody or antibody conjugates. After 24 hours of culture, DCs were lifted and combined with mismatched, proliferation dye-labeled T cells. After 7 days of culture, cells were harvested and stained with CD3-PerCP/Cy5.5. Dye dilution as a measure of T cell proliferation was then assessed by flow cytometry.

Referring to FIG. 27, the bispecific HER2×CD40 recombinant antibody conjugate and the HER2 monospecific conjugate showed increased stimulation of T cell proliferation in the presence of HER2 positive tumor cells as compared to the unconjugated antibodies.

### Example 60

### Bispecific Antibody Conjugate with a CD40 Binding Domain Potentiates CD40 Agonism on B Cells

This example illustrates that human B cells were activated by a HER2×CD40 bispecific antibody conjugate, that the bispecific antibody conjugate potentiated CD40L agonism and that activation was increased by Fc domain-mediated cross-linking.

Human B cells were isolated from fresh PBMCs by magnetic bead enrichment and plated in 96-well flat bottom microtiter plates (50,000/well). HER2-expressing tumor cells were then added (50,000/well) along with titrating concentrations of antibody conjugates or unconjugated monoclonal antibodies. In some studies, sCD40L or anti-human F(ab′)₂ was added to cultures. Following overnight culture, B cell expression of CD86 was assessed by flow cytometry.

Bispecific HER2×CD40 recombinant antibody conjugate and HER2 recombinant antibody conjugate were prepared by conjugation of HER2×CD40 bispecific and a HER2 monoclonal antibody to a TLR8 benzazepine agonist and a linker. Bispecific HER2×CD40 recombinant antibody conjugate and the HER2 monospecific conjugate were prepared as described in Example 58. The average drug loading was about 4.

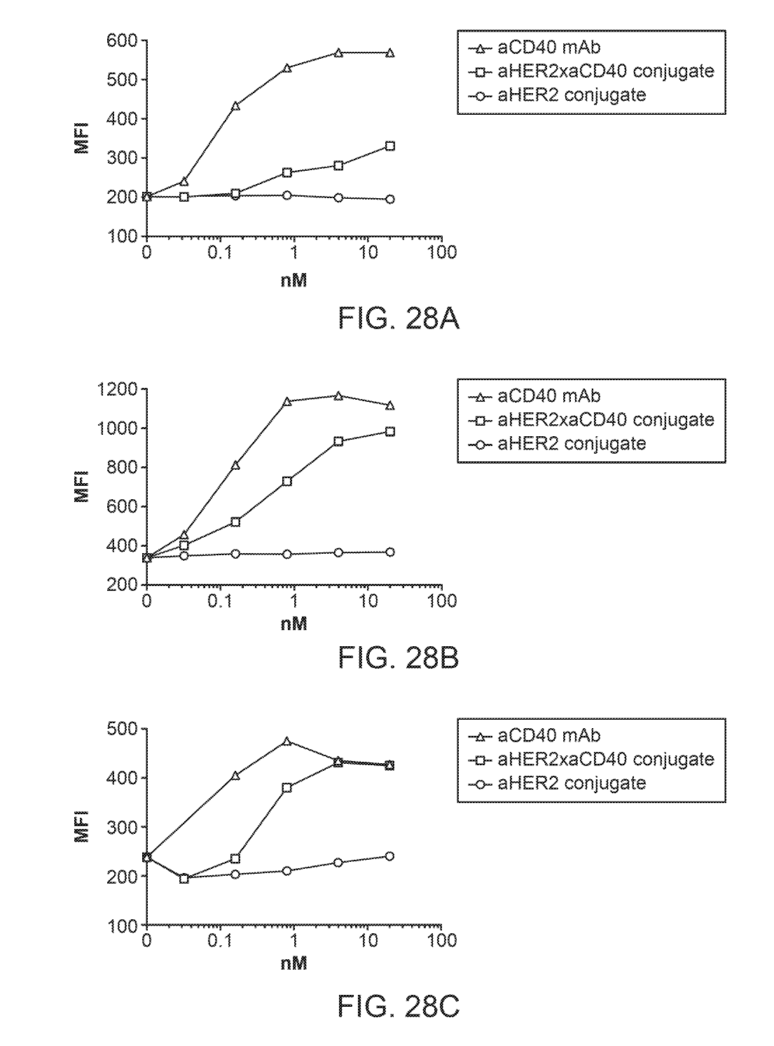

Referring to FIG. 28A, a bispecific HER2×CD40 recombinant antibody conjugate increased expression of CD86 activation marker by primary B cells as compared to a monospecific HER2 recombinant antibody conjugate. CD40 monoclonal antibody was used as a positive control.

Referring to FIG. 28B, addition of CD40L increased activation (CD86 expression) of primary B cells as the amount of bispecific HER2×CD40 recombinant antibody conjugate increased. In contrast, CD86 expression was unchanged in the presence of CD40L and increasing amounts of HER2 monoclonal antibody conjugate.

Referring to FIG. 28C, addition of anti-human F(ab′)₂ crosslinking antibody increased activation (CD86 expression) with increasing amounts of bispecific HER2×CD40 recombinant antibody conjugate by Fc-medicated crosslinking. In contrast, CD86 expression was largely unchanged in the presence of anti-human F(ab′)₂ crosslinking antibody.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A recombinant bispecific antibody, comprising:
a) a target antigen binding domain, wherein the target antigen binding domain specifically binds to a tumor associated antigen;
b) an effector antigen binding domain, wherein the effector antigen binding domain specifically binds to an antigen on an antigen presenting cell and wherein the antigen is a molecule on the antigen presenting cell;
c) an Fc comprising domain; and
d) an immune-stimulatory compound attached to the recombinant bispecific antibody by a linker;
wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

2. A recombinant bispecific antibody, comprising:
a) a target antigen binding domain, wherein the target antigen binding domain specifically binds to a tumor associated antigen;
b) an effector antigen binding domain, wherein the effector antigen binding domain specifically binds to an antigen on an antigen presenting cell and is an antibody antigen binding domain, wherein the antigen is a molecule on the antigen presenting cell; and
c) a domain comprising an Fc region;
wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

3. A recombinant bispecific antibody, comprising:
a) a target antigen binding domain, wherein the target antigen binding domain specifically binds to a tumor associated antigen;
b) an effector antigen binding domain, wherein the effector antigen binding domain specifically binds to an antigen on an antigen presenting cell and is an antibody antigen binding domain, wherein the antigen is a molecule on the antigen presenting cell; and
c) a domain comprising an Fc region;
wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

4. A recombinant bispecific antibody, comprising:
a) a target antigen binding domain, wherein the target antigen binding domain specifically binds to a tumor associated antigen;
b) an effector antigen binding domain, wherein the effector antigen binding domain specifically binds to an antigen on an antigen presenting cell and wherein the antigen is a molecule on the antigen presenting cell; and
c) an Fc comprising domain; and
d) an immune-stimulatory compound attached to the recombinant bispecific antibody by a linker;
wherein the recombinant bispecific antibody induces greater immune cell activation in the presence of cells having cell surface tumor associated antigen and antigen presenting cells having cell surface antigen as compared to immune cell activation in the absence of cells having cell surface tumor associated antigen.

5. The recombinant bispecific antibody of any one of claims 1-4, wherein the immune cell activation is measured by a cytokine release assay.

6. The recombinant bispecific antibody of any one of claims 1, 2, and 5, wherein the immune cell activation by the recombinant bispecific antibody when the recombinant bispecific antibody is bound to the tumor associated antigen and to the antigen on the antigen presenting cell is at least two times, five times, or ten times greater than immune activation by the recombinant bispecific antibody when the recombinant bispecific antibody is bound to the antigen on the antigen presenting cell but not to the tumor associated antigen as measured by the cytokine release assay.

7. The recombinant bispecific antibody of any one of claims 3-5, wherein the immune cell activation by the recombinant bispecific antibody in the presence of cells having cell surface tumor associated antigen and antigen presenting cells having cell surface antigen is at least two times, five times, or ten times greater than immune cell activation by the recombinant bispecific antibody in the absence of the cells having cell surface tumor associated antigen as measured by the cytokine release assay.

8. The recombinant bispecific antibody of any one of claims 1-7, wherein the immune cell activation comprises an increase in one or more of:
a) a secretion of one or more cytokines as measured by the cytokine release assay,
b) a secretion of one or more chemokines as measured by an ELISA immunoassay,
c) an expression level of one or more cell surface proteins associated with immune stimulation as measured by FACS, and
d) an activity of one or more immune cell functions.

9. The recombinant bispecific antibody of claim 8, wherein the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay.

10. The recombinant bispecific antibody of claim 9, wherein the recombinant bispecific antibody induces tumor-cell directed antibody-dependent cell-mediated cytotoxicity.

11. The recombinant bispecific antibody of any one of claims 1-10, wherein the Fc comprising domain has one or more amino acid substitutions that decrease the binding affinity to one or more Fcγ receptors as compared to a wild-type Fc comprising domain.

12. The recombinant bispecific antibody of any one of claims 1-11, wherein the effector antigen binding domain has an increased binding affinity to the antigen on the antigen presenting cell as compared to the binding affinity of the effector antigen binding domain of an antibody that lacks the target antigen binding domain.

13. The recombinant bispecific antibody of any one of claims 1-12, wherein a K_{d} of the binding affinity of the effector antigen binding domain of the recombinant bispecific antibody to the antigen on the antigen presenting cell is increased by two times, five times, ten times, fifty times, or one-hundred times compared to the binding affinity of the effector antigen binding domain of an antibody that lacks the target antigen binding domain.

14. The recombinant bispecific antibody of any one of claims 1-13, wherein a K_{d} for binding of the effector antigen binding domain to the antigen on the antigen presenting cell is less than 20 nM, less than 100 nM, or less than 500 nM.

15. The recombinant bispecific antibody of any one of claims 1-14, wherein the Fc comprising domain is linked to the target antigen binding domain and to the effector antigen binding domain.

16. The recombinant bispecific antibody of any one of claims 1-15, wherein the target antigen binding domain comprises an immunoglobulin heavy chain variable region or antigen binding fragment thereof and an immunoglobulin light chain variable region or antigen binding fragment thereof.

17. The recombinant bispecific antibody of any one of claims 1-16, wherein the target antigen binding domain comprises a single chain variable region fragment (scFv).

18. The recombinant bispecific antibody of any one of claims 1-17, wherein the tumor associated antigen is an antigen selected from the group consisting of CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PD-L, CD204, CD206, CD301, VTCN1, and VISTA.

19. The recombinant bispecific antibody of any one of claims 1-18, wherein the tumor associated antigen is Her2/neu or p185HER2.

20. The recombinant bispecific antibody of any one of claims 1-19, wherein the target antigen binding domain comprises the following CDRs:
a) HCDR1 comprising an amino acid sequence of SEQ ID NO: 13;
b) HCDR2 comprising an amino acid sequence of SEQ ID NO: 14;
c) HCDR3 comprising an amino acid sequence of SEQ ID NO: 15;
d) LCDR1 comprising an amino acid sequence of SEQ ID NO: 18;
e) LCDR2 comprising an amino acid sequence of SEQ ID NO: 19; and
f) LCDR3 comprising an amino acid sequence of SEQ ID NO: 20; and wherein the recombinant bispecific antibody specifically binds to Her2/neu or p185HER2.

21. The recombinant bispecific antibody of claim 20, wherein the target antigen binding domain comprises:
a) a VH sequence having at least 80% sequence identity to an amino acid sequence of SEQ ID NO: 12; and
b) a VL sequence having at least 80% sequence identity to an amino acid sequence of SEQ ID NO: 17.

22. The recombinant bispecific antibody of claim 20, wherein the target antigen binding domain comprises:
a) a heavy chain sequence having at least 80% sequence identity to an amino acid sequence of SEQ ID NO: 11; and
b) a light chain sequence having at least 80% sequence identity to an amino acid sequence of SEQ ID NO: 16.

23. The recombinant bispecific antibody of claim 20, wherein the target antigen binding domain comprises at least 80% sequence identity to the amino acid sequence between amino acid 20 and amino acid 110 of SEQ ID NO: 12 and at least 80% sequence identity to the amino acid sequence between amino acid 20 and amino acid 105 of SEQ ID NO: 17; and wherein the recombinant bispecific antibody specifically binds to Her2/neu or p185HER2.

24. The recombinant bispecific antibody of any one of claims 1-23, wherein the effector antigen binding domain comprises an immunoglobulin heavy chain variable region or antigen binding fragment thereof and an immunoglobulin light chain variable region or antigen binding fragment thereof.

25. The recombinant bispecific antibody of any one of claims 1-24, wherein the effector antigen binding domain comprises a single chain variable region fragment (scFv).

26. The recombinant bispecific antibody of claim 25, wherein the scFv comprises at least 80% sequence identity to an amino acid sequence of SEQ ID NO: 1312.

27. The recombinant bispecific antibody of any one of claims 1-26, wherein the antigen presenting cell is a dendritic cell.

28. The recombinant bispecific antibody on any one of claims 1-27, wherein the antigen on the antigen presenting cell is a costimulatory molecule.

29. The recombinant bispecific antibody of any one of claims 1-28, wherein the antigen on the antigen presenting cell is selected from the group consisting of CD40, OX40L, DEC-205, 4-1BBL, CD36, CD204, MARCO, DC-SIGN, CLEC9A, CLEC5A, Dectin 2, CLEC10A, CD206, CD64, CD32A, CD1A, HVEM, CD32B, PD-L1, or BDCA-2.

30. The recombinant bispecific antibody of any one of claims 1-29, wherein the effector antigen binding domain is a CD40 agonist.

31. The recombinant bispecific antibody of any one of claims 1-30, wherein the effector antigen binding domain comprises the following CDRs:
a) HCDR1 comprising an amino acid sequence of SEQ ID NO: 3;
b) HCDR2 comprising an amino acid sequence of SEQ ID NO: 4;
c) HCDR3 comprising an amino acid sequence of SEQ ID NO: 5;
d) LCDR1 comprising an amino acid sequence of SEQ ID NO: 8;
e) LCDR2 comprising an amino acid sequence of SEQ ID NO: 9; and
f) LCDR3 comprising an amino acid sequence of SEQ ID NO: 10.

32. The recombinant bispecific antibody of claim 31, wherein the effector antigen binding domain comprises:
a) a V_{H} sequence having at least 80% sequence identity to an amino acid sequence of SEQ ID NO: 2; and
b) a VL sequence having at least 80% sequence identity to an amino acid sequence of SEQ ID NO: 7.

33. The recombinant bispecific antibody of claim 31, wherein the effector antigen binding domain comprises:
a) a heavy chain sequence having at least 80% sequence identity to an amino acid sequence of SEQ ID NO: 1; and
b) a light chain having at least 80% sequence identity to an amino acid sequence of SEQ ID NO: 6.

34. The recombinant bispecific antibody of any one of claims 1-27, wherein the antigen on the antigen presenting cell is TREM2 or TNFR2.

35. The recombinant bispecific antibody of any one of claims 1-34, wherein the Fc comprising domain is linked C-terminal to the target antigen binding domain and N-terminal to the effector antigen binding domain.

36. The recombinant bispecific antibody of any one of claims 1-35, wherein the Fc comprising domain comprises one or more amino acid substitutions that reduce the affinity of the Fc comprising domain to an Fc receptor compared to the affinity of a reference Fc comprising domain to the Fc receptor in the absence of the one or more amino acid substitutions.

37. The recombinant bispecific antibody of claim 36, wherein reference Fc comprising domain is selected from the group consisting of an Fc comprising domain having the amino acid sequence of SEQ ID NO: 1314, SEQ ID NO: 1315, SEQ ID NO: 1316, and SEQ ID NO: 1317.

38. The recombinant bispecific antibody of claim 36, wherein reference Fc comprising domain comprises an amino acid sequence selected from the group consisting of SEQ ID NO: 1319, SEQ ID NO: 1320, SEQ ID NO: 1321, and SEQ ID NO: 1322.

39. The recombinant bispecific antibody of any one of claims 1-38, wherein the Fc comprising domain comprises a human IgG₁ Fc Region.

40. The recombinant bispecific antibody of claim 39, wherein the one or more amino acid substitutions comprise L234A, L235A, G237A, and K322A, according to the EU index of Kabat.

41. The recombinant bispecific antibody of claim 41, wherein the one or more amino acid substitutions comprise E233P, L234V, L235A, AG236, A327G, A330S, and P331S, according to the EU index of Kabat.

42. The recombinant bispecific antibody of any one of claims 1-36, wherein the Fc comprising domain comprises a human IgG₂ Fc Region.

43. The recombinant bispecific antibody of claim 42, wherein the one or more amino acid substitutions comprises K322A, according to the EU index of Kabat.

44. The recombinant bispecific antibody of any one of claims 1-36, wherein the Fc comprising domain comprises a human IgG₂ₐ Fc Region.

45. The recombinant bispecific antibody of claim 44, wherein the one or more amino acid substitutions comprises L235E, E318A, K320A, K322A, according to the EU index of Kabat.

46. The recombinant bispecific antibody of any of claims 1-36, wherein the Fc comprising domain is an Fc null.

47. The recombinant bispecific antibody of any one of claims 1-36 and 46, wherein the Fc comprising domain has the amino acid sequence of SEQ ID NO: 1313.

48. The recombinant bispecific antibody of any one of claims 1-36 and 46, wherein the Fc comprising domain comprises the amino acid sequence of SEQ ID NO: 1318.

49. The recombinant bispecific antibody of any one of claims 1-21, wherein the Fc comprising domain is linked C-terminal to the target antigen binding domain and has the amino acid sequence of SEQ ID NO: 1311.

50. The recombinant bispecific antibody of any one of claims 1 and 4-49, wherein the linker links the immune-stimulatory compound to the Fc comprising domain.

51. The recombinant bispecific antibody of any of claims 2, 3, and 5-50, further comprising an immune stimulatory compound and a linker, wherein the linker links the immune-stimulatory compound to the Fc comprising domain.

52. The recombinant bispecific antibody of any one of claims 1 and 4-52, wherein the immune-stimulatory compound is a damage-associated molecular pattern molecule or a pathogen-associated molecular pattern molecule.

53. The recombinant bispecific antibody of any one of claims 1 and 4-53, wherein the immune-stimulatory compound is a Toll-like receptor agonist, STING agonist, or RIG-I agonist.

54. The recombinant bispecific antibody of any one of claims 3-55, wherein the immune-stimulatory compound is a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663.

55. The recombinant bispecific antibody of any one of claims 1 and 4-53, wherein the immune-stimulatory compound is an inhibitor of TGFB, Beta-Catenin, PI3K-beta, STAT3, IL-10, IDO, or TDO.

56. The recombinant bispecific antibody of any one of claims 1 and 4-53, wherein the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843.

57. The recombinant bispecific antibody of any one of claims 1 and 4-56, wherein the immune-stimulatory compound does not reduce the affinity of the recombinant bispecific antibody for binding to the tumor associated antigen or to the antigen on the antigen presenting cell.

58. The recombinant bispecific antibody of any one of claims 1-57, further comprising a chemotherapeutic compound and a linker, wherein the linker links the chemotherapeutic compound to the Fc comprising domain.

59. The recombinant bispecific antibody of claim 58, wherein the chemotherapeutic compound comprises an alkylating agent, an anthracycline, a cytoskeletal disruptor, a histone deacetylase inhibitor, an inhibitor of, a kinase inhibitor, a nucleoside analog or precursor analog, a peptide antibiotic, a platinum-based compound, or a plant alkaloid.

60. A method of making a recombinant bispecific antibody comprising:
a) producing an antibody construct comprising:
i) a target antigen binding domain, wherein the target antigen binding domain specifically binds to a tumor associated antigen;
ii) an effector antigen binding domain, wherein the effector antigen binding domain specifically binds to an antigen on an antigen presenting cell and the antigen is a molecule on the antigen presenting cell, wherein the antigen is a molecule on the antigen presenting cell;
iii) an Fc comprising domain; and
b) linking an immune-stimulatory compound to the antibody construct, wherein the recombinant bispecific antibody induces greater immune cell activation when the recombinant bispecific antibody is bound to the tumor associated antigen and to the antigen on the antigen presenting cell as compared to when the recombinant bispecific antibody is bound to the antigen on the antigen presenting cell but not to the tumor associated antigen.

61. A pharmaceutical composition comprising the recombinant bispecific antibody of any of claims 1-59 and a pharmaceutically acceptable carrier.

62. A method of treating a subject in need thereof, comprising administering to the subject a therapeutic dose of the recombinant bispecific antibody of any of claims 1-59 or the pharmaceutical composition of claim 61.

63. The method of claim 62, wherein the subject has cancer.

64. The method of any one of claims 62-63, wherein the recombinant bispecific antibody or the pharmaceutical composition is administered intravenously, cutaneously, subcutaneously, or injected at a site of affliction.

65. The method of any one of claims 62-64, wherein the recombinant bispecific antibody induces greater immune activation against a cancer as measured by a decrease in cancer cell number or volume as compared to non-cancerous tissue.

66. The method of any one of claims 62-65, wherein when the recombinant bispecific antibody is administered intravenously to the subject at a minimum anticipated biological effect level of the recombinant bispecific antibody, a biological effect of the recombinant bispecific antibody is greater when the recombinant bispecific antibody is bound to the tumor associated antigen and to the antigen on the antigen presenting cell as compared to the biological effect of the recombinant bispecific antibody when it is not bound to the tumor associated antigen but is bound to the antigen on the antigen presenting cell; and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation.

67. The method of claim 66, wherein when the recombinant bispecific antibody is administered intravenously to the subject at the minimum anticipated biological effect level of the recombinant bispecific antibody, it induces a greater biological effect at the site of the cancer than at a non-cancerous site and wherein the biological effect is immune activation as measured by one or more of the group selected from secretion of one or more cytokines, secretion of one or more chemokines, expression level of one or more cell surface proteins associated with immune stimulation, antibody-dependent cell-mediated cytotoxicity, antibody dependent cellular phagocytosis, and antigen cross-presentation.

68. A conjugate comprising:
a) an antibody construct comprising:
i) first binding domain, wherein the first binding domain specifically binds to a tumor antigen;
ii) a second binding domain, wherein the second binding domain specifically binds to an antigen on an antigen presenting cell, wherein the antigen is a molecule on the antigen presenting cell; and
iii) an Fc domain;
b) an immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8;
wherein the first binding domain is attached to the Fc domain and the second binding domain is attached to the Fc domain or to a C-terminal end of a light chain of the first binding domain;
wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and
wherein immune cell activation caused by the conjugate when bound to the tumor antigen as measured by a cytokine release assay is greater than immune cell activation in the absence of binding to the tumor antigen.

69. A conjugate comprising:
a) an antibody construct comprising:
i) first binding domain, wherein the first binding domain specifically binds to a tumor antigen;
ii) a second binding domain, wherein the second binding domain specifically binds to an antigen on an antigen presenting cell, wherein the antigen is a molecule on the antigen presenting cell; and
iii) an Fc domain;
b) an immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8;
wherein the first binding domain is attached to the Fc domain and the second binding domain is attached to the Fc domain or to a C-terminal end of a light chain of the first binding domain;
wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and
wherein antigen presenting cells are conditionally activated when the conjugate is bound to the tumor antigen as measured by a cytokine release assay.

70. An antibody construct comprising:
a) a first binding domain, wherein the first binding domain specifically binds to a tumor antigen;
b) a second binding domain, wherein the second binding domain specifically binds to an antigen on an antigen presenting cell, wherein the antigen is a molecule on the antigen presenting cell; and
c) an Fc domain;
wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain.

71. An antibody construct for use in inducing immune cell activation comprising:
a) a first binding domain, wherein the first binding domain specifically binds to a tumor antigen;
b) a second binding domain, wherein the second binding domain specifically binds to an antigen on an antigen presenting cell, wherein the antigen is a molecule on the antigen presenting cell; and
c) an Fc domain;
wherein the first binding domain is attached to the Fc domain and the second binding domain is attached to the Fc domain or to a C-terminal end of a light chain of the first binding domain, and wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and
wherein immune cell activation caused by the antibody construct upon binding to tumor antigen as measured by a cytokine release assay is greater than immune cell activation caused by the antibody construct in the absence of binding to tumor antigen.

72. A conjugate for use in inducing immune cell activation comprising:
a) an antibody construct comprising:
i) first binding domain, wherein the first binding domain specifically binds to a tumor antigen;
ii) a second binding domain, wherein the second binding domain specifically binds to an antigen on an antigen presenting cell, wherein the antigen is a molecule on the antigen presenting cell; and
iii) an Fc domain;
b) an immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8;
wherein the first binding domain is attached to the Fc domain and the second binding domain is attached to the Fc domain or to a C-terminal end of a light chain of the first binding domain;
wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and
wherein immune cell activation caused by the conjugate when bound to the tumor antigen as measured by a cytokine release assay is greater than immune cell activation in the absence of binding to the tumor antigen.

73. A conjugate for use in conditionally activating an antigen presenting cell comprising:
a) an antibody construct comprising:
i) first binding domain, wherein the first binding domain specifically binds to a tumor antigen;
ii) a second binding domain, wherein the second binding domain specifically binds to an antigen on the antigen presenting cell, and
iii) an Fc domain;
b) an immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8;
wherein the first binding domain is attached to the Fc domain and the second binding domain is attached to the Fc domain or to a C-terminal end of a light chain of the first binding domain;
wherein a K_{d} for binding of the Fc domain to an Fc receptor in a presence of the first binding domain and the second binding domain is no greater than about 100 times a K_{d} for binding of the Fc domain to the Fc receptor in an absence of the second binding domain; and
wherein antigen presenting cells are conditionally activated when the conjugate is bound to the tumor antigen as measured cytokine release assay.

74. The conjugate of any one of claims 68-69 or 72-73, wherein a K_{d} for binding of the first binding domain to the tumor antigen in the presence of the immune-stimulatory compound is no greater than about two times, five times, ten times, or fifty times a K_{d} for binding of the first binding domain to the tumor antigen in an absence of the immune-stimulatory compound.

75. The conjugate of any one of claims 68-69 or 72-74, wherein a Kd for binding of the second binding domain to the antigen on the antigen presenting cell in the presence of the immune-stimulatory compound is no greater than about two times, five times, ten times, or fifty times a Kd for binding of the second binding domain to the antigen on the antigen presenting cell in an absence of the immune-stimulatory compound.

76. The antibody construct or conjugate of any one of claims 68-75, wherein a Kd for binding of the first binding domain to the tumor antigen is no greater than about 100 nM.

77. The antibody construct or conjugate of any one of claims 68-76, wherein a Kd for binding of the second binding domain to the antigen on an antigen presenting cell is no greater than about 100 nM.

78. The antibody construct or conjugate of any one of claims 68-77, wherein an amino acid sequence of the tumor antigen has at least 80% sequence identity with the amino acid sequence of a tumor antigen selected from the group consisting of HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, Her-2/neu, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, C1orf186, Fos-related antigen 1, VEGFR1, endoglin, PD-L, VTCN1 (B7-H4), VISTA, or a fragment thereof, and a fragment thereof.

79. The antibody construct or conjugate of any one of claims 68-79, wherein an amino acid sequence of the tumor antigen has at least 80% sequence identity with the amino acid sequence of a tumor antigen selected from TABLE 1.

80. The antibody construct or conjugate of any one of claims 68-77, wherein an amino acid sequence of the tumor antigen has at least 80% sequence identity with the amino acid sequence of a tumor antigen selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15, but not HER2 when the second binding domain specifically binds to CD40.

81. The antibody construct or conjugate of any one of claims 68-81, wherein an amino acid sequence of the antigen on the antigen presenting cell has at least 80% sequence identity with the amino acid sequence of an antigen selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, CD32B, and CD47, but not CD40 when the first binding domain specifically binds to HER2.

82. The antibody construct or conjugate of any one of claims 68-81, wherein an amino acid sequence of the antigen on the antigen presenting cell has at least 80% sequence identity with the amino acid sequence of an antigen selected from TABLE 2.

83. The antibody construct or conjugate of any one of claims 68-82, wherein the second binding domain is a CD40 agonist.

84. The antibody construct or conjugate of any one of claims 68-83, wherein the first binding domain comprises a single chain variable fragment (scFv).

85. The antibody construct or conjugate of any one of claims 68-84, wherein the second binding domain is a single chain variable fragment (scFv).

86. The antibody construct or conjugate of any one of claims 68-85, wherein the second binding domain comprises a single chain variable fragment from an anti-CD40 antibody, an anti-DEC-205 antibody, an anti-CD36 mannose scavenger receptor 1 antibody, an anti-DC-SIGN antibody, an anti-CLEC9A antibody, an anti-CLEC12A antibody, an anti-BDCA-2 antibody, an anti-OX40L antibody, an anti-41BBL antibody, an anti-CD204 antibody, an anti-MARCO antibody, an anti-CLEC5A antibody, an anti-Dectin 1 antibody, an anti-Dectin 2 antibody, an anti-CLEC10A antibody, an anti-CD206 antibody, an anti-CD64 antibody, an anti-CD32A antibody, an anti-CD16A antibody, an anti-HVEM antibody, an anti-PD-L1, or an anti-CD32B antibody.

87. The antibody construct or conjugate of any one of claims 68-86, wherein the second binding domain is attached to the Fc domain or the light chain of the first binding domain:
a) as an Fc domain-second binding domain fusion peptide;
b) as a light chain-second binding domain fusion peptide; or
c) by a conjugation via a first linker.

88. The antibody construct or conjugate of any one of claims 68-87, wherein the Fc domain is attached to the first binding domain:
a) as an Fc domain-first binding domain fusion peptide; or
b) by conjugation via a second linker.

89. The antibody construct or conjugate of any one of claims 68-88, wherein the Fc domain is attached to both the first binding domain and to the second binding domain as a first binding domain-Fc domain-second binding domain fusion peptide.

90. The antibody construct or conjugate of any one of claims 68-89, wherein the first binding domain is attached to both the Fc domain and the second binding domain as a first binding domain-second binding domain-Fc domain fusion peptide.

91. The antibody construct or conjugate of any one of claims 68-90, wherein the first binding domain and the Fc domain comprise an antibody and the second binding domain comprises a single chain variable fragment (scFv).

92. The antibody construct or conjugate of any one of claims 68-91, wherein the first binding domain has a set of variable region CDR sequences that comprises a set of variable region CDR sequences set forth in TABLE 3 or TABLE 4.

93. The antibody construct or conjugate of any one of claims 68-92, wherein the second binding domain comprises a variable domain comprising a set of CDR sequences set forth in TABLE 11 or TABLE 12.

94. The antibody construct or conjugate of any one of claims 68-93, wherein the first binding domain comprises a variable region comprising VH and VL sequences at least 80% sequence identity to a pair of VH and VL sequences set forth in TABLE 5 or TABLE 6.

95. The antibody construct or conjugate of any one of claims 68-94, wherein the second binding domain comprises a variable region having VH and VL sequences having at least 80% sequence identity to a VH or VL sequence set forth in TABLE 13 or TABLE 14.

96. The antibody construct or conjugate of any one of claims 68-95, wherein the first binding domain comprises an amino acid sequence having at least 80% sequence identity to any sequence in TABLE 7 or TABLE 8.

97. The antibody construct or conjugate of any one of claims 68-96, wherein the second binding domain comprises an amino acid sequence having at least 80% sequence identity to any sequence in TABLE 15 or TABLE 16.

98. The second binding domain-Fc domain-first binding domain fusion peptide of claim 89 comprising an amino acid sequence having at least 80% sequence identity to a sequence in TABLE 9, TABLE 10, or TABLE 17.

99. The second binding domain-first binding domain-Fc domain fusion peptide of claim 90 comprising an amino acid sequence having at least 80% sequence identity to a sequence in TABLE 18 or TABLE 19.

100. A conjugate comprising:
a) an immune-stimulatory compound;
b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, 41BBL, MARCO, CLEC5A, Dectin 1, Dectin 2, CD64, CD32A, CD16A, HVEM, and CD32B, and a fragment thereof; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

101. A conjugate comprising:
a) an immune-stimulatory compound;
b) an antibody construct comprising a first binding domain and an Fc domain, wherein:
i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, OX40L, 41BBL, MARCO, CLEC5A, Dectin 1, Dectin 2, CD64, CD32A, CD16A, HVEM, CD32B, and CD47, and a fragment thereof,
ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and
iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

102. A conjugate comprising:
a) an immune-stimulatory compound;
b) an antibody construct comprising a first binding domain and an Fc domain, wherein:
i) the first binding domain comprises a variable region comprising a set of CDR sequences that comprises at least 80% sequence identity to a set of variable region CDR sequences set forth in TABLE 3 or TABLE 11;
ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and
iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8.

103. A conjugate for use in activating an immune cell comprising:
a) an immune-stimulatory compound;
b) an antibody construct comprising a first binding domain and an Fc domain, wherein the first binding domain specifically binds to an antigen expressed on a cell, wherein the amino acid sequence of the antigen has at least 80% homology to the amino acid sequence of an antigen selected from a group consisting of Endoglin, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, 41BBL, MARCO, CLEC5A, Dectin 1, Dectin 2, CD64, CD32A, CD16A, HVEM, and CD32B, and a fragment thereof; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and
wherein immune cell activation caused by the conjugate when bound to the tumor antigen as measured by a cytokine release assay is greater than immune cell activation is greater than immune cell activation in the absence of binding to the tumor antigen.

104. A conjugate for use in activating an immune cell comprising:
a) an immune-stimulatory compound;
b) an antibody construct comprising a first binding domain and an Fc domain, wherein:
i) the first binding domain specifically binds to an antigen, wherein the amino acid sequence of the antigen has at least 80% homology to the amino acid sequence of an antigen selected from a group consisting of endoglin, PD-L1, CD204, CD206, CD301, VTCN1, VISTA, GLP-3, CLDN6, CLDN16, UPK1B, STRA6, TMPRSS3, TMPRSS4, TMEM238, C1orf186, LRRC15, DEC-205, CD36 mannose scavenger receptor 1, CLEC9A, DC-SIGN, CLEC12A, BDCA-2, OX40L, 41BBL, MARCO, CLEC5A, Dectin 1, Dectin 2, CD64, CD32A, CD16A, HVEM, CD32B, and CD47, and a fragment thereof,
ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and
iii) a K_{d} for binding of the Fc domain to an Fc receptor in the presence of the immune-stimulatory compound is no greater than about 100 times a K_{d} for binding of the Fc domain to the Fc receptor in the absence of the immune-stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and
wherein immune cell activation caused by the conjugate when bound to the tumor antigen as measured by a cytokine release assay is greater than immune cell activation is greater than immune cell activation in the absence of binding to the tumor antigen.

105. A conjugate for use in activating an immune cell comprising:
a) an immune-stimulatory compound;
b) an antibody construct comprising a first binding domain and an Fc domain, wherein the first binding domain comprises a variable region comprising a set of CDR sequences that comprises at least 80% sequence identity to a set of variable region CDR sequences set forth in TABLE 3 or TABLE 11;
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and
wherein immune cell activation caused by the conjugate when bound to the tumor antigen as measured by a cytokine release assay is greater than immune cell activation is greater than immune cell activation in the absence of binding to the tumor antigen.

106. A conjugate for use in activating an immune cell comprising:
a) an immune-stimulatory compound;
b) an antibody construct comprising a first binding domain and an Fc domain, wherein:
i) the first binding domain comprises a variable region comprising a set of CDR sequences that comprises at least 80% sequence identity to a set of variable region CDR sequences set forth in TABLE 3 or TABLE 11;
ii) a K_{d} for binding of the first binding domain to the antigen in a presence of the immune-stimulatory compound is less than about 100 nM and no greater than about 100 times a K_{d} for binding of the first binding domain to the antigen in the absence of the immune-stimulatory compound, and
iii) a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and
c) a linker attaching the antibody construct to the immune-stimulatory compound, wherein the linker is covalently bound to the antibody construct and the linker is covalently bound to the immune-stimulatory compound, and wherein a molar ratio of immune-stimulatory compound to antibody construct is less than 8; and
wherein immune cell activation caused by the conjugate when bound to the tumor antigen as measured by a cytokine release assay is greater than immune cell activation is greater than immune cell activation in the absence of binding to the tumor antigen.

107. The conjugate of any one of claims 100-106, wherein the first binding domain comprises a variable region comprising V_{H} and V_{L} sequences at least 80% sequence identity to a pair of V_{H} and V_{L} sequences set forth in TABLE 5 or TABLE 13.

108. The conjugate of any one of claims 100-107, wherein the first binding domain comprises an amino acid sequence having at least 80% sequence identity to any sequence in TABLE 7 or TABLE 15.

109. The conjugate of any one of claims 68-69 or 72-108, wherein a Kd for binding of the Fc domain to the Fc receptor in the presence of the immune-stimulatory compound is no greater than about two times, five times, ten times, or fifty times a Kd for binding of the Fc domain to the Fc receptor in an absence of the immune-stimulatory compound.

110. The conjugate of any one of claims 68-69 or 72-109, wherein the immune-stimulatory compound is a damage-associated molecular pattern molecule or pathogen-associated molecular pattern molecule.

111. The conjugate of any one of claims 68-69 or 72-110, wherein the immune-stimulatory compound is a toll-like receptor agonist, STING agonist, or RIG-I agonist.

112. The conjugate of any one of claims 68-69 or 72-111, wherein the immune-stimulatory compound is a CpG oligonucleotide, Poly G10, Poly G3, Poly I:C, Lipopolysaccharide, zymosan, flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663.

113. The conjugate of any one of claims 68-69 or 72-109, wherein the immune-stimulatory compound is an inhibitor of TGFB, Beta-Catenin, TNIK, Tankyrase, PI3K-beta, STAT3, IL-10, IDO, or TDO.

114. The conjugate of any one of claims 68-69 or 72-109, wherein the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843.

115. The antibody construct or conjugate of any one of claims 68-69 or 72-114, wherein the Fc domain is an Fc domain variant comprising at least one amino acid residue change as compared to a wild type sequence of the Fc domain.

116. The antibody construct or conjugate of claim 115, wherein the Fc domain variant binds to an Fc receptor with altered affinity as compared to the wild type Fc domain.

117. The antibody construct or conjugate of any one of claims 115-116, wherein the at least one amino acid residue change is selected from a group consisting of:
a) F243L, R292P, Y300L, L235V, and P396L, wherein numbering of amino acid residues in the Fc domain is according to the EU index;
b) S239D and I332E, wherein numbering of amino acid residues in the Fc domain is according to the EU index; and
c) S298A, E333A, and K334A, wherein numbering of amino acid residues in the Fc domain is according to the EU index.

118. The antibody construct or conjugate of any one of claims 68-117, wherein the antibody construct or conjugate induces secretion of cytokines by an immune cell as measured by a cytokine release assay.

119. The antibody construct or conjugate of claim 118, wherein the cytokine is IFN-γ, IL-8, IL-12, IL-2, or a combination thereof.

120. The antibody construct or conjugate of any one of claims 68-119, wherein the antibody construct or conjugate induces antigen presentation on a dendritic cell, B cell, macrophage, or a combination thereof.

121. A method of making a conjugate comprising linking an antibody construct of any one of claims 70-71, 76-99, or 116-120 to an immune stimulatory compound by a linker.

122. A pharmaceutical composition comprising the conjugate or antibody construct of any of claims 68-120 and a pharmaceutically acceptable carrier.

123. A method of treatment for a subject in need thereof, comprising administering a therapeutic dose of the antibody construct or conjugate of any one of claims 68-120 or the pharmaceutical composition of claim 122.

124. The method of claim 123, wherein the subject has cancer.

125. The method of any one of claims 123-124, wherein antibody construct or conjugate is administered intravenously, cutaneously, subcutaneously, or injected at a site of affliction.

126. The method of any one of claims 123-125, wherein after administration of antibody construct or conjugate to the subject, immune cell activation is increased in the subject as measured by a secretion of one or more cytokines as measured by a cytokine release assay, a secretion of one or more chemokines as measured by an ELISA immunoassay, an expression level of one or more cell surface proteins associated with immune stimulation as measured by an ELISA immunoassay, an activity of one or more immune cell functions, or combination thereof, as compared to before administration of the antibody construct or conjugate to the subject.

127. The method of claim 126, wherein the activity of one or more immune cell functions comprises antibody-dependent cell-mediated cytotoxicity as measured by an ADCC assay, antibody dependent cellular phagocytosis as measured by an ADCP assay, or antigen cross-presentation as measured by a cross-presentation assay.

128. The method of any one of claims 123-127, wherein after administration of the antibody construct or conjugate to the subject, tumor cell intracellular signaling is altered in the subject as compared to tumor cell intracellular signaling before administration of the antibody construct or conjugate as measured by an intracellular signaling assay.

129. The method of claim 128, wherein the altered tumor cell intracellular signaling increases tumor immunogenicity as measured by an immunogencity assay.

130. A kit comprising a pharmaceutically acceptable dosage unit of a pharmaceutically effective amount of the conjugate or antibody construct according to any of claims 1-120 or the pharmaceutical composition of claim 122.
